(19) United States
(12) Patent Application Publication
(10) Pub. No.: US 2026/0043030 A1
(43) Pub. Date: Feb. 12, 2026

(54) Oligonucleotide for reducing the expression of leucine-rich repeat kinase 2 (LRRK2) and its use for preventing and/or treating human diseases

(71) Applicants: Secarna Pharmaceuticals GmbH & Co. KG, Marburg (DE); SciNeuro Therapeutics Inc, Rockville, MD (US)

(72) Inventors: Marta Lucia DE LOS REYES, München (DE); Richard KLAR, München (DE); Sven MICHEL, Bernried (DE); Frank JASCHINSKI, Puchheim (DE); Irene RIERA, Munich (DE); Min LI, Vedra Beach, FL (US); Andreas ZEMBRZYCKI, San Diego, CA (US); Dai-Shi SU, Dresher, PA (US)

(21) Appl. No.: 18/851,888

(22) PCT Filed: Apr. 6, 2023
(86) PCT No.: PCT/EP2023/059279

§ 371 Date: Sep. 27, 2024

(30) Foreign Application Priority Data
Apr. 8, 2022 (EP) 22167394.0

(51) Int. Cl. C12N 15/113

(52) U.S. Cl. CPC C12N 15/1137; C12N 2310/14; C12N 2310/3231

(57) ABSTRACT

The present invention refers to an oligonucleotide comprising 10 to 25 nucleotides, at least one nucleotide having a modification, wherein the oligonucleotide hybridizes with a transcript or an mRNA of leucine-rich repeat kinase 2 (LRRK2) of SEQ ID NO. 1 and/or with pre-mRNA of LRRK2 of SEQ ID NO. 2 and/or a fragment thereof. The invention further refers to a pharmaceutical composition comprising such oligonucleotide, and the use of the oligonucleotide and pharmaceutical composition, respectively, for use in method of preventing and/or treating a human disease.

The present invention refers to oligonucleotides, in particular antisense oligonucleotides, comprising one or more modified nucleotides and hybridizing with the LRRK2 mRNA and/or pre-mRNA. The oligonucleotides inhibit the expression of LRRK2, LRRK2 mRNA and/or LRRK2 pre-mRNA and are used for preventing and/or treating a human disease such as Parkinson's disease (PD) and other neurodegenerative diseases.

### TECHNICAL BACKGROUND

A neurodegenerative disease is caused by the progressive loss of structure or function of neurons, in the process known as neurodegeneration. Such neuronal damage may ultimately involve cell death. Neurodegenerative diseases include amyotrophic lateral sclerosis, multiple sclerosis, Parkinson's disease, Alzheimer's disease, Huntington's disease, multiple system atrophy, and prion diseases. Neurodegeneration can be found in the brain at many different levels of neuronal circuitry.

Parkinson's disease (PD) is the second most common neurodegenerative disease and affects approximately 1 in 100 people over the age of 60. A disease-modifying or neuroprotective therapy is highly desired, as the current standard of care is limited to symptomatic treatment (Lees, Hardy, & Revesz, 2009). Pathological hallmarks of PD include accumulation of α-synuclein (α-syn) into Lewy bodies (LBs) and Lewy neurites (LNs), and progressive loss of dopaminergic (DA) neurons in the substantia nigra pars compacta (SNpc) (Trojanowski & Lee, 1998).

Currently a variety of medications provide some relief from the symptoms of the neurodegenerative diseases such as Parkinson's disease. However, there is a lack of effective/acceptable treatment for such disease.

Understanding of the genetics of PD has identified a number of genetic alleles causing or associated with disease on-set and progression (Chang et al., 2017; Gasser, Hardy, & Mizuno, 2011; Hardy, 2010; Lunati, Lesage, & Brice, 2018). The human genetic evidence demonstrates a direct involvement of LRRK2 function in risk to develop PD, the underlying disease pathology and progression of PD and thus validate LRRK2 as an attractive therapeutic target. LRRK2 is a 2527 amino acid large protein that is widely expressed throughout the body with its highest level being detected in lung and kidney tissues. The LRKK2 protein consists of multiple functional domains, including a Ras of complex proteins (ROC)-type GTP-binding domain, a carboxyl terminal of ROC (COR) domain, and a serine/threonine protein kinase domain (Cookson, 2017). Collectively, LRRK2 appears to regulate aspects of intracellular vesicle trafficking, maintenance of cellular organelles including the Golgi, endosomes and lysosomes.

Altered LRRK2 functions due to genetic, epigenetic or environmental factors, and similarly mutations in or around the LRRK2 gene are directly involved in development and progression of diseases, including synucleopathies such as PD. Therefore, suppression of its expression could provide an effective treatment opportunity for these diseases. Suppression of LRRK2 expression can be achieved for example by targeted degradation of the mRNA coding for LRRK2 or by blocking of its translation.

Two competing technologies are predominantly described for specific suppression of mRNA expression: siRNA and antisense oligonucleotides (ASOs). siRNAs target the mRNA in the cytoplasm. In contrast, RNase H dependent ASOs target the mRNA and/or pre-mRNA in the nucleus. Thereby also intronic regions can be targeted by ASOs that are not accessible to siRNA resulting in a significantly larger target space for ASOs.

Moreover, due to its double stranded nature, siRNA does not cross the cell membrane by itself and delivery systems are required for its activity in vitro and in vivo. While delivery systems for siRNA exist that efficiently deliver siRNA to liver cells in vivo, there is currently no system that can deliver siRNA in vivo to extra-hepatic tissues with sufficient efficacy. In contrast, modified ASOs can enter many cell types in vitro without transfection or delivery methods in sufficient amounts, resulting in potent and sequence specific target knockdown. In vivo, naked unconjugated ASOs achieve target knockdown in several different relevant tissues after systemic administration. After direct administration, ASOs achieve a potent and long-lasting target knockdown in the central nervous system (CNS) indicating the strong potential of ASOs for treatment of CNS diseases such as neurodegenerative diseases (e.g., Geary et al., Adv Drug Deliv Rev. 2015 Jun. 29; 87:46-51).

The present invention refers to oligonucleotides such as antisense oligonucleotides (ASOs) resulting in significant reduction of LRRK2 mRNA and protein expressions in CNS as a therapeutic treatment option for PD.

The mode of action of an oligonucleotide differs from the mode of action of an antibody or small molecule, and oligonucleotides are highly advantageous regarding, for example,
(i) the penetration of tissue such as brain tissue which is better than penetration of antibodies due to the substantially smaller size (e.g., Pizzo et al., J Physiol. 2018 Feb. 1; 596(3):445-475; Noguchi et al., MAbs. 2017 October; 9(7): 1210-1215),
(ii) prolonged retention and effect duration in the brain after direct administration,
(iii) the blocking of multiple functions and activities, respectively, of a target,
(iv) the combination of oligonucleotides with each other or an antibody or a small molecule, and
(v) the inhibition of intracellular effects which are not accessible for an antibody or not inhibitable via a small molecule.

Thus, a need exists for new and improved therapies for the treatment of disease (e.g., PD) that are capable of reducing the amount of LRRK2 mRNA and protein, with minimal toxic side effects.

### SUMMARY OF THE INVENTION

The present invention refers to an oligonucleotide comprising 10 to 25 nucleotides, at least one nucleotide having a modification selected from the group consisting of a bridged nucleic acid such as LNA, ENA, cET, a 2′Fluoro modified nucleotide, a 2′ O-Methyl modified nucleotide, a 2′ O-Methoxy modified nucleotide, a FANA and a combination thereof, wherein said modification is located in a sequence of 5 nucleotides of the 5′-end and/or 3′-end of the oligonucleotide and wherein the oligonucleotide hybridizes with a transcript or an mRNA of leucine-rich repeat kinase 2 (LRRK2) of SEQ ID NO. 1 and/or with pre-mRNA of LRRK2 of SEQ ID NO. 2 and/or a fragment thereof. The fragment results from a biological process including but not limited to alternative splicing. The oligonucleotide hybridizes, for example, outside a hybridizing active region or within a hybridizing active region of position 102001 to 102500, position 27001 to 27500, position 80501 to 81000, position 24501 to 25000, position 114001 to 114500, position 92001 to 92500, position 99501 to 100000, position 67001 to 67500, position 15001 to 15500, position 24001 to 24500, position 26501 to 27000, position 79501 to 80000, position 87001 to 87500, position 120501 to 121000, position 142001 to 142500, position 135001 to 135500, position 71001 to 71500, position 140501 to 141000 or a combination thereof. The oligonucleotide comprises or consists of, for example, SEQ ID NO. 210, SEQ ID NO. 69, SEQ ID NO. 164, SEQ ID NO. 254, SEQ ID NO. 222, SEQ ID NO. 186, SEQ ID NO. 204, SEQ ID NO. 142, SEQ ID NO. 209, SEQ ID NO. 255, SEQ ID NO. 45, SEQ ID NO. 46, SEQ ID NO. 60, SEQ ID NO. 66, SEQ ID NO. 162, SEQ ID NO. 171, SEQ ID NO. 224, SEQ ID NO. 241, SEQ ID NO. 250, SEQ ID NO. 149, SEQ ID NO. 256, SEQ ID NO. 239, or a combination thereof. The sequence comprises at least one modification.

For example the oligonucleotide is selected from the group consisting of

(A57208Hi; SEQ ID NO. 210)
+T*+A*+C*A*A*T*A*T*T*T*A*T*G*G*T*T*+C*+T*+C,
(A57067Hi; SEQ ID NO. 69)
+A*+A*+T*A*C*T*A*C*T*A*T*T*T*G*T*T*+T*+C*+C,
(A57162Hi; SEQ ID NO. 164)
+G*+T*+C*A*T*A*A*G*T*T*G*T*C*A*G*+T*+A*+T,
(A57252H; SEQ ID NO. 254)
+G*+A*+C*T*T*C*G*T*T*T*A*A*T*A*C*+C*+A*+G,
(A57220Hi; SEQ ID NO. 222)
+C*+T*+T*T*G*C*T*T*A*T*T*C*G*T*G*+A*+C*+T,
(A57184Hi; SEQ ID NO. 186)
+T*+T*+T*A*T*T*G*C*A*G*T*C*A*T*A*G*+T*+G*+A,
(A57202Hi; SEQ ID NO. 204)
+C*+T*+T*T*G*T*A*A*A*T*G*A*C*G*T*T*+T*+C*+C,
(A57140Hi, SEQ ID NO. 142)
+T*+G*+A*A*A*T*G*T*C*T*A*T*T*A*G*C*+A*+C*+A,
(A57207Hi; SEQ ID NO. 209)
+A*+C*+A*A*T*A*T*T*T*A*T*G*G*T*T*C*+T*+C*+A,
(A57253H; SEQ ID NO. 255)
+G*+G*+A*C*T*T*C*G*T*T*T*A*A*T*A*C*+C*+A*+G,
(A57043Hi; SEQ ID NO. 45)
+C*+G*+G*C*T*A*A*A*T*G*T*T*T*C*A*G*+T*+T*+C,
(A57044Hi; SEQ ID NO. 46)
+T*+C*+G*G*C*T*A*A*A*T*G*T*T*T*C*+A*+G*+T,
(A57058Hi; SEQ ID NO. 60)
+G*+T*+G*T*A*A*C*T*A*A*G*T*G*A*T*+C*+T*+C,
(A57064Hi; SEQ ID NO. 66)
+G*+A*+T*A*T*T*G*A*C*A*A*C*C*T*G*+C*+T*+G,
(A57160Hi; SEQ ID NO. 162)
+T*+G*+T*G*T*T*A*C*C*A*T*A*T*C*C*A*+T*+T*+T,
(A57169Hi; SEQ ID NO. 171)
+C*+T*+G*T*G*A*C*T*T*T*G*C*A*T*C*A*+A*+C*+T,
(A57222Hi; SEQ ID NO. 224)
+C*+A*+A*T*G*T*A*T*A*C*C*A*T*G*C*+A*+G*+T,
(A57239Hi; SEQ ID NO. 241)
+T*+T*+C*C*T*G*A*T*T*A*T*A*C*C*A*T*+A*+T*+G,
(A57248Hi; SEQ ID NO. 250)
+A*+T*+G*C*T*C*T*T*A*T*C*C*A*C*A*A*+T*+C*+C,
(A57147Hi; SEQ ID NO. 149)
+T*+T*+T*G*A*T*C*T*G*T*A*G*T*A*C*A*+T*+A*+C,
(A57254H; SEQ ID NO. 256)
+A*+T*+G*G*A*C*T*T*C*G*T*T*T*A*A*T*+A*+C*+C,
(A57237Hi; SEQ ID NO. 239)
+G*+G*+A*C*A*T*G*T*A*T*C*T*T*A*A*T*+A*+G*+T,

and a combination thereof, wherein + indicates an LNA nucleotide and * indicates a phosphorothioate (PTO) linkage between the nucleotides. Alternatively, the oligonucleotide has 80 to 99%, 85 to 98%, 90 to 95 or 93% sequence identity to any one of the oligonucleotides of SEQ ID NO. 3 to SEQ ID NO. 5301 of the present invention.

Furthermore, the oligonucleotide comprises or consists of, for example, at least 7, at least 8, at least 9, at least 10, at least 11, at least 12, at least 13, at least 14, at least 15, at least 16, at least 17, at least 18, at least 19, at least 20, at least 21 or at least 22 nucleotides of any one of the oligonucleotides of SEQ ID NO. 3-SEQ ID NO. 5301 of the present invention. The nucleotides are for example contiguous nucleotides.

The oligonucleotide of the present invention inhibits, for example, the expression of LRRK2, a LRRK2 mRNA, a LRRK2 pre-mRNA or a combination thereof at a nanomolar or micromolar concentration.

The present invention is further directed to a pharmaceutical composition comprising or consisting of an oligonucleotide of the present invention and a pharmaceutically acceptable carrier, excipient, dilutant, stimulant such as an adjuvant, or a combination thereof. The pharmaceutical composition can comprise an additional therapeutically active agent. The oligonucleotide and the pharmaceutical composition, respectively, of the present invention is, for example, for use in a method of preventing and/or treating a human disease such as a neurodegenerative disease. The neurodegenerative disease is, for example, selected from the group consisting of Parkinson's disease (PD), Alzheimer's disease, Huntington's disease, amyotrophic lateral sclerosis, multiple sclerosis, multiple system atrophy, dementia with Lewy bodies (DLB), pure autonomic failure (PAF), frontotemporal dementia and prion disease. The pharmaceutically acceptable carrier, excipient, adjuvant, or a combination thereof, or the therapeutically active agent is for example administrated separately before or after delivery of the oligonucleotide, or is for example administered simultaneously via different delivery methods. The delivery methods are for example orally, sublingually, nasally, subcutaneously, intravenously, intraperitoneally, intramuscularly, intratumorally, intrathecally, intraventricularly, transdermal and/or rectally.

The oligonucleotide and pharmaceutical composition, respectively, of the present invention is, for example, for local or systemic administration.

The present invention further relates to a method of preventing and/or treating a human disease comprising administering a therapeutically effective amount of an oligonucleotide of the present invention or a pharmaceutical composition comprising such oligonucleotide. The human disease, such as a neurological disease, can be selected from the group consisting of Parkinson's disease (PD), Alzheimer's disease, Huntington's disease, amyotrophic lateral sclerosis, multiple sclerosis, multiple system atrophy, dementia with Lewy bodies (DLB), pure autonomic failure (PAF), frontotemporal dementia and prion disease. The oligonucleotide or pharmaceutical composition can be administered locally or systemically.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF FIGURES

FIG. 1 shows RefSeq ID NM_198578 of LRRK2 mRNA (SEQ ID NO. 1).

FIG. 2 depicts GRCh38:12:40224997:40369285, in particular LRRK2_GRCh38:12:40224997:40369285:1_seqGenomicENST00000298910.12 (LRRK2 pre-mRNA of SEQ ID NO. 2).

FIG. 3A to 3D depict the screening of human LRRK2-specific ASOs in A549 cells (human lung carcinoma).

FIGS. 4A to 4D show the screening of human LRRK2-specific ASOs in MDA-MB-231 cells (human breast adenocarcinoma).

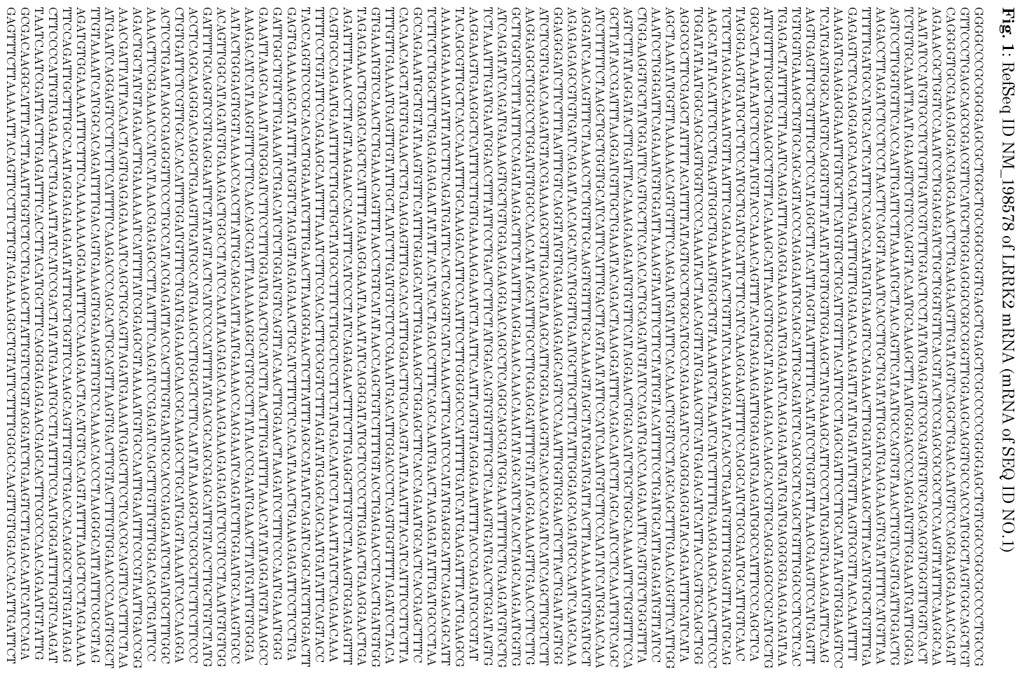

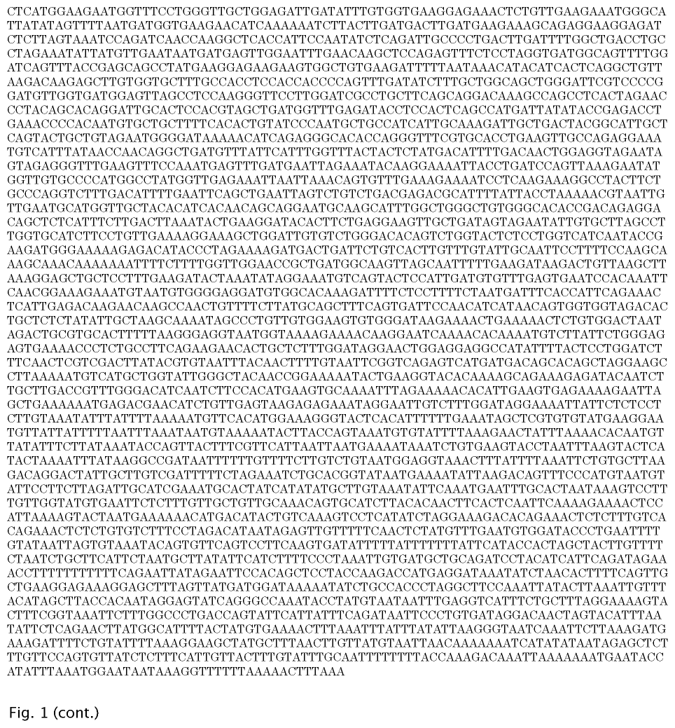

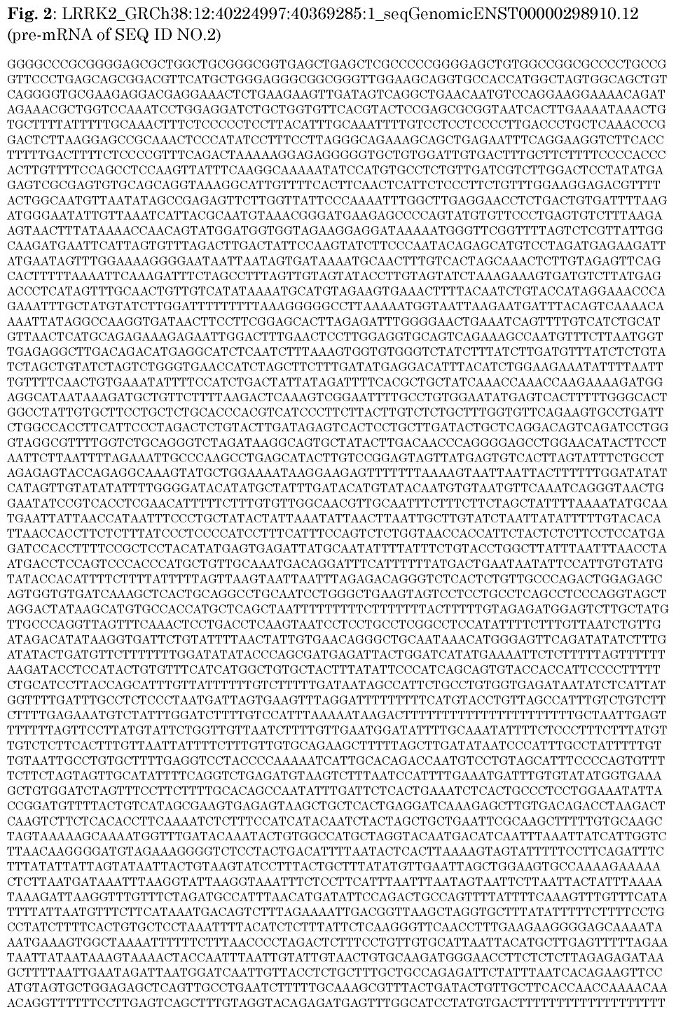

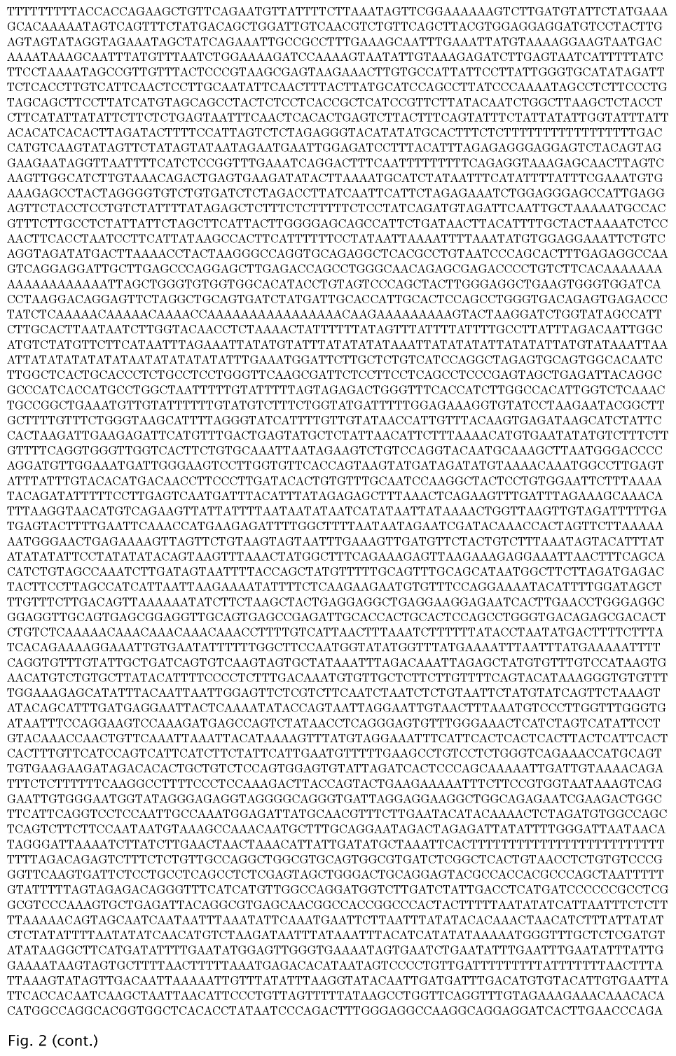

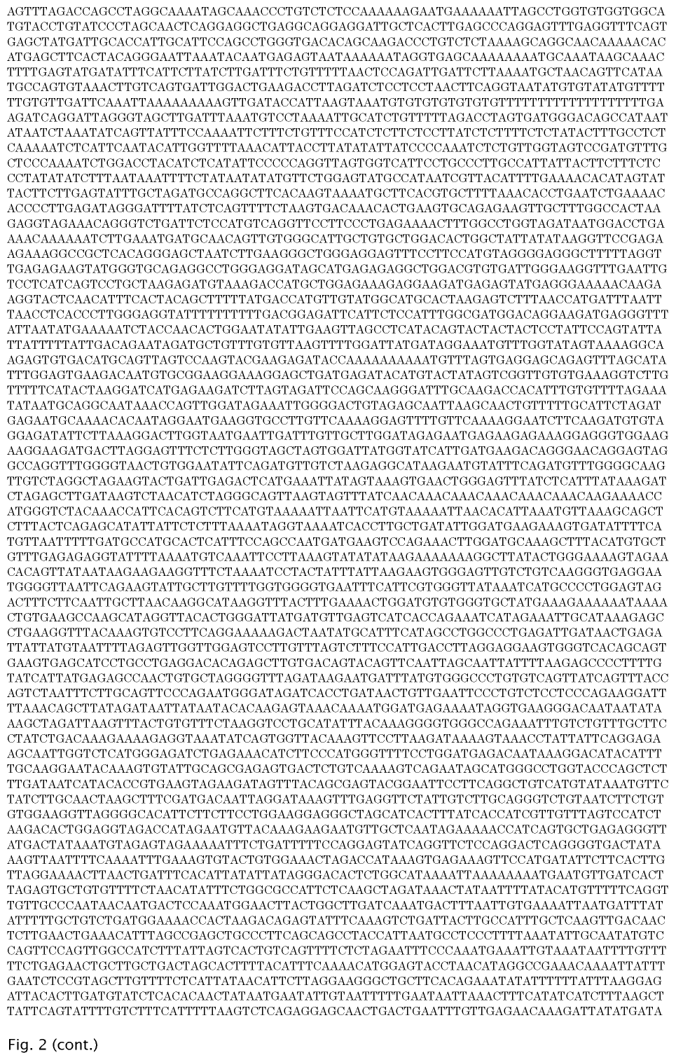

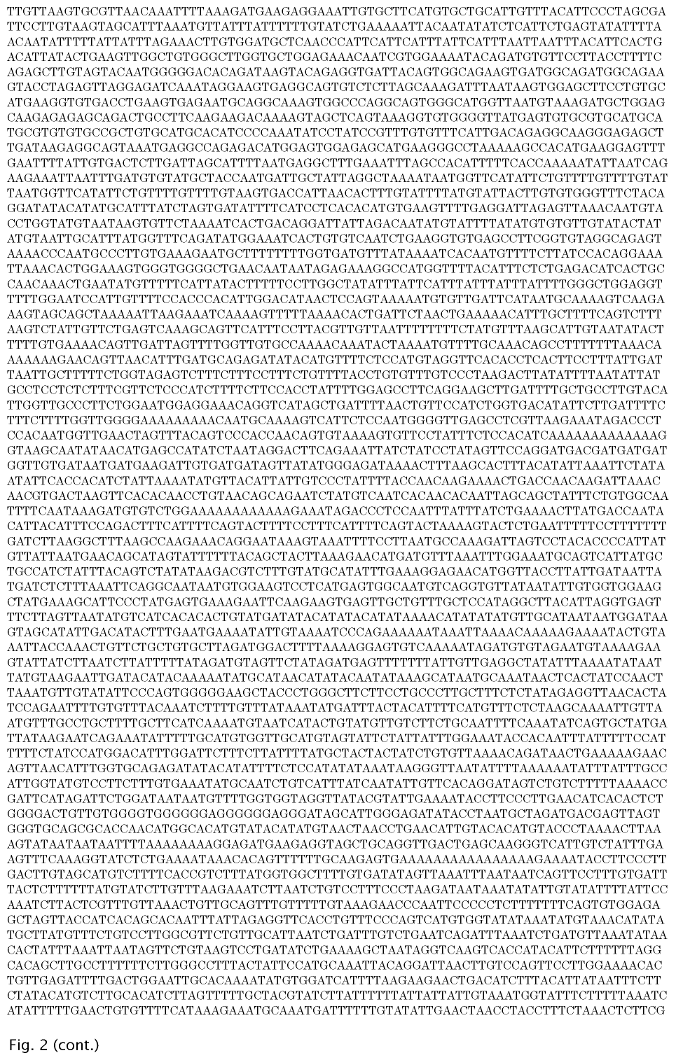

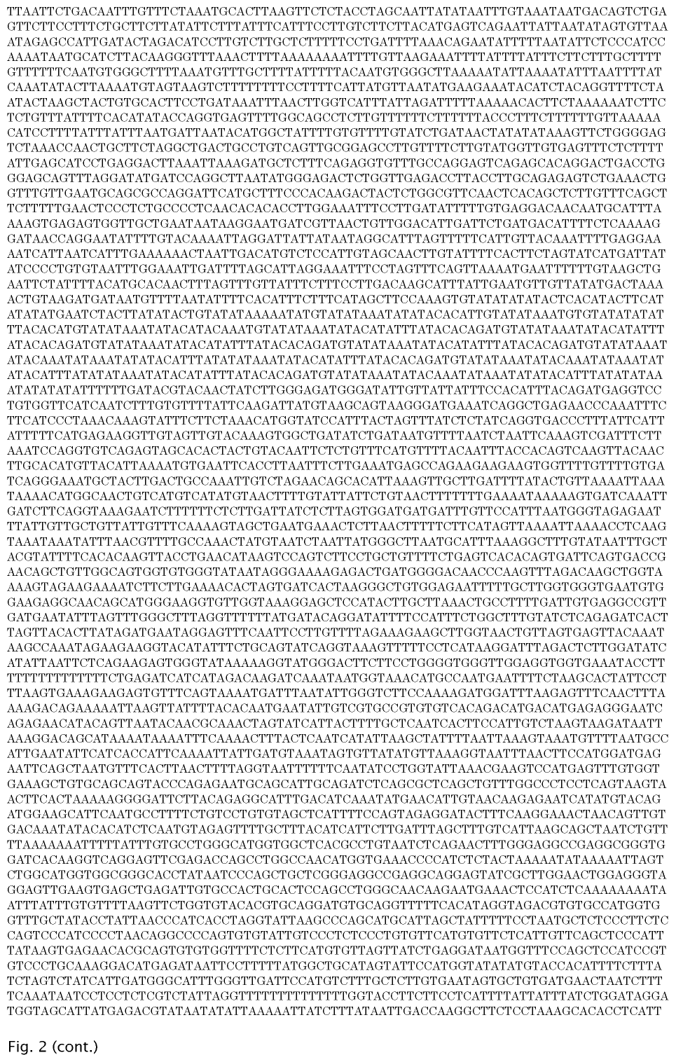

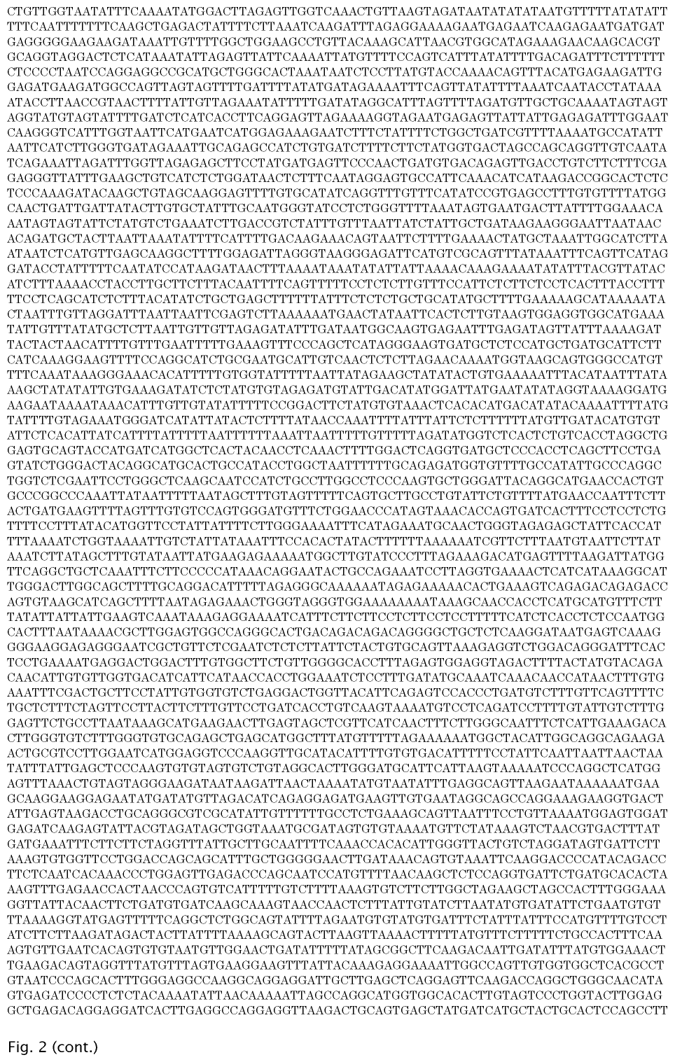

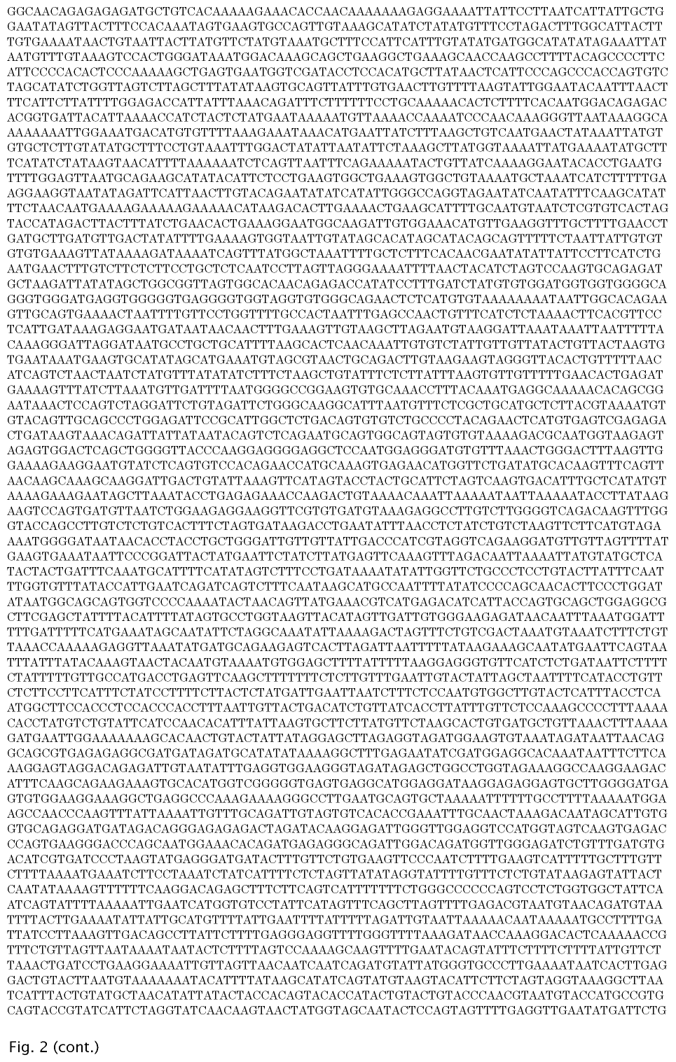

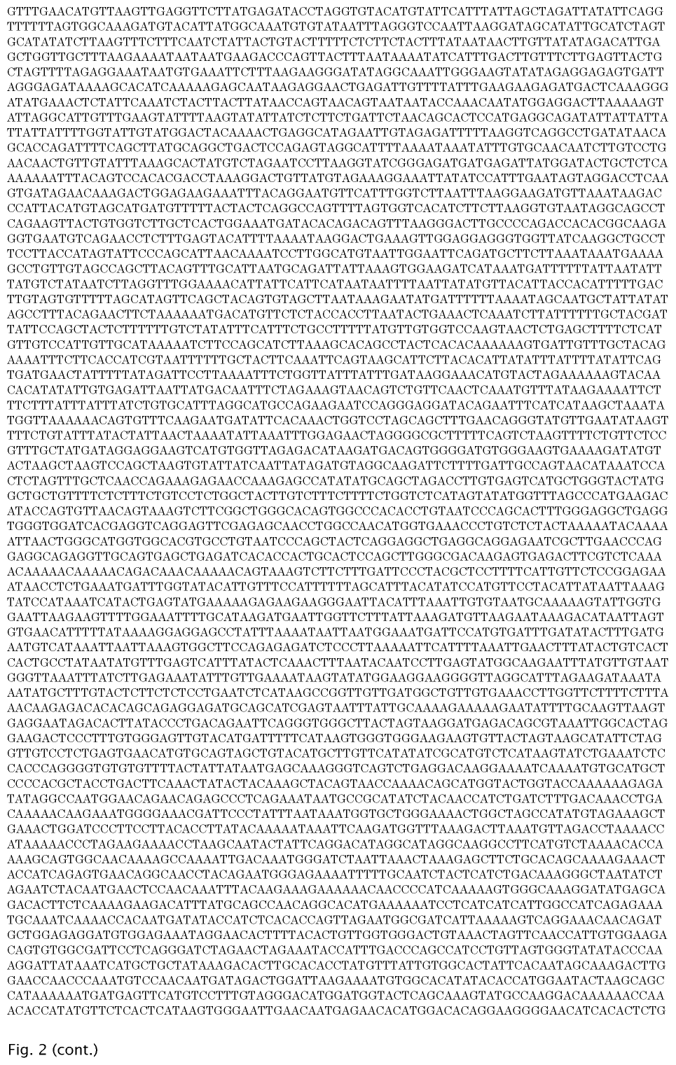

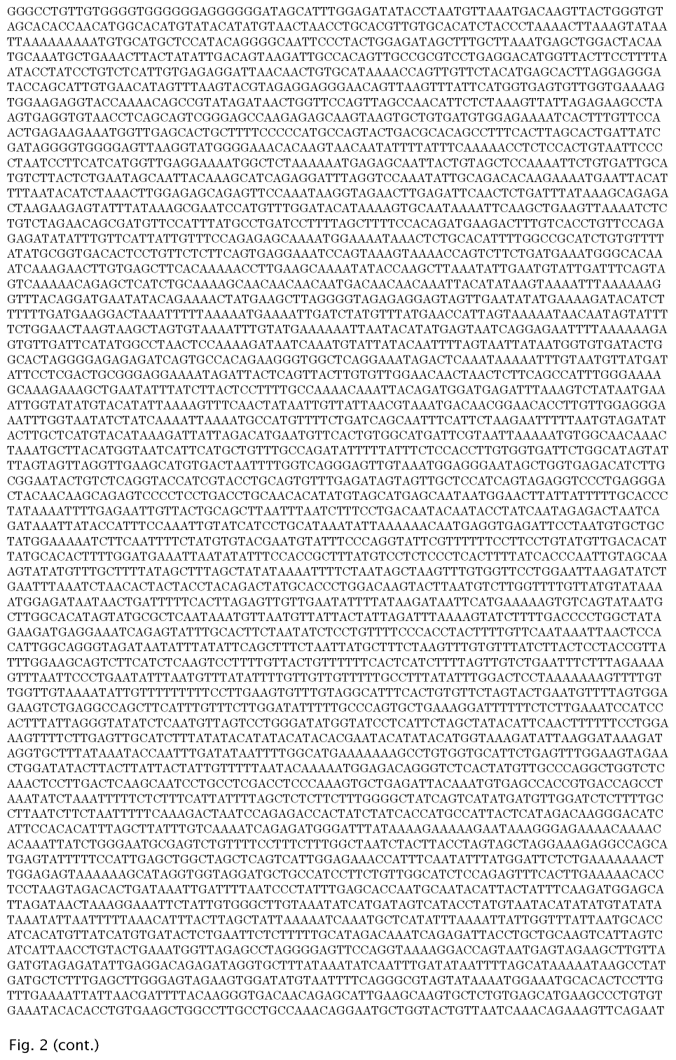

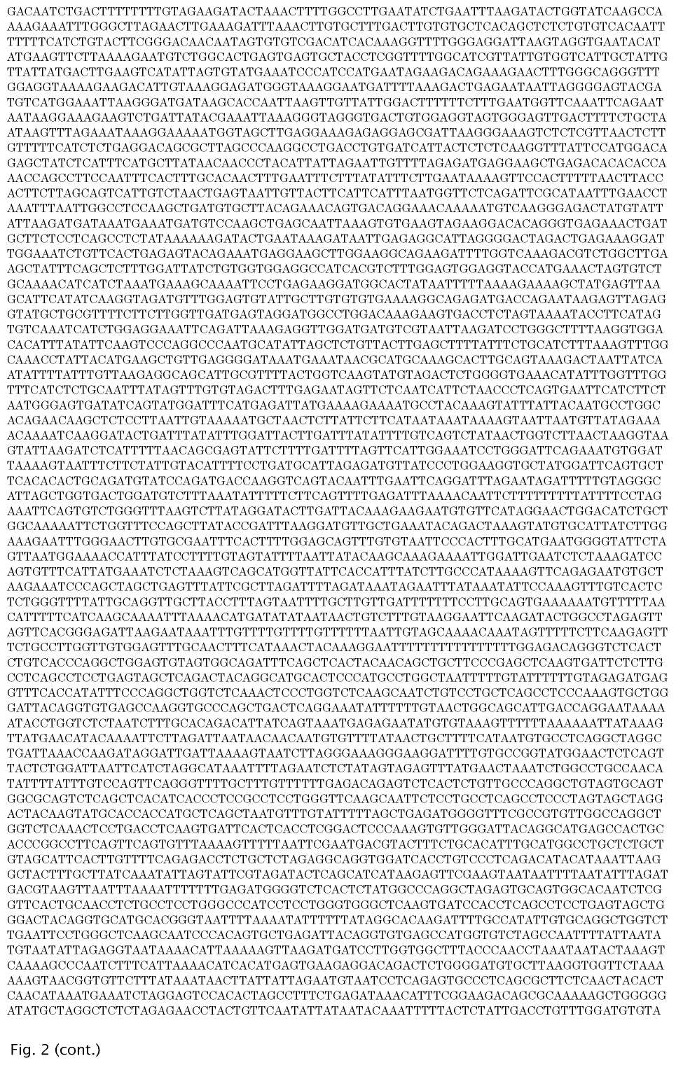

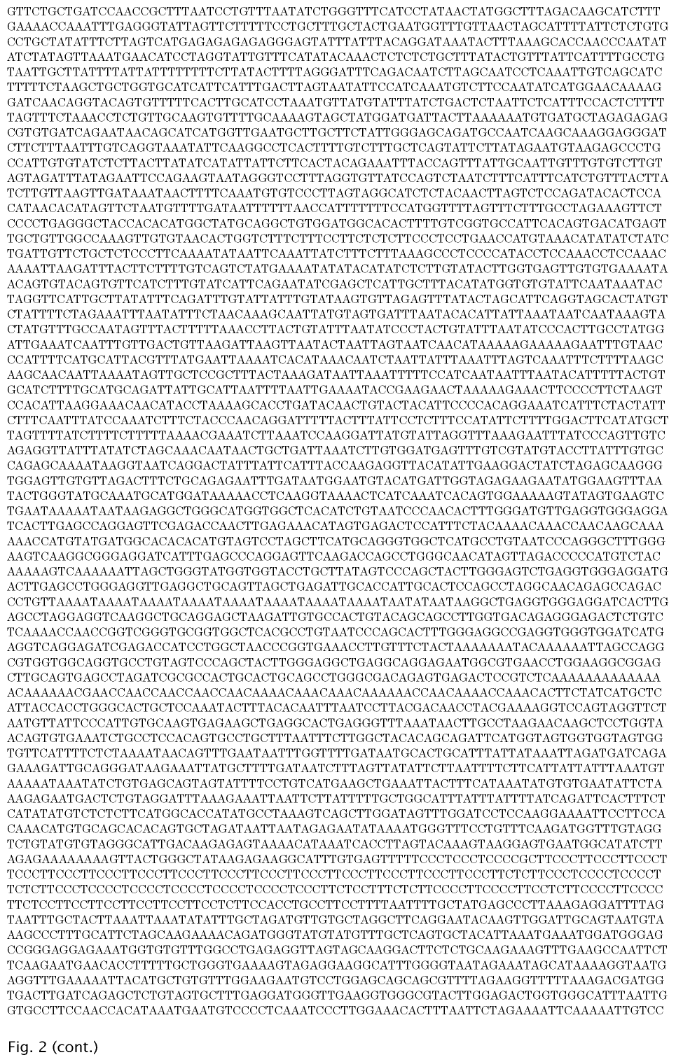

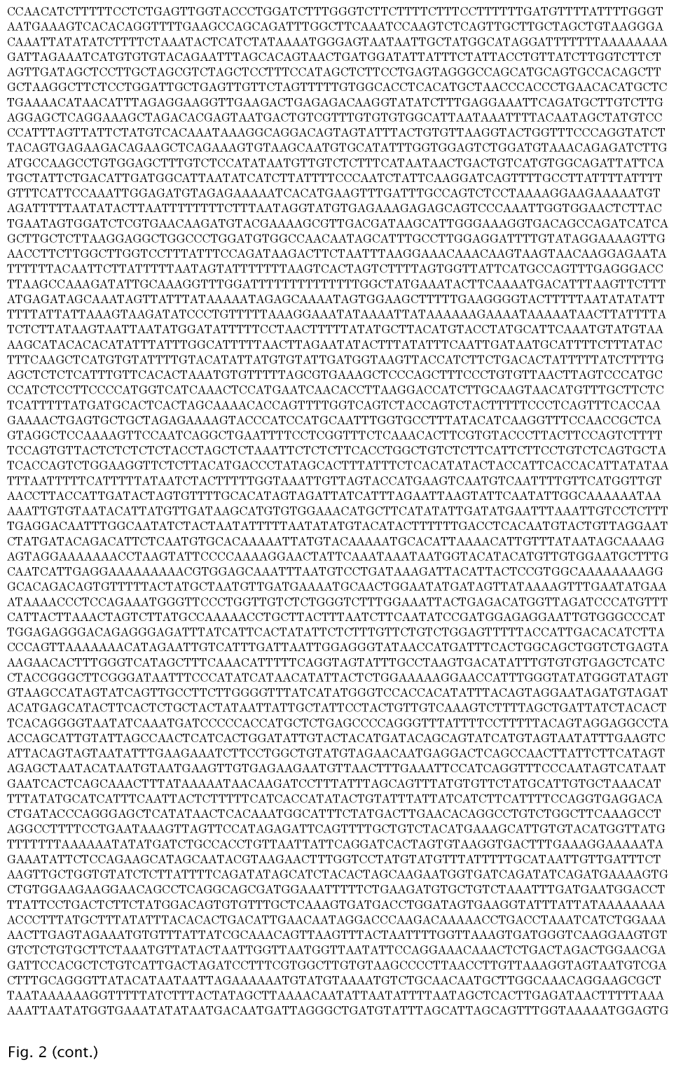

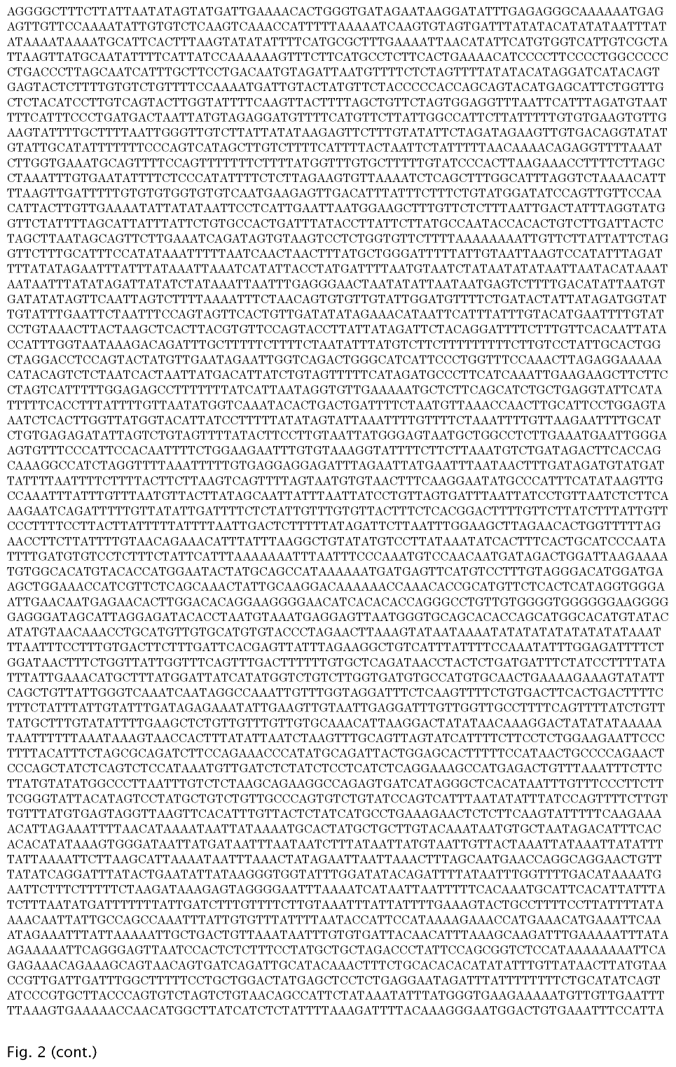

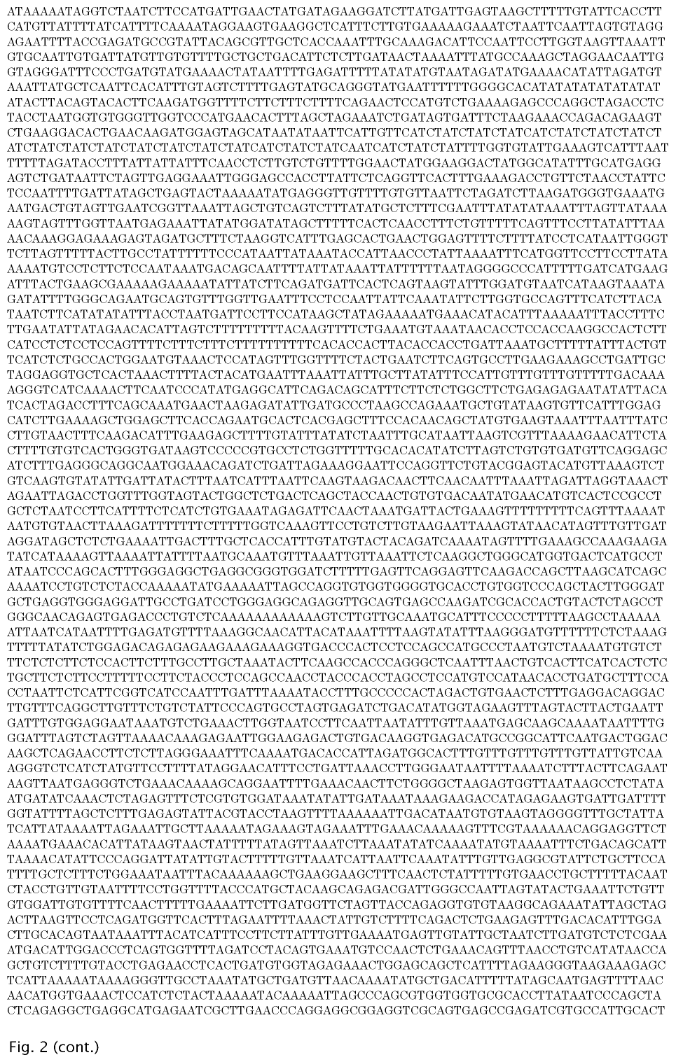

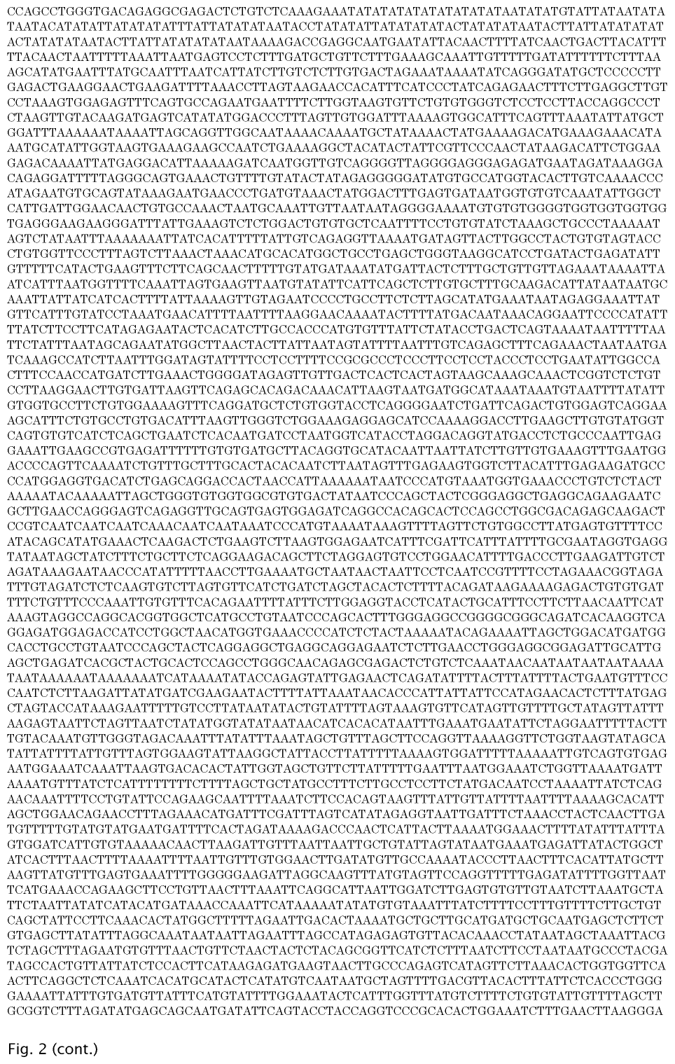

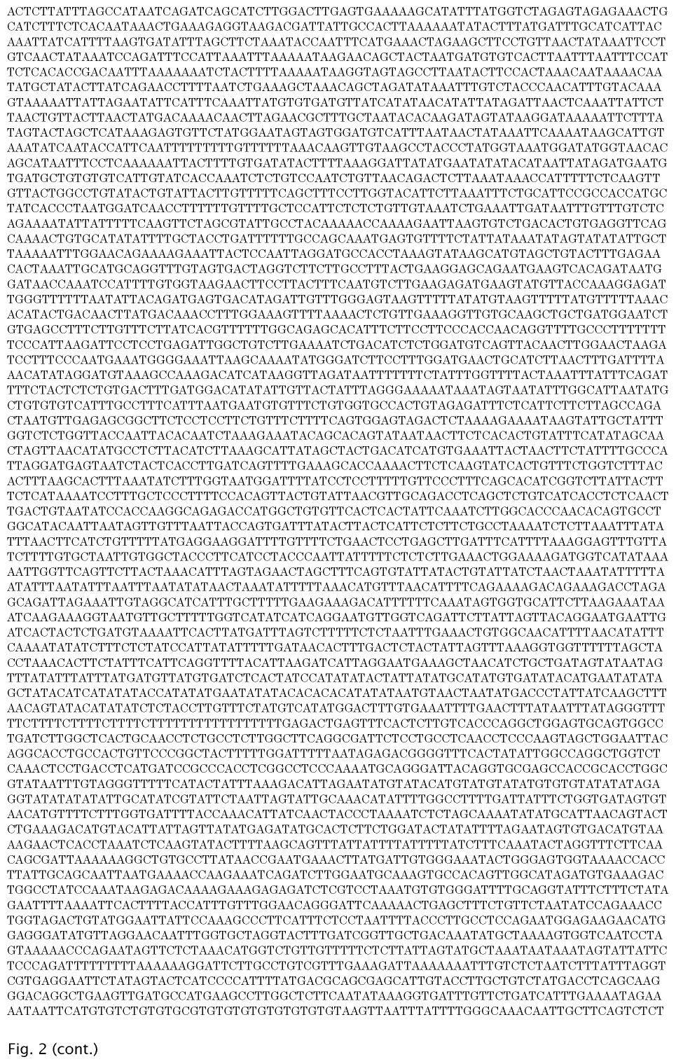

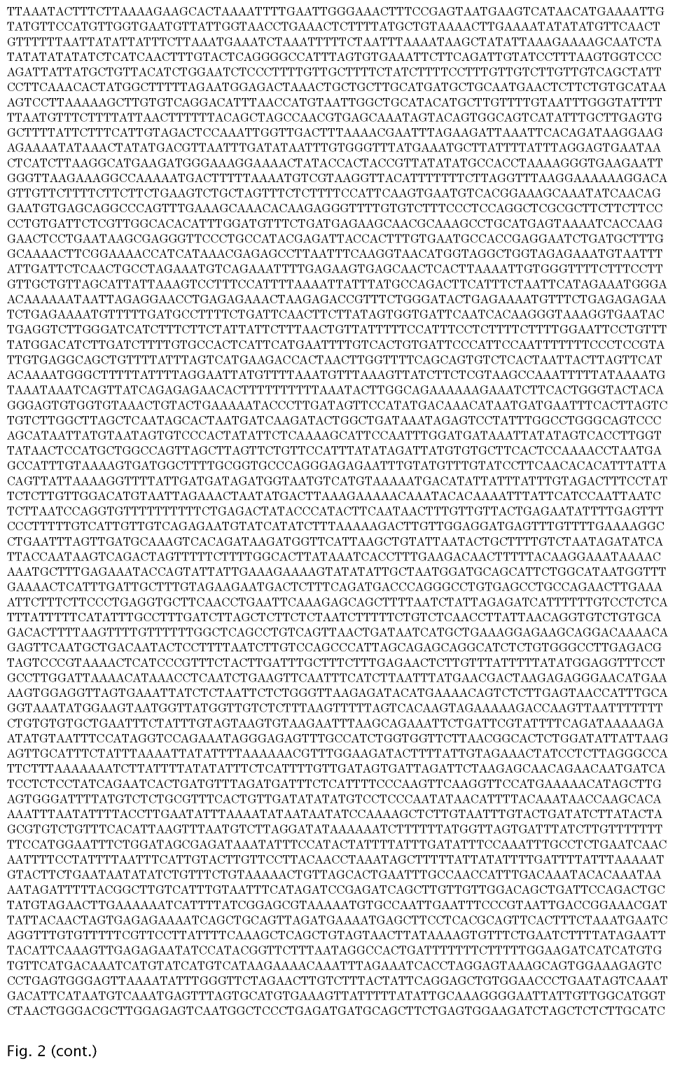

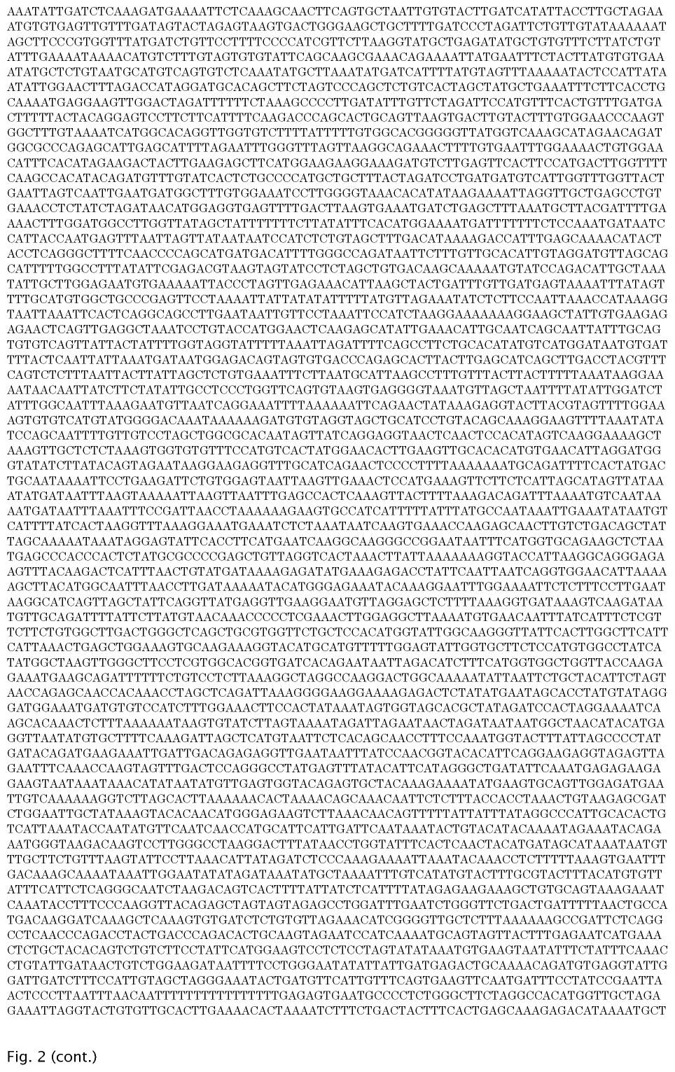

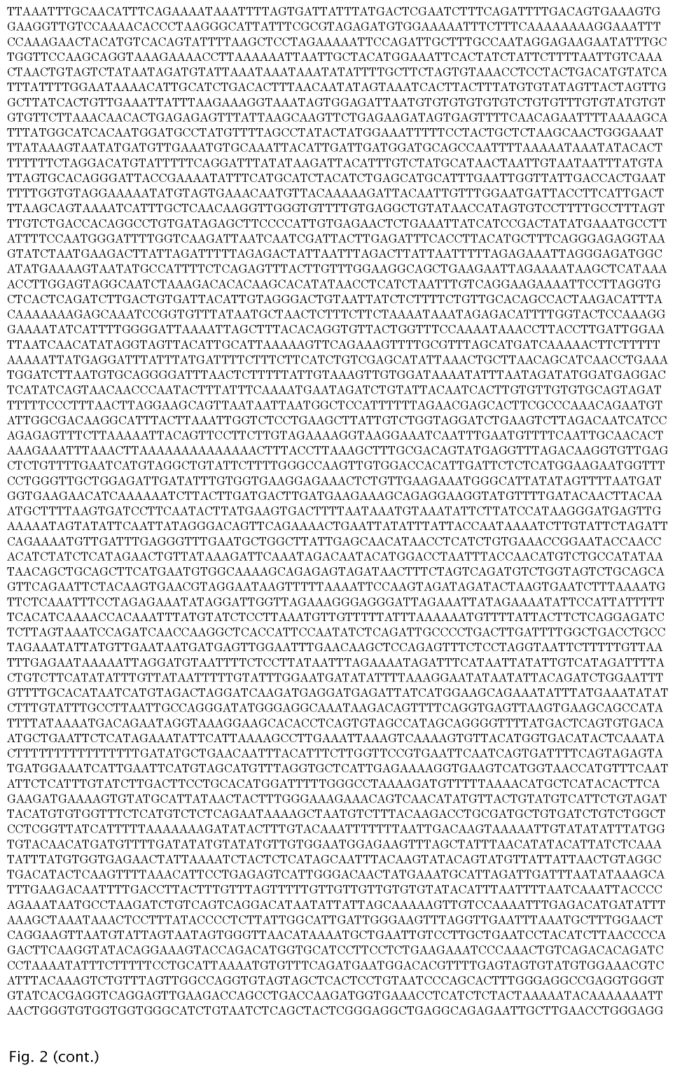

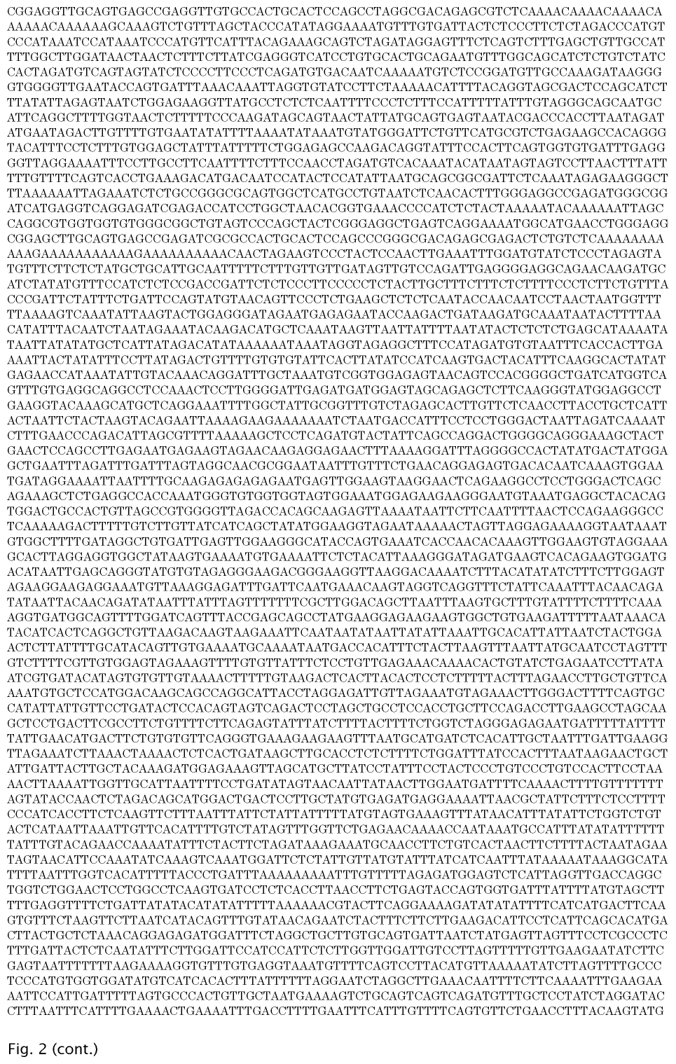

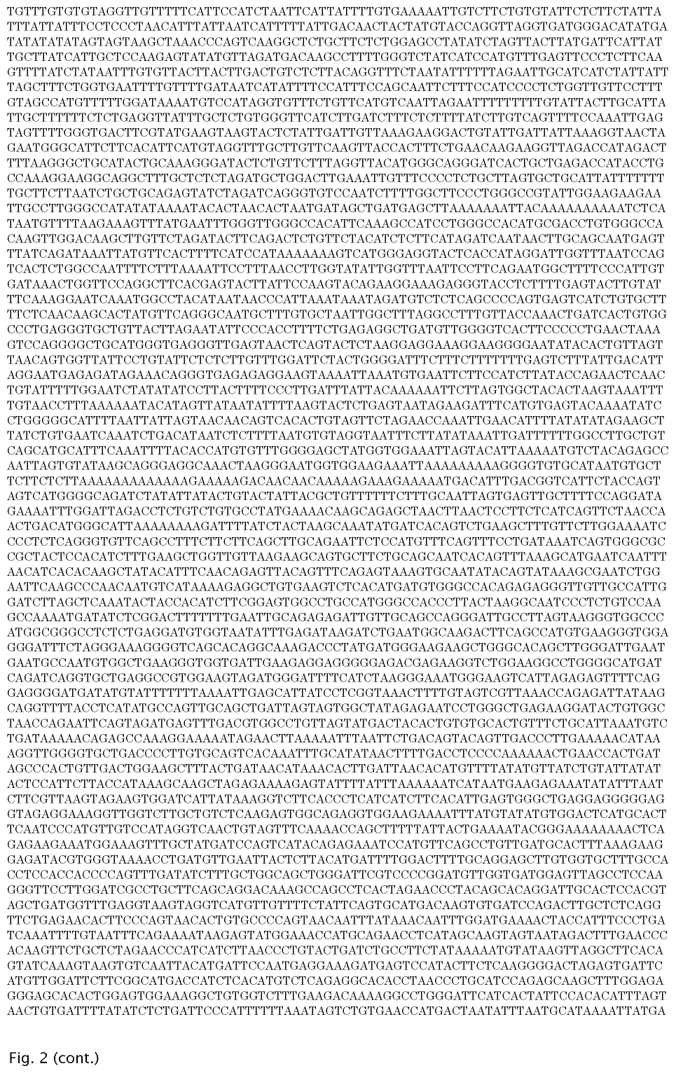

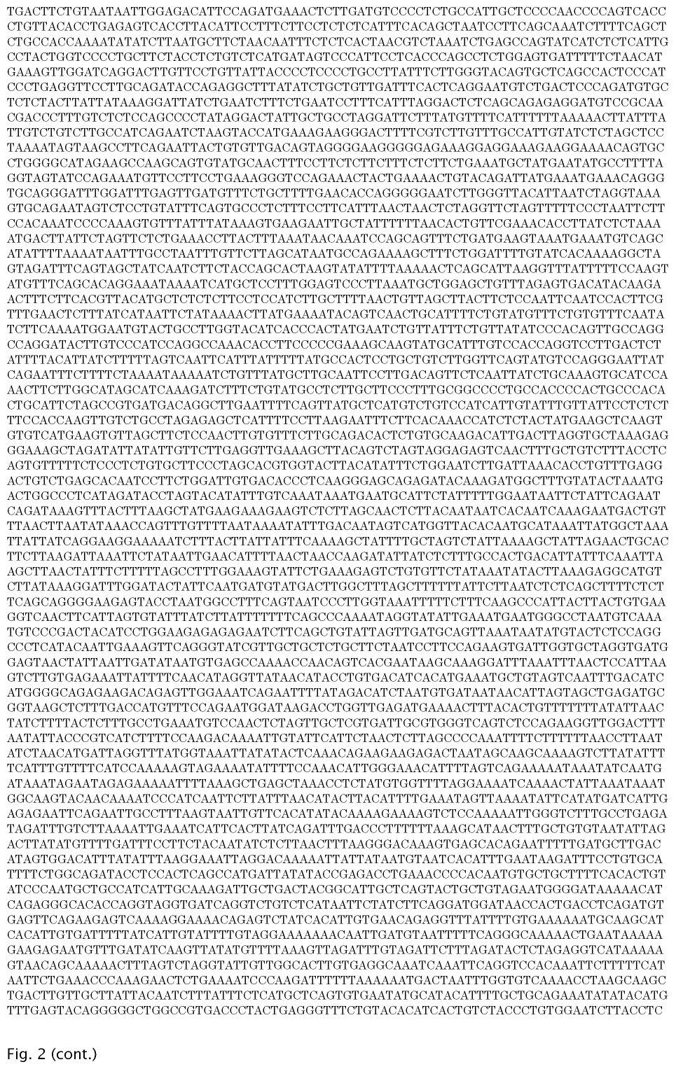

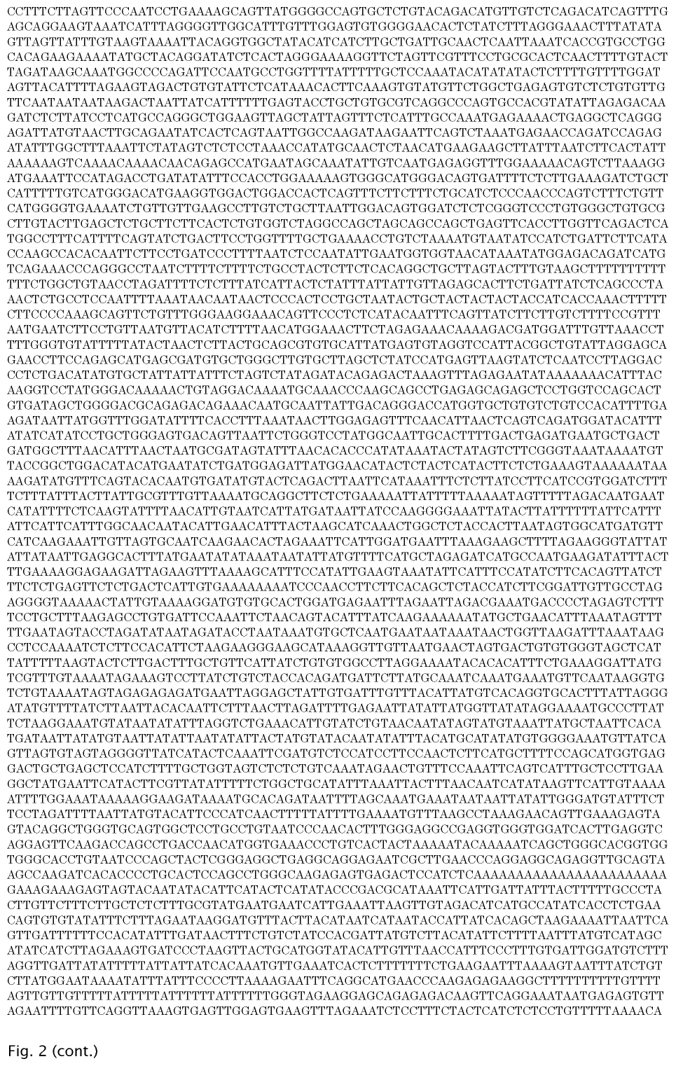

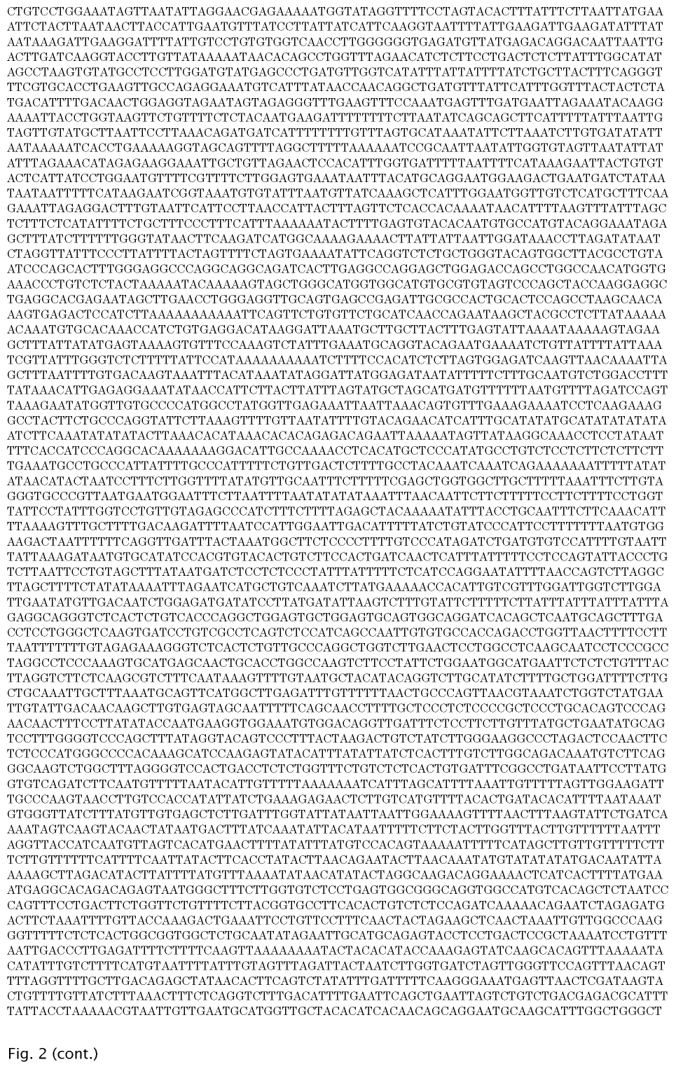

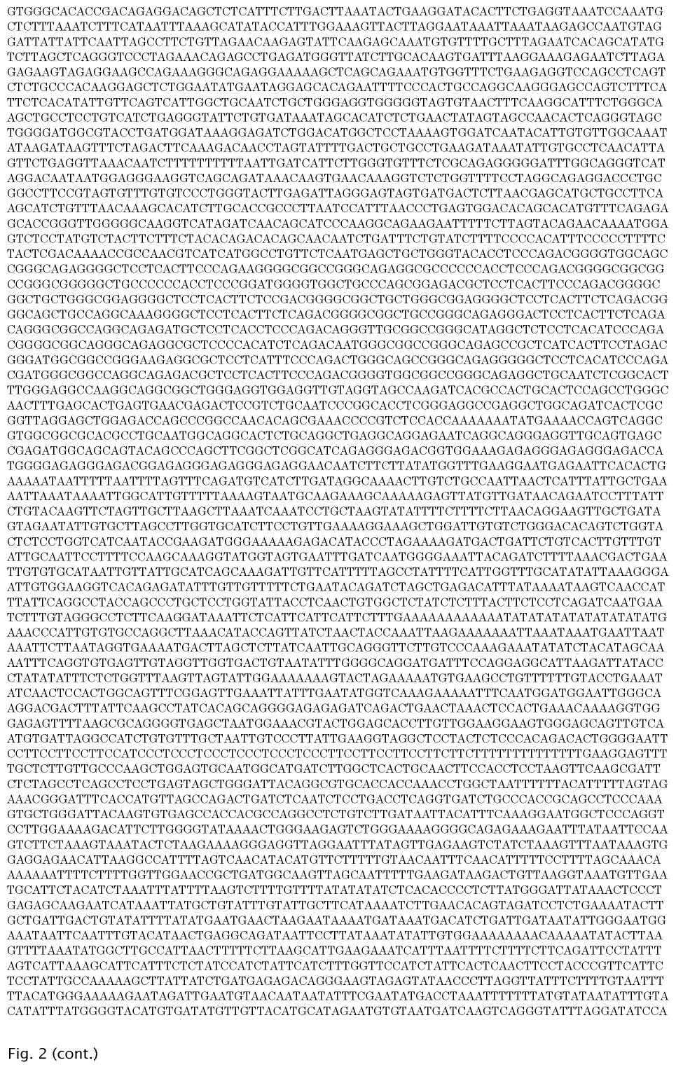

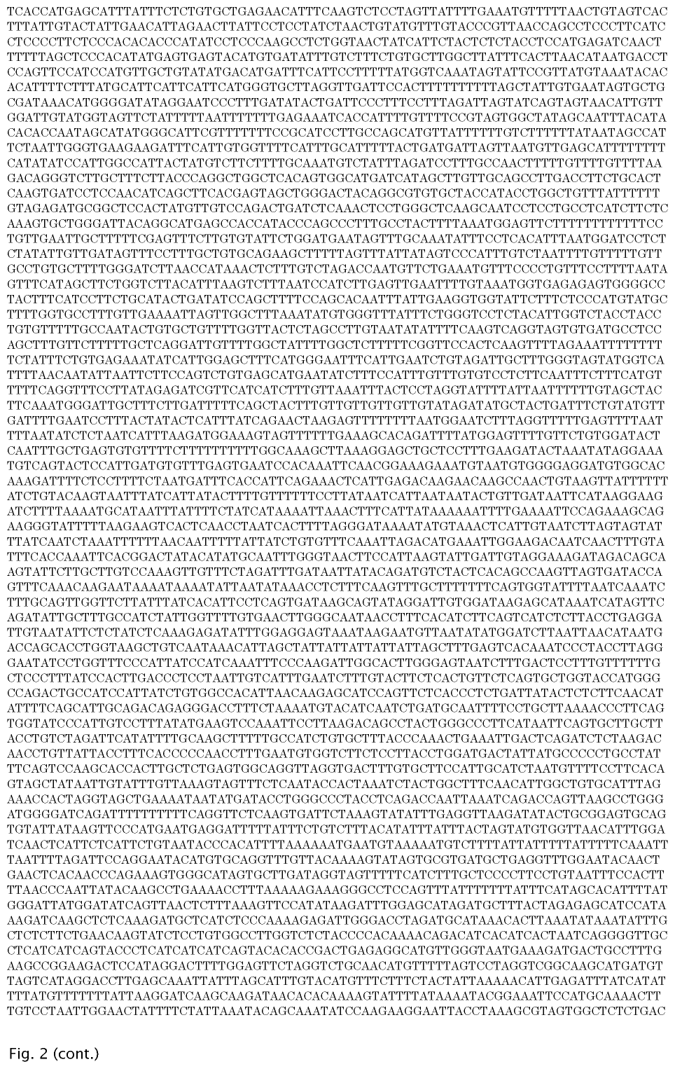

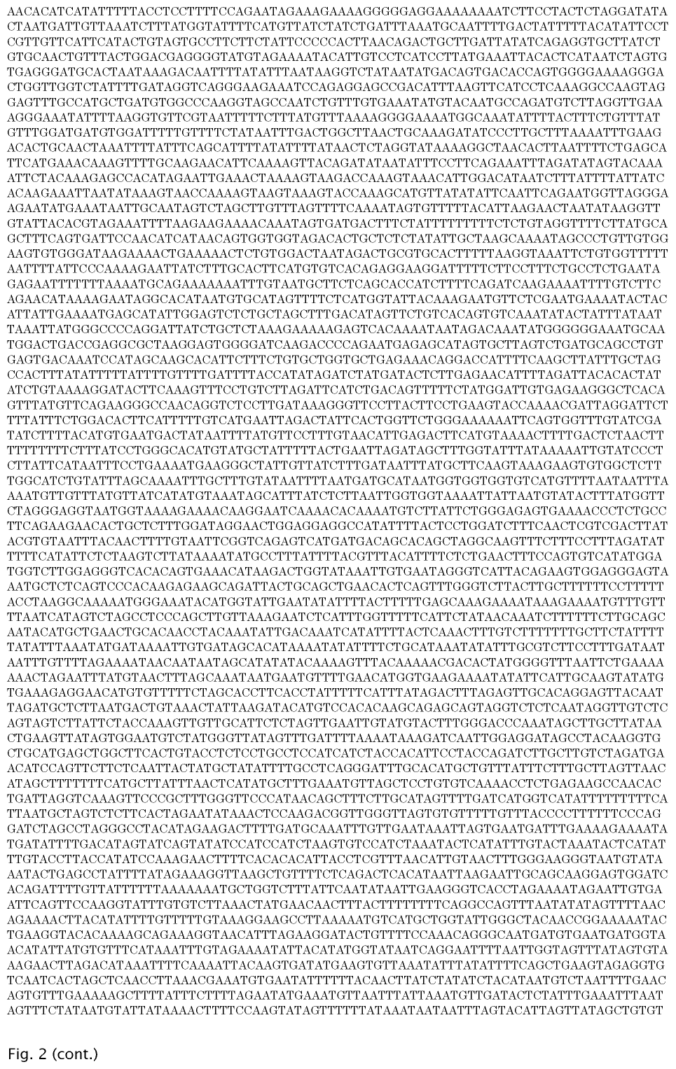

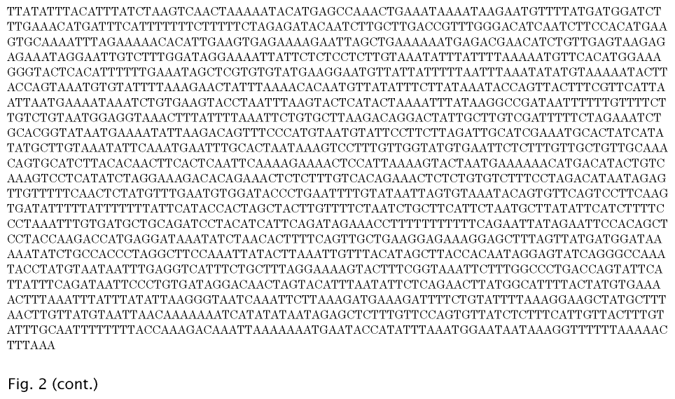

### DETAILED DESCRIPTION

The present invention provides oligonucleotides, which hybridize with mRNA and/or pre-mRNA sequences of the leucine-rich repeat kinase 2 (LRRK2) for example of human origin. These oligonucleotides hybridize with an intron and/or an exon and/or an exon-exon junction and/or an exon-intron junction of the LRRK2 pre-mRNA for example in a neuronal cell or a tumor cell or other mammalian cell. Via recruitment of enzymes such as RNase H the pre mRNA is degraded and the levels of LRRK2 mRNA are reduced. As a consequence the production of LRRK2 protein is prevented and levels of LRRK2 protein are reduced, for example in a neuronal cell or a tumor cell or other mammalian cell. In consequence, the level of LRRK2 protein and its downstream effects decreases. The oligonucleotides of the present invention represent a promising and highly efficient tool for use in methods of preventing and/or treating disorders (e.g., neurodegenerative disorders), where the LRRK2 protein expression, function, and/or activity, respectively, is involved in disease development and progression.

The oligonucleotides of the present invention hybridize, for example, with LRRK2 mRNA of SEQ ID NO. 1 (RefSeq ID NM_198578) and/or LRRK2 pre-mRNA of SEQ ID NO. 2 (GRCh38:12:40224997:40369285).

In the following, the elements of the present invention will be described in more detail. These elements are listed with specific embodiments, however, it should be understood that they may be combined in any manner and in any number to create additional embodiments. The examples and embodiments provided herein are not intended to limit the present invention to only the explicitly described embodiments. This description should be understood to support and encompass embodiments which combine the explicitly described embodiments with any number of the disclosed elements. Furthermore, any permutations or combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Oligonucleotides of the present invention are for example antisense oligonucleotides consisting of or comprising about 10 to about 25 nucleotides, about 10 to about 15 nucleotides, about 15 to about 20 nucleotides, about 12 to about 19 nucleotides, or about 14 to about 18 nucleotides. The oligonucleotides for example consist of or comprise about 10, 11, 12, 13, 14, 15, 16, 17, 18, 19, 20, 21, 22, 23, 24 or 25 nucleotides. The oligonucleotides of the present invention comprise at least one nucleotide which is modified. The modified nucleotide may be, for example, a bridged nucleotide such as a locked nucleic acid (LNA, e.g., 2′,4′-LNA), cET, ENA, a 2′Fluoro modified nucleotide, a 2′O-Methyl modified nucleotide or combinations thereof. The oligonucleotide of the present invention comprises nucleotides having for example one or more, two or more, three or more or four or more of the same or different modifications. Further, the oligonucleotide of the present invention comprises optionally a modified phosphate backbone, wherein the phosphate is, for example, a phosphodiester, a phosphorothioate, methylphosphonate, methyl phosphorothioate, phosphoramidate, phosphorodiamidate, thiophosphoramidate, mesyl phosphoramidate or a combination thereof.

“Reducing” according to the present invention includes inhibiting an effect such as expression in different percentages and amounts, respectively.

The present invention refers to the provision of an oligonucleotide such as an antisense oligonucleotide, mediating the limitation of available LRKK2 mRNA for protein expression. In order to limit protein expression, the oligonucleotide requires the presence of a complementary nucleic acid sequence representing a hybridization target, which allows the formation of heteroduplexes. The oligonucleotides of the present invention hybridize with RNAs of SEQ ID NO. 1 and/or pre-mRNAs of SEQ ID NO. 2. The formation of a heteroduplex between the oligonucleotide and the target RNA leads to enzymes such as RNaseH-mediated degradation or inactivation of the target RNA or termination of transcription, and thus, limits the amount of available LRKK2 mRNA for protein expression.

Further, the present invention relates to an oligonucleotide of the present invention comprising, for example, the one or more, two or more, three or more or four or more modified nucleotide(s) at the 3′- and/or 5′-end of the oligonucleotide and/or at any position within the oligonucleotide, wherein modified nucleotides follow in a row of about 1, 2, 3, 4, 5, or 6 modified nucleotides, or a modified nucleotide is combined with one or more, two or more, three or more or four or more unmodified nucleotides. The following Table 1 presents embodiments of oligonucleotides comprising modified nucleotides, for example, LNA which are indicated by (+) and phosphorothioate (PTO) indicated by (*). The oligonucleotides consisting of or comprising the sequences of Table 1 may comprise any other modified nucleotide and any other combination of modified and unmodified nucleotides. Oligonucleotides of Table 1 hybridize with human LRKK2 mRNA:

TABLE 1
List of human LRRK2-specific ASOs and Control oligonucleotides. An “H” after the ASO name indicates
a human LRRK2-specific sequence that binds to an exonic region of the pre-mRNA and a “Hi” after
the ASO name indicates a human LRRK2-specific sequence that binds to an intronic region of the pre-
mRNA. 16, 17, 18 and 19 mers were designed according to in house criteria, Neg1 (described in
WO2014154843 A1), R01002, R01011 and R01025 were used as non-targeting control oligonucleotides
in some experiments.

| | | | | Position | |
|---|---|---|---|---|---|
| | | | | on | |
| | | | | pre-mRNA | |
| | | | | (GRCh38, | |
| | Antisense | | | Chr 12, | |
| SEQ | Sequence | | Antisense Sequence 5′-3′ | 40224997_ | |
| ID | 5′-3′ | Name | with PTO (*) and LNA (+) | 40369285) | Length |
| 3 | AAGTCACAATCCACA | A57001Hi | +A*+A*+G*T*C*A*C*A*A*T*C*C*A*C*A*G*+ | 509 | 19 |
| | GCAC | | C*+A*+C | | |
| 4 | ACGTCTCCTTCCAAA | A57002Hi | +A*+C*+G*T*C*T*C*C*T*T*C*C*A*A*A*C*+ | 681 | 19 |
| | CAGA | | A*+G*+A | | |
| 5 | AACTAAAGGCTAGAA | A57003Hi | +A*+A*+C*T*A*A*A*G*G*C*T*A*G*A*A*A*+ | 1112 | 19 |
| | ATCT | | T*+C*+T | | |
| 6 | ACTTTCTTTAGATACT | A57004Hi | +A*+C*+T*T*T*C*T*T*T*A*G*A*T*A*C*T*+ | 1142 | 19 |
| | ACA | | A*+C*+A | | |
| 7 | ATCATTCTTAATTACC | A57005Hi | +A*+T*+C*A*T*T*C*T*T*A*A*T*T*A*C*C*+ | 1299 | 19 |
| | ATT | | A*+T*+T | | |
| 8 | AACATTGGCTTTCTG | A57006Hi | +A*+A*+C*A*T*T*G*G*C*T*T*T*C*T*G*A*+ | 1459 | 19 |
| | ACTG | | C*+T*+G | | |
| 9 | TTTATTATGCCTCCAT | A57007Hi | +T*+T*+T*A*T*T*A*T*G*C*C*T*C*C*A*T*+ | 1718 | 19 |
| | CTT | | C*+T*+T | | |
| 10 | TCTTTATTATGCCTCC | A57008Hi | +T*+C*+T*T*T*A*T*T*A*T*G*C*C*T*C*C*+ | 1720 | 19 |
| | ATC | | A*+T*+C | | |
| 11 | TCCGACTTTGAGTCT | A57009Hi | +T*+C*+C*G*A*C*T*T*T*G*A*G*T*C*T*+T* | 1749 | 18 |
| | TAA | | +A*+A | | |
| 12 | GTGACTCATATTCCA | A57010Hi | +G*+T*+G*A*C*T*C*A*T*A*T*T*C*C*A*C*+ | 1773 | 19 |
| | CAGG | | A*+G*+G | | |
| 13 | AATTGATGTCATTGT | A57011Hi | +A*+A*+T*T*G*A*T*G*T*C*A*T*T*G*T*A*+ | 4070 | 19 |
| | ACCT | | C*+C*+T | | |
| 14 | TACTTACAGTAATTAT | A57012Hi | +T*+A*+C*T*T*A*C*A*G*T*A*A*T*T*A*T*+ | 4195 | 19 |
| | ACT | | A*+C*+T | | |
| 15 | CAGGCAACTGAGCTC | A57013Hi | +C*+A*+G*G*C*A*A*C*T*G*A*G*C*T*C*T*+ | 4829 | 19 |
| | TCCA | | C*+C*+A | | |
| 16 | CACATAGGATGCCAA | A57014Hi | +C*+A*+C*A*T*A*G*G*A*T*G*C*C*A*A*A*+ | 4939 | 19 |
| | ACTC | | C*+T*+C | | |
| 17 | TCACATAGGATGCCA | A57015Hi | +T*+C*+A*C*A*T*A*G*G*A*T*G*C*C*A*A*+ | 4940 | 19 |
| | AACT | | A*+C*+T | | |
| 18 | CAATCCAGCTGTCAT | A57016Hi | +C*+A*+A*T*C*C*A*G*C*T*G*T*C*A*T*+A* | 5077 | 18 |
| | AGA | | +G*+A | | |
| 19 | CCACGTAAGCTGAAC | A57017Hi | +C*+C*+A*C*G*T*A*A*G*C*T*G*A*A*C*+A* | 5101 | 18 |
| | AGA | | +G*+A | | |
| 20 | CCTCCACGTAAGCTG | A57018Hi | +C*+C*+T*C*C*A*C*G*T*A*A*G*C*T*G*+A* | 5104 | 18 |
| | AAC | | +A*+C | | |
| 21 | ACATCCTCCTCCACG | A57019Hi | +A*+C*+A*T*C*C*T*C*C*T*C*C*A*C*G*T*+ | 5110 | 19 |
| | TAAG | | A*+A*+G | | |
| 22 | GCTATTTCTACCTATA | A57020Hi | +G*+C*+T*A*T*T*T*C*T*A*C*C*T*A*T*+A* | 5140 | 18 |
| | CT | | +C*+T | | |
| 23 | AAGTTGCTCTTTACC | A57021Hi | +A*+A*+G*T*T*G*C*T*C*T*T*T*A*C*C*T*+ | 5827 | 19 |
| | TCTG | | C*+T*+G | | |
| 24 | GTATATCTTCACTCA | A57022Hi | +G*+T*+A*T*A*T*C*T*T*C*A*C*T*C*A*+G* | 5870 | 18 |
| | GTC | | +T*+C | | |
| 25 | GTAGAACTCCTCAAT | A57023Hi | +G*+T*+A*G*A*A*C*T*C*C*T*C*A*A*T*G*+ | 5996 | 19 |
| | GGCT | | G*+C*+T | | |
| 26 | GGTTGTACCAAGATT | A57024Hi | +G*+G*+T*T*G*T*A*C*C*A*A*G*A*T*T*+A* | 6644 | 18 |
| | ATT | | +T*+T | | |
| 27 | TCAACTTTCAAATTAC | A57025Hi | +T*+C*+A*A*C*T*T*T*C*A*A*A*T*T*A*C*+ | 7773 | 19 |
| | TAC | | T*+A*+C | | |
| 28 | ATTATGCTGCAAACT | A57026Hi | +A*+T*+T*A*T*G*C*T*G*C*A*A*A*C*T*G*+ | 7944 | 19 |
| | GCAA | | C*+A*+A | | |
| 29 | GTTGCATAATCTCCA | A57027Hi | +G*+T*+T*G*C*A*T*A*A*T*C*T*C*C*A*T*+ | 9182 | 19 |
| | TTTG | | T*+T*+G | | |
| 30 | AACGTTGCATAATCT | A57028Hi | +A*+A*+C*G*T*T*G*C*A*T*A*A*T*C*T*C*+ | 9185 | 19 |
| | CCAT | | C*+A*+T | | |
| 31 | TTATTGATTGCTACT | A57029Hi | +T*+T*+A*T*T*G*A*T*T*G*C*T*A*C*T*G*+ | 9717 | 19 |
| | GTTT | | T*+T*+T | | |
| 32 | TATGGCATACTCCAG | A57030Hi | +T*+A*+T*G*G*C*A*T*A*C*T*C*C*A*G*A*+ | 11171 | 19 |
| | AACA | | A*+C*+A | | |
| 33 | ATTATGGCATACTCC | A57031Hi | +A*+T*+T*A*T*G*G*C*A*T*A*C*T*C*C*+A* | 11174 | 18 |
| | AGA | | +G*+A | | |
| 34 | CTCATACTCTCATCTT | A57032Hi | +C*+T*+C*A*T*A*C*T*C*T*C*A*T*C*T*T*+ | 11731 | 19 |
| | CCT | | C*+C*+T | | |
| 35 | CACATTGTCTTCACT | A57033Hi | +C*+A*+C*A*T*T*G*T*C*T*T*C*A*C*T*C*+ | 12157 | 19 |
| | CCAA | | C*+A*+A | | |
| 36 | ACACATCTTGAAGAT | A57034Hi | +A*+C*+A*C*A*T*C*T*T*G*A*A*G*A*T*T*+ | 12449 | 19 |
| | TCCT | | C*+C*+T | | |
| 37 | TCTCCTACACATCTT | A57035Hi | +T*+C*+T*C*C*T*A*C*A*C*A*T*C*T*T*G*+ | 12455 | 19 |
| | GAAG | | A*+A*+G | | |
| 38 | TCTTCATCAATGATA | A57036Hi | +T*+C*+T*T*C*A*T*C*A*A*T*G*A*T*A*C*+ | 12590 | 19 |
| | CCAT | | C*+A*+T | | |
| 39 | CTCTTAGACAACATC | A57037Hi | +C*+T*+C*T*T*A*G*A*C*A*A*C*A*T*C*+T* | 12652 | 18 |
| | TGA | | +G*+A | | |
| 40 | GAAATACATTCTTAT | A57038Hi | +G*+A*+A*A*T*A*C*A*T*T*C*T*T*A*T*G*+ | 12668 | 19 |
| | GCCT | | C*+C*+T | | |
| 41 | CAAGGACTCCAACCA | A57039Hi | +C*+A*+A*G*G*A*C*T*C*C*A*A*C*C*A*A*+ | 13583 | 19 |
| | ACTC | | C*+T*+C | | |
| 42 | AGACTAAACAAGGAC | A57040Hi | +A*+G*+A*C*T*A*A*A*C*A*A*G*G*A*C*T*+ | 13591 | 19 |
| | TCCA | | C*+C*+A | | |
| 43 | TGGTAAACTGATAAC | A57041Hi | +T*+G*+G*T*A*A*A*C*T*G*A*T*A*A*C*T*+ | 13785 | 19 |
| | TGAC | | G*+A*+C | | |
| 44 | TTTCAGTTCAAGAGT | A57042Hi | +T*+T*+T*C*A*G*T*T*C*A*A*G*A*G*T*T*+ | 15055 | 19 |
| | TGTC | | G*+T*+C | | |
| 45 | CGGCTAAATGTTTCA | A57043Hi | +C*+G*+G*C*T*A*A*A*T*G*T*T*T*C*A*G*+ | 15065 | 19 |
| | GTTC | | T*+T*+C | | |
| 46 | TCGGCTAAATGTTTC | A57044Hi | +T*+C*+G*G*C*T*A*A*A*T*G*T*T*T*C*+A* | 15067 | 18 |
| | AGT | | +G*+T | | |
| 47 | ATTGTACTACAAGCT | A57045Hi | +A*+T*+T*G*T*A*C*T*A*C*A*A*G*C*T*C*+ | 15852 | 19 |
| | CTGA | | T*+G*+A | | |
| 48 | TTGCCTCTGTCAATG | A57046Hi | +T*+T*+G*C*C*T*C*T*G*T*C*A*A*T*G*A*+ | 16213 | 19 |
| | AAAC | | A*+A*+C | | |
| 49 | AGTGATTTCCATATC | A57047Hi | +A*+G*+T*G*A*T*T*T*C*C*A*T*A*T*C*T*+ | 16735 | 19 |
| | TGAA | | G*+A*+A | | |
| 50 | TCCTGTTTCTTGGCTT | A57048Hi | +T*+C*+C*T*G*T*T*T*C*T*T*G*G*C*T*T*+ | 18310 | 19 |
| | AAA | | A*+A*+A | | |
| 51 | TGTCAATATGCTACT | A57049Hi | +T*+G*+T*C*A*A*T*A*T*G*C*T*A*C*T*T*+ | 18767 | 19 |
| | TATC | | A*+T*+C | | |
| 52 | GAAACAGGTGAACCT | A57050Hi | +G*+A*+A*A*C*A*G*G*T*G*A*A*C*C*T*+C* | 20304 | 18 |
| | CTA | | +T*+A | | |
| 53 | TTTGTGCAATTCCAG | A57051Hi | +T*+T*+T*G*T*G*C*A*A*T*T*C*C*A*G*T*+ | 20604 | 19 |
| | TCAA | | C*+A*+A | | |
| 54 | TCAGACTGTCATTAT | A57052Hi | +T*+C*+A*G*A*C*T*G*T*C*A*T*T*A*T*T*+ | 20895 | 19 |
| | TTAC | | T*+A*+C | | |
| 55 | AGAACTCAGACTGTC | A57053Hi | +A*+G*+A*A*C*T*C*A*G*A*C*T*G*T*C*A*+ | 20900 | 19 |
| | ATTA | | T*+T*+A | | |
| 56 | TCTTTAATTTAAGTCC | A57054Hi | +T*+C*+T*T*T*A*A*T*T*T*A*A*G*T*C*C*+ | 21645 | 19 |
| | TCA | | T*+C*+A | | |
| 57 | GAGCATCTTTAATTT | A57055Hi | +G*+A*+G*C*A*T*C*T*T*T*A*A*T*T*T*A*+ | 21650 | 19 |
| | AAGT | | A*+G*+T | | |
| 58 | AATATTCCTGGTTAT | A57056Hi | +A*+A*+T*A*T*T*C*C*T*G*G*T*T*A*T*C*+ | 22023 | 19 |
| | CCTT | | C*+T*+T | | |
| 59 | TTACCAGCTTGTCTA | A57057Hi | +T*+T*+A*C*C*A*G*C*T*T*G*T*C*T*A*A*+ | 23835 | 19 |
| | AACT | | A*+C*+T | | |
| 60 | GTGTAACTAAGTGAT | A57058Hi | +G*+T*+G*T*A*A*C*T*A*A*G*T*G*A*T*+C* | 24080 | 18 |
| | CTC | | +T*+C | | |
| 61 | AAGTGTAACTAAGTG | A57059Hi | +A*+A*+G*T*G*T*A*A*C*T*A*A*G*T*G*A*+ | 24081 | 19 |
| | ATCT | | T*+C*+T | | |
| 62 | TTGTCTATGATGATC | A57060Hi | +T*+T*+G*T*C*T*A*T*G*A*T*G*A*T*C*T*+ | 24338 | 19 |
| | TCAG | | C*+A*+G | | |
| 63 | TTACTTAGACAATGG | A57061Hi | +T*+T*+A*C*T*T*A*G*A*C*A*A*T*G*G*A*+ | 24615 | 19 |
| | AAGT | | A*+G*+T | | |
| 64 | ATCTTACTTAGACAA | A57062Hi | +A*+T*+C*T*T*A*C*T*T*A*G*A*C*A*A*T*+ | 24618 | 19 |
| | TGGA | | G*+G*+A | | |
| 65 | ATCTCTCAATAATAA | A57063Hi | +A*+T*+C*T*C*T*C*A*A*T*A*A*T*A*A*C*+ | 26745 | 19 |
| | CTCT | | T*+C*+T | | |
| 66 | GATATTGACAACCTG | A57064Hi | +G*+A*+T*A*T*T*G*A*C*A*A*C*C*T*G*+C* | 26913 | 18 |
| | CTG | | +T*+G | | |
| 67 | AGGTCAACTCTGTCA | A57065Hi | +A*+G*+G*T*C*A*A*C*T*C*T*G*T*C*A*+C* | 26978 | 18 |
| | CAT | | +A*+T | | |
| 68 | ATGACAGCTTCAAAT | A57066Hi | +A*+T*+G*A*C*A*G*C*T*T*C*A*A*A*T*A*+ | 27011 | 19 |
| | AACC | | A*+C*+C | | |
| 69 | AATACTACTATTTGTT | A57067Hi | +A*+A*+T*A*C*T*A*C*T*A*T*T*T*G*T*T*+ | 27238 | 19 |
| | TCC | | T*+C*+C | | |
| 69 | AATACTACTATTTGTT | A575301Hi | +A*+A*T*A*C*T*A*C*T*A*T*T*T*G*T*T*+T* | 27238 | 19 |
| | TCC | | +C*+C | | |
| 69 | AATACTACTATTTGTT | A575302Hi | +A*A*+T*A*C*T*A*C*T*A*T*T*T*G*T*T*+T* | 27238 | 19 |
| | TCC | | +C*+C | | |
| 69 | AATACTACTATTTGTT | A575304Hi | +A*+A*+T*A*C*T*A*C*T*A*T*T*T*G*T*T*+ | 27238 | 19 |
| | TCC | | T*C*+C | | |
| 69 | AATACTACTATTTGTT | A575307Hi | +A*+A*+T*+A*C*T*A*C*T*A*T*T*T*G*T*T* | 27238 | 19 |
| | TCC | | +T*+C*+C | | |
| 69 | AATACTACTATTTGTT | A575308Hi | +A*+A*+T*+A*C*T*A*C*T*A*T*T*T*G*T*T* | 27238 | 19 |
| | TCC | | T*+C*+C | | |
| 69 | AATACTACTATTTGTT | A575309Hi | +A*+A*+T*A*C*T*A*C*T*A*T*T*T*G*T*+T* | 27238 | 19 |
| | TCC | | +T*+C*+C | | |
| 69 | AATACTACTATTTGTT | A575310Hi | +A*+A*T*A*C*T*A*C*T*A*T*T*T*G*T*+T*+ | 27238 | 19 |
| | TCC | | T*+C*+C | | |
| 69 | AATACTACTATTTGTT | A575312Hi | +A*+A*+T*+A*C*T*A*C*T*A*T*T*T*G*T*T* | 27238 | 19 |
| | TCC | | +T*C*+C | | |
| 69 | AATACTACTATTTGTT | A575313Hi | +A*A*+T*A*C*T*A*C*T*A*T*T*T*G*T*+T*+ | 27238 | 19 |
| | TCC | | T*+C*+C | | |
| 70 | CCTTGCTCAACATGA | A57068Hi | +C*+C*+T*T*G*C*T*C*A*A*C*A*T*G*A*G*+ | 27406 | 19 |
| | GATT | | A*+T*+T | | |
| 71 | TTTGAGCAGCCTGAA | A57069Hi | +T*+T*+T*G*A*G*C*A*G*C*C*T*G*A*A*C*+ | 29066 | 19 |
| | CCAT | | C*+A*+T | | |
| 72 | TCTTAACTGCCTCAA | A57070Hi | +T*+C*+T*T*A*A*C*T*G*C*C*T*C*A*A*A*+ | 30217 | 19 |
| | ATAT | | T*+A*+T | | |
| 73 | ACACTGTGATTCAAC | A57071Hi | +A*+C*+A*C*T*G*T*G*A*T*T*C*A*A*C*A*+ | 31035 | 19 |
| | ACTT | | C*+T*+T | | |
| 74 | GATATATTCTGTACA | A57072Hi | +G*+A*+T*A*T*A*T*T*C*T*G*T*A*C*A*A*+ | 32398 | 19 |
| | AGTT | | G*+T*+T | | |
| 75 | TAAGTCTATGGTACT | A57073Hi | +T*+A*+A*G*T*C*T*A*T*G*G*T*A*C*T*+A* | 32529 | 18 |
| | AGT | | +G*+T | | |
| 76 | GTTTCCACAATCTTG | A57074Hi | +G*+T*+T*T*C*C*A*C*A*A*T*C*T*T*G*C*+ | 32569 | 19 |
| | CCAT | | C*+A*+T | | |
| 77 | GTCAACATCAAGCAT | A57075Hi | +G*+T*+C*A*A*C*A*T*C*A*A*G*C*A*T*+C* | 32611 | 18 |
| | CAG | | +A*+G | | |
| 78 | TCTAAGCTTACAACT | A57076Hi | +T*+C*+T*A*A*G*C*T*T*A*C*A*A*C*T*T*+ | 33116 | 19 |
| | TTCA | | T*+C*+A | | |
| 79 | ACATTCTAAGCTTAC | A57077Hi | +A*+C*+A*T*T*C*T*A*A*G*C*T*T*A*C*A*+ | 33120 | 19 |
| | AACT | | A*+C*+T | | |
| 80 | ACATCACTGGACTTC | A57078Hi | +A*+C*+A*T*C*A*C*T*G*G*A*C*T*T*C*T*+ | 34021 | 19 |
| | TTAT | | T*+A*+T | | |
| 81 | GATTAACATCACTGG | A57079Hi | +G*+A*+T*T*A*A*C*A*T*C*A*C*T*G*G*A*+ | 34026 | 19 |
| | ACTT | | C*+T*+T | | |
| 82 | CTTCCTCTTCCAGATT | A57080Hi | +C*+T*+T*C*C*T*C*T*T*C*C*A*G*A*T*T*+ | 34038 | 19 |
| | AAC | | A*+A*+C | | |
| 83 | TAGCTAATAGTACAA | A57081Hi | +T*+A*+G*C*T*A*A*T*A*G*T*A*C*A*A*T*+ | 34942 | 19 |
| | TTCA | | T*+C*+A | | |
| 84 | ACAGTCCTCAAGTGA | A57082Hi | +A*+C*+A*G*T*C*C*T*C*A*A*G*T*G*A*+T* | 36460 | 18 |
| | TTA | | +T*+A | | |
| 85 | ATGCAATATGCTATC | A57083Hi | +A*+T*+G*C*A*A*T*A*T*G*C*T*A*T*C*C*+ | 36843 | 19 |
| | CTTA | | T*+T*+A | | |
| 86 | ACTAGATGCAATATG | A57084Hi | +A*+C*+T*A*G*A*T*G*C*A*A*T*A*T*G*C*+ | 36848 | 19 |
| | CTAT | | T*+A*+T | | |
| 87 | AACTAGCAGTAACTC | A57085Hi | +A*+A*+C*T*A*G*C*A*G*T*A*A*C*T*C*A*+ | 37013 | 19 |
| | AAGA | | A*+G*+A | | |
| 88 | GACCAAATGAACATT | A57086Hi | +G*+A*+C*C*A*A*A*T*G*A*A*C*A*T*T*C*+ | 37688 | 19 |
| | CCTG | | C*+T*+G | | |
| 89 | TCAAAGAGGTTCTGA | A57087Hi | +T*+C*+A*A*A*G*A*G*G*T*T*C*T*G*A*C*+ | 37896 | 19 |
| | CATT | | A*+T*+T | | |
| 90 | ATCTTCCACTTTAATA | A57088Hi | +A*+T*+C*T*T*C*C*A*C*T*T*T*A*A*T*A*+ | 38085 | 19 |
| | ATC | | A*+T*+C | | |
| 91 | TACACTTAGCTGGAC | A57089Hi | +T*+A*+C*A*C*T*T*A*G*C*T*G*G*A*C*+T* | 39087 | 18 |
| | TTA | | +T*+A | | |
| 92 | ATTCTTGCCATACTC | A57090Hi | +A*+T*+T*C*T*T*G*C*C*A*T*A*C*T*C*A*+ | 40151 | 19 |
| | AAGG | | A*+G*+G | | |
| 93 | ACTGTGGCAATCTTA | A57091Hi | +A*+C*+T*G*T*G*G*C*A*A*T*C*T*T*A*+C* | 42242 | 18 |
| | CTG | | +T*+G | | |
| 94 | AGAGTAAGACATGCA | A57092Hi | +A*+G*+A*G*T*A*A*G*A*C*A*T*G*C*A*A*+ | 42832 | 19 |
| | ATCA | | T*+C*+A | | |
| 95 | TCTGCAATATTTGGA | A57093Hi | +T*+C*+T*G*C*A*A*T*A*T*T*T*G*G*A*C*+ | 42880 | 19 |
| | CCTA | | C*+T*+A | | |
| 96 | CTCCAAGTTTAGATG | A57094Hi | +C*+T*+C*C*A*A*G*T*T*T*A*G*A*T*G*T*+ | 42922 | 19 |
| | TATT | | A*+T*+T | | |
| 97 | CTCAAGTTCTACCTT | A57095Hi | +C*+T*+C*A*A*G*T*T*C*T*A*C*C*T*T*A*+ | 42952 | 19 |
| | ATTT | | T*+T*+T | | |
| 98 | ATCTCAAGTTCTACC | A57096Hi | +A*+T*+C*T*C*A*A*G*T*T*C*T*A*C*C*T*+ | 42954 | 19 |
| | TTAT | | T*+A*+T | | |
| 99 | TTGAATCTCAAGTTC | A57097Hi | +T*+T*+G*A*A*T*C*T*C*A*A*G*T*T*C*T*+ | 42958 | 19 |
| | TACC | | A*+C*+C | | |
| 100 | CAGGCATAAATGGAA | A57098Hi | +C*+A*+G*G*C*A*T*A*A*A*T*G*G*A*A*C*+ | 43090 | 19 |
| | CATC | | A*+T*+C | | |
| 101 | ACTGGATTTCCTCAC | A57099Hi | +A*+C*+T*G*G*A*T*T*T*C*C*T*C*A*C*T*+ | 43259 | 19 |
| | TGAA | | G*+A*+A | | |
| 102 | CTTTACTGGATTTCCT | A57100Hi | +C*+T*+T*T*A*C*T*G*G*A*T*T*T*C*C*T*+ | 43263 | 19 |
| | CAC | | C*+A*+C | | |
| 103 | GTTAGTTGTTCCAAC | A57101Hi | +G*+T*+T*A*G*T*T*G*T*T*C*C*A*A*C*+A* | 43904 | 18 |
| | ACA | | +C*+A | | |
| 104 | ATGAGCAAGTATATC | A57102Hi | +A*+T*+G*A*G*C*A*A*G*T*A*T*A*T*C*+T* | 44172 | 18 |
| | TAC | | +A*+C | | |
| 105 | TTCAACCTAACTACT | A57103Hi | +T*+T*+C*A*A*C*C*T*A*A*C*T*A*C*T*A*+ | 44337 | 19 |
| | AAAT | | A*+A*+T | | |
| 106 | ACCACAAACTTAGCT | A57104Hi | +A*+C*+C*A*C*A*A*A*C*T*T*A*G*C*T*A*+ | 44934 | 19 |
| | ATTA | | T*+T*+A | | |
| 107 | CCAGTTCTACTTCCA | A57105Hi | +C*+C*+A*G*T*T*C*T*A*C*T*T*C*C*A*A*+ | 45910 | 19 |
| | AACT | | A*+C*+T | | |
| 108 | GATTGTCATTCTGAA | A57106Hi | +G*+A*+T*T*G*T*C*A*T*T*C*T*G*A*A*C*+ | 47251 | 19 |
| | CTTT | | T*+T*+T | | |
| 109 | TAATTGCTCAGCTTG | A57107Hi | +T*+A*+A*T*T*G*C*T*C*A*G*C*T*T*G*+G* | 48387 | 18 |
| | GAC | | +A*+C | | |
| 110 | CTCTCAGTGAACAGA | A57108Hi | +C*+T*+C*T*C*A*G*T*G*A*A*C*A*G*A*T*+ | 48522 | 19 |
| | TTTC | | T*+T*+C | | |
| 111 | TTTCTGTACTCTCAGT | A57109Hi | +T*+T*+T*C*T*G*T*A*C*T*C*T*C*A*G*T*+ | 48530 | 19 |
| | GAA | | G*+A*+A | | |
| 112 | AGATAATCCAAAGAG | A57110Hi | +A*+G*+A*T*A*A*T*C*C*A*A*A*G*A*G*C*+ | 48605 | 19 |
| | CTGA | | T*+G*+A | | |
| 113 | CTCCACTCCAAAGAC | A57111Hi | +C*+T*+C*C*A*C*T*C*C*A*A*A*G*A*C*+G* | 48638 | 18 |
| | GTG | | +T*+G | | |
| 114 | CTAACTCTTATTCTG | A57112Hi | +C*+T*+A*A*C*T*C*T*T*A*T*T*C*T*G*+G* | 48812 | 18 |
| | GTC | | +T*+C | | |
| 115 | TCTAACTCTTATTCTG | A57113Hi | +T*+C*+T*A*A*C*T*C*T*T*A*T*T*C*T*+G* | 48813 | 18 |
| | GT | | +G*+T | | |
| 116 | CCATCCTACTCATCA | A57114Hi | +C*+C*+A*T*C*C*T*A*C*T*C*A*T*C*A*A*+ | 48851 | 19 |
| | ACCA | | C*+C*+A | | |
| 117 | CCATCCTACTCATCA | A57115Hi | +C*+C*+A*T*C*C*T*A*C*T*C*A*T*C*A*+A* | 48852 | 18 |
| | ACC | | +C*+C | | |
| 118 | ATCCAACCTCTTTAAT | A57116Hi | +A*+T*+C*C*A*A*C*C*T*C*T*T*T*A*A*T*+ | 48933 | 19 |
| | CTG | | C*+T*+G | | |
| 119 | CATCCAACCTCTTTA | A57117Hi | +C*+A*+T*C*C*A*A*C*C*T*C*T*T*T*A*A*+ | 48934 | 19 |
| | ATCT | | T*+C*+T | | |
| 120 | GAGATCTTAATACTT | A57118Hi | +G*+A*+G*A*T*C*T*T*A*A*T*A*C*T*T*+A* | 49537 | 18 |
| | ACC | | +C*+C | | |
| 121 | TCCAAGATAATGCAC | A57119Hi | +T*+C*+C*A*A*G*A*T*A*A*T*G*C*A*C*A*+ | 49998 | 19 |
| | ATAC | | T*+A*+C | | |
| 122 | AGATGAATTAATCCA | A57120Hi | +A*+G*+A*T*G*A*A*T*T*A*A*T*C*C*A*G*+ | 51201 | 19 |
| | GAGT | | A*+G*+T | | |
| 123 | TAGAACCACCTTAAG | A57121Hi | +T*+A*+G*A*A*C*C*A*C*C*T*T*A*A*G*+C* | 52279 | 18 |
| | CAC | | +A*+C | | |
| 124 | TCACACAACTCACCA | A57122Hi | +T*+C*+A*C*A*C*A*A*C*T*C*A*C*C*A*A*+ | 53941 | 19 |
| | AGTA | | G*+T*+A | | |
| 125 | TCTGGCACAAATAAG | A57123Hi | +T*+C*+T*G*G*C*A*C*A*A*A*T*A*A*G*G*+ | 54906 | 19 |
| | GTAC | | T*+A*+C | | |
| 126 | TTTCCACTGTGATTT | A57124Hi | +T*+T*+T*C*C*A*C*T*G*T*G*A*T*T*T*G*+ | 55121 | 19 |
| | GATG | | A*+T*+G | | |
| 127 | TTCAGACTTCACTAT | A57125Hi | +T*+T*+C*A*G*A*C*T*T*C*A*C*T*A*T*A*+ | 55140 | 19 |
| | ACTT | | C*+T*+T | | |
| 128 | CACCACTACCATGAA | A57126Hi | +C*+A*+C*C*A*C*T*A*C*C*A*T*G*A*A*+T* | 56373 | 18 |
| | TCT | | +C*+T | | |
| 129 | TACCACCACTACCAT | A57127Hi | +T*+A*+C*C*A*C*C*A*C*T*A*C*C*A*T*G*+ | 56375 | 19 |
| | GAAT | | A*+A*+T | | |
| 130 | CTTTAAATCCTACAG | A57128Hi | +C*+T*+T*T*A*A*A*T*C*C*T*A*C*A*G*A*+ | 56645 | 19 |
| | AGTC | | G*+T*+C | | |
| 131 | AAGCTGACTTTAGGC | A57129Hi | +A*+A*+G*C*T*G*A*C*T*T*T*A*G*G*C*A*+ | 56742 | 19 |
| | ATAT | | T*+A*+T | | |
| 132 | ACTATCCAAGCTGAC | A57130Hi | +A*+C*+T*A*T*C*C*A*A*G*C*T*G*A*C*T*+ | 56749 | 19 |
| | TTTA | | T*+T*+A | | |
| 133 | TCCTCAAAGATATAC | A57131Hi | +T*+C*+C*T*C*A*A*A*G*A*T*A*T*A*C*C*+ | 58333 | 19 |
| | CTTG | | T*+T*+G | | |
| 134 | CAAGATCTCTGTTTA | A57132Hi | +C*+A*+A*G*A*T*C*T*C*T*G*T*T*T*A*C*+ | 58587 | 19 |
| | CATC | | A*+T*+C | | |
| 135 | ATCCAAACCTTTGCA | A57133Hi | +A*+T*+C*C*A*A*A*C*C*T*T*T*G*C*A*A*+ | 59248 | 19 |
| | ATAT | | T*+A*+T | | |
| 136 | TAGATTCCTAACAGT | A57134Hi | +T*+A*+G*A*T*T*C*C*T*A*A*C*A*G*T*+A* | 60574 | 18 |
| | ACA | | +C*+A | | |
| 137 | AAGTGTTCTTTACTC | A57135Hi | +A*+A*+G*T*G*T*T*C*T*T*T*A*C*T*C*A*+ | 61211 | 19 |
| | AGAC | | G*+A*+C | | |
| 138 | ATTCTTCTCACAACTT | A57136Hi | +A*+T*+T*C*T*T*C*T*C*A*C*A*A*C*T*T*+ | 61793 | 19 |
| | CAT | | C*+A*+T | | |
| 139 | CTCTCAAATATCCTTA | A57137Hi | +C*+T*+C*T*C*A*A*A*T*A*T*C*C*T*T*A*+ | 63162 | 19 |
| | TTC | | T*+T*+C | | |
| 140 | GTATAAATCAGTGGC | A57138Hi | +G*+T*+A*T*A*A*A*T*C*A*G*T*G*G*C*+A* | 64255 | 18 |
| | ACA | | +C*+A | | |
| 141 | AACATCCAATACAAC | A57139Hi | +A*+A*+C*A*T*C*C*A*A*T*A*C*A*A*C*A*+ | 64669 | 19 |
| | ACAC | | C*+A*+C | | |
| 142 | TGAAATGTCTATTAG | A57140Hi | +T*+G*+A*A*A*T*G*T*C*T*A*T*T*A*G*C*+ | 67470 | 19 |
| | CACA | | A*+C*+A | | |
| 142 | TGAAATGTCTATTAG | A575315Hi | +T*+G*A*A*A*T*G*T*C*T*A*T*T*A*G*C*+A | 67470 | 19 |
| | CACA | | *+C*+A | | |
| 142 | TGAAATGTCTATTAG | A575316Hi | +T*G*+A*A*A*T*G*T*C*T*A*T*T*A*G*C*+A | 67470 | 19 |
| | CACA | | *+C*+A | | |
| 142 | TGAAATGTCTATTAG | A575318Hi | +T*+G*+A*A*A*T*G*T*C*T*A*T*T*A*G*C*+ | 67470 | 19 |
| | CACA | | A*C*+A | | |
| 142 | TGAAATGTCTATTAG | A575321Hi | +T*+G*+A*+A*A*T*G*T*C*T*A*T*T*A*G*C* | 67470 | 19 |
| | CACA | | +A*+C*+A | | |
| 142 | TGAAATGTCTATTAG | A575322Hi | +T*+G*+A*+A*A*T*G*T*C*T*A*T*T*A*G*C* | 67470 | 19 |
| | CACA | | A*+C*+A | | |
| 142 | TGAAATGTCTATTAG | A575323Hi | +T*+G*+A*A*A*T*G*T*C*T*A*T*T*A*G*+C* | 67470 | 19 |
| | CACA | | +A*+C*+A | | |
| 142 | TGAAATGTCTATTAG | A575324Hi | +T*+G*A*A*A*T*G*T*C*T*A*T*T*A*G*+C*+ | 67470 | 19 |
| | CACA | | A*+C*+A | | |
| 142 | TGAAATGTCTATTAG | A575326Hi | +T*+G*+A*+A*A*T*G*T*C*T*A*T*T*A*G*C* | 67470 | 19 |
| | CACA | | +A*C*+A | | |
| 142 | TGAAATGTCTATTAG | A575327Hi | +T*G*+A*A*A*T*G*T*C*T*A*T*T*A*G*+C*+ | 67470 | 19 |
| | CACA | | A*+C*+A | | |
| 143 | CCTACTCTTTATCTTA | A57141Hi | +C*+C*+T*A*C*T*C*T*T*T*A*T*C*T*T*A*+ | 67740 | 19 |
| | GAA | | G*+A*+A | | |
| 144 | TACTCAATCATAAGA | A57142Hi | +T*+A*+C*T*C*A*A*T*C*A*T*A*A*G*A*+T* | 68485 | 18 |
| | TCC | | +C*+C | | |
| 145 | GATTTCTAGCTAAAG | A57143Hi | +G*+A*+T*T*T*C*T*A*G*C*T*A*A*A*G*T*+ | 69023 | 19 |
| | TGTT | | G*+T*+T | | |
| 146 | ACATCACACAGACTA | A57144Hi | +A*+C*+A*T*C*A*C*A*C*A*G*A*C*T*A*A*+ | 70797 | 19 |
| | AGAT | | G*+A*+T | | |
| 147 | TACCAAACCAGGTCT | A57145Hi | +T*+A*+C*C*A*A*A*C*C*A*G*G*T*C*T*A*+ | 70984 | 19 |
| | AATT | | A*+T*+T | | |
| 148 | CAGAGCCAGTACTAC | A57146Hi | +C*+A*+G*A*G*C*C*A*G*T*A*C*T*A*C*C*+ | 70996 | 19 |
| | CAAA | | A*+A*+A | | |
| 149 | TTTGATCTGTAGTAC | A57147Hi | +T*+T*+T*G*A*T*C*T*G*T*A*G*T*A*C*A*+ | 71257 | 19 |
| | ATAC | | T*+A*+C | | |
| 150 | AAGCCTGAAACAAGT | A57148Hi | +A*+A*+G*C*C*T*G*A*A*A*C*A*A*G*T*C*+ | 72080 | 19 |
| | CCTG | | C*+T*+G | | |
| 151 | TTTACTGAGTCAGGT | A57149Hi | +T*+T*+T*A*C*T*G*A*G*T*C*A*G*G*T*+A* | 75451 | 18 |
| | ATA | | +T*+A | | |
| 152 | GATCATTATTAGTTTC | A57150Hi | +G*+A*+T*C*A*T*T*A*T*T*A*G*T*T*T*C*+ | 75543 | 19 |
| | TGA | | T*+G*+A | | |
| 153 | ATGGCTTTGATCATT | A57151Hi | +A*+T*+G*G*C*T*T*T*G*A*T*C*A*T*T*A*+ | 75551 | 19 |
| | ATTA | | T*+T*+A | | |
| 154 | AACTTAATCACAAGT | A57152Hi | +A*+A*+C*T*T*A*A*T*C*A*C*A*A*G*T*T*+ | 75722 | 19 |
| | TCCT | | C*+C*+T | | |
| 155 | ACAGAGCATCCTGAA | A57153Hi | +A*+C*+A*G*A*G*C*A*T*C*C*T*G*A*A*A*+ | 75814 | 19 |
| | ACTT | | C*+T*+T | | |
| 156 | TCTCAAATGTAAGAC | A57154Hi | +T*+C*+T*C*A*A*A*T*G*T*A*A*G*A*C*C*+ | 76162 | 19 |
| | CACT | | A*+C*+T | | |
| 157 | GATTCTCCACTTAAG | A57155Hi | +G*+A*+T*T*C*T*C*C*A*C*T*T*A*A*G*A*+ | 76526 | 19 |
| | ACTT | | C*+T*+T | | |
| 158 | TTTATCTAGACAATCT | A57156Hi | +T*+T*+T*A*T*C*T*A*G*A*C*A*A*T*C*T*+ | 76645 | 19 |
| | TCA | | T*+C*+A | | |
| 159 | GTTTCTAAAGGTTCT | A57157Hi | +G*+T*+T*T*C*T*A*A*A*G*G*T*T*C*T*G*+ | 77928 | 19 |
| | GTTC | | T*+T*+C | | |
| 160 | TAGCCAGTATAATCT | A57158Hi | +T*+A*+G*C*C*A*G*T*A*T*A*A*T*C*T*C*+ | 78143 | 19 |
| | CATT | | A*+T*+T | | |
| 161 | TGAGCTAGTACTATA | A57159Hi | +T*+G*+A*G*C*T*A*G*T*A*C*T*A*T*A*A*+ | 79662 | 19 |
| | AAGA | | A*+G*+A | | |
| 162 | TGTGTTACCATATCC | A57160Hi | +T*+G*+T*G*T*T*A*C*C*A*T*A*T*C*C*A*+ | 79809 | 19 |
| | ATTT | | T*+T*+T | | |
| 163 | AACATACTTCATCTCT | A57161Hi | +A*+A*+C*A*T*A*C*T*T*C*A*T*C*T*C*T*+ | 80509 | 19 |
| | TCA | | T*+C*+A | | |
| 164 | GTCATAAGTTGTCAG | A57162Hi | +G*+T*+C*A*T*A*A*G*T*T*G*T*C*A*G*+T* | 80622 | 18 |
| | TAT | | +A*+T | | |
| 165 | TCCAAAGGTTTGTCA | A57163Hi | +T*+C*+C*A*A*A*G*G*T*T*T*G*T*C*A*T*+ | 80632 | 19 |
| | TAAG | | A*+A*+G | | |
| 166 | TATTTCTTAAGAATG | A57164Hi | +T*+A*+T*T*T*C*T*T*A*A*G*A*A*T*G*C*+ | 82190 | 19 |
| | CACC | | A*+C*+C | | |
| 167 | TCCTGATGATATGAC | A57165Hi | +T*+C*+C*T*G*A*T*G*A*T*A*T*G*A*C*C*+ | 82232 | 19 |
| | CAAA | | A*+A*+A | | |
| 168 | GTGGTCTTCATGACT | A57166Hi | +G*+T*+G*G*T*C*T*T*C*A*T*G*A*C*T*A*+ | 86182 | 19 |
| | AAAT | | A*+A*+T | | |
| 169 | GACTAAGTGAAATTC | A57167Hi | +G*+A*+C*T*A*A*G*T*G*A*A*A*T*T*C*A*+ | 86462 | 19 |
| | ATCA | | T*+C*+A | | |
| 170 | ACAAATGGCTCATTA | A57168Hi | +A*+C*+A*A*A*T*G*G*C*T*C*A*T*T*A*G*+ | 86707 | 19 |
| | GGTT | | G*+T*+T | | |
| 171 | CTGTGACTTTGCATC | A57169Hi | +C*+T*+G*T*G*A*C*T*T*T*G*C*A*T*C*A*+ | 87122 | 19 |
| | AACT | | A*+C*+T | | |
| 172 | AATTCAGGTTGAAGC | A57170Hi | +A*+A*+T*T*C*A*G*G*T*T*G*A*A*G*C*A*+ | 87444 | 19 |
| | ACCT | | C*+C*+T | | |
| 173 | CAACCATAACCATTA | A57171Hi | +C*+A*+A*C*C*A*T*A*A*C*C*A*T*T*A*C*+ | 87994 | 19 |
| | CTTC | | T*+T*+C | | |
| 174 | AGACAACCATAACCA | A57172Hi | +A*+G*+A*C*A*A*C*C*A*T*A*A*C*C*A*T*+ | 87997 | 19 |
| | TTAC | | T*+A*+C | | |
| 175 | TGGCAAATTCAGTGC | A57173Hi | +T*+G*+G*C*A*A*A*T*T*C*A*G*T*G*C*T*+ | 88896 | 19 |
| | TAAC | | A*+A*+C | | |
| 176 | TCAAATGGTTGGCAA | A57174Hi | +T*+C*+A*A*A*T*G*G*T*T*G*G*C*A*A*A*+ | 88905 | 19 |
| | ATTC | | T*+T*+C | | |
| 177 | ACTCCTAGGTGATTT | A57175Hi | +A*+C*+T*C*C*T*A*G*G*T*G*A*T*T*T*C*+ | 89376 | 19 |
| | CTAA | | T*+A*+A | | |
| 178 | ACACAATTAGCACTG | A57176Hi | +A*+C*+A*C*A*A*T*T*A*G*C*A*C*T*G*A*+ | 89684 | 19 |
| | AAGT | | A*+G*+T | | |
| 179 | ACTTACTCTAGTACT | A57177Hi | +A*+C*+T*T*A*C*T*C*T*A*G*T*A*C*T*A*+ | 89742 | 19 |
| | ATCA | | T*+C*+A | | |
| 180 | AGGAACAGATCATAA | A57178Hi | +A*+G*+G*A*A*C*A*G*A*T*C*A*T*A*A*+A* | 89817 | 18 |
| | ACC | | +C*+C | | |
| 181 | TCTATGCTTTGACCA | A57179Hi | +T*+C*+T*A*T*G*C*T*T*T*G*A*C*C*A*T*+ | 90335 | 19 |
| | TAAC | | A*+A*+C | | |
| 182 | AGTCTTCTATGTGAA | A57180Hi | +A*+G*+T*C*T*T*C*T*A*T*G*T*G*A*A*A*+ | 90437 | 19 |
| | ATGT | | T*+G*+T | | |
| 183 | AGTGAACTCAAGACA | A57181Hi | +A*+G*+T*G*A*A*C*T*C*A*A*G*A*C*A*T*+ | 90482 | 19 |
| | TCTT | | C*+T*+T | | |
| 184 | TCTAGATAGAGGTTT | A57182Hi | +T*+C*+T*A*G*A*T*A*G*A*G*G*T*T*T*+C* | 90672 | 18 |
| | CAC | | +A*+C | | |
| 185 | CAGCTACCTACACAT | A57183Hi | +C*+A*+G*C*T*A*C*C*T*A*C*A*C*A*T*C*+ | 91810 | 19 |
| | CTTT | | T*+T*+T | | |
| 186 | TTTATTGCAGTCATA | A57184Hi | +T*+T*+T*A*T*T*G*C*A*G*T*C*A*T*A*G*+ | 92086 | 19 |
| | GTGA | | T*+G*+A | | |
| 187 | CTGCCTTAATGGTAC | A57185Hi | +C*+T*+G*C*C*T*T*A*A*T*G*G*T*A*C*C*+ | 92545 | 19 |
| | CTTT | | T*+T*+T | | |
| 188 | ACAGTTAAATGAGTC | A57186Hi | +A*+C*+A*G*T*T*A*A*A*T*G*A*G*T*C*T*+ | 92574 | 19 |
| | TTGT | | T*+G*+T | | |
| 189 | GTACTTTATAGCAAT | A57187Hi | +G*+T*+A*C*T*T*T*A*T*A*G*C*A*A*T*+T* | 93827 | 18 |
| | TCC | | +C*+C | | |
| 190 | TCTCAGAACTTGCTT | A57188Hi | +T*+C*+T*C*A*G*A*A*C*T*T*G*C*T*T*A*+ | 95512 | 19 |
| | AATA | | A*+T*+A | | |
| 191 | ACACTATGGTTATAC | A57189Hi | +A*+C*+A*C*T*A*T*G*G*T*T*A*T*A*C*+A* | 96006 | 18 |
| | AGC | | +G*+C | | |
| 192 | AGTGATTGTAATACA | A57190Hi | +A*+G*+T*G*A*T*T*G*T*A*A*T*A*C*A*G*+ | 96949 | 19 |
| | GATC | | A*+T*+C | | |
| 193 | TTGTCTAAACCTCAT | A57191Hi | +T*+T*+G*T*C*T*A*A*A*C*C*T*C*A*T*+A* | 97276 | 18 |
| | ACT | | +C*+T | | |
| 194 | CTTGTCTAAACCTCA | A57192Hi | +C*+T*+T*G*T*C*T*A*A*A*C*C*T*C*A*+T* | 97277 | 18 |
| | TAC | | +A*+C | | |
| 195 | ATCCTAGTCTACATG | A57193Hi | +A*+T*+C*C*T*A*G*T*C*T*A*C*A*T*G*A*+ | 98498 | 19 |
| | ATTA | | T*+T*+A | | |
| 196 | CATGATAATCTCATC | A57194Hi | +C*+A*+T*G*A*T*A*A*T*C*T*C*A*T*C*C*+ | 98522 | 19 |
| | CTCA | | T*+C*+A | | |
| 197 | ATAGCTAAACTTCTC | A57195Hi | +A*+T*+A*G*C*T*A*A*A*C*T*T*C*T*C*C*+ | 99316 | 19 |
| | CATT | | A*+T*+T | | |
| 198 | TATGTCCTGACTGAC | A57196Hi | +T*+A*+T*G*T*C*C*T*G*A*C*T*G*A*C*+A* | 99612 | 18 |
| | AGA | | +G*+A | | |
| 199 | ACTAATACATTAACTT | A57197Hi | +A*+C*+T*A*A*T*A*C*A*T*T*A*A*C*T*T*+ | 99754 | 19 |
| | CCT | | C*+C*+T | | |
| 200 | GTCCATTCATCTGAA | A57198Hi | +G*+T*+C*C*A*T*T*C*A*T*C*T*G*A*A*A*+ | 99941 | 19 |
| | ACAC | | C*+A*+C | | |
| 201 | CGTTTCCACATACAC | A57199Hi | +C*+G*+T*T*T*C*C*A*C*A*T*A*C*A*C*T*+ | 99968 | 19 |
| | TACT | | A*+C*+T | | |
| 202 | CGTTTCCACATACAC | A57200Hi | +C*+G*+T*T*T*C*C*A*C*A*T*A*C*A*C*+T* | 99969 | 18 |
| | TAC | | +A*+C | | |
| 203 | ACGTTTCCACATACA | A57201Hi | +A*+C*+G*T*T*T*C*C*A*C*A*T*A*C*A*C*+ | 99969 | 19 |
| | CTAC | | T*+A*+C | | |
| 204 | CTTTGTAAATGACGT | A57202Hi | +C*+T*+T*T*G*T*A*A*A*T*G*A*C*G*T*T*+ | 99980 | 19 |
| | TTCC | | T*+C*+C | | |
| 205 | AACTCCTATCTAGAC | A57203Hi | +A*+A*+C*T*C*C*T*A*T*C*T*A*G*A*C*+T* | 100415 | 18 |
| | TGC | | +G*+C | | |
| 206 | CCTTATCTTTGGCAA | A57204Hi | +C*+C*+T*T*A*T*C*T*T*T*G*G*C*A*A*C*+ | 100594 | 19 |
| | CATC | | A*+T*+C | | |
| 207 | TCACTGCATAATAGT | A57205Hi | +T*+C*+A*C*T*G*C*A*T*A*A*T*A*G*T*+T* | 100809 | 18 |
| | TAC | | +A*+C | | |
| 208 | TCACTTGATGGATAT | A57206Hi | +T*+C*+A*C*T*T*G*A*T*G*G*A*T*A*T*A*+ | 102072 | 19 |
| | AAGT | | A*+G*+T | | |
| 209 | ACAATATTTATGGTT | A57207Hi | +A*+C*+A*A*T*A*T*T*T*A*T*G*G*T*T*C*+ | 102111 | 19 |
| | CTCA | | T*+C*+A | | |
| 209 | ACAATATTTATGGTT | A575329Hi | +A*+C*A*A*T*A*T*T*T*A*T*G*G*T*T*C*+T | 102111 | 19 |
| | CTCA | | *+C*+A | | |
| 209 | ACAATATTTATGGTT | A575330Hi | +A*C*+A*A*T*A*T*T*T*A*T*G*G*T*T*C*+T | 102111 | 19 |
| | CTCA | | *+C*+A | | |
| 209 | ACAATATTTATGGTT | A575332Hi | +A*+C*+A*A*T*A*T*T*T*A*T*G*G*T*T*C*+ | 102111 | 19 |
| | CTCA | | T*C*+A | | |
| 209 | ACAATATTTATGGTT | A575335Hi | +A*+C*+A*+A*T*A*T*T*T*A*T*G*G*T*T*C* | 102111 | 19 |
| | CTCA | | +T*+C*+A | | |
| 209 | ACAATATTTATGGTT | A575336Hi | +A*+C*+A*+A*T*A*T*T*T*A*T*G*G*T*T*C* | 102111 | 19 |
| | CTCA | | T*+C*+A | | |
| 209 | ACAATATTTATGGTT | A575337Hi | +A*+C*+A*A*T*A*T*T*T*A*T*G*G*T*T*+C* | 102111 | 19 |
| | CTCA | | +T*+C*+A | | |
| 209 | ACAATATTTATGGTT | A575338Hi | +A*+C*A*A*T*A*T*T*T*A*T*G*G*T*T*+C*+ | 102111 | 19 |
| | CTCA | | T*+C*+A | | |
| 209 | ACAATATTTATGGTT | A575340Hi | +A*+C*+A*+A*T*A*T*T*T*A*T*G*G*T*T*C* | 102111 | 19 |
| | CTCA | | +T*C*+A | | |
| 209 | ACAATATTTATGGTT | A575341Hi | +A*C*+A*A*T*A*T*T*T*A*T*G*G*T*T*+C*+ | 102111 | 19 |
| | CTCA | | T*+C*+A | | |
| 210 | TACAATATTTATGGTT | A57208Hi | +T*+A*+C*A*A*T*A*T*T*T*A*T*G*G*T*T*+ | 102112 | 19 |
| | CTC | | C*+T*+C | | |
| 210 | TACAATATTTATGGTT | A575343Hi | +T*+A*C*A*A*T*A*T*T*T*A*T*G*G*T*T*+C | 102112 | 19 |
| | CTC | | *+T*+C | | |
| 210 | TACAATATTTATGGTT | A575344Hi | +T*A*+C*A*A*T*A*T*T*T*A*T*G*G*T*T*+C | 102112 | 19 |
| | CTC | | *+T*+C | | |
| 210 | TACAATATTTATGGTT | A575346Hi | +T*+A*+C*A*A*T*A*T*T*T*A*T*G*G*T*T*+ | 102112 | 19 |
| | CTC | | C*T*+C | | |
| 210 | TACAATATTTATGGTT | A575349Hi | +T*+A*+C*+A*A*T*A*T*T*T*A*T*G*G*T*T* | 102112 | 19 |
| | CTC | | +C*+T*+C | | |
| 210 | TACAATATTTATGGTT | A575350Hi | +T*+A*+C*+A*A*T*A*T*T*T*A*T*G*G*T*T* | 102112 | 19 |
| | CTC | | C*+T*+C | | |
| 210 | TACAATATTTATGGTT | A575351Hi | +T*+A*+C*A*A*T*A*T*T*T*A*T*G*G*T*+T* | 102112 | 19 |
| | CTC | | +C*+T*+C | | |
| 210 | TACAATATTTATGGTT | A575352Hi | +T*+A*C*A*A*T*A*T*T*T*A*T*G*G*T*+T*+ | 102112 | 19 |
| | CTC | | C*+T*+C | | |
| 210 | TACAATATTTATGGTT | A575354Hi | +T*+A*+C*+A*A*T*A*T*T*T*A*T*G*G*T*T* | 102112 | 19 |
| | CTC | | +C*T*+C | | |
| 210 | TACAATATTTATGGTT | A575355Hi | +T*A*+C*A*A*T*A*T*T*T*A*T*G*G*T*+T*+ | 102112 | 19 |
| | CTC | | C*+T*+C | | |
| 211 | TGTACAATATTTATG | A57209Hi | +T*+G*+T*A*C*A*A*T*A*T*T*T*A*T*G*G*+ | 102114 | 19 |
| | GTTC | | T*+T*+C | | |
| 212 | GTGCTCTAGACAAAC | A57210Hi | +G*+T*+G*C*T*C*T*A*G*A*C*A*A*A*+C*+C | 102304 | 17 |
| | CG | | *+G | | |
| 213 | ATTCAGCTCCATAGT | A57211Hi | +A*+T*+T*C*A*G*C*T*C*C*A*T*A*G*T*C*+ | 102569 | 19 |
| | CATA | | A*+T*+A | | |
| 214 | CTTCCATATAGCTGA | A57212Hi | +C*+T*+T*C*C*A*T*A*T*A*G*C*T*G*A*+T* | 102915 | 18 |
| | TGA | | +G*+A | | |
| 215 | AACTCAATCACAGCC | A57213Hi | +A*+A*+C*T*C*A*A*T*C*A*C*A*G*C*C*+T* | 102980 | 18 |
| | TAT | | +A*+T | | |
| 216 | TTATAGCCACCTCCT | A57214Hi | +T*+T*+A*T*A*G*C*C*A*C*C*T*C*C*T*+A* | 103051 | 18 |
| | AAG | | +A*+G | | |
| 217 | CTTCTGTGACTTCAT | A57215Hi | +C*+T*+T*C*T*G*T*G*A*C*T*T*C*A*T*C*+ | 103101 | 19 |
| | CTAT | | T*+A*+T | | |
| 218 | AATTATGTCATCCAC | A57216Hi | +A*+A*+T*T*A*T*G*T*C*A*T*C*C*A*C*T*+ | 103115 | 19 |
| | TTCT | | T*+C*+T | | |
| 219 | AACTAGATATAGGCT | A57217Hi | +A*+A*+C*T*A*G*A*T*A*T*A*G*G*C*T*+C* | 105711 | 18 |
| | CCA | | +C*+A | | |
| 220 | CACGTATCTCCTTCTT | A57218Hi | +C*+A*+C*G*T*A*T*C*T*C*C*T*T*C*T*+T* | 109904 | 18 |
| | TA | | +T*+A | | |
| 221 | CCTGATCCAACTTTC | A57219Hi | +C*+C*+T*G*A*T*C*C*A*A*C*T*T*T*C*+A* | 111091 | 18 |
| | ATG | | +T*+G | | |
| 222 | CTTTGCTTATTCGTG | A57220Hi | +C*+T*+T*T*G*C*T*T*A*T*T*C*G*T*G*+A* | 114218 | 18 |
| | ACT | | +C*+T | | |
| 223 | CCTAATAAAGTGCAC | A57221Hi | +C*+C*+T*A*A*T*A*A*A*G*T*G*C*A*C*+C* | 119686 | 18 |
| | CTG | | +T*+G | | |
| 224 | CAATGTATACCATGC | A57222Hi | +C*+A*+A*T*G*T*A*T*A*C*C*A*T*G*C*+A* | 120997 | 18 |
| | AGT | | +G*+T | | |
| 225 | TCTTGCCTAGTATAT | A57223Hi | +T*+C*+T*T*G*C*C*T*A*G*T*A*T*A*T*+G* | 125919 | 18 |
| | GTT | | +T*+T | | |
| 226 | CCAATACTAACTTAA | A57224Hi | +C*+C*+A*A*T*A*C*T*A*A*C*T*T*A*A*+A* | 130119 | 18 |
| | ACC | | +C*+C | | |
| 227 | TTCAACTCCGAAACT | A57225Hi | +T*+T*+C*A*A*C*T*C*C*G*A*A*A*C*T*+G* | 130196 | 18 |
| | GCC | | +C*+C | | |
| 228 | GTTGATTGTCTTCCA | A57226Hi | +G*+T*+T*G*A*T*T*G*T*C*T*T*C*C*A*+A* | 134758 | 18 |
| | ATT | | +T*+T | | |
| 229 | GAATACTTGCTGTCT | A57227Hi | +G*+A*+A*T*A*C*T*T*G*C*T*G*T*C*T*+A* | 134850 | 18 |
| | ATC | | +T*+C | | |
| 230 | AAGAATACTTGCTGT | A57228Hi | +A*+A*+G*A*A*T*A*C*T*T*G*C*T*G*T*C*+ | 134851 | 19 |
| | CTAT | | T*+A*+T | | |
| 231 | ACAATCCTCAGGTAA | A57229Hi | +A*+C*+A*A*T*C*C*T*C*A*G*G*T*A*A*+G* | 135164 | 18 |
| | GAG | | +A*+G | | |
| 232 | ACTGGATGCTCTTGT | A57230Hi | +A*+C*+T*G*G*A*T*G*C*T*C*T*T*G*T*T*+ | 135523 | 19 |
| | TAAT | | A*+A*+T | | |
| 233 | ATGGATGCTCTCTAG | A57231Hi | +A*+T*+G*G*A*T*G*C*T*C*T*C*T*A*G*+T* | 136662 | 18 |
| | TAA | | +A*+A | | |
| 234 | TTGAGAGCTTGATCT | A57232Hi | +T*+T*+G*A*G*A*G*C*T*T*G*A*T*C*T*T*+ | 136678 | 19 |
| | TTAT | | T*+A*+T | | |
| 235 | CATCTTTGAGAGCTT | A57233Hi | +C*+A*+T*C*T*T*T*G*A*G*A*G*C*T*T*G*+ | 136683 | 19 |
| | GATC | | A*+T*+C | | |
| 236 | CACAAACAGATTGGC | A57234Hi | +C*+A*+C*A*A*A*C*A*G*A*T*T*G*G*C*+T* | 137734 | 18 |
| | TAC | | +A*+C | | |
| 237 | TCTACGTGTAATACA | A57235Hi | +T*+C*+T*A*C*G*T*G*T*A*A*T*A*C*A*+A* | 138335 | 18 |
| | ACC | | +C*+C | | |
| 238 | AAAGCTAGCAGAGAC | A57236Hi | +A*+A*+A*G*C*T*A*G*C*A*G*A*G*A*C*T*+ | 138834 | 19 |
| | TCCA | | C*+C*+A | | |
| 239 | GGACATGTATCTTAA | A57237Hi | +G*+G*+A*C*A*T*G*T*A*T*C*T*T*A*A*T*+ | 140893 | 19 |
| | TAGT | | A*+G*+T | | |
| 240 | AATCTGTGATCCACT | A57238Hi | +A*+A*+T*C*T*G*T*G*A*T*C*C*A*C*T*C*+ | 141809 | 19 |
| | CCTT | | C*+T*+T | | |
| 241 | TTCCTGATTATACCAT | A57239Hi | +T*+T*+C*C*T*G*A*T*T*A*T*A*C*C*A*T*+ | 142174 | 19 |
| | ATG | | A*+T*+G | | |
| 242 | ACATCCTCCTCCACG | A57240Hi | +A*+C*+A*T*C*C*T*C*C*T*C*C*A*C*G*+T* | 5111 | 18 |
| | TAA | | +A*+A | | |
| 243 | GCCTATTCACAACTT | A57241Hi | +G*+C*+C*T*A*T*T*C*A*C*A*A*C*T*T*C*+ | 30286 | 19 |
| | CATC | | A*+T*+C | | |
| 244 | AATCTTGCCATTCCTT | A57242Hi | +A*+A*+T*C*T*T*G*C*C*A*T*T*C*C*T*T*+ | 32561 | 19 |
| | TCA | | T*+C*+A | | |
| 245 | CACAATCTTGCCATT | A57243Hi | +C*+A*+C*A*A*T*C*T*T*G*C*C*A*T*T*C*+ | 32564 | 19 |
| | CCTT | | C*+T*+T | | |
| 246 | CCTTCAACATGTTTC | A57244Hi | +C*+C*+T*T*C*A*A*C*A*T*G*T*T*T*C*C*+ | 32579 | 19 |
| | CACA | | A*+C*+A | | |
| 247 | CTTCCAAGCTTCCTC | A57245Hi | +C*+T*+T*C*C*A*A*G*C*T*T*C*C*T*C*A*+ | 48546 | 19 |
| | ATTT | | T*+T*+T | | |
| 248 | TTATTCAACCTCTCTG | A57246Hi | +T*+T*+A*T*T*C*A*A*C*C*T*C*T*C*T*G*+ | 93533 | 19 |
| | TCA | | T*+C*+A | | |
| 249 | AGTCCTCATCCATAT | A57247Hi | +A*+G*+T*C*C*T*C*A*T*C*C*A*T*A*T*C*+ | 96890 | 19 |
| | CTAT | | T*+A*+T | | |
| 250 | ATGCTCTTATCCACA | A57248Hi | +A*+T*+G*C*T*C*T*T*A*T*C*C*A*C*A*A*+ | 135068 | 19 |
| | ATCC | | T*+C*+C | | |
| 251 | TACATATTTCACAAA | A57249Hi | +T*+A*+C*A*T*A*T*T*T*C*A*C*A*A*A*C*+ | 137742 | 19 |
| | CAGA | | A*+G*+A | | |
| 252 | TTGGACCAGCGTTTC | A57250H | +T*+T*+G*G*A*C*C*A*G*C*G*T*T*T*C*T*+ | 228 | 19 |
| | TATC | | A*+T*+C | | |
| 253 | TTGGACCAGCGTTTC | A57251H | +T*+T*+G*G*A*C*C*A*G*C*G*T*T*T*C*+T* | 229 | 18 |
| | TAT | | +A*+T | | |
| 254 | GACTTCGTTTAATAC | A57252H | +G*+A*+C*T*T*C*G*T*T*T*A*A*T*A*C*+C* | 24847 | 18 |
| | CAG | | +A*+G | | |
| 255 | GGACTTCGTTTAATA | A57253H | +G*+G*+A*C*T*T*C*G*T*T*T*A*A*T*A*C*+ | 24847 | 19 |
| | CCAG | | C*+A*+G | | |
| 256 | ATGGACTTCGTTTAA | A57254H | +A*+T*+G*G*A*C*T*T*C*G*T*T*T*A*A*T*+ | 24849 | 19 |
| | TACC | | A*+C*+C | | |
| 257 | TCGAAGCGCCTCCAG | A57255H | +T*+C*+G*A*A*G*C*G*C*C*T*C*C*A*+G*+ | 34567 | 17 |
| | CT | | C*+T | | |
| 258 | TGTTGATCCTTTTGTT | A57256H | +T*+G*+T*T*G*A*T*C*C*T*T*T*T*G*T*T*+ | 53001 | 19 |
| | CCA | | C*+C*+A | | |
| 259 | CTCGCTGCGTCATAA | A57257H | +C*+T*+C*G*C*T*G*C*G*T*C*A*T*A*A*A*+ | 84139 | 19 |
| | AATG | | A*+T*+G | | |
| 260 | CTCGCTGCGTCATAA | A57258H | +C*+T*+C*G*C*T*G*C*G*T*C*A*T*A*A*+A* | 84140 | 18 |
| | AAT | | +A*+T | | |
| 261 | CTCGCTGCGTCATAA | A57259H | +C*+T*+C*G*C*T*G*C*G*T*C*A*T*A*+A*+A | 84141 | 17 |
| | AA | | *+A | | |
| 262 | CTCGCTGCGTCATAA | A57260H | +C*+T*+C*G*C*T*G*C*G*T*C*A*T*+A*+A*+ | 84142 | 16 |
| | A | | A | | |
| 263 | GCAGGGGCGCCGGC | A57261H | +G*+C*+A*G*G*G*G*C*G*C*C*G*G*C*C*A* | 56 | 19 |
| | CACAG | | +C*+A*+G | | |
| 264 | GCAGGGGCGCCGGC | A57262H | +G*+C*+A*G*G*G*G*C*G*C*C*G*G*C*C*+A | 57 | 18 |
| | CACA | | *+C*+A | | |
| 265 | GGCAGGGGCGCCGG | A57263H | +G*+G*+C*A*G*G*G*G*C*G*C*C*G*G*C*C* | 57 | 19 |
| | CCACA | | +A*+C*+A | | |
| 266 | CCGGCAGGGGCGCC | A57264H | +C*+C*+G*G*C*A*G*G*G*G*C*G*C*C*G*G* | 59 | 19 |
| | GGCCA | | +C*+C*+A | | |
| 267 | GAACCGGCAGGGGC | A57265H | +G*+A*+A*C*C*G*G*C*A*G*G*G*G*C*G*C* | 62 | 19 |
| | GCCGG | | +C*+G*+G | | |
| 268 | GAACCGGCAGGGGC | A57266H | +G*+A*+A*C*C*G*G*C*A*G*G*G*G*C*G*+C | 63 | 18 |
| | GCCG | | *+C*+G | | |
| 269 | GGAACCGGCAGGGG | A57267H | +G*+G*+A*A*C*C*G*G*C*A*G*G*G*G*C*G* | 63 | 19 |
| | CGCCG | | +C*+C*+G | | |
| 270 | GGAACCGGCAGGGG | A57268H | +G*+G*+A*A*C*C*G*G*C*A*G*G*G*G*C*+G | 64 | 18 |
| | CGCC | | *+C*+C | | |
| 271 | GGGAACCGGCAGGG | A57269H | +G*+G*+G*A*A*C*C*G*G*C*A*G*G*G*G*C* | 64 | 19 |
| | GCGCC | | +G*+C*+C | | |
| 272 | AGGGAACCGGCAGG | A57270H | +A*+G*+G*G*A*A*C*C*G*G*C*A*G*G*G*G* | 65 | 19 |
| | GGCGC | | +C*+G*+C | | |
| 273 | TCAGGGAACCGGCAG | A57271H | +T*+C*+A*G*G*G*A*A*C*C*G*G*C*A*G*G* | 67 | 19 |
| | GGGC | | +G*+G*+C | | |
| 274 | TCAGGGAACCGGCAG | A57272H | +T*+C*+A*G*G*G*A*A*C*C*G*G*C*A*G*+G | 68 | 18 |
| | GGG | | *+G*+G | | |
| 275 | CTCAGGGAACCGGCA | A57273H | +C*+T*+C*A*G*G*G*A*A*C*C*G*G*C*A*G* | 68 | 19 |
| | GGGG | | +G*+G*+G | | |
| 276 | GCTCAGGGAACCGGC | A57274H | +G*+C*+T*C*A*G*G*G*A*A*C*C*G*G*C*A* | 69 | 19 |
| | AGGG | | +G*+G*+G | | |
| 277 | TGCTCAGGGAACCGG | A57275H | +T*+G*+C*T*C*A*G*G*G*A*A*C*C*G*G*C* | 70 | 19 |
| | CAGG | | +A*+G*+G | | |
| 278 | TGCTCAGGGAACCGG | A57276H | +T*+G*+C*T*C*A*G*G*G*A*A*C*C*G*G*+C | 71 | 18 |
| | CAG | | *+A*+G | | |
| 279 | CTGCTCAGGGAACCG | A57277H | +C*+T*+G*C*T*C*A*G*G*G*A*A*C*C*G*G* | 71 | 19 |
| | GCAG | | +C*+A*+G | | |
| 280 | CTGCTCAGGGAACCG | A57278H | +C*+T*+G*C*T*C*A*G*G*G*A*A*C*C*G*+G | 72 | 18 |
| | GCA | | *+C*+A | | |
| 281 | GCTGCTCAGGGAACC | A57279H | +G*+C*+T*G*C*T*C*A*G*G*G*A*A*C*C*G* | 72 | 19 |
| | GGCA | | +G*+C*+A | | |
| 282 | CGCTGCTCAGGGAAC | A57280H | +C*+G*+C*T*G*C*T*C*A*G*G*G*A*A*C*C*+ | 73 | 19 |
| | CGGC | | G*+G*+C | | |
| 283 | CGCTGCTCAGGGAAC | A57281H | +C*+G*+C*T*G*C*T*C*A*G*G*G*A*A*C*+C* | 74 | 18 |
| | CGG | | +G*+G | | |
| 284 | CCGCTGCTCAGGGAA | A57282H | +C*+C*+G*C*T*G*C*T*C*A*G*G*G*A*A*C*+ | 74 | 19 |
| | CCGG | | C*+G*+G | | |
| 285 | CGCTGCTCAGGGAAC | A57283H | +C*+G*+C*T*G*C*T*C*A*G*G*G*A*A*+C*+ | 75 | 17 |
| | CG | | C*+G | | |
| 286 | CCGCTGCTCAGGGAA | A57284H | +C*+C*+G*C*T*G*C*T*C*A*G*G*G*A*A*+C* | 75 | 18 |
| | CCG | | +C*+G | | |
| 287 | TCCGCTGCTCAGGGA | A57285H | +T*+C*+C*G*C*T*G*C*T*C*A*G*G*G*A*A*+ | 75 | 19 |
| | ACCG | | C*+C*+G | | |
| 288 | GTCCGCTGCTCAGGG | A57286H | +G*+T*+C*C*G*C*T*G*C*T*C*A*G*G*G*A*+ | 76 | 19 |
| | AACC | | A*+C*+C | | |
| 289 | CGTCCGCTGCTCAGG | A57287H | +C*+G*+T*C*C*G*C*T*G*C*T*C*A*G*G*G*+ | 77 | 19 |
| | GAAC | | A*+A*+C | | |
| 290 | CGTCCGCTGCTCAGG | A57288H | +C*+G*+T*C*C*G*C*T*G*C*T*C*A*G*G*+G* | 78 | 18 |
| | GAA | | +A*+A | | |
| 291 | ACGTCCGCTGCTCAG | A57289H | +A*+C*+G*T*C*C*G*C*T*G*C*T*C*A*G*G*+ | 78 | 19 |
| | GGAA | | G*+A*+A | | |
| 292 | CGTCCGCTGCTCAGG | A57290H | +C*+G*+T*C*C*G*C*T*G*C*T*C*A*G*+G*+ | 79 | 17 |
| | GA | | G*+A | | |
| 293 | ACGTCCGCTGCTCAG | A57291H | +A*+C*+G*T*C*C*G*C*T*G*C*T*C*A*G*+G* | 79 | 18 |
| | GGA | | +G*+A | | |
| 294 | AACGTCCGCTGCTCA | A57292H | +A*+A*+C*G*T*C*C*G*C*T*G*C*T*C*A*G*+ | 79 | 19 |
| | GGGA | | G*+G*+A | | |
| 295 | ACGTCCGCTGCTCAG | A57293H | +A*+C*+G*T*C*C*G*C*T*G*C*T*C*A*+G*+ | 80 | 17 |
| | GG | | G*+G | | |
| 296 | AACGTCCGCTGCTCA | A57294H | +A*+A*+C*G*T*C*C*G*C*T*G*C*T*C*A*+G* | 80 | 18 |
| | GGG | | +G*+G | | |
| 297 | GAACGTCCGCTGCTC | A57295H | +G*+A*+A*C*G*T*C*C*G*C*T*G*C*T*C*A*+ | 80 | 19 |
| | AGGG | | G*+G*+G | | |
| 298 | GAACGTCCGCTGCTC | A57296H | +G*+A*+A*C*G*T*C*C*G*C*T*G*C*T*C*+A* | 81 | 18 |
| | AGG | | +G*+G | | |
| 299 | TTCCAACCCGCCGCC | A57297H | +T*+T*+C*C*A*A*C*C*C*G*C*C*G*C*C*C*+ | 105 | 19 |
| | CTCC | | T*+C*+C | | |
| 300 | TTCCAACCCGCCGCC | A57298H | +T*+T*+C*C*A*A*C*C*C*G*C*C*G*C*C*+C* | 106 | 18 |
| | CTC | | +T*+C | | |
| 301 | CTTCCAACCCGCCGC | A57299H | +C*+T*+T*C*C*A*A*C*C*C*G*C*C*G*C*C*+ | 106 | 19 |
| | CCTC | | C*+T*+C | | |
| 302 | CTTCCAACCCGCCGC | A57300H | +C*+T*+T*C*C*A*A*C*C*C*G*C*C*G*C*+C* | 107 | 18 |
| | CCT | | +C*+T | | |
| 303 | GCTTCCAACCCGCCG | A57301H | +G*+C*+T*T*C*C*A*A*C*C*C*G*C*C*G*C*+ | 107 | 19 |
| | CCCT | | C*+C*+T | | |
| 304 | GCTTCCAACCCGCCG | A57302H | +G*+C*+T*T*C*C*A*A*C*C*C*G*C*C*G*+C* | 108 | 18 |
| | CCC | | +C*+C | | |
| 305 | TGCTTCCAACCCGCC | A57303H | +T*+G*+C*T*T*C*C*A*A*C*C*C*G*C*C*G*+ | 108 | 19 |
| | GCCC | | C*+C*+C | | |
| 306 | CCTGCTTCCAACCCG | A57304H | +C*+C*+T*G*C*T*T*C*C*A*A*C*C*C*G*C*+ | 110 | 19 |
| | CCGC | | C*+G*+C | | |
| 307 | CCTGCTTCCAACCCG | A57305H | +C*+C*+T*G*C*T*T*C*C*A*A*C*C*C*G*+C* | 111 | 18 |
| | CCG | | +C*+G | | |
| 308 | CTGCCACTAGCCATG | A57306H | +C*+T*+G*C*C*A*C*T*A*G*C*C*A*T*G*G*+ | 131 | 19 |
| | GTGG | | T*+G*+G | | |
| 309 | AGCTGCCACTAGCCA | A57307H | +A*+G*+C*T*G*C*C*A*C*T*A*G*C*C*A*T*+ | 133 | 19 |
| | TGGT | | G*+G*+T | | |
| 310 | AGCTGCCACTAGCCA | A57308H | +A*+G*+C*T*G*C*C*A*C*T*A*G*C*C*A*+T* | 134 | 18 |
| | TGG | | +G*+G | | |
| 311 | CAGCTGCCACTAGCC | A57309H | +C*+A*+G*C*T*G*C*C*A*C*T*A*G*C*C*A*+ | 134 | 19 |
| | ATGG | | T*+G*+G | | |
| 312 | GACAGCTGCCACTAG | A57310H | +G*+A*+C*A*G*C*T*G*C*C*A*C*T*A*G*C*+ | 136 | 19 |
| | CCAT | | C*+A*+T | | |
| 313 | CTGACAGCTGCCACT | A57311H | +C*+T*+G*A*C*A*G*C*T*G*C*C*A*C*T*A*+ | 138 | 19 |
| | AGCC | | G*+C*+C | | |
| 314 | CCCTGACAGCTGCCA | A57312H | +C*+C*+C*T*G*A*C*A*G*C*T*G*C*C*A*C*+ | 140 | 19 |
| | CTAG | | T*+A*+G | | |
| 315 | ACCCCTGACAGCTGC | A57313H | +A*+C*+C*C*C*T*G*A*C*A*G*C*T*G*C*C*+ | 142 | 19 |
| | CACT | | A*+C*+T | | |
| 316 | TCAGAGTTTCCTCGT | A57314H | +T*+C*+A*G*A*G*T*T*T*C*C*T*C*G*T*C*+ | 166 | 19 |
| | CCTC | | C*+T*+C | | |
| 317 | TCAGAGTTTCCTCGT | A57315H | +T*+C*+A*G*A*G*T*T*T*C*C*T*C*G*T*+C* | 167 | 18 |
| | CCT | | +C*+T | | |
| 318 | TTCAGAGTTTCCTCG | A57316H | +T*+T*+C*A*G*A*G*T*T*T*C*C*T*C*G*T*+ | 167 | 19 |
| | TCCT | | C*+C*+T | | |
| 319 | TCAGAGTTTCCTCGT | A57317H | +T*+C*+A*G*A*G*T*T*T*C*C*T*C*G*+T*+C | 168 | 17 |
| | CC | | *+C | | |
| 320 | TTCAGAGTTTCCTCG | A57318H | +T*+T*+C*A*G*A*G*T*T*T*C*C*T*C*G*+T* | 168 | 18 |
| | TCC | | +C*+C | | |
| 321 | CTTCAGAGTTTCCTC | A57319H | +C*+T*+T*C*A*G*A*G*T*T*T*C*C*T*C*G*+ | 168 | 19 |
| | GTCC | | T*+C*+C | | |
| 322 | TCTTCAGAGTTTCCT | A57320H | +T*+C*+T*T*C*A*G*A*G*T*T*T*C*C*T*C*+ | 169 | 19 |
| | CGTC | | G*+T*+C | | |
| 323 | TCTTCAGAGTTTCCT | A57321H | +T*+C*+T*T*C*A*G*A*G*T*T*T*C*C*T*+C* | 170 | 18 |
| | CGT | | +G*+T | | |
| 324 | TTCTTCAGAGTTTCCT | A57322H | +T*+T*+C*T*T*C*A*G*A*G*T*T*T*C*C*T*+ | 170 | 19 |
| | CGT | | C*+G*+T | | |
| 325 | TTCTTCAGAGTTTCCT | A57323H | +T*+T*+C*T*T*C*A*G*A*G*T*T*T*C*C*+T* | 171 | 18 |
| | CG | | +C*+G | | |
| 326 | CTTCTTCAGAGTTTC | A57324H | +C*+T*+T*C*T*T*C*A*G*A*G*T*T*T*C*C*+ | 171 | 19 |
| | CTCG | | T*+C*+G | | |
| 327 | GACTATCAACTTCTT | A57325H | +G*+A*+C*T*A*T*C*A*A*C*T*T*C*T*T*C*+ | 180 | 19 |
| | CAGA | | A*+G*+A | | |
| 328 | AGCCTGACTATCAAC | A57326H | +A*+G*+C*C*T*G*A*C*T*A*T*C*A*A*C*T*+ | 185 | 19 |
| | TTCT | | T*+C*+T | | |
| 329 | AGCCTGACTATCAAC | A57327H | +A*+G*+C*C*T*G*A*C*T*A*T*C*A*A*C*+T* | 186 | 18 |
| | TTC | | +T*+C | | |
| 330 | CAGCCTGACTATCAA | A57328H | +C*+A*+G*C*C*T*G*A*C*T*A*T*C*A*A*C*+ | 186 | 19 |
| | CTTC | | T*+T*+C | | |
| 331 | CAGCCTGACTATCAA | A57329H | +C*+A*+G*C*C*T*G*A*C*T*A*T*C*A*A*+C* | 187 | 18 |
| | CTT | | +T*+T | | |
| 332 | TCAGCCTGACTATCA | A57330H | +T*+C*+A*G*C*C*T*G*A*C*T*A*T*C*A*A*+ | 187 | 19 |
| | ACTT | | C*+T*+T | | |
| 333 | TCAGCCTGACTATCA | A57331H | +T*+C*+A*G*C*C*T*G*A*C*T*A*T*C*A*+A* | 188 | 18 |
| | ACT | | +C*+T | | |
| 334 | TTCAGCCTGACTATC | A57332H | +T*+T*+C*A*G*C*C*T*G*A*C*T*A*T*C*A*+ | 188 | 19 |
| | AACT | | A*+C*+T | | |
| 335 | TCAGCCTGACTATCA | A57333H | +T*+C*+A*G*C*C*T*G*A*C*T*A*T*C*+A*+A | 189 | 17 |
| | AC | | *+C | | |
| 336 | TTCAGCCTGACTATC | A57334H | +T*+T*+C*A*G*C*C*T*G*A*C*T*A*T*C*+A* | 189 | 18 |
| | AAC | | +A*+C | | |
| 337 | GTTCAGCCTGACTAT | A57335H | +G*+T*+T*C*A*G*C*C*T*G*A*C*T*A*T*C*+ | 189 | 19 |
| | CAAC | | A*+A*+C | | |
| 338 | GTTCAGCCTGACTAT | A57336H | +G*+T*+T*C*A*G*C*C*T*G*A*C*T*A*T*+C* | 190 | 18 |
| | CAA | | +A*+A | | |
| 339 | TGTTCAGCCTGACTA | A57337H | +T*+G*+T*T*C*A*G*C*C*T*G*A*C*T*A*T*+ | 190 | 19 |
| | TCAA | | C*+A*+A | | |
| 340 | TGTTCAGCCTGACTA | A57338H | +T*+G*+T*T*C*A*G*C*C*T*G*A*C*T*A*+T* | 191 | 18 |
| | TCA | | +C*+A | | |
| 341 | TTGTTCAGCCTGACT | A57339H | +T*+T*+G*T*T*C*A*G*C*C*T*G*A*C*T*A*+ | 191 | 19 |
| | ATCA | | T*+C*+A | | |
| 342 | CATTGTTCAGCCTGA | A57340H | +C*+A*+T*T*G*T*T*C*A*G*C*C*T*G*A*C*+ | 193 | 19 |
| | CTAT | | T*+A*+T | | |
| 343 | ACATTGTTCAGCCTG | A57341H | +A*+C*+A*T*T*G*T*T*C*A*G*C*C*T*G*A*+ | 194 | 19 |
| | ACTA | | C*+T*+A | | |
| 344 | GACATTGTTCAGCCT | A57342H | +G*+A*+C*A*T*T*G*T*T*C*A*G*C*C*T*G*+ | 195 | 19 |
| | GACT | | A*+C*+T | | |
| 345 | CCTGGACATTGTTCA | A57343H | +C*+C*+T*G*G*A*C*A*T*T*G*T*T*C*A*G*+ | 199 | 19 |
| | GCCT | | C*+C*+T | | |
| 346 | TCCTGGACATTGTTC | A57344H | +T*+C*+C*T*G*G*A*C*A*T*T*G*T*T*C*A*+ | 200 | 19 |
| | AGCC | | G*+C*+C | | |
| 347 | GACCAGCGTTTCTAT | A57345H | +G*+A*+C*C*A*G*C*G*T*T*T*C*T*A*T*C*+ | 225 | 19 |
| | CTGT | | T*+G*+T | | |
| 348 | GACCAGCGTTTCTAT | A57346H | +G*+A*+C*C*A*G*C*G*T*T*T*C*T*A*T*+C* | 226 | 18 |
| | CTG | | +T*+G | | |
| 349 | GGACCAGCGTTTCTA | A57347H | +G*+G*+A*C*C*A*G*C*G*T*T*T*C*T*A*T*+ | 226 | 19 |
| | TCTG | | C*+T*+G | | |
| 350 | TGGACCAGCGTTTCT | A57348H | +T*+G*+G*A*C*C*A*G*C*G*T*T*T*C*T*A*+ | 227 | 19 |
| | ATCT | | T*+C*+T | | |
| 351 | TGGACCAGCGTTTCT | A57349H | +T*+G*+G*A*C*C*A*G*C*G*T*T*T*C*T*+A* | 228 | 18 |
| | ATC | | +T*+C | | |
| 352 | TGGACCAGCGTTTCT | A57350H | +T*+G*+G*A*C*C*A*G*C*G*T*T*T*C*+T*+A | 229 | 17 |
| | AT | | *+T | | |
| 353 | TTTGGACCAGCGTTT | A57351H | +T*+T*+T*G*G*A*C*C*A*G*C*G*T*T*T*C*+ | 229 | 19 |
| | CTAT | | T*+A*+T | | |
| 354 | TTGGACCAGCGTTTC | A57352H | +T*+T*+G*G*A*C*C*A*G*C*G*T*T*T*+C*+T | 230 | 17 |
| | TA | | *+A | | |
| 355 | TTTGGACCAGCGTTT | A57353H | +T*+T*+T*G*G*A*C*C*A*G*C*G*T*T*T*+C* | 230 | 18 |
| | CTA | | +T*+A | | |
| 356 | ATTTGGACCAGCGTT | A57354H | +A*+T*+T*T*G*G*A*C*C*A*G*C*G*T*T*T*+ | 230 | 19 |
| | TCTA | | C*+T*+A | | |
| 357 | GATTTGGACCAGCGT | A57355H | +G*+A*+T*T*T*G*G*A*C*C*A*G*C*G*T*T*+ | 231 | 19 |
| | TTCT | | T*+C*+T | | |
| 358 | GATTTGGACCAGCGT | A57356H | +G*+A*+T*T*T*G*G*A*C*C*A*G*C*G*T*+T* | 232 | 18 |
| | TTC | | +T*+C | | |
| 359 | GGATTTGGACCAGCG | A57357H | +G*+G*+A*T*T*T*G*G*A*C*C*A*G*C*G*T*+ | 232 | 19 |
| | TTTC | | T*+T*+C | | |
| 360 | GATTTGGACCAGCGT | A57358H | +G*+A*+T*T*T*G*G*A*C*C*A*G*C*G*+T*+T | 233 | 17 |
| | TT | | *+T | | |
| 361 | GGATTTGGACCAGCG | A57359H | +G*+G*+A*T*T*T*G*G*A*C*C*A*G*C*G*+T* | 233 | 18 |
| | TTT | | +T*+T | | |
| 362 | AGGATTTGGACCAGC | A57360H | +A*+G*+G*A*T*T*T*G*G*A*C*C*A*G*C*G*+ | 233 | 19 |
| | GTTT | | T*+T*+T | | |
| 363 | AGGATTTGGACCAGC | A57361H | +A*+G*+G*A*T*T*T*G*G*A*C*C*A*G*C*+G* | 234 | 18 |
| | GTT | | +T*+T | | |
| 364 | CAGGATTTGGACCAG | A57362H | +C*+A*+G*G*A*T*T*T*G*G*A*C*C*A*G*C*+ | 234 | 19 |
| | CGTT | | G*+T*+T | | |
| 365 | CCAGGATTTGGACCA | A57363H | +C*+C*+A*G*G*A*T*T*T*G*G*A*C*C*A*G*+ | 235 | 19 |
| | GCGT | | C*+G*+T | | |
| 366 | TCCTCCAGGATTTGG | A57364H | +T*+C*+C*T*C*C*A*G*G*A*T*T*T*G*G*A*+ | 239 | 19 |
| | ACCA | | C*+C*+A | | |
| 367 | TCCTCCAGGATTTGG | A57365H | +T*+C*+C*T*C*C*A*G*G*A*T*T*T*G*G*+A* | 240 | 18 |
| | ACC | | +C*+C | | |
| 368 | ATCCTCCAGGATTTG | A57366H | +A*+T*+C*C*T*C*C*A*G*G*A*T*T*T*G*G*+ | 240 | 19 |
| | GACC | | A*+C*+C | | |
| 369 | ATCCTCCAGGATTTG | A57367H | +A*+T*+C*C*T*C*C*A*G*G*A*T*T*T*G*+G* | 241 | 18 |
| | GAC | | +A*+C | | |
| 370 | GATCCTCCAGGATTT | A57368H | +G*+A*+T*C*C*T*C*C*A*G*G*A*T*T*T*G*+ | 241 | 19 |
| | GGAC | | G*+A*+C | | |
| 371 | ACCAGCAGATCCTCC | A57369H | +A*+C*+C*A*G*C*A*G*A*T*C*C*T*C*C*A*+ | 248 | 19 |
| | AGGA | | G*+G*+A | | |
| 372 | AACACCAGCAGATCC | A57370H | +A*+A*+C*A*C*C*A*G*C*A*G*A*T*C*C*T*+ | 251 | 19 |
| | TCCA | | C*+C*+A | | |
| 373 | TGAACACCAGCAGAT | A57371H | +T*+G*+A*A*C*A*C*C*A*G*C*A*G*A*T*C*+ | 253 | 19 |
| | CCTC | | C*+T*+C | | |
| 374 | GTGAACACCAGCAGA | A57372H | +G*+T*+G*A*A*C*A*C*C*A*G*C*A*G*A*T*+ | 254 | 19 |
| | TCCT | | C*+C*+T | | |
| 375 | ACGTGAACACCAGCA | A57373H | +A*+C*+G*T*G*A*A*C*A*C*C*A*G*C*A*G*+ | 256 | 19 |
| | GATC | | A*+T*+C | | |
| 376 | TACGTGAACACCAGC | A57374H | +T*+A*+C*G*T*G*A*A*C*A*C*C*A*G*C*A*+ | 257 | 19 |
| | AGAT | | G*+A*+T | | |
| 377 | GTACGTGAACACCAG | A57375H | +G*+T*+A*C*G*T*G*A*A*C*A*C*C*A*G*C*+ | 258 | 19 |
| | CAGA | | A*+G*+A | | |
| 378 | AGTACGTGAACACCA | A57376H | +A*+G*+T*A*C*G*T*G*A*A*C*A*C*C*A*G*+ | 259 | 19 |
| | GCAG | | C*+A*+G | | |
| 379 | GAGTACGTGAACACC | A57377H | +G*+A*+G*T*A*C*G*T*G*A*A*C*A*C*C*A*+ | 260 | 19 |
| | AGCA | | G*+C*+A | | |
| 380 | GGAGTACGTGAACAC | A57378H | +G*+G*+A*G*T*A*C*G*T*G*A*A*C*A*C*C*+ | 261 | 19 |
| | CAGC | | A*+G*+C | | |
| 381 | CGGAGTACGTGAACA | A57379H | +C*+G*+G*A*G*T*A*C*G*T*G*A*A*C*A*C*+ | 262 | 19 |
| | CCAG | | C*+A*+G | | |
| 382 | CGGAGTACGTGAACA | A57380H | +C*+G*+G*A*G*T*A*C*G*T*G*A*A*C*A*+C* | 263 | 18 |
| | CCA | | +C*+A | | |
| 383 | TCGGAGTACGTGAAC | A57381H | +T*+C*+G*G*A*G*T*A*C*G*T*G*A*A*C*A*+ | 263 | 19 |
| | ACCA | | C*+C*+A | | |
| 384 | CGGAGTACGTGAACA | A57382H | +C*+G*+G*A*G*T*A*C*G*T*G*A*A*C*+A*+ | 264 | 17 |
| | CC | | C*+C | | |
| 385 | TCGGAGTACGTGAAC | A57383H | +T*+C*+G*G*A*G*T*A*C*G*T*G*A*A*C*+A* | 264 | 18 |
| | ACC | | +C*+C | | |
| 386 | CTCGGAGTACGTGAA | A57384H | +C*+T*+C*G*G*A*G*T*A*C*G*T*G*A*A*C*+ | 264 | 19 |
| | CACC | | A*+C*+C | | |
| 387 | TCGGAGTACGTGAAC | A57385H | +T*+C*+G*G*A*G*T*A*C*G*T*G*A*A*+C*+ | 265 | 17 |
| | AC | | A*+C | | |
| 388 | CTCGGAGTACGTGAA | A57386H | +C*+T*+C*G*G*A*G*T*A*C*G*T*G*A*A*+C* | 265 | 18 |
| | CAC | | +A*+C | | |
| 389 | GCTCGGAGTACGTGA | A57387H | +G*+C*+T*C*G*G*A*G*T*A*C*G*T*G*A*A*+ | 265 | 19 |
| | ACAC | | C*+A*+C | | |
| 390 | TCGGAGTACGTGAAC | A57388H | +T*+C*+G*G*A*G*T*A*C*G*T*G*A*+A*+C*+ | 266 | 16 |
| | A | | A | | |
| 391 | CTCGGAGTACGTGAA | A57389H | +C*+T*+C*G*G*A*G*T*A*C*G*T*G*A*+A*+ | 266 | 17 |
| | CA | | C*+A | | |
| 392 | GCTCGGAGTACGTGA | A57390H | +G*+C*+T*C*G*G*A*G*T*A*C*G*T*G*A*+A* | 266 | 18 |
| | ACA | | +C*+A | | |
| 393 | GCTCGGAGTACGTGA | A57391H | +G*+C*+T*C*G*G*A*G*T*A*C*G*T*G*+A*+ | 267 | 17 |
| | AC | | A*+C | | |
| 394 | AAAGGTGAAGACCTT | A57392Hi | +A*+A*+A*G*G*T*G*A*A*G*A*C*C*T*T*C*+ | 451 | 19 |
| | CCTG | | C*+T*+G | | |
| 395 | GTCACAATCCACAGC | A57393Hi | +G*+T*+C*A*C*A*A*T*C*C*A*C*A*G*C*+A* | 508 | 18 |
| | ACC | | +C*+C | | |
| 396 | AGAAGCAAAGTCACA | A57394Hi | +A*+G*+A*A*G*C*A*A*A*G*T*C*A*C*A*A*+ | 516 | 19 |
| | ATCC | | T*+C*+C | | |
| 397 | GCCTTGAAATAACTT | A57395H | +G*+C*+C*T*T*G*A*A*A*T*A*A*C*T*T*G*+ | 560 | 19 |
| | GGAG | | G*+A*+G | | |
| 398 | TTGCCTTGAAATAAC | A57396H | +T*+T*+G*C*C*T*T*G*A*A*A*T*A*A*C*T*+ | 562 | 19 |
| | TTGG | | T*+G*+G | | |
| 399 | GCACATGGATATTTT | A57397H | +G*+C*+A*C*A*T*G*G*A*T*A*T*T*T*T*T*+ | 576 | 19 |
| | TGCC | | G*+C*+C | | |
| 400 | GGCACATGGATATTT | A57398H | +G*+G*+C*A*C*A*T*G*G*A*T*A*T*T*T*T*+ | 577 | 19 |
| | TTGC | | T*+G*+C | | |
| 401 | GATCAACAGAGGCAC | A57399H | +G*+A*+T*C*A*A*C*A*G*A*G*G*C*A*C*A*+ | 587 | 19 |
| | ATGG | | T*+G*+G | | |
| 402 | GATCAACAGAGGCAC | A57400H | +G*+A*+T*C*A*A*C*A*G*A*G*G*C*A*C*+A* | 588 | 18 |
| | ATG | | +T*+G | | |
| 403 | CGATCAACAGAGGCA | A57401H | +C*+G*+A*T*C*A*A*C*A*G*A*G*G*C*A*C*+ | 588 | 19 |
| | CATG | | A*+T*+G | | |
| 404 | CGATCAACAGAGGCA | A57402H | +C*+G*+A*T*C*A*A*C*A*G*A*G*G*C*A*+C* | 589 | 18 |
| | CAT | | +A*+T | | |
| 405 | ACGATCAACAGAGGC | A57403H | +A*+C*+G*A*T*C*A*A*C*A*G*A*G*G*C*A*+ | 589 | 19 |
| | ACAT | | C*+A*+T | | |
| 406 | GACGATCAACAGAGG | A57404H | +G*+A*+C*G*A*T*C*A*A*C*A*G*A*G*G*C* | 590 | 19 |
| | CACA | | +A*+C*+A | | |
| 407 | GACGATCAACAGAGG | A57405H | +G*+A*+C*G*A*T*C*A*A*C*A*G*A*G*G*+C | 591 | 18 |
| | CAC | | *+A*+C | | |
| 408 | AGACGATCAACAGAG | A57406H | +A*+G*+A*C*G*A*T*C*A*A*C*A*G*A*G*G* | 591 | 19 |
| | GCAC | | +C*+A*+C | | |
| 409 | AGACGATCAACAGAG | A57407H | +A*+G*+A*C*G*A*T*C*A*A*C*A*G*A*G*+G | 592 | 18 |
| | GCA | | *+C*+A | | |
| 410 | AAGACGATCAACAGA | A57408H | +A*+A*+G*A*C*G*A*T*C*A*A*C*A*G*A*G*+ | 592 | 19 |
| | GGCA | | G*+C*+A | | |
| 411 | CAAGACGATCAACAG | A57409H | +C*+A*+A*G*A*C*G*A*T*C*A*A*C*A*G*A*+ | 593 | 19 |
| | AGGC | | G*+G*+C | | |
| 412 | CAAGACGATCAACAG | A57410H | +C*+A*+A*G*A*C*G*A*T*C*A*A*C*A*G*+A* | 594 | 18 |
| | AGG | | +G*+G | | |
| 413 | CCAAGACGATCAACA | A57411H | +C*+C*+A*A*G*A*C*G*A*T*C*A*A*C*A*G*+ | 594 | 19 |
| | GAGG | | A*+G*+G | | |
| 414 | CCAAGACGATCAACA | A57412H | +C*+C*+A*A*G*A*C*G*A*T*C*A*A*C*A*+G* | 595 | 18 |
| | GAG | | +A*+G | | |
| 415 | TCCAAGACGATCAAC | A57413H | +T*+C*+C*A*A*G*A*C*G*A*T*C*A*A*C*A*+ | 595 | 19 |
| | AGAG | | G*+A*+G | | |
| 416 | GTCCAAGACGATCAA | A57414H | +G*+T*+C*C*A*A*G*A*C*G*A*T*C*A*A*C*+ | 596 | 19 |
| | CAGA | | A*+G*+A | | |
| 417 | GTCCAAGACGATCAA | A57415H | +G*+T*+C*C*A*A*G*A*C*G*A*T*C*A*A*+C* | 597 | 18 |
| | CAG | | +A*+G | | |
| 418 | AGTCCAAGACGATCA | A57416H | +A*+G*+T*C*C*A*A*G*A*C*G*A*T*C*A*A*+ | 597 | 19 |
| | ACAG | | C*+A*+G | | |
| 419 | GTCCAAGACGATCAA | A57417H | +G*+T*+C*C*A*A*G*A*C*G*A*T*C*A*+A*+C | 598 | 17 |
| | CA | | *+A | | |
| 420 | AGTCCAAGACGATCA | A57418H | +A*+G*+T*C*C*A*A*G*A*C*G*A*T*C*A*+A* | 598 | 18 |
| | ACA | | +C*+A | | |
| 421 | GAGTCCAAGACGATC | A57419H | +G*+A*+G*T*C*C*A*A*G*A*C*G*A*T*C*A*+ | 598 | 19 |
| | AACA | | A*+C*+A | | |
| 422 | GAGTCCAAGACGATC | A57420H | +G*+A*+G*T*C*C*A*A*G*A*C*G*A*T*C*+A* | 599 | 18 |
| | AAC | | +A*+C | | |
| 423 | TCGCGACTCTCATAT | A57421H | +T*+C*+G*C*G*A*C*T*C*T*C*A*T*+A*+T*+ | 618 | 16 |
| | A | | A | | |
| 424 | CTCGCGACTCTCATA | A57422H | +C*+T*+C*G*C*G*A*C*T*C*T*C*A*T*+A*+T | 618 | 17 |
| | TA | | *+A | | |
| 425 | ACTCGCGACTCTCAT | A57423H | +A*+C*+T*C*G*C*G*A*C*T*C*T*C*A*T*+A* | 618 | 18 |
| | ATA | | +T*+A | | |
| 426 | CACTCGCGACTCTCA | A57424H | +C*+A*+C*T*C*G*C*G*A*C*T*C*T*C*A*T*+ | 618 | 19 |
| | TATA | | A*+T*+A | | |
| 427 | ACTCGCGACTCTCAT | A57425H | +A*+C*+T*C*G*C*G*A*C*T*C*T*C*A*+T*+A | 619 | 17 |
| | AT | | *+T | | |
| 428 | CACTCGCGACTCTCA | A57426H | +C*+A*+C*T*C*G*C*G*A*C*T*C*T*C*A*+T* | 619 | 18 |
| | TAT | | +A*+T | | |
| 429 | ACACTCGCGACTCTC | A57427H | +A*+C*+A*C*T*C*G*C*G*A*C*T*C*T*C*A*+ | 619 | 19 |
| | ATAT | | T*+A*+T | | |
| 430 | ACACTCGCGACTCTC | A57428H | +A*+C*+A*C*T*C*G*C*G*A*C*T*C*T*C*+A* | 620 | 18 |
| | ATA | | +T*+A | | |
| 431 | CACACTCGCGACTCT | A57429H | +C*+A*+C*A*C*T*C*G*C*G*A*C*T*C*T*C*+ | 620 | 19 |
| | CATA | | A*+T*+A | | |
| 432 | ACACTCGCGACTCTC | A57430H | +A*+C*+A*C*T*C*G*C*G*A*C*T*C*T*+C*+A | 621 | 17 |
| | AT | | *+T | | |
| 433 | CACACTCGCGACTCT | A57431H | +C*+A*+C*A*C*T*C*G*C*G*A*C*T*C*T*+C* | 621 | 18 |
| | CAT | | +A*+T | | |
| 434 | GCACACTCGCGACTC | A57432H | +G*+C*+A*C*A*C*T*C*G*C*G*A*C*T*C*T*+ | 621 | 19 |
| | TCAT | | C*+A*+T | | |
| 435 | CACACTCGCGACTCT | A57433H | +C*+A*+C*A*C*T*C*G*C*G*A*C*T*C*+T*+C | 622 | 17 |
| | CA | | *+A | | |
| 436 | GCACACTCGCGACTC | A57434H | +G*+C*+A*C*A*C*T*C*G*C*G*A*C*T*C*+T* | 622 | 18 |
| | TCA | | +C*+A | | |
| 437 | TGCACACTCGCGACT | A57435H | +T*+G*+C*A*C*A*C*T*C*G*C*G*A*C*T*C*+ | 622 | 19 |
| | CTCA | | T*+C*+A | | |
| 438 | TGCACACTCGCGACT | A57436H | +T*+G*+C*A*C*A*C*T*C*G*C*G*A*C*T*+C* | 623 | 18 |
| | CTC | | +T*+C | | |
| 439 | CTGCACACTCGCGAC | A57437H | +C*+T*+G*C*A*C*A*C*T*C*G*C*G*A*C*T*+ | 623 | 19 |
| | TCTC | | C*+T*+C | | |
| 440 | TGCACACTCGCGACT | A57438H | +T*+G*+C*A*C*A*C*T*C*G*C*G*A*C*+T*+C | 624 | 17 |
| | CT | | *+T | | |
| 441 | CTGCACACTCGCGAC | A57439H | +C*+T*+G*C*A*C*A*C*T*C*G*C*G*A*C*+T* | 624 | 18 |
| | TCT | | +C*+T | | |
| 442 | GCTGCACACTCGCGA | A57440H | +G*+C*+T*G*C*A*C*A*C*T*C*G*C*G*A*C*+ | 624 | 19 |
| | CTCT | | T*+C*+T | | |
| 443 | CTGCACACTCGCGAC | A57441H | +C*+T*+G*C*A*C*A*C*T*C*G*C*G*A*+C*+T | 625 | 17 |
| | TC | | *+C | | |
| 444 | GCTGCACACTCGCGA | A57442H | +G*+C*+T*G*C*A*C*A*C*T*C*G*C*G*A*+C* | 625 | 18 |
| | CTC | | +T*+C | | |
| 445 | TGCTGCACACTCGCG | A57443H | +T*+G*+C*T*G*C*A*C*A*C*T*C*G*C*G*A*+ | 625 | 19 |
| | ACTC | | C*+T*+C | | |
| 446 | GCTGCACACTCGCGA | A57444H | +G*+C*+T*G*C*A*C*A*C*T*C*G*C*G*+A*+ | 626 | 17 |
| | CT | | C*+T | | |
| 447 | TGCTGCACACTCGCG | A57445H | +T*+G*+C*T*G*C*A*C*A*C*T*C*G*C*G*+A* | 626 | 18 |
| | ACT | | +C*+T | | |
| 448 | CTGCTGCACACTCGC | A57446H | +C*+T*+G*C*T*G*C*A*C*A*C*T*C*G*C*G*+ | 626 | 19 |
| | GACT | | A*+C*+T | | |
| 449 | TGCTGCACACTCGCG | A57447H | +T*+G*+C*T*G*C*A*C*A*C*T*C*G*C*+G*+A | 627 | 17 |
| | AC | | *+C | | |
| 450 | CTGCTGCACACTCGC | A57448H | +C*+T*+G*C*T*G*C*A*C*A*C*T*C*G*C*+G* | 627 | 18 |
| | GAC | | +A*+C | | |
| 451 | CTGCTGCACACTCGC | A57449H | +C*+T*+G*C*T*G*C*A*C*A*C*T*C*G*+C*+G | 628 | 17 |
| | GA | | *+A | | |
| 452 | AAACGTCTCCTTCCA | A57450Hi | +A*+A*+A*C*G*T*C*T*C*C*T*T*C*C*A*A*+ | 683 | 19 |
| | AACA | | A*+C*+A | | |
| 453 | AGGTTCCTCAAGCCA | A57451Hi | +A*+G*+G*T*T*C*C*T*C*A*A*G*C*C*A*A*+ | 746 | 19 |
| | AATT | | A*+T*+T | | |
| 454 | AGGTTCCTCAAGCCA | A57452Hi | +A*+G*+G*T*T*C*C*T*C*A*A*G*C*C*A*+A* | 747 | 18 |
| | AAT | | +A*+T | | |
| 455 | GAGGTTCCTCAAGCC | A57453Hi | +G*+A*+G*G*T*T*C*C*T*C*A*A*G*C*C*A*+ | 747 | 19 |
| | AAAT | | A*+A*+T | | |
| 456 | GAACTCTACAAGAGT | A57454Hi | +G*+A*+A*C*T*C*T*A*C*A*A*G*A*G*T*T*+ | 1074 | 19 |
| | TTGC | | T*+G*+C | | |
| 457 | AACTCTACAAGAGTT | A57455Hi | +A*+A*+C*T*C*T*A*C*A*A*G*A*G*T*T*+T* | 1074 | 18 |
| | TGC | | +G*+C | | |
| 458 | TGCTGAACTCTACAA | A57456Hi | +T*+G*+C*T*G*A*A*C*T*C*T*A*C*A*A*G*+ | 1078 | 19 |
| | GAGT | | A*+G*+T | | |
| 459 | TCATTCTTAATTACCA | A57457Hi | +T*+C*+A*T*T*C*T*T*A*A*T*T*A*C*C*A*+ | 1298 | 19 |
| | TTT | | T*+T*+T | | |
| 460 | GTAAATCATTCTTAAT | A57458Hi | +G*+T*+A*A*A*T*C*A*T*T*C*T*T*A*A*T*+ | 1303 | 19 |
| | TAC | | T*+A*+C | | |
| 461 | GACTGTAAATCATTC | A57459Hi | +G*+A*+C*T*G*T*A*A*A*T*C*A*T*T*C*T*+ | 1307 | 19 |
| | TTAA | | T*+A*+A | | |
| 462 | GTTAACATGCAGATG | A57460Hi | +G*+T*+T*A*A*C*A*T*G*C*A*G*A*T*G*A*+ | 1397 | 19 |
| | ACAA | | C*+A*+A | | |
| 463 | AGTTAACATGCAGAT | A57461Hi | +A*+G*+T*T*A*A*C*A*T*G*C*A*G*A*T*G*+ | 1398 | 19 |
| | GACA | | A*+C*+A | | |
| 464 | GAGTTAACATGCAGA | A57462Hi | +G*+A*+G*T*T*A*A*C*A*T*G*C*A*G*A*T*+ | 1399 | 19 |
| | TGAC | | G*+A*+C | | |
| 465 | AGTTAACATGCAGAT | A57463Hi | +A*+G*+T*T*A*A*C*A*T*G*C*A*G*A*T*+G* | 1399 | 18 |
| | GAC | | +A*+C | | |
| 466 | TGCATGAGTTAACAT | A57464Hi | +T*+G*+C*A*T*G*A*G*T*T*A*A*C*A*T*G*+ | 1404 | 19 |
| | GCAG | | C*+A*+G | | |
| 467 | GCTTTCTGACTGCAC | A57465Hi | +G*+C*+T*T*T*C*T*G*A*C*T*G*C*A*C*C*+ | 1452 | 19 |
| | CTCC | | T*+C*+C | | |
| 468 | GCTTTCTGACTGCAC | A57466Hi | +G*+C*+T*T*T*C*T*G*A*C*T*G*C*A*C*+C* | 1453 | 18 |
| | CTC | | +T*+C | | |
| 469 | TGGCTTTCTGACTGC | A57467Hi | +T*+G*+G*C*T*T*T*C*T*G*A*C*T*G*C*A*+ | 1454 | 19 |
| | ACCT | | C*+C*+T | | |
| 470 | TGGCTTTCTGACTGC | A57468Hi | +T*+G*+G*C*T*T*T*C*T*G*A*C*T*G*C*+A* | 1455 | 18 |
| | ACC | | +C*+C | | |
| 471 | TTGGCTTTCTGACTG | A57469Hi | +T*+T*+G*G*C*T*T*T*C*T*G*A*C*T*G*C*+ | 1455 | 19 |
| | CACC | | A*+C*+C | | |
| 472 | ATTGGCTTTCTGACT | A57470Hi | +A*+T*+T*G*G*C*T*T*T*C*T*G*A*C*T*G*+ | 1456 | 19 |
| | GCAC | | C*+A*+C | | |
| 473 | TTAAGAAACATTGGC | A57471Hi | +T*+T*+A*A*G*A*A*A*C*A*T*T*G*G*C*T*+ | 1465 | 19 |
| | TTTC | | T*+T*+C | | |
| 474 | ATTAAGAAACATTGG | A57472Hi | +A*+T*+T*A*A*G*A*A*A*C*A*T*T*G*G*C*+ | 1466 | 19 |
| | CTTT | | T*+T*+T | | |
| 475 | CTTTATTATGCCTCCA | A57473Hi | +C*+T*+T*T*A*T*T*A*T*G*C*C*T*C*C*A*+ | 1719 | 19 |
| | TCT | | T*+C*+T | | |
| 476 | GCATCTTTATTATGC | A57474Hi | +G*+C*+A*T*C*T*T*T*A*T*T*A*T*G*C*C*+ | 1723 | 19 |
| | CTCC | | T*+C*+C | | |
| 477 | ACAGCATCTTTATTAT | A57475Hi | +A*+C*+A*G*C*A*T*C*T*T*T*A*T*T*A*T*+ | 1726 | 19 |
| | GCC | | G*+C*+C | | |
| 478 | TCCGACTTTGAGTCT | A57476Hi | +T*+C*+C*G*A*C*T*T*T*G*A*G*T*C*T*T*+ | 1748 | 19 |
| | TAAA | | A*+A*+A | | |
| 479 | TTCCGACTTTGAGTC | A57477Hi | +T*+T*+C*C*G*A*C*T*T*T*G*A*G*T*C*T*+ | 1749 | 19 |
| | TTAA | | T*+A*+A | | |
| 480 | AAATTCCGACTTTGA | A57478Hi | +A*+A*+A*T*T*C*C*G*A*C*T*T*T*G*A*+G* | 1753 | 18 |
| | GTC | | +T*+C | | |
| 481 | AAATTCCGACTTTGA | A57479Hi | +A*+A*+A*T*T*C*C*G*A*C*T*T*T*G*+A*+G | 1754 | 17 |
| | GT | | *+T | | |
| 482 | CAGGAAGCACAATAG | A57480Hi | +C*+A*+G*G*A*A*G*C*A*C*A*A*T*A*G*G* | 1803 | 19 |
| | GCCA | | +C*+C*+A | | |
| 483 | GAGCAGGAAGCACAA | A57481Hi | +G*+A*+G*C*A*G*G*A*A*G*C*A*C*A*A*T* | 1806 | 19 |
| | TAGG | | +A*+G*+G | | |
| 484 | AGAGCAGGAAGCACA | A57482Hi | +A*+G*+A*G*C*A*G*G*A*A*G*C*A*C*A*A* | 1807 | 19 |
| | ATAG | | +T*+A*+G | | |
| 485 | CAGAGCAGGAAGCAC | A57483Hi | +C*+A*+G*A*G*C*A*G*G*A*A*G*C*A*C*A* | 1808 | 19 |
| | AATA | | +A*+T*+A | | |
| 486 | GGTGCAGAGCAGGAA | A57484Hi | +G*+G*+T*G*C*A*G*A*G*C*A*G*G*A*A*G* | 1812 | 19 |
| | GCAC | | +C*+A*+C | | |
| 487 | GACAAGTATGCTCAG | A57485Hi | +G*+A*+C*A*A*G*T*A*T*G*C*T*C*A*G*+G* | 2065 | 18 |
| | GCT | | +C*+T | | |
| 488 | CAAGCTCTTTGATCC | A57486Hi | +C*+A*+A*G*C*T*C*T*T*T*G*A*T*C*C*T*+ | 3912 | 19 |
| | TCAG | | C*+A*+G | | |
| 489 | TGTCACAAGCTCTTT | A57487Hi | +T*+G*+T*C*A*C*A*A*G*C*T*C*T*T*T*G*+ | 3917 | 19 |
| | GATC | | A*+T*+C | | |
| 490 | TGTCACAAGCTCTTT | A57488Hi | +T*+G*+T*C*A*C*A*A*G*C*T*C*T*T*T*+G* | 3918 | 18 |
| | GAT | | +A*+T | | |
| 491 | GTATGATGGAAAGAG | A57489Hi | +G*+T*+A*T*G*A*T*G*G*A*A*A*G*A*G*A*+ | 3967 | 19 |
| | ATTT | | T*+T*+T | | |
| 492 | TAGATTGTATGATGG | A57490Hi | +T*+A*+G*A*T*T*G*T*A*T*G*A*T*G*G*A*+ | 3973 | 19 |
| | AAAG | | A*+A*+G | | |
| 493 | TAGTAGATTGTATGA | A57491Hi | +T*+A*+G*T*A*G*A*T*T*G*T*A*T*G*A*+T* | 3977 | 18 |
| | TGG | | +G*+G | | |
| 494 | CTAGTAGATTGTATG | A57492Hi | +C*+T*+A*G*T*A*G*A*T*T*G*T*A*T*G*+A* | 3978 | 18 |
| | ATG | | +T*+G | | |
| 495 | AGCTAGTAGATTGTA | A57493Hi | +A*+G*+C*T*A*G*T*A*G*A*T*T*G*T*A*+T* | 3980 | 18 |
| | TGA | | +G*+A | | |
| 496 | CAGCTAGTAGATTGT | A57494Hi | +C*+A*+G*C*T*A*G*T*A*G*A*T*T*G*T*A*+ | 3980 | 19 |
| | ATGA | | T*+G*+A | | |
| 497 | GCAGCTAGTAGATTG | A57495Hi | +G*+C*+A*G*C*T*A*G*T*A*G*A*T*T*G*+T* | 3982 | 18 |
| | TAT | | +A*+T | | |
| 498 | AGCAGCTAGTAGATT | A57496Hi | +A*+G*+C*A*G*C*T*A*G*T*A*G*A*T*T*+G* | 3983 | 18 |
| | GTA | | +T*+A | | |
| 499 | ATTGATGTCATTGTA | A57497Hi | +A*+T*+T*G*A*T*G*T*C*A*T*T*G*T*A*+C* | 4070 | 18 |
| | CCT | | +C*+T | | |
| 500 | CTTACAGTAATTATA | A57498Hi | +C*+T*+T*A*C*A*G*T*A*A*T*T*A*T*A*C*+ | 4193 | 19 |
| | CTAA | | T*+A*+A | | |
| 501 | AAAGGATACTTACAG | A57499Hi | +A*+A*+A*G*G*A*T*A*C*T*T*A*C*A*G*T*+ | 4201 | 19 |
| | TAAT | | A*+A*+T | | |
| 502 | TAGAAACAAACCTTA | A57500Hi | +T*+A*+G*A*A*A*C*A*A*A*C*C*T*T*A*A*+ | 4346 | 19 |
| | ATCT | | T*+C*+T | | |
| 503 | AATGGCATCTAGAAA | A57501Hi | +A*+A*+T*G*G*C*A*T*C*T*A*G*A*A*A*C*+ | 4355 | 19 |
| | CAAA | | A*+A*+A | | |
| 504 | TTAAATGGCATCTAG | A57502Hi | +T*+T*+A*A*A*T*G*G*C*A*T*C*T*A*G*A*+ | 4358 | 19 |
| | AAAC | | A*+A*+C | | |
| 505 | ATATCATGTTAAATG | A57503Hi | +A*+T*+A*T*C*A*T*G*T*T*A*A*A*T*G*G*+ | 4366 | 19 |
| | GCAT | | C*+A*+T | | |
| 506 | GAATATCATGTTAAA | A57504Hi | +G*+A*+A*T*A*T*C*A*T*G*T*T*A*A*A*T*+ | 4368 | 19 |
| | TGGC | | G*+G*+C | | |
| 507 | CTAAAGACTGTCATT | A57505Hi | +C*+T*+A*A*A*G*A*C*T*G*T*C*A*T*T*T*+ | 4440 | 19 |
| | TATG | | A*+T*+G | | |
| 508 | AAATTTAGGAGCACA | A57506Hi | +A*+A*+A*T*T*T*A*G*G*A*G*C*A*C*A*G*+ | 4512 | 19 |
| | GTGA | | T*+G*+A | | |
| 509 | AACTCAAGCATGTAA | A57507Hi | +A*+A*+C*T*C*A*A*G*C*A*T*G*T*A*A*T*+ | 4636 | 19 |
| | TTAA | | T*+A*+A | | |
| 510 | CAATACAATTAAATT | A57508Hi | +C*+A*+A*T*A*C*A*A*T*T*A*A*A*T*T*G*+ | 4682 | 19 |
| | GGTA | | G*+T*+A | | |
| 511 | ACAATACAATTAAAT | A57509Hi | +A*+C*+A*A*T*A*C*A*A*T*T*A*A*A*T*T*+ | 4683 | 19 |
| | TGGT | | G*+G*+T | | |
| 512 | TACAATACAATTAAA | A57510Hi | +T*+A*+C*A*A*T*A*C*A*A*T*T*A*A*A*T*+ | 4684 | 19 |
| | TTGG | | T*+G*+G | | |
| 513 | TAACAATTGATCCAT | A57511Hi | +T*+A*+A*C*A*A*T*T*G*A*T*C*C*A*T*T*+ | 4757 | 19 |
| | TAAT | | A*+A*+T | | |
| 514 | AGCAAAGCAGAGGTA | A57512Hi | +A*+G*+C*A*A*A*G*C*A*G*A*G*G*T*A*A* | 4770 | 19 |
| | ACAA | | +C*+A*+A | | |
| 515 | TACATGGAACTTCTG | A57513Hi | +T*+A*+C*A*T*G*G*A*A*C*T*T*C*T*G*T*+ | 4806 | 19 |
| | TGAT | | G*+A*+T | | |
| 516 | AGGCAACTGAGCTCT | A57514Hi | +A*+G*+G*C*A*A*C*T*G*A*G*C*T*C*T*C*+ | 4828 | 19 |
| | CCAG | | C*+A*+G | | |
| 517 | CATAGGATGCCAAAC | A57515Hi | +C*+A*+T*A*G*G*A*T*G*C*C*A*A*A*C*T*+ | 4937 | 19 |
| | TCAT | | C*+A*+T | | |
| 518 | TCACATAGGATGCCA | A57516Hi | +T*+C*+A*C*A*T*A*G*G*A*T*G*C*C*A*+A* | 4941 | 18 |
| | AAC | | +A*+C | | |
| 519 | TCTGAACAGCTTCTG | A57517Hi | +T*+C*+T*G*A*A*C*A*G*C*T*T*C*T*G*G*+ | 4989 | 19 |
| | GTGG | | T*+G*+G | | |
| 520 | TTCTGAACAGCTTCT | A57518Hi | +T*+T*+C*T*G*A*A*C*A*G*C*T*T*C*T*G*+ | 4990 | 19 |
| | GGTG | | G*+T*+G | | |
| 521 | ATTCTGAACAGCTTC | A57519Hi | +A*+T*+T*C*T*G*A*A*C*A*G*C*T*T*C*T*+ | 4991 | 19 |
| | TGGT | | G*+G*+T | | |
| 522 | AACATTCTGAACAGC | A57520Hi | +A*+A*+C*A*T*T*C*T*G*A*A*C*A*G*C*T*+ | 4994 | 19 |
| | TTCT | | T*+C*+T | | |
| 523 | AATCCAGCTGTCATA | A57521Hi | +A*+A*+T*C*C*A*G*C*T*G*T*C*A*T*A*G*+ | 5075 | 19 |
| | GAAA | | A*+A*+A | | |
| 524 | ACAATCCAGCTGTCA | A57522Hi | +A*+C*+A*A*T*C*C*A*G*C*T*G*T*C*A*T*+ | 5077 | 19 |
| | TAGA | | A*+G*+A | | |
| 525 | ACAATCCAGCTGTCA | A57523Hi | +A*+C*+A*A*T*C*C*A*G*C*T*G*T*C*A*+T* | 5078 | 18 |
| | TAG | | +A*+G | | |
| 526 | CCACGTAAGCTGAAC | A57524Hi | +C*+C*+A*C*G*T*A*A*G*C*T*G*A*A*+C*+A | 5102 | 17 |
| | AG | | *+G | | |
| 527 | CCTCCACGTAAGCTG | A57525Hi | +C*+C*+T*C*C*A*C*G*T*A*A*G*C*T*+G*+A | 5105 | 17 |
| | AA | | *+A | | |
| 528 | CTCCTCCACGTAAGC | A57526Hi | +C*+T*+C*C*T*C*C*A*C*G*T*A*A*G*C*T*+ | 5105 | 19 |
| | TGAA | | G*+A*+A | | |
| 529 | TCCTCCACGTAAGCT | A57527Hi | +T*+C*+C*T*C*C*A*C*G*T*A*A*G*C*T*+G* | 5105 | 18 |
| | GAA | | +A*+A | | |
| 530 | CTCCTCCACGTAAGC | A57528Hi | +C*+T*+C*C*T*C*C*A*C*G*T*A*A*G*C*+T* | 5106 | 18 |
| | TGA | | +G*+A | | |
| 531 | CCTCCTCCACGTAAG | A57529Hi | +C*+C*+T*C*C*T*C*C*A*C*G*T*A*A*G*+C* | 5107 | 18 |
| | CTG | | +T*+G | | |
| 532 | CTCCTCCACGTAAGC | A57530Hi | +C*+T*+C*C*T*C*C*A*C*G*T*A*A*G*+C*+T | 5107 | 17 |
| | TG | | *+G | | |
| 533 | TCCTCCTCCACGTAA | A57531Hi | +T*+C*+C*T*C*C*T*C*C*A*C*G*T*A*A*G*+ | 5107 | 19 |
| | GCTG | | C*+T*+G | | |
| 534 | GACATCCTCCTCCAC | A57532Hi | +G*+A*+C*A*T*C*C*T*C*C*T*C*C*A*C*G*+ | 5111 | 19 |
| | GTAA | | T*+A*+A | | |
| 535 | TAGGACATCCTCCTC | A57533Hi | +T*+A*+G*G*A*C*A*T*C*C*T*C*C*T*C*C*+ | 5114 | 19 |
| | CACG | | A*+C*+G | | |
| 536 | TAGGACATCCTCCTC | A57534Hi | +T*+A*+G*G*A*C*A*T*C*C*T*C*C*T*C*+C* | 5115 | 18 |
| | CAC | | +A*+C | | |
| 537 | GTAGGACATCCTCCT | A57535Hi | +G*+T*+A*G*G*A*C*A*T*C*C*T*C*C*T*C*+ | 5115 | 19 |
| | CCAC | | C*+A*+C | | |
| 538 | AGTAGGACATCCTCC | A57536Hi | +A*+G*+T*A*G*G*A*C*A*T*C*C*T*C*C*T*+ | 5116 | 19 |
| | TCCA | | C*+C*+A | | |
| 539 | AAGTAGGACATCCTC | A57537Hi | +A*+A*+G*T*A*G*G*A*C*A*T*C*C*T*C*C*+ | 5117 | 19 |
| | CTCC | | T*+C*+C | | |
| 540 | ATAATGGCACAAGTT | A57538Hi | +A*+T*+A*A*T*G*G*C*A*C*A*A*G*T*T*T*+ | 5331 | 19 |
| | TCTT | | C*+T*+T | | |
| 541 | CCAATAAGGAATAAT | A57539Hi | +C*+C*+A*A*T*A*A*G*G*A*A*T*A*A*T*G*+ | 5341 | 19 |
| | GGCA | | G*+C*+A | | |
| 542 | GAGTTGAATGACAAG | A57540Hi | +G*+A*+G*T*T*G*A*A*T*G*A*C*A*A*G*G*+ | 5375 | 19 |
| | GTGA | | T*+G*+A | | |
| 543 | GCAAGGAGTTGAATG | A57541Hi | +G*+C*+A*A*G*G*A*G*T*T*G*A*A*T*G*A*+ | 5380 | 19 |
| | ACAA | | C*+A*+A | | |
| 544 | GATGCATAAGTAAAG | A57542Hi | +G*+A*+T*G*C*A*T*A*A*G*T*A*A*A*G*T*+ | 5404 | 19 |
| | TTGA | | T*+G*+A | | |
| 545 | GGATGCATAAGTAAA | A57543Hi | +G*+G*+A*T*G*C*A*T*A*A*G*T*A*A*A*G*+ | 5405 | 19 |
| | GTTG | | T*+T*+G | | |
| 546 | GATAAGGCTGGATGC | A57544Hi | +G*+A*+T*A*A*G*G*C*T*G*G*A*T*G*C*A*+ | 5414 | 19 |
| | ATAA | | T*+A*+A | | |
| 547 | GAGAGTAGGCTGCTA | A57545Hi | +G*+A*+G*A*G*T*A*G*G*C*T*G*C*T*A*+C* | 5469 | 18 |
| | CAT | | +A*+T | | |
| 548 | GAGGAGAGTAGGCTG | A57546Hi | +G*+A*+G*G*A*G*A*G*T*A*G*G*C*T*G*C* | 5471 | 19 |
| | CTAC | | +T*+A*+C | | |
| 549 | TGAGGAGAGTAGGCT | A57547Hi | +T*+G*+A*G*G*A*G*A*G*T*A*G*G*C*T*G* | 5472 | 19 |
| | GCTA | | +C*+T*+A | | |
| 550 | GTGAGGAGAGTAGGC | A57548Hi | +G*+T*+G*A*G*G*A*G*A*G*T*A*G*G*C*T* | 5473 | 19 |
| | TGCT | | +G*+C*+T | | |
| 551 | GTTGAAATTACTCAG | A57549Hi | +G*+T*+T*G*A*A*A*T*T*A*C*T*C*A*G*A*+ | 5547 | 19 |
| | AGAA | | G*+A*+A | | |
| 552 | TGTGAGTTGAAATTA | A57550Hi | +T*+G*+T*G*A*G*T*T*G*A*A*A*T*T*A*C*+ | 5552 | 19 |
| | CTCA | | T*+C*+A | | |
| 553 | AGTGTGAGTTGAAAT | A57551Hi | +A*+G*+T*G*T*G*A*G*T*T*G*A*A*A*T*T*+ | 5554 | 19 |
| | TACT | | A*+C*+T | | |
| 554 | TAGAAATACTGAAAG | A57552Hi | +T*+A*+G*A*A*A*T*A*C*T*G*A*A*A*G*T*+ | 5577 | 19 |
| | TAAG | | A*+A*+G | | |
| 555 | ATAGAAATACTGAAA | A57553Hi | +A*+T*+A*G*A*A*A*T*A*C*T*G*A*A*A*G*+ | 5578 | 19 |
| | GTAA | | T*+A*+A | | |
| 556 | AAGTGTGATGTGTAA | A57554Hi | +A*+A*+G*T*G*T*G*A*T*G*T*G*T*A*A*+T* | 5608 | 18 |
| | TAA | | +A*+A | | |
| 557 | CTAAGTGTGATGTGT | A57555Hi | +C*+T*+A*A*G*T*G*T*G*A*T*G*T*G*T*+A* | 5610 | 18 |
| | AAT | | +A*+T | | |
| 558 | CTATACTTGACATGG | A57556Hi | +C*+T*+A*T*A*C*T*T*G*A*C*A*T*G*G*+T* | 5689 | 18 |
| | TCA | | +C*+A | | |
| 559 | AACTATACTTGACAT | A57557Hi | +A*+A*+C*T*A*T*A*C*T*T*G*A*C*A*T*G*+ | 5690 | 19 |
| | GGTC | | G*+T*+C | | |
| 560 | ACTATACTTGACATG | A57558Hi | +A*+C*+T*A*T*A*C*T*T*G*A*C*A*T*G*+G* | 5690 | 18 |
| | GTC | | +T*+C | | |
| 561 | GAACTATACTTGACA | A57559Hi | +G*+A*+A*C*T*A*T*A*C*T*T*G*A*C*A*T*+ | 5691 | 19 |
| | TGGT | | G*+G*+T | | |
| 562 | GAACTATACTTGACA | A57560Hi | +G*+A*+A*C*T*A*T*A*C*T*T*G*A*C*A*+T* | 5692 | 18 |
| | TGG | | +G*+G | | |
| 563 | TATAGAACTATACTT | A57561Hi | +T*+A*+T*A*G*A*A*C*T*A*T*A*C*T*T*G*+ | 5695 | 19 |
| | GACA | | A*+C*+A | | |
| 564 | TATAGAACTATACTT | A57562Hi | +T*+A*+T*A*G*A*A*C*T*A*T*A*C*T*T*+G* | 5696 | 18 |
| | GAC | | +A*+C | | |
| 565 | CTATTATACTATAGA | A57563Hi | +C*+T*+A*T*T*A*T*A*C*T*A*T*A*G*A*A*+ | 5704 | 19 |
| | ACTA | | C*+T*+A | | |
| 566 | AATTGAAAGTCCTGA | A57564Hi | +A*+A*+T*T*G*A*A*A*G*T*C*C*T*G*A*T*+ | 5801 | 19 |
| | TTTC | | T*+T*+C | | |
| 567 | TTTACAAGATGCCAA | A57565Hi | +T*+T*+T*A*C*A*A*G*A*T*G*C*C*A*A*C*+ | 5849 | 19 |
| | CTTG | | T*+T*+G | | |
| 568 | TTACAAGATGCCAAC | A57566Hi | +T*+T*+A*C*A*A*G*A*T*G*C*C*A*A*C*+T* | 5849 | 18 |
| | TTG | | +T*+G | | |
| 569 | GTTTACAAGATGCCA | A57567Hi | +G*+T*+T*T*A*C*A*A*G*A*T*G*C*C*A*A*+ | 5850 | 19 |
| | ACTT | | C*+T*+T | | |
| 570 | CTCAGTCTGTTTACA | A57568Hi | +C*+T*+C*A*G*T*C*T*G*T*T*T*A*C*A*A*+ | 5858 | 19 |
| | AGAT | | G*+A*+T | | |
| 571 | ACTCAGTCTGTTTAC | A57569Hi | +A*+C*+T*C*A*G*T*C*T*G*T*T*T*A*C*A*+ | 5859 | 19 |
| | AAGA | | A*+G*+A | | |
| 572 | CTTCACTCAGTCTGT | A57570Hi | +C*+T*+T*C*A*C*T*C*A*G*T*C*T*G*T*T*+ | 5863 | 19 |
| | TTAC | | T*+A*+C | | |
| 573 | ATCTTCACTCAGTCT | A57571Hi | +A*+T*+C*T*T*C*A*C*T*C*A*G*T*C*T*G*+ | 5865 | 19 |
| | GTTT | | T*+T*+T | | |
| 574 | TATATCTTCACTCAGT | A57572Hi | +T*+A*+T*A*T*C*T*T*C*A*C*T*C*A*G*T*+ | 5868 | 19 |
| | CTG | | C*+T*+G | | |
| 575 | CTTTCACATTTCGAA | A57573Hi | +C*+T*+T*T*C*A*C*A*T*T*T*C*G*A*A*A*+ | 5914 | 19 |
| | ATAA | | T*+A*+A | | |
| 576 | GCTCTTTCACATTTC | A57574Hi | +G*+C*+T*C*T*T*T*C*A*C*A*T*T*T*C*G*+ | 5917 | 19 |
| | GAAA | | A*+A*+A | | |
| 577 | TAGTAGGCTCTTTCA | A57575Hi | +T*+A*+G*T*A*G*G*C*T*C*T*T*T*C*A*C*+ | 5923 | 19 |
| | CATT | | A*+T*+T | | |
| 578 | TAGTAGGCTCTTTCA | A57576Hi | +T*+A*+G*T*A*G*G*C*T*C*T*T*T*C*A*+C* | 5924 | 18 |
| | CAT | | +A*+T | | |
| 579 | CCTAGTAGGCTCTTT | A57577Hi | +C*+C*+T*A*G*T*A*G*G*C*T*C*T*T*T*C*+ | 5925 | 19 |
| | CACA | | A*+C*+A | | |
| 580 | CTAGTAGGCTCTTTC | A57578Hi | +C*+T*+A*G*T*A*G*G*C*T*C*T*T*T*C*+A* | 5925 | 18 |
| | ACA | | +C*+A | | |
| 581 | CTAGTAGGCTCTTTC | A57579Hi | +C*+T*+A*G*T*A*G*G*C*T*C*T*T*T*+C*+A | 5926 | 17 |
| | AC | | *+C | | |
| 582 | CCTAGTAGGCTCTTT | A57580Hi | +C*+C*+T*A*G*T*A*G*G*C*T*C*T*T*T*+C* | 5926 | 18 |
| | CAC | | +A*+C | | |
| 583 | ATTGATAAGGTCTAG | A57581Hi | +A*+T*+T*G*A*T*A*A*G*G*T*C*T*A*G*A*+ | 5954 | 19 |
| | AGAT | | G*+A*+T | | |
| 584 | TTCTCTAGAATGAAT | A57582Hi | +T*+T*+C*T*C*T*A*G*A*A*T*G*A*A*T*T*+ | 5967 | 19 |
| | TGAT | | G*+A*+T | | |
| 585 | GTAGAACTCCTCAAT | A57583Hi | +G*+T*+A*G*A*A*C*T*C*C*T*C*A*A*T*+G* | 5997 | 18 |
| | GGC | | +G*+C | | |
| 586 | GGTAGAACTCCTCAA | A57584Hi | +G*+G*+T*A*G*A*A*C*T*C*C*T*C*A*A*T*+ | 5997 | 19 |
| | TGGC | | G*+G*+C | | |
| 587 | GAGGTAGAACTCCTC | A57585Hi | +G*+A*+G*G*T*A*G*A*A*C*T*C*C*T*C*A*+ | 5999 | 19 |
| | AATG | | A*+T*+G | | |
| 588 | AGGAGGTAGAACTCC | A57586Hi | +A*+G*+G*A*G*G*T*A*G*A*A*C*T*C*C*T*+ | 6001 | 19 |
| | TCAA | | C*+A*+A | | |
| 589 | ATAGACAGGAGGTAG | A57587Hi | +A*+T*+A*G*A*C*A*G*G*A*G*G*T*A*G*A* | 6007 | 19 |
| | AACT | | +A*+C*+T | | |
| 590 | AATAGAGGCAAGAAA | A57588Hi | +A*+A*+T*A*G*A*G*G*C*A*A*G*A*A*A*+C* | 6085 | 18 |
| | CGT | | +G*+T | | |
| 591 | AAGCTAGAATAATAG | A57589Hi | +A*+A*+G*C*T*A*G*A*A*T*A*A*T*A*G*+A* | 6095 | 18 |
| | AGG | | +G*+G | | |
| 592 | TGAAGCTAGAATAAT | A57590Hi | +T*+G*+A*A*G*C*T*A*G*A*A*T*A*A*T*A*+ | 6096 | 19 |
| | AGAG | | G*+A*+G | | |
| 593 | TAAGTTATCAGAATG | A57591Hi | +T*+A*+A*G*T*T*A*T*C*A*G*A*A*T*G*G*+ | 6127 | 19 |
| | GCTG | | C*+T*+G | | |
| 594 | GTAAGTTATCAGAAT | A57592Hi | +G*+T*+A*A*G*T*T*A*T*C*A*G*A*A*T*+G* | 6129 | 18 |
| | GGC | | +G*+C | | |
| 595 | TGTAAGTTATCAGAA | A57593Hi | +T*+G*+T*A*A*G*T*T*A*T*C*A*G*A*A*+T* | 6130 | 18 |
| | TGG | | +G*+G | | |
| 596 | GTACCAAGATTATTA | A57594Hi | +G*+T*+A*C*C*A*A*G*A*T*T*A*T*T*A*A*+ | 6639 | 19 |
| | AGTG | | G*+T*+G | | |
| 597 | GTTGTACCAAGATTA | A57595Hi | +G*+T*+T*G*T*A*C*C*A*A*G*A*T*T*A*+T* | 6643 | 18 |
| | TTA | | +T*+A | | |
| 598 | AGGTTGTACCAAGAT | A57596Hi | +A*+G*+G*T*T*G*T*A*C*C*A*A*G*A*T*T*+ | 6644 | 19 |
| | TATT | | A*+T*+T | | |
| 599 | TTTAGAGGTTGTACC | A57597Hi | +T*+T*+T*A*G*A*G*G*T*T*G*T*A*C*C*+A* | 6650 | 18 |
| | AAG | | +A*+G | | |
| 600 | AGGATACACCTTTCT | A57598Hi | +A*+G*+G*A*T*A*C*A*C*C*T*T*T*C*T*C*+ | 7079 | 19 |
| | CCAA | | C*+A*+A | | |
| 601 | TAGGATACACCTTTC | A57599Hi | +T*+A*+G*G*A*T*A*C*A*C*C*T*T*T*C*T*+ | 7080 | 19 |
| | TCCA | | C*+C*+A | | |
| 602 | AGTCAAACATGAATC | A57600Hi | +A*+G*+T*C*A*A*A*C*A*T*G*A*A*T*C*T*+ | 7201 | 19 |
| | TCTT | | C*+T*+T | | |
| 603 | TTGCACAGAAGTGAC | A57601H | +T*+T*+G*C*A*C*A*G*A*A*G*T*G*A*C*C*+ | 7282 | 19 |
| | CAAC | | A*+A*+C | | |
| 604 | CTATTAATTTGCACA | A57602H | +C*+T*+A*T*T*A*A*T*T*T*G*C*A*C*A*G*+ | 7290 | 19 |
| | GAAG | | A*+A*+G | | |
| 605 | TTAAGCTTTGCATTG | A57603H | +T*+T*+A*A*G*C*T*T*T*G*C*A*T*T*G*T*+ | 7320 | 19 |
| | TACC | | A*+C*+C | | |
| 606 | ATTAAGCTTTGCATT | A57604H | +A*+T*+T*A*A*G*C*T*T*T*G*C*A*T*T*G*+ | 7321 | 19 |
| | GTAC | | T*+A*+C | | |
| 607 | CCCATTAAGCTTTGC | A57605H | +C*+C*+C*A*T*T*A*A*G*C*T*T*T*G*C*A*+ | 7324 | 19 |
| | ATTG | | T*+T*+G | | |
| 608 | TCCCATTAAGCTTTG | A57606H | +T*+C*+C*C*A*T*T*A*A*G*C*T*T*T*G*C*+ | 7325 | 19 |
| | CATT | | A*+T*+T | | |
| 609 | GTCCCATTAAGCTTT | A57607H | +G*+T*+C*C*C*A*T*T*A*A*G*C*T*T*T*G*+ | 7326 | 19 |
| | GCAT | | C*+A*+T | | |
| 610 | GGTCCCATTAAGCTT | A57608H | +G*+G*+T*C*C*C*A*T*T*A*A*G*C*T*T*T*+ | 7327 | 19 |
| | TGCA | | G*+C*+A | | |
| 611 | GGGTCCCATTAAGCT | A57609H | +G*+G*+G*T*C*C*C*A*T*T*A*A*G*C*T*T*+ | 7328 | 19 |
| | TTGC | | T*+G*+C | | |
| 612 | GGGTCCCATTAAGCT | A57610H | +G*+G*+G*T*C*C*C*A*T*T*A*A*G*C*T*+T* | 7329 | 18 |
| | TTG | | +T*+G | | |
| 613 | GGGGTCCCATTAAGC | A57611H | +G*+G*+G*G*T*C*C*C*A*T*T*A*A*G*C*T*+ | 7329 | 19 |
| | TTTG | | T*+T*+G | | |
| 614 | ATCCTGGGGTCCCAT | A57612H | +A*+T*+C*C*T*G*G*G*G*T*C*C*C*A*T*T*+ | 7334 | 19 |
| | TAAG | | A*+A*+G | | |
| 615 | CCAACATCCTGGGGT | A57613H | +C*+C*+A*A*C*A*T*C*C*T*G*G*G*G*T*C*+ | 7339 | 19 |
| | CCCA | | C*+C*+A | | |
| 616 | TCATTTCCAACATCCT | A57614H | +T*+C*+A*T*T*T*C*C*A*A*C*A*T*C*C*T*+ | 7345 | 19 |
| | GGG | | G*+G*+G | | |
| 617 | ATCATTTCCAACATC | A57615H | +A*+T*+C*A*T*T*T*C*C*A*A*C*A*T*C*C*+ | 7346 | 19 |
| | CTGG | | T*+G*+G | | |
| 618 | CCAATCATTTCCAAC | A57616H | +C*+C*+A*A*T*C*A*T*T*T*C*C*A*A*C*A*+ | 7349 | 19 |
| | ATCC | | T*+C*+C | | |
| 619 | CCCAATCATTTCCAA | A57617H | +C*+C*+C*A*A*T*C*A*T*T*T*C*C*A*A*C*+ | 7350 | 19 |
| | CATC | | A*+T*+C | | |
| 620 | TTCCCAATCATTTCCA | A57618H | +T*+T*+C*C*C*A*A*T*C*A*T*T*T*C*C*A*+ | 7352 | 19 |
| | ACA | | A*+C*+A | | |
| 621 | CTTCCCAATCATTTCC | A57619H | +C*+T*+T*C*C*C*A*A*T*C*A*T*T*T*C*C*+ | 7353 | 19 |
| | AAC | | A*+A*+C | | |
| 622 | GACTTCCCAATCATT | A57620H | +G*+A*+C*T*T*C*C*C*A*A*T*C*A*T*T*T*+ | 7355 | 19 |
| | TCCA | | C*+C*+A | | |
| 623 | GGACTTCCCAATCAT | A57621H | +G*+G*+A*C*T*T*C*C*C*A*A*T*C*A*T*T*+ | 7356 | 19 |
| | TTCC | | T*+C*+C | | |
| 624 | AGGACTTCCCAATCA | A57622H | +A*+G*+G*A*C*T*T*C*C*C*A*A*T*C*A*T*+ | 7357 | 19 |
| | TTTC | | T*+T*+C | | |
| 625 | AAGGACTTCCCAATC | A57623H | +A*+A*+G*G*A*C*T*T*C*C*C*A*A*T*C*A*+ | 7358 | 19 |
| | ATTT | | T*+T*+T | | |
| 626 | GAACACCAAGGACTT | A57624H | +G*+A*+A*C*A*C*C*A*A*G*G*A*C*T*T*C*+ | 7365 | 19 |
| | CCCA | | C*+C*+A | | |
| 627 | TGAACACCAAGGACT | A57625H | +T*+G*+A*A*C*A*C*C*A*A*G*G*A*C*T*T*+ | 7366 | 19 |
| | TCCC | | C*+C*+C | | |
| 628 | TTACATATCTATCATA | A57626Hi | +T*+T*+A*C*A*T*A*T*C*T*A*T*C*A*T*A*+ | 7390 | 19 |
| | CTT | | C*+T*+T | | |
| 629 | GTAGCCTTGGATTGC | A57627Hi | +G*+T*+A*G*C*C*T*T*G*G*A*T*T*G*C*+A* | 7467 | 18 |
| | AAA | | +A*+A | | |
| 630 | AGTAGCCTTGGATTG | A57628Hi | +A*+G*+T*A*G*C*C*T*T*G*G*A*T*T*G*C*+ | 7467 | 19 |
| | CAAA | | A*+A*+A | | |
| 631 | GTAGCCTTGGATTGC | A57629Hi | +G*+T*+A*G*C*C*T*T*G*G*A*T*T*G*+C*+A | 7468 | 17 |
| | AA | | *+A | | |
| 632 | AGTAGCCTTGGATTG | A57630Hi | +A*+G*+T*A*G*C*C*T*T*G*G*A*T*T*G*+C* | 7468 | 18 |
| | CAA | | +A*+A | | |
| 633 | AAATCATTGACTCAA | A57631Hi | +A*+A*+A*T*C*A*T*T*G*A*C*T*C*A*A*G*+ | 7516 | 19 |
| | GGAA | | G*+A*+A | | |
| 634 | CTCTATAAATGTAAA | A57632Hi | +C*+T*+C*T*A*T*A*A*A*T*G*T*A*A*A*T*+ | 7528 | 19 |
| | TCAT | | C*+A*+T | | |
| 635 | GAGTTTAAAGCTCTC | A57633Hi | +G*+A*+G*T*T*T*A*A*A*G*C*T*C*T*C*T*+ | 7540 | 19 |
| | TATA | | A*+T*+A | | |
| 636 | TCTGAGTTTAAAGCT | A57634Hi | +T*+C*+T*G*A*G*T*T*T*A*A*A*G*C*T*C*+ | 7543 | 19 |
| | CTCT | | T*+C*+T | | |
| 637 | TAAATCAAACTTCTG | A57635Hi | +T*+A*+A*A*T*C*A*A*A*C*T*T*C*T*G*A*+ | 7554 | 19 |
| | AGTT | | G*+T*+T | | |
| 638 | TCTAAATCAAACTTCT | A57636Hi | +T*+C*+T*A*A*A*T*C*A*A*A*C*T*T*C*T*+ | 7556 | 19 |
| | GAG | | G*+A*+G | | |
| 639 | GCTTTCTAAATCAAA | A57637Hi | +G*+C*+T*T*T*C*T*A*A*A*T*C*A*A*A*C*+ | 7560 | 19 |
| | CTTC | | T*+T*+C | | |
| 640 | CTGACATGTTACCTT | A57638Hi | +C*+T*+G*A*C*A*T*G*T*T*A*C*C*T*T*A*+ | 7583 | 19 |
| | AAAT | | A*+A*+T | | |
| 641 | CTGACATGTTACCTT | A57639Hi | +C*+T*+G*A*C*A*T*G*T*T*A*C*C*T*T*+A* | 7584 | 18 |
| | AAA | | +A*+A | | |
| 642 | ATCAACTTTCAAATTA | A57640Hi | +A*+T*+C*A*A*C*T*T*T*C*A*A*A*T*T*A*+ | 7774 | 19 |
| | CTA | | C*+T*+A | | |
| 643 | CATCAACTTTCAAATT | A57641Hi | +C*+A*+T*C*A*A*C*T*T*T*C*A*A*A*T*T*+ | 7775 | 19 |
| | ACT | | A*+C*+T | | |
| 644 | CATTATGCTGCAAAC | A57642Hi | +C*+A*+T*T*A*T*G*C*T*G*C*A*A*A*C*T*+ | 7945 | 19 |
| | TGCA | | G*+C*+A | | |
| 645 | CATTATGCTGCAAAC | A57643Hi | +C*+A*+T*T*A*T*G*C*T*G*C*A*A*A*C*+T* | 7946 | 18 |
| | TGC | | +G*+C | | |
| 646 | GCTAAGGAAGTAGTC | A57644Hi | +G*+C*+T*A*A*G*G*A*A*G*T*A*G*T*C*+T* | 7974 | 18 |
| | TCA | | +C*+A | | |
| 647 | TGGCTAAGGAAGTAG | A57645Hi | +T*+G*+G*C*T*A*A*G*G*A*A*G*T*A*G*T*+ | 7975 | 19 |
| | TCTC | | C*+T*+C | | |
| 648 | TGGCTAAGGAAGTAG | A57646Hi | +T*+G*+G*C*T*A*A*G*G*A*A*G*T*A*G*+T* | 7976 | 18 |
| | TCT | | +C*+T | | |
| 649 | GCAACACATTTGTCA | A57647Hi | +G*+C*+A*A*C*A*C*A*T*T*T*G*T*C*A*A*+ | 8480 | 19 |
| | AAGA | | A*+G*+A | | |
| 650 | AGCAACACATTTGTC | A57648Hi | +A*+G*+C*A*A*C*A*C*A*T*T*T*G*T*C*A*+ | 8481 | 19 |
| | AAAG | | A*+A*+G | | |
| 651 | TAAATATGCTCTTTCC | A57649Hi | +T*+A*+A*A*T*A*T*G*C*T*C*T*T*T*C*C*+ | 8530 | 19 |
| | AAA | | A*+A*+A | | |
| 652 | ATTGTAAATATGCTC | A57650Hi | +A*+T*+T*G*T*A*A*A*T*A*T*G*C*T*C*T*+ | 8534 | 19 |
| | TTTC | | T*+T*+C | | |
| 653 | TGAGTAATTCCTCAT | A57651Hi | +T*+G*+A*G*T*A*A*T*T*C*C*T*C*A*T*C*+ | 8619 | 19 |
| | CAAA | | A*+A*+A | | |
| 654 | AGACTGGCTCATCTT | A57652Hi | +A*+G*+A*C*T*G*G*C*T*C*A*T*C*T*T*T*+ | 8705 | 19 |
| | TGGA | | G*+G*+A | | |
| 655 | ATAGACTGGCTCATC | A57653Hi | +A*+T*+A*G*A*C*T*G*G*C*T*C*A*T*C*T*+ | 8707 | 19 |
| | TTTG | | T*+T*+G | | |
| 656 | GAGGTTATAGACTGG | A57654Hi | +G*+A*+G*G*T*T*A*T*A*G*A*C*T*G*G*C*+ | 8713 | 19 |
| | CTCA | | T*+C*+A | | |
| 657 | TGAGGTTATAGACTG | A57655Hi | +T*+G*+A*G*G*T*T*A*T*A*G*A*C*T*G*G*+ | 8714 | 19 |
| | GCTC | | C*+T*+C | | |
| 658 | CTGAGGTTATAGACT | A57656Hi | +C*+T*+G*A*G*G*T*T*A*T*A*G*A*C*T*+G* | 8716 | 18 |
| | GGC | | +G*+C | | |
| 659 | GGAATATGACTAGAT | A57657Hi | +G*+G*+A*A*T*A*T*G*A*C*T*A*G*A*T*G*+ | 8748 | 19 |
| | GAGT | | A*+G*+T | | |
| 660 | AGGAATATGACTAGA | A57658Hi | +A*+G*+G*A*A*T*A*T*G*A*C*T*A*G*A*T*+ | 8749 | 19 |
| | TGAG | | G*+A*+G | | |
| 661 | GGAATATGACTAGAT | A57659Hi | +G*+G*+A*A*T*A*T*G*A*C*T*A*G*A*T*+G* | 8749 | 18 |
| | GAG | | +A*+G | | |
| 662 | ACAGGAATATGACTA | A57660Hi | +A*+C*+A*G*G*A*A*T*A*T*G*A*C*T*A*G*+ | 8751 | 19 |
| | GATG | | A*+T*+G | | |
| 663 | GGATGAACAAAGTGA | A57661Hi | +G*+G*+A*T*G*A*A*C*A*A*A*G*T*G*A*G* | 8843 | 19 |
| | GTGA | | +T*+G*+A | | |
| 664 | CTATCTTCTTCACAAC | A57662Hi | +C*+T*+A*T*C*T*T*C*T*T*C*A*C*A*A*C*+ | 8922 | 19 |
| | TGC | | T*+G*+C | | |
| 665 | TTGCATAATCTCCATT | A57663Hi | +T*+T*+G*C*A*T*A*A*T*C*T*C*C*A*T*T*+ | 9181 | 19 |
| | TGG | | T*+G*+G | | |
| 666 | ATCTCTAGTCTATTCC | A57664Hi | +A*+T*+C*T*C*T*A*G*T*C*T*A*T*T*C*C*+ | 9278 | 19 |
| | TGC | | T*+G*+C | | |
| 667 | ATTATTGATTGCTACT | A57665Hi | +A*+T*+T*A*T*T*G*A*T*T*G*C*T*A*C*T*+ | 9718 | 19 |
| | GTT | | G*+T*+T | | |
| 668 | AATTATTGATTGCTA | A57666Hi | +A*+A*+T*T*A*T*T*G*A*T*T*G*C*T*A*C*+ | 9719 | 19 |
| | CTGT | | T*+G*+T | | |
| 669 | GTTAGTTTGTGTATA | A57667Hi | +G*+T*+T*A*G*T*T*T*G*T*G*T*A*T*A*T*+ | 9761 | 19 |
| | TAAA | | A*+A*+A | | |
| 670 | ATGTTAGTTTGTGTA | A57668Hi | +A*+T*+G*T*T*A*G*T*T*T*G*T*G*T*A*T*+ | 9763 | 19 |
| | TATA | | A*+T*+A | | |
| 671 | GATGTTAGTTTGTGT | A57669Hi | +G*+A*+T*G*T*T*A*G*T*T*T*G*T*G*T*A*+ | 9764 | 19 |
| | ATAT | | T*+A*+T | | |
| 672 | GATGTTAGTTTGTGT | A57670Hi | +G*+A*+T*G*T*T*A*G*T*T*T*G*T*G*T*+A* | 9765 | 18 |
| | ATA | | +T*+A | | |
| 673 | AGATGTTAGTTTGTG | A57671Hi | +A*+G*+A*T*G*T*T*A*G*T*T*T*G*T*G*T*+ | 9765 | 19 |
| | TATA | | A*+T*+A | | |
| 674 | AAAGATGTTAGTTTG | A57672Hi | +A*+A*+A*G*A*T*G*T*T*A*G*T*T*T*G*T*+ | 9767 | 19 |
| | TGTA | | G*+T*+A | | |
| 675 | TTAGACATGTTGATA | A57673Hi | +T*+T*+A*G*A*C*A*T*G*T*T*G*A*T*A*T*+ | 9804 | 19 |
| | TATT | | A*+T*+T | | |
| 676 | ATCTTAGACATGTTG | A57674Hi | +A*+T*+C*T*T*A*G*A*C*A*T*G*T*T*G*A*+ | 9807 | 19 |
| | ATAT | | T*+A*+T | | |
| 677 | ATCTTAGACATGTTG | A57675Hi | +A*+T*+C*T*T*A*G*A*C*A*T*G*T*T*G*+A* | 9808 | 18 |
| | ATA | | +T*+A | | |
| 678 | TATCTTAGACATGTT | A57676Hi | +T*+A*+T*C*T*T*A*G*A*C*A*T*G*T*T*G*+ | 9808 | 19 |
| | GATA | | A*+T*+A | | |
| 679 | CTCTCATTGTATTTAA | A57677Hi | +C*+T*+C*T*C*A*T*T*G*T*A*T*T*T*A*A*+ | 10519 | 19 |
| | TTC | | T*+T*+C | | |
| 680 | ATTATGAACTGTTAG | A57678H | +A*+T*+T*A*T*G*A*A*C*T*G*T*T*A*G*C*+ | 10642 | 19 |
| | CATT | | A*+T*+T | | |
| 681 | CATTATGAACTGTTA | A57679H | +C*+A*+T*T*A*T*G*A*A*C*T*G*T*T*A*G*+ | 10643 | 19 |
| | GCAT | | C*+A*+T | | |
| 682 | CATTATGAACTGTTA | A57680H | +C*+A*+T*T*A*T*G*A*A*C*T*G*T*T*A*+G* | 10644 | 18 |
| | GCA | | +C*+A | | |
| 683 | GCATTATGAACTGTT | A57681H | +G*+C*+A*T*T*A*T*G*A*A*C*T*G*T*T*A*+ | 10644 | 19 |
| | AGCA | | G*+C*+A | | |
| 684 | GCATTATGAACTGTT | A57682H | +G*+C*+A*T*T*A*T*G*A*A*C*T*G*T*T*+A* | 10645 | 18 |
| | AGC | | +G*+C | | |
| 685 | GGCATTATGAACTGT | A57683H | +G*+G*+C*A*T*T*A*T*G*A*A*C*T*G*T*T*+ | 10645 | 19 |
| | TAGC | | A*+G*+C | | |
| 686 | GGCATTATGAACTGT | A57684H | +G*+G*+C*A*T*T*A*T*G*A*A*C*T*G*T*+T* | 10646 | 18 |
| | TAG | | +A*+G | | |
| 687 | TGGCATTATGAACTG | A57685H | +T*+G*+G*C*A*T*T*A*T*G*A*A*C*T*G*T*+ | 10646 | 19 |
| | TTAG | | T*+A*+G | | |
| 688 | CACTGGCATTATGAA | A57686H | +C*+A*+C*T*G*G*C*A*T*T*A*T*G*A*A*C*+ | 10649 | 19 |
| | CTGT | | T*+G*+T | | |
| 689 | TACACTGGCATTATG | A57687H | +T*+A*+C*A*C*T*G*G*C*A*T*T*A*T*G*A*+ | 10651 | 19 |
| | AACT | | A*+C*+T | | |
| 690 | TTACACTGGCATTAT | A57688H | +T*+T*+A*C*A*C*T*G*G*C*A*T*T*A*T*G*+ | 10652 | 19 |
| | GAAC | | A*+A*+C | | |
| 691 | GACAAGTTTACACTG | A57689H | +G*+A*+C*A*A*G*T*T*T*A*C*A*C*T*G*G*+ | 10659 | 19 |
| | GCAT | | C*+A*+T | | |
| 692 | GACAAGTTTACACTG | A57690H | +G*+A*+C*A*A*G*T*T*T*A*C*A*C*T*G*+G* | 10660 | 18 |
| | GCA | | +C*+A | | |
| 693 | TGACAAGTTTACACT | A57691H | +T*+G*+A*C*A*A*G*T*T*T*A*C*A*C*T*G*+ | 10660 | 19 |
| | GGCA | | G*+C*+A | | |
| 694 | TGACAAGTTTACACT | A57692H | +T*+G*+A*C*A*A*G*T*T*T*A*C*A*C*T*+G* | 10661 | 18 |
| | GGC | | +G*+C | | |
| 695 | AGGTCTTCAGTCCAA | A57693H | +A*+G*+G*T*C*T*T*C*A*G*T*C*C*A*A*+T* | 10680 | 18 |
| | TCA | | +C*+A | | |
| 696 | AAGGTCTTCAGTCCA | A57694H | +A*+A*+G*G*T*C*T*T*C*A*G*T*C*C*A*A*+ | 10680 | 19 |
| | ATCA | | T*+C*+A | | |
| 697 | TAAGGTCTTCAGTCC | A57695H | +T*+A*+A*G*G*T*C*T*T*C*A*G*T*C*C*A*+ | 10681 | 19 |
| | AATC | | A*+T*+C | | |
| 698 | CTAAGGTCTTCAGTC | A57696H | +C*+T*+A*A*G*G*T*C*T*T*C*A*G*T*C*C*+ | 10682 | 19 |
| | CAAT | | A*+A*+T | | |
| 699 | GATCTAAGGTCTTCA | A57697H | +G*+A*+T*C*T*A*A*G*G*T*C*T*T*C*A*G*+ | 10685 | 19 |
| | GTCC | | T*+C*+C | | |
| 700 | GAGATCTAAGGTCTT | A57698H | +G*+A*+G*A*T*C*T*A*A*G*G*T*C*T*T*C*+ | 10687 | 19 |
| | CAGT | | A*+G*+T | | |
| 701 | GGAGATCTAAGGTCT | A57699H | +G*+G*+A*G*A*T*C*T*A*A*G*G*T*C*T*T*+ | 10688 | 19 |
| | TCAG | | C*+A*+G | | |
| 702 | AGGAGATCTAAGGTC | A57700H | +A*+G*+G*A*G*A*T*C*T*A*A*G*G*T*C*T*+ | 10689 | 19 |
| | TTCA | | T*+C*+A | | |
| 703 | GAGGAGATCTAAGGT | A57701H | +G*+A*+G*G*A*G*A*T*C*T*A*A*G*G*T*C*+ | 10690 | 19 |
| | CTTC | | T*+T*+C | | |
| 704 | GGAGGAGATCTAAGG | A57702H | +G*+G*+A*G*G*A*G*A*T*C*T*A*A*G*G*T* | 10691 | 19 |
| | TCTT | | +C*+T*+T | | |
| 705 | AGGAGGAGATCTAAG | A57703H | +A*+G*+G*A*G*G*A*G*A*T*C*T*A*A*G*G* | 10692 | 19 |
| | GTCT | | +T*+C*+T | | |
| 706 | AGGAGGAGATCTAAG | A57704H | +A*+G*+G*A*G*G*A*G*A*T*C*T*A*A*G*+G | 10693 | 18 |
| | GTC | | *+T*+C | | |
| 707 | TTAGGACATTTAAAT | A57705Hi | +T*+T*+A*G*G*A*C*A*T*T*T*A*A*A*T*C*+ | 10837 | 19 |
| | CAAG | | A*+A*+G | | |
| 708 | ATGGCATACTCCAGA | A57706Hi | +A*+T*+G*G*C*A*T*A*C*T*C*C*A*G*A*+A* | 11171 | 18 |
| | ACA | | +C*+A | | |
| 709 | ATTATGGCATACTCC | A57707Hi | +A*+T*+T*A*T*G*G*C*A*T*A*C*T*C*C*A*+ | 11173 | 19 |
| | AGAA | | G*+A*+A | | |
| 710 | TGAAGCCTGGCATCT | A57708Hi | +T*+G*+A*A*G*C*C*T*G*G*C*A*T*C*T*A*+ | 11233 | 19 |
| | AGCA | | G*+C*+A | | |
| 711 | GTGAAGCCTGGCATC | A57709Hi | +G*+T*+G*A*A*G*C*C*T*G*G*C*A*T*C*T*+ | 11234 | 19 |
| | TAGC | | A*+G*+C | | |
| 712 | TGAAGCCTGGCATCT | A57710Hi | +T*+G*+A*A*G*C*C*T*G*G*C*A*T*C*T*+A* | 11234 | 18 |
| | AGC | | +G*+C | | |
| 713 | CTTGTGAAGCCTGGC | A57711Hi | +C*+T*+T*G*T*G*A*A*G*C*C*T*G*G*C*A*+ | 11237 | 19 |
| | ATCT | | T*+C*+T | | |
| 714 | ACTTGTGAAGCCTGG | A57712Hi | +A*+C*+T*T*G*T*G*A*A*G*C*C*T*G*G*C*+ | 11238 | 19 |
| | CATC | | A*+T*+C | | |
| 715 | CTTGTGAAGCCTGGC | A57713Hi | +C*+T*+T*G*T*G*A*A*G*C*C*T*G*G*C*+A* | 11238 | 18 |
| | ATC | | +T*+C | | |
| 716 | TACTTGTGAAGCCTG | A57714Hi | +T*+A*+C*T*T*G*T*G*A*A*G*C*C*T*G*G*+ | 11239 | 19 |
| | GCAT | | C*+A*+T | | |
| 717 | ACTTGTGAAGCCTGG | A57715Hi | +A*+C*+T*T*G*T*G*A*A*G*C*C*T*G*G*+C* | 11239 | 18 |
| | CAT | | +A*+T | | |
| 718 | TACTTGTGAAGCCTG | A57716Hi | +T*+A*+C*T*T*G*T*G*A*A*G*C*C*T*G*+G* | 11240 | 18 |
| | GCA | | +C*+A | | |
| 719 | TTACTTGTGAAGCCT | A57717Hi | +T*+T*+A*C*T*T*G*T*G*A*A*G*C*C*T*+G* | 11241 | 18 |
| | GGC | | +G*+C | | |
| 720 | TTTACTTGTGAAGCC | A57718Hi | +T*+T*+T*A*C*T*T*G*T*G*A*A*G*C*C*+T* | 11242 | 18 |
| | TGG | | +G*+G | | |
| 721 | GAACCTGACATGGAG | A57719Hi | +G*+A*+A*C*C*T*G*A*C*A*T*G*G*A*G*A*+ | 11393 | 19 |
| | AATC | | A*+T*+C | | |
| 722 | AAGGAACCTGACATG | A57720Hi | +A*+A*+G*G*A*A*C*C*T*G*A*C*A*T*G*G*+ | 11396 | 19 |
| | GAGA | | A*+G*+A | | |
| 723 | AAGGAACCTGACATG | A57721Hi | +A*+A*+G*G*A*A*C*C*T*G*A*C*A*T*G*+G* | 11397 | 18 |
| | GAG | | +A*+G | | |
| 724 | GAAGGAACCTGACAT | A57722Hi | +G*+A*+A*G*G*A*A*C*C*T*G*A*C*A*T*G*+ | 11397 | 19 |
| | GGAG | | G*+A*+G | | |
| 725 | GGAAGGAACCTGACA | A57723Hi | +G*+G*+A*A*G*G*A*A*C*C*T*G*A*C*A*T*+ | 11398 | 19 |
| | TGGA | | G*+G*+A | | |
| 726 | TTGCATCATTTCAAG | A57724Hi | +T*+T*+G*C*A*T*C*A*T*T*T*C*A*A*G*A*+ | 11460 | 19 |
| | ATTT | | T*+T*+T | | |
| 727 | AGTGTCCAGCACAGC | A57725Hi | +A*+G*+T*G*T*C*C*A*G*C*A*C*A*G*C*A*+ | 11489 | 19 |
| | AATG | | A*+T*+G | | |
| 728 | CCTACATGGAAGGAA | A57726Hi | +C*+C*+T*A*C*A*T*G*G*A*A*G*G*A*A*A*+ | 11573 | 19 |
| | ACTC | | C*+T*+C | | |
| 729 | TCTCTTAGCAGGACT | A57727Hi | +T*+C*+T*C*T*T*A*G*C*A*G*G*A*C*T*G*+ | 11689 | 19 |
| | GATG | | A*+T*+G | | |
| 730 | CTCTTAGCAGGACTG | A57728Hi | +C*+T*+C*T*T*A*G*C*A*G*G*A*C*T*G*+A* | 11689 | 18 |
| | ATG | | +T*+G | | |
| 731 | CATCTCTTAGCAGGA | A57729Hi | +C*+A*+T*C*T*C*T*T*A*G*C*A*G*G*A*C*+ | 11691 | 19 |
| | CTGA | | T*+G*+A | | |
| 732 | CCTCATACTCTCATCT | A57730Hi | +C*+C*+T*C*A*T*A*C*T*C*T*C*A*T*C*T*+ | 11732 | 19 |
| | TCC | | T*+C*+C | | |
| 733 | CCTCATACTCTCATCT | A57731Hi | +C*+C*+T*C*A*T*A*C*T*C*T*C*A*T*C*+T* | 11733 | 18 |
| | TC | | +T*+C | | |
| 734 | GTACTGTATGAGGCT | A57732Hi | +G*+T*+A*C*T*G*T*A*T*G*A*G*G*C*T*+A* | 11959 | 18 |
| | AAC | | +A*+C | | |
| 735 | AGTACTGTATGAGGC | A57733Hi | +A*+G*+T*A*C*T*G*T*A*T*G*A*G*G*C*T*+ | 11959 | 19 |
| | TAAC | | A*+A*+C | | |
| 736 | AGTACTGTATGAGGC | A57734Hi | +A*+G*+T*A*C*T*G*T*A*T*G*A*G*G*C*+T* | 11960 | 18 |
| | TAA | | +A*+A | | |
| 737 | GTAGTACTGTATGAG | A57735Hi | +G*+T*+A*G*T*A*C*T*G*T*A*T*G*A*G*+G* | 11962 | 18 |
| | GCT | | +C*+T | | |
| 738 | ACAAACAGCATCTAT | A57736Hi | +A*+C*+A*A*A*C*A*G*C*A*T*C*T*A*T*T*+ | 12012 | 19 |
| | TCTG | | C*+T*+G | | |
| 739 | CACTCCAAATATGCT | A57737Hi | +C*+A*+C*T*C*C*A*A*A*T*A*T*G*C*T*A*+ | 12146 | 19 |
| | AAAC | | A*+A*+C | | |
| 740 | TTCACTCCAAATATG | A57738Hi | +T*+T*+C*A*C*T*C*C*A*A*A*T*A*T*G*C*+ | 12148 | 19 |
| | CTAA | | T*+A*+A | | |
| 741 | TGTCTTCACTCCAAA | A57739Hi | +T*+G*+T*C*T*T*C*A*C*T*C*C*A*A*A*T*+ | 12152 | 19 |
| | TATG | | A*+T*+G | | |
| 742 | TTGTCTTCACTCCAA | A57740Hi | +T*+T*+G*T*C*T*T*C*A*C*T*C*C*A*A*A*+ | 12153 | 19 |
| | ATAT | | T*+A*+T | | |
| 743 | GTACATGTATCTCAT | A57741Hi | +G*+T*+A*C*A*T*G*T*A*T*C*T*C*A*T*C*+ | 12189 | 19 |
| | CAGC | | A*+G*+C | | |
| 744 | TAGTACATGTATCTC | A57742Hi | +T*+A*+G*T*A*C*A*T*G*T*A*T*C*T*C*A*+ | 12191 | 19 |
| | ATCA | | T*+C*+A | | |
| 745 | TATAGTACATGTATC | A57743Hi | +T*+A*+T*A*G*T*A*C*A*T*G*T*A*T*C*T*+ | 12193 | 19 |
| | TCAT | | C*+A*+T | | |
| 746 | CTATAGTACATGTAT | A57744Hi | +C*+T*+A*T*A*G*T*A*C*A*T*G*T*A*T*C*+ | 12194 | 19 |
| | CTCA | | T*+C*+A | | |
| 747 | TTGCATTCTCATCTA | A57745Hi | +T*+T*+G*C*A*T*T*C*T*C*A*T*C*T*A*G*+ | 12382 | 19 |
| | GAAT | | A*+A*+T | | |
| 748 | TTTGCATTCTCATCTA | A57746Hi | +T*+T*+T*G*C*A*T*T*C*T*C*A*T*C*T*A*+ | 12383 | 19 |
| | GAA | | G*+A*+A | | |
| 749 | GCACCTTCATTCCTA | A57747Hi | +G*+C*+A*C*C*T*T*C*A*T*T*C*C*T*A*T*+ | 12404 | 19 |
| | TTGT | | T*+G*+T | | |
| 750 | CAAGGCACCTTCATT | A57748Hi | +C*+A*+A*G*G*C*A*C*C*T*T*C*A*T*T*C*+ | 12408 | 19 |
| | CCTA | | C*+T*+A | | |
| 751 | ACAAGGCACCTTCAT | A57749Hi | +A*+C*+A*A*G*G*C*A*C*C*T*T*C*A*T*T*+ | 12409 | 19 |
| | TCCT | | C*+C*+T | | |
| 752 | AACAAGGCACCTTCA | A57750Hi | +A*+A*+C*A*A*G*G*C*A*C*C*T*T*C*A*T*+ | 12410 | 19 |
| | TTCC | | T*+C*+C | | |
| 753 | AACAAGGCACCTTCA | A57751Hi | +A*+A*+C*A*A*G*G*C*A*C*C*T*T*C*A*+T* | 12411 | 18 |
| | TTC | | +T*+C | | |
| 754 | TTTGAACAAGGCACC | A57752Hi | +T*+T*+T*G*A*A*C*A*A*G*G*C*A*C*C*T*+ | 12414 | 19 |
| | TTCA | | T*+C*+A | | |
| 755 | TATCTCCTACACATCT | A57753Hi | +T*+A*+T*C*T*C*C*T*A*C*A*C*A*T*C*T*+ | 12457 | 19 |
| | TGA | | T*+G*+A | | |
| 756 | CATCAATGATACCAT | A57754Hi | +C*+A*+T*C*A*A*T*G*A*T*A*C*C*A*T*A*+ | 12586 | 19 |
| | AATC | | A*+T*+C | | |
| 757 | TCATCAATGATACCA | A57755Hi | +T*+C*+A*T*C*A*A*T*G*A*T*A*C*C*A*T*+ | 12587 | 19 |
| | TAAT | | A*+A*+T | | |
| 758 | CTTCATCAATGATAC | A57756Hi | +C*+T*+T*C*A*T*C*A*A*T*G*A*T*A*C*C*+ | 12589 | 19 |
| | CATA | | A*+T*+A | | |
| 759 | CCTCTTAGACAACAT | A57757Hi | +C*+C*+T*C*T*T*A*G*A*C*A*A*C*A*T*+C* | 12653 | 18 |
| | CTG | | +T*+G | | |
| 760 | GCCTCTTAGACAACA | A57758Hi | +G*+C*+C*T*C*T*T*A*G*A*C*A*A*C*A*+T* | 12654 | 18 |
| | TCT | | +C*+T | | |
| 761 | TGCCTCTTAGACAAC | A57759Hi | +T*+G*+C*C*T*C*T*T*A*G*A*C*A*A*C*+A* | 12655 | 18 |
| | ATC | | +T*+C | | |
| 762 | GCCTCTTAGACAACA | A57760Hi | +G*+C*+C*T*C*T*T*A*G*A*C*A*A*C*+A*+T | 12655 | 17 |
| | TC | | *+C | | |
| 763 | TCTTATGCCTCTTAG | A57761Hi | +T*+C*+T*T*A*T*G*C*C*T*C*T*T*A*G*+A* | 12660 | 18 |
| | ACA | | +C*+A | | |
| 764 | TCTTATGCCTCTTAG | A57762Hi | +T*+C*+T*T*A*T*G*C*C*T*C*T*T*A*+G*+A | 12661 | 17 |
| | AC | | *+C | | |
| 765 | GAAATACATTCTTAT | A57763Hi | +G*+A*+A*A*T*A*C*A*T*T*C*T*T*A*T*+G* | 12669 | 18 |
| | GCC | | +C*+C | | |
| 766 | GCTCTAGATCTTTAT | A57764Hi | +G*+C*+T*C*T*A*G*A*T*C*T*T*T*A*T*A*+ | 12770 | 19 |
| | AAAT | | A*+A*+T | | |
| 767 | AAGCTCTAGATCTTT | A57765Hi | +A*+A*+G*C*T*C*T*A*G*A*T*C*T*T*T*+A* | 12773 | 18 |
| | ATA | | +T*+A | | |
| 768 | TCAAGCTCTAGATCT | A57766Hi | +T*+C*+A*A*G*C*T*C*T*A*G*A*T*C*T*T*+ | 12774 | 19 |
| | TTAT | | T*+A*+T | | |
| 769 | ACTTATCAAGCTCTA | A57767Hi | +A*+C*+T*T*A*T*C*A*A*G*C*T*C*T*A*G*+ | 12779 | 19 |
| | GATC | | A*+T*+C | | |
| 770 | GTTAGACTTATCAAG | A57768Hi | +G*+T*+T*A*G*A*C*T*T*A*T*C*A*A*G*+C* | 12785 | 18 |
| | CTC | | +T*+C | | |
| 771 | TGGCATCAAAAATTA | A57769H | +T*+G*+G*C*A*T*C*A*A*A*A*A*T*T*A*A*+ | 13017 | 19 |
| | ACAT | | C*+A*+T | | |
| 772 | CATGGCATCAAAAAT | A57770H | +C*+A*+T*G*G*C*A*T*C*A*A*A*A*A*T*T*+ | 13019 | 19 |
| | TAAC | | A*+A*+C | | |
| 773 | GTGCATGGCATCAAA | A57771H | +G*+T*+G*C*A*T*G*G*C*A*T*C*A*A*A*A*+ | 13022 | 19 |
| | AATT | | A*+T*+T | | |
| 774 | GTGCATGGCATCAAA | A57772H | +G*+T*+G*C*A*T*G*G*C*A*T*C*A*A*A*+A* | 13023 | 18 |
| | AAT | | +A*+T | | |
| 775 | AGTGCATGGCATCAA | A57773H | +A*+G*+T*G*C*A*T*G*G*C*A*T*C*A*A*A*+ | 13023 | 19 |
| | AAAT | | A*+A*+T | | |
| 776 | AATGAGTGCATGGCA | A57774H | +A*+A*+T*G*A*G*T*G*C*A*T*G*G*C*A*T*+ | 13027 | 19 |
| | TCAA | | C*+A*+A | | |
| 777 | GGAAATGAGTGCATG | A57775H | +G*+G*+A*A*A*T*G*A*G*T*G*C*A*T*G*G* | 13030 | 19 |
| | GCAT | | +C*+A*+T | | |
| 778 | TGGAAATGAGTGCAT | A57776H | +T*+G*+G*A*A*A*T*G*A*G*T*G*C*A*T*G*+ | 13031 | 19 |
| | GGCA | | G*+C*+A | | |
| 779 | TGGAAATGAGTGCAT | A57777H | +T*+G*+G*A*A*A*T*G*A*G*T*G*C*A*T*+G* | 13032 | 18 |
| | GGC | | +G*+C | | |
| 780 | CTGGAAATGAGTGCA | A57778H | +C*+T*+G*G*A*A*A*T*G*A*G*T*G*C*A*T*+ | 13032 | 19 |
| | TGGC | | G*+G*+C | | |
| 781 | TTGGCTGGAAATGAG | A57779H | +T*+T*+G*G*C*T*G*G*A*A*A*T*G*A*G*T*+ | 13036 | 19 |
| | TGCA | | G*+C*+A | | |
| 782 | ATTGGCTGGAAATGA | A57780H | +A*+T*+T*G*G*C*T*G*G*A*A*A*T*G*A*G*+ | 13037 | 19 |
| | GTGC | | T*+G*+C | | |
| 783 | TCATTGGCTGGAAAT | A57781H | +T*+C*+A*T*T*G*G*C*T*G*G*A*A*A*T*G*+ | 13039 | 19 |
| | GAGT | | A*+G*+T | | |
| 784 | ATCATTGGCTGGAAA | A57782H | +A*+T*+C*A*T*T*G*G*C*T*G*G*A*A*A*T*+ | 13040 | 19 |
| | TGAG | | G*+A*+G | | |
| 785 | ATCATTGGCTGGAAA | A57783H | +A*+T*+C*A*T*T*G*G*C*T*G*G*A*A*A*+T* | 13041 | 18 |
| | TGA | | +G*+A | | |
| 786 | CATCATTGGCTGGAA | A57784H | +C*+A*+T*C*A*T*T*G*G*C*T*G*G*A*A*A*+ | 13041 | 19 |
| | ATGA | | T*+G*+A | | |
| 787 | TCATCATTGGCTGGA | A57785H | +T*+C*+A*T*C*A*T*T*G*G*C*T*G*G*A*A*+ | 13042 | 19 |
| | AATG | | A*+T*+G | | |
| 788 | TTCATCATTGGCTGG | A57786H | +T*+T*+C*A*T*C*A*T*T*G*G*C*T*G*G*A*+ | 13043 | 19 |
| | AAAT | | A*+A*+T | | |
| 789 | TTTGCATCCAAGTTT | A57787H | +T*+T*+T*G*C*A*T*C*C*A*A*G*T*T*T*C*+ | 13064 | 19 |
| | CTGG | | T*+G*+G | | |
| 790 | AAGCTTTGCATCCAA | A57788H | +A*+A*+G*C*T*T*T*G*C*A*T*C*C*A*A*G*+ | 13068 | 19 |
| | GTTT | | T*+T*+T | | |
| 791 | GTAAAGCTTTGCATC | A57789H | +G*+T*+A*A*A*G*C*T*T*T*G*C*A*T*C*C*+ | 13071 | 19 |
| | CAAG | | A*+A*+G | | |
| 792 | GTAAAGCTTTGCATC | A57790H | +G*+T*+A*A*A*G*C*T*T*T*G*C*A*T*C*+C* | 13072 | 18 |
| | CAA | | +A*+A | | |
| 793 | TGTAAAGCTTTGCAT | A57791H | +T*+G*+T*A*A*A*G*C*T*T*T*G*C*A*T*C*+ | 13072 | 19 |
| | CCAA | | C*+A*+A | | |
| 794 | TAAACCTTATGCCTT | A57792Hi | +T*+A*+A*A*C*C*T*T*A*T*G*C*C*T*T*+G* | 13349 | 18 |
| | GTT | | +T*+T | | |
| 795 | GTAAACCTTATGCCT | A57793Hi | +G*+T*+A*A*A*C*C*T*T*A*T*G*C*C*T*+T* | 13350 | 18 |
| | TGT | | +G*+T | | |
| 796 | AGTAAACCTTATGCC | A57794Hi | +A*+G*+T*A*A*A*C*C*T*T*A*T*G*C*C*T*+ | 13350 | 19 |
| | TTGT | | T*+G*+T | | |
| 797 | GTAAACCTTATGCCT | A57795Hi | +G*+T*+A*A*A*C*C*T*T*A*T*G*C*C*+T*+T | 13351 | 17 |
| | TG | | *+G | | |
| 798 | AGTAAACCTTATGCC | A57796Hi | +A*+G*+T*A*A*A*C*C*T*T*A*T*G*C*C*+T* | 13351 | 18 |
| | TTG | | +T*+G | | |
| 799 | AAGTAAACCTTATGC | A57797Hi | +A*+A*+G*T*A*A*A*C*C*T*T*A*T*G*C*C*+ | 13351 | 19 |
| | CTTG | | T*+T*+G | | |
| 800 | TAACCTATGCTTGGC | A57798Hi | +T*+A*+A*C*C*T*A*T*G*C*T*T*G*G*C*T*+ | 13414 | 19 |
| | TTCA | | T*+C*+A | | |
| 801 | AACCTATGCTTGGCT | A57799Hi | +A*+A*+C*C*T*A*T*G*C*T*T*G*G*C*T*+T* | 13414 | 18 |
| | TCA | | +C*+A | | |
| 802 | TGTAACCTATGCTTG | A57800Hi | +T*+G*+T*A*A*C*C*T*A*T*G*C*T*T*G*+G* | 13417 | 18 |
| | GCT | | +C*+T | | |
| 803 | GTAACCTATGCTTGG | A57801Hi | +G*+T*+A*A*C*C*T*A*T*G*C*T*T*G*+G*+C | 13417 | 17 |
| | CT | | *+T | | |
| 804 | GACTCAACATCATAA | A57802Hi | +G*+A*+C*T*C*A*A*C*A*T*C*A*T*A*A*+T* | 13438 | 18 |
| | TCC | | +C*+C | | |
| 805 | GACTAAACAAGGACT | A57803Hi | +G*+A*+C*T*A*A*A*C*A*A*G*G*A*C*T*+C* | 13591 | 18 |
| | CCA | | +C*+A | | |
| 806 | TCAATGGAAAGACTA | A57804Hi | +T*+C*+A*A*T*G*G*A*A*A*G*A*C*T*A*+A* | 13601 | 18 |
| | AAC | | +A*+C | | |
| 807 | GTCAATGGAAAGACT | A57805Hi | +G*+T*+C*A*A*T*G*G*A*A*A*G*A*C*T*+A* | 13602 | 18 |
| | AAA | | +A*+A | | |
| 808 | GCTCACTTCACTGCT | A57806Hi | +G*+C*+T*C*A*C*T*T*C*A*C*T*G*C*T*G*+ | 13636 | 19 |
| | GTGA | | T*+G*+A | | |
| 809 | AGGATGCTCACTTCA | A57807Hi | +A*+G*+G*A*T*G*C*T*C*A*C*T*T*C*A*C*+ | 13641 | 19 |
| | CTGC | | T*+G*+C | | |
| 810 | CAGGATGCTCACTTC | A57808Hi | +C*+A*+G*G*A*T*G*C*T*C*A*C*T*T*C*A*+ | 13642 | 19 |
| | ACTG | | C*+T*+G | | |
| 811 | ACTGTACTGTCACAA | A57809Hi | +A*+C*+T*G*T*A*C*T*G*T*C*A*C*A*A*+G* | 13674 | 18 |
| | GCT | | +C*+T | | |
| 812 | GAACTGTACTGTCAC | A57810Hi | +G*+A*+A*C*T*G*T*A*C*T*G*T*C*A*C*A*+ | 13675 | 19 |
| | AAGC | | A*+G*+C | | |
| 813 | TGAACTGTACTGTCA | A57811Hi | +T*+G*+A*A*C*T*G*T*A*C*T*G*T*C*A*+C* | 13677 | 18 |
| | CAA | | +A*+A | | |
| 814 | AATTGAACTGTACTG | A57812Hi | +A*+A*+T*T*G*A*A*C*T*G*T*A*C*T*G*T*+ | 13679 | 19 |
| | TCAC | | C*+A*+C | | |
| 815 | TAATTGAACTGTACT | A57813Hi | +T*+A*+A*T*T*G*A*A*C*T*G*T*A*C*T*G*+ | 13680 | 19 |
| | GTCA | | T*+C*+A | | |
| 816 | CTAATTGAACTGTAC | A57814Hi | +C*+T*+A*A*T*T*G*A*A*C*T*G*T*A*C*+T* | 13682 | 18 |
| | TGT | | +G*+T | | |
| 817 | CTGGTAAACTGATAA | A57815Hi | +C*+T*+G*G*T*A*A*A*C*T*G*A*T*A*A*C*+ | 13786 | 19 |
| | CTGA | | T*+G*+A | | |
| 818 | GCACTCTAAGTGATC | A57816Hi | +G*+C*+A*C*T*C*T*A*A*G*T*G*A*T*C*+A* | 14813 | 18 |
| | AAC | | +A*+C | | |
| 819 | TAGTTTATCTAGCTT | A57817Hi | +T*+A*+G*T*T*T*A*T*C*T*A*G*C*T*T*+G* | 14861 | 18 |
| | GAG | | +A*+G | | |
| 820 | GATCAAGCCAGTAAG | A57818Hi | +G*+A*+T*C*A*A*G*C*C*A*G*T*A*A*G*+T* | 14931 | 18 |
| | TTC | | +T*+C | | |
| 821 | ATTTGATCAAGCCAG | A57819Hi | +A*+T*+T*T*G*A*T*C*A*A*G*C*C*A*G*T*+ | 14934 | 19 |
| | TAAG | | A*+A*+G | | |
| 822 | CATTTGATCAAGCCA | A57820Hi | +C*+A*+T*T*T*G*A*T*C*A*A*G*C*C*A*G*+ | 14935 | 19 |
| | GTAA | | T*+A*+A | | |
| 823 | AGTCATTTGATCAAG | A57821Hi | +A*+G*+T*C*A*T*T*T*G*A*T*C*A*A*G*+C* | 14939 | 18 |
| | CCA | | +C*+A | | |
| 824 | AAGTCATTTGATCAA | A57822Hi | +A*+A*+G*T*C*A*T*T*T*G*A*T*C*A*A*G*+ | 14939 | 19 |
| | GCCA | | C*+C*+A | | |
| 825 | AAAGTCATTTGATCA | A57823Hi | +A*+A*+A*G*T*C*A*T*T*T*G*A*T*C*A*A*+ | 14940 | 19 |
| | AGCC | | G*+C*+C | | |
| 826 | AATTAAAGTCATTTG | A57824Hi | +A*+A*+T*T*A*A*A*G*T*C*A*T*T*T*G*A*+ | 14944 | 19 |
| | ATCA | | T*+C*+A | | |
| 827 | CAATTAAAGTCATTT | A57825Hi | +C*+A*+A*T*T*A*A*A*G*T*C*A*T*T*T*G*+ | 14945 | 19 |
| | GATC | | A*+T*+C | | |
| 828 | GCAAATGGCAAGTAA | A57826Hi | +G*+C*+A*A*A*T*G*G*C*A*A*G*T*A*A*T*+ | 15029 | 19 |
| | TCAG | | C*+A*+G | | |
| 829 | GAGCAAATGGCAAGT | A57827Hi | +G*+A*+G*C*A*A*A*T*G*G*C*A*A*G*T*A*+ | 15031 | 19 |
| | AATC | | A*+T*+C | | |
| 830 | ACTTGAGCAAATGGC | A57828Hi | +A*+C*+T*T*G*A*G*C*A*A*A*T*G*G*C*A*+ | 15035 | 19 |
| | AAGT | | A*+G*+T | | |
| 831 | CTTGAGCAAATGGCA | A57829Hi | +C*+T*+T*G*A*G*C*A*A*A*T*G*G*C*A*+A* | 15035 | 18 |
| | AGT | | +G*+T | | |
| 832 | AACTTGAGCAAATGG | A57830Hi | +A*+A*+C*T*T*G*A*G*C*A*A*A*T*G*G*C*+ | 15036 | 19 |
| | CAAG | | A*+A*+G | | |
| 833 | TGTCAACTTGAGCAA | A57831Hi | +T*+G*+T*C*A*A*C*T*T*G*A*G*C*A*A*A*+ | 15040 | 19 |
| | ATGG | | T*+G*+G | | |
| 834 | GTCAACTTGAGCAAA | A57832Hi | +G*+T*+C*A*A*C*T*T*G*A*G*C*A*A*A*+T* | 15040 | 18 |
| | TGG | | +G*+G | | |
| 835 | AGTTGTCAACTTGAG | A57833Hi | +A*+G*+T*T*G*T*C*A*A*C*T*T*G*A*G*C*+ | 15043 | 19 |
| | CAAA | | A*+A*+A | | |
| 836 | AGAGTTGTCAACTTG | A57834Hi | +A*+G*+A*G*T*T*G*T*C*A*A*C*T*T*G*A*+ | 15045 | 19 |
| | AGCA | | G*+C*+A | | |
| 837 | TTCAGTTCAAGAGTT | A57835Hi | +T*+T*+C*A*G*T*T*C*A*A*G*A*G*T*T*G*+ | 15054 | 19 |
| | GTCA | | T*+C*+A | | |
| 838 | GTTTCAGTTCAAGAG | A57836Hi | +G*+T*+T*T*C*A*G*T*T*C*A*A*G*A*G*T*+ | 15056 | 19 |
| | TTGT | | T*+G*+T | | |
| 839 | CGGCTAAATGTTTCA | A57837Hi | +C*+G*+G*C*T*A*A*A*T*G*T*T*T*C*A*+G* | 15066 | 18 |
| | GTT | | +T*+T | | |
| 840 | TCGGCTAAATGTTTC | A57838Hi | +T*+C*+G*G*C*T*A*A*A*T*G*T*T*T*C*A*+ | 15066 | 19 |
| | AGTT | | G*+T*+T | | |
| 841 | CGGCTAAATGTTTCA | A57839Hi | +C*+G*+G*C*T*A*A*A*T*G*T*T*T*C*+A*+G | 15067 | 17 |
| | GT | | *+T | | |
| 842 | CTCGGCTAAATGTTT | A57840Hi | +C*+T*+C*G*G*C*T*A*A*A*T*G*T*T*T*C*+ | 15067 | 19 |
| | CAGT | | A*+G*+T | | |
| 843 | CTCGGCTAAATGTTT | A57841Hi | +C*+T*+C*G*G*C*T*A*A*A*T*G*T*T*T*+C* | 15068 | 18 |
| | CAG | | +A*+G | | |
| 844 | TCGGCTAAATGTTTC | A57842Hi | +T*+C*+G*G*C*T*A*A*A*T*G*T*T*T*+C*+A | 15068 | 17 |
| | AG | | *+G | | |
| 845 | GCATTAATGGTAGGC | A57843Hi | +G*+C*+A*T*T*A*A*T*G*G*T*A*G*G*C*+T* | 15096 | 18 |
| | TGC | | +G*+C | | |
| 846 | AGGCATTAATGGTAG | A57844Hi | +A*+G*+G*C*A*T*T*A*A*T*G*G*T*A*+G*+ | 15099 | 17 |
| | GC | | G*+C | | |
| 847 | CAGTGACTAATAAAG | A57845Hi | +C*+A*+G*T*G*A*C*T*A*A*T*A*A*A*G*A*+ | 15153 | 19 |
| | ATGG | | T*+G*+G | | |
| 848 | GACAGTGACTAATAA | A57846Hi | +G*+A*+C*A*G*T*G*A*C*T*A*A*T*A*A*+A* | 15156 | 18 |
| | AGA | | +G*+A | | |
| 849 | ATCTTTGTTCTCAACA | A57847H | +A*+T*+C*T*T*T*G*T*T*C*T*C*A*A*C*A*+ | 15509 | 19 |
| | AAT | | A*+A*+T | | |
| 850 | AATCTTTGTTCTCAAC | A57848H | +A*+A*+T*C*T*T*T*G*T*T*C*T*C*A*A*C*+ | 15510 | 19 |
| | AAA | | A*+A*+A | | |
| 851 | AATCTTTGTTCTCAAC | A57849H | +A*+A*+T*C*T*T*T*G*T*T*C*T*C*A*A*+C* | 15511 | 18 |
| | AA | | +A*+A | | |
| 852 | TAATCTTTGTTCTCAA | A57850H | +T*+A*+A*T*C*T*T*T*G*T*T*C*T*C*A*A*+ | 15511 | 19 |
| | CAA | | C*+A*+A | | |
| 853 | ATAATCTTTGTTCTCA | A57851H | +A*+T*+A*A*T*C*T*T*T*G*T*T*C*T*C*A*+ | 15512 | 19 |
| | ACA | | A*+C*+A | | |
| 854 | TATAATCTTTGTTCTC | A57852H | +T*+A*+T*A*A*T*C*T*T*T*G*T*T*C*T*C*+ | 15513 | 19 |
| | AAC | | A*+A*+C | | |
| 855 | CATATAATCTTTGTTC | A57853H | +C*+A*+T*A*T*A*A*T*C*T*T*T*G*T*T*C*+ | 15515 | 19 |
| | TCA | | T*+C*+A | | |
| 856 | TTAACAATATCATATA | A57854H | +T*+T*+A*A*C*A*A*T*A*T*C*A*T*A*T*A*+ | 15525 | 19 |
| | ATC | | A*+T*+C | | |
| 857 | GCACTTAACAATATC | A57855H | +G*+C*+A*C*T*T*A*A*C*A*A*T*A*T*C*A*+ | 15529 | 19 |
| | ATAT | | T*+A*+T | | |
| 858 | CGCACTTAACAATAT | A57856H | +C*+G*+C*A*C*T*T*A*A*C*A*A*T*A*T*C*+ | 15530 | 19 |
| | CATA | | A*+T*+A | | |
| 859 | CGCACTTAACAATAT | A57857H | +C*+G*+C*A*C*T*T*A*A*C*A*A*T*A*T*+C* | 15531 | 18 |
| | CAT | | +A*+T | | |
| 860 | ACGCACTTAACAATA | A57858H | +A*+C*+G*C*A*C*T*T*A*A*C*A*A*T*A*T*+ | 15531 | 19 |
| | TCAT | | C*+A*+T | | |
| 861 | TTAACGCACTTAACA | A57859H | +T*+T*+A*A*C*G*C*A*C*T*T*A*A*C*A*A*+ | 15534 | 19 |
| | ATAT | | T*+A*+T | | |
| 862 | TTAACGCACTTAACA | A57860H | +T*+T*+A*A*C*G*C*A*C*T*T*A*A*C*A*+A* | 15535 | 18 |
| | ATA | | +T*+A | | |
| 863 | GTTAACGCACTTAAC | A57861H | +G*+T*+T*A*A*C*G*C*A*C*T*T*A*A*C*A*+ | 15535 | 19 |
| | AATA | | A*+T*+A | | |
| 864 | TTAACGCACTTAACA | A57862H | +T*+T*+A*A*C*G*C*A*C*T*T*A*A*C*+A*+A | 15536 | 17 |
| | AT | | *+T | | |
| 865 | GTTAACGCACTTAAC | A57863H | +G*+T*+T*A*A*C*G*C*A*C*T*T*A*A*C*+A* | 15536 | 18 |
| | AAT | | +A*+T | | |
| 866 | TGTTAACGCACTTAA | A57864H | +T*+G*+T*T*A*A*C*G*C*A*C*T*T*A*A*C*+ | 15536 | 19 |
| | CAAT | | A*+A*+T | | |
| 867 | TTTGTTAACGCACTT | A57865H | +T*+T*+T*G*T*T*A*A*C*G*C*A*C*T*T*A*+ | 15538 | 19 |
| | AACA | | A*+C*+A | | |
| 868 | TTTGTTAACGCACTT | A57866H | +T*+T*+T*G*T*T*A*A*C*G*C*A*C*T*T*+A* | 15539 | 18 |
| | AAC | | +A*+C | | |
| 869 | ATTTGTTAACGCACT | A57867H | +A*+T*+T*T*G*T*T*A*A*C*G*C*A*C*T*T*+ | 15539 | 19 |
| | TAAC | | A*+A*+C | | |
| 870 | AAATTTGTTAACGCA | A57868H | +A*+A*+A*T*T*T*G*T*T*A*A*C*G*C*A*C*+ | 15541 | 19 |
| | CTTA | | T*+T*+A | | |
| 871 | TTAAAATTTGTTAAC | A57869H | +T*+T*+A*A*A*A*T*T*T*G*T*T*A*A*C*G*+ | 15544 | 19 |
| | GCAC | | C*+A*+C | | |
| 872 | ATGAAGCACAATTTC | A57870H | +A*+T*+G*A*A*G*C*A*C*A*A*T*T*T*C*C*+ | 15569 | 19 |
| | CTCT | | T*+C*+T | | |
| 873 | ACATGAAGCACAATT | A57871H | +A*+C*+A*T*G*A*A*G*C*A*C*A*A*T*T*T*+ | 15571 | 19 |
| | TCCT | | C*+C*+T | | |
| 874 | AGGGAATGTAAACAA | A57872H | +A*+G*+G*G*A*A*T*G*T*A*A*A*C*A*A*T*+ | 15592 | 19 |
| | TGCA | | G*+C*+A | | |
| 875 | GCTAGGGAATGTAAA | A57873H | +G*+C*+T*A*G*G*G*A*A*T*G*T*A*A*A*C*+ | 15595 | 19 |
| | CAAT | | A*+A*+T | | |
| 876 | CCACAGCCAACTTCA | A57874Hi | +C*+C*+A*C*A*G*C*C*A*A*C*T*T*C*A*G*+ | 15781 | 19 |
| | GTAT | | T*+A*+T | | |
| 877 | TTGTACTACAAGCTC | A57875Hi | +T*+T*+G*T*A*C*T*A*C*A*A*G*C*T*C*T*+ | 15851 | 19 |
| | TGAA | | G*+A*+A | | |
| 878 | TTGTACTACAAGCTC | A57876Hi | +T*+T*+G*T*A*C*T*A*C*A*A*G*C*T*C*+T* | 15852 | 18 |
| | TGA | | +G*+A | | |
| 879 | GCAGTCTGCTCTCTC | A57877Hi | +G*+C*+A*G*T*C*T*G*C*T*C*T*C*T*C*T*+ | 16085 | 19 |
| | TTGC | | T*+G*+C | | |
| 880 | GGCAGTCTGCTCTCT | A57878Hi | +G*+G*+C*A*G*T*C*T*G*C*T*C*T*C*T*C*+ | 16086 | 19 |
| | CTTG | | T*+T*+G | | |
| 881 | GCAGTCTGCTCTCTC | A57879Hi | +G*+C*+A*G*T*C*T*G*C*T*C*T*C*T*C*+T* | 16086 | 18 |
| | TTG | | +T*+G | | |
| 882 | AGGCAGTCTGCTCTC | A57880Hi | +A*+G*+G*C*A*G*T*C*T*G*C*T*C*T*C*T*+ | 16087 | 19 |
| | TCTT | | C*+T*+T | | |
| 883 | TTGAAGGCAGTCTGC | A57881Hi | +T*+T*+G*A*A*G*G*C*A*G*T*C*T*G*C*T*+ | 16091 | 19 |
| | TCTC | | C*+T*+C | | |
| 884 | TTCTTGAAGGCAGTC | A57882Hi | +T*+T*+C*T*T*G*A*A*G*G*C*A*G*T*C*T*+ | 16094 | 19 |
| | TGCT | | G*+C*+T | | |
| 885 | CTTCTTGAAGGCAGT | A57883Hi | +C*+T*+T*C*T*T*G*A*A*G*G*C*A*G*T*C*+ | 16095 | 19 |
| | CTGC | | T*+G*+C | | |
| 886 | TTCTTGAAGGCAGTC | A57884Hi | +T*+T*+C*T*T*G*A*A*G*G*C*A*G*T*C*+T* | 16095 | 18 |
| | TGC | | +G*+C | | |
| 887 | GTCTTCTTGAAGGCA | A57885Hi | +G*+T*+C*T*T*C*T*T*G*A*A*G*G*C*A*G*+ | 16097 | 19 |
| | GTCT | | T*+C*+T | | |
| 888 | TGTCTTCTTGAAGGC | A57886Hi | +T*+G*+T*C*T*T*C*T*T*G*A*A*G*G*C*A*+ | 16098 | 19 |
| | AGTC | | G*+T*+C | | |
| 889 | GTCTTCTTGAAGGCA | A57887Hi | +G*+T*+C*T*T*C*T*T*G*A*A*G*G*C*A*+G* | 16098 | 18 |
| | GTC | | +T*+C | | |
| 890 | TTGTCTTCTTGAAGG | A57888Hi | +T*+T*+G*T*C*T*T*C*T*T*G*A*A*G*G*C*+ | 16099 | 19 |
| | CAGT | | A*+G*+T | | |
| 891 | GCCTCTGTCAATGAA | A57889Hi | +G*+C*+C*T*C*T*G*T*C*A*A*T*G*A*A*A*+ | 16211 | 19 |
| | ACAC | | C*+A*+C | | |
| 892 | TGCCTCTGTCAATGA | A57890Hi | +T*+G*+C*C*T*C*T*G*T*C*A*A*T*G*A*A*+ | 16212 | 19 |
| | AACA | | A*+C*+A | | |
| 893 | CCACTCCATGTCTCT | A57891Hi | +C*+C*+A*C*T*C*C*A*T*G*T*C*T*C*T*G*+ | 16261 | 19 |
| | GGCC | | G*+C*+C | | |
| 894 | TCCACTCCATGTCTC | A57892Hi | +T*+C*+C*A*C*T*C*C*A*T*G*T*C*T*C*T*+ | 16262 | 19 |
| | TGGC | | G*+G*+C | | |
| 895 | TTCATGCTCTCCACT | A57893Hi | +T*+T*+C*A*T*G*C*T*C*T*C*C*A*C*T*C*+ | 16271 | 19 |
| | CCAT | | C*+A*+T | | |
| 896 | GTATATCCTGTAGAA | A57894Hi | +G*+T*+A*T*A*T*C*C*T*G*T*A*G*A*A*+A* | 16548 | 18 |
| | ACC | | +C*+C | | |
| 897 | TAAATGCATATGTAT | A57895Hi | +T*+A*+A*A*T*G*C*A*T*A*T*G*T*A*T*A*+ | 16558 | 19 |
| | ATCC | | T*+C*+C | | |
| 898 | CACATGTGTGAGGAT | A57896Hi | +C*+A*+C*A*T*G*T*G*T*G*A*G*G*A*T*G*+ | 16588 | 19 |
| | GAAA | | A*+A*+A | | |
| 899 | AATAATCCTGTCAGT | A57897Hi | +A*+A*+T*A*A*T*C*C*T*G*T*C*A*G*T*G*+ | 16661 | 19 |
| | GATT | | A*+T*+T | | |
| 900 | TAATAATCCTGTCAG | A57898Hi | +T*+A*+A*T*A*A*T*C*C*T*G*T*C*A*G*+T* | 16663 | 18 |
| | TGA | | +G*+A | | |
| 901 | GCAATTACATATAGT | A57899Hi | +G*+C*+A*A*T*T*A*C*A*T*A*T*A*G*T*A*+ | 16707 | 19 |
| | ATAC | | T*+A*+C | | |
| 902 | TGACACAGTGATTTC | A57900Hi | +T*+G*+A*C*A*C*A*G*T*G*A*T*T*T*C*C*+ | 16741 | 19 |
| | CATA | | A*+T*+A | | |
| 903 | TAATTTCCTGTGGAT | A57901Hi | +T*+A*+A*T*T*T*C*C*T*G*T*G*G*A*T*A*+ | 16857 | 19 |
| | AAGA | | A*+G*+A | | |
| 904 | TCTCTATTATTGTTCA | A57902Hi | +T*+C*+T*C*T*A*T*T*A*T*T*G*T*T*C*A*+ | 16895 | 19 |
| | GCC | | G*+C*+C | | |
| 905 | GGCCTTTCTCTATTAT | A57903Hi | +G*+G*+C*C*T*T*T*C*T*C*T*A*T*T*A*T*+ | 16901 | 19 |
| | TGT | | T*+G*+T | | |
| 906 | TGGCCTTTCTCTATTA | A57904Hi | +T*+G*+G*C*C*T*T*T*C*T*C*T*A*T*T*A*+ | 16902 | 19 |
| | TTG | | T*+T*+G | | |
| 907 | CAATAGACTTAAAGA | A57905Hi | +C*+A*+A*T*A*G*A*C*T*T*A*A*A*G*A*+C* | 17181 | 18 |
| | CTG | | +T*+G | | |
| 908 | ACAATAGACTTAAAG | A57906Hi | +A*+C*+A*A*T*A*G*A*C*T*T*A*A*A*G*A*+ | 17181 | 19 |
| | ACTG | | C*+T*+G | | |
| 909 | GCATCAAATGTTAAC | A57907Hi | +G*+C*+A*T*C*A*A*A*T*G*T*T*A*A*C*T*+ | 17355 | 19 |
| | TGTT | | G*+T*+T | | |
| 910 | AGCTTCCTGAAGGCT | A57908Hi | +A*+G*+C*T*T*C*C*T*G*A*A*G*G*C*T*C*+ | 17548 | 19 |
| | CCAA | | C*+A*+A | | |
| 911 | TCTTAACGAGGCTCA | A57909Hi | +T*+C*+T*T*A*A*C*G*A*G*G*C*T*C*A*+A* | 17716 | 18 |
| | ACC | | +C*+C | | |
| 912 | TTCTTAACGAGGCTC | A57910Hi | +T*+T*+C*T*T*A*A*C*G*A*G*G*C*T*C*A*+ | 17716 | 19 |
| | AACC | | A*+C*+C | | |
| 913 | TTCTTAACGAGGCTC | A57911Hi | +T*+T*+C*T*T*A*A*C*G*A*G*G*C*T*C*+A* | 17717 | 18 |
| | AAC | | +A*+C | | |
| 914 | GCTTAAAGCCTTAAG | A57912Hi | +G*+C*+T*T*A*A*A*G*C*C*T*T*A*A*G*+A* | 18299 | 18 |
| | ATC | | +T*+C | | |
| 915 | GGCTTAAAGCCTTAA | A57913Hi | +G*+G*+C*T*T*A*A*A*G*C*C*T*T*A*A*+G* | 18300 | 18 |
| | GAT | | +A*+T | | |
| 916 | TGGCTTAAAGCCTTA | A57914Hi | +T*+G*+G*C*T*T*A*A*A*G*C*C*T*T*A*+A* | 18301 | 18 |
| | AGA | | +G*+A | | |
| 917 | CTGTTTCTTGGCTTA | A57915Hi | +C*+T*+G*T*T*T*C*T*T*G*G*C*T*T*A*A*+ | 18308 | 19 |
| | AAGC | | A*+G*+C | | |
| 918 | CCAAATTTAAACATC | A57916Hi | +C*+C*+A*A*A*T*T*T*A*A*A*C*A*T*C*A*+ | 18421 | 19 |
| | ATGT | | T*+G*+T | | |
| 919 | TCCAAATTTAAACAT | A57917Hi | +T*+C*+C*A*A*A*T*T*T*A*A*A*C*A*T*C*+ | 18422 | 19 |
| | CATG | | A*+T*+G | | |
| 920 | AGATGGCAGCATAAT | A57918Hi | +A*+G*+A*T*G*G*C*A*G*C*A*T*A*A*T*+G* | 18447 | 18 |
| | GAC | | +A*+C | | |
| 921 | ATAGATGGCAGCATA | A57919Hi | +A*+T*+A*G*A*T*G*G*C*A*G*C*A*T*A*+A* | 18449 | 18 |
| | ATG | | +T*+G | | |
| 922 | TAAATAGATGGCAGC | A57920Hi | +T*+A*+A*A*T*A*G*A*T*G*G*C*A*G*C*A*+ | 18451 | 19 |
| | ATAA | | T*+A*+A | | |
| 923 | ATCATAATTATCAATA | A57921Hi | +A*+T*+C*A*T*A*A*T*T*A*T*C*A*A*T*A*+ | 18521 | 19 |
| | AGG | | A*+G*+G | | |
| 924 | TCATGAGGACTTCCA | A57922H | +T*+C*+A*T*G*A*G*G*A*C*T*T*C*C*A*C*+ | 18559 | 19 |
| | CATT | | A*+T*+T | | |
| 925 | CTCATGAGGACTTCC | A57923H | +C*+T*+C*A*T*G*A*G*G*A*C*T*T*C*C*A*+ | 18560 | 19 |
| | ACAT | | C*+A*+T | | |
| 926 | CTCATGAGGACTTCC | A57924H | +C*+T*+C*A*T*G*A*G*G*A*C*T*T*C*C*+A* | 18561 | 18 |
| | ACA | | +C*+A | | |
| 927 | ACTCATGAGGACTTC | A57925H | +A*+C*+T*C*A*T*G*A*G*G*A*C*T*T*C*C*+ | 18561 | 19 |
| | CACA | | A*+C*+A | | |
| 928 | GCCACTCATGAGGAC | A57926H | +G*+C*+C*A*C*T*C*A*T*G*A*G*G*A*C*T*+ | 18564 | 19 |
| | TTCC | | T*+C*+C | | |
| 929 | ATTGCCACTCATGAG | A57927H | +A*+T*+T*G*C*C*A*C*T*C*A*T*G*A*G*G*+ | 18567 | 19 |
| | GACT | | A*+C*+T | | |
| 930 | CATTGCCACTCATGA | A57928H | +C*+A*+T*T*G*C*C*A*C*T*C*A*T*G*A*G*+ | 18568 | 19 |
| | GGAC | | G*+A*+C | | |
| 931 | TGACATTGCCACTCA | A57929H | +T*+G*+A*C*A*T*T*G*C*C*A*C*T*C*A*T*+ | 18571 | 19 |
| | TGAG | | G*+A*+G | | |
| 932 | CTGACATTGCCACTC | A57930H | +C*+T*+G*A*C*A*T*T*G*C*C*A*C*T*C*A*+ | 18572 | 19 |
| | ATGA | | T*+G*+A | | |
| 933 | CCTGACATTGCCACT | A57931H | +C*+C*+T*G*A*C*A*T*T*G*C*C*A*C*T*C*+ | 18573 | 19 |
| | CATG | | A*+T*+G | | |
| 934 | ACCTGACATTGCCAC | A57932H | +A*+C*+C*T*G*A*C*A*T*T*G*C*C*A*C*T*+ | 18574 | 19 |
| | TCAT | | C*+A*+T | | |
| 935 | ACACCTGACATTGCC | A57933H | +A*+C*+A*C*C*T*G*A*C*A*T*T*G*C*C*A*+ | 18576 | 19 |
| | ACTC | | C*+T*+C | | |
| 936 | AACACCTGACATTGC | A57934H | +A*+A*+C*A*C*C*T*G*A*C*A*T*T*G*C*C*+ | 18577 | 19 |
| | CACT | | A*+C*+T | | |
| 937 | TAACACCTGACATTG | A57935H | +T*+A*+A*C*A*C*C*T*G*A*C*A*T*T*G*C*+ | 18578 | 19 |
| | CCAC | | C*+A*+C | | |
| 938 | ATAACACCTGACATT | A57936H | +A*+T*+A*A*C*A*C*C*T*G*A*C*A*T*T*G*+ | 18579 | 19 |
| | GCCA | | C*+C*+A | | |
| 939 | ATTATAACACCTGAC | A57937H | +A*+T*+T*A*T*A*A*C*A*C*C*T*G*A*C*A*+ | 18582 | 19 |
| | ATTG | | T*+T*+G | | |
| 940 | CACAATATTATAACA | A57938H | +C*+A*+C*A*A*T*A*T*T*A*T*A*A*C*A*C*+ | 18588 | 19 |
| | CCTG | | C*+T*+G | | |
| 941 | TCCACCACAATATTA | A57939H | +T*+C*+C*A*C*C*A*C*A*A*T*A*T*T*A*T*+ | 18593 | 19 |
| | TAAC | | A*+A*+C | | |
| 942 | CTTCCACCACAATAT | A57940H | +C*+T*+T*C*C*A*C*C*A*C*A*A*T*A*T*T*+ | 18595 | 19 |
| | TATA | | A*+T*+A | | |
| 943 | CTTCCACCACAATAT | A57941H | +C*+T*+T*C*C*A*C*C*A*C*A*A*T*A*T*+T* | 18596 | 18 |
| | TAT | | +A*+T | | |
| 944 | GCTTCCACCACAATA | A57942H | +G*+C*+T*T*C*C*A*C*C*A*C*A*A*T*A*T*+ | 18596 | 19 |
| | TTAT | | T*+A*+T | | |
| 945 | GCTTCCACCACAATA | A57943H | +G*+C*+T*T*C*C*A*C*C*A*C*A*A*T*A*+T* | 18597 | 18 |
| | TTA | | +T*+A | | |
| 946 | AGCTTCCACCACAAT | A57944H | +A*+G*+C*T*T*C*C*A*C*C*A*C*A*A*T*A*+ | 18597 | 19 |
| | ATTA | | T*+T*+A | | |
| 947 | CATAGCTTCCACCAC | A57945H | +C*+A*+T*A*G*C*T*T*C*C*A*C*C*A*C*A*+ | 18600 | 19 |
| | AATA | | A*+T*+A | | |
| 948 | TCATAGCTTCCACCA | A57946H | +T*+C*+A*T*A*G*C*T*T*C*C*A*C*C*A*C*+ | 18601 | 19 |
| | CAAT | | A*+A*+T | | |
| 949 | TTCATAGCTTCCACC | A57947H | +T*+T*+C*A*T*A*G*C*T*T*C*C*A*C*C*A*+ | 18602 | 19 |
| | ACAA | | C*+A*+A | | |
| 950 | AATGCTTTCATAGCT | A57948H | +A*+A*+T*G*C*T*T*T*C*A*T*A*G*C*T*T*+ | 18608 | 19 |
| | TCCA | | C*+C*+A | | |
| 951 | GAATGCTTTCATAGC | A57949H | +G*+A*+A*T*G*C*T*T*T*C*A*T*A*G*C*T*+ | 18609 | 19 |
| | TTCC | | T*+C*+C | | |
| 952 | GGAATGCTTTCATAG | A57950H | +G*+G*+A*A*T*G*C*T*T*T*C*A*T*A*G*C*+ | 18610 | 19 |
| | CTTC | | T*+T*+C | | |
| 953 | AGGGAATGCTTTCAT | A57951H | +A*+G*+G*G*A*A*T*G*C*T*T*T*C*A*T*A*+ | 18612 | 19 |
| | AGCT | | G*+C*+T | | |
| 954 | GCAAACAGCAACTCA | A57952H | +G*+C*+A*A*A*C*A*G*C*A*A*C*T*C*A*C*+ | 18649 | 19 |
| | CTTC | | T*+T*+C | | |
| 955 | GCAAACAGCAACTCA | A57953H | +G*+C*+A*A*A*C*A*G*C*A*A*C*T*C*A*+C* | 18650 | 18 |
| | CTT | | +T*+T | | |
| 956 | AGCAAACAGCAACTC | A57954H | +A*+G*+C*A*A*A*C*A*G*C*A*A*C*T*C*A*+ | 18650 | 19 |
| | ACTT | | C*+T*+T | | |
| 957 | GGAGCAAACAGCAAC | A57955H | +G*+G*+A*G*C*A*A*A*C*A*G*C*A*A*C*T*+ | 18652 | 19 |
| | TCAC | | C*+A*+C | | |
| 958 | CTATGGAGCAAACAG | A57956H | +C*+T*+A*T*G*G*A*G*C*A*A*A*C*A*G*C*+ | 18656 | 19 |
| | CAAC | | A*+A*+C | | |
| 959 | TAAGCCTATGGAGCA | A57957H | +T*+A*+A*G*C*C*T*A*T*G*G*A*G*C*A*A*+ | 18661 | 19 |
| | AACA | | A*+C*+A | | |
| 960 | TAAGCCTATGGAGCA | A57958H | +T*+A*+A*G*C*C*T*A*T*G*G*A*G*C*A*+A* | 18662 | 18 |
| | AAC | | +A*+C | | |
| 961 | GTAAGCCTATGGAGC | A57959H | +G*+T*+A*A*G*C*C*T*A*T*G*G*A*G*C*A*+ | 18662 | 19 |
| | AAAC | | A*+A*+C | | |
| 962 | TAATGTAAGCCTATG | A57960H | +T*+A*+A*T*G*T*A*A*G*C*C*T*A*T*G*G*+ | 18666 | 19 |
| | GAGC | | A*+G*+C | | |
| 963 | CTAATGTAAGCCTAT | A57961H | +C*+T*+A*A*T*G*T*A*A*G*C*C*T*A*T*G*+ | 18667 | 19 |
| | GGAG | | G*+A*+G | | |
| 964 | TATGTCAATATGCTA | A57962Hi | +T*+A*+T*G*T*C*A*A*T*A*T*G*C*T*A*C*+ | 18769 | 19 |
| | CTTA | | T*+T*+A | | |
| 965 | AGTATGTCAATATGC | A57963Hi | +A*+G*+T*A*T*G*T*C*A*A*T*A*T*G*C*T*+ | 18771 | 19 |
| | TACT | | A*+C*+T | | |
| 966 | TGTTAACCTCTATAG | A57964Hi | +T*+G*+T*T*A*A*C*C*T*C*T*A*T*A*G*A*+ | 19145 | 19 |
| | AGAA | | G*+A*+A | | |
| 967 | GTTAACCTCTATAGA | A57965Hi | +G*+T*+T*A*A*C*C*T*C*T*A*T*A*G*A*+G* | 19145 | 18 |
| | GAA | | +A*+A | | |
| 968 | TGTTAACCTCTATAG | A57966Hi | +T*+G*+T*T*A*A*C*C*T*C*T*A*T*A*G*+A* | 19146 | 18 |
| | AGA | | +G*+A | | |
| 969 | GTGTTAACCTCTATA | A57967Hi | +G*+T*+G*T*T*A*A*C*C*T*C*T*A*T*A*+G* | 19147 | 18 |
| | GAG | | +A*+G | | |
| 970 | GATAGTGTTAACCTC | A57968Hi | +G*+A*+T*A*G*T*G*T*T*A*A*C*C*T*C*+T* | 19151 | 18 |
| | TAT | | +A*+T | | |
| 971 | TGGATAGTGTTAACC | A57969Hi | +T*+G*+G*A*T*A*G*T*G*T*T*A*A*C*C*T*+ | 19152 | 19 |
| | TCTA | | C*+T*+A | | |
| 972 | ACAGATAGTAGTAGC | A57970Hi | +A*+C*+A*G*A*T*A*G*T*A*G*T*A*G*C*A*+ | 19442 | 19 |
| | ATAA | | T*+A*+A | | |
| 973 | TCACAAAGGAACTGA | A57971Hi | +T*+C*+A*C*A*A*A*G*G*A*A*C*T*G*A*T*+ | 20096 | 19 |
| | TTAT | | T*+A*+T | | |
| 974 | AACTAGCTCTCCACA | A57972Hi | +A*+A*+C*T*A*G*C*T*C*T*C*C*A*C*A*+C* | 20266 | 18 |
| | CTG | | +T*+G | | |
| 975 | ATGGTAACTAGCTCT | A57973Hi | +A*+T*+G*G*T*A*A*C*T*A*G*C*T*C*T*+C* | 20271 | 18 |
| | CCA | | +C*+A | | |
| 976 | TGATGGTAACTAGCT | A57974Hi | +T*+G*+A*T*G*G*T*A*A*C*T*A*G*C*T*+C* | 20273 | 18 |
| | CTC | | +T*+C | | |
| 977 | GTGAACCTCTAATAA | A57975Hi | +G*+T*+G*A*A*C*C*T*C*T*A*A*T*A*A*A*+ | 20296 | 19 |
| | ATTG | | T*+T*+G | | |
| 978 | GAAACAGGTGAACCT | A57976Hi | +G*+A*+A*A*C*A*G*G*T*G*A*A*C*C*T*C*+ | 20303 | 19 |
| | CTAA | | T*+A*+A | | |
| 979 | AGACAAATCAGATTA | A57977Hi | +A*+G*+A*C*A*A*A*T*C*A*G*A*T*T*A*A*+ | 20386 | 19 |
| | ATGC | | T*+G*+C | | |
| 980 | CAGACAAATCAGATT | A57978Hi | +C*+A*+G*A*C*A*A*A*T*C*A*G*A*T*T*A*+ | 20387 | 19 |
| | AATG | | A*+T*+G | | |
| 981 | GATTCAGACAAATCA | A57979Hi | +G*+A*+T*T*C*A*G*A*C*A*A*A*T*C*A*G*+ | 20391 | 19 |
| | GATT | | A*+T*+T | | |
| 982 | AAGGCAAGCTGTGCC | A57980Hi | +A*+A*+G*G*C*A*A*G*C*T*G*T*G*C*C*T*+ | 20509 | 19 |
| | TAAA | | A*+A*+A | | |
| 983 | AAAGGCAAGCTGTGC | A57981Hi | +A*+A*+A*G*G*C*A*A*G*C*T*G*T*G*C*C*+ | 20510 | 19 |
| | CTAA | | T*+A*+A | | |
| 984 | AAGGCAAGCTGTGCC | A57982Hi | +A*+A*+G*G*C*A*A*G*C*T*G*T*G*C*C*+T* | 20510 | 18 |
| | TAA | | +A*+A | | |
| 985 | AGAGTTTAGAAAGGT | A57983Hi | +A*+G*+A*G*T*T*T*A*G*A*A*A*G*G*T*A*+ | 20812 | 19 |
| | AGGT | | G*+G*+T | | |
| 986 | GAACTCAGACTGTCA | A57984Hi | +G*+A*+A*C*T*C*A*G*A*C*T*G*T*C*A*T*+ | 20899 | 19 |
| | TTAT | | T*+A*+T | | |
| 987 | GAACTCAGACTGTCA | A57985Hi | +G*+A*+A*C*T*C*A*G*A*C*T*G*T*C*A*+T* | 20900 | 18 |
| | TTA | | +T*+A | | |
| 988 | CTGTAGATGTATTTC | A57986Hi | +C*+T*+G*T*A*G*A*T*G*T*A*T*T*T*C*T*+ | 21288 | 19 |
| | TTCA | | T*+C*+A | | |
| 989 | AAACCTGTAGATGTA | A57987Hi | +A*+A*+A*C*C*T*G*T*A*G*A*T*G*T*A*T*+ | 21292 | 19 |
| | TTTC | | T*+T*+C | | |
| 990 | TAAATTTATCAGGAA | A57988Hi | +T*+A*+A*A*T*T*T*A*T*C*A*G*G*A*A*G*+ | 21330 | 19 |
| | GTGC | | T*+G*+C | | |
| 991 | TTAAATTTATCAGGA | A57989Hi | +T*+T*+A*A*A*T*T*T*A*T*C*A*G*G*A*A*+ | 21331 | 19 |
| | AGTG | | G*+T*+G | | |
| 992 | AAATAGCCATGTATT | A57990Hi | +A*+A*+A*T*A*G*C*C*A*T*G*T*A*T*T*A*+ | 21495 | 19 |
| | AATC | | A*+T*+C | | |
| 993 | GCAGTTGGTTTAGAC | A57991Hi | +G*+C*+A*G*T*T*G*G*T*T*T*A*G*A*C*+T* | 21551 | 18 |
| | TCC | | +C*+C | | |
| 994 | AGCAGTTGGTTTAGA | A57992Hi | +A*+G*+C*A*G*T*T*G*G*T*T*T*A*G*A*C*+ | 21551 | 19 |
| | CTCC | | T*+C*+C | | |
| 995 | AGAAGCAGTTGGTTT | A57993Hi | +A*+G*+A*A*G*C*A*G*T*T*G*G*T*T*T*A*+ | 21554 | 19 |
| | AGAC | | G*+A*+C | | |
| 996 | TAGAAGCAGTTGGTT | A57994Hi | +T*+A*+G*A*A*G*C*A*G*T*T*G*G*T*T*T*+ | 21555 | 19 |
| | TAGA | | A*+G*+A | | |
| 997 | TAGAAGCAGTTGGTT | A57995Hi | +T*+A*+G*A*A*G*C*A*G*T*T*G*G*T*T*+T* | 21556 | 18 |
| | TAG | | +A*+G | | |
| 998 | CTAGAAGCAGTTGGT | A57996Hi | +C*+T*+A*G*A*A*G*C*A*G*T*T*G*G*T*+T* | 21557 | 18 |
| | TTA | | +T*+A | | |
| 999 | GTCAGCCTAGAAGCA | A57997Hi | +G*+T*+C*A*G*C*C*T*A*G*A*A*G*C*A*G*+ | 21562 | 19 |
| | GTTG | | T*+T*+G | | |
| 1000 | AGTCAGCCTAGAAGC | A57998Hi | +A*+G*+T*C*A*G*C*C*T*A*G*A*A*G*C*A*+ | 21563 | 19 |
| | AGTT | | G*+T*+T | | |
| 1001 | GTCAGCCTAGAAGCA | A57999Hi | +G*+T*+C*A*G*C*C*T*A*G*A*A*G*C*A*+G* | 21563 | 18 |
| | GTT | | +T*+T | | |
| 1002 | GCAGTCAGCCTAGAA | A571000Hi | +G*+C*+A*G*T*C*A*G*C*C*T*A*G*A*A*+G* | 21566 | 18 |
| | GCA | | +C*+A | | |
| 1003 | AATTTAAGTCCTCAG | A571001Hi | +A*+A*+T*T*T*A*A*G*T*C*C*T*C*A*G*G*+ | 21640 | 19 |
| | GATG | | A*+T*+G | | |
| 1004 | TAATTTAAGTCCTCA | A571002Hi | +T*+A*+A*T*T*T*A*A*G*T*C*C*T*C*A*G*+ | 21641 | 19 |
| | GGAT | | G*+A*+T | | |
| 1005 | CTTTAATTTAAGTCCT | A571003Hi | +C*+T*+T*T*A*A*T*T*T*A*A*G*T*C*C*T*+ | 21644 | 19 |
| | CAG | | C*+A*+G | | |
| 1006 | CATCTTTAATTTAAGT | A571004Hi | +C*+A*+T*C*T*T*T*A*A*T*T*T*A*A*G*T*+ | 21647 | 19 |
| | CCT | | C*+C*+T | | |
| 1007 | AGCATCTTTAATTTAA | A571005Hi | +A*+G*+C*A*T*C*T*T*T*A*A*T*T*T*A*A*+ | 21649 | 19 |
| | GTC | | G*+T*+C | | |
| 1008 | AGCATCTTTAATTTAA | A571006Hi | +A*+G*+C*A*T*C*T*T*T*A*A*T*T*T*A*+A* | 21650 | 18 |
| | GT | | +G*+T | | |
| 1009 | CTCTGAAAGAGCATC | A571007Hi | +C*+T*+C*T*G*A*A*A*G*A*G*C*A*T*C*T*+ | 21658 | 19 |
| | TTTA | | T*+T*+A | | |
| 1010 | TCAGTCCTGTGCTCT | A571008Hi | +T*+C*+A*G*T*C*C*T*G*T*G*C*T*C*T*G*+ | 21689 | 19 |
| | GACT | | A*+C*+T | | |
| 1011 | GACTCTCTGCAAGGT | A571009Hi | +G*+A*+C*T*C*T*C*T*G*C*A*A*G*G*T*A*+ | 21763 | 19 |
| | AAGG | | A*+G*+G | | |
| 1012 | TTCAGACTCTCTGCA | A571010Hi | +T*+T*+C*A*G*A*C*T*C*T*C*T*G*C*A*A*+ | 21767 | 19 |
| | AGGT | | G*+G*+T | | |
| 1013 | AGCTGTGAGTTGAAC | A571011Hi | +A*+G*+C*T*G*T*G*A*G*T*T*G*A*A*C*+G* | 21839 | 18 |
| | GCC | | +C*+C | | |
| 1014 | GAGCTGTGAGTTGAA | A571012Hi | +G*+A*+G*C*T*G*T*G*A*G*T*T*G*A*A*+C* | 21840 | 18 |
| | CGC | | +G*+C | | |
| 1015 | AAACAAGAGCTGTGA | A571013Hi | +A*+A*+A*C*A*A*G*A*G*C*T*G*T*G*A*G*+ | 21845 | 19 |
| | GTTG | | T*+T*+G | | |
| 1016 | AACAAGAGCTGTGAG | A571014Hi | +A*+A*+C*A*A*G*A*G*C*T*G*T*G*A*G*+T* | 21845 | 18 |
| | TTG | | +T*+G | | |
| 1017 | GCATTGTTGTCCTCA | A571015Hi | +G*+C*+A*T*T*G*T*T*G*T*C*C*T*C*A*C*+ | 21924 | 19 |
| | CAAA | | A*+A*+A | | |
| 1018 | AAATATTCCTGGTTA | A571016Hi | +A*+A*+A*T*A*T*T*C*C*T*G*G*T*T*A*T*+ | 22024 | 19 |
| | TCCT | | C*+C*+T | | |
| 1019 | GCCTATTATAATAAT | A571017Hi | +G*+C*+C*T*A*T*T*A*T*A*A*T*A*A*T*C*+ | 22053 | 19 |
| | CCTA | | C*+T*+A | | |
| 1020 | GGATATAATCATGAT | A571018Hi | +G*+G*+A*T*A*T*A*A*T*C*A*T*G*A*T*A*+ | 22170 | 19 |
| | ACTA | | C*+T*+A | | |
| 1021 | GGATATAATCATGAT | A571019Hi | +G*+G*+A*T*A*T*A*A*T*C*A*T*G*A*T*+A* | 22171 | 18 |
| | ACT | | +C*+T | | |
| 1022 | CAAGTTGTAACTTGA | A571020Hi | +C*+A*+A*G*T*T*G*T*A*A*C*T*T*G*A*C*+ | 23204 | 19 |
| | CTGT | | T*+G*+T | | |
| 1023 | CAAGTTGTAACTTGA | A571021Hi | +C*+A*+A*G*T*T*G*T*A*A*C*T*T*G*A*+C* | 23205 | 18 |
| | CTG | | +T*+G | | |
| 1024 | GCAAGTTGTAACTTG | A571022Hi | +G*+C*+A*A*G*T*T*G*T*A*A*C*T*T*+G*+A | 23207 | 17 |
| | AC | | *+C | | |
| 1025 | TGCAAGTTGTAACTT | A571023Hi | +T*+G*+C*A*A*G*T*T*G*T*A*A*C*T*T*+G* | 23207 | 18 |
| | GAC | | +A*+C | | |
| 1026 | TAATGTAACATGTGC | A571024Hi | +T*+A*+A*T*G*T*A*A*C*A*T*G*T*G*C*A*+ | 23218 | 19 |
| | AAGT | | A*+G*+T | | |
| 1027 | TTGGCAGTCAAGTAG | A571025Hi | +T*+T*+G*G*C*A*G*T*C*A*A*G*T*A*G*C*+ | 23306 | 19 |
| | CATT | | A*+T*+T | | |
| 1028 | ATTTGGCAGTCAAGT | A571026Hi | +A*+T*+T*T*G*G*C*A*G*T*C*A*A*G*T*A*+ | 23308 | 19 |
| | AGCA | | G*+C*+A | | |
| 1029 | CAATTTGGCAGTCAA | A571027Hi | +C*+A*+A*T*T*T*G*G*C*A*G*T*C*A*A*G*+ | 23310 | 19 |
| | GTAG | | T*+A*+G | | |
| 1030 | ACCAGCTTGTCTAAA | A571028Hi | +A*+C*+C*A*G*C*T*T*G*T*C*T*A*A*A*C*+ | 23833 | 19 |
| | CTTG | | T*+T*+G | | |
| 1031 | ACCAGCTTGTCTAAA | A571029Hi | +A*+C*+C*A*G*C*T*T*G*T*C*T*A*A*A*+C* | 23834 | 18 |
| | CTT | | +T*+T | | |
| 1032 | TTACCAGCTTGTCTA | A571030Hi | +T*+T*+A*C*C*A*G*C*T*T*G*T*C*T*A*+A* | 23836 | 18 |
| | AAC | | +A*+C | | |
| 1033 | TTTACCAGCTTGTCT | A571031Hi | +T*+T*+T*A*C*C*A*G*C*T*T*G*T*C*T*A*+ | 23836 | 19 |
| | AAAC | | A*+A*+C | | |
| 1034 | AGCTCCTTTACCAAC | A571032Hi | +A*+G*+C*T*C*C*T*T*T*A*C*C*A*A*C*A*+ | 23951 | 19 |
| | ACCT | | C*+C*+T | | |
| 1035 | GAGCTCCTTTACCAA | A571033Hi | +G*+A*+G*C*T*C*C*T*T*T*A*C*C*A*A*C*+ | 23952 | 19 |
| | CACC | | A*+C*+C | | |
| 1036 | AGCTCCTTTACCAAC | A571034Hi | +A*+G*+C*T*C*C*T*T*T*A*C*C*A*A*C*+A* | 23952 | 18 |
| | ACC | | +C*+C | | |
| 1037 | GGAGCTCCTTTACCA | A571035Hi | +G*+G*+A*G*C*T*C*C*T*T*T*A*C*C*A*A*+ | 23953 | 19 |
| | ACAC | | C*+A*+C | | |
| 1038 | GAGCTCCTTTACCAA | A571036Hi | +G*+A*+G*C*T*C*C*T*T*T*A*C*C*A*A*+C* | 23953 | 18 |
| | CAC | | +A*+C | | |
| 1039 | TGGAGCTCCTTTACC | A571037Hi | +T*+G*+G*A*G*C*T*C*C*T*T*T*A*C*C*A*+ | 23954 | 19 |
| | AACA | | A*+C*+A | | |
| 1040 | ATGGAGCTCCTTTAC | A571038Hi | +A*+T*+G*G*A*G*C*T*C*C*T*T*T*A*C*C*+ | 23955 | 19 |
| | CAAC | | A*+A*+C | | |
| 1041 | TGGAGCTCCTTTACC | A571039Hi | +T*+G*+G*A*G*C*T*C*C*T*T*T*A*C*C*+A* | 23955 | 18 |
| | AAC | | +A*+C | | |
| 1042 | TATGGAGCTCCTTTA | A571040Hi | +T*+A*+T*G*G*A*G*C*T*C*C*T*T*T*A*C*+ | 23956 | 19 |
| | CCAA | | C*+A*+A | | |
| 1043 | ATGGAGCTCCTTTAC | A571041Hi | +A*+T*+G*G*A*G*C*T*C*C*T*T*T*A*C*+C* | 23956 | 18 |
| | CAA | | +A*+A | | |
| 1044 | AACGGCCTCACAATC | A571042Hi | +A*+A*+C*G*G*C*C*T*C*A*C*A*A*T*C*+A* | 23991 | 18 |
| | AAA | | +A*+A | | |
| 1045 | TCAACGGCCTCACAA | A571043Hi | +T*+C*+A*A*C*G*G*C*C*T*C*A*C*A*A*+T* | 23993 | 18 |
| | TCA | | +C*+A | | |
| 1046 | TCAACGGCCTCACAA | A571044Hi | +T*+C*+A*A*C*G*G*C*C*T*C*A*C*A*+A*+T | 23994 | 17 |
| | TC | | *+C | | |
| 1047 | TCATCAACGGCCTCA | A571045Hi | +T*+C*+A*T*C*A*A*C*G*G*C*C*T*C*A*+C* | 23996 | 18 |
| | CAA | | +A*+A | | |
| 1048 | TTCATCAACGGCCTC | A571046Hi | +T*+T*+C*A*T*C*A*A*C*G*G*C*C*T*C*+A* | 23997 | 18 |
| | ACA | | +C*+A | | |
| 1049 | TATTCATCAACGGCC | A571047Hi | +T*+A*+T*T*C*A*T*C*A*A*C*G*G*C*C*+T* | 23999 | 18 |
| | TCA | | +C*+A | | |
| 1050 | TATTCATCAACGGCC | A571048Hi | +T*+A*+T*T*C*A*T*C*A*A*C*G*G*C*+C*+T | 24000 | 17 |
| | TC | | *+C | | |
| 1051 | ATATTCATCAACGGC | A571049Hi | +A*+T*+A*T*T*C*A*T*C*A*A*C*G*G*+C*+C | 24001 | 17 |
| | CT | | *+T | | |
| 1052 | AATATTCATCAACGG | A571050Hi | +A*+A*+T*A*T*T*C*A*T*C*A*A*C*G*G*+C* | 24001 | 18 |
| | CCT | | +C*+T | | |
| 1053 | AAGTGATCTCTGAGA | A571051Hi | +A*+A*+G*T*G*A*T*C*T*C*T*G*A*G*A*T*+ | 24071 | 19 |
| | TACA | | A*+C*+A | | |
| 1054 | ACTAAGTGATCTCTG | A571052Hi | +A*+C*+T*A*A*G*T*G*A*T*C*T*C*T*G*A*+ | 24074 | 19 |
| | AGAT | | G*+A*+T | | |
| 1055 | AGTGTAACTAAGTGA | A571053Hi | +A*+G*+T*G*T*A*A*C*T*A*A*G*T*G*A*T*+ | 24080 | 19 |
| | TCTC | | C*+T*+C | | |
| 1056 | CTATAAGTGTAACTA | A571054Hi | +C*+T*+A*T*A*A*G*T*G*T*A*A*C*T*+A*+A | 24087 | 17 |
| | AG | | *+G | | |
| 1057 | TCTATAAGTGTAACT | A571055Hi | +T*+C*+T*A*T*A*A*G*T*G*T*A*A*C*T*+A* | 24087 | 18 |
| | AAG | | +A*+G | | |
| 1058 | GTCTAAATCCTTATG | A571056Hi | +G*+T*+C*T*A*A*A*T*C*C*T*T*A*T*G*A*+ | 24217 | 19 |
| | AGGA | | G*+G*+A | | |
| 1059 | CTTCTGAGAATTAAT | A571057Hi | +C*+T*+T*C*T*G*A*G*A*A*T*T*A*A*T*A*+ | 24245 | 19 |
| | ATGA | | T*+G*+A | | |
| 1060 | TATTTCACCACCTCC | A571058Hi | +T*+A*+T*T*T*C*A*C*C*A*C*C*T*C*C*A*+ | 24302 | 19 |
| | AACC | | A*+C*+C | | |
| 1061 | GTCTATGATGATCTC | A571059Hi | +G*+T*+C*T*A*T*G*A*T*G*A*T*C*T*C*A*+ | 24336 | 19 |
| | AGAA | | G*+A*+A | | |
| 1062 | TGTCTATGATGATCT | A571060Hi | +T*+G*+T*C*T*A*T*G*A*T*G*A*T*C*T*C*+ | 24337 | 19 |
| | CAGA | | A*+G*+A | | |
| 1063 | GATCTTGTCTATGAT | A571061Hi | +G*+A*+T*C*T*T*G*T*C*T*A*T*G*A*T*G*+ | 24342 | 19 |
| | GATC | | A*+T*+C | | |
| 1064 | TCACTTAAAGGAATA | A571062Hi | +T*+C*+A*C*T*T*A*A*A*G*G*A*A*T*A*+G* | 24395 | 18 |
| | GTG | | +T*+G | | |
| 1065 | GTTAAATTACCTTTAA | A571063Hi | +G*+T*+T*A*A*A*T*T*A*C*C*T*T*T*A*A*+ | 24768 | 19 |
| | CAT | | C*+A*+T | | |
| 1066 | CATCCATGGAAGTTA | A571064Hi | +C*+A*+T*C*C*A*T*G*G*A*A*G*T*T*A*A*+ | 24779 | 19 |
| | AATT | | A*+T*+T | | |
| 1067 | CGTTTAATACCAGGA | A571065H | +C*+G*+T*T*T*A*A*T*A*C*C*A*G*G*A*T*+ | 24841 | 19 |
| | TATT | | A*+T*+T | | |
| 1068 | CGTTTAATACCAGGA | A571066H | +C*+G*+T*T*T*A*A*T*A*C*C*A*G*G*A*+T* | 24842 | 18 |
| | TAT | | +A*+T | | |
| 1069 | TCGTTTAATACCAGG | A571067H | +T*+C*+G*T*T*T*A*A*T*A*C*C*A*G*G*A*+ | 24842 | 19 |
| | ATAT | | T*+A*+T | | |
| 1070 | CGTTTAATACCAGGA | A571068H | +C*+G*+T*T*T*A*A*T*A*C*C*A*G*G*+A*+T | 24843 | 17 |
| | TA | | *+A | | |
| 1071 | TCGTTTAATACCAGG | A571069H | +T*+C*+G*T*T*T*A*A*T*A*C*C*A*G*G*+A* | 24843 | 18 |
| | ATA | | +T*+A | | |
| 1072 | TTCGTTTAATACCAG | A571070H | +T*+T*+C*G*T*T*T*A*A*T*A*C*C*A*G*G*+ | 24843 | 19 |
| | GATA | | A*+T*+A | | |
| 1073 | CTTCGTTTAATACCA | A571071H | +C*+T*+T*C*G*T*T*T*A*A*T*A*C*C*A*G*+ | 24844 | 19 |
| | GGAT | | G*+A*+T | | |
| 1074 | GACTTCGTTTAATAC | A571072H | +G*+A*+C*T*T*C*G*T*T*T*A*A*T*A*C*C*+ | 24846 | 19 |
| | CAGG | | A*+G*+G | | |
| 1075 | GGACTTCGTTTAATA | A571073H | +G*+G*+A*C*T*T*C*G*T*T*T*A*A*T*A*+C* | 24848 | 18 |
| | CCA | | +C*+A | | |
| 1076 | TGGACTTCGTTTAAT | A571074H | +T*+G*+G*A*C*T*T*C*G*T*T*T*A*A*T*A*+ | 24848 | 19 |
| | ACCA | | C*+C*+A | | |
| 1077 | GGACTTCGTTTAATA | A571075H | +G*+G*+A*C*T*T*C*G*T*T*T*A*A*T*+A*+C | 24849 | 17 |
| | CC | | *+C | | |
| 1078 | TGGACTTCGTTTAAT | A571076H | +T*+G*+G*A*C*T*T*C*G*T*T*T*A*A*T*+A* | 24849 | 18 |
| | ACC | | +C*+C | | |
| 1079 | CATGGACTTCGTTTA | A571077H | +C*+A*+T*G*G*A*C*T*T*C*G*T*T*T*A*A*+ | 24850 | 19 |
| | ATAC | | T*+A*+C | | |
| 1080 | CATGGACTTCGTTTA | A571078H | +C*+A*+T*G*G*A*C*T*T*C*G*T*T*T*A*+A* | 24851 | 18 |
| | ATA | | +T*+A | | |
| 1081 | TCATGGACTTCGTTT | A571079H | +T*+C*+A*T*G*G*A*C*T*T*C*G*T*T*T*A*+ | 24851 | 19 |
| | AATA | | A*+T*+A | | |
| 1082 | TCATGGACTTCGTTT | A571080H | +T*+C*+A*T*G*G*A*C*T*T*C*G*T*T*T*+A* | 24852 | 18 |
| | AAT | | +A*+T | | |
| 1083 | GCACAGCTTTCACCA | A571081H | +G*+C*+A*C*A*G*C*T*T*T*C*A*C*C*A*C*+ | 24871 | 19 |
| | CAAA | | A*+A*+A | | |
| 1084 | CCAAACAGCTGAGCG | A571082H | +C*+C*+A*A*A*C*A*G*C*T*G*A*G*C*G*C*+ | 24922 | 19 |
| | CTGA | | T*+G*+A | | |
| 1085 | CCAAACAGCTGAGCG | A571083H | +C*+C*+A*A*A*C*A*G*C*T*G*A*G*C*G*+C* | 24923 | 18 |
| | CTG | | +T*+G | | |
| 1086 | GCCAAACAGCTGAGC | A571084H | +G*+C*+C*A*A*A*C*A*G*C*T*G*A*G*C*G* | 24923 | 19 |
| | GCTG | | +C*+T*+G | | |
| 1087 | GGGCCAAACAGCTGA | A571085H | +G*+G*+G*C*C*A*A*A*C*A*G*C*T*G*A*G* | 24925 | 19 |
| | GCGC | | +C*+G*+C | | |
| 1088 | GGGCCAAACAGCTGA | A571086H | +G*+G*+G*C*C*A*A*A*C*A*G*C*T*G*A*+G | 24926 | 18 |
| | GCG | | *+C*+G | | |
| 1089 | AGGGCCAAACAGCTG | A571087H | +A*+G*+G*G*C*C*A*A*A*C*A*G*C*T*G*A* | 24926 | 19 |
| | AGCG | | +G*+C*+G | | |
| 1090 | TGATTCTCTTGTTACA | A571088Hi | +T*+G*+A*T*T*C*T*C*T*T*G*T*T*A*C*A*+ | 25007 | 19 |
| | ATG | | A*+T*+G | | |
| 1091 | ATGATTCTCTTGTTAC | A571089Hi | +A*+T*+G*A*T*T*C*T*C*T*T*G*T*T*A*C*+ | 25008 | 19 |
| | AAT | | A*+A*+T | | |
| 1092 | ATGATTCTCTTGTTAC | A571090Hi | +A*+T*+G*A*T*T*C*T*C*T*T*G*T*T*A*+C* | 25009 | 18 |
| | AA | | +A*+A | | |
| 1093 | TATGATTCTCTTGTTA | A571091Hi | +T*+A*+T*G*A*T*T*C*T*C*T*T*G*T*T*A*+ | 25009 | 19 |
| | CAA | | C*+A*+A | | |
| 1094 | ATATGATTCTCTTGTT | A571092Hi | +A*+T*+A*T*G*A*T*T*C*T*C*T*T*G*T*T*+ | 25010 | 19 |
| | ACA | | A*+C*+A | | |
| 1095 | ACATATGATTCTCTT | A571093Hi | +A*+C*+A*T*A*T*G*A*T*T*C*T*C*T*T*G*+ | 25012 | 19 |
| | GTTA | | T*+T*+A | | |
| 1096 | GTACATATGATTCTC | A571094Hi | +G*+T*+A*C*A*T*A*T*G*A*T*T*C*T*C*T*+ | 25014 | 19 |
| | TTGT | | T*+G*+T | | |
| 1097 | CCATCTGTACATATG | A571095Hi | +C*+C*+A*T*C*T*G*T*A*C*A*T*A*T*G*A*+ | 25020 | 19 |
| | ATTC | | T*+T*+C | | |
| 1098 | CAAGAATGATGTAAA | A571096Hi | +C*+A*+A*G*A*A*T*G*A*T*G*T*A*A*A*G*+ | 25143 | 19 |
| | GCAA | | C*+A*+A | | |
| 1099 | GACAAAGCTAAATCA | A571097Hi | +G*+A*+C*A*A*A*G*C*T*A*A*A*T*C*A*A*+ | 25156 | 19 |
| | AGAA | | G*+A*+A | | |
| 1100 | GCTGCTTAATGACAA | A571098Hi | +G*+C*+T*G*C*T*T*A*A*T*G*A*C*A*A*A*+ | 25166 | 19 |
| | AGCT | | G*+C*+T | | |
| 1101 | CCTTGGTCAATTATA | A571099Hi | +C*+C*+T*T*G*G*T*C*A*A*T*T*A*T*A*A*+ | 26097 | 19 |
| | AAGA | | A*+G*+A | | |
| 1102 | AGAATGAGGTGTGCT | A571100Hi | +A*+G*+A*A*T*G*A*G*G*T*G*T*G*C*T*T*+ | 26122 | 19 |
| | TTAG | | T*+A*+G | | |
| 1103 | GAATGAGGTGTGCTT | A571101Hi | +G*+A*+A*T*G*A*G*G*T*G*T*G*C*T*T*+T* | 26122 | 18 |
| | TAG | | +A*+G | | |
| 1104 | AACAGAATGAGGTGT | A571102Hi | +A*+A*+C*A*G*A*A*T*G*A*G*G*T*G*T*G*+ | 26125 | 19 |
| | GCTT | | C*+T*+T | | |
| 1105 | CAACAGAATGAGGTG | A571103Hi | +C*+A*+A*C*A*G*A*A*T*G*A*G*G*T*G*T*+ | 26126 | 19 |
| | TGCT | | G*+C*+T | | |
| 1106 | TACCAACAGAATGAG | A571104Hi | +T*+A*+C*C*A*A*C*A*G*A*A*T*G*A*G*G*+ | 26129 | 19 |
| | GTGT | | T*+G*+T | | |
| 1107 | ACAGTTTGACCAACT | A571105Hi | +A*+C*+A*G*T*T*T*G*A*C*C*A*A*C*T*C*+ | 26166 | 19 |
| | CTAA | | T*+A*+A | | |
| 1108 | CTTAACAGTTTGACC | A571106Hi | +C*+T*+T*A*A*C*A*G*T*T*T*G*A*C*C*A*+ | 26170 | 19 |
| | AACT | | A*+C*+T | | |
| 1109 | CTTAACAGTTTGACC | A571107Hi | +C*+T*+T*A*A*C*A*G*T*T*T*G*A*C*C*+A* | 26171 | 18 |
| | AAC | | +A*+C | | |
| 1110 | CCTCTAAATCTTGATT | A571108H | +C*+C*+T*C*T*A*A*A*T*C*T*T*G*A*T*T*+ | 26250 | 19 |
| | TAA | | T*+A*+A | | |
| 1111 | CATCATCATTCTCTTG | A571109H | +C*+A*+T*C*A*T*C*A*T*T*C*T*C*T*T*G*+ | 26280 | 19 |
| | ATT | | A*+T*+T | | |
| 1112 | CTCATCATCATTCTCT | A571110H | +C*+T*+C*A*T*C*A*T*C*A*T*T*C*T*C*T*+ | 26282 | 19 |
| | TGA | | T*+G*+A | | |
| 1113 | ACAGGCTTCCAGCCA | A571111H | +A*+C*+A*G*G*C*T*T*C*C*A*G*C*C*A*A*+ | 26318 | 19 |
| | AAAC | | A*+A*+C | | |
| 1114 | TAACAGGCTTCCAGC | A571112H | +T*+A*+A*C*A*G*G*C*T*T*C*C*A*G*C*C*+ | 26320 | 19 |
| | CAAA | | A*+A*+A | | |
| 1115 | TTGTAACAGGCTTCC | A571113H | +T*+T*+G*T*A*A*C*A*G*G*C*T*T*C*C*A*+ | 26323 | 19 |
| | AGCC | | G*+C*+C | | |
| 1116 | CTTTGTAACAGGCTT | A571114H | +C*+T*+T*T*G*T*A*A*C*A*G*G*C*T*T*C*+ | 26325 | 19 |
| | CCAG | | C*+A*+G | | |
| 1117 | GCTTTGTAACAGGCT | A571115H | +G*+C*+T*T*T*G*T*A*A*C*A*G*G*C*T*T*+ | 26326 | 19 |
| | TCCA | | C*+C*+A | | |
| 1118 | TCTTTCTATGCCACG | A571116H | +T*+C*+T*T*T*C*T*A*T*G*C*C*A*C*G*T*+ | 26346 | 19 |
| | TTAA | | T*+A*+A | | |
| 1119 | TCTTTCTATGCCACG | A571117H | +T*+C*+T*T*T*C*T*A*T*G*C*C*A*C*G*+T* | 26347 | 18 |
| | TTA | | +T*+A | | |
| 1120 | TTCTTTCTATGCCAC | A571118H | +T*+T*+C*T*T*T*C*T*A*T*G*C*C*A*C*G*+ | 26347 | 19 |
| | GTTA | | T*+T*+A | | |
| 1121 | TTCTTTCTATGCCAC | A571119H | +T*+T*+C*T*T*T*C*T*A*T*G*C*C*A*C*+G* | 26348 | 18 |
| | GTT | | +T*+T | | |
| 1122 | GTTCTTTCTATGCCA | A571120H | +G*+T*+T*C*T*T*T*C*T*A*T*G*C*C*A*C*+ | 26348 | 19 |
| | CGTT | | G*+T*+T | | |
| 1123 | TGTTCTTTCTATGCCA | A571121H | +T*+G*+T*T*C*T*T*T*C*T*A*T*G*C*C*A*+ | 26349 | 19 |
| | CGT | | C*+G*+T | | |
| 1124 | TGTTCTTTCTATGCCA | A571122H | +T*+G*+T*T*C*T*T*T*C*T*A*T*G*C*C*+A* | 26350 | 18 |
| | CG | | +C*+G | | |
| 1125 | TTGTTCTTTCTATGCC | A571123H | +T*+T*+G*T*T*C*T*T*T*C*T*A*T*G*C*C*+ | 26350 | 19 |
| | ACG | | A*+C*+G | | |
| 1126 | CGTGCTTGTTCTTTCT | A571124H | +C*+G*+T*G*C*T*T*G*T*T*C*T*T*T*C*T*+ | 26355 | 19 |
| | ATG | | A*+T*+G | | |
| 1127 | CACGTGCTTGTTCTT | A571125H | +C*+A*+C*G*T*G*C*T*T*G*T*T*C*T*T*T*+ | 26357 | 19 |
| | TCTA | | C*+T*+A | | |
| 1128 | CACGTGCTTGTTCTT | A571126H | +C*+A*+C*G*T*G*C*T*T*G*T*T*C*T*T*+T* | 26358 | 18 |
| | TCT | | +C*+T | | |
| 1129 | GCACGTGCTTGTTCT | A571127H | +G*+C*+A*C*G*T*G*C*T*T*G*T*T*C*T*T*+ | 26358 | 19 |
| | TTCT | | T*+C*+T | | |
| 1130 | CACGTGCTTGTTCTT | A571128H | +C*+A*+C*G*T*G*C*T*T*G*T*T*C*T*+T*+T | 26359 | 17 |
| | TC | | *+C | | |
| 1131 | GCACGTGCTTGTTCT | A571129H | +G*+C*+A*C*G*T*G*C*T*T*G*T*T*C*T*+T* | 26359 | 18 |
| | TTC | | +T*+C | | |
| 1132 | TGCACGTGCTTGTTC | A571130H | +T*+G*+C*A*C*G*T*G*C*T*T*G*T*T*C*T*+ | 26359 | 19 |
| | TTTC | | T*+T*+C | | |
| 1133 | AACTCTAATATTTATG | A571131Hi | +A*+A*+C*T*C*T*A*A*T*A*T*T*T*A*T*G*+ | 26386 | 19 |
| | AGA | | A*+G*+A | | |
| 1134 | ATTATTTAGTGCCCA | A571132H | +A*+T*+T*A*T*T*T*A*G*T*G*C*C*C*A*G*+ | 26477 | 19 |
| | GCAT | | C*+A*+T | | |
| 1135 | GATTATTTAGTGCCC | A571133H | +G*+A*+T*T*A*T*T*T*A*G*T*G*C*C*C*A*+ | 26478 | 19 |
| | AGCA | | G*+C*+A | | |
| 1136 | GATTATTTAGTGCCC | A571134H | +G*+A*+T*T*A*T*T*T*A*G*T*G*C*C*C*+A* | 26479 | 18 |
| | AGC | | +G*+C | | |
| 1137 | AGATTATTTAGTGCC | A571135H | +A*+G*+A*T*T*A*T*T*T*A*G*T*G*C*C*C*+ | 26479 | 19 |
| | CAGC | | A*+G*+C | | |
| 1138 | AGGAGATTATTTAGT | A571136H | +A*+G*+G*A*G*A*T*T*A*T*T*T*A*G*T*G*+ | 26482 | 19 |
| | GCCC | | C*+C*+C | | |
| 1139 | AAGGAGATTATTTAG | A571137H | +A*+A*+G*G*A*G*A*T*T*A*T*T*T*A*G*T*+ | 26483 | 19 |
| | TGCC | | G*+C*+C | | |
| 1140 | ACATAAGGAGATTAT | A571138H | +A*+C*+A*T*A*A*G*G*A*G*A*T*T*A*T*T*+ | 26487 | 19 |
| | TTAG | | T*+A*+G | | |
| 1141 | GGTACATAAGGAGAT | A571139H | +G*+G*+T*A*C*A*T*A*A*G*G*A*G*A*T*T*+ | 26490 | 19 |
| | TATT | | A*+T*+T | | |
| 1142 | TTTGGTACATAAGGA | A571140H | +T*+T*+T*G*G*T*A*C*A*T*A*A*G*G*A*G*+ | 26493 | 19 |
| | GATT | | A*+T*+T | | |
| 1143 | TTTTGGTACATAAGG | A571141H | +T*+T*+T*T*G*G*T*A*C*A*T*A*A*G*G*A*+ | 26494 | 19 |
| | AGAT | | G*+A*+T | | |
| 1144 | GTTTTGGTACATAAG | A571142H | +G*+T*+T*T*T*G*G*T*A*C*A*T*A*A*G*G*+ | 26495 | 19 |
| | GAGA | | A*+G*+A | | |
| 1145 | TGTTTTGGTACATAA | A571143H | +T*+G*+T*T*T*T*G*G*T*A*C*A*T*A*A*G*+ | 26496 | 19 |
| | GGAG | | G*+A*+G | | |
| 1146 | CTGTTTTGGTACATA | A571144H | +C*+T*+G*T*T*T*T*G*G*T*A*C*A*T*A*A*+ | 26497 | 19 |
| | AGGA | | G*+G*+A | | |
| 1147 | AACTGTTTTGGTACA | A571145H | +A*+A*+C*T*G*T*T*T*T*G*G*T*A*C*A*T*+ | 26499 | 19 |
| | TAAG | | A*+A*+G | | |
| 1148 | TGTAAACTGTTTTGG | A571146H | +T*+G*+T*A*A*A*C*T*G*T*T*T*T*G*G*T*+ | 26503 | 19 |
| | TACA | | A*+C*+A | | |
| 1149 | CATGTAAACTGTTTT | A571147H | +C*+A*+T*G*T*A*A*A*C*T*G*T*T*T*T*G*+ | 26505 | 19 |
| | GGTA | | G*+T*+A | | |
| 1150 | CAATCTTCTCATGTA | A571148H | +C*+A*+A*T*C*T*T*C*T*C*A*T*G*T*A*A*+ | 26514 | 19 |
| | AACT | | A*+C*+T | | |
| 1151 | CCAATCTTCTCATGT | A571149H | +C*+C*+A*A*T*C*T*T*C*T*C*A*T*G*T*A*+ | 26515 | 19 |
| | AAAC | | A*+A*+C | | |
| 1152 | TCCAATCTTCTCATGT | A571150H | +T*+C*+C*A*A*T*C*T*T*C*T*C*A*T*G*T*+ | 26516 | 19 |
| | AAA | | A*+A*+A | | |
| 1153 | CTCCAATCTTCTCAT | A571151H | +C*+T*+C*C*A*A*T*C*T*T*C*T*C*A*T*G*+ | 26517 | 19 |
| | GTAA | | T*+A*+A | | |
| 1154 | CATCTCCAATCTTCTC | A571152H | +C*+A*+T*C*T*C*C*A*A*T*C*T*T*C*T*C*+ | 26520 | 19 |
| | ATG | | A*+T*+G | | |
| 1155 | TCATCTCCAATCTTCT | A571153H | +T*+C*+A*T*C*T*C*C*A*A*T*C*T*T*C*T*+ | 26521 | 19 |
| | CAT | | C*+A*+T | | |
| 1156 | TGGCCATCTTCATCT | A571154H | +T*+G*+G*C*C*A*T*C*T*T*C*A*T*C*T*C*+ | 26530 | 19 |
| | CCAA | | C*+A*+A | | |
| 1157 | ATCTCTCAATAATAA | A571155Hi | +A*+T*+C*T*C*T*C*A*A*T*A*A*T*A*A*+C* | 26746 | 18 |
| | CTC | | +T*+C | | |
| 1158 | CCAAATCTCTCAATA | A571156Hi | +C*+C*+A*A*A*T*C*T*C*T*C*A*A*T*A*A*+ | 26749 | 19 |
| | ATAA | | T*+A*+A | | |
| 1159 | CTCTGCAATTTCTATC | A571157Hi | +C*+T*+C*T*G*C*A*A*T*T*T*C*T*A*T*C*+ | 26861 | 19 |
| | ACC | | A*+C*+C | | |
| 1160 | ATGGCTCTGCAATTT | A571158Hi | +A*+T*+G*G*C*T*C*T*G*C*A*A*T*T*T*C*+ | 26865 | 19 |
| | CTAT | | T*+A*+T | | |
| 1161 | ACCTGCTGGCTAGTC | A571159Hi | +A*+C*+C*T*G*C*T*G*G*C*T*A*G*T*C*A*+ | 26902 | 19 |
| | ACCA | | C*+C*+A | | |
| 1162 | ACCTGCTGGCTAGTC | A571160Hi | +A*+C*+C*T*G*C*T*G*G*C*T*A*G*T*C*+A* | 26903 | 18 |
| | ACC | | +C*+C | | |
| 1163 | TTGACAACCTGCTGG | A571161Hi | +T*+T*+G*A*C*A*A*C*C*T*G*C*T*G*G*C*+ | 26908 | 19 |
| | CTAG | | T*+A*+G | | |
| 1164 | TTGACAACCTGCTGG | A571162Hi | +T*+T*+G*A*C*A*A*C*C*T*G*C*T*G*G*+C* | 26909 | 18 |
| | CTA | | +T*+A | | |
| 1165 | TATTGACAACCTGCT | A571163Hi | +T*+A*+T*T*G*A*C*A*A*C*C*T*G*C*T*G*+ | 26910 | 19 |
| | GGCT | | G*+C*+T | | |
| 1166 | TATTGACAACCTGCT | A571164Hi | +T*+A*+T*T*G*A*C*A*A*C*C*T*G*C*T*+G* | 26911 | 18 |
| | GGC | | +G*+C | | |
| 1167 | ATATTGACAACCTGC | A571165Hi | +A*+T*+A*T*T*G*A*C*A*A*C*C*T*G*C*+T* | 26912 | 18 |
| | TGG | | +G*+G | | |
| 1168 | GATATTGACAACCTG | A571166Hi | +G*+A*+T*A*T*T*G*A*C*A*A*C*C*T*G*C*+ | 26912 | 19 |
| | CTGG | | T*+G*+G | | |
| 1169 | CTGATATTGACAACC | A571167Hi | +C*+T*+G*A*T*A*T*T*G*A*C*A*A*C*C*T*+ | 26914 | 19 |
| | TGCT | | G*+C*+T | | |
| 1170 | GTCAACTCTGTCACA | A571168Hi | +G*+T*+C*A*A*C*T*C*T*G*T*C*A*C*A*T*+ | 26975 | 19 |
| | TCAG | | C*+A*+G | | |
| 1171 | GGTCAACTCTGTCAC | A571169Hi | +G*+G*+T*C*A*A*C*T*C*T*G*T*C*A*C*A*+ | 26976 | 19 |
| | ATCA | | T*+C*+A | | |
| 1172 | CAGGTCAACTCTGTC | A571170Hi | +C*+A*+G*G*T*C*A*A*C*T*C*T*G*T*C*A*+ | 26978 | 19 |
| | ACAT | | C*+A*+T | | |
| 1173 | ACAGGTCAACTCTGT | A571171Hi | +A*+C*+A*G*G*T*C*A*A*C*T*C*T*G*T*C*+ | 26979 | 19 |
| | CACA | | A*+C*+A | | |
| 1174 | ACAGGTCAACTCTGT | A571172Hi | +A*+C*+A*G*G*T*C*A*A*C*T*C*T*G*T*+C* | 26980 | 18 |
| | CAC | | +A*+C | | |
| 1175 | GAAGACAGGTCAACT | A571173Hi | +G*+A*+A*G*A*C*A*G*G*T*C*A*A*C*T*+C* | 26984 | 18 |
| | CTG | | +T*+G | | |
| 1176 | AGAAGACAGGTCAAC | A571174Hi | +A*+G*+A*A*G*A*C*A*G*G*T*C*A*A*C*T*+ | 26984 | 19 |
| | TCTG | | C*+T*+G | | |
| 1177 | AAAGAAGACAGGTCA | A571175Hi | +A*+A*+A*G*A*A*G*A*C*A*G*G*T*C*A*A*+ | 26986 | 19 |
| | ACTC | | C*+T*+C | | |
| 1178 | GATGACAGCTTCAAA | A571176Hi | +G*+A*+T*G*A*C*A*G*C*T*T*C*A*A*A*T*+ | 27012 | 19 |
| | TAAC | | A*+A*+C | | |
| 1179 | ATGATGTTTGAATGG | A571177Hi | +A*+T*+G*A*T*G*T*T*T*G*A*A*T*G*G*C*+ | 27053 | 19 |
| | CACT | | A*+C*+T | | |
| 1180 | TTATGATGTTTGAAT | A571178Hi | +T*+T*+A*T*G*A*T*G*T*T*T*G*A*A*T*G*+ | 27055 | 19 |
| | GGCA | | G*+C*+A | | |
| 1181 | CTTATGATGTTTGAA | A571179Hi | +C*+T*+T*A*T*G*A*T*G*T*T*T*G*A*A*+T* | 27057 | 18 |
| | TGG | | +G*+G | | |
| 1182 | GTCTTATGATGTTTG | A571180Hi | +G*+T*+C*T*T*A*T*G*A*T*G*T*T*T*G*A*+ | 27058 | 19 |
| | AATG | | A*+T*+G | | |
| 1183 | TGAAACAAACCTGAT | A571181Hi | +T*+G*+A*A*A*C*A*A*A*C*C*T*G*A*T*A*+ | 27121 | 19 |
| | ATGC | | T*+G*+C | | |
| 1184 | ATGAAACAAACCTGA | A571182Hi | +A*+T*+G*A*A*A*C*A*A*A*C*C*T*G*A*T*+ | 27122 | 19 |
| | TATG | | A*+T*+G | | |
| 1185 | GAATACTACTATTTG | A571183Hi | +G*+A*+A*T*A*C*T*A*C*T*A*T*T*T*G*T*+ | 27239 | 19 |
| | TTTC | | T*+T*+C | | |
| 1186 | GATGCCAATTTAGCA | A571184Hi | +G*+A*+T*G*C*C*A*A*T*T*T*A*G*C*A*+T* | 27383 | 18 |
| | TAG | | +A*+G | | |
| 1187 | AGATGCCAATTTAGC | A571185Hi | +A*+G*+A*T*G*C*C*A*A*T*T*T*A*G*C*A*+ | 27383 | 19 |
| | ATAG | | T*+A*+G | | |
| 1188 | TAAGATGCCAATTTA | A571186Hi | +T*+A*+A*G*A*T*G*C*C*A*A*T*T*T*A*G*+ | 27385 | 19 |
| | GCAT | | C*+A*+T | | |
| 1189 | TTAAGATGCCAATTT | A571187Hi | +T*+T*+A*A*G*A*T*G*C*C*A*A*T*T*T*A*+ | 27386 | 19 |
| | AGCA | | G*+C*+A | | |
| 1190 | TTAAGATGCCAATTT | A571188Hi | +T*+T*+A*A*G*A*T*G*C*C*A*A*T*T*T*+A* | 27387 | 18 |
| | AGC | | +G*+C | | |
| 1191 | ATTAAGATGCCAATT | A571189Hi | +A*+T*+T*A*A*G*A*T*G*C*C*A*A*T*T*T*+ | 27387 | 19 |
| | TAGC | | A*+G*+C | | |
| 1192 | TATTAAGATGCCAAT | A571190Hi | +T*+A*+T*T*A*A*G*A*T*G*C*C*A*A*T*T*+ | 27388 | 19 |
| | TTAG | | T*+A*+G | | |
| 1193 | ATTAAGATGCCAATT | A571191Hi | +A*+T*+T*A*A*G*A*T*G*C*C*A*A*T*T*+T* | 27388 | 18 |
| | TAG | | +A*+G | | |
| 1194 | ATGAGATTATTAAGA | A571192Hi | +A*+T*+G*A*G*A*T*T*A*T*T*A*A*G*A*T*+ | 27395 | 19 |
| | TGCC | | G*+C*+C | | |
| 1195 | TTGCTCAACATGAGA | A571193Hi | +T*+T*+G*C*T*C*A*A*C*A*T*G*A*G*A*+T* | 27405 | 18 |
| | TTA | | +T*+A | | |
| 1196 | ATGTAAAGAGATGCT | A571194Hi | +A*+T*+G*T*A*A*A*G*A*G*A*T*G*C*T*G*+ | 27646 | 19 |
| | GAGG | | A*+G*+G | | |
| 1197 | AGATATGTAAAGAGA | A571195Hi | +A*+G*+A*T*A*T*G*T*A*A*A*G*A*G*A*T*+ | 27650 | 19 |
| | TGCT | | G*+C*+T | | |
| 1198 | CAGATATGTAAAGAG | A571196Hi | +C*+A*+G*A*T*A*T*G*T*A*A*A*G*A*G*A*+ | 27651 | 19 |
| | ATGC | | T*+G*+C | | |
| 1199 | TATCTCTAACAACAA | A571197Hi | +T*+A*+T*C*T*C*T*A*A*C*A*A*C*A*A*T*+ | 27819 | 19 |
| | TTAA | | T*+A*+A | | |
| 1200 | CACTTCCCTATGAGC | A571198H | +C*+A*+C*T*T*C*C*C*T*A*T*G*A*G*C*T*+ | 27917 | 19 |
| | TGGG | | G*+G*+G | | |
| 1201 | TCACTTCCCTATGAG | A571199H | +T*+C*+A*C*T*T*C*C*C*T*A*T*G*A*G*C*+ | 27918 | 19 |
| | CTGG | | T*+G*+G | | |
| 1202 | TCACTTCCCTATGAG | A571200H | +T*+C*+A*C*T*T*C*C*C*T*A*T*G*A*G*+C* | 27919 | 18 |
| | CTG | | +T*+G | | |
| 1203 | ATCACTTCCCTATGA | A571201H | +A*+T*+C*A*C*T*T*C*C*C*T*A*T*G*A*G*+ | 27919 | 19 |
| | GCTG | | C*+T*+G | | |
| 1204 | ATCACTTCCCTATGA | A571202H | +A*+T*+C*A*C*T*T*C*C*C*T*A*T*G*A*+G* | 27920 | 18 |
| | GCT | | +C*+T | | |
| 1205 | CATCACTTCCCTATG | A571203H | +C*+A*+T*C*A*C*T*T*C*C*C*T*A*T*G*A*+ | 27920 | 19 |
| | AGCT | | G*+C*+T | | |
| 1206 | GCATCACTTCCCTAT | A571204H | +G*+C*+A*T*C*A*C*T*T*C*C*C*T*A*T*G*+ | 27921 | 19 |
| | GAGC | | A*+G*+C | | |
| 1207 | GCATCACTTCCCTAT | A571205H | +G*+C*+A*T*C*A*C*T*T*C*C*C*T*A*T*+G* | 27922 | 18 |
| | GAG | | +A*+G | | |
| 1208 | AGCATCACTTCCCTA | A571206H | +A*+G*+C*A*T*C*A*C*T*T*C*C*C*T*A*T*+ | 27922 | 19 |
| | TGAG | | G*+A*+G | | |
| 1209 | GGAAAACTTCCTTTG | A571207H | +G*+G*+A*A*A*A*C*T*T*C*C*T*T*T*G*A*+ | 27960 | 19 |
| | ATGA | | T*+G*+A | | |
| 1210 | GGAAAACTTCCTTTG | A571208H | +G*+G*+A*A*A*A*C*T*T*C*C*T*T*T*G*+A* | 27961 | 18 |
| | ATG | | +T*+G | | |
| 1211 | TGGAAAACTTCCTTT | A571209H | +T*+G*+G*A*A*A*A*C*T*T*C*C*T*T*T*G*+ | 27961 | 19 |
| | GATG | | A*+T*+G | | |
| 1212 | TGTGAGTTTACACAT | A571210Hi | +T*+G*+T*G*A*G*T*T*T*A*C*A*C*A*T*A*+ | 28236 | 19 |
| | AGAA | | G*+A*+A | | |
| 1213 | GTGTGAGTTTACACA | A571211Hi | +G*+T*+G*T*G*A*G*T*T*T*A*C*A*C*A*T*+ | 28237 | 19 |
| | TAGA | | A*+G*+A | | |
| 1214 | ATTTGAGCAGCCTGA | A571212Hi | +A*+T*+T*T*G*A*G*C*A*G*C*C*T*G*A*+A* | 29068 | 18 |
| | ACC | | +C*+C | | |
| 1215 | AAATTTGAGCAGCCT | A571213Hi | +A*+A*+A*T*T*T*G*A*G*C*A*G*C*C*T*G*+ | 29069 | 19 |
| | GAAC | | A*+A*+C | | |
| 1216 | AATTTGAGCAGCCTG | A571214Hi | +A*+A*+T*T*T*G*A*G*C*A*G*C*C*T*G*+A* | 29069 | 18 |
| | AAC | | +A*+C | | |
| 1217 | GAAGAAATTTGAGCA | A571215Hi | +G*+A*+A*G*A*A*A*T*T*T*G*A*G*C*A*G*+ | 29073 | 19 |
| | GCCT | | C*+C*+T | | |
| 1218 | TTTCACCTAAGGATT | A571216Hi | +T*+T*+T*C*A*C*C*T*A*A*G*G*A*T*T*T*+ | 29113 | 19 |
| | TCTG | | C*+T*+G | | |
| 1219 | CCAATGCCTTTATGA | A571217Hi | +C*+C*+A*A*T*G*C*C*T*T*T*A*T*G*A*+T* | 29134 | 18 |
| | TGA | | +G*+A | | |
| 1220 | TACACTGGTCTCTGT | A571218Hi | +T*+A*+C*A*C*T*G*G*T*C*T*C*T*G*T*C*+ | 29214 | 19 |
| | CTCT | | T*+C*+T | | |
| 1221 | AGAAACATGCATGAG | A571219Hi | +A*+G*+A*A*A*C*A*T*G*C*A*T*G*A*G*G*+ | 29285 | 19 |
| | GTGG | | T*+G*+G | | |
| 1222 | AAGAAACATGCATGA | A571220Hi | +A*+A*+G*A*A*A*C*A*T*G*C*A*T*G*A*G*+ | 29286 | 19 |
| | GGTG | | G*+T*+G | | |
| 1223 | ATTATCCTTGAGAGC | A571221Hi | +A*+T*+T*A*T*C*C*T*T*G*A*G*A*G*C*A*+ | 29434 | 19 |
| | AGCC | | G*+C*+C | | |
| 1224 | TTATCCTTGAGAGCA | A571222Hi | +T*+T*+A*T*C*C*T*T*G*A*G*A*G*C*A*+G* | 29434 | 18 |
| | GCC | | +C*+C | | |
| 1225 | CATTATCCTTGAGAG | A571223Hi | +C*+A*+T*T*A*T*C*C*T*T*G*A*G*A*G*C*+ | 29435 | 19 |
| | CAGC | | A*+G*+C | | |
| 1226 | ATTATCCTTGAGAGC | A571224Hi | +A*+T*+T*A*T*C*C*T*T*G*A*G*A*G*C*+A* | 29435 | 18 |
| | AGC | | +G*+C | | |
| 1227 | CTCTTTAACTGCACA | A571225Hi | +C*+T*+C*T*T*T*A*A*C*T*G*C*A*C*A*G*+ | 29502 | 19 |
| | GTAG | | T*+A*+G | | |
| 1228 | TGTTGTCTGTACATA | A571226Hi | +T*+G*+T*T*G*T*C*T*G*T*A*C*A*T*A*G*+ | 29604 | 19 |
| | GTAA | | T*+A*+A | | |
| 1229 | CAATGTTGTCTGTAC | A571227Hi | +C*+A*+A*T*G*T*T*G*T*C*T*G*T*A*C*A*+ | 29607 | 19 |
| | ATAG | | T*+A*+G | | |
| 1230 | ATTTCCAGGTGGTTA | A571228Hi | +A*+T*+T*T*C*C*A*G*G*T*G*G*T*T*A*T*+ | 29640 | 19 |
| | TGAA | | G*+A*+A | | |
| 1231 | GATTTCCAGGTGGTT | A571229Hi | +G*+A*+T*T*T*C*C*A*G*G*T*G*G*T*T*A*+ | 29641 | 19 |
| | ATGA | | T*+G*+A | | |
| 1232 | AGGAGATTTCCAGGT | A571230Hi | +A*+G*+G*A*G*A*T*T*T*C*C*A*G*G*T*G*+ | 29645 | 19 |
| | GGTT | | G*+T*+T | | |
| 1233 | TCAAAGGAGATTTCC | A571231Hi | +T*+C*+A*A*A*G*G*A*G*A*T*T*T*C*C*A*+ | 29649 | 19 |
| | AGGT | | G*+G*+T | | |
| 1234 | GCAGTCGAAATTTCA | A571232Hi | +G*+C*+A*G*T*C*G*A*A*A*T*T*T*C*A*C*+ | 29691 | 19 |
| | CAAA | | A*+A*+A | | |
| 1235 | CAGTCGAAATTTCAC | A571233Hi | +C*+A*+G*T*C*G*A*A*A*T*T*T*C*A*C*+A* | 29691 | 18 |
| | AAA | | +A*+A | | |
| 1236 | GAAGCAGTCGAAATT | A571234Hi | +G*+A*+A*G*C*A*G*T*C*G*A*A*A*T*T*+T* | 29695 | 18 |
| | TCA | | +C*+A | | |
| 1237 | GGAAGCAGTCGAAAT | A571235Hi | +G*+G*+A*A*G*C*A*G*T*C*G*A*A*A*T*+T* | 29696 | 18 |
| | TTC | | +T*+C | | |
| 1238 | AGGAAGCAGTCGAAA | A571236Hi | +A*+G*+G*A*A*G*C*A*G*T*C*G*A*A*A*+T* | 29697 | 18 |
| | TTT | | +T*+T | | |
| 1239 | TGTAACCAGTCCTCA | A571237Hi | +T*+G*+T*A*A*C*C*A*G*T*C*C*T*C*A*G*+ | 29722 | 19 |
| | GACA | | A*+C*+A | | |
| 1240 | TCTGAATGTAACCAG | A571238Hi | +T*+C*+T*G*A*A*T*G*T*A*A*C*C*A*G*T*+ | 29728 | 19 |
| | TCCT | | C*+C*+T | | |
| 1241 | CTCTGAATGTAACCA | A571239Hi | +C*+T*+C*T*G*A*A*T*G*T*A*A*C*C*A*G*+ | 29729 | 19 |
| | GTCC | | T*+C*+C | | |
| 1242 | AGGCAGAACTCCAAA | A571240Hi | +A*+G*+G*C*A*G*A*A*C*T*C*C*A*A*A*G*+ | 29848 | 19 |
| | GACA | | A*+C*+A | | |
| 1243 | AAGGCAGAACTCCAA | A571241Hi | +A*+A*+G*G*C*A*G*A*A*C*T*C*C*A*A*A*+ | 29849 | 19 |
| | AGAC | | G*+A*+C | | |
| 1244 | CTTTATTAAGGCAGA | A571242Hi | +C*+T*+T*T*A*T*T*A*A*G*G*C*A*G*A*A*+ | 29856 | 19 |
| | ACTC | | C*+T*+C | | |
| 1245 | TTCATGCTTTATTAAG | A571243Hi | +T*+T*+C*A*T*G*C*T*T*T*A*T*T*A*A*G*+ | 29862 | 19 |
| | GCA | | G*+C*+A | | |
| 1246 | CTACTCAAGTTCTTC | A571244Hi | +C*+T*+A*C*T*C*A*A*G*T*T*C*T*T*C*A*+ | 29874 | 19 |
| | ATGC | | T*+G*+C | | |
| 1247 | GCTACTCAAGTTCTT | A571245Hi | +G*+C*+T*A*C*T*C*A*A*G*T*T*C*T*T*C*+ | 29875 | 19 |
| | CATG | | A*+T*+G | | |
| 1248 | AGCTACTCAAGTTCT | A571246Hi | +A*+G*+C*T*A*C*T*C*A*A*G*T*T*C*T*T*+ | 29876 | 19 |
| | TCAT | | C*+A*+T | | |
| 1249 | GAGCTACTCAAGTTC | A571247Hi | +G*+A*+G*C*T*A*C*T*C*A*A*G*T*T*C*T*+ | 29877 | 19 |
| | TTCA | | T*+C*+A | | |
| 1250 | GAGCTACTCAAGTTC | A571248Hi | +G*+A*+G*C*T*A*C*T*C*A*A*G*T*T*C*+T* | 29878 | 18 |
| | TTC | | +T*+C | | |
| 1251 | CAAGTGTCTTTCAAT | A571249Hi | +C*+A*+A*G*T*G*T*C*T*T*T*C*A*A*T*G*+ | 29920 | 19 |
| | GAGA | | A*+G*+A | | |
| 1252 | CTGCCTATTCACAAC | A571250Hi | +C*+T*+G*C*C*T*A*T*T*C*A*C*A*A*C*T*+ | 30288 | 19 |
| | TTCA | | T*+C*+A | | |
| 1253 | GCTGCCTATTCACAA | A571251Hi | +G*+C*+T*G*C*C*T*A*T*T*C*A*C*A*A*C*+ | 30289 | 19 |
| | CTTC | | T*+T*+C | | |
| 1254 | GCTGCCTATTCACAA | A571252Hi | +G*+C*+T*G*C*C*T*A*T*T*C*A*C*A*A*+C* | 30290 | 18 |
| | CTT | | +T*+T | | |
| 1255 | GGCTGCCTATTCACA | A571253Hi | +G*+G*+C*T*G*C*C*T*A*T*T*C*A*C*A*+A* | 30291 | 18 |
| | ACT | | +C*+T | | |
| 1256 | TGGCTGCCTATTCAC | A571254Hi | +T*+G*+G*C*T*G*C*C*T*A*T*T*C*A*C*+A* | 30292 | 18 |
| | AAC | | +A*+C | | |
| 1257 | CCTGGCTGCCTATTC | A571255Hi | +C*+C*+T*G*G*C*T*G*C*C*T*A*T*T*C*A*+ | 30293 | 19 |
| | ACAA | | C*+A*+A | | |
| 1258 | CCTGGCTGCCTATTC | A571256Hi | +C*+C*+T*G*G*C*T*G*C*C*T*A*T*T*C*+A* | 30294 | 18 |
| | ACA | | +C*+A | | |
| 1259 | TCCTGGCTGCCTATT | A571257Hi | +T*+C*+C*T*G*G*C*T*G*C*C*T*A*T*T*C*+ | 30294 | 19 |
| | CACA | | A*+C*+A | | |
| 1260 | TTCCTGGCTGCCTAT | A571258Hi | +T*+T*+C*C*T*G*G*C*T*G*C*C*T*A*T*T*+ | 30295 | 19 |
| | TCAC | | C*+A*+C | | |
| 1261 | TCCTGGCTGCCTATT | A571259Hi | +T*+C*+C*T*G*G*C*T*G*C*C*T*A*T*T*+C* | 30295 | 18 |
| | CAC | | +A*+C | | |
| 1262 | TTTCCTGGCTGCCTA | A571260Hi | +T*+T*+T*C*C*T*G*G*C*T*G*C*C*T*A*T*+ | 30296 | 19 |
| | TTCA | | T*+C*+A | | |
| 1263 | TTCCTGGCTGCCTAT | A571261Hi | +T*+T*+C*C*T*G*G*C*T*G*C*C*T*A*T*+T* | 30296 | 18 |
| | TCA | | +C*+A | | |
| 1264 | TCTTTCCTGGCTGCC | A571262Hi | +T*+C*+T*T*T*C*C*T*G*G*C*T*G*C*C*T*+ | 30298 | 19 |
| | TATT | | A*+T*+T | | |
| 1265 | TCTTTCCTGGCTGCC | A571263Hi | +T*+C*+T*T*T*C*C*T*G*G*C*T*G*C*C*+T* | 30299 | 18 |
| | TAT | | +A*+T | | |
| 1266 | TTCTTTCCTGGCTGC | A571264Hi | +T*+T*+C*T*T*T*C*C*T*G*G*C*T*G*C*C*+ | 30299 | 19 |
| | CTAT | | T*+A*+T | | |
| 1267 | CTTCTTTCCTGGCTG | A571265Hi | +C*+T*+T*C*T*T*T*C*C*T*G*G*C*T*G*C*+ | 30300 | 19 |
| | CCTA | | C*+T*+A | | |
| 1268 | TGCAGGTCTTACTCA | A571266Hi | +T*+G*+C*A*G*G*T*C*T*T*A*C*T*C*A*A*+ | 30323 | 19 |
| | ATAG | | T*+A*+G | | |
| 1269 | TGCAGGTCTTACTCA | A571267Hi | +T*+G*+C*A*G*G*T*C*T*T*A*C*T*C*A*+A* | 30324 | 18 |
| | ATA | | +T*+A | | |
| 1270 | AATTTCATCATAAAG | A571268Hi | +A*+A*+T*T*T*C*A*T*C*A*T*A*A*A*G*T*+ | 30472 | 19 |
| | TCAC | | C*+A*+C | | |
| 1271 | TGCAAGCAATAAACC | A571269Hi | +T*+G*+C*A*A*G*C*A*A*T*A*A*A*C*C*+T* | 30498 | 18 |
| | TAG | | +A*+G | | |
| 1272 | TTGCAAGCAATAAAC | A571270Hi | +T*+T*+G*C*A*A*G*C*A*A*T*A*A*A*C*C*+ | 30498 | 19 |
| | CTAG | | T*+A*+G | | |
| 1273 | ACTATCCTAGACAGT | A571271Hi | +A*+C*+T*A*T*C*C*T*A*G*A*C*A*G*T*+A* | 30536 | 18 |
| | AAC | | +A*+C | | |
| 1274 | CTTTAAGAATCACTA | A571272Hi | +C*+T*+T*T*A*A*G*A*A*T*C*A*C*T*A*T*+ | 30546 | 19 |
| | TCCT | | C*+C*+T | | |
| 1275 | AGTGGCTAGCTTCTA | A571273Hi | +A*+G*+T*G*G*C*T*A*G*C*T*T*C*T*A*G*+ | 30770 | 19 |
| | GCCA | | C*+C*+A | | |
| 1276 | GTGGCTAGCTTCTAG | A571274Hi | +G*+T*+G*G*C*T*A*G*C*T*T*C*T*A*G*+C* | 30770 | 18 |
| | CCA | | +C*+A | | |
| 1277 | AAAGTGGCTAGCTTC | A571275Hi | +A*+A*+A*G*T*G*G*C*T*A*G*C*T*T*C*T*+ | 30772 | 19 |
| | TAGC | | A*+G*+C | | |
| 1278 | CAAAGTGGCTAGCTT | A571276Hi | +C*+A*+A*A*G*T*G*G*C*T*A*G*C*T*T*C*+ | 30773 | 19 |
| | CTAG | | T*+A*+G | | |
| 1279 | AACACTTTGAAAGTG | A571277Hi | +A*+A*+C*A*C*T*T*T*G*A*A*A*G*T*G*G*+ | 31023 | 19 |
| | GCAG | | C*+A*+G | | |
| 1280 | GTGATTCAACACTTT | A571278Hi | +G*+T*+G*A*T*T*C*A*A*C*A*C*T*T*T*G*+ | 31030 | 19 |
| | GAAA | | A*+A*+A | | |
| 1281 | CACTGTGATTCAACA | A571279Hi | +C*+A*+C*T*G*T*G*A*T*T*C*A*A*C*A*C*+ | 31034 | 19 |
| | CTTT | | T*+T*+T | | |
| 1282 | TACACACTGTGATTC | A571280Hi | +T*+A*+C*A*C*A*C*T*G*T*G*A*T*T*C*A*+ | 31038 | 19 |
| | AACA | | A*+C*+A | | |
| 1283 | CCAACATTACACACT | A571281Hi | +C*+C*+A*A*C*A*T*T*A*C*A*C*A*C*T*+G* | 31046 | 18 |
| | GTG | | +T*+G | | |
| 1284 | ATCAATTGTCTTGAA | A571282Hi | +A*+T*+C*A*A*T*T*G*T*C*T*T*G*A*A*+G* | 31082 | 18 |
| | GCC | | +C*+C | | |
| 1285 | GTCTTCAAGTTTCCA | A571283Hi | +G*+T*+C*T*T*C*A*A*G*T*T*T*C*C*A*C*+ | 31103 | 19 |
| | CATA | | A*+T*+A | | |
| 1286 | TGTCTTCAAGTTTCC | A571284Hi | +T*+G*+T*C*T*T*C*A*A*G*T*T*T*C*C*A*+ | 31104 | 19 |
| | ACAT | | C*+A*+T | | |
| 1287 | AACTTCCTTCACTAA | A571285Hi | +A*+A*+C*T*T*C*C*T*T*C*A*C*T*A*A*A*+ | 31131 | 19 |
| | ACAT | | C*+A*+T | | |
| 1288 | ATAAACTTCCTTCACT | A571286Hi | +A*+T*+A*A*A*C*T*T*C*C*T*T*C*A*C*T*+ | 31134 | 19 |
| | AAA | | A*+A*+A | | |
| 1289 | CATTAACTCCAAAAC | A571287H | +C*+A*+T*T*A*A*C*T*C*C*A*A*A*A*C*A*+ | 32292 | 19 |
| | ATTC | | T*+T*+C | | |
| 1290 | TGCATTAACTCCAAA | A571288H | +T*+G*+C*A*T*T*A*A*C*T*C*C*A*A*A*A*+ | 32294 | 19 |
| | ACAT | | C*+A*+T | | |
| 1291 | TCTGCATTAACTCCA | A571289H | +T*+C*+T*G*C*A*T*T*A*A*C*T*C*C*A*A*+ | 32296 | 19 |
| | AAAC | | A*+A*+C | | |
| 1292 | TTCTGCATTAACTCC | A571290H | +T*+T*+C*T*G*C*A*T*T*A*A*C*T*C*C*A*+ | 32297 | 19 |
| | AAAA | | A*+A*+A | | |
| 1293 | ATGCTTCTGCATTAA | A571291H | +A*+T*+G*C*T*T*C*T*G*C*A*T*T*A*A*C*+ | 32301 | 19 |
| | CTCC | | T*+C*+C | | |
| 1294 | GTATATGCTTCTGCA | A571292H | +G*+T*+A*T*A*T*G*C*T*T*C*T*G*C*A*T*+ | 32305 | 19 |
| | TTAA | | T*+A*+A | | |
| 1295 | ATGTATATGCTTCTG | A571293H | +A*+T*+G*T*A*T*A*T*G*C*T*T*C*T*G*C*+ | 32307 | 19 |
| | CATT | | A*+T*+T | | |
| 1296 | AATGTATATGCTTCT | A571294H | +A*+A*+T*G*T*A*T*A*T*G*C*T*T*C*T*G*+ | 32308 | 19 |
| | GCAT | | C*+A*+T | | |
| 1297 | GGAGAATGTATATGC | A571295H | +G*+G*+A*G*A*A*T*G*T*A*T*A*T*G*C*T*+ | 32312 | 19 |
| | TTCT | | T*+C*+T | | |
| 1298 | CAGCCACTTCAGGAG | A571296H | +C*+A*+G*C*C*A*C*T*T*C*A*G*G*A*G*A*+ | 32323 | 19 |
| | AATG | | A*+T*+G | | |
| 1299 | CACTTTCAGCCACTT | A571297H | +C*+A*+C*T*T*T*C*A*G*C*C*A*C*T*T*C*+ | 32329 | 19 |
| | CAGG | | A*+G*+G | | |
| 1300 | CCACTTTCAGCCACT | A571298H | +C*+C*+A*C*T*T*T*C*A*G*C*C*A*C*T*T*+ | 32330 | 19 |
| | TCAG | | C*+A*+G | | |
| 1301 | GCCACTTTCAGCCAC | A571299H | +G*+C*+C*A*C*T*T*T*C*A*G*C*C*A*C*T*+ | 32331 | 19 |
| | TTCA | | T*+C*+A | | |
| 1302 | GATTTAGCATTTTAC | A571300H | +G*+A*+T*T*T*A*G*C*A*T*T*T*T*A*C*A*+ | 32347 | 19 |
| | AGCC | | G*+C*+C | | |
| 1303 | AGATGATTTAGCATT | A571301H | +A*+G*+A*T*G*A*T*T*T*A*G*C*A*T*T*T*+ | 32351 | 19 |
| | TTAC | | T*+A*+C | | |
| 1304 | CCTTCAAAAAGATGA | A571302H | +C*+C*+T*T*C*A*A*A*A*A*G*A*T*G*A*T*+ | 32360 | 19 |
| | TTTA | | T*+T*+A | | |
| 1305 | CTTCCTTCAAAAAGA | A571303H | +C*+T*+T*C*C*T*T*C*A*A*A*A*A*G*A*T*+ | 32363 | 19 |
| | TGAT | | G*+A*+T | | |
| 1306 | TTCTGTACAAGTTAA | A571304Hi | +T*+T*+C*T*G*T*A*C*A*A*G*T*T*A*A*T*+ | 32392 | 19 |
| | TGAA | | G*+A*+A | | |
| 1307 | CCAATATGATATATT | A571305Hi | +C*+C*+A*A*T*A*T*G*A*T*A*T*A*T*T*C*+ | 32405 | 19 |
| | CTGT | | T*+G*+T | | |
| 1308 | TTGATATTCTACCTG | A571306Hi | +T*+T*+G*A*T*A*T*T*C*T*A*C*C*T*G*+G* | 32423 | 18 |
| | GCC | | +C*+C | | |
| 1309 | TATTGATATTCTACCT | A571307Hi | +T*+A*+T*T*G*A*T*A*T*T*C*T*A*C*C*T*+ | 32424 | 19 |
| | GGC | | G*+G*+C | | |
| 1310 | ATTGATATTCTACCT | A571308Hi | +A*+T*+T*G*A*T*A*T*T*C*T*A*C*C*T*+G* | 32424 | 18 |
| | GGC | | +G*+C | | |
| 1311 | GTTAGAAATATGCTT | A571309Hi | +G*+T*+T*A*G*A*A*A*T*A*T*G*C*T*T*G*+ | 32443 | 19 |
| | GAAA | | A*+A*+A | | |
| 1312 | ACACGAGATTACATT | A571310Hi | +A*+C*+A*C*G*A*G*A*T*T*A*C*A*T*T*+G* | 32510 | 18 |
| | GCA | | +C*+A | | |
| 1313 | ACACGAGATTACATT | A571311Hi | +A*+C*+A*C*G*A*G*A*T*T*A*C*A*T*+T*+G | 32511 | 17 |
| | GC | | *+C | | |
| 1314 | TACTAGTGACACGAG | A571312Hi | +T*+A*+C*T*A*G*T*G*A*C*A*C*G*A*G*+A* | 32518 | 18 |
| | ATT | | +T*+T | | |
| 1315 | ACTAGTGACACGAGA | A571313Hi | +A*+C*+T*A*G*T*G*A*C*A*C*G*A*G*+A*+T | 32518 | 17 |
| | TT | | *+T | | |
| 1316 | TACTAGTGACACGAG | A571314Hi | +T*+A*+C*T*A*G*T*G*A*C*A*C*G*A*+G*+A | 32519 | 17 |
| | AT | | *+T | | |
| 1317 | GTACTAGTGACACGA | A571315Hi | +G*+T*+A*C*T*A*G*T*G*A*C*A*C*G*+A*+ | 32520 | 17 |
| | GA | | G*+A | | |
| 1318 | GGTACTAGTGACACG | A571316Hi | +G*+G*+T*A*C*T*A*G*T*G*A*C*A*C*+G*+ | 32521 | 17 |
| | AG | | A*+G | | |
| 1319 | TGGTACTAGTGACAC | A571317Hi | +T*+G*+G*T*A*C*T*A*G*T*G*A*C*A*+C*+G | 32522 | 17 |
| | GA | | *+A | | |
| 1320 | AAGTCTATGGTACTA | A571318Hi | +A*+A*+G*T*C*T*A*T*G*G*T*A*C*T*A*+G* | 32528 | 18 |
| | GTG | | +T*+G | | |
| 1321 | TCAGTGTTCAGATAA | A571319Hi | +T*+C*+A*G*T*G*T*T*C*A*G*A*T*A*A*A*+ | 32545 | 19 |
| | AGTA | | G*+T*+A | | |
| 1322 | ATCTTGCCATTCCTTT | A571320Hi | +A*+T*+C*T*T*G*C*C*A*T*T*C*C*T*T*T*+ | 32560 | 19 |
| | CAG | | C*+A*+G | | |
| 1323 | CCACAATCTTGCCAT | A571321Hi | +C*+C*+A*C*A*A*T*C*T*T*G*C*C*A*T*T*+ | 32565 | 19 |
| | TCCT | | C*+C*+T | | |
| 1324 | CACAATCTTGCCATT | A571322Hi | +C*+A*+C*A*A*T*C*T*T*G*C*C*A*T*T*+C* | 32565 | 18 |
| | CCT | | +C*+T | | |
| 1325 | TCCACAATCTTGCCA | A571323Hi | +T*+C*+C*A*C*A*A*T*C*T*T*G*C*C*A*T*+ | 32566 | 19 |
| | TTCC | | T*+C*+C | | |
| 1326 | TGTTTCCACAATCTT | A571324Hi | +T*+G*+T*T*T*C*C*A*C*A*A*T*C*T*T*G*+ | 32570 | 19 |
| | GCCA | | C*+C*+A | | |
| 1327 | ATGTTTCCACAATCTT | A571325Hi | +A*+T*+G*T*T*T*C*C*A*C*A*A*T*C*T*T*+ | 32571 | 19 |
| | GCC | | G*+C*+C | | |
| 1328 | TTCAACATGTTTCCA | A571326Hi | +T*+T*+C*A*A*C*A*T*G*T*T*T*C*C*A*C*+ | 32577 | 19 |
| | CAAT | | A*+A*+T | | |
| 1329 | ACCTTCAACATGTTT | A571327Hi | +A*+C*+C*T*T*C*A*A*C*A*T*G*T*T*T*C*+ | 32580 | 19 |
| | CCAC | | C*+A*+C | | |
| 1330 | CAACATCAAGCATCA | A571328Hi | +C*+A*+A*C*A*T*C*A*A*G*C*A*T*C*A*G*+ | 32608 | 19 |
| | GGTT | | G*+T*+T | | |
| 1331 | TGCTATACAATTACC | A571329Hi | +T*+G*+C*T*A*T*A*C*A*A*T*T*A*C*C*+A* | 32641 | 18 |
| | ACT | | +C*+T | | |
| 1332 | ATATTCGTTGTGAAA | A571330Hi | +A*+T*+A*T*T*C*G*T*T*G*T*G*A*A*+A*+G | 32738 | 17 |
| | GA | | *+A | | |
| 1333 | TATATTCGTTGTGAA | A571331Hi | +T*+A*+T*A*T*T*C*G*T*T*G*T*G*A*A*+A* | 32738 | 18 |
| | AGA | | +G*+A | | |
| 1334 | GAATAATATATTCGT | A571332Hi | +G*+A*+A*T*A*A*T*A*T*A*T*T*C*G*T*T*+ | 32743 | 19 |
| | TGTG | | G*+T*+G | | |
| 1335 | CTTGGACTAGATGTA | A571333Hi | +C*+T*+T*G*G*A*C*T*A*G*A*T*G*T*A*+G* | 32823 | 18 |
| | GTT | | +T*+T | | |
| 1336 | ACTTGGACTAGATGT | A571334Hi | +A*+C*+T*T*G*G*A*C*T*A*G*A*T*G*T*A*+ | 32823 | 19 |
| | AGTT | | G*+T*+T | | |
| 1337 | ACTTGGACTAGATGT | A571335Hi | +A*+C*+T*T*G*G*A*C*T*A*G*A*T*G*T*+A* | 32824 | 18 |
| | AGT | | +G*+T | | |
| 1338 | GCACTTGGACTAGAT | A571336Hi | +G*+C*+A*C*T*T*G*G*A*C*T*A*G*A*T*+G* | 32826 | 18 |
| | GTA | | +T*+A | | |
| 1339 | TGGTCTCTGTTGTGC | A571337Hi | +T*+G*+G*T*C*T*C*T*G*T*T*G*T*G*C*C*+ | 32874 | 19 |
| | CACT | | A*+C*+T | | |
| 1340 | TGGTCTCTGTTGTGC | A571338Hi | +T*+G*+G*T*C*T*C*T*G*T*T*G*T*G*C*+C* | 32875 | 18 |
| | CAC | | +A*+C | | |
| 1341 | AGTTGGCTCAAATTA | A571339Hi | +A*+G*+T*T*G*G*C*T*C*A*A*A*T*T*A*G*+ | 33039 | 19 |
| | GTGG | | T*+G*+G | | |
| 1342 | GTTGGCTCAAATTAG | A571340Hi | +G*+T*+T*G*G*C*T*C*A*A*A*T*T*A*G*+T* | 33039 | 18 |
| | TGG | | +G*+G | | |
| 1343 | AGTTGGCTCAAATTA | A571341Hi | +A*+G*+T*T*G*G*C*T*C*A*A*A*T*T*A*+G* | 33040 | 18 |
| | GTG | | +T*+G | | |
| 1344 | GATGAAACAGTTGGC | A571342Hi | +G*+A*+T*G*A*A*A*C*A*G*T*T*G*G*C*T*+ | 33047 | 19 |
| | TCAA | | C*+A*+A | | |
| 1345 | GAGATGAAACAGTTG | A571343Hi | +G*+A*+G*A*T*G*A*A*A*C*A*G*T*T*G*+G* | 33050 | 18 |
| | GCT | | +C*+T | | |
| 1346 | TAGAGATGAAACAGT | A571344Hi | +T*+A*+G*A*G*A*T*G*A*A*A*C*A*G*T*T*+ | 33051 | 19 |
| | TGGC | | G*+G*+C | | |
| 1347 | AGAGATGAAACAGTT | A571345Hi | +A*+G*+A*G*A*T*G*A*A*A*C*A*G*T*T*+G* | 33051 | 18 |
| | GGC | | +G*+C | | |
| 1348 | TACAACTTTCAAAGT | A571346Hi | +T*+A*+C*A*A*C*T*T*T*C*A*A*A*G*T*T*+ | 33108 | 19 |
| | TGTT | | G*+T*+T | | |
| 1349 | TACATTCTAAGCTTA | A571347Hi | +T*+A*+C*A*T*T*C*T*A*A*G*C*T*T*A*C*+ | 33121 | 19 |
| | CAAC | | A*+A*+C | | |
| 1350 | TTTAATCCTTACATTC | A571348Hi | +T*+T*+T*A*A*T*C*C*T*T*A*C*A*T*T*C*+ | 33130 | 19 |
| | TAA | | T*+A*+A | | |
| 1351 | GCAGGCATTATCCTA | A571349Hi | +G*+C*+A*G*G*C*A*T*T*A*T*C*C*T*A*A*+ | 33168 | 19 |
| | ATCC | | T*+C*+C | | |
| 1352 | ACTTCTTACAAGTCT | A571350Hi | +A*+C*+T*T*C*T*T*A*C*A*A*G*T*C*T*G*+ | 33281 | 19 |
| | GCAG | | C*+A*+G | | |
| 1353 | CTACTTCTTACAAGT | A571351Hi | +C*+T*+A*C*T*T*C*T*T*A*C*A*A*G*T*C*+ | 33283 | 19 |
| | CTGC | | T*+G*+C | | |
| 1354 | CTAGACTGGAGTTTA | A571352Hi | +C*+T*+A*G*A*C*T*G*G*A*G*T*T*T*A*T*+ | 33476 | 19 |
| | TTCC | | T*+C*+C | | |
| 1355 | TCCTAGACTGGAGTT | A571353Hi | +T*+C*+C*T*A*G*A*C*T*G*G*A*G*T*T*T*+ | 33478 | 19 |
| | TATT | | A*+T*+T | | |
| 1356 | CCTAGACTGGAGTTT | A571354Hi | +C*+C*+T*A*G*A*C*T*G*G*A*G*T*T*T*+A* | 33478 | 18 |
| | ATT | | +T*+T | | |
| 1357 | ATCCTAGACTGGAGT | A571355Hi | +A*+T*+C*C*T*A*G*A*C*T*G*G*A*G*T*+T* | 33480 | 18 |
| | TTA | | +T*+A | | |
| 1358 | AATCCTAGACTGGAG | A571356Hi | +A*+A*+T*C*C*T*A*G*A*C*T*G*G*A*G*+T* | 33481 | 18 |
| | TTT | | +T*+T | | |
| 1359 | GAATCCTAGACTGGA | A571357Hi | +G*+A*+A*T*C*C*T*A*G*A*C*T*G*G*A*G*+ | 33481 | 19 |
| | GTTT | | T*+T*+T | | |
| 1360 | TCTACAGAATCCTAG | A571358Hi | +T*+C*+T*A*C*A*G*A*A*T*C*C*T*A*G*A*+ | 33487 | 19 |
| | ACTG | | C*+T*+G | | |
| 1361 | CAGAATCTACAGAAT | A571359Hi | +C*+A*+G*A*A*T*C*T*A*C*A*G*A*A*T*C*+ | 33492 | 19 |
| | CCTA | | C*+T*+A | | |
| 1362 | CCAGAATCTACAGAA | A571360Hi | +C*+C*+A*G*A*A*T*C*T*A*C*A*G*A*A*T*+ | 33493 | 19 |
| | TCCT | | C*+C*+T | | |
| 1363 | CCAGAATCTACAGAA | A571361Hi | +C*+C*+A*G*A*A*T*C*T*A*C*A*G*A*A*+T* | 33494 | 18 |
| | TCC | | +C*+C | | |
| 1364 | TCTGAGACTGTATTA | A571362Hi | +T*+C*+T*G*A*G*A*C*T*G*T*A*T*T*A*T*+ | 33653 | 19 |
| | TAAT | | A*+A*+T | | |
| 1365 | TCTGTGGACACTGAG | A571363Hi | +T*+C*+T*G*T*G*G*A*C*A*C*T*G*A*G*A*+ | 33803 | 19 |
| | ATAC | | T*+A*+C | | |
| 1366 | GGTTCTGTGGACACT | A571364Hi | +G*+G*+T*T*C*T*G*T*G*G*A*C*A*C*T*G*+ | 33806 | 19 |
| | GAGA | | A*+G*+A | | |
| 1367 | TGGTTCTGTGGACAC | A571365Hi | +T*+G*+G*T*T*C*T*G*T*G*G*A*C*A*C*T*+ | 33807 | 19 |
| | TGAG | | G*+A*+G | | |
| 1368 | TTTGCATGGTTCTGT | A571366Hi | +T*+T*+T*G*C*A*T*G*G*T*T*C*T*G*T*G*+ | 33813 | 19 |
| | GGAC | | G*+A*+C | | |
| 1369 | CTTTGCATGGTTCTG | A571367Hi | +C*+T*+T*T*G*C*A*T*G*G*T*T*C*T*G*T*+ | 33814 | 19 |
| | TGGA | | G*+G*+A | | |
| 1370 | ACTTTGCATGGTTCT | A571368Hi | +A*+C*+T*T*T*G*C*A*T*G*G*T*T*C*T*G*+ | 33815 | 19 |
| | GTGG | | T*+G*+G | | |
| 1371 | GCATATCAGAACCAT | A571369Hi | +G*+C*+A*T*A*T*C*A*G*A*A*C*C*A*T*+G* | 33837 | 18 |
| | GTT | | +T*+T | | |
| 1372 | TGAACTTTAATACAG | A571370Hi | +T*+G*+A*A*C*T*T*T*A*A*T*A*C*A*G*T*+ | 33885 | 19 |
| | TCAA | | C*+A*+A | | |
| 1373 | ATGAACTTTAATACA | A571371Hi | +A*+T*+G*A*A*C*T*T*T*A*A*T*A*C*A*G*+ | 33886 | 19 |
| | GTCA | | T*+C*+A | | |
| 1374 | ATGAACTTTAATACA | A571372Hi | +A*+T*+G*A*A*C*T*T*T*A*A*T*A*C*A*+G* | 33887 | 18 |
| | GTC | | +T*+C | | |
| 1375 | TACTATGAACTTTAAT | A571373Hi | +T*+A*+C*T*A*T*G*A*A*C*T*T*T*A*A*T*+ | 33890 | 19 |
| | ACA | | A*+C*+A | | |
| 1376 | GTACTATGAACTTTA | A571374Hi | +G*+T*+A*C*T*A*T*G*A*A*C*T*T*T*A*A*+ | 33891 | 19 |
| | ATAC | | T*+A*+C | | |
| 1377 | GTAGGTACTATGAAC | A571375Hi | +G*+T*+A*G*G*T*A*C*T*A*T*G*A*A*C*T*+ | 33895 | 19 |
| | TTTA | | T*+T*+A | | |
| 1378 | TAGGTACTATGAACT | A571376Hi | +T*+A*+G*G*T*A*C*T*A*T*G*A*A*C*T*+T* | 33895 | 18 |
| | TTA | | +T*+A | | |
| 1379 | GCAAATGTCACTTGA | A571377Hi | +G*+C*+A*A*A*T*G*T*C*A*C*T*T*G*A*C*+ | 33920 | 19 |
| | CTAG | | T*+A*+G | | |
| 1380 | AGCAAATGTCACTTG | A571378Hi | +A*+G*+C*A*A*A*T*G*T*C*A*C*T*T*G*A*+ | 33921 | 19 |
| | ACTA | | C*+T*+A | | |
| 1381 | TGAGCAAATGTCACT | A571379Hi | +T*+G*+A*G*C*A*A*A*T*G*T*C*A*C*T*T*+ | 33923 | 19 |
| | TGAC | | G*+A*+C | | |
| 1382 | AGGTATTTAAGCTAT | A571380Hi | +A*+G*+G*T*A*T*T*T*A*A*G*C*T*A*T*T*+ | 33953 | 19 |
| | TCTT | | C*+T*+T | | |
| 1383 | TTAACATCACTGGAC | A571381Hi | +T*+T*+A*A*C*A*T*C*A*C*T*G*G*A*C*T*+ | 34024 | 19 |
| | TTCT | | T*+C*+T | | |
| 1384 | CAGATTAACATCACT | A571382Hi | +C*+A*+G*A*T*T*A*A*C*A*T*C*A*C*T*+G* | 34029 | 18 |
| | GGA | | +G*+A | | |
| 1385 | AAAGTGACAGAGACA | A571383Hi | +A*+A*+A*G*T*G*A*C*A*G*A*G*A*C*A*A*+ | 34110 | 19 |
| | AGGC | | G*+G*+C | | |
| 1386 | TAGAGGTTAAATATT | A571384Hi | +T*+A*+G*A*G*G*T*T*A*A*A*T*A*T*T*C*+ | 34141 | 19 |
| | CAGG | | A*+G*+G | | |
| 1387 | ATAGAGGTTAAATAT | A571385Hi | +A*+T*+A*G*A*G*G*T*T*A*A*A*T*A*T*T*+ | 34142 | 19 |
| | TCAG | | C*+A*+G | | |
| 1388 | AAACTTTGAACTCAT | A571386Hi | +A*+A*+A*C*T*T*T*G*A*A*C*T*C*A*T*A*+ | 34297 | 19 |
| | AAGA | | A*+G*+A | | |
| 1389 | TGGTATAAACACCAA | A571387Hi | +T*+G*+G*T*A*T*A*A*A*C*A*C*C*A*A*A*+ | 34417 | 19 |
| | ATTG | | T*+T*+G | | |
| 1390 | ATGGTATAAACACCA | A571388Hi | +A*+T*+G*G*T*A*T*A*A*A*C*A*C*C*A*A*+ | 34418 | 19 |
| | AATT | | A*+T*+T | | |
| 1391 | CTGATTCAATGGTAT | A571389Hi | +C*+T*+G*A*T*T*C*A*A*T*G*G*T*A*T*+A* | 34427 | 18 |
| | AAA | | +A*+A | | |
| 1392 | GTAATGATGTCTCAT | A571390H | +G*+T*+A*A*T*G*A*T*G*T*C*T*C*A*T*+G* | 34543 | 18 |
| | GAC | | +A*+C | | |
| 1393 | GGTAATGATGTCTCA | A571391H | +G*+G*+T*A*A*T*G*A*T*G*T*C*T*C*A*T*+ | 34543 | 19 |
| | TGAC | | G*+A*+C | | |
| 1394 | CTGGTAATGATGTCT | A571392H | +C*+T*+G*G*T*A*A*T*G*A*T*G*T*C*T*C*+ | 34545 | 19 |
| | CATG | | A*+T*+G | | |
| 1395 | CTGGTAATGATGTCT | A571393H | +C*+T*+G*G*T*A*A*T*G*A*T*G*T*C*T*+C* | 34546 | 18 |
| | CAT | | +A*+T | | |
| 1396 | ACTGGTAATGATGTC | A571394H | +A*+C*+T*G*G*T*A*A*T*G*A*T*G*T*C*T*+ | 34546 | 19 |
| | TCAT | | C*+A*+T | | |
| 1397 | GCACTGGTAATGATG | A571395H | +G*+C*+A*C*T*G*G*T*A*A*T*G*A*T*G*T*+ | 34548 | 19 |
| | TCTC | | C*+T*+C | | |
| 1398 | CTGCACTGGTAATGA | A571396H | +C*+T*+G*C*A*C*T*G*G*T*A*A*T*G*A*T*+ | 34550 | 19 |
| | TGTC | | G*+T*+C | | |
| 1399 | GCTGCACTGGTAATG | A571397H | +G*+C*+T*G*C*A*C*T*G*G*T*A*A*T*G*A*+ | 34551 | 19 |
| | ATGT | | T*+G*+T | | |
| 1400 | TCCAGCTGCACTGGT | A571398H | +T*+C*+C*A*G*C*T*G*C*A*C*T*G*G*T*A*+ | 34555 | 19 |
| | AATG | | A*+T*+G | | |
| 1401 | CCTCCAGCTGCACTG | A571399H | +C*+C*+T*C*C*A*G*C*T*G*C*A*C*T*G*G*+ | 34557 | 19 |
| | GTAA | | T*+A*+A | | |
| 1402 | GCCTCCAGCTGCACT | A571400H | +G*+C*+C*T*C*C*A*G*C*T*G*C*A*C*T*G*+ | 34558 | 19 |
| | GGTA | | G*+T*+A | | |
| 1403 | CGCCTCCAGCTGCAC | A571401H | +C*+G*+C*C*T*C*C*A*G*C*T*G*C*A*C*T*+ | 34559 | 19 |
| | TGGT | | G*+G*+T | | |
| 1404 | GCGCCTCCAGCTGCA | A571402H | +G*+C*+G*C*C*T*C*C*A*G*C*T*G*C*A*C*+ | 34560 | 19 |
| | CTGG | | T*+G*+G | | |
| 1405 | GAAGCGCCTCCAGCT | A571403H | +G*+A*+A*G*C*G*C*C*T*C*C*A*G*C*T*G*+ | 34563 | 19 |
| | GCAC | | C*+A*+C | | |
| 1406 | CGAAGCGCCTCCAGC | A571404H | +C*+G*+A*A*G*C*G*C*C*T*C*C*A*G*C*T*+ | 34564 | 19 |
| | TGCA | | G*+C*+A | | |
| 1407 | TCGAAGCGCCTCCAG | A571405H | +T*+C*+G*A*A*G*C*G*C*C*T*C*C*A*G*C*+ | 34565 | 19 |
| | CTGC | | T*+G*+C | | |
| 1408 | TCGAAGCGCCTCCAG | A571406H | +T*+C*+G*A*A*G*C*G*C*C*T*C*C*A*G*+C* | 34566 | 18 |
| | CTG | | +T*+G | | |
| 1409 | CTCGAAGCGCCTCCA | A571407H | +C*+T*+C*G*A*A*G*C*G*C*C*T*C*C*A*G*+ | 34566 | 19 |
| | GCTG | | C*+T*+G | | |
| 1410 | CTCGAAGCGCCTCCA | A571408H | +C*+T*+C*G*A*A*G*C*G*C*C*T*C*C*A*+G* | 34567 | 18 |
| | GCT | | +C*+T | | |
| 1411 | GCTCGAAGCGCCTCC | A571409H | +G*+C*+T*C*G*A*A*G*C*G*C*C*T*C*C*A*+ | 34567 | 19 |
| | AGCT | | G*+C*+T | | |
| 1412 | CTCGAAGCGCCTCCA | A571410H | +C*+T*+C*G*A*A*G*C*G*C*C*T*C*C*+A*+ | 34568 | 17 |
| | GC | | G*+C | | |
| 1413 | GCTCGAAGCGCCTCC | A571411H | +G*+C*+T*C*G*A*A*G*C*G*C*C*T*C*C*+A* | 34568 | 18 |
| | AGC | | +G*+C | | |
| 1414 | AGCTCGAAGCGCCTC | A571412H | +A*+G*+C*T*C*G*A*A*G*C*G*C*C*T*C*C*+ | 34568 | 19 |
| | CAGC | | A*+G*+C | | |
| 1415 | GCTCGAAGCGCCTCC | A571413H | +G*+C*+T*C*G*A*A*G*C*G*C*C*T*C*+C*+ | 34569 | 17 |
| | AG | | A*+G | | |
| 1416 | AGCTCGAAGCGCCTC | A571414H | +A*+G*+C*T*C*G*A*A*G*C*G*C*C*T*C*+C* | 34569 | 18 |
| | CAG | | +A*+G | | |
| 1417 | TAGCTCGAAGCGCCT | A571415H | +T*+A*+G*C*T*C*G*A*A*G*C*G*C*C*T*C*+ | 34569 | 19 |
| | CCAG | | C*+A*+G | | |
| 1418 | TAGCTCGAAGCGCCT | A571416H | +T*+A*+G*C*T*C*G*A*A*G*C*G*C*C*T*+C* | 34570 | 18 |
| | CCA | | +C*+A | | |
| 1419 | AGCTCGAAGCGCCTC | A571417H | +A*+G*+C*T*C*G*A*A*G*C*G*C*C*T*+C*+ | 34570 | 17 |
| | CA | | C*+A | | |
| 1420 | ATAGCTCGAAGCGCC | A571418H | +A*+T*+A*G*C*T*C*G*A*A*G*C*G*C*C*T*+ | 34570 | 19 |
| | TCCA | | C*+C*+A | | |
| 1421 | TAGCTCGAAGCGCCT | A571419H | +T*+A*+G*C*T*C*G*A*A*G*C*G*C*C*+T*+C | 34571 | 17 |
| | CC | | *+C | | |
| 1422 | ATAGCTCGAAGCGCC | A571420H | +A*+T*+A*G*C*T*C*G*A*A*G*C*G*C*C*+T* | 34571 | 18 |
| | TCC | | +C*+C | | |
| 1423 | AGCTCGAAGCGCCTC | A571421H | +A*+G*+C*T*C*G*A*A*G*C*G*C*C*+T*+C*+ | 34571 | 16 |
| | C | | C | | |
| 1424 | AATAGCTCGAAGCGC | A571422H | +A*+A*+T*A*G*C*T*C*G*A*A*G*C*G*C*C*+ | 34571 | 19 |
| | CTCC | | T*+C*+C | | |
| 1425 | AATAGCTCGAAGCGC | A571423H | +A*+A*+T*A*G*C*T*C*G*A*A*G*C*G*C*+C* | 34572 | 18 |
| | CTC | | +T*+C | | |
| 1426 | AAATAGCTCGAAGCG | A571424H | +A*+A*+A*T*A*G*C*T*C*G*A*A*G*C*G*C*+ | 34572 | 19 |
| | CCTC | | C*+T*+C | | |
| 1427 | AATAGCTCGAAGCGC | A571425H | +A*+A*+T*A*G*C*T*C*G*A*A*G*C*G*+C*+ | 34573 | 17 |
| | CT | | C*+T | | |
| 1428 | AAATAGCTCGAAGCG | A571426H | +A*+A*+A*T*A*G*C*T*C*G*A*A*G*C*G*+C* | 34573 | 18 |
| | CCT | | +C*+T | | |
| 1429 | AAAATAGCTCGAAGC | A571427H | +A*+A*+A*A*T*A*G*C*T*C*G*A*A*G*C*G*+ | 34573 | 19 |
| | GCCT | | C*+C*+T | | |
| 1430 | AATAGCTCGAAGCGC | A571428H | +A*+A*+T*A*G*C*T*C*G*A*A*G*C*+G*+C*+ | 34574 | 16 |
| | C | | C | | |
| 1431 | AAATAGCTCGAAGCG | A571429H | +A*+A*+A*T*A*G*C*T*C*G*A*A*G*C*+G*+ | 34574 | 17 |
| | CC | | C*+C | | |
| 1432 | AAAATAGCTCGAAGC | A571430H | +A*+A*+A*A*T*A*G*C*T*C*G*A*A*G*C*+G* | 34574 | 18 |
| | GCC | | +C*+C | | |
| 1433 | TAAAATAGCTCGAAG | A571431H | +T*+A*+A*A*A*T*A*G*C*T*C*G*A*A*G*C*+ | 34574 | 19 |
| | CGCC | | G*+C*+C | | |
| 1434 | AAAATAGCTCGAAGC | A571432H | +A*+A*+A*A*T*A*G*C*T*C*G*A*A*G*+C*+ | 34575 | 17 |
| | GC | | G*+C | | |
| 1435 | TAAAATAGCTCGAAG | A571433H | +T*+A*+A*A*A*T*A*G*C*T*C*G*A*A*G*+C* | 34575 | 18 |
| | CGC | | +G*+C | | |
| 1436 | GTAAAATAGCTCGAA | A571434H | +G*+T*+A*A*A*A*T*A*G*C*T*C*G*A*A*G*+ | 34575 | 19 |
| | GCGC | | C*+G*+C | | |
| 1437 | TAAAATAGCTCGAAG | A571435H | +T*+A*+A*A*A*T*A*G*C*T*C*G*A*A*+G*+C | 34576 | 17 |
| | CG | | *+G | | |
| 1438 | GTAAAATAGCTCGAA | A571436H | +G*+T*+A*A*A*A*T*A*G*C*T*C*G*A*A*+G* | 34576 | 18 |
| | GCG | | +C*+G | | |
| 1439 | TGTAAAATAGCTCGA | A571437H | +T*+G*+T*A*A*A*A*T*A*G*C*T*C*G*A*A*+ | 34576 | 19 |
| | AGCG | | G*+C*+G | | |
| 1440 | ATGTAAAATAGCTCG | A571438H | +A*+T*+G*T*A*A*A*A*T*A*G*C*T*C*G*A*+ | 34577 | 19 |
| | AAGC | | A*+G*+C | | |
| 1441 | CAATCAACTATGTAA | A571439Hi | +C*+A*+A*T*C*A*A*C*T*A*T*G*T*A*A*C*+ | 34610 | 19 |
| | CTTA | | T*+T*+A | | |
| 1442 | ACAATCAACTATGTA | A571440Hi | +A*+C*+A*A*T*C*A*A*C*T*A*T*G*T*A*A*+ | 34611 | 19 |
| | ACTT | | C*+T*+T | | |
| 1443 | TTAGCTAATAGTACA | A571441Hi | +T*+T*+A*G*C*T*A*A*T*A*G*T*A*C*A*A*+ | 34943 | 19 |
| | ATTC | | T*+T*+C | | |
| 1444 | TAGCTAATAGTACAA | A571442Hi | +T*+A*+G*C*T*A*A*T*A*G*T*A*C*A*A*+T* | 34943 | 18 |
| | TTC | | +T*+C | | |
| 1445 | AAGAAATTATTTGTG | A571443Hi | +A*+A*+G*A*A*A*T*T*A*T*T*T*G*T*G*C*+ | 35351 | 19 |
| | CCTC | | C*+T*+C | | |
| 1446 | CCTTCCACCTCAAAT | A571444Hi | +C*+C*+T*T*C*C*A*C*C*T*C*A*A*A*T*A*+ | 35392 | 19 |
| | ATTA | | T*+T*+A | | |
| 1447 | TCGGTGTGACACTAC | A571445Hi | +T*+C*+G*G*T*G*T*G*A*C*A*C*T*A*+C*+A | 35637 | 17 |
| | AA | | *+A | | |
| 1448 | CAACCATCTGTCCAA | A571446Hi | +C*+A*+A*C*C*A*T*C*T*G*T*C*C*A*A*T*+ | 35800 | 19 |
| | TCTG | | C*+T*+G | | |
| 1449 | TGATAGATTTAGGAA | A571447Hi | +T*+G*+A*T*A*G*A*T*T*T*A*G*G*A*A*G*+ | 35928 | 19 |
| | GATT | | A*+T*+T | | |
| 1450 | AATGATAGATTTAGG | A571448Hi | +A*+A*+T*G*A*T*A*G*A*T*T*T*A*G*G*A*+ | 35930 | 19 |
| | AAGA | | A*+G*+A | | |
| 1451 | GACTGAAGAAAGCTC | A571449Hi | +G*+A*+C*T*G*A*A*G*A*A*A*G*C*T*C*T*+ | 36017 | 19 |
| | TGTC | | G*+T*+C | | |
| 1452 | ATGACTGAAGAAAGC | A571450Hi | +A*+T*+G*A*C*T*G*A*A*G*A*A*A*G*C*T*+ | 36019 | 19 |
| | TCTG | | C*+T*+G | | |
| 1453 | CTGAAACTATGAATA | A571451Hi | +C*+T*+G*A*A*A*C*T*A*T*G*A*A*T*A*G*+ | 36103 | 19 |
| | GGAC | | G*+A*+C | | |
| 1454 | AAGGCTGTCAACTTT | A571452Hi | +A*+A*+G*G*C*T*G*T*C*A*A*C*T*T*T*A*+ | 36238 | 19 |
| | AAGG | | A*+G*+G | | |
| 1455 | AAGGCTGTCAACTTT | A571453Hi | +A*+A*+G*G*C*T*G*T*C*A*A*C*T*T*T*+A* | 36239 | 18 |
| | AAG | | +A*+G | | |
| 1456 | GAATAAGGCTGTCAA | A571454Hi | +G*+A*+A*T*A*A*G*G*C*T*G*T*C*A*A*C*+ | 36242 | 19 |
| | CTTT | | T*+T*+T | | |
| 1457 | AGTACAGTCCTCAAG | A571455Hi | +A*+G*+T*A*C*A*G*T*C*C*T*C*A*A*G*T*+ | 36462 | 19 |
| | TGAT | | G*+A*+T | | |
| 1458 | AGTACAGTCCTCAAG | A571456Hi | +A*+G*+T*A*C*A*G*T*C*C*T*C*A*A*G*+T* | 36463 | 18 |
| | TGA | | +G*+A | | |
| 1459 | TGTACTTACATACTG | A571457Hi | +T*+G*+T*A*C*T*T*A*C*A*T*A*C*T*G*A*+ | 36508 | 19 |
| | ATAT | | T*+A*+T | | |
| 1460 | AATGTACTTACATAC | A571458Hi | +A*+A*+T*G*T*A*C*T*T*A*C*A*T*A*C*T*+ | 36510 | 19 |
| | TGAT | | G*+A*+T | | |
| 1461 | GAATGTACTTACATA | A571459Hi | +G*+A*+A*T*G*T*A*C*T*T*A*C*A*T*A*C*+ | 36511 | 19 |
| | CTGA | | T*+G*+A | | |
| 1462 | AGAATGTACTTACAT | A571460Hi | +A*+G*+A*A*T*G*T*A*C*T*T*A*C*A*T*A*+ | 36512 | 19 |
| | ACTG | | C*+T*+G | | |
| 1463 | AAGAATGTACTTACA | A571461Hi | +A*+A*+G*A*A*T*G*T*A*C*T*T*A*C*A*T*+ | 36513 | 19 |
| | TACT | | A*+C*+T | | |
| 1464 | TAAATGATTAAGCCT | A571462Hi | +T*+A*+A*A*T*G*A*T*T*A*A*G*C*C*T*T*+ | 36539 | 19 |
| | TTAC | | T*+A*+C | | |
| 1465 | AGTAAATGATTAAGC | A571463Hi | +A*+G*+T*A*A*A*T*G*A*T*T*A*A*G*C*C*+ | 36541 | 19 |
| | CTTT | | T*+T*+T | | |
| 1466 | AGTAAATGATTAAGC | A571464Hi | +A*+G*+T*A*A*A*T*G*A*T*T*A*A*G*C*+C* | 36542 | 18 |
| | CTT | | +T*+T | | |
| 1467 | ACTGCACGGCATGGT | A571465Hi | +A*+C*+T*G*C*A*C*G*G*C*A*T*G*G*T*+A* | 36616 | 18 |
| | ACA | | +C*+A | | |
| 1468 | GGTACTGCACGGCAT | A571466Hi | +G*+G*+T*A*C*T*G*C*A*C*G*G*C*A*+T*+ | 36620 | 17 |
| | GG | | G*+G | | |
| 1469 | ATGAATACATGTACA | A571467Hi | +A*+T*+G*A*A*T*A*C*A*T*G*T*A*C*A*C*+ | 36746 | 19 |
| | CCTA | | C*+T*+A | | |
| 1470 | TGCAATATGCTATCC | A571468Hi | +T*+G*+C*A*A*T*A*T*G*C*T*A*T*C*C*+T* | 36843 | 18 |
| | TTA | | +T*+A | | |
| 1471 | TAGATGCAATATGCT | A571469Hi | +T*+A*+G*A*T*G*C*A*A*T*A*T*G*C*T*A*+ | 36846 | 19 |
| | ATCC | | T*+C*+C | | |
| 1472 | CACTAGATGCAATAT | A571470Hi | +C*+A*+C*T*A*G*A*T*G*C*A*A*T*A*T*+G* | 36850 | 18 |
| | GCT | | +C*+T | | |
| 1473 | ATATCAGGCCTGACC | A571471Hi | +A*+T*+A*T*C*A*G*G*C*C*T*G*A*C*C*+T* | 37398 | 18 |
| | TTA | | +T*+A | | |
| 1474 | GTTATATCAGGCCTG | A571472Hi | +G*+T*+T*A*T*A*T*C*A*G*G*C*C*T*G*+A* | 37401 | 18 |
| | ACC | | +C*+C | | |
| 1475 | CTGTTATATCAGGCC | A571473Hi | +C*+T*+G*T*T*A*T*A*T*C*A*G*G*C*C*T*+ | 37402 | 19 |
| | TGAC | | G*+A*+C | | |
| 1476 | GTGCTGTTATATCAG | A571474Hi | +G*+T*+G*C*T*G*T*T*A*T*A*T*C*A*G*G*+ | 37405 | 19 |
| | GCCT | | C*+C*+T | | |
| 1477 | GTGCTGTTATATCAG | A571475Hi | +G*+T*+G*C*T*G*T*T*A*T*A*T*C*A*G*+G* | 37406 | 18 |
| | GCC | | +C*+C | | |
| 1478 | TGGTGCTGTTATATC | A571476Hi | +T*+G*+G*T*G*C*T*G*T*T*A*T*A*T*C*+A* | 37408 | 18 |
| | AGG | | +G*+G | | |
| 1479 | CTGGTGCTGTTATAT | A571477Hi | +C*+T*+G*G*T*G*C*T*G*T*T*A*T*A*T*C*+ | 37408 | 19 |
| | CAGG | | A*+G*+G | | |
| 1480 | ATCTGGTGCTGTTAT | A571478Hi | +A*+T*+C*T*G*G*T*G*C*T*G*T*T*A*T*A*+ | 37410 | 19 |
| | ATCA | | T*+C*+A | | |
| 1481 | AAATCTGGTGCTGTT | A571479Hi | +A*+A*+A*T*C*T*G*G*T*G*C*T*G*T*T*A*+ | 37412 | 19 |
| | ATAT | | T*+A*+T | | |
| 1482 | TGCCTACTCTGGAGT | A571480Hi | +T*+G*+C*C*T*A*C*T*C*T*G*G*A*G*T*C*+ | 37444 | 19 |
| | CAGC | | A*+G*+C | | |
| 1483 | AATACAACAGTTGTT | A571481Hi | +A*+A*+T*A*C*A*A*C*A*G*T*T*G*T*T*C*+ | 37495 | 19 |
| | CAGG | | A*+G*+G | | |
| 1484 | AAATACAACAGTTGT | A571482Hi | +A*+A*+A*T*A*C*A*A*C*A*G*T*T*G*T*T*+ | 37496 | 19 |
| | TCAG | | C*+A*+G | | |
| 1485 | TAAATACAACAGTTG | A571483Hi | +T*+A*+A*A*T*A*C*A*A*C*A*G*T*T*G*T*+ | 37497 | 19 |
| | TTCA | | T*+C*+A | | |
| 1486 | TTAAATACAACAGTT | A571484Hi | +T*+T*+A*A*A*T*A*C*A*A*C*A*G*T*T*G*+ | 37498 | 19 |
| | GTTC | | T*+T*+C | | |
| 1487 | GCTTTAAATACAACA | A571485Hi | +G*+C*+T*T*T*A*A*A*T*A*C*A*A*C*A*G*+ | 37501 | 19 |
| | GTTG | | T*+T*+G | | |
| 1488 | AGACATAGTGCTTTA | A571486Hi | +A*+G*+A*C*A*T*A*G*T*G*C*T*T*T*A*A*+ | 37510 | 19 |
| | AATA | | A*+T*+A | | |
| 1489 | GACATAGTGCTTTAA | A571487Hi | +G*+A*+C*A*T*A*G*T*G*C*T*T*T*A*A*+A* | 37510 | 18 |
| | ATA | | +T*+A | | |
| 1490 | CTTAAGGATTCTAGA | A571488Hi | +C*+T*+T*A*A*G*G*A*T*T*C*T*A*G*A*C*+ | 37522 | 19 |
| | CATA | | A*+T*+A | | |
| 1491 | TTCTATCACTTGAGG | A571489Hi | +T*+T*+C*T*A*T*C*A*C*T*T*G*A*G*G*T*+ | 37647 | 19 |
| | TCCT | | C*+C*+T | | |
| 1492 | GTTCTATCACTTGAG | A571490Hi | +G*+T*+T*C*T*A*T*C*A*C*T*T*G*A*G*G*+ | 37648 | 19 |
| | GTCC | | T*+C*+C | | |
| 1493 | CTTTGTTCTATCACTT | A571491Hi | +C*+T*+T*T*G*T*T*C*T*A*T*C*A*C*T*+T*+ | 37653 | 18 |
| | GA | | G*+A | | |
| 1494 | CCAAATGAACATTCC | A571492Hi | +C*+C*+A*A*A*T*G*A*A*C*A*T*T*C*C*T*+ | 37686 | 19 |
| | TGTA | | G*+T*+A | | |
| 1495 | ACCAAATGAACATTC | A571493Hi | +A*+C*+C*A*A*A*T*G*A*A*C*A*T*T*C*C*+ | 37687 | 19 |
| | CTGT | | T*+G*+T | | |
| 1496 | TAAGACCAAATGAAC | A571494Hi | +T*+A*+A*G*A*C*C*A*A*A*T*G*A*A*C*A*+ | 37691 | 19 |
| | ATTC | | T*+T*+C | | |
| 1497 | AACATCATGCTACAT | A571495Hi | +A*+A*+C*A*T*C*A*T*G*C*T*A*C*A*T*G*+ | 37738 | 19 |
| | GTAA | | T*+A*+A | | |
| 1498 | AAACATCATGCTACA | A571496Hi | +A*+A*+A*C*A*T*C*A*T*G*C*T*A*C*A*T*+ | 37739 | 19 |
| | TGTA | | G*+T*+A | | |
| 1499 | AACTTCTGAGGCTGC | A571497Hi | +A*+A*+C*T*T*C*T*G*A*G*G*C*T*G*C*C*+ | 37803 | 19 |
| | CTAT | | T*+A*+T | | |
| 1500 | AACTTCTGAGGCTGC | A571498Hi | +A*+A*+C*T*T*C*T*G*A*G*G*C*T*G*C*+C* | 37804 | 18 |
| | CTA | | +T*+A | | |
| 1501 | AGAGGTTCTGACATT | A571499Hi | +A*+G*+A*G*G*T*T*C*T*G*A*C*A*T*T*+C* | 37893 | 18 |
| | CAC | | +A*+C | | |
| 1502 | CAAAGAGGTTCTGAC | A571500Hi | +C*+A*+A*A*G*A*G*G*T*T*C*T*G*A*C*+A* | 37896 | 18 |
| | ATT | | +T*+T | | |
| 1503 | CAACTTTCAGTCCTT | A571501Hi | +C*+A*+A*C*T*T*T*C*A*G*T*C*C*T*T*A*+ | 37925 | 19 |
| | ATTT | | T*+T*+T | | |
| 1504 | CTCCAACTTTCAGTC | A571502Hi | +C*+T*+C*C*A*A*C*T*T*T*C*A*G*T*C*C*+ | 37928 | 19 |
| | CTTA | | T*+T*+A | | |
| 1505 | GAAGGCAGCCTTGAT | A571503Hi | +G*+A*+A*G*G*C*A*G*C*C*T*T*G*A*T*A*+ | 37953 | 19 |
| | AACC | | A*+C*+C | | |
| 1506 | GAAGGCAGCCTTGAT | A571504Hi | +G*+A*+A*G*G*C*A*G*C*C*T*T*G*A*T*+A* | 37954 | 18 |
| | AAC | | +A*+C | | |
| 1507 | TCCAATTACATGCCA | A571505Hi | +T*+C*+C*A*A*T*T*A*C*A*T*G*C*C*A*A*+ | 38002 | 19 |
| | AGGA | | G*+G*+A | | |
| 1508 | AAGAGTAGCTGGAAT | A571506Hi | +A*+A*+G*A*G*T*A*G*C*T*G*G*A*A*T*A*+ | 38365 | 19 |
| | AATC | | A*+T*+C | | |
| 1509 | AGAGTAGCTGGAATA | A571507Hi | +A*+G*+A*G*T*A*G*C*T*G*G*A*A*T*A*+A* | 38365 | 18 |
| | ATC | | +T*+C | | |
| 1510 | GTGCTTTAAGATGCT | A571508Hi | +G*+T*+G*C*T*T*T*A*A*G*A*T*G*C*T*G*+ | 38472 | 19 |
| | GGAA | | G*+A*+A | | |
| 1511 | CTGTGCTTTAAGATG | A571509Hi | +C*+T*+G*T*G*C*T*T*T*A*A*G*A*T*G*C*+ | 38474 | 19 |
| | CTGG | | T*+G*+G | | |
| 1512 | GAGTAGGCTGTGCTT | A571510Hi | +G*+A*+G*T*A*G*G*C*T*G*T*G*C*T*T*T*+ | 38481 | 19 |
| | TAAG | | A*+A*+G | | |
| 1513 | GAGTAGGCTGTGCTT | A571511Hi | +G*+A*+G*T*A*G*G*C*T*G*T*G*C*T*T*+T* | 38482 | 18 |
| | TAA | | +A*+A | | |
| 1514 | TGTGAGTAGGCTGTG | A571512Hi | +T*+G*+T*G*A*G*T*A*G*G*C*T*G*T*G*C*+ | 38484 | 19 |
| | CTTT | | T*+T*+T | | |
| 1515 | GTGTGAGTAGGCTGT | A571513Hi | +G*+T*+G*T*G*A*G*T*A*G*G*C*T*G*T*G*+ | 38485 | 19 |
| | GCTT | | C*+T*+T | | |
| 1516 | GTGTGAGTAGGCTGT | A571514Hi | +G*+T*+G*T*G*A*G*T*A*G*G*C*T*G*T*+G* | 38486 | 18 |
| | GCT | | +C*+T | | |
| 1517 | GTAAGAATGCTTACT | A571515Hi | +G*+T*+A*A*G*A*A*T*G*C*T*T*A*C*T*G*+ | 38564 | 19 |
| | GAAT | | A*+A*+T | | |
| 1518 | GTGTAAGAATGCTTA | A571516Hi | +G*+T*+G*T*A*A*G*A*A*T*G*C*T*T*A*C*+ | 38566 | 19 |
| | CTGA | | T*+G*+A | | |
| 1519 | TTGAGTTGAACAGAC | A571517Hi | +T*+T*+G*A*G*T*T*G*A*A*C*A*G*A*C*+T* | 38728 | 18 |
| | TGT | | +G*+T | | |
| 1520 | ATTTGAGTTGAACAG | A571518Hi | +A*+T*+T*T*G*A*G*T*T*G*A*A*C*A*G*A*+ | 38729 | 19 |
| | ACTG | | C*+T*+G | | |
| 1521 | TAAACATTTGAGTTG | A571519Hi | +T*+A*+A*A*C*A*T*T*T*G*A*G*T*T*G*A*+ | 38734 | 19 |
| | AACA | | A*+C*+A | | |
| 1522 | ATAAACATTTGAGTT | A571520Hi | +A*+T*+A*A*A*C*A*T*T*T*G*A*G*T*T*G*+ | 38735 | 19 |
| | GAAC | | A*+A*+C | | |
| 1523 | CTGGATTCTTCTGGC | A571521H | +C*+T*+G*G*A*T*T*C*T*T*C*T*G*G*C*A*+ | 38793 | 19 |
| | ATGC | | T*+G*+C | | |
| 1524 | CACTGTTTTTTAACCA | A571522H | +C*+A*+C*T*G*T*T*T*T*T*T*A*A*C*C*A*+ | 38841 | 19 |
| | TAT | | T*+A*+T | | |
| 1525 | CATTCTTGAAACACT | A571523H | +C*+A*+T*T*C*T*T*G*A*A*A*C*A*C*T*G*+ | 38852 | 19 |
| | GTTT | | T*+T*+T | | |
| 1526 | TATCATTCTTGAAAC | A571524H | +T*+A*+T*C*A*T*T*C*T*T*G*A*A*A*C*A*+ | 38855 | 19 |
| | ACTG | | C*+T*+G | | |
| 1527 | AATATCATTCTTGAA | A571525H | +A*+A*+T*A*T*C*A*T*T*C*T*T*G*A*A*A*+ | 38857 | 19 |
| | ACAC | | C*+A*+C | | |
| 1528 | TGAATATCATTCTTG | A571526H | +T*+G*+A*A*T*A*T*C*A*T*T*C*T*T*G*A*+ | 38859 | 19 |
| | AAAC | | A*+A*+C | | |
| 1529 | TTTGTGAATATCATTC | A571527H | +T*+T*+T*G*T*G*A*A*T*A*T*C*A*T*T*C*+ | 38863 | 19 |
| | TTG | | T*+T*+G | | |
| 1530 | GTTTGTGAATATCAT | A571528H | +G*+T*+T*T*G*T*G*A*A*T*A*T*C*A*T*T*+ | 38864 | 19 |
| | TCTT | | C*+T*+T | | |
| 1531 | AGTTTGTGAATATCA | A571529H | +A*+G*+T*T*T*G*T*G*A*A*T*A*T*C*A*T*+ | 38865 | 19 |
| | TTCT | | T*+C*+T | | |
| 1532 | CAGTTTGTGAATATC | A571530H | +C*+A*+G*T*T*T*G*T*G*A*A*T*A*T*C*A*+ | 38866 | 19 |
| | ATTC | | T*+T*+C | | |
| 1533 | AGGACCAGTTTGTGA | A571531H | +A*+G*+G*A*C*C*A*G*T*T*T*G*T*G*A*A*+ | 38871 | 19 |
| | ATAT | | T*+A*+T | | |
| 1534 | TAGGACCAGTTTGTG | A571532H | +T*+A*+G*G*A*C*C*A*G*T*T*T*G*T*G*A*+ | 38872 | 19 |
| | AATA | | A*+T*+A | | |
| 1535 | GCTAGGACCAGTTTG | A571533H | +G*+C*+T*A*G*G*A*C*C*A*G*T*T*T*G*T*+ | 38874 | 19 |
| | TGAA | | G*+A*+A | | |
| 1536 | GCTAGGACCAGTTTG | A571534H | +G*+C*+T*A*G*G*A*C*C*A*G*T*T*T*G*+T* | 38875 | 18 |
| | TGA | | +G*+A | | |
| 1537 | TGCTAGGACCAGTTT | A571535H | +T*+G*+C*T*A*G*G*A*C*C*A*G*T*T*T*G*+ | 38875 | 19 |
| | GTGA | | T*+G*+A | | |
| 1538 | GCTAGGACCAGTTTG | A571536H | +G*+C*+T*A*G*G*A*C*C*A*G*T*T*T*+G*+T | 38876 | 17 |
| | TG | | *+G | | |
| 1539 | TGCTAGGACCAGTTT | A571537H | +T*+G*+C*T*A*G*G*A*C*C*A*G*T*T*T*+G* | 38876 | 18 |
| | GTG | | +T*+G | | |
| 1540 | CTGCTAGGACCAGTT | A571538H | +C*+T*+G*C*T*A*G*G*A*C*C*A*G*T*T*T*+ | 38876 | 19 |
| | TGTG | | G*+T*+G | | |
| 1541 | CTGCTAGGACCAGTT | A571539H | +C*+T*+G*C*T*A*G*G*A*C*C*A*G*T*T*+T* | 38877 | 18 |
| | TGT | | +G*+T | | |
| 1542 | GCTGCTAGGACCAGT | A571540H | +G*+C*+T*G*C*T*A*G*G*A*C*C*A*G*T*T*+ | 38877 | 19 |
| | TTGT | | T*+G*+T | | |
| 1543 | AGCTGCTAGGACCAG | A571541H | +A*+G*+C*T*G*C*T*A*G*G*A*C*C*A*G*T*+ | 38878 | 19 |
| | TTTG | | T*+T*+G | | |
| 1544 | AAGCTGCTAGGACCA | A571542H | +A*+A*+G*C*T*G*C*T*A*G*G*A*C*C*A*G*+ | 38879 | 19 |
| | GTTT | | T*+T*+T | | |
| 1545 | TCAAAGCTGCTAGGA | A571543H | +T*+C*+A*A*A*G*C*T*G*C*T*A*G*G*A*C*+ | 38882 | 19 |
| | CCAG | | C*+A*+G | | |
| 1546 | TCAAAGCTGCTAGGA | A571544H | +T*+C*+A*A*A*G*C*T*G*C*T*A*G*G*A*+C* | 38883 | 18 |
| | CCA | | +C*+A | | |
| 1547 | TTCAAAGCTGCTAGG | A571545H | +T*+T*+C*A*A*A*G*C*T*G*C*T*A*G*G*A*+ | 38883 | 19 |
| | ACCA | | C*+C*+A | | |
| 1548 | GTTCAAAGCTGCTAG | A571546H | +G*+T*+T*C*A*A*A*G*C*T*G*C*T*A*G*G*+ | 38884 | 19 |
| | GACC | | A*+C*+C | | |
| 1549 | GTTCAAAGCTGCTAG | A571547H | +G*+T*+T*C*A*A*A*G*C*T*G*C*T*A*G*+G* | 38885 | 18 |
| | GAC | | +A*+C | | |
| 1550 | TGTTCAAAGCTGCTA | A571548H | +T*+G*+T*T*C*A*A*A*G*C*T*G*C*T*A*G*+ | 38885 | 19 |
| | GGAC | | G*+A*+C | | |
| 1551 | CCTGTTCAAAGCTGC | A571549H | +C*+C*+T*G*T*T*C*A*A*A*G*C*T*G*C*T*+ | 38887 | 19 |
| | TAGG | | A*+G*+G | | |
| 1552 | CCTGTTCAAAGCTGC | A571550H | +C*+C*+T*G*T*T*C*A*A*A*G*C*T*G*C*+T* | 38888 | 18 |
| | TAG | | +A*+G | | |
| 1553 | GCTGGACTTAGCTTA | A571551Hi | +G*+C*+T*G*G*A*C*T*T*A*G*C*T*T*+A*+G | 39080 | 17 |
| | GT | | *+T | | |
| 1554 | TAGCTGGACTTAGCT | A571552Hi | +T*+A*+G*C*T*G*G*A*C*T*T*A*G*C*T*+T* | 39081 | 18 |
| | TAG | | +A*+G | | |
| 1555 | GCAAACTAGAGTGGA | A571553Hi | +G*+C*+A*A*A*C*T*A*G*A*G*T*G*G*A*+T* | 39152 | 18 |
| | TTT | | +T*+T | | |
| 1556 | ACCAAATCATTTCAG | A571554Hi | +A*+C*+C*A*A*A*T*C*A*T*T*T*C*A*G*A*+ | 39709 | 19 |
| | AGGT | | G*+G*+T | | |
| 1557 | TCTTAACATCTTTAAT | A571555Hi | +T*+C*+T*T*A*A*C*A*T*C*T*T*T*A*A*T*+ | 39907 | 19 |
| | AAA | | A*+A*+A | | |
| 1558 | CATGGAATCATTTCC | A571556Hi | +C*+A*+T*G*G*A*A*T*C*A*T*T*T*C*C*A*+ | 39983 | 19 |
| | ATTA | | T*+T*+A | | |
| 1559 | CTTTAATTAATTTATG | A571557Hi | +C*+T*+T*T*A*A*T*T*A*A*T*T*T*A*T*G*+ | 40024 | 19 |
| | ACA | | A*+C*+A | | |
| 1560 | TTATAGGCAGTGAGT | A571558Hi | +T*+T*+A*T*A*G*G*C*A*G*T*G*A*G*T*+G* | 40096 | 18 |
| | GAC | | +A*+C | | |
| 1561 | ATATTATAGGCAGTG | A571559Hi | +A*+T*+A*T*T*A*T*A*G*G*C*A*G*T*G*+A* | 40099 | 18 |
| | AGT | | +G*+T | | |
| 1562 | AAACATATTATAGGC | A571560Hi | +A*+A*+A*C*A*T*A*T*T*A*T*A*G*G*C*A*+ | 40102 | 19 |
| | AGTG | | G*+T*+G | | |
| 1563 | TTGAGTATAAATGAC | A571561Hi | +T*+T*+G*A*G*T*A*T*A*A*A*T*G*A*C*T*+ | 40119 | 19 |
| | TCAA | | C*+A*+A | | |
| 1564 | TCTTGCCATACTCAA | A571562Hi | +T*+C*+T*T*G*C*C*A*T*A*C*T*C*A*A*+G* | 40150 | 18 |
| | GGA | | +G*+A | | |
| 1565 | AAATTCTTGCCATAC | A571563Hi | +A*+A*+A*T*T*C*T*T*G*C*C*A*T*A*C*T*+ | 40153 | 19 |
| | TCAA | | C*+A*+A | | |
| 1566 | AATTCTTGCCATACT | A571564Hi | +A*+A*+T*T*C*T*T*G*C*C*A*T*A*C*T*+C* | 40153 | 18 |
| | CAA | | +A*+A | | |
| 1567 | AACATAAATTCTTGC | A571565Hi | +A*+A*+C*A*T*A*A*A*T*T*C*T*T*G*C*C*+ | 40158 | 19 |
| | CATA | | A*+T*+A | | |
| 1568 | CAACATAAATTCTTG | A571566Hi | +C*+A*+A*C*A*T*A*A*A*T*T*C*T*T*G*C*+ | 40159 | 19 |
| | CCAT | | C*+A*+T | | |
| 1569 | CGATGCTGCATCTCC | A571567Hi | +C*+G*+A*T*G*C*T*G*C*A*T*C*T*C*C*T*+ | 40356 | 19 |
| | TCTG | | C*+T*+G | | |
| 1570 | TCGATGCTGCATCTC | A571568Hi | +T*+C*+G*A*T*G*C*T*G*C*A*T*C*T*C*C*+ | 40357 | 19 |
| | CTCT | | T*+C*+T | | |
| 1571 | CTCGATGCTGCATCT | A571569Hi | +C*+T*+C*G*A*T*G*C*T*G*C*A*T*C*T*C*+ | 40358 | 19 |
| | CCTC | | C*+T*+C | | |
| 1572 | CTAGAATATGCTTAC | A571570Hi | +C*+T*+A*G*A*A*T*A*T*G*C*T*T*A*C*+T* | 40556 | 18 |
| | TAG | | +A*+G | | |
| 1573 | GTGGCAATCTTACTG | A571571Hi | +G*+T*+G*G*C*A*A*T*C*T*T*A*C*T*G*T*+ | 42238 | 19 |
| | TCAA | | C*+A*+A | | |
| 1574 | TGTGGCAATCTTACT | A571572Hi | +T*+G*+T*G*G*C*A*A*T*C*T*T*A*C*T*+G* | 42240 | 18 |
| | GTC | | +T*+C | | |
| 1575 | CTGTGGCAATCTTAC | A571573Hi | +C*+T*+G*T*G*G*C*A*A*T*C*T*T*A*C*+T* | 42241 | 18 |
| | TGT | | +G*+T | | |
| 1576 | CTGTGGCAATCTTAC | A571574Hi | +C*+T*+G*T*G*G*C*A*A*T*C*T*T*A*+C*+T | 42242 | 17 |
| | TG | | *+G | | |
| 1577 | CGGCAACTGTGGCAA | A571575Hi | +C*+G*+G*C*A*A*C*T*G*T*G*G*C*A*A*+T* | 42247 | 18 |
| | TCT | | +C*+T | | |
| 1578 | GCGGCAACTGTGGCA | A571576Hi | +G*+C*+G*G*C*A*A*C*T*G*T*G*G*C*A*A* | 42247 | 19 |
| | ATCT | | +T*+C*+T | | |
| 1579 | GCGGCAACTGTGGCA | A571577Hi | +G*+C*+G*G*C*A*A*C*T*G*T*G*G*C*A*+A | 42248 | 18 |
| | ATC | | *+T*+C | | |
| 1580 | CGCGGCAACTGTGGC | A571578Hi | +C*+G*+C*G*G*C*A*A*C*T*G*T*G*G*C*+A | 42249 | 18 |
| | AAT | | *+A*+T | | |
| 1581 | ACGCGGCAACTGTGG | A571579Hi | +A*+C*+G*C*G*G*C*A*A*C*T*G*T*G*G*C* | 42249 | 19 |
| | CAAT | | +A*+A*+T | | |
| 1582 | ACGCGGCAACTGTGG | A571580Hi | +A*+C*+G*C*G*G*C*A*A*C*T*G*T*G*G*+C | 42250 | 18 |
| | CAA | | *+A*+A | | |
| 1583 | CGCGGCAACTGTGGC | A571581Hi | +C*+G*+C*G*G*C*A*A*C*T*G*T*G*G*+C*+ | 42250 | 17 |
| | AA | | A*+A | | |
| 1584 | GTTCACAATGCTGGT | A571582Hi | +G*+T*+T*C*A*C*A*A*T*G*C*T*G*G*T*A*+ | 42369 | 19 |
| | ATCC | | T*+C*+C | | |
| 1585 | TATGTTCACAATGCT | A571583Hi | +T*+A*+T*G*T*T*C*A*C*A*A*T*G*C*T*G*+ | 42372 | 19 |
| | GGTA | | G*+T*+A | | |
| 1586 | TTTCACCAACACTCA | A571584Hi | +T*+T*+T*C*A*C*C*A*A*C*A*C*T*C*A*C*+ | 42429 | 19 |
| | CCAT | | C*+A*+T | | |
| 1587 | CTTCTCTAATAACTTT | A571585Hi | +C*+T*+T*C*T*C*T*A*A*T*A*A*C*T*T*T*+ | 42505 | 19 |
| | AGA | | A*+G*+A | | |
| 1588 | CGACTGCTGAGGTTA | A571586Hi | +C*+G*+A*C*T*G*C*T*G*A*G*G*T*T*+A*+C | 42535 | 17 |
| | CA | | *+A | | |
| 1589 | CCGACTGCTGAGGTT | A571587Hi | +C*+C*+G*A*C*T*G*C*T*G*A*G*G*T*T*+A* | 42535 | 18 |
| | ACA | | +C*+A | | |
| 1590 | CCGACTGCTGAGGTT | A571588Hi | +C*+C*+G*A*C*T*G*C*T*G*A*G*G*T*+T*+A | 42536 | 17 |
| | AC | | *+C | | |
| 1591 | TCTCCACATCACAGC | A571589Hi | +T*+C*+T*C*C*A*C*A*T*C*A*C*A*G*C*A*+ | 42570 | 19 |
| | ACTT | | C*+T*+T | | |
| 1592 | TTCTCCACATCACAG | A571590Hi | +T*+T*+C*T*C*C*A*C*A*T*C*A*C*A*G*C*+ | 42571 | 19 |
| | CACT | | A*+C*+T | | |
| 1593 | TGAAAGGCTGTGCGT | A571591Hi | +T*+G*+A*A*A*G*G*C*T*G*T*G*C*G*+T*+ | 42650 | 17 |
| | CA | | C*+A | | |
| 1594 | GTGAAAGGCTGTGCG | A571592Hi | +G*+T*+G*A*A*A*G*G*C*T*G*T*G*C*G*+T* | 42650 | 18 |
| | TCA | | +C*+A | | |
| 1595 | AGTGAAAGGCTGTGC | A571593Hi | +A*+G*+T*G*A*A*A*G*G*C*T*G*T*G*C*G* | 42650 | 19 |
| | GTCA | | +T*+C*+A | | |
| 1596 | CAGAGTAAGACATGC | A571594Hi | +C*+A*+G*A*G*T*A*A*G*A*C*A*T*G*C*A*+ | 42833 | 19 |
| | AATC | | A*+T*+C | | |
| 1597 | AGAGTAAGACATGCA | A571595Hi | +A*+G*+A*G*T*A*A*G*A*C*A*T*G*C*A*+A* | 42833 | 18 |
| | ATC | | +T*+C | | |
| 1598 | CTATTCAGAGTAAGA | A571596Hi | +C*+T*+A*T*T*C*A*G*A*G*T*A*A*G*A*C*+ | 42838 | 19 |
| | CATG | | A*+T*+G | | |
| 1599 | CTGCAATATTTGGAC | A571597Hi | +C*+T*+G*C*A*A*T*A*T*T*T*G*G*A*C*+C* | 42880 | 18 |
| | CTA | | +T*+A | | |
| 1600 | TCTGCAATATTTGGA | A571598Hi | +T*+C*+T*G*C*A*A*T*A*T*T*T*G*G*A*+C* | 42881 | 18 |
| | CCT | | +C*+T | | |
| 1601 | TGTCTGCAATATTTG | A571599Hi | +T*+G*+T*C*T*G*C*A*A*T*A*T*T*T*G*G*+ | 42882 | 19 |
| | GACC | | A*+C*+C | | |
| 1602 | GCTCTCCAAGTTTAG | A571600Hi | +G*+C*+T*C*T*C*C*A*A*G*T*T*T*A*G*A*+ | 42925 | 19 |
| | ATGT | | T*+G*+T | | |
| 1603 | CCTTATTTGGAACTC | A571601Hi | +C*+C*+T*T*A*T*T*T*G*G*A*A*C*T*C*T*+ | 42941 | 19 |
| | TGCT | | G*+C*+T | | |
| 1604 | AATCTCAAGTTCTAC | A571602Hi | +A*+A*+T*C*T*C*A*A*G*T*T*C*T*A*C*C*+ | 42955 | 19 |
| | CTTA | | T*+T*+A | | |
| 1605 | GAATCTCAAGTTCTA | A571603Hi | +G*+A*+A*T*C*T*C*A*A*G*T*T*C*T*A*C*+ | 42956 | 19 |
| | CCTT | | C*+T*+T | | |
| 1606 | TGAATCTCAAGTTCT | A571604Hi | +T*+G*+A*A*T*C*T*C*A*A*G*T*T*C*T*A*+ | 42957 | 19 |
| | ACCT | | C*+C*+T | | |
| 1607 | TGAATCTCAAGTTCT | A571605Hi | +T*+G*+A*A*T*C*T*C*A*A*G*T*T*C*T*+A* | 42958 | 18 |
| | ACC | | +C*+C | | |
| 1608 | TAAATCAGAGTTGAA | A571606Hi | +T*+A*+A*A*T*C*A*G*A*G*T*T*G*A*A*T*+ | 42968 | 19 |
| | TCTC | | C*+T*+C | | |
| 1609 | TATGTATCCAAACAT | A571607Hi | +T*+A*+T*G*T*A*T*C*C*A*A*A*C*A*T*G*+ | 43022 | 19 |
| | GGAT | | G*+A*+T | | |
| 1610 | TTATGTATCCAAACA | A571608Hi | +T*+T*+A*T*G*T*A*T*C*C*A*A*A*C*A*T*+ | 43023 | 19 |
| | TGGA | | G*+G*+A | | |
| 1611 | ACATCGCTGTTCTAG | A571609Hi | +A*+C*+A*T*C*G*C*T*G*T*T*C*T*A*G*A*+ | 43076 | 19 |
| | ACAG | | C*+A*+G | | |
| 1612 | ACATCGCTGTTCTAG | A571610Hi | +A*+C*+A*T*C*G*C*T*G*T*T*C*T*A*G*+A* | 43077 | 18 |
| | ACA | | +C*+A | | |
| 1613 | GAACATCGCTGTTCT | A571611Hi | +G*+A*+A*C*A*T*C*G*C*T*G*T*T*C*T*+A* | 43079 | 18 |
| | AGA | | +G*+A | | |
| 1614 | AATGGAACATCGCTG | A571612Hi | +A*+A*+T*G*G*A*A*C*A*T*C*G*C*T*G*+T* | 43083 | 18 |
| | TTO | | +T*+C | | |
| 1615 | AATGGAACATCGCTG | A571613Hi | +A*+A*+T*G*G*A*A*C*A*T*C*G*C*T*+G*+T | 43084 | 17 |
| | TT | | *+T | | |
| 1616 | AAATGGAACATCGCT | A571614Hi | +A*+A*+A*T*G*G*A*A*C*A*T*C*G*C*T*+G* | 43084 | 18 |
| | GTT | | +T*+T | | |
| 1617 | TAAATGGAACATCGC | A571615Hi | +T*+A*+A*A*T*G*G*A*A*C*A*T*C*G*C*+T* | 43085 | 18 |
| | TGT | | +G*+T | | |
| 1618 | GCATAAATGGAACAT | A571616Hi | +G*+C*+A*T*A*A*A*T*G*G*A*A*C*A*T*+C* | 43088 | 18 |
| | CGC | | +G*+C | | |
| 1619 | GGCATAAATGGAACA | A571617Hi | +G*+G*+C*A*T*A*A*A*T*G*G*A*A*C*A*+T* | 43089 | 18 |
| | TCG | | +C*+G | | |
| 1620 | AGGCATAAATGGAAC | A571618Hi | +A*+G*+G*C*A*T*A*A*A*T*G*G*A*A*C*A*+ | 43089 | 19 |
| | ATCG | | T*+C*+G | | |
| 1621 | AGGTGACAAAGTCTT | A571619Hi | +A*+G*+G*T*G*A*C*A*A*A*G*T*C*T*T*C*+ | 43129 | 19 |
| | CATC | | A*+T*+C | | |
| 1622 | ACAGGTGACAAAGTC | A571620Hi | +A*+C*+A*G*G*T*G*A*C*A*A*A*G*T*C*T*+ | 43131 | 19 |
| | TTCA | | T*+C*+A | | |
| 1623 | TGGAACAGGTGACAA | A571621Hi | +T*+G*+G*A*A*C*A*G*G*T*G*A*C*A*A*A*+ | 43135 | 19 |
| | AGTC | | G*+T*+C | | |
| 1624 | TCTCTCTGGAACAGG | A571622Hi | +T*+C*+T*C*T*C*T*G*G*A*A*C*A*G*G*T*+ | 43141 | 19 |
| | TGAC | | G*+A*+C | | |
| 1625 | ATCTCTCTGGAACAG | A571623Hi | +A*+T*+C*T*C*T*C*T*G*G*A*A*C*A*G*G*+ | 43142 | 19 |
| | GTGA | | T*+G*+A | | |
| 1626 | CTCTCTGGAAACAAT | A571624Hi | +C*+T*+C*T*C*T*G*G*A*A*A*C*A*A*T*A*+ | 43170 | 19 |
| | AATG | | A*+T*+G | | |
| 1627 | TACTTTACTGGATTTC | A571625Hi | +T*+A*+C*T*T*T*A*C*T*G*G*A*T*T*T*C*+ | 43265 | 19 |
| | CTC | | C*+T*+C | | |
| 1628 | TTTACTTTACTGGATT | A571626Hi | +T*+T*+T*A*C*T*T*T*A*C*T*G*G*A*T*T*+ | 43267 | 19 |
| | TCC | | T*+C*+C | | |
| 1629 | GGTTCATAAACATAG | A571627Hi | +G*+G*+T*T*C*A*T*A*A*A*C*A*T*A*G*A*+ | 43584 | 19 |
| | ATCA | | T*+C*+A | | |
| 1630 | GCTTACTTAGTTCCA | A571628Hi | +G*+C*+T*T*A*C*T*T*A*G*T*T*C*C*A*+G* | 43627 | 18 |
| | GAA | | +A*+A | | |
| 1631 | TTACACTAGCTTACTT | A571629Hi | +T*+T*+A*C*A*C*T*A*G*C*T*T*A*C*T*T*+ | 43634 | 19 |
| | AGT | | A*+G*+T | | |
| 1632 | TGGAGTTAGGCCATA | A571630Hi | +T*+G*+G*A*G*T*T*A*G*G*C*C*A*T*A*T*+ | 43714 | 19 |
| | TGAA | | G*+A*+A | | |
| 1633 | GGAGTTAGGCCATAT | A571631Hi | +G*+G*+A*G*T*T*A*G*G*C*C*A*T*A*T*+G* | 43714 | 18 |
| | GAA | | +A*+A | | |
| 1634 | TTTGGAGTTAGGCCA | A571632Hi | +T*+T*+T*G*G*A*G*T*T*A*G*G*C*C*A*+T* | 43717 | 18 |
| | TAT | | +A*+T | | |
| 1635 | AGTGCCAGTATCACA | A571633Hi | +A*+G*+T*G*C*C*A*G*T*A*T*C*A*C*A*C*+ | 43772 | 19 |
| | CCAT | | C*+A*+T | | |
| 1636 | TAGTGCCAGTATCAC | A571634Hi | +T*+A*+G*T*G*C*C*A*G*T*A*T*C*A*C*A*+ | 43773 | 19 |
| | ACCA | | C*+C*+A | | |
| 1637 | GAATATCATAACATT | A571635Hi | +G*+A*+A*T*A*T*C*A*T*A*A*C*A*T*T*A*+ | 43849 | 19 |
| | ACAA | | C*+A*+A | | |
| 1638 | GAGGAATATCATAAC | A571636Hi | +G*+A*+G*G*A*A*T*A*T*C*A*T*A*A*C*A*+ | 43852 | 19 |
| | ATTA | | T*+T*+A | | |
| 1639 | AGTCGAGGAATATCA | A571637Hi | +A*+G*+T*C*G*A*G*G*A*A*T*A*T*C*A*+T* | 43857 | 18 |
| | TAA | | +A*+A | | |
| 1640 | AGTCGAGGAATATCA | A571638Hi | +A*+G*+T*C*G*A*G*G*A*A*T*A*T*C*+A*+T | 43858 | 17 |
| | TA | | *+A | | |
| 1641 | GCAGTCGAGGAATAT | A571639Hi | +G*+C*+A*G*T*C*G*A*G*G*A*A*T*A*T*+C* | 43859 | 18 |
| | CAT | | +A*+T | | |
| 1642 | CAGTCGAGGAATATC | A571640Hi | +C*+A*+G*T*C*G*A*G*G*A*A*T*A*T*+C*+A | 43859 | 17 |
| | AT | | *+T | | |
| 1643 | GTTCCAACACAAGTA | A571641Hi | +G*+T*+T*C*C*A*A*C*A*C*A*A*G*T*A*A*+ | 43896 | 19 |
| | ACTG | | C*+T*+G | | |
| 1644 | TTGTTCCAACACAAG | A571642Hi | +T*+T*+G*T*T*C*C*A*A*C*A*C*A*A*G*T*+ | 43898 | 19 |
| | TAAC | | A*+A*+C | | |
| 1645 | TTAGTTGTTCCAACA | A571643Hi | +T*+T*+A*G*T*T*G*T*T*C*C*A*A*C*A*C*+ | 43902 | 19 |
| | CAAG | | A*+A*+G | | |
| 1646 | AGTTAGTTGTTCCAA | A571644Hi | +A*+G*+T*T*A*G*T*T*G*T*T*C*C*A*A*C*+ | 43904 | 19 |
| | CACA | | A*+C*+A | | |
| 1647 | GAGTTAGTTGTTCCA | A571645Hi | +G*+A*+G*T*T*A*G*T*T*G*T*T*C*C*A*A*+ | 43905 | 19 |
| | ACAC | | C*+A*+C | | |
| 1648 | GAGTTAGTTGTTCCA | A571646Hi | +G*+A*+G*T*T*A*G*T*T*G*T*T*C*C*A*+A* | 43906 | 18 |
| | ACA | | +C*+A | | |
| 1649 | TGAAGAGTTAGTTGT | A571647Hi | +T*+G*+A*A*G*A*G*T*T*A*G*T*T*G*T*T*+ | 43909 | 19 |
| | TCCA | | C*+C*+A | | |
| 1650 | TGAAGAGTTAGTTGT | A571648Hi | +T*+G*+A*A*G*A*G*T*T*A*G*T*T*G*T*+T* | 43910 | 18 |
| | TCC | | +C*+C | | |
| 1651 | GCTGAAGAGTTAGTT | A571649Hi | +G*+C*+T*G*A*A*G*A*G*T*T*A*G*T*T*G*+ | 43911 | 19 |
| | GTTC | | T*+T*+C | | |
| 1652 | TCTCATCCATCTGTA | A571650Hi | +T*+C*+T*C*A*T*C*C*A*T*C*T*G*T*A*A*+ | 43984 | 19 |
| | ATTT | | T*+T*+T | | |
| 1653 | AATCTCATCCATCTG | A571651Hi | +A*+A*+T*C*T*C*A*T*C*C*A*T*C*T*G*T*+ | 43986 | 19 |
| | TAAT | | A*+A*+T | | |
| 1654 | TTACGTTAATAACAA | A571652Hi | +T*+T*+A*C*G*T*T*A*A*T*A*A*C*A*A*T*+ | 44053 | 19 |
| | TTAT | | T*+A*+T | | |
| 1655 | TTTACGTTAATAACA | A571653Hi | +T*+T*+T*A*C*G*T*T*A*A*T*A*A*C*A*A*+ | 44054 | 19 |
| | ATTA | | T*+*+A | | |
| 1656 | TTTACGTTAATAACA | A571654Hi | +T*+T*+T*A*C*G*T*T*A*A*T*A*A*C*A*+A* | 44055 | 18 |
| | ATT | | +T*+T | | |
| 1657 | TCATTTACGTTAATAA | A571655Hi | +T*+C*+A*T*T*T*A*C*G*T*T*A*A*T*A*A*+ | 44057 | 19 |
| | CAA | | C*+A*+A | | |
| 1658 | ATGAAATTGCTGATC | A571656Hi | +A*+T*+G*A*A*A*T*T*G*C*T*G*A*T*C*+A* | 44138 | 18 |
| | AGA | | +G*+A | | |
| 1659 | AATGAAATTGCTGAT | A571657Hi | +A*+A*+T*G*A*A*A*T*T*G*C*T*G*A*T*+C* | 44139 | 18 |
| | CAG | | +A*+G | | |
| 1660 | GCATGAATGATTACC | A571658Hi | +G*+C*+A*T*G*A*A*T*G*A*T*T*A*C*C*A*+ | 44268 | 19 |
| | ATGT | | T*+G*+T | | |
| 1661 | CAGCATGAATGATTA | A571659Hi | +C*+A*+G*C*A*T*G*A*A*T*G*A*T*T*A*C*+ | 44270 | 19 |
| | CCAT | | C*+A*+T | | |
| 1662 | GCTTCAACCTAACTA | A571660Hi | +G*+C*+T*T*C*A*A*C*C*T*A*A*C*T*A*+C* | 44340 | 18 |
| | CTA | | +T*+A | | |
| 1663 | ACTCTGCTTGTTGTA | A571661Hi | +A*+C*+T*C*T*G*C*T*T*G*T*T*G*T*A*G*+ | 44492 | 19 |
| | GTCC | | T*+C*+C | | |
| 1664 | GACTCTGCTTGTTGT | A571662Hi | +G*+A*+C*T*C*T*G*C*T*T*G*T*T*G*T*A*+ | 44493 | 19 |
| | AGTC | | G*+T*+C | | |
| 1665 | GGACTCTGCTTGTTG | A571663Hi | +G*+G*+A*C*T*C*T*G*C*T*T*G*T*T*G*T*+ | 44494 | 19 |
| | TAGT | | A*+G*+T | | |
| 1666 | GACTCTGCTTGTTGT | A571664Hi | +G*+A*+C*T*C*T*G*C*T*T*G*T*T*G*T*+A* | 44494 | 18 |
| | AGT | | +G*+T | | |
| 1667 | GGACTCTGCTTGTTG | A571665Hi | +G*+G*+A*C*T*C*T*G*C*T*T*G*T*T*G*+T* | 44495 | 18 |
| | TAG | | +A*+G | | |
| 1668 | GACTCTGCTTGTTGT | A571666Hi | +G*+A*+C*T*C*T*G*C*T*T*G*T*T*G*+T*+A | 44495 | 17 |
| | AG | | *+G | | |
| 1669 | TGTGTTGCAGGTCAG | A571667Hi | +T*+G*+T*G*T*T*G*C*A*G*G*T*C*A*G*G*+ | 44513 | 19 |
| | GAGG | | A*+G*+G | | |
| 1670 | TATGTGTTGCAGGTC | A571668Hi | +T*+A*+T*G*T*G*T*T*G*C*A*G*G*T*C*+A* | 44516 | 18 |
| | AGG | | +G*+G | | |
| 1671 | GCTACATATGTGTTG | A571669Hi | +G*+C*+T*A*C*A*T*A*T*G*T*G*T*T*G*+C* | 44522 | 18 |
| | CAG | | +A*+G | | |
| 1672 | TGCTACATATGTGTT | A571670Hi | +T*+G*+C*T*A*C*A*T*A*T*G*T*G*T*T*+G* | 44523 | 18 |
| | GCA | | +C*+A | | |
| 1673 | ATGCTACATATGTGT | A571671Hi | +A*+T*+G*C*T*A*C*A*T*A*T*G*T*G*T*T*+ | 44523 | 19 |
| | TGCA | | G*+C*+A | | |
| 1674 | CATGCTACATATGTG | A571672Hi | +C*+A*+T*G*C*T*A*C*A*T*A*T*G*T*G*T*+ | 44524 | 19 |
| | TTGC | | T*+G*+C | | |
| 1675 | TCATGCTACATATGT | A571673Hi | +T*+C*+A*T*G*C*T*A*C*A*T*A*T*G*T*G*+ | 44525 | 19 |
| | GTTG | | T*+T*+G | | |
| 1676 | CATGCTACATATGTG | A571674Hi | +C*+A*+T*G*C*T*A*C*A*T*A*T*G*T*G*+T* | 44525 | 18 |
| | TTG | | +T*+G | | |
| 1677 | CCATTATTGCTCATG | A571675Hi | +C*+C*+A*T*T*A*T*T*G*C*T*C*A*T*G*C*+ | 44535 | 19 |
| | CTAC | | T*+A*+C | | |
| 1678 | TTAAATTAAGCTGCA | A571676Hi | +T*+T*+A*A*A*T*T*A*A*G*C*T*G*C*A*G*+ | 44593 | 19 |
| | GTAA | | T*+A*+A | | |
| 1679 | TAATTTATCTGATTAG | A571677Hi | +T*+A*+A*T*T*T*A*T*C*T*G*A*T*T*A*G*+ | 44643 | 19 |
| | TCT | | T*+C*+T | | |
| 1680 | AATGGTATAATTTAT | A571678Hi | +A*+A*+T*G*G*T*A*T*A*A*T*T*T*A*T*C*+ | 44650 | 19 |
| | CTGA | | T*+G*+A | | |
| 1681 | TTGGAAATGGTATAA | A571679Hi | +T*+T*+G*G*A*A*A*T*G*G*T*A*T*A*A*T*+ | 44655 | 19 |
| | TTTA | | T*+T*+A | | |
| 1682 | ATTTGGAAATGGTAT | A571680Hi | +A*+T*+T*T*G*G*A*A*A*T*G*G*T*A*T*A*+ | 44657 | 19 |
| | AATT | | A*+T*+T | | |
| 1683 | TAGGAATCTCACCTC | A571681Hi | +T*+A*+G*G*A*A*T*C*T*C*A*C*C*T*C*A*+ | 44704 | 19 |
| | ATTG | | T*+T*+G | | |
| 1684 | ACATTAGGAATCTCA | A571682Hi | +A*+C*+A*T*T*A*G*G*A*A*T*C*T*C*A*C*+ | 44708 | 19 |
| | CCTC | | C*+T*+C | | |
| 1685 | GCAGCACATTAGGAA | A571683Hi | +G*+C*+A*G*C*A*C*A*T*T*A*G*G*A*A*T*+ | 44713 | 19 |
| | TCTC | | C*+T*+C | | |
| 1686 | AGCAGCACATTAGGA | A571684Hi | +A*+G*+C*A*G*C*A*C*A*T*T*A*G*G*A*A*+ | 44714 | 19 |
| | ATCT | | T*+C*+T | | |
| 1687 | CAAACTTAGCTATTA | A571685Hi | +C*+A*+A*A*C*T*T*A*G*C*T*A*T*T*A*G*+ | 44930 | 19 |
| | GAAA | | A*+A*+A | | |
| 1688 | CCACAAACTTAGCTA | A571686Hi | +C*+C*+A*C*A*A*A*C*T*T*A*G*C*T*A*+T* | 44934 | 18 |
| | TTA | | +T*+A | | |
| 1689 | CATTAAGTACTTGTC | A571687Hi | +C*+A*+T*T*A*A*G*T*A*C*T*T*G*T*C*C*+ | 45010 | 19 |
| | CAGG | | A*+G*+G | | |
| 1690 | ACATTAAGTACTTGT | A571688Hi | +A*+C*+A*T*T*A*A*G*T*A*C*T*T*G*T*C*+ | 45011 | 19 |
| | CCAG | | C*+A*+G | | |
| 1691 | CATTAAGTACTTGTC | A571689Hi | +C*+A*+T*T*A*A*G*T*A*C*T*T*G*T*C*+C* | 45011 | 18 |
| | CAG | | +A*+G | | |
| 1692 | GATGAAGACTGCTTC | A571690Hi | +G*+A*+T*G*A*A*G*A*C*T*G*C*T*T*C*C*+ | 45368 | 19 |
| | CAAA | | A*+A*+A | | |
| 1693 | GAGATGAAGACTGCT | A571691Hi | +G*+A*+G*A*T*G*A*A*G*A*C*T*G*C*T*T*+ | 45370 | 19 |
| | TCCA | | C*+C*+A | | |
| 1694 | TGAGATGAAGACTGC | A571692Hi | +T*+G*+A*G*A*T*G*A*A*G*A*C*T*G*C*T*+ | 45371 | 19 |
| | TTCC | | T*+C*+C | | |
| 1695 | AGGACTTGAGATGAA | A571693Hi | +A*+G*+G*A*C*T*T*G*A*G*A*T*G*A*A*G*+ | 45377 | 19 |
| | GACT | | A*+C*+T | | |
| 1696 | AAGGACTTGAGATGA | A571694Hi | +A*+A*+G*G*A*C*T*T*G*A*G*A*T*G*A*A*+ | 45378 | 19 |
| | AGAC | | G*+A*+C | | |
| 1697 | TCAGTACTAGAACAC | A571695Hi | +T*+C*+A*G*T*A*C*T*A*G*A*A*C*A*C*A*+ | 45576 | 19 |
| | AGTG | | G*+T*+G | | |
| 1698 | TTCAGTACTAGAACA | A571696Hi | +T*+T*+C*A*G*T*A*C*T*A*G*A*A*C*A*C*+ | 45577 | 19 |
| | CAGT | | A*+G*+T | | |
| 1699 | ATGTATAGCTAGAAT | A571697Hi | +A*+T*+G*T*A*T*A*G*C*T*A*G*A*A*T*+G* | 45732 | 18 |
| | GAG | | +A*+G | | |
| 1700 | TTAATATCTTTACCAT | A571698Hi | +T*+T*+A*A*T*A*T*C*T*T*T*A*C*C*A*T*+ | 45817 | 19 |
| | GTA | | G*+T*+A | | |
| 1701 | GTATATCCAGTTCTA | A571699Hi | +G*+T*+A*T*A*T*C*C*A*G*T*T*C*T*A*C*+ | 45916 | 19 |
| | CTTC | | T*+T*+C | | |
| 1702 | AGTAAGTATATCCAG | A571700Hi | +A*+G*+T*A*A*G*T*A*T*A*T*C*C*A*G*T*+ | 45921 | 19 |
| | TTCT | | T*+C*+T | | |
| 1703 | ATCATATGACTGATA | A571701Hi | +A*+T*+C*A*T*A*T*G*A*C*T*G*A*T*A*+G* | 46128 | 18 |
| | GCC | | +C*+C | | |
| 1704 | ACATCATATGACTGA | A571702Hi | +A*+C*+A*T*C*A*T*A*T*G*A*C*T*G*A*T*+ | 46129 | 19 |
| | TAGC | | A*+G*+C | | |
| 1705 | AACATCATATGACTG | A571703Hi | +A*+A*+C*A*T*C*A*T*A*T*G*A*C*T*G*A*+ | 46130 | 19 |
| | ATAG | | T*+A*+G | | |
| 1706 | AGAGATCCAACATCA | A571704Hi | +A*+G*+A*G*A*T*C*C*A*A*C*A*T*C*A*T*+ | 46138 | 19 |
| | TATG | | A*+T*+G | | |
| 1707 | TAGGTAAGTAGATTA | A571705Hi | +T*+A*+G*G*T*A*A*G*T*A*G*A*T*T*A*+G* | 46358 | 18 |
| | GCC | | +C*+C | | |
| 1708 | CTACTAGGTAAGTAG | A571706Hi | +C*+T*+A*C*T*A*G*G*T*A*A*G*T*A*G*+A* | 46362 | 18 |
| | ATT | | +T*+T | | |
| 1709 | GCTACTAGGTAAGTA | A571707Hi | +G*+C*+T*A*C*T*A*G*G*T*A*A*G*T*A*G*+ | 46362 | 19 |
| | GATT | | A*+T*+T | | |
| 1710 | AGCTACTAGGTAAGT | A571708Hi | +A*+G*+C*T*A*C*T*A*G*G*T*A*A*G*T*A*+ | 46363 | 19 |
| | AGAT | | G*+A*+T | | |
| 1711 | AGCTACTAGGTAAGT | A571709Hi | +A*+G*+C*T*A*C*T*A*G*G*T*A*A*G*T*+A* | 46364 | 18 |
| | AGA | | +G*+A | | |
| 1712 | CTAGCTACTAGGTAA | A571710Hi | +C*+T*+A*G*C*T*A*C*T*A*G*G*T*A*A*G*+ | 46365 | 19 |
| | GTAG | | T*+A*+G | | |
| 1713 | CTAGCTACTAGGTAA | A571711Hi | +C*+T*+A*G*C*T*A*C*T*A*G*G*T*A*A*+G* | 46366 | 18 |
| | GTA | | +T*+A | | |
| 1714 | TGGCAGCATCCTACC | A571712Hi | +T*+G*+G*C*A*G*C*A*T*C*C*T*A*C*C*+A* | 46496 | 18 |
| | ACC | | +C*+C | | |
| 1715 | GAAGGATGGCAGCAT | A571713Hi | +G*+A*+A*G*G*A*T*G*G*C*A*G*C*A*T*C* | 46501 | 19 |
| | CCTA | | +C*+T*+A | | |
| 1716 | AGAAGGATGGCAGCA | A571714Hi | +A*+G*+A*A*G*G*A*T*G*G*C*A*G*C*A*T* | 46502 | 19 |
| | TCCT | | +C*+C*+T | | |
| 1717 | CAACAGAAGGATGGC | A571715Hi | +C*+A*+A*C*A*G*A*A*G*G*A*T*G*G*C*A* | 46506 | 19 |
| | AGCA | | +G*+C*+A | | |
| 1718 | CAACAGAAGGATGGC | A571716Hi | +C*+A*+A*C*A*G*A*A*G*G*A*T*G*G*C*+A | 46507 | 18 |
| | AGC | | *+G*+C | | |
| 1719 | CCAACAGAAGGATGG | A571717Hi | +C*+C*+A*A*C*A*G*A*A*G*G*A*T*G*G*C* | 46507 | 19 |
| | CAGC | | +A*+G*+C | | |
| 1720 | GGAGATGCCAACAGA | A571718Hi | +G*+G*+A*G*A*T*G*C*C*A*A*C*A*G*A*A* | 46514 | 19 |
| | AGGA | | +G*+G*+A | | |
| 1721 | TGGAGATGCCAACAG | A571719Hi | +T*+G*+G*A*G*A*T*G*C*C*A*A*C*A*G*A*+ | 46515 | 19 |
| | AAGG | | A*+G*+G | | |
| 1722 | GGAGATGCCAACAGA | A571720Hi | +G*+G*+A*G*A*T*G*C*C*A*A*C*A*G*A*+A | 46515 | 18 |
| | AGG | | *+G*+G | | |
| 1723 | CTCTGGAGATGCCAA | A571721Hi | +C*+T*+C*T*G*G*A*G*A*T*G*C*C*A*A*C*+ | 46518 | 19 |
| | CAGA | | A*+G*+A | | |
| 1724 | ACTCTGGAGATGCCA | A571722Hi | +A*+C*+T*C*T*G*G*A*G*A*T*G*C*C*A*A*+ | 46519 | 19 |
| | ACAG | | C*+A*+G | | |
| 1725 | AGTGAAACTCTGGAG | A571723Hi | +A*+G*+T*G*A*A*A*C*T*C*T*G*G*A*G*A*+ | 46525 | 19 |
| | ATGC | | T*+G*+C | | |
| 1726 | TCAAGTGAAACTCTG | A571724Hi | +T*+C*+A*A*G*T*G*A*A*A*C*T*C*T*G*G*+ | 46528 | 19 |
| | GAGA | | A*+G*+A | | |
| 1727 | GTCTACTTAGGAGGT | A571725Hi | +G*+T*+C*T*A*C*T*T*A*G*G*A*G*G*T*+G* | 46549 | 18 |
| | GTT | | +T*+T | | |
| 1728 | AGTGTCTACTTAGGA | A571726Hi | +A*+G*+T*G*T*C*T*A*C*T*T*A*G*G*A*+G* | 46552 | 18 |
| | GGT | | +G*+T | | |
| 1729 | CTAGGCTCTAACCAT | A571727Hi | +C*+T*+A*G*G*C*T*C*T*A*A*C*C*A*T*+T* | 46888 | 18 |
| | TTC | | +T*+C | | |
| 1730 | ATTATATCAAATTGAT | A571728Hi | +A*+T*+T*A*T*A*T*C*A*A*A*T*T*G*A*T*+ | 46988 | 19 |
| | ATT | | A*+T*+T | | |
| 1731 | AATTATATCAAATTG | A571729Hi | +A*+A*+T*T*A*T*A*T*C*A*A*A*T*T*G*A*+ | 46989 | 19 |
| | ATAT | | T*+A*+T | | |
| 1732 | GTCATTCTGAACTTT | A571730Hi | +G*+T*+C*A*T*T*C*T*G*A*A*C*T*T*T*C*+ | 47247 | 19 |
| | CTGT | | T*+G*+T | | |
| 1733 | AGTCAGATTGTCATT | A571731Hi | +A*+G*+T*C*A*G*A*T*T*G*T*C*A*T*T*C*+ | 47256 | 19 |
| | CTGA | | T*+G*+A | | |
| 1734 | GTCAAAGCACAAGTT | A571732Hi | +G*+T*+C*A*A*A*G*C*A*C*A*A*G*T*T*T*+ | 47371 | 19 |
| | TAAA | | A*+A*+A | | |
| 1735 | ACTTCATATGTATTCA | A571733Hi | +A*+C*+T*T*C*A*T*A*T*G*T*A*T*T*C*A*+ | 47487 | 19 |
| | CCT | | C*+C*+T | | |
| 1736 | ACTTCATATGTATTCA | A571734Hi | +A*+C*+T*T*C*A*T*A*T*G*T*A*T*T*C*+A* | 47488 | 18 |
| | CC | | +C*+C | | |
| 1737 | AACAATAGCAATGAC | A571735Hi | +A*+A*+C*A*A*T*A*G*C*A*A*T*G*A*C*C*+ | 47561 | 19 |
| | CACA | | A*+C*+A | | |
| 1738 | TAACAACTTAATTGG | A571736Hi | +T*+A*+A*C*A*A*C*T*T*A*A*T*T*G*G*T*+ | 47757 | 19 |
| | TGCT | | G*+C*+T | | |
| 1739 | GCGCTGTCCTCAGAG | A571737Hi | +G*+C*+G*C*T*G*T*C*C*T*C*A*G*A*G*A*+ | 47973 | 19 |
| | ATGA | | T*+G*+A | | |
| 1740 | TAAGCGCTGTCCTCA | A571738Hi | +T*+A*+A*G*C*G*C*T*G*T*C*C*T*C*A*G*+ | 47976 | 19 |
| | GAGA | | A*+G*+A | | |
| 1741 | AAATTGGAAGGCTGG | A571739Hi | +A*+A*+A*T*T*G*G*A*A*G*G*C*T*G*G*T*+ | 48124 | 19 |
| | TTTG | | T*+T*+G | | |
| 1742 | TGAATGAAGTAACAA | A571740Hi | +T*+G*+A*A*T*G*A*A*G*T*A*A*C*A*A*T*+ | 48233 | 19 |
| | TTAC | | T*+A*+C | | |
| 1743 | TCTGAGAACCATTAA | A571741Hi | +T*+C*+T*G*A*G*A*A*C*C*A*T*T*A*A*A*+ | 48249 | 19 |
| | ATGA | | T*+G*+A | | |
| 1744 | GAATCTGAGAACCAT | A571742Hi | +G*+A*+A*T*C*T*G*A*G*A*A*C*C*A*T*T*+ | 48252 | 19 |
| | TAAA | | A*+A*+A | | |
| 1745 | CAGCTTGGAGGCCAA | A571743Hi | +C*+A*+G*C*T*T*G*G*A*G*G*C*C*A*A*+T* | 48291 | 18 |
| | TTA | | +T*+A | | |
| 1746 | TCAGCTTGGAGGCCA | A571744Hi | +T*+C*+A*G*C*T*T*G*G*A*G*G*C*C*A*+A* | 48292 | 18 |
| | ATT | | +T*+T | | |
| 1747 | ATCAGCTTGGAGGCC | A571745Hi | +A*+T*+C*A*G*C*T*T*G*G*A*G*G*C*C*A*+ | 48292 | 19 |
| | AATT | | A*+T*+T | | |
| 1748 | ATCAGCTTGGAGGCC | A571746Hi | +A*+T*+C*A*G*C*T*T*G*G*A*G*G*C*C*+A* | 48293 | 18 |
| | AAT | | +A*+T | | |
| 1749 | ACATCAGCTTGGAGG | A571747Hi | +A*+C*+A*T*C*A*G*C*T*T*G*G*A*G*G*C*+ | 48294 | 19 |
| | CCAA | | C*+A*+A | | |
| 1750 | GCACATCAGCTTGGA | A571748Hi | +G*+C*+A*C*A*T*C*A*G*C*T*T*G*G*A*G*+ | 48296 | 19 |
| | GGCC | | G*+C*+C | | |
| 1751 | GCACATCAGCTTGGA | A571749Hi | +G*+C*+A*C*A*T*C*A*G*C*T*T*G*G*A*+G* | 48297 | 18 |
| | GGC | | +G*+C | | |
| 1752 | AGCACATCAGCTTGG | A571750Hi | +A*+G*+C*A*C*A*T*C*A*G*C*T*T*G*G*A*+ | 48297 | 19 |
| | AGGC | | G*+G*+C | | |
| 1753 | TAAGCACATCAGCTT | A571751Hi | +T*+A*+A*G*C*A*C*A*T*C*A*G*C*T*T*G*+ | 48299 | 19 |
| | GGAG | | G*+A*+G | | |
| 1754 | TGTTTCCTGTCACTG | A571752Hi | +T*+G*+T*T*T*C*C*T*G*T*C*A*C*T*G*T*+ | 48320 | 19 |
| | TTTC | | T*+T*+C | | |
| 1755 | TTGCTCAGCTTGGAC | A571753Hi | +T*+T*+G*C*T*C*A*G*C*T*T*G*G*A*C*A*+ | 48383 | 19 |
| | ATCA | | T*+C*+A | | |
| 1756 | TTGCTCAGCTTGGAC | A571754Hi | +T*+T*+G*C*T*C*A*G*C*T*T*G*G*A*C*+A* | 48384 | 18 |
| | ATC | | +T*+C | | |
| 1757 | ATTGCTCAGCTTGGA | A571755Hi | +A*+T*+T*G*C*T*C*A*G*C*T*T*G*G*A*C*+ | 48384 | 19 |
| | CATC | | A*+T*+C | | |
| 1758 | TAATTGCTCAGCTTG | A571756Hi | +T*+A*+A*T*T*G*C*T*C*A*G*C*T*T*G*G*+ | 48386 | 19 |
| | GACA | | A*+C*+A | | |
| 1759 | TTAATTGCTCAGCTT | A571757Hi | +T*+T*+A*A*T*T*G*C*T*C*A*G*C*T*T*G*+ | 48387 | 19 |
| | GGAC | | G*+A*+C | | |
| 1760 | TTTAATTGCTCAGCTT | A571758Hi | +T*+T*+T*A*A*T*T*G*C*T*C*A*G*C*T*T*+ | 48388 | 19 |
| | GGA | | G*+G*+A | | |
| 1761 | ACTTCACACTTTAATT | A571759Hi | +A*+C*+T*T*C*A*C*A*C*T*T*T*A*A*T*T*+ | 48397 | 19 |
| | GCT | | G*+C*+T | | |
| 1762 | TAATGCCTCTCAATT | A571760Hi | +T*+A*+A*T*G*C*C*T*C*T*C*A*A*T*T*A*+ | 48479 | 19 |
| | ATCT | | T*+C*+T | | |
| 1763 | CTGTACTCTCAGTGA | A571761Hi | +C*+T*+G*T*A*C*T*C*T*C*A*G*T*G*A*A*+ | 48527 | 19 |
| | ACAG | | C*+A*+G | | |
| 1764 | TGTACTCTCAGTGAA | A571762Hi | +T*+G*+T*A*C*T*C*T*C*A*G*T*G*A*A*+C* | 48527 | 18 |
| | CAG | | +A*+G | | |
| 1765 | TTCTGTACTCTCAGT | A571763Hi | +T*+T*+C*T*G*T*A*C*T*C*T*C*A*G*T*G*+ | 48529 | 19 |
| | GAAC | | A*+A*+C | | |
| 1766 | TCCTCATTTCTGTACT | A571764Hi | +T*+C*+C*T*C*A*T*T*T*C*T*G*T*A*C*T*+ | 48536 | 19 |
| | CTC | | C*+T*+C | | |
| 1767 | AATCTTCTGCCTTCC | A571765Hi | +A*+A*+T*C*T*T*C*T*G*C*C*T*T*C*C*A*+ | 48556 | 19 |
| | AAGC | | A*+G*+C | | |
| 1768 | CAGATAATCCAAAGA | A571766Hi | +C*+A*+G*A*T*A*A*T*C*C*A*A*A*G*A*G*+ | 48606 | 19 |
| | GCTG | | C*+T*+G | | |
| 1769 | TGGCCTCCACCACAG | A571767Hi | +T*+G*+G*C*C*T*C*C*A*C*C*A*C*A*G*+A* | 48619 | 18 |
| | ATA | | +T*+A | | |
| 1770 | GATGGCCTCCACCAC | A571768Hi | +G*+A*+T*G*G*C*C*T*C*C*A*C*C*A*C*A*+ | 48620 | 19 |
| | AGAT | | G*+A*+T | | |
| 1771 | TGATGGCCTCCACCA | A571769Hi | +T*+G*+A*T*G*G*C*C*T*C*C*A*C*C*A*C*+ | 48621 | 19 |
| | CAGA | | A*+G*+A | | |
| 1772 | GATGGCCTCCACCAC | A571770Hi | +G*+A*+T*G*G*C*C*T*C*C*A*C*C*A*C*+A* | 48621 | 18 |
| | AGA | | +G*+A | | |
| 1773 | GTGATGGCCTCCACC | A571771Hi | +G*+T*+G*A*T*G*G*C*C*T*C*C*A*C*C*A*+ | 48622 | 19 |
| | ACAG | | C*+A*+G | | |
| 1774 | CGTGATGGCCTCCAC | A571772Hi | +C*+G*+T*G*A*T*G*G*C*C*T*C*C*A*C*C*+ | 48623 | 19 |
| | CACA | | A*+C*+A | | |
| 1775 | CGTGATGGCCTCCAC | A571773Hi | +C*+G*+T*G*A*T*G*G*C*C*T*C*C*A*C*+C* | 48624 | 18 |
| | CAC | | +A*+C | | |
| 1776 | ACGTGATGGCCTCCA | A571774Hi | +A*+C*+G*T*G*A*T*G*G*C*C*T*C*C*A*C*+ | 48624 | 19 |
| | CCAC | | C*+A*+C | | |
| 1777 | GACGTGATGGCCTCC | A571775Hi | +G*+A*+C*G*T*G*A*T*G*G*C*C*T*C*C*A*+ | 48625 | 19 |
| | ACCA | | C*+C*+A | | |
| 1778 | AAGACGTGATGGCCT | A571776Hi | +A*+A*+G*A*C*G*T*G*A*T*G*G*C*C*T*+C* | 48628 | 18 |
| | CCA | | +C*+A | | |
| 1779 | AAGACGTGATGGCCT | A571777Hi | +A*+A*+G*A*C*G*T*G*A*T*G*G*C*C*+T*+ | 48629 | 17 |
| | CC | | C*+C | | |
| 1780 | CAAAGACGTGATGGC | A571778Hi | +C*+A*+A*A*G*A*C*G*T*G*A*T*G*G*C*+C* | 48630 | 18 |
| | CTC | | +T*+C | | |
| 1781 | CCAAAGACGTGATGG | A571779Hi | +C*+C*+A*A*A*G*A*C*G*T*G*A*T*G*G*C*+ | 48630 | 19 |
| | CCTC | | C*+T*+C | | |
| 1782 | CCACTCCAAAGACGT | A571780Hi | +C*+C*+A*C*T*C*C*A*A*A*G*A*C*G*T*G*+ | 48635 | 19 |
| | GATG | | A*+T*+G | | |
| 1783 | CCACTCCAAAGACGT | A571781Hi | +C*+C*+A*C*T*C*C*A*A*A*G*A*C*G*T*+G* | 48636 | 18 |
| | GAT | | +A*+T | | |
| 1784 | TCCACTCCAAAGACG | A571782Hi | +T*+C*+C*A*C*T*C*C*A*A*A*G*A*C*G*T*+ | 48636 | 19 |
| | TGAT | | G*+A*+T | | |
| 1785 | CTCCACTCCAAAGAC | A571783Hi | +C*+T*+C*C*A*C*T*C*C*A*A*A*G*A*C*G*+ | 48637 | 19 |
| | GTGA | | T*+G*+A | | |
| 1786 | CCTCCACTCCAAAGA | A571784Hi | +C*+C*+T*C*C*A*C*T*C*C*A*A*A*G*A*C*+ | 48638 | 19 |
| | CGTG | | G*+T*+G | | |
| 1787 | ACCTCCACTCCAAAG | A571785Hi | +A*+C*+C*T*C*C*A*C*T*C*C*A*A*A*G*A*+ | 48639 | 19 |
| | ACGT | | C*+G*+T | | |
| 1788 | TACCTCCACTCCAAA | A571786Hi | +T*+A*+C*C*T*C*C*A*C*T*C*C*A*A*A*G*+ | 48640 | 19 |
| | GACG | | A*+C*+G | | |
| 1789 | ACCTCCACTCCAAAG | A571787Hi | +A*+C*+C*T*C*C*A*C*T*C*C*A*A*A*G*+A* | 48640 | 18 |
| | ACG | | +C*+G | | |
| 1790 | GTACCTCCACTCCAA | A571788Hi | +G*+T*+A*C*C*T*C*C*A*C*T*C*C*A*A*A*+ | 48641 | 19 |
| | AGAC | | G*+A*+C | | |
| 1791 | GGTACCTCCACTCCA | A571789Hi | +G*+G*+T*A*C*C*T*C*C*A*C*T*C*C*A*A*+ | 48642 | 19 |
| | AAGA | | A*+G*+A | | |
| 1792 | CATGGTACCTCCACT | A571790Hi | +C*+A*+T*G*G*T*A*C*C*T*C*C*A*C*T*+C* | 48646 | 18 |
| | CCA | | +C*+A | | |
| 1793 | CTCTTATTCTGGTCAT | A571791Hi | +C*+T*+C*T*T*A*T*T*C*T*G*G*T*C*A*T*+ | 48807 | 19 |
| | CTC | | C*+T*+C | | |
| 1794 | CCTACTCATCAACCA | A571792Hi | +C*+C*+T*A*C*T*C*A*T*C*A*A*C*C*A*+A* | 48848 | 18 |
| | AGA | | +G*+A | | |
| 1795 | TAATAGGTTTGCCAA | A571793Hi | +T*+A*+A*T*A*G*G*T*T*T*G*C*C*A*A*A*+ | 49060 | 19 |
| | ACTT | | C*+T*+T | | |
| 1796 | CATGTAATAGGTTTG | A571794Hi | +C*+A*+T*G*T*A*A*T*A*G*G*T*T*T*G*C*+ | 49064 | 19 |
| | CCAA | | C*+A*+A | | |
| 1797 | CATGTAATAGGTTTG | A571795Hi | +C*+A*+T*G*T*A*A*T*A*G*G*T*T*T*G*+C* | 49065 | 18 |
| | CCA | | +C*+A | | |
| 1798 | CATGTAATAGGTTTG | A571796Hi | +C*+A*+T*G*T*A*A*T*A*G*G*T*T*T*+G*+C | 49066 | 17 |
| | CC | | *+C | | |
| 1799 | CTTCATGTAATAGGT | A571797Hi | +C*+T*+T*C*A*T*G*T*A*A*T*A*G*G*T*+T* | 49068 | 18 |
| | TTG | | +T*+G | | |
| 1800 | AGCTTCATGTAATAG | A571798Hi | +A*+G*+C*T*T*C*A*T*G*T*A*A*T*A*G*G*+ | 49069 | 19 |
| | GTTT | | T*+T*+T | | |
| 1801 | CATGAAATCCATACT | A571799Hi | +C*+A*+T*G*A*A*A*T*C*C*A*T*A*C*T*G*+ | 49316 | 19 |
| | GATA | | A*+T*+A | | |
| 1802 | TGAAGAATAAGAGTT | A571800Hi | +T*+G*+A*A*G*A*A*T*A*A*G*A*G*T*T*A*+ | 49412 | 19 |
| | AGCA | | G*+C*+A | | |
| 1803 | TTAAGACCAGTTATA | A571801Hi | +T*+T*+A*A*G*A*C*C*A*G*T*T*A*T*A*+G* | 49515 | 18 |
| | GAC | | +A*+C | | |
| 1804 | TAGTTAAGACCAGTT | A571802Hi | +T*+A*+G*T*T*A*A*G*A*C*C*A*G*T*T*+A* | 49518 | 18 |
| | ATA | | +T*+A | | |
| 1805 | TTAGTTAAGACCAGT | A571803Hi | +T*+T*+A*G*T*T*A*A*G*A*C*C*A*G*T*+T* | 49519 | 18 |
| | TAT | | +A*+T | | |
| 1806 | CCTTAGTTAAGACCA | A571804Hi | +C*+C*+T*T*A*G*T*T*A*A*G*A*C*C*A*G*+ | 49520 | 19 |
| | GTTA | | T*+T*+A | | |
| 1807 | CTTACCTTAGTTAAG | A571805Hi | +C*+T*+T*A*C*C*T*T*A*G*T*T*A*A*G*A*+ | 49524 | 19 |
| | ACCA | | C*+C*+A | | |
| 1808 | CTTACCTTAGTTAAG | A571806Hi | +C*+T*+T*A*C*C*T*T*A*G*T*T*A*A*G*+A* | 49525 | 18 |
| | ACC | | +C*+C | | |
| 1809 | ACTTACCTTAGTTAA | A571807Hi | +A*+C*+T*T*A*C*C*T*T*A*G*T*T*A*A*G*+ | 49525 | 19 |
| | GACC | | A*+C*+C | | |
| 1810 | TACTTACCTTAGTTAA | A571808Hi | +T*+A*+C*T*T*A*C*C*T*T*A*G*T*T*A*A*+ | 49526 | 19 |
| | GAC | | G*+A*+C | | |
| 1811 | AATACTTACCTTAGTT | A571809Hi | +A*+A*+T*A*C*T*T*A*C*C*T*T*A*G*T*T*+ | 49528 | 19 |
| | AAG | | A*+A*+G | | |
| 1812 | ATTTCTGAATCCCAG | A571810H | +A*+T*+T*T*C*T*G*A*A*T*C*C*C*A*G*G*+ | 49596 | 19 |
| | GATT | | A*+T*+T | | |
| 1813 | TAATCCACATTTCTG | A571811H | +T*+A*+A*T*C*C*A*C*A*T*T*T*C*T*G*A*+ | 49604 | 19 |
| | AATC | | A*+T*+C | | |
| 1814 | ACTTTTAATCCACATT | A571812H | +A*+C*+T*T*T*T*A*A*T*C*C*A*C*A*T*T*+ | 49609 | 19 |
| | TCT | | T*+C*+T | | |
| 1815 | TACATCTGCAGTGTG | A571813H | +T*+A*+C*A*T*C*T*G*C*A*G*T*G*T*G*T*+ | 49699 | 19 |
| | TGAA | | G*+A*+A | | |
| 1816 | ATACATCTGCAGTGT | A571814H | +A*+T*+A*C*A*T*C*T*G*C*A*G*T*G*T*G*+ | 49700 | 19 |
| | GTGA | | T*+G*+A | | |
| 1817 | GGATACATCTGCAGT | A571815H | +G*+G*+A*T*A*C*A*T*C*T*G*C*A*G*T*G*+ | 49702 | 19 |
| | GTGT | | T*+G*+T | | |
| 1818 | TCTGGATACATCTGC | A571816H | +T*+C*+T*G*G*A*T*A*C*A*T*C*T*G*C*A*+ | 49705 | 19 |
| | AGTG | | G*+T*+G | | |
| 1819 | ATCTGGATACATCTG | A571817H | +A*+T*+C*T*G*G*A*T*A*C*A*T*C*T*G*C*+ | 49706 | 19 |
| | CAGT | | A*+G*+T | | |
| 1820 | TCATCTGGATACATC | A571818H | +T*+C*+A*T*C*T*G*G*A*T*A*C*A*T*C*T*+ | 49708 | 19 |
| | TGCA | | G*+C*+A | | |
| 1821 | TTGGTCATCTGGATA | A571819H | +T*+T*+G*G*T*C*A*T*C*T*G*G*A*T*A*C*+ | 49712 | 19 |
| | CATC | | A*+T*+C | | |
| 1822 | CTTGGTCATCTGGAT | A571820H | +C*+T*+T*G*G*T*C*A*T*C*T*G*G*A*T*A*+ | 49713 | 19 |
| | ACAT | | C*+A*+T | | |
| 1823 | CTTAAACCCAGACAC | A571821H | +C*+T*+T*A*A*A*C*C*C*A*G*A*C*A*C*T*+ | 49861 | 19 |
| | TGAA | | G*+A*+A | | |
| 1824 | GACTTAAACCCAGAC | A571822H | +G*+A*+C*T*T*A*A*A*C*C*C*A*G*A*C*A*+ | 49863 | 19 |
| | ACTG | | C*+T*+G | | |
| 1825 | CTATAAGACTTAAAC | A571823H | +C*+T*+A*T*A*A*G*A*C*T*T*A*A*A*C*C*+ | 49869 | 19 |
| | CCAG | | C*+A*+G | | |
| 1826 | CCTATAAGACTTAAA | A571824H | +C*+C*+T*A*T*A*A*G*A*C*T*T*A*A*A*C*+ | 49870 | 19 |
| | CCCA | | C*+C*+A | | |
| 1827 | CCTATAAGACTTAAA | A571825H | +C*+C*+T*A*T*A*A*G*A*C*T*T*A*A*A*+C* | 49871 | 18 |
| | CCC | | +C*+C | | |
| 1828 | TCCTATAAGACTTAA | A571826H | +T*+C*+C*T*A*T*A*A*G*A*C*T*T*A*A*A*+ | 49871 | 19 |
| | ACCC | | C*+C*+C | | |
| 1829 | TCCTATAAGACTTAA | A571827H | +T*+C*+C*T*A*T*A*A*G*A*C*T*T*A*A*+A* | 49872 | 18 |
| | ACC | | +C*+C | | |
| 1830 | ATCCTATAAGACTTA | A571828H | +A*+T*+C*C*T*A*T*A*A*G*A*C*T*T*A*A*+ | 49872 | 19 |
| | AACC | | A*+C*+C | | |
| 1831 | GATGTCCAGTTCCTA | A571829H | +G*+A*+T*G*T*C*C*A*G*T*T*C*C*T*+A*+T | 49916 | 17 |
| | TG | | *+G | | |
| 1832 | AGATGTCCAGTTCCT | A571830H | +A*+G*+A*T*G*T*C*C*A*G*T*T*C*C*T*+A* | 49916 | 18 |
| | ATG | | +T*+G | | |
| 1833 | CAGATGTCCAGTTCC | A571831H | +C*+A*+G*A*T*G*T*C*C*A*G*T*T*C*C*T*+ | 49916 | 19 |
| | TATG | | A*+T*+G | | |
| 1834 | CAGATGTCCAGTTCC | A571832H | +C*+A*+G*A*T*G*T*C*C*A*G*T*T*C*C*+T* | 49917 | 18 |
| | TAT | | +A*+T | | |
| 1835 | GCAGATGTCCAGTTC | A571833H | +G*+C*+A*G*A*T*G*T*C*C*A*G*T*T*C*C*+ | 49917 | 19 |
| | CTAT | | T*+A*+T | | |
| 1836 | GCAGATGTCCAGTTC | A571834H | +G*+C*+A*G*A*T*G*T*C*C*A*G*T*T*C*+C* | 49918 | 18 |
| | CTA | | +T*+A | | |
| 1837 | AGCAGATGTCCAGTT | A571835H | +A*+G*+C*A*G*A*T*G*T*C*C*A*G*T*T*C*+ | 49918 | 19 |
| | CCTA | | C*+T*+A | | |
| 1838 | CAGCAGATGTCCAGT | A571836H | +C*+A*+G*C*A*G*A*T*G*T*C*C*A*G*T*T*+ | 49919 | 19 |
| | TCCT | | C*+C*+T | | |
| 1839 | CCAGCAGATGTCCAG | A571837H | +C*+C*+A*G*C*A*G*A*T*G*T*C*C*A*G*T*+ | 49920 | 19 |
| | TTCC | | T*+C*+C | | |
| 1840 | CCAGCAGATGTCCAG | A571838H | +C*+C*+A*G*C*A*G*A*T*G*T*C*C*A*G*+T* | 49921 | 18 |
| | TTC | | +T*+C | | |
| 1841 | GCCAGCAGATGTCCA | A571839H | +G*+C*+C*A*G*C*A*G*A*T*G*T*C*C*A*G*+ | 49921 | 19 |
| | GTTC | | T*+T*+C | | |
| 1842 | GCCAGCAGATGTCCA | A571840H | +G*+C*+C*A*G*C*A*G*A*T*G*T*C*C*A*+G* | 49922 | 18 |
| | GTT | | +T*+T | | |
| 1843 | TGCCAGCAGATGTCC | A571841H | +T*+G*+C*C*A*G*C*A*G*A*T*G*T*C*C*A*+ | 49922 | 19 |
| | AGTT | | G*+T*+T | | |
| 1844 | ATTTTTGCCAGCAGA | A571842H | +A*+T*+T*T*T*T*G*C*C*A*G*C*A*G*A*T*+ | 49927 | 19 |
| | TGTC | | G*+T*+C | | |
| 1845 | AGAATTTTTGCCAGC | A571843H | +A*+G*+A*A*T*T*T*T*T*G*C*C*A*G*C*A*+ | 49930 | 19 |
| | AGAT | | G*+A*+T | | |
| 1846 | AAATCGGTATAAGCT | A571844H | +A*+A*+A*T*C*G*G*T*A*T*A*A*G*C*T*G*+ | 49952 | 19 |
| | GGAA | | G*+A*+A | | |
| 1847 | TAAATCGGTATAAGC | A571845H | +T*+A*+A*A*T*C*G*G*T*A*T*A*A*G*C*T*+ | 49953 | 19 |
| | TGGA | | G*+G*+A | | |
| 1848 | TAAATCGGTATAAGC | A571846H | +T*+A*+A*A*T*C*G*G*T*A*T*A*A*G*C*+T* | 49954 | 18 |
| | TGG | | +G*+G | | |
| 1849 | TTAAATCGGTATAAG | A571847H | +T*+T*+A*A*A*T*C*G*G*T*A*T*A*A*G*C*+ | 49954 | 19 |
| | CTGG | | T*+G*+G | | |
| 1850 | TTAAATCGGTATAAG | A571848H | +T*+T*+A*A*A*T*C*G*G*T*A*T*A*A*G*+C* | 49955 | 18 |
| | CTG | | +T*+G | | |
| 1851 | CTTAAATCGGTATAA | A571849H | +C*+T*+T*A*A*A*T*C*G*G*T*A*T*A*A*G*+ | 49955 | 19 |
| | GCTG | | C*+T*+G | | |
| 1852 | CTTAAATCGGTATAA | A571850H | +C*+T*+T*A*A*A*T*C*G*G*T*A*T*A*A*+G* | 49956 | 18 |
| | GCT | | +C*+T | | |
| 1853 | CCTTAAATCGGTATA | A571851H | +C*+C*+T*T*A*A*A*T*C*G*G*T*A*T*A*A*+ | 49956 | 19 |
| | AGCT | | G*+C*+T | | |
| 1854 | CTTAAATCGGTATAA | A571852H | +C*+T*+T*A*A*A*T*C*G*G*T*A*T*A*+A*+G | 49957 | 17 |
| | GC | | *+C | | |
| 1855 | CCTTAAATCGGTATA | A571853H | +C*+C*+T*T*A*A*A*T*C*G*G*T*A*T*A*+A* | 49957 | 18 |
| | AGC | | +G*+C | | |
| 1856 | TCCTTAAATCGGTAT | A571854H | +T*+C*+C*T*T*A*A*A*T*C*G*G*T*A*T*A*+ | 49957 | 19 |
| | AAGC | | A*+G*+C | | |
| 1857 | TCCTTAAATCGGTAT | A571855H | +T*+C*+C*T*T*A*A*A*T*C*G*G*T*A*T*+A* | 49958 | 18 |
| | AAG | | +A*+G | | |
| 1858 | ATCCTTAAATCGGTA | A571856H | +A*+T*+C*C*T*T*A*A*A*T*C*G*G*T*A*T*+ | 49958 | 19 |
| | TAAG | | A*+A*+G | | |
| 1859 | CATCCTTAAATCGGT | A571857H | +C*+A*+T*C*C*T*T*A*A*A*T*C*G*G*T*A*+ | 49959 | 19 |
| | ATAA | | T*+A*+A | | |
| 1860 | ACATCCTTAAATCGG | A571858H | +A*+C*+A*T*C*C*T*T*A*A*A*T*C*G*G*T*+ | 49960 | 19 |
| | TATA | | A*+T*+A | | |
| 1861 | ACATCCTTAAATCGG | A571859H | +A*+C*+A*T*C*C*T*T*A*A*A*T*C*G*G*+T* | 49961 | 18 |
| | TAT | | +A*+T | | |
| 1862 | AACATCCTTAAATCG | A571860H | +A*+A*+C*A*T*C*C*T*T*A*A*A*T*C*G*G*+ | 49961 | 19 |
| | GTAT | | T*+A*+T | | |
| 1863 | AACATCCTTAAATCG | A571861H | +A*+A*+C*A*T*C*C*T*T*A*A*A*T*C*G*+G* | 49962 | 18 |
| | GTA | | +T*+A | | |
| 1864 | CAACATCCTTAAATC | A571862H | +C*+A*+A*C*A*T*C*C*T*T*A*A*A*T*C*G*+ | 49962 | 19 |
| | GGTA | | G*+T*+A | | |
| 1865 | GCAACATCCTTAAAT | A571863H | +G*+C*+A*A*C*A*T*C*C*T*T*A*A*A*T*C*+ | 49963 | 19 |
| | CGGT | | G*+G*+T | | |
| 1866 | GCAACATCCTTAAAT | A571864H | +G*+C*+A*A*C*A*T*C*C*T*T*A*A*A*T*+C* | 49964 | 18 |
| | CGG | | +G*+G | | |
| 1867 | AGCAACATCCTTAAA | A571865H | +A*+G*+C*A*A*C*A*T*C*C*T*T*A*A*A*T*+ | 49964 | 19 |
| | TCGG | | C*+G*+G | | |
| 1868 | CAGCAACATCCTTAA | A571866H | +C*+A*+G*C*A*A*C*A*T*C*C*T*T*A*A*A*+ | 49965 | 19 |
| | ATCG | | T*+C*+G | | |
| 1869 | TTCAGCAACATCCTT | A571867H | +T*+T*+C*A*G*C*A*A*C*A*T*C*C*T*T*A*+ | 49967 | 19 |
| | AAAT | | A*+A*+T | | |
| 1870 | CCAAGATAATGCACA | A571868Hi | +C*+C*+A*A*G*A*T*A*A*T*G*C*A*C*A*+T* | 49998 | 18 |
| | TAC | | +A*+C | | |
| 1871 | CTTTAGAGATTCAAT | A571869Hi | +C*+T*+T*T*A*G*A*G*A*T*T*C*A*A*T*C*+ | 50151 | 19 |
| | CCAA | | C*+A*+A | | |
| 1872 | ACACTGGATCTTTAG | A571870Hi | +A*+C*+A*C*T*G*G*A*T*C*T*T*T*A*G*A*+ | 50160 | 19 |
| | AGAT | | G*+A*+T | | |
| 1873 | ATGCTGACTTTAGAG | A571871Hi | +A*+T*+G*C*T*G*A*C*T*T*T*A*G*A*G*A*+ | 50188 | 19 |
| | ATTT | | T*+T*+T | | |
| 1874 | CATGCTGACTTTAGA | A571872Hi | +C*+A*+T*G*C*T*G*A*C*T*T*T*A*G*A*G*+ | 50189 | 19 |
| | GATT | | A*+T*+T | | |
| 1875 | TTTATGAAAGTTGCA | A571873Hi | +T*+T*+T*A*T*G*A*A*A*G*T*T*G*C*A*A*+ | 50587 | 19 |
| | AACT | | A*+C*+T | | |
| 1876 | TAGTTTATGAAAGTT | A571874Hi | +T*+A*+G*T*T*T*A*T*G*A*A*A*G*T*T*G*+ | 50590 | 19 |
| | GCAA | | C*+A*+A | | |
| 1877 | CTGGTCAATGCTGCC | A571875Hi | +C*+T*+G*G*T*C*A*A*T*G*C*T*G*C*C*A*+ | 50937 | 19 |
| | AGTT | | G*+T*+T | | |
| 1878 | CCTGGTCAATGCTGC | A571876Hi | +C*+C*+T*G*G*T*C*A*A*T*G*C*T*G*C*C*+ | 50938 | 19 |
| | CAGT | | A*+G*+T | | |
| 1879 | TTCCTGGTCAATGCT | A571877Hi | +T*+T*+C*C*T*G*G*T*C*A*A*T*G*C*T*G*+ | 50940 | 19 |
| | GCCA | | C*+C*+A | | |
| 1880 | TTCCTGGTCAATGCT | A571878Hi | +T*+T*+C*C*T*G*G*T*C*A*A*T*G*C*T*+G* | 50941 | 18 |
| | GCC | | +C*+C | | |
| 1881 | TATTCCTGGTCAATG | A571879Hi | +T*+A*+T*T*C*C*T*G*G*T*C*A*A*T*G*C*+ | 50942 | 19 |
| | CTGC | | T*+G*+C | | |
| 1882 | ATTCCTGGTCAATGC | A571880Hi | +A*+T*+T*C*C*T*G*G*T*C*A*A*T*G*C*+T* | 50942 | 18 |
| | TGC | | +G*+C | | |
| 1883 | CTGTGCAAAGATTAG | A571881Hi | +C*+T*+G*T*G*C*A*A*A*G*A*T*T*A*G*A*+ | 50971 | 19 |
| | AGAC | | G*+A*+C | | |
| 1884 | CCTGAGGCACATTAT | A571882Hi | +C*+C*+T*G*A*G*G*C*A*C*A*T*T*A*T*G*+ | 51097 | 19 |
| | GAAA | | A*+A*+A | | |
| 1885 | AATTTATGTATGTCT | A571883Hi | +A*+A*+T*T*T*A*T*G*T*A*T*G*T*C*T*G*+ | 51727 | 19 |
| | GAGG | | A*+G*+G | | |
| 1886 | AATTTATGTATGTCT | A571884Hi | +A*+A*+T*T*T*A*T*G*T*A*T*G*T*C*T*+G* | 51728 | 18 |
| | GAG | | +A*+G | | |
| 1887 | GATAAGCAAAGTAGC | A571885Hi | +G*+A*+T*A*A*G*C*A*A*A*G*T*A*G*C*C*+ | 51745 | 19 |
| | CTTA | | T*+T*+A | | |
| 1888 | TGATAAGCAAAGTAG | A571886Hi | +T*+G*+A*T*A*A*G*C*A*A*A*G*T*A*G*C*+ | 51746 | 19 |
| | CCTT | | C*+T*+T | | |
| 1889 | TTGATAAGCAAAGTA | A571887Hi | +T*+T*+G*A*T*A*A*G*C*A*A*A*G*T*A*G*+ | 51747 | 19 |
| | GCCT | | C*+C*+T | | |
| 1890 | TTTGATAAGCAAAGT | A571888Hi | +T*+T*+T*G*A*T*A*A*G*C*A*A*A*G*T*A*+ | 51748 | 19 |
| | AGCC | | G*+C*+C | | |
| 1891 | ATACTAATATTTGATA | A571889Hi | +A*+T*+A*C*T*A*A*T*A*T*T*T*G*A*T*A*+ | 51757 | 19 |
| | AGC | | A*+G*+C | | |
| 1892 | AACCACCTTAAGCAC | A571890Hi | +A*+A*+C*C*A*C*C*T*T*A*A*G*C*A*C*A*+ | 52275 | 19 |
| | ATCC | | T*+C*+C | | |
| 1893 | ACCACCTTAAGCACA | A571891Hi | +A*+C*+C*A*C*C*T*T*A*A*G*C*A*C*A*+T* | 52275 | 18 |
| | TCC | | +C*+C | | |
| 1894 | GGTTCTCTAGAGAGC | A571892Hi | +G*+G*+T*T*C*T*C*T*A*G*A*G*A*G*C*C*+ | 52461 | 19 |
| | CTAG | | T*+A*+G | | |
| 1895 | AGTAGGTTCTCTAGA | A571893Hi | +A*+G*+T*A*G*G*T*T*C*T*C*T*A*G*A*+G* | 52466 | 18 |
| | GAG | | +A*+G | | |
| 1896 | GATATTAAACAGGAT | A571894Hi | +G*+A*+T*A*T*T*A*A*A*C*A*G*G*A*T*T*+ | 52554 | 19 |
| | TAAA | | A*+A*+A | | |
| 1897 | AGATATTAAACAGGA | A571895Hi | +A*+G*+A*T*A*T*T*A*A*A*C*A*G*G*A*T*+ | 52555 | 19 |
| | TTAA | | T*+A*+A | | |
| 1898 | GCTTGTCTAAAGCCA | A571896Hi | +G*+C*+T*T*G*T*C*T*A*A*A*G*C*C*A*+T* | 52590 | 18 |
| | TAG | | +A*+G | | |
| 1899 | TTTCAAAGATGCTTG | A571897Hi | +T*+T*+T*C*A*A*A*G*A*T*G*C*T*T*G*T*+ | 52599 | 19 |
| | TCTA | | C*+T*+A | | |
| 1900 | CTCTCTCTCATGACT | A571898Hi | +C*+T*+C*T*C*T*C*T*C*A*T*G*A*C*T*A*+ | 52705 | 19 |
| | AAGA | | A*+G*+A | | |
| 1901 | GTTCATTTAACTATA | A571899Hi | +G*+T*+T*C*A*T*T*T*A*A*C*T*A*T*A*G*+ | 52772 | 19 |
| | GATA | | A*+T*+A | | |
| 1902 | AGGATGTTCATTTAA | A571900Hi | +A*+G*+G*A*T*G*T*T*C*A*T*T*T*A*A*C*+ | 52777 | 19 |
| | CTAT | | T*+A*+T | | |
| 1903 | TAGGATGTTCATTTA | A571901Hi | +T*+A*+G*G*A*T*G*T*T*C*A*T*T*T*A*A*+ | 52778 | 19 |
| | ACTA | | C*+T*+A | | |
| 1904 | CTAGGATGTTCATTT | A571902Hi | +C*+T*+A*G*G*A*T*G*T*T*C*A*T*T*T*+A* | 52780 | 18 |
| | AAC | | +A*+C | | |
| 1905 | ATACCTAGGATGTTC | A571903Hi | +A*+T*+A*C*C*T*A*G*G*A*T*G*T*T*C*A*+ | 52783 | 19 |
| | ATTT | | T*+T*+T | | |
| 1906 | CAATACCTAGGATGT | A571904Hi | +C*+A*+A*T*A*C*C*T*A*G*G*A*T*G*T*+T* | 52786 | 18 |
| | TCA | | +C*+A | | |
| 1907 | GAAACAATACCTAGG | A571905Hi | +G*+A*+A*A*C*A*A*T*A*C*C*T*A*G*G*+A* | 52790 | 18 |
| | ATG | | +T*+G | | |
| 1908 | ATGAAACAATACCTA | A571906Hi | +A*+T*+G*A*A*A*C*A*A*T*A*C*C*T*A*G*+ | 52791 | 19 |
| | GGAT | | G*+A*+T | | |
| 1909 | TATATGAAACAATAC | A571907Hi | +T*+A*+T*A*T*G*A*A*A*C*A*A*T*A*C*C*+ | 52794 | 19 |
| | CTAG | | T*+A*+G | | |
| 1910 | TGTATATGAAACAAT | A571908Hi | +T*+G*+T*A*T*A*T*G*A*A*A*C*A*A*T*A*+ | 52796 | 19 |
| | ACCT | | C*+C*+T | | |
| 1911 | TTTGTATATGAAACA | A571909Hi | +T*+T*+T*G*T*A*T*A*T*G*A*A*A*C*A*A*+ | 52798 | 19 |
| | ATAC | | T*+A*+C | | |
| 1912 | GAGTTTGTATATGAA | A571910Hi | +G*+A*+G*T*T*T*G*T*A*T*A*T*G*A*A*A*+ | 52801 | 19 |
| | ACAA | | C*+A*+A | | |
| 1913 | AACAGTATAAAGCAG | A571911Hi | +A*+A*+C*A*G*T*A*T*A*A*A*G*C*A*G*A*+ | 52819 | 19 |
| | AGAG | | G*+A*+G | | |
| 1914 | CTAAGATTGTCTGAA | A571912H | +C*+T*+A*A*G*A*T*T*G*T*C*T*G*A*A*A*+ | 52892 | 19 |
| | ATCC | | T*+C*+C | | |
| 1915 | GCTAAGATTGTCTGA | A571913H | +G*+C*+T*A*A*G*A*T*T*G*T*C*T*G*A*A*+ | 52893 | 19 |
| | AATC | | A*+T*+C | | |
| 1916 | TTGCTAAGATTGTCT | A571914H | +T*+T*+G*C*T*A*A*G*A*T*T*G*T*C*T*G*+ | 52895 | 19 |
| | GAAA | | A*+A*+A | | |
| 1917 | ATTGCTAAGATTGTC | A571915H | +A*+T*+T*G*C*T*A*A*G*A*T*T*G*T*C*T*+ | 52896 | 19 |
| | TGAA | | G*+A*+A | | |
| 1918 | GATTGCTAAGATTGT | A571916H | +G*+A*+T*T*G*C*T*A*A*G*A*T*T*G*T*C*+ | 52897 | 19 |
| | CTGA | | T*+G*+A | | |
| 1919 | AGGATTGCTAAGATT | A571917H | +A*+G*+G*A*T*T*G*C*T*A*A*G*A*T*T*G*+ | 52899 | 19 |
| | GTCT | | T*+C*+T | | |
| 1920 | AGGATTGCTAAGATT | A571918H | +A*+G*+G*A*T*T*G*C*T*A*A*G*A*T*T*+G* | 52900 | 18 |
| | GTC | | +T*+C | | |
| 1921 | GAGGATTGCTAAGAT | A571919H | +G*+A*+G*G*A*T*T*G*C*T*A*A*G*A*T*T*+ | 52900 | 19 |
| | TGTC | | G*+T*+C | | |
| 1922 | TGACAATTTGAGGAT | A571920H | +T*+G*+A*C*A*A*T*T*T*G*A*G*G*A*T*T*+ | 52909 | 19 |
| | TGCT | | G*+C*+T | | |
| 1923 | TGACAATTTGAGGAT | A571921H | +T*+G*+A*C*A*A*T*T*T*G*A*G*G*A*T*+T* | 52910 | 18 |
| | TGC | | +G*+C | | |
| 1924 | CTGACAATTTGAGGA | A571922H | +C*+T*+G*A*C*A*A*T*T*T*G*A*G*G*A*T*+ | 52910 | 19 |
| | TTGC | | T*+G*+C | | |
| 1925 | GCTGACAATTTGAGG | A571923H | +G*+C*+T*G*A*C*A*A*T*T*T*G*A*G*G*A*+ | 52911 | 19 |
| | ATTG | | T*+T*+G | | |
| 1926 | GCTGACAATTTGAGG | A571924H | +G*+C*+T*G*A*C*A*A*T*T*T*G*A*G*G*+A* | 52912 | 18 |
| | ATT | | +T*+T | | |
| 1927 | TGCTGACAATTTGAG | A571925H | +T*+G*+C*T*G*A*C*A*A*T*T*T*G*A*G*G*+ | 52912 | 19 |
| | GATT | | A*+T*+T | | |
| 1928 | ATGCTGACAATTTGA | A571926H | +A*+T*+G*C*T*G*A*C*A*A*T*T*T*G*A*G*+ | 52913 | 19 |
| | GGAT | | G*+A*+T | | |
| 1929 | GATGCTGACAATTTG | A571927H | +G*+A*+T*G*C*T*G*A*C*A*A*T*T*T*G*A*+ | 52914 | 19 |
| | AGGA | | G*+G*+A | | |
| 1930 | GATGCTGACAATTTG | A571928H | +G*+A*+T*G*C*T*G*A*C*A*A*T*T*T*G*+A* | 52915 | 18 |
| | AGG | | +G*+G | | |
| 1931 | AGATGCTGACAATTT | A571929H | +A*+G*+A*T*G*C*T*G*A*C*A*A*T*T*T*G*+ | 52915 | 19 |
| | GAGG | | A*+G*+G | | |
| 1932 | AGATGCTGACAATTT | A571930H | +A*+G*+A*T*G*C*T*G*A*C*A*A*T*T*T*+G* | 52916 | 18 |
| | GAG | | +A*+G | | |
| 1933 | AAGATGCTGACAATT | A571931H | +A*+A*+G*A*T*G*C*T*G*A*C*A*A*T*T*T*+ | 52916 | 19 |
| | TGAG | | G*+A*+G | | |
| 1934 | TAGAAAAAGATGCTG | A571932H | +T*+A*+G*A*A*A*A*A*G*A*T*G*C*T*G*A*+ | 52922 | 19 |
| | ACAA | | C*+A*+A | | |
| 1935 | TTAGAAAAAGATGCT | A571933H | +T*+T*+A*G*A*A*A*A*A*G*A*T*G*C*T*G*+ | 52923 | 19 |
| | GACA | | A*+C*+A | | |
| 1936 | TTAGAAAAAGATGCT | A571934H | +T*+T*+A*G*A*A*A*A*A*G*A*T*G*C*T*+G* | 52924 | 18 |
| | GAC | | +A*+C | | |
| 1937 | CTTAGAAAAAGATGC | A571935H | +C*+T*+T*A*G*A*A*A*A*A*G*A*T*G*C*T*+ | 52924 | 19 |
| | TGAC | | G*+A*+C | | |
| 1938 | GCTTAGAAAAAGATG | A571936H | +G*+C*+T*T*A*G*A*A*A*A*A*G*A*T*G*C*+ | 52925 | 19 |
| | CTGA | | T*+G*+A | | |
| 1939 | ACCAGCAGCTTAGAA | A571937H | +A*+C*+C*A*G*C*A*G*C*T*T*A*G*A*A*A*+ | 52932 | 19 |
| | AAAG | | A*+A*+G | | |
| 1940 | GCACCAGCAGCTTAG | A571938H | +G*+C*+A*C*C*A*G*C*A*G*C*T*T*A*G*A*+ | 52934 | 19 |
| | AAAA | | A*+A*+A | | |
| 1941 | TGCACCAGCAGCTTA | A571939H | +T*+G*+C*A*C*C*A*G*C*A*G*C*T*T*A*G*+ | 52935 | 19 |
| | GAAA | | A*+A*+A | | |
| 1942 | GATGCACCAGCAGCT | A571940H | +G*+A*+T*G*C*A*C*C*A*G*C*A*G*C*T*T*+ | 52937 | 19 |
| | TAGA | | A*+G*+A | | |
| 1943 | GATCCTTTTGTTCCAT | A571941H | +G*+A*+T*C*C*T*T*T*T*G*T*T*C*C*A*T*+ | 52997 | 19 |
| | GAT | | G*+A*+T | | |
| 1944 | CTGTTGATCCTTTTGT | A571942H | +C*+T*+G*T*T*G*A*T*C*C*T*T*T*T*G*T*+ | 53002 | 19 |
| | TCC | | T*+C*+C | | |
| 1945 | CATAACATTTAGGAT | A571943Hi | +C*+A*+T*A*A*C*A*T*T*T*A*G*G*A*T*G*+ | 53037 | 19 |
| | GCAA | | C*+A*+A | | |
| 1946 | AGAGTCAGATAAATA | A571944Hi | +A*+G*+A*G*T*C*A*G*A*T*A*A*A*T*A*C*+ | 53052 | 19 |
| | CATA | | A*+T*+A | | |
| 1947 | TAGAGTCAGATAAAT | A571945Hi | +T*+A*+G*A*G*T*C*A*G*A*T*A*A*A*T*A*+ | 53053 | 19 |
| | ACAT | | C*+A*+T | | |
| 1948 | TTGCAACAGAGGTTT | A571946H | +T*+T*+G*C*A*A*C*A*G*A*G*G*T*T*T*A*+ | 53096 | 19 |
| | AGAA | | G*+A*+A | | |
| 1949 | ACTTGCAACAGAGGT | A571947H | +A*+C*+T*T*G*C*A*A*C*A*G*A*G*G*T*T*+ | 53098 | 19 |
| | TTAG | | T*+A*+G | | |
| 1950 | AAACACTTGCAACAG | A571948H | +A*+A*+A*C*A*C*T*T*G*C*A*A*C*A*G*A*+ | 53102 | 19 |
| | AGGT | | G*+G*+T | | |
| 1951 | TTTGCAAAACACTTG | A571949H | +T*+T*+T*G*C*A*A*A*A*C*A*C*T*T*G*C*+ | 53108 | 19 |
| | CAAC | | A*+A*+C | | |
| 1952 | TTTTGCAAAACACTT | A571950H | +T*+T*+T*T*G*C*A*A*A*A*C*A*C*T*T*G*+ | 53109 | 19 |
| | GCAA | | C*+A*+A | | |
| 1953 | CTTTTGCAAAACACT | A571951H | +C*+T*+T*T*T*G*C*A*A*A*A*C*A*C*T*T*+ | 53110 | 19 |
| | TGCA | | G*+C*+A | | |
| 1954 | ATAGCTACTTTTGCA | A571952H | +A*+T*+A*G*C*T*A*C*T*T*T*T*G*C*A*A*+ | 53117 | 19 |
| | AAAC | | A*+A*+C | | |
| 1955 | CCATAGCTACTTTTG | A571953H | +C*+C*+A*T*A*G*C*T*A*C*T*T*T*T*G*C*+ | 53119 | 19 |
| | CAAA | | A*+A*+A | | |
| 1956 | TCCATAGCTACTTTT | A571954H | +T*+C*+C*A*T*A*G*C*T*A*C*T*T*T*T*G*+ | 53120 | 19 |
| | GCAA | | C*+A*+A | | |
| 1957 | ACACGCTCTCTCTAG | A571955H | +A*+C*+A*C*G*C*T*C*T*C*T*C*T*A*G*C*+ | 53157 | 19 |
| | CATC | | A*+T*+C | | |
| 1958 | CACACGCTCTCTCTA | A571956H | +C*+A*+C*A*C*G*C*T*C*T*C*T*C*T*A*G*+ | 53158 | 19 |
| | GCAT | | C*+A*+T | | |
| 1959 | CACACGCTCTCTCTA | A571957H | +C*+A*+C*A*C*G*C*T*C*T*C*T*C*T*A*+G* | 53159 | 18 |
| | GCA | | +C*+A | | |
| 1960 | TCACACGCTCTCTCT | A571958H | +T*+C*+A*C*A*C*G*C*T*C*T*C*T*C*T*A*+ | 53159 | 19 |
| | AGCA | | G*+C*+A | | |
| 1961 | TCACACGCTCTCTCT | A571959H | +T*+C*+A*C*A*C*G*C*T*C*T*C*T*C*T*+A* | 53160 | 18 |
| | AGC | | +G*+C | | |
| 1962 | ATCACACGCTCTCTC | A571960H | +A*+T*+C*A*C*A*C*G*C*T*C*T*C*T*C*T*+ | 53160 | 19 |
| | TAGC | | A*+G*+C | | |
| 1963 | GATCACACGCTCTCT | A571961H | +G*+A*+T*C*A*C*A*C*G*C*T*C*T*C*T*C*+ | 53161 | 19 |
| | CTAG | | T*+A*+G | | |
| 1964 | GATCACACGCTCTCT | A571962H | +G*+A*+T*C*A*C*A*C*G*C*T*C*T*C*T*+C* | 53162 | 18 |
| | CTA | | +T*+A | | |
| 1965 | TGATCACACGCTCTC | A571963H | +T*+G*+A*T*C*A*C*A*C*G*C*T*C*T*C*T*+ | 53162 | 19 |
| | TCTA | | C*+T*+A | | |
| 1966 | CTGATCACACGCTCT | A571964H | +C*+T*+G*A*T*C*A*C*A*C*G*C*T*C*T*C*+ | 53163 | 19 |
| | CTCT | | T*+C*+T | | |
| 1967 | TCTGATCACACGCTC | A571965H | +T*+C*+T*G*A*T*C*A*C*A*C*G*C*T*C*T*+ | 53164 | 19 |
| | TCTC | | C*+T*+C | | |
| 1968 | TCTGATCACACGCTC | A571966H | +T*+C*+T*G*A*T*C*A*C*A*C*G*C*T*C*+T* | 53165 | 18 |
| | TCT | | +C*+T | | |
| 1969 | TTCTGATCACACGCT | A571967H | +T*+T*+C*T*G*A*T*C*A*C*A*C*G*C*T*C*+ | 53165 | 19 |
| | CTCT | | T*+C*+T | | |
| 1970 | ATTCTGATCACACGC | A571968H | +A*+T*+T*C*T*G*A*T*C*A*C*A*C*G*C*T*+ | 53166 | 19 |
| | TCTC | | C*+T*+C | | |
| 1971 | TATTCTGATCACACG | A571969H | +T*+A*+T*T*C*T*G*A*T*C*A*C*A*C*G*C*+ | 53167 | 19 |
| | CTCT | | T*+C*+T | | |
| 1972 | TATTCTGATCACACG | A571970H | +T*+A*+T*T*C*T*G*A*T*C*A*C*A*C*G*+C* | 53168 | 18 |
| | CTC | | +T*+C | | |
| 1973 | TTATTCTGATCACAC | A571971H | +T*+T*+A*T*T*C*T*G*A*T*C*A*C*A*C*G*+ | 53168 | 19 |
| | GCTC | | C*+T*+C | | |
| 1974 | TTATTCTGATCACAC | A571972H | +T*+T*+A*T*T*C*T*G*A*T*C*A*C*A*C*+G* | 53169 | 18 |
| | GCT | | +C*+T | | |
| 1975 | GTTATTCTGATCACA | A571973H | +G*+T*+T*A*T*T*C*T*G*A*T*C*A*C*A*C*+ | 53169 | 19 |
| | CGCT | | G*+C*+T | | |
| 1976 | CTGTTATTCTGATCA | A571974H | +C*+T*+G*T*T*A*T*T*C*T*G*A*T*C*A*C*+ | 53171 | 19 |
| | CACG | | A*+C*+G | | |
| 1977 | CATGATGCTGTTATT | A571975H | +C*+A*+T*G*A*T*G*C*T*G*T*T*A*T*T*C*+ | 53178 | 19 |
| | CTGA | | T*+G*+A | | |
| 1978 | CAACCATGATGCTGT | A571976H | +C*+A*+A*C*C*A*T*G*A*T*G*C*T*G*T*T*+ | 53182 | 19 |
| | TATT | | A*+T*+T | | |
| 1979 | TCAACCATGATGCTG | A571977H | +T*+C*+A*A*C*C*A*T*G*A*T*G*C*T*G*T*+ | 53183 | 19 |
| | TTAT | | T*+A*+T | | |
| 1980 | TTCAACCATGATGCT | A571978H | +T*+T*+C*A*A*C*C*A*T*G*A*T*G*C*T*G*+ | 53184 | 19 |
| | GTTA | | T*+T*+A | | |
| 1981 | ATTCAACCATGATGC | A571979H | +A*+T*+T*C*A*A*C*C*A*T*G*A*T*G*C*T*+ | 53185 | 19 |
| | TGTT | | G*+T*+T | | |
| 1982 | GCATTCAACCATGAT | A571980H | +G*+C*+A*T*T*C*A*A*C*C*A*T*G*A*T*G*+ | 53187 | 19 |
| | GCTG | | C*+T*+G | | |
| 1983 | AGCATTCAACCATGA | A571981H | +A*+G*+C*A*T*T*C*A*A*C*C*A*T*G*A*T*+ | 53188 | 19 |
| | TGCT | | G*+C*+T | | |
| 1984 | GCAAGCATTCAACCA | A571982H | +G*+C*+A*A*G*C*A*T*T*C*A*A*C*C*A*T*+ | 53191 | 19 |
| | TGAT | | G*+A*+T | | |
| 1985 | AGCAAGCATTCAACC | A571983H | +A*+G*+C*A*A*G*C*A*T*T*C*A*A*C*C*A*+ | 53192 | 19 |
| | ATGA | | T*+G*+A | | |
| 1986 | AAGCAAGCATTCAAC | A571984H | +A*+A*+G*C*A*A*G*C*A*T*T*C*A*A*C*C*+ | 53193 | 19 |
| | CATG | | A*+T*+G | | |
| 1987 | CTTGATTGGCATCTG | A571985H | +C*+T*+T*G*A*T*T*G*G*C*A*T*C*T*G*C*+ | 53218 | 19 |
| | CTCC | | T*+C*+C | | |
| 1988 | GCTTGATTGGCATCT | A571986H | +G*+C*+T*T*G*A*T*T*G*G*C*A*T*C*T*G*+ | 53219 | 19 |
| | GCTC | | C*+T*+C | | |
| 1989 | GCTTGATTGGCATCT | A571987H | +G*+C*+T*T*G*A*T*T*G*G*C*A*T*C*T*+G* | 53220 | 18 |
| | GCT | | +C*+T | | |
| 1990 | TGCTTGATTGGCATC | A571988H | +T*+G*+C*T*T*G*A*T*T*G*G*C*A*T*C*T*+ | 53220 | 19 |
| | TGCT | | G*+C*+T | | |
| 1991 | CTTTGCTTGATTGGC | A571989H | +C*+T*+T*T*G*C*T*T*G*A*T*T*G*G*C*A*+ | 53223 | 19 |
| | ATCT | | T*+C*+T | | |
| 1992 | CTTTGCTTGATTGGC | A571990H | +C*+T*+T*T*G*C*T*T*G*A*T*T*G*G*C*+A* | 53224 | 18 |
| | ATC | | +T*+C | | |
| 1993 | CCTTTGCTTGATTGG | A571991H | +C*+C*+T*T*T*G*C*T*T*G*A*T*T*G*G*C*+ | 53224 | 19 |
| | CATC | | A*+T*+C | | |
| 1994 | TCCTTTGCTTGATTG | A571992H | +T*+C*+C*T*T*T*G*C*T*T*G*A*T*T*G*G*+ | 53225 | 19 |
| | GCAT | | C*+A*+T | | |
| 1995 | TCCTTTGCTTGATTG | A571993H | +T*+C*+C*T*T*T*G*C*T*T*G*A*T*T*G*+G* | 53226 | 18 |
| | GCA | | +C*+A | | |
| 1996 | CTCCTTTGCTTGATT | A571994H | +C*+T*+C*C*T*T*T*G*C*T*T*G*A*T*T*G*+ | 53226 | 19 |
| | GGCA | | G*+C*+A | | |
| 1997 | TCCCTCCTTTGCTTG | A571995H | +T*+C*+C*C*T*C*C*T*T*T*G*C*T*T*G*A*+ | 53229 | 19 |
| | ATTG | | T*+T*+G | | |
| 1998 | TAGTGAAGAATAATA | A571996Hi | +T*+A*+G*T*G*A*A*G*A*A*T*A*A*T*A*T*+ | 53350 | 19 |
| | TGAT | | G*+A*+T | | |
| 1999 | GTAGTGAAGAATAAT | A571997Hi | +G*+T*+A*G*T*G*A*A*G*A*A*T*A*A*T*A*+ | 53351 | 19 |
| | ATGA | | T*+G*+A | | |
| 2000 | CTATTACTTCTGGAA | A571998Hi | +C*+T*+A*T*T*A*C*T*T*C*T*G*G*A*A*T*+ | 53419 | 19 |
| | TTCT | | T*+C*+T | | |
| 2001 | ATGAAATGAAAGATT | A571999Hi | +A*+T*+G*A*A*A*T*G*A*A*A*G*A*T*T*A*+ | 53458 | 19 |
| | AGAC | | G*+A*+C | | |
| 2002 | TAGAGATGCCTACTA | A572000Hi | +T*+A*+G*A*G*A*T*G*C*C*T*A*C*T*A*+A* | 53524 | 18 |
| | AGG | | +G*+G | | |
| 2003 | AAGTTGTAGAGATGC | A572001Hi | +A*+A*+G*T*T*G*T*A*G*A*G*A*T*G*C*+C* | 53530 | 18 |
| | CTA | | +T*+A | | |
| 2004 | GTGGAGTGTATCTGG | A572002Hi | +G*+T*+G*G*A*G*T*G*T*A*T*C*T*G*G*+A* | 53550 | 18 |
| | AGA | | +G*+A | | |
| 2005 | TGTGGAGTGTATCTG | A572003Hi | +T*+G*+T*G*G*A*G*T*G*T*A*T*C*T*G*+G* | 53551 | 18 |
| | GAG | | +A*+G | | |
| 2006 | AACTATGTGTTATGT | A572004Hi | +A*+A*+C*T*A*T*G*T*G*T*T*A*T*G*T*G*+ | 53562 | 19 |
| | GGAG | | G*+A*+G | | |
| 2007 | GAACTATGTGTTATG | A572005Hi | +G*+A*+A*C*T*A*T*G*T*G*T*T*A*T*G*+T* | 53564 | 18 |
| | TGG | | +G*+G | | |
| 2008 | CTGAATGCTAGTATA | A572006Hi | +C*+T*+G*A*A*T*G*C*T*A*G*T*A*T*A*A*+ | 54093 | 19 |
| | AACT | | A*+C*+T | | |
| 2009 | GCTACCTGAATGCTA | A572007Hi | +G*+C*+T*A*C*C*T*G*A*A*T*G*C*T*+A*+G | 54100 | 17 |
| | GT | | *+T | | |
| 2010 | TAGTGCTACCTGAAT | A572008Hi | +T*+A*+G*T*G*C*T*A*C*C*T*G*A*A*T*+G* | 54103 | 18 |
| | GCT | | +C*+T | | |
| 2011 | TAGACATAGTGCTAC | A572009Hi | +T*+A*+G*A*C*A*T*A*G*T*G*C*T*A*C*+C* | 54109 | 18 |
| | CTG | | +T*+G | | |
| 2012 | AACTTAATCTTAACA | A572010Hi | +A*+A*+C*T*T*A*A*T*C*T*T*A*A*C*A*G*+ | 54300 | 19 |
| | GTCA | | T*+C*+A | | |
| 2013 | TAACTTAATCTTAACA | A572011Hi | +T*+A*+A*C*T*T*A*A*T*C*T*T*A*A*C*A*+ | 54301 | 19 |
| | GTC | | G*+T*+C | | |
| 2014 | AATTAGATTGTTTAT | A572012Hi | +A*+A*+T*T*A*G*A*T*T*G*T*T*T*A*T*G*+ | 54395 | 19 |
| | GTGA | | T*+G*+A | | |
| 2015 | GTATTAAATTATTGAT | A572013Hi | +G*+T*+A*T*T*A*A*A*T*T*A*T*T*G*A*T*+ | 54493 | 19 |
| | GGA | | G*+G*+A | | |
| 2016 | ATGCAATAATCTGCA | A572014Hi | +A*+T*+G*C*A*A*T*A*A*T*C*T*G*C*A*T*+ | 54531 | 19 |
| | TGCA | | G*+C*+A | | |
| 2017 | TAATGCAATAATCTG | A572015Hi | +T*+A*+A*T*G*C*A*A*T*A*A*T*C*T*G*C*+ | 54533 | 19 |
| | CATG | | A*+T*+G | | |
| 2018 | CTTAATGTGGACTTA | A572016Hi | +C*+T*+T*A*A*T*G*T*G*G*A*C*T*T*A*+G* | 54594 | 18 |
| | GAA | | +A*+A | | |
| 2019 | TCCTTAATGTGGACT | A572017Hi | +T*+C*+C*T*T*A*A*T*G*T*G*G*A*C*T*+T* | 54596 | 18 |
| | TAG | | +A*+G | | |
| 2020 | TGTTTCCTTAATGTG | A572018Hi | +T*+G*+T*T*T*C*C*T*T*A*A*T*G*T*G*G*+ | 54599 | 19 |
| | GACT | | A*+C*+T | | |
| 2021 | GTTGTTTCCTTAATGT | A572019Hi | +G*+T*+T*G*T*T*T*C*C*T*T*A*A*T*G*T*+ | 54601 | 19 |
| | GGA | | G*+G*+A | | |
| 2022 | TGTTGTTTCCTTAATG | A572020Hi | +T*+G*+T*T*G*T*T*T*C*C*T*T*A*A*T*G*+ | 54602 | 19 |
| | TGG | | T*+G*+G | | |
| 2023 | GGTATGTTGTTTCCT | A572021Hi | +G*+G*+T*A*T*G*T*T*G*T*T*T*C*C*T*T*+ | 54606 | 19 |
| | TAAT | | A*+A*+T | | |
| 2024 | CTAATACATAATCCTT | A572022Hi | +C*+T*+A*A*T*A*C*A*T*A*A*T*C*C*T*+T* | 54799 | 18 |
| | GG | | +G*+G | | |
| 2025 | CACAAGATTTAATCA | A572023Hi | +C*+A*+C*A*A*G*A*T*T*T*A*A*T*C*A*G*+ | 54871 | 19 |
| | GCAG | | C*+A*+G | | |
| 2026 | ATCCACAAGATTTAA | A572024Hi | +A*+T*+C*C*A*C*A*A*G*A*T*T*T*A*A*+T* | 54875 | 18 |
| | TCA | | +C*+A | | |
| 2027 | CCACTGTGATTTGAT | A572025Hi | +C*+C*+A*C*T*G*T*G*A*T*T*T*G*A*T*G*+ | 55118 | 19 |
| | GAGT | | A*+G*+T | | |
| 2028 | TTCCACTGTGATTTG | A572026Hi | +T*+T*+C*C*A*C*T*G*T*G*A*T*T*T*G*A*+ | 55120 | 19 |
| | ATGA | | T*+G*+A | | |
| 2029 | TGTGTGTGCCATCAT | A572027Hi | +T*+G*+T*G*T*G*T*G*C*C*A*T*C*A*T*A*+ | 55316 | 19 |
| | ACAT | | C*+A*+T | | |
| 2030 | TTGTGTAAAGTATTT | A572028Hi | +T*+T*+G*T*G*T*A*A*A*G*T*A*T*T*T*+G* | 56182 | 18 |
| | GGA | | +G*+A | | |
| 2031 | ATTGTGTAAAGTATT | A572029Hi | +A*+T*+T*G*T*G*T*A*A*A*G*T*A*T*T*T*+ | 56182 | 19 |
| | TGGA | | G*+G*+A | | |
| 2032 | ATTGTGTAAAGTATT | A572030Hi | +A*+T*+T*G*T*G*T*A*A*A*G*T*A*T*T*+T* | 56183 | 18 |
| | TGG | | +G*+G | | |
| 2033 | AATTGTGTAAAGTAT | A572031Hi | +A*+A*+T*T*G*T*G*T*A*A*A*G*T*A*T*T*+ | 56183 | 19 |
| | TTGG | | T*+G*+G | | |
| 2034 | ATTAGAACCTACTGG | A572032Hi | +A*+T*+T*A*G*A*A*C*C*T*A*C*T*G*G*+A* | 56227 | 18 |
| | ACC | | +C*+C | | |
| 2035 | AGCTTGTTCTTAGGC | A572033Hi | +A*+G*+C*T*T*G*T*T*C*T*T*A*G*G*C*A*+ | 56294 | 19 |
| | AAGT | | A*+G*+T | | |
| 2036 | GGAGCTTGTTCTTAG | A572034Hi | +G*+G*+A*G*C*T*T*G*T*T*C*T*T*A*G*G*+ | 56296 | 19 |
| | GCAA | | C*+A*+A | | |
| 2037 | CCAGGAGCTTGTTCT | A572035Hi | +C*+C*+A*G*G*A*G*C*T*T*G*T*T*C*T*T*+ | 56299 | 19 |
| | TAGG | | A*+G*+G | | |
| 2038 | ACCAGGAGCTTGTTC | A572036Hi | +A*+C*+C*A*G*G*A*G*C*T*T*G*T*T*C*T*+ | 56300 | 19 |
| | TTAG | | T*+A*+G | | |
| 2039 | GCTGTGTAGCCAAGA | A572037Hi | +G*+C*+T*G*T*G*T*A*G*C*C*A*A*G*A*+A* | 56355 | 18 |
| | AAT | | +A*+T | | |
| 2040 | TGCTGTGTAGCCAAG | A572038Hi | +T*+G*+C*T*G*T*G*T*A*G*C*C*A*A*G*A*+ | 56355 | 19 |
| | AAAT | | A*+A*+T | | |
| 2041 | TCTGCTGTGTAGCCA | A572039Hi | +T*+C*+T*G*C*T*G*T*G*T*A*G*C*C*A*A*+ | 56357 | 19 |
| | AGAA | | G*+A*+A | | |
| 2042 | ATCTGCTGTGTAGCC | A572040Hi | +A*+T*+C*T*G*C*T*G*T*G*T*A*G*C*C*A*+ | 56358 | 19 |
| | AAGA | | A*+G*+A | | |
| 2043 | TGAATCTGCTGTGTA | A572041Hi | +T*+G*+A*A*T*C*T*G*C*T*G*T*G*T*A*G*+ | 56361 | 19 |
| | GCCA | | C*+C*+A | | |
| 2044 | TGAATCTGCTGTGTA | A572042Hi | +T*+G*+A*A*T*C*T*G*C*T*G*T*G*T*A*+G* | 56362 | 18 |
| | GCC | | +C*+C | | |
| 2045 | CCATGAATCTGCTGT | A572043Hi | +C*+C*+A*T*G*A*A*T*C*T*G*C*T*G*T*G*+ | 56364 | 19 |
| | GTAG | | T*+A*+G | | |
| 2046 | CTACCATGAATCTGC | A572044Hi | +C*+T*+A*C*C*A*T*G*A*A*T*C*T*G*C*T*+ | 56367 | 19 |
| | TGTG | | G*+T*+G | | |
| 2047 | TACCATGAATCTGCT | A572045Hi | +T*+A*+C*C*A*T*G*A*A*T*C*T*G*C*T*+G* | 56367 | 18 |
| | GTG | | +T*+G | | |
| 2048 | CACTACCATGAATCT | A572046Hi | +C*+A*+C*T*A*C*C*A*T*G*A*A*T*C*T*+G* | 56370 | 18 |
| | GCT | | +C*+T | | |
| 2049 | CCACCACTACCATGA | A572047Hi | +C*+C*+A*C*C*A*C*T*A*C*C*A*T*G*A*A*+ | 56373 | 19 |
| | ATCT | | T*+C*+T | | |
| 2050 | GCAATCTTTCTCTGA | A572048Hi | +G*+C*+A*A*T*C*T*T*T*C*T*C*T*G*A*T*+ | 56469 | 19 |
| | TCAT | | C*+A*+T | | |
| 2051 | CTCTTTAGAATATTCA | A572049Hi | +C*+T*+C*T*T*T*A*G*A*A*T*A*T*T*C*A*+ | 56623 | 19 |
| | CAC | | C*+A*+C | | |
| 2052 | CCTACAGAGTCATTC | A572050Hi | +C*+C*+T*A*C*A*G*A*G*T*C*A*T*T*C*T*+ | 56637 | 19 |
| | TCTT | | C*+T*+T | | |
| 2053 | TCCTACAGAGTCATT | A572051Hi | +T*+C*+C*T*A*C*A*G*A*G*T*C*A*T*T*C*+ | 56638 | 19 |
| | CTCT | | T*+C*+T | | |
| 2054 | TTAAATCCTACAGAG | A572052Hi | +T*+T*+A*A*A*T*C*C*T*A*C*A*G*A*G*T*+ | 56643 | 19 |
| | TCAT | | C*+A*+T | | |
| 2055 | TTTAAATCCTACAGA | A572053Hi | +T*+T*+T*A*A*A*T*C*C*T*A*C*A*G*A*+G* | 56645 | 18 |
| | GTC | | +T*+C | | |
| 2056 | CTGACTTTAGGCATA | A572054Hi | +C*+T*+G*A*C*T*T*T*A*G*G*C*A*T*A*+T* | 56740 | 18 |
| | TGG | | +G*+G | | |
| 2057 | GCTGACTTTAGGCAT | A572055Hi | +G*+C*+T*G*A*C*T*T*T*A*G*G*C*A*T*A*+ | 56740 | 19 |
| | ATGG | | T*+G*+G | | |
| 2058 | CTATCCAAGCTGACT | A572056Hi | +C*+T*+A*T*C*C*A*A*G*C*T*G*A*C*T*T*+ | 56748 | 19 |
| | TTAG | | T*+A*+G | | |
| 2059 | AACTATCCAAGCTGA | A572057Hi | +A*+A*+C*T*A*T*C*C*A*A*G*C*T*G*A*C*+ | 56750 | 19 |
| | CTTT | | T*+T*+T | | |
| 2060 | AACTATCCAAGCTGA | A572058Hi | +A*+A*+C*T*A*T*C*C*A*A*G*C*T*G*A*+C* | 56751 | 18 |
| | CTT | | +T*+T | | |
| 2061 | GGATCCAAACTATCC | A572059Hi | +G*+G*+A*T*C*C*A*A*A*C*T*A*T*C*C*+A* | 56758 | 18 |
| | AAG | | +A*+G | | |
| 2062 | TCCTTGGAGGATCCA | A572060Hi | +T*+C*+C*T*T*G*G*A*G*G*A*T*C*C*A*A*+ | 56765 | 19 |
| | AACT | | A*+C*+T | | |
| 2063 | CCTTGGAGGATCCAA | A572061Hi | +C*+C*+T*T*G*G*A*G*G*A*T*C*C*A*A*+A* | 56765 | 18 |
| | ACT | | +C*+T | | |
| 2064 | TCCTTGGAGGATCCA | A572062Hi | +T*+C*+C*T*T*G*G*A*G*G*A*T*C*C*A*+A* | 56766 | 18 |
| | AAC | | +A*+C | | |
| 2065 | CTGCACATGTTTGTG | A572063Hi | +C*+T*+G*C*A*C*A*T*G*T*T*T*G*T*G*G*+ | 56790 | 19 |
| | GAAG | | A*+A*+G | | |
| 2066 | TATTAATTATCTAGCA | A572064Hi | +T*+A*+T*T*A*A*T*T*A*T*C*T*A*G*C*A*+ | 56813 | 19 |
| | CTG | | C*+T*+G | | |
| 2067 | GTACTAAGGTGATTT | A572065Hi | +G*+T*+A*C*T*A*A*G*G*T*G*A*T*T*T*A*+ | 56907 | 19 |
| | ATGT | | T*+G*+T | | |
| 2068 | GTACTAAGGTGATTT | A572066Hi | +G*+T*+A*C*T*A*A*G*G*T*G*A*T*T*T*+A* | 56908 | 18 |
| | ATG | | +T*+G | | |
| 2069 | TGTACTAAGGTGATT | A572067Hi | +T*+G*+T*A*C*T*A*A*G*G*T*G*A*T*T*T*+ | 56908 | 19 |
| | TATG | | A*+T*+G | | |
| 2070 | CTTTGTACTAAGGTG | A572068Hi | +C*+T*+T*T*G*T*A*C*T*A*A*G*G*T*G*A*+ | 56911 | 19 |
| | ATTT | | T*+T*+T | | |
| 2071 | CTTTGTACTAAGGTG | A572069Hi | +C*+T*+T*T*G*T*A*C*T*A*A*G*G*T*G*+A* | 56912 | 18 |
| | ATT | | +T*+T | | |
| 2072 | TCACTCCTTACTTTGT | A572070Hi | +T*+C*+A*C*T*C*C*T*T*A*C*T*T*T*G*T*+ | 56921 | 19 |
| | ACT | | A*+C*+T | | |
| 2073 | CACTCCTTACTTTGTA | A572071Hi | +C*+A*+C*T*C*C*T*T*A*C*T*T*T*G*T*+A* | 56921 | 18 |
| | CT | | +C*+T | | |
| 2074 | CCTGAAGCCTAGCAC | A572072Hi | +C*+C*+T*G*A*A*G*C*C*T*A*G*C*A*C*A*+ | 57305 | 19 |
| | AACA | | A*+C*+A | | |
| 2075 | CCTGAAGCCTAGCAC | A572073Hi | +C*+C*+T*G*A*A*G*C*C*T*A*G*C*A*C*+A* | 57306 | 18 |
| | AAC | | +A*+C | | |
| 2076 | TCCTGAAGCCTAGCA | A572074Hi | +T*+C*+C*T*G*A*A*G*C*C*T*A*G*C*A*C*+ | 57306 | 19 |
| | CAAC | | A*+A*+C | | |
| 2077 | ACTTGTATTCCTGAA | A572075Hi | +A*+C*+T*T*G*T*A*T*T*C*C*T*G*A*A*G*+ | 57314 | 19 |
| | GCCT | | C*+C*+T | | |
| 2078 | TCTTGCTAGAATGCA | A572076Hi | +T*+C*+T*T*G*C*T*A*G*A*A*T*G*C*A*A*+ | 57354 | 19 |
| | AAGG | | A*+G*+G | | |
| 2079 | ATGTAGCACTGAGCA | A572077Hi | +A*+T*+G*T*A*G*C*A*C*T*G*A*G*C*A*A*+ | 57390 | 19 |
| | AACA | | A*+C*+A | | |
| 2080 | TCATTTAATGTAGCA | A572078Hi | +T*+C*+A*T*T*T*A*A*T*G*T*A*G*C*A*C*+ | 57397 | 19 |
| | CTGA | | T*+G*+A | | |
| 2081 | TTCATTTAATGTAGC | A572079Hi | +T*+T*+C*A*T*T*T*A*A*T*G*T*A*G*C*A*+ | 57398 | 19 |
| | ACTG | | C*+T*+G | | |
| 2082 | CTACTAACCTCTCAG | A572080Hi | +C*+T*+A*C*T*A*A*C*C*T*C*T*C*A*G*+G* | 57451 | 18 |
| | GCC | | +C*+C | | |
| 2083 | GCTACTAACCTCTCA | A572081Hi | +G*+C*+T*A*C*T*A*A*C*C*T*C*T*C*A*G*+ | 57451 | 19 |
| | GGCC | | G*+C*+C | | |
| 2084 | CCTTGCTACTAACCT | A572082Hi | +C*+C*+T*T*G*C*T*A*C*T*A*A*C*C*T*C*+ | 57455 | 19 |
| | CTCA | | T*+C*+A | | |
| 2085 | GTCCTTGCTACTAAC | A572083Hi | +G*+T*+C*C*T*T*G*C*T*A*C*T*A*A*C*+C* | 57458 | 18 |
| | CTC | | +T*+C | | |
| 2086 | GTTCATTCTTGAAGA | A572084Hi | +G*+T*+T*C*A*T*T*C*T*T*G*A*A*G*A*A*+ | 57499 | 19 |
| | ATTG | | T*+T*+G | | |
| 2087 | TGTTCATTCTTGAAG | A572085Hi | +T*+G*+T*T*C*A*T*T*C*T*T*G*A*A*G*A*+ | 57500 | 19 |
| | AATT | | A*+T*+T | | |
| 2088 | TCTTCCAAACACAGC | A572086Hi | +T*+C*+T*T*C*C*A*A*A*C*A*C*A*G*C*A*+ | 57598 | 19 |
| | ATGT | | T*+G*+T | | |
| 2089 | TCAAAGCACTACAGA | A572087Hi | +T*+C*+A*A*A*G*C*A*C*T*A*C*A*G*A*G*+ | 57673 | 19 |
| | GCTC | | C*+T*+C | | |
| 2090 | CCACCAGTCTCCAAG | A572088Hi | +C*+C*+A*C*C*A*G*T*C*T*C*C*A*A*G*+T* | 57711 | 18 |
| | TAC | | +A*+C | | |
| 2091 | ATGTGGTTGGAAGGC | A572089Hi | +A*+T*+G*T*G*G*T*T*G*G*A*A*G*G*C*A*+ | 57738 | 19 |
| | ACCA | | C*+C*+A | | |
| 2092 | ATGTGGTTGGAAGGC | A572090Hi | +A*+T*+G*T*G*G*T*T*G*G*A*A*G*G*C*+A* | 57739 | 18 |
| | ACC | | +C*+C | | |
| 2093 | CAAATCTGCTGGCTT | A572091Hi | +C*+A*+A*A*T*C*T*G*C*T*G*G*C*T*T*C*+ | 57917 | 19 |
| | CAAA | | A*+A*+A | | |
| 2094 | TATGCCATAGCAATT | A572092Hi | +T*+A*+T*G*C*C*A*T*A*G*C*A*A*T*T*+A* | 58021 | 18 |
| | ATT | | +T*+T | | |
| 2095 | TGTGCTAAATTCTGT | A572093Hi | +T*+G*+T*G*C*T*A*A*A*T*T*C*T*G*T*A*+ | 58072 | 19 |
| | ACAC | | C*+A*+C | | |
| 2096 | ATATCCATCAGTTAC | A572094Hi | +A*+T*+A*T*C*C*A*T*C*A*G*T*T*A*C*T*+ | 58087 | 19 |
| | TGTG | | G*+T*+G | | |
| 2097 | TAATATCCATCAGTT | A572095Hi | +T*+A*+A*T*A*T*C*C*A*T*C*A*G*T*T*A*+ | 58089 | 19 |
| | ACTG | | C*+T*+G | | |
| 2098 | AATAATATCCATCAG | A572096Hi | +A*+A*+T*A*A*T*A*T*C*C*A*T*C*A*G*T*+ | 58091 | 19 |
| | TTAC | | T*+A*+C | | |
| 2099 | GTAATAGAAATAATA | A572097Hi | +G*+T*+A*A*T*A*G*A*A*A*T*A*A*T*A*T*+ | 58099 | 19 |
| | TCCA | | C*+C*+A | | |
| 2100 | GGTAATAGAAATAAT | A572098Hi | +G*+G*+T*A*A*T*A*G*A*A*A*T*A*A*T*A*+ | 58100 | 19 |
| | ATCC | | T*+C*+C | | |
| 2101 | GGAGCTATCAACTAG | A572099Hi | +G*+G*+A*G*C*T*A*T*C*A*A*C*T*A*G*A*+ | 58130 | 19 |
| | AAGA | | A*+G*+A | | |
| 2102 | GCAAGGAGCTATCAA | A572100Hi | +G*+C*+A*A*G*G*A*G*C*T*A*T*C*A*A*C*+ | 58134 | 19 |
| | CTAG | | T*+A*+G | | |
| 2103 | TTCCTCAAAGATATA | A572101Hi | +T*+T*+C*C*T*C*A*A*A*G*A*T*A*T*A*C*+ | 58334 | 19 |
| | CCTT | | C*+T*+T | | |
| 2104 | TGTGACATAGAATAA | A572102Hi | +T*+G*+T*G*A*C*A*T*A*G*A*A*T*A*A*+C* | 58454 | 18 |
| | CTA | | +T*+A | | |
| 2105 | AACCAGTACCTTAAC | A572103Hi | +A*+A*+C*C*A*G*T*A*C*C*T*T*A*A*C*A*+ | 58497 | 19 |
| | ACAG | | C*+A*+G | | |
| 2106 | AACCAGTACCTTAAC | A572104Hi | +A*+A*+C*C*A*G*T*A*C*C*T*T*A*A*C*+A* | 58498 | 18 |
| | ACA | | +C*+A | | |
| 2107 | AAACCAGTACCTTAA | A572105Hi | +A*+A*+A*C*C*A*G*T*A*C*C*T*T*A*A*+C* | 58499 | 18 |
| | CAC | | +A*+C | | |
| 2108 | TGTTTACATCCAGAC | A572106Hi | +T*+G*+T*T*T*A*C*A*T*C*C*A*G*A*C*T*+ | 58578 | 19 |
| | TCCA | | C*+C*+A | | |
| 2109 | TCTGTTTACATCCAG | A572107Hi | +T*+C*+T*G*T*T*T*A*C*A*T*C*C*A*G*A*+ | 58580 | 19 |
| | ACTC | | C*+T*+C | | |
| 2110 | TCTCTGTTTACATCCA | A572108Hi | +T*+C*+T*C*T*G*T*T*T*A*C*A*T*C*C*A*+ | 58582 | 19 |
| | GAC | | G*+A*+C | | |
| 2111 | ATCTCTGTTTACATCC | A572109Hi | +A*+T*+C*T*C*T*G*T*T*T*A*C*A*T*C*C*+ | 58583 | 19 |
| | AGA | | A*+G*+A | | |
| 2112 | AAGATCTCTGTTTAC | A572110Hi | +A*+A*+G*A*T*C*T*C*T*G*T*T*T*A*C*A*+ | 58586 | 19 |
| | ATCC | | T*+C*+C | | |
| 2113 | AAGATCTCTGTTTAC | A572111Hi | +A*+A*+G*A*T*C*T*C*T*G*T*T*T*A*C*+A* | 58587 | 18 |
| | ATC | | +T*+C | | |
| 2114 | TCAAGATCTCTGTTT | A572112Hi | +T*+C*+A*A*G*A*T*C*T*C*T*G*T*T*T*A*+ | 58588 | 19 |
| | ACAT | | C*+A*+T | | |
| 2115 | GGCTTGGCATCAAGA | A572113Hi | +G*+G*+C*T*T*G*G*C*A*T*C*A*A*G*A*T*+ | 58597 | 19 |
| | TCTC | | C*+T*+C | | |
| 2116 | TAGCATGAATAATCT | A572114Hi | +T*+A*+G*C*A*T*G*A*A*T*A*A*T*C*T*G*+ | 58671 | 19 |
| | GCCA | | C*+C*+A | | |
| 2117 | GAATAGCATGAATAA | A572115Hi | +G*+A*+A*T*A*G*C*A*T*G*A*A*T*A*A*T*+ | 58674 | 19 |
| | TCTG | | C*+T*+G | | |
| 2118 | GATCCTTGAATAGAT | A572116Hi | +G*+A*+T*C*C*T*T*G*A*A*T*A*G*A*T*+T* | 58726 | 18 |
| | TGG | | +G*+G | | |
| 2119 | TACATCTCCAATTTG | A572117Hi | +T*+A*+C*A*T*C*T*C*C*A*A*T*T*T*G*G*+ | 58770 | 19 |
| | GAAT | | A*+A*+T | | |
| 2120 | TGGGACTGCTCTCTT | A572118H | +T*+G*+G*G*A*C*T*G*C*T*C*T*C*T*T*T*+ | 58885 | 19 |
| | TCTC | | C*+T*+C | | |
| 2121 | CAATTTGGGACTGCT | A572119H | +C*+A*+A*T*T*T*G*G*G*A*C*T*G*C*T*C*+ | 58890 | 19 |
| | CTCT | | T*+C*+T | | |
| 2122 | CAATTTGGGACTGCT | A572120H | +C*+A*+A*T*T*T*G*G*G*A*C*T*G*C*T*+C* | 58891 | 18 |
| | CTC | | +T*+C | | |
| 2123 | CCAATTTGGGACTGC | A572121H | +C*+C*+A*A*T*T*T*G*G*G*A*C*T*G*C*T*+ | 58891 | 19 |
| | TCTC | | C*+T*+C | | |
| 2124 | ACCAATTTGGGACTG | A572122H | +A*+C*+C*A*A*T*T*T*G*G*G*A*C*T*G*C*+ | 58892 | 19 |
| | CTCT | | T*+C*+T | | |
| 2125 | ACCAATTTGGGACTG | A572123H | +A*+C*+C*A*A*T*T*T*G*G*G*A*C*T*G*+C* | 58893 | 18 |
| | CTC | | +T*+C | | |
| 2126 | CACCAATTTGGGACT | A572124H | +C*+A*+C*C*A*A*T*T*T*G*G*G*A*C*T*G*+ | 58893 | 19 |
| | GCTC | | C*+T*+C | | |
| 2127 | TCCACCAATTTGGGA | A572125H | +T*+C*+C*A*C*C*A*A*T*T*T*G*G*G*A*C*+ | 58895 | 19 |
| | CTGC | | T*+G*+C | | |
| 2128 | GTTCCACCAATTTGG | A572126H | +G*+T*+T*C*C*A*C*C*A*A*T*T*T*G*G*G*+ | 58897 | 19 |
| | GACT | | A*+C*+T | | |
| 2129 | AGTTCCACCAATTTG | A572127H | +A*+G*+T*T*C*C*A*C*C*A*A*T*T*T*G*G*+ | 58898 | 19 |
| | GGAC | | G*+A*+C | | |
| 2130 | ACGAGATCCACTATT | A572128H | +A*+C*+G*A*G*A*T*C*C*A*C*T*A*T*+T*+C | 58922 | 17 |
| | CA | | *+A | | |
| 2131 | CACGAGATCCACTAT | A572129H | +C*+A*+C*G*A*G*A*T*C*C*A*C*T*A*T*+T* | 58922 | 18 |
| | TCA | | +C*+A | | |
| 2132 | TCACGAGATCCACTA | A572130H | +T*+C*+A*C*G*A*G*A*T*C*C*A*C*T*A*T*+ | 58922 | 19 |
| | TTCA | | T*+C*+A | | |
| 2133 | TCACGAGATCCACTA | A572131H | +T*+C*+A*C*G*A*G*A*T*C*C*A*C*T*A*+T* | 58923 | 18 |
| | TTC | | +T*+C | | |
| 2134 | TTCACGAGATCCACT | A572132H | +T*+T*+C*A*C*G*A*G*A*T*C*C*A*C*T*A*+ | 58923 | 19 |
| | ATTC | | T*+T*+C | | |
| 2135 | GTTCACGAGATCCAC | A572133H | +G*+T*+T*C*A*C*G*A*G*A*T*C*C*A*C*T*+ | 58924 | 19 |
| | TATT | | A*+T*+T | | |
| 2136 | GTTCACGAGATCCAC | A572134H | +G*+T*+T*C*A*C*G*A*G*A*T*C*C*A*C*+T* | 58925 | 18 |
| | TAT | | +A*+T | | |
| 2137 | TGTTCACGAGATCCA | A572135H | +T*+G*+T*T*C*A*C*G*A*G*A*T*C*C*A*C*+ | 58925 | 19 |
| | CTAT | | T*+A*+T | | |
| 2138 | GTTCACGAGATCCAC | A572136H | +G*+T*+T*C*A*C*G*A*G*A*T*C*C*A*+C*+T | 58926 | 17 |
| | TA | | *+A | | |
| 2139 | TGTTCACGAGATCCA | A572137H | +T*+G*+T*T*C*A*C*G*A*G*A*T*C*C*A*+C* | 58926 | 18 |
| | CTA | | +T*+A | | |
| 2140 | TTGTTCACGAGATCC | A572138H | +T*+T*+G*T*T*C*A*C*G*A*G*A*T*C*C*A*+ | 58926 | 19 |
| | ACTA | | C*+T*+A | | |
| 2141 | TTGTTCACGAGATCC | A572139H | +T*+T*+G*T*T*C*A*C*G*A*G*A*T*C*C*+A* | 58927 | 18 |
| | ACT | | +C*+T | | |
| 2142 | CTTGTTCACGAGATC | A572140H | +C*+T*+T*G*T*T*C*A*C*G*A*G*A*T*C*C*+ | 58927 | 19 |
| | CACT | | A*+C*+T | | |
| 2143 | TTGTTCACGAGATCC | A572141H | +T*+T*+G*T*T*C*A*C*G*A*G*A*T*C*+C*+A | 58928 | 17 |
| | AC | | *+C | | |
| 2144 | CTTGTTCACGAGATC | A572142H | +C*+T*+T*G*T*T*C*A*C*G*A*G*A*T*C*+C* | 58928 | 18 |
| | CAC | | +A*+C | | |
| 2145 | TCTTGTTCACGAGAT | A572143H | +T*+C*+T*T*G*T*T*C*A*C*G*A*G*A*T*C*+ | 58928 | 19 |
| | CCAC | | C*+A*+C | | |
| 2146 | ATCTTGTTCACGAGA | A572144H | +A*+T*+C*T*T*G*T*T*C*A*C*G*A*G*A*T*+ | 58929 | 19 |
| | TCCA | | C*+C*+A | | |
| 2147 | ATCTTGTTCACGAGA | A572145H | +A*+T*+C*T*T*G*T*T*C*A*C*G*A*G*A*+T* | 58930 | 18 |
| | TCC | | +C*+C | | |
| 2148 | CATCTTGTTCACGAG | A572146H | +C*+A*+T*C*T*T*G*T*T*C*A*C*G*A*G*A*+ | 58930 | 19 |
| | ATCC | | T*+C*+C | | |
| 2149 | CATCTTGTTCACGAG | A572147H | +C*+A*+T*C*T*T*G*T*T*C*A*C*G*A*G*+A* | 58931 | 18 |
| | ATC | | +T*+C | | |
| 2150 | ACATCTTGTTCACGA | A572148H | +A*+C*+A*T*C*T*T*G*T*T*C*A*C*G*A*G*+ | 58931 | 19 |
| | GATC | | A*+T*+C | | |
| 2151 | ACATCTTGTTCACGA | A572149H | +A*+C*+A*T*C*T*T*G*T*T*C*A*C*G*A*+G* | 58932 | 18 |
| | GAT | | +A*+T | | |
| 2152 | TACATCTTGTTCACG | A572150H | +T*+A*+C*A*T*C*T*T*G*T*T*C*A*C*G*A*+ | 58932 | 19 |
| | AGAT | | G*+A*+T | | |
| 2153 | GTACATCTTGTTCAC | A572151H | +G*+T*+A*C*A*T*C*T*T*G*T*T*C*A*C*G*+ | 58933 | 19 |
| | GAGA | | A*+G*+A | | |
| 2154 | GTACATCTTGTTCAC | A572152H | +G*+T*+A*C*A*T*C*T*T*G*T*T*C*A*C*+G* | 58934 | 18 |
| | GAG | | +A*+G | | |
| 2155 | CGTACATCTTGTTCA | A572153H | +C*+G*+T*A*C*A*T*C*T*T*G*T*T*C*A*C*+ | 58934 | 19 |
| | CGAG | | G*+A*+G | | |
| 2156 | CGTACATCTTGTTCA | A572154H | +C*+G*+T*A*C*A*T*C*T*T*G*T*T*C*A*+C* | 58935 | 18 |
| | CGA | | +G*+A | | |
| 2157 | TCGTACATCTTGTTC | A572155H | +T*+C*+G*T*A*C*A*T*C*T*T*G*T*T*C*A*+ | 58935 | 19 |
| | ACGA | | C*+G*+A | | |
| 2158 | TTCGTACATCTTGTTC | A572156H | +T*+T*+C*G*T*A*C*A*T*C*T*T*G*T*T*C*+ | 58936 | 19 |
| | ACG | | A*+C*+G | | |
| 2159 | CTTTTCGTACATCTTG | A572157H | +C*+T*+T*T*T*C*G*T*A*C*A*T*C*T*T*G*+ | 58939 | 19 |
| | TTC | | T*+T*+C | | |
| 2160 | GCTGATGATCTGGCT | A572158H | +G*+C*+T*G*A*T*G*A*T*C*T*G*G*C*T*+G* | 58984 | 18 |
| | GTC | | +T*+C | | |
| 2161 | AGCTGATGATCTGGC | A572159H | +A*+G*+C*T*G*A*T*G*A*T*C*T*G*G*C*T*+ | 58984 | 19 |
| | TGTC | | G*+T*+C | | |
| 2162 | AGCTGATGATCTGGC | A572160H | +A*+G*+C*T*G*A*T*G*A*T*C*T*G*G*C*+T* | 58985 | 18 |
| | TGT | | +G*+T | | |
| 2163 | AAGCTGATGATCTGG | A572161H | +A*+A*+G*C*T*G*A*T*G*A*T*C*T*G*G*C*+ | 58985 | 19 |
| | CTGT | | T*+G*+T | | |
| 2164 | CAAGCTGATGATCTG | A572162H | +C*+A*+A*G*C*T*G*A*T*G*A*T*C*T*G*G*+ | 58986 | 19 |
| | GCTG | | C*+T*+G | | |
| 2165 | GCAAGCTGATGATCT | A572163H | +G*+C*+A*A*G*C*T*G*A*T*G*A*T*C*T*G*+ | 58987 | 19 |
| | GGCT | | G*+C*+T | | |
| 2166 | AGCAAGCTGATGATC | A572164H | +A*+G*+C*A*A*G*C*T*G*A*T*G*A*T*C*T*+ | 58988 | 19 |
| | TGGC | | G*+G*+C | | |
| 2167 | TAAGAGCAAGCTGAT | A572165H | +T*+A*+A*G*A*G*C*A*A*G*C*T*G*A*T*G*+ | 58992 | 19 |
| | GATC | | A*+T*+C | | |
| 2168 | TTAAGAGCAAGCTGA | A572166H | +T*+T*+A*A*G*A*G*C*A*A*G*C*T*G*A*T*+ | 58993 | 19 |
| | TGAT | | G*+A*+T | | |
| 2169 | CCTTAAGAGCAAGCT | A572167H | +C*+C*+T*T*A*A*G*A*G*C*A*A*G*C*T*G*+ | 58995 | 19 |
| | GATG | | A*+T*+G | | |
| 2170 | CCTTAAGAGCAAGCT | A572168H | +C*+C*+T*T*A*A*G*A*G*C*A*A*G*C*T*+G* | 58996 | 18 |
| | GAT | | +A*+T | | |
| 2171 | TCCTTAAGAGCAAGC | A572169H | +T*+C*+C*T*T*A*A*G*A*G*C*A*A*G*C*T*+ | 58996 | 19 |
| | TGAT | | G*+A*+T | | |
| 2172 | CCTCCTTAAGAGCAA | A572170H | +C*+C*+T*C*C*T*T*A*A*G*A*G*C*A*A*G*+ | 58998 | 19 |
| | GCTG | | C*+T*+G | | |
| 2173 | GGCCAGCCTCCTTAA | A572171H | +G*+G*+C*C*A*G*C*C*T*C*C*T*T*A*A*G*+ | 59004 | 19 |
| | GAGC | | A*+G*+C | | |
| 2174 | GGGCCAGCCTCCTTA | A572172H | +G*+G*+G*C*C*A*G*C*C*T*C*C*T*T*A*A*+ | 59005 | 19 |
| | AGAG | | G*+A*+G | | |
| 2175 | CAGGGCCAGCCTCCT | A572173H | +C*+A*+G*G*G*C*C*A*G*C*C*T*C*C*T*T*+ | 59007 | 19 |
| | TAAG | | A*+A*+G | | |
| 2176 | CAGGGCCAGCCTCCT | A572174H | +C*+A*+G*G*G*C*C*A*G*C*C*T*C*C*T*+T* | 59008 | 18 |
| | TAA | | +A*+A | | |
| 2177 | CCAGGGCCAGCCTCC | A572175H | +C*+C*+A*G*G*G*C*C*A*G*C*C*T*C*C*T*+ | 59008 | 19 |
| | TTAA | | T*+A*+A | | |
| 2178 | CAAATGCTATTGTTG | A572176H | +C*+A*+A*A*T*G*C*T*A*T*T*G*T*T*G*G*+ | 59030 | 19 |
| | GCCA | | C*+C*+A | | |
| 2179 | GCAAATGCTATTGTT | A572177H | +G*+C*+A*A*A*T*G*C*T*A*T*T*G*T*T*G*+ | 59031 | 19 |
| | GGCC | | G*+C*+C | | |
| 2180 | AGGCAAATGCTATTG | A572178H | +A*+G*+G*C*A*A*A*T*G*C*T*A*T*T*G*T*+ | 59033 | 19 |
| | TTGG | | T*+G*+G | | |
| 2181 | TCAACTTTTCCTATAC | A572179H | +T*+C*+A*A*C*T*T*T*T*C*C*T*A*T*A*C*+ | 59060 | 19 |
| | AAA | | A*+A*+A | | |
| 2182 | GTTCAACTTTTCCTAT | A572180H | +G*+T*+T*C*A*A*C*T*T*T*T*C*C*T*A*T*+ | 59062 | 19 |
| | ACA | | A*+C*+A | | |
| 2183 | GGTTCAACTTTTCCT | A572181H | +G*+G*+T*T*C*A*A*C*T*T*T*T*C*C*T*A*+ | 59063 | 19 |
| | ATAC | | T*+A*+C | | |
| 2184 | AAGGTTCAACTTTTC | A572182H | +A*+A*+G*G*T*T*C*A*A*C*T*T*T*T*C*C*+ | 59065 | 19 |
| | CTAT | | T*+A*+T | | |
| 2185 | GAAGGTTCAACTTTT | A572183H | +G*+A*+A*G*G*T*T*C*A*A*C*T*T*T*T*C*+ | 59066 | 19 |
| | CCTA | | C*+T*+A | | |
| 2186 | AAGAAGGTTCAACTT | A572184H | +A*+A*+G*A*A*G*G*T*T*C*A*A*C*T*T*T*+ | 59068 | 19 |
| | TTCC | | T*+C*+C | | |
| 2187 | CCAAGAAGGTTCAAC | A572185H | +C*+C*+A*A*G*A*A*G*G*T*T*C*A*A*C*T*+ | 59070 | 19 |
| | TTTT | | T*+T*+T | | |
| 2188 | GCCAAGAAGGTTCAA | A572186H | +G*+C*+C*A*A*G*A*A*G*G*T*T*C*A*A*C*+ | 59071 | 19 |
| | CTTT | | T*+T*+T | | |
| 2189 | GGACCAAGCCAAGAA | A572187H | +G*+G*+A*C*C*A*A*G*C*C*A*A*G*A*A*G* | 59078 | 19 |
| | GGTT | | +G*+T*+T | | |
| 2190 | AGGACCAAGCCAAGA | A572188H | +A*+G*+G*A*C*C*A*A*G*C*C*A*A*G*A*A* | 59079 | 19 |
| | AGGT | | +G*+G*+T | | |
| 2191 | TGGAAATAAAGGACC | A572189H | +T*+G*+G*A*A*A*T*A*A*A*G*G*A*C*C*A*+ | 59088 | 19 |
| | AAGC | | A*+G*+C | | |
| 2192 | AAGTCTTATCTGGAA | A572190H | +A*+A*+G*T*C*T*T*A*T*C*T*G*G*A*A*A*+ | 59098 | 19 |
| | ATAA | | T*+A*+A | | |
| 2193 | GAAGTCTTATCTGGA | A572191H | +G*+A*+A*G*T*C*T*T*A*T*C*T*G*G*A*A*+ | 59099 | 19 |
| | AATA | | A*+T*+A | | |
| 2194 | TTAGAAGTCTTATCT | A572192H | +T*+T*+A*G*A*A*G*T*C*T*T*A*T*C*T*G*+ | 59102 | 19 |
| | GGAA | | G*+A*+A | | |
| 2195 | TCCTTAAATTAGAAG | A572193H | +T*+C*+C*T*T*A*A*A*T*T*A*G*A*A*G*T*+ | 59110 | 19 |
| | TCTT | | C*+T*+T | | |
| 2196 | TTTCCTTAAATTAGAA | A572194H | +T*+T*+T*C*C*T*T*A*A*A*T*T*A*G*A*A*+ | 59112 | 19 |
| | GTC | | G*+T*+C | | |
| 2197 | AAATATTCTCCTTGTT | A572195Hi | +A*+A*+A*T*A*T*T*C*T*C*C*T*T*G*T*T*+ | 59141 | 19 |
| | ACT | | A*+C*+T | | |
| 2198 | CTATCTCATAAAGAA | A572196Hi | +C*+T*+A*T*C*T*C*A*T*A*A*A*G*A*A*C*+ | 59307 | 19 |
| | CTTA | | T*+T*+A | | |
| 2199 | GCTATCTCATAAAGA | A572197Hi | +G*+C*+T*A*T*C*T*C*A*T*A*A*A*G*A*A*+ | 59308 | 19 |
| | ACTT | | C*+T*+T | | |
| 2200 | TTTGCTATCTCATAAA | A572198Hi | +T*+T*+T*G*C*T*A*T*C*T*C*A*T*A*A*A*+ | 59311 | 19 |
| | GAA | | G*+A*+A | | |
| 2201 | ATAGGTACATGTAAG | A572199Hi | +A*+T*+A*G*G*T*A*C*A*T*G*T*A*A*G*C*+ | 59519 | 19 |
| | CATA | | A*+T*+A | | |
| 2202 | GCATAGGTACATGTA | A572200Hi | +G*+C*+A*T*A*G*G*T*A*C*A*T*G*T*A*+A* | 59522 | 18 |
| | AGC | | +G*+C | | |
| 2203 | TGCATAGGTACATGT | A572201Hi | +T*+G*+C*A*T*A*G*G*T*A*C*A*T*G*T*+A* | 59523 | 18 |
| | AAG | | +A*+G | | |
| 2204 | GAATGCATAGGTACA | A572202Hi | +G*+A*+A*T*G*C*A*T*A*G*G*T*A*C*A*+T* | 59526 | 18 |
| | TGT | | +G*+T | | |
| 2205 | ATTTGAATGCATAGG | A572203Hi | +A*+T*+T*T*G*A*A*T*G*C*A*T*A*G*G*T*+ | 59529 | 19 |
| | TACA | | A*+C*+A | | |
| 2206 | CATTTGAATGCATAG | A572204Hi | +C*+A*+T*T*T*G*A*A*T*G*C*A*T*A*G*G*+ | 59530 | 19 |
| | GTAC | | T*+A*+C | | |
| 2207 | GCATTATCAATTGAA | A572205Hi | +G*+C*+A*T*T*A*T*C*A*A*T*T*G*A*A*A*+ | 59604 | 19 |
| | ATAT | | T*+A*+T | | |
| 2208 | TGCATTATCAATTGA | A572206Hi | +T*+G*+C*A*T*T*A*T*C*A*A*T*T*G*A*A*+ | 59605 | 19 |
| | AATA | | A*+T*+A | | |
| 2209 | ACTTACCATCAATAC | A572207Hi | +A*+C*+T*T*A*C*C*A*T*C*A*A*T*A*C*A*+ | 59666 | 19 |
| | ACAT | | C*+A*+T | | |
| 2210 | ACATTTAGTGTGAAC | A572208Hi | +A*+C*+A*T*T*T*A*G*T*G*T*G*A*A*C*A*+ | 59725 | 19 |
| | AAAT | | A*+A*+T | | |
| 2211 | GTAGACTGGTAGACT | A572209Hi | +G*+T*+A*G*A*C*T*G*G*T*A*G*A*C*T*G*+ | 59913 | 19 |
| | GACC | | A*+C*+C | | |
| 2212 | AAAGTAGACTGGTAG | A572210Hi | +A*+A*+A*G*T*A*G*A*C*T*G*G*T*A*G*A*+ | 59916 | 19 |
| | ACTG | | C*+T*+G | | |
| 2213 | AGGCACCAAATTGCA | A572211Hi | +A*+G*+G*C*A*C*C*A*A*A*T*T*G*C*A*T*+ | 59985 | 19 |
| | TGGA | | G*+G*+A | | |
| 2214 | CTTGATGTATAAAGG | A572212Hi | +C*+T*+T*G*A*T*G*T*A*T*A*A*A*G*G*C*+ | 59997 | 19 |
| | CACC | | A*+C*+C | | |
| 2215 | CCTTGATGTATAAAG | A572213Hi | +C*+C*+T*T*G*A*T*G*T*A*T*A*A*A*G*G*+ | 59998 | 19 |
| | GCAC | | C*+A*+C | | |
| 2216 | CTTGATGTATAAAGG | A572214Hi | +C*+T*+T*G*A*T*G*T*A*T*A*A*A*G*G*+C* | 59998 | 18 |
| | CAC | | +A*+C | | |
| 2217 | ACCTTGATGTATAAA | A572215Hi | +A*+C*+C*T*T*G*A*T*G*T*A*T*A*A*A*G*+ | 59999 | 19 |
| | GGCA | | G*+C*+A | | |
| 2218 | ACCTTGATGTATAAA | A572216Hi | +A*+C*+C*T*T*G*A*T*G*T*A*T*A*A*A*+G* | 60000 | 18 |
| | GGC | | +G*+C | | |
| 2219 | AACCTTGATGTATAA | A572217Hi | +A*+A*+C*C*T*T*G*A*T*G*T*A*T*A*A*+A* | 60001 | 18 |
| | AGG | | +G*+G | | |
| 2220 | TGGAAACCTTGATGT | A572218Hi | +T*+G*+G*A*A*A*C*C*T*T*G*A*T*G*T*A*+ | 60004 | 19 |
| | ATAA | | T*+A*+A | | |
| 2221 | TGGAAACCTTGATGT | A572219Hi | +T*+G*+G*A*A*A*C*C*T*T*G*A*T*G*T*+A* | 60005 | 18 |
| | ATA | | +T*+A | | |
| 2222 | GTTGGAAACCTTGAT | A572220Hi | +G*+T*+T*G*G*A*A*A*C*C*T*T*G*A*T*G*+ | 60006 | 19 |
| | GTAT | | T*+A*+T | | |
| 2223 | GTGTTTGAGAAACCG | A572221Hi | +G*+T*+G*T*T*T*G*A*G*A*A*A*C*C*G*A*+ | 60064 | 19 |
| | AGGA | | G*+G*+A | | |
| 2224 | AAGTGTTTGAGAAAC | A572222Hi | +A*+A*+G*T*G*T*T*T*G*A*G*A*A*A*C*C*+ | 60066 | 19 |
| | CGAG | | G*+A*+G | | |
| 2225 | GGTGAAGAGAGAATT | A572223Hi | +G*+G*+T*G*A*A*G*A*G*A*G*A*A*T*T*T*+ | 60137 | 19 |
| | TAGA | | A*+G*+A | | |
| 2226 | AGCCAGGTGAAGAGA | A572224Hi | +A*+G*+C*C*A*G*G*T*G*A*A*G*A*G*A*G* | 60142 | 19 |
| | GAAT | | +A*+A*+T | | |
| 2227 | ATGTGGTGAATGGTA | A572225Hi | +A*+T*+G*T*G*G*T*G*A*A*T*G*G*T*A*+G* | 60245 | 18 |
| | GTA | | +T*+A | | |
| 2228 | ATAATGTGGTGAATG | A572226Hi | +A*+T*+A*A*T*G*T*G*G*T*G*A*A*T*G*G*+ | 60247 | 19 |
| | GTAG | | T*+A*+G | | |
| 2229 | ATATAATGTGGTGAA | A572227Hi | +A*+T*+A*T*A*A*T*G*T*G*G*T*G*A*A*T*+ | 60249 | 19 |
| | TGGT | | G*+G*+T | | |
| 2230 | GACTTCATGGTACTA | A572228Hi | +G*+A*+C*T*T*C*A*T*G*G*T*A*C*T*A*A*+ | 60307 | 19 |
| | ACAA | | C*+A*+A | | |
| 2231 | ATTGACTTCATGGTA | A572229Hi | +A*+T*+T*G*A*C*T*T*C*A*T*G*G*T*A*C*+ | 60310 | 19 |
| | CTAA | | T*+A*+A | | |
| 2232 | CATTGACTTCATGGT | A572230Hi | +C*+A*+T*T*G*A*C*T*T*C*A*T*G*G*T*A*+ | 60311 | 19 |
| | ACTA | | C*+T*+A | | |
| 2233 | GGTAAGGTTACAACC | A572231Hi | +G*+G*+T*A*A*G*G*T*T*A*C*A*A*C*C*+A* | 60341 | 18 |
| | ATG | | +T*+G | | |
| 2234 | GTATCAATGGTAAGG | A572232Hi | +G*+T*+A*T*C*A*A*T*G*G*T*A*A*G*G*+T* | 60349 | 18 |
| | TTA | | +T*+A | | |
| 2235 | ATAGATTCCTAACAG | A572233Hi | +A*+T*+A*G*A*T*T*C*C*T*A*A*C*A*G*+T* | 60575 | 18 |
| | TAC | | +A*+C | | |
| 2236 | TCATAGATTCCTAAC | A572234Hi | +T*+C*+A*T*A*G*A*T*T*C*C*T*A*A*C*A*+ | 60576 | 19 |
| | AGTA | | G*+T*+A | | |
| 2237 | TGTGCACATTGAGAA | A572235Hi | +T*+G*+T*G*C*A*C*A*T*T*G*A*G*A*A*T*+ | 60599 | 19 |
| | TGTC | | G*+T*+C | | |
| 2238 | TTGTGCACATTGAGA | A572236Hi | +T*+T*+G*T*G*C*A*C*A*T*T*G*A*G*A*A*+ | 60600 | 19 |
| | ATGT | | T*+G*+T | | |
| 2239 | TTCCTCAATGATTGC | A572237Hi | +T*+T*+C*C*T*C*A*A*T*G*A*T*T*G*C*A*+ | 60742 | 19 |
| | AAAG | | A*+A*+G | | |
| 2240 | TCCTCAATGATTGCA | A572238Hi | +T*+C*+C*T*C*A*A*T*G*A*T*T*G*C*A*+A* | 60742 | 18 |
| | AAG | | +A*+G | | |
| 2241 | TTTCCTCAATGATTG | A572239Hi | +T*+T*+T*C*C*T*C*A*A*T*G*A*T*T*G*C*+ | 60743 | 19 |
| | CAAA | | A*+A*+A | | |
| 2242 | ATGTCTCAGTAATTT | A572240Hi | +A*+T*+G*T*C*T*C*A*G*T*A*A*T*T*T*C*+ | 60947 | 19 |
| | CCAA | | C*+A*+A | | |
| 2243 | GACTAGTTTAAGTAA | A572241Hi | +G*+A*+C*T*A*G*T*T*T*A*A*G*T*A*A*T*+ | 60981 | 19 |
| | TGAA | | G*+A*+A | | |
| 2244 | AGACTAGTTTAAGTA | A572242Hi | +A*+G*+A*C*T*A*G*T*T*T*A*A*G*T*A*A*+ | 60982 | 19 |
| | ATGA | | T*+G*+A | | |
| 2245 | AGACTAGTTTAAGTA | A572243Hi | +A*+G*+A*C*T*A*G*T*T*T*A*A*G*T*A*+A* | 60983 | 18 |
| | ATG | | +T*+G | | |
| 2246 | AGATGTGTCAATGGT | A572244Hi | +A*+G*+A*T*G*T*G*T*C*A*A*T*G*G*T*+A* | 61121 | 18 |
| | AAA | | +A*+A | | |
| 2247 | CTTTACTCAGACCAG | A572245Hi | +C*+T*+T*T*A*C*T*C*A*G*A*C*C*A*G*+C* | 61205 | 18 |
| | CTG | | +T*+G | | |
| 2248 | TTCTTTACTCAGACC | A572246Hi | +T*+T*+C*T*T*T*A*C*T*C*A*G*A*C*C*A*+ | 61206 | 19 |
| | AGCT | | G*+C*+T | | |
| 2249 | GTGTTCTTTACTCAG | A572247Hi | +G*+T*+G*T*T*C*T*T*T*A*C*T*C*A*G*A*+ | 61209 | 19 |
| | ACCA | | C*+C*+A | | |
| 2250 | AGTGTTCTTTACTCA | A572248Hi | +A*+G*+T*G*T*T*C*T*T*T*A*C*T*C*A*G*+ | 61210 | 19 |
| | GACC | | A*+C*+C | | |
| 2251 | AGTGTTCTTTACTCA | A572249Hi | +A*+G*+T*G*T*T*C*T*T*T*A*C*T*C*A*+G* | 61211 | 18 |
| | GAC | | +A*+C | | |
| 2252 | GTATGCTCATGTATC | A572250Hi | +G*+T*+A*T*G*C*T*C*A*T*G*T*A*T*C*T*+ | 61450 | 19 |
| | TACA | | A*+C*+A | | |
| 2253 | GTATGCTCATGTATC | A572251Hi | +G*+T*+A*T*G*C*T*C*A*T*G*T*A*T*C*+T* | 61451 | 18 |
| | TAC | | +A*+C | | |
| 2254 | AGTATGCTCATGTAT | A572252Hi | +A*+G*+T*A*T*G*C*T*C*A*T*G*T*A*T*C*+ | 61451 | 19 |
| | CTAC | | T*+A*+C | | |
| 2255 | GTACAATATCCAGTG | A572253Hi | +G*+T*+A*C*A*A*T*A*T*C*C*A*G*T*G*+A* | 61638 | 18 |
| | ATG | | +T*+G | | |
| 2256 | TGTAGTACAATATCC | A572254Hi | +T*+G*+T*A*G*T*A*C*A*A*T*A*T*C*C*A*+ | 61641 | 19 |
| | AGTG | | G*+T*+G | | |
| 2257 | GTATCATGTAGTACA | A572255Hi | +G*+T*+A*T*C*A*T*G*T*A*G*T*A*C*A*A*+ | 61647 | 19 |
| | ATAT | | T*+A*+T | | |
| 2258 | GTATCATGTAGTACA | A572256Hi | +G*+T*+A*T*C*A*T*G*T*A*G*T*A*C*A*+A* | 61648 | 18 |
| | ATA | | +T*+A | | |
| 2259 | CAAATATTACTACAT | A572257Hi | +C*+A*+A*A*T*A*T*T*A*C*T*A*C*A*T*G*+ | 61671 | 19 |
| | GATA | | A*+T*+A | | |
| 2260 | GAGTCCTCATTGTTC | A572258Hi | +G*+A*+G*T*C*C*T*C*A*T*T*G*T*T*C*T*+ | 61734 | 19 |
| | TACA | | A*+C*+A | | |
| 2261 | TCTCACAACTTCATTA | A572259Hi | +T*+C*+T*C*A*C*A*A*C*T*T*C*A*T*T*A*+ | 61788 | 19 |
| | CAT | | C*+A*+T | | |
| 2262 | TGATTCATTATGACT | A572260Hi | +T*+G*+A*T*T*C*A*T*T*A*T*G*A*C*T*A*+ | 61840 | 19 |
| | ATTG | | T*+T*+G | | |
| 2263 | GAGTGATTCATTATG | A572261Hi | +G*+A*+G*T*G*A*T*T*C*A*T*T*A*T*G*A*+ | 61843 | 19 |
| | ACTA | | C*+T*+A | | |
| 2264 | TGAGTGATTCATTAT | A572262Hi | +T*+G*+A*G*T*G*A*T*T*C*A*T*T*A*T*+G* | 61845 | 18 |
| | GAC | | +A*+C | | |
| 2265 | GCTGAGTGATTCATT | A572263Hi | +G*+C*+T*G*A*G*T*G*A*T*T*C*A*T*T*+A* | 61847 | 18 |
| | ATG | | +T*+G | | |
| 2266 | TTATAAAGTTTGCTG | A572264Hi | +T*+T*+A*T*A*A*A*G*T*T*T*G*C*T*G*A*+ | 61857 | 19 |
| | AGTG | | G*+T*+G | | |
| 2267 | AGAAATGCCATTTGT | A572265Hi | +A*+G*+A*A*A*T*G*C*C*A*T*T*T*G*T*G*+ | 62035 | 19 |
| | GAGT | | A*+G*+T | | |
| 2268 | TGTTCAAGTCATAGA | A572266Hi | +T*+G*+T*T*C*A*A*G*T*C*A*T*A*G*A*A*+ | 62047 | 19 |
| | AATG | | A*+T*+G | | |
| 2269 | AGAAATCAACAATTA | A572267Hi | +A*+G*+A*A*A*T*C*A*A*C*A*A*T*T*A*T*+ | 62308 | 19 |
| | TGCA | | G*+C*+A | | |
| 2270 | TAGAAATCAACAATT | A572268Hi | +T*+A*+G*A*A*A*T*C*A*A*C*A*A*T*T*A*+ | 62309 | 19 |
| | ATGC | | T*+G*+C | | |
| 2271 | GCTAGTGTAGATGCT | A572269H | +G*+C*+T*A*G*T*G*T*A*G*A*T*G*C*T*A*+ | 62355 | 19 |
| | ATAT | | T*+A*+T | | |
| 2272 | TGCTAGTGTAGATGC | A572270H | +T*+G*+C*T*A*G*T*G*T*A*G*A*T*G*C*T*+ | 62356 | 19 |
| | TATA | | A*+T*+A | | |
| 2273 | TTGCTAGTGTAGATG | A572271H | +T*+T*+G*C*T*A*G*T*G*T*A*G*A*T*G*C*+ | 62357 | 19 |
| | CTAT | | T*+A*+T | | |
| 2274 | CTTGCTAGTGTAGAT | A572272H | +C*+T*+T*G*C*T*A*G*T*G*T*A*G*A*T*G*+ | 62358 | 19 |
| | GCTA | | C*+T*+A | | |
| 2275 | TCTTGCTAGTGTAGA | A572273H | +T*+C*+T*T*G*C*T*A*G*T*G*T*A*G*A*T*+ | 62359 | 19 |
| | TGCT | | G*+C*+T | | |
| 2276 | TCTTGCTAGTGTAGA | A572274H | +T*+C*+T*T*G*C*T*A*G*T*G*T*A*G*A*+T* | 62360 | 18 |
| | TGC | | +G*+C | | |
| 2277 | TTCTTGCTAGTGTAG | A572275H | +T*+T*+C*T*T*G*C*T*A*G*T*G*T*A*G*A*+ | 62360 | 19 |
| | ATGC | | T*+G*+C | | |
| 2278 | TCACCATTCTTGCTA | A572276H | +T*+C*+A*C*C*A*T*T*C*T*T*G*C*T*A*G*+ | 62366 | 19 |
| | GTGT | | T*+G*+T | | |
| 2279 | TCACCATTCTTGCTA | A572277H | +T*+C*+A*C*C*A*T*T*C*T*T*G*C*T*A*+G* | 62367 | 18 |
| | GTG | | +T*+G | | |
| 2280 | ATCACCATTCTTGCT | A572278H | +A*+T*+C*A*C*C*A*T*T*C*T*T*G*C*T*A*+ | 62367 | 19 |
| | AGTG | | G*+T*+G | | |
| 2281 | GATCACCATTCTTGC | A572279H | +G*+A*+T*C*A*C*C*A*T*T*C*T*T*G*C*T*+ | 62368 | 19 |
| | TAGT | | A*+G*+T | | |
| 2282 | CTGATCACCATTCTT | A572280H | +C*+T*+G*A*T*C*A*C*C*A*T*T*C*T*T*G*+ | 62370 | 19 |
| | GCTA | | C*+T*+A | | |
| 2283 | TCTGATCACCATTCTT | A572281H | +T*+C*+T*G*A*T*C*A*C*C*A*T*T*C*T*T*+ | 62371 | 19 |
| | GCT | | G*+C*+T | | |
| 2284 | TCTGATCACCATTCTT | A572282H | +T*+C*+T*G*A*T*C*A*C*C*A*T*T*C*T*+T* | 62372 | 18 |
| | GC | | +G*+C | | |
| 2285 | ATCTGATCACCATTC | A572283H | +A*+T*+C*T*G*A*T*C*A*C*C*A*T*T*C*T*+ | 62372 | 19 |
| | TTGC | | T*+G*+C | | |
| 2286 | GATATCTGATCACCA | A572284H | +G*+A*+T*A*T*C*T*G*A*T*C*A*C*C*A*T*+ | 62375 | 19 |
| | TTCT | | T*+C*+T | | |
| 2287 | TGATATCTGATCACC | A572285H | +T*+G*+A*T*A*T*C*T*G*A*T*C*A*C*C*A*+ | 62376 | 19 |
| | ATTC | | T*+T*+C | | |
| 2288 | TCTGATATCTGATCA | A572286H | +T*+C*+T*G*A*T*A*T*C*T*G*A*T*C*A*C*+ | 62378 | 19 |
| | CCAT | | C*+A*+T | | |
| 2289 | CATCTGATATCTGAT | A572287H | +C*+A*+T*C*T*G*A*T*A*T*C*T*G*A*T*C*+ | 62380 | 19 |
| | CACC | | A*+C*+C | | |
| 2290 | TTTCATCTGATATCTG | A572288H | +T*+T*+T*C*A*T*C*T*G*A*T*A*T*C*T*G*+ | 62383 | 19 |
| | ATC | | A*+T*+C | | |
| 2291 | ACTTTTCATCTGATAT | A572289H | +A*+C*+T*T*T*T*C*A*T*C*T*G*A*T*A*T*+ | 62386 | 19 |
| | CTG | | C*+T*+G | | |
| 2292 | TTAGACAGCACATCT | A572290H | +T*+T*+A*G*A*C*A*G*C*A*C*A*T*C*T*T*+ | 62448 | 19 |
| | TCAG | | C*+A*+G | | |
| 2293 | TTTAGACAGCACATC | A572291H | +T*+T*+T*A*G*A*C*A*G*C*A*C*A*T*C*T*+ | 62449 | 19 |
| | TTCA | | T*+C*+A | | |
| 2294 | AAATTTAGACAGCAC | A572292H | +A*+A*+A*T*T*T*A*G*A*C*A*G*C*A*C*A*+ | 62452 | 19 |
| | ATCT | | T*+C*+T | | |
| 2295 | AAATTTAGACAGCAC | A572293H | +A*+A*+A*T*T*T*A*G*A*C*A*G*C*A*C*+A* | 62453 | 18 |
| | ATC | | +T*+C | | |
| 2296 | CAAATTTAGACAGCA | A572294H | +C*+A*+A*A*T*T*T*A*G*A*C*A*G*C*A*C*+ | 62453 | 19 |
| | CATC | | A*+T*+C | | |
| 2297 | ATCAAATTTAGACAG | A572295H | +A*+T*+C*A*A*A*T*T*T*A*G*A*C*A*G*C*+ | 62455 | 19 |
| | CACA | | A*+C*+A | | |
| 2298 | ATCAAATTTAGACAG | A572296H | +A*+T*+C*A*A*A*T*T*T*A*G*A*C*A*G*+C* | 62456 | 18 |
| | CAC | | +A*+C | | |
| 2299 | CATCAAATTTAGACA | A572297H | +C*+A*+T*C*A*A*A*T*T*T*A*G*A*C*A*G*+ | 62456 | 19 |
| | GCAC | | C*+A*+C | | |
| 2300 | GGTCCATTCATCAAA | A572298H | +G*+G*+T*C*C*A*T*T*C*A*T*C*A*A*A*T*+ | 62464 | 19 |
| | TTTA | | T*+T*+A | | |
| 2301 | AGGTCCATTCATCAA | A572299H | +A*+G*+G*T*C*C*A*T*T*C*A*T*C*A*A*A*+ | 62465 | 19 |
| | ATTT | | T*+T*+T | | |
| 2302 | CATAGAAGAGTCAGG | A572300H | +C*+A*+T*A*G*A*A*G*A*G*T*C*A*G*G*A*+ | 62485 | 19 |
| | AATA | | A*+T*+A | | |
| 2303 | TCCAGTCTAGTCAGA | A572301Hi | +T*+C*+C*A*G*T*C*T*A*G*T*C*A*G*A*+G* | 62778 | 18 |
| | GTT | | +T*+T | | |
| 2304 | CGTTCCAGTCTAGTC | A572302Hi | +C*+G*+T*T*C*C*A*G*T*C*T*A*G*T*C*A*+ | 62780 | 19 |
| | AGAG | | G*+A*+G | | |
| 2305 | TCGTTCCAGTCTAGT | A572303Hi | +T*+C*+G*T*T*C*C*A*G*T*C*T*A*G*T*C*+ | 62781 | 19 |
| | CAGA | | A*+G*+A | | |
| 2306 | GAATCTCGTTCCAGT | A572304Hi | +G*+A*+A*T*C*T*C*G*T*T*C*C*A*G*T*+C* | 62787 | 18 |
| | CTA | | +T*+A | | |
| 2307 | TGGAATCTCGTTCCA | A572305Hi | +T*+G*+G*A*A*T*C*T*C*G*T*T*C*C*A*+G* | 62789 | 18 |
| | GTC | | +T*+C | | |
| 2308 | GTGGAATCTCGTTCC | A572306Hi | +G*+T*+G*G*A*A*T*C*T*C*G*T*T*C*+C*+A | 62791 | 17 |
| | AG | | *+G | | |
| 2309 | TTCAATCATACTATAT | A572307Hi | +T*+T*+C*A*A*T*C*A*T*A*C*T*A*T*A*T*+ | 63129 | 19 |
| | TAA | | T*+A*+A | | |
| 2310 | CTCAAATATCCTTATT | A572308Hi | +C*+T*+C*A*A*A*T*A*T*C*C*T*T*A*T*T*+ | 63160 | 19 |
| | CTA | | C*+T*+A | | |
| 2311 | TCTCAAATATCCTTAT | A572309Hi | +T*+C*+T*C*A*A*A*T*A*T*C*C*T*T*A*T*+ | 63161 | 19 |
| | TCT | | T*+C*+T | | |
| 2312 | GGTTTGACTTGAGAC | A572310Hi | +G*+G*+T*T*T*G*A*C*T*T*G*A*G*A*C*A*+ | 63209 | 19 |
| | ACAA | | C*+A*+A | | |
| 2313 | TGGTTTGACTTGAGA | A572311Hi | +T*+G*+G*T*T*T*G*A*C*T*T*G*A*G*A*C*+ | 63210 | 19 |
| | CACA | | A*+C*+A | | |
| 2314 | GGTTTGACTTGAGAC | A572312Hi | +G*+G*+T*T*T*G*A*C*T*T*G*A*G*A*C*+A* | 63210 | 18 |
| | ACA | | +C*+A | | |
| 2315 | TGGTTTGACTTGAGA | A572313Hi | +T*+G*+G*T*T*T*G*A*C*T*T*G*A*G*A*+C* | 63211 | 18 |
| | CAC | | +A*+C | | |
| 2316 | CAGTGAAGAGGCATG | A572314Hi | +C*+A*+G*T*G*A*A*G*A*G*G*C*A*T*G*A* | 63390 | 19 |
| | AAGA | | +A*+G*+A | | |
| 2317 | GTCAGGAAGCAAATG | A572315Hi | +G*+T*+C*A*G*G*A*A*G*C*A*A*A*T*G*A*+ | 63445 | 19 |
| | ATTG | | T*+T*+G | | |
| 2318 | CACAACTTCTATCTA | A572316Hi | +C*+A*+C*A*A*C*T*T*C*T*A*T*C*T*A*G*+ | 63803 | 19 |
| | GAAT | | A*+A*+T | | |
| 2319 | CTGTCACAACTTCTA | A572317Hi | +C*+T*+G*T*C*A*C*A*A*C*T*T*C*T*A*T*+ | 63807 | 19 |
| | TCTA | | C*+T*+A | | |
| 2320 | TGTCACAACTTCTAT | A572318Hi | +T*+G*+T*C*A*C*A*A*C*T*T*C*T*A*T*+C* | 63807 | 18 |
| | CTA | | +T*+A | | |
| 2321 | ACCTGTCACAACTTC | A572319Hi | +A*+C*+C*T*G*T*C*A*C*A*A*C*T*T*C*T*+ | 63809 | 19 |
| | TATC | | A*+T*+C | | |
| 2322 | ACCTGTCACAACTTC | A572320Hi | +A*+C*+C*T*G*T*C*A*C*A*A*C*T*T*C*+T* | 63810 | 18 |
| | TAT | | +A*+T | | |
| 2323 | AATATTCACAAATTTA | A572321Hi | +A*+A*+T*A*T*T*C*A*C*A*A*A*T*T*T*A*+ | 63990 | 19 |
| | GGC | | G*+G*+C | | |
| 2324 | CCATTAATTCAATGA | A572322Hi | +C*+C*+A*T*T*A*A*T*T*C*A*A*T*G*A*G*+ | 64177 | 19 |
| | GGAA | | G*+A*+A | | |
| 2325 | TTCCATTAATTCAATG | A572323Hi | +T*+T*+C*C*A*T*T*A*A*T*T*C*A*A*T*G*+ | 64179 | 19 |
| | AGG | | A*+G*+G | | |
| 2326 | CATACCTAAATAGTC | A572324Hi | +C*+A*+T*A*C*C*T*A*A*A*T*A*G*T*C*A*+ | 64212 | 19 |
| | AATT | | A*+T*+T | | |
| 2327 | CCATACCTAAATAGT | A572325Hi | +C*+C*+A*T*A*C*C*T*A*A*A*T*A*G*T*C*+ | 64213 | 19 |
| | CAAT | | A*+A*+T | | |
| 2328 | CATACCTAAATAGTC | A572326Hi | +C*+A*+T*A*C*C*T*A*A*A*T*A*G*T*C*+A* | 64213 | 18 |
| | AAT | | +A*+T | | |
| 2329 | AACCATACCTAAATA | A572327Hi | +A*+A*+C*C*A*T*A*C*C*T*A*A*A*T*A*G*+ | 64215 | 19 |
| | GTCA | | T*+C*+A | | |
| 2330 | AGAACCATACCTAAA | A572328Hi | +A*+G*+A*A*C*C*A*T*A*C*C*T*A*A*A*T*+ | 64217 | 19 |
| | TAGT | | A*+G*+T | | |
| 2331 | GAACCATACCTAAAT | A572329Hi | +G*+A*+A*C*C*A*T*A*C*C*T*A*A*A*T*+A* | 64217 | 18 |
| | AGT | | +G*+T | | |
| 2332 | TAAGAATAAGGTATA | A572330Hi | +T*+A*+A*G*A*A*T*A*A*G*G*T*A*T*A*A*+ | 64264 | 19 |
| | AATC | | A*+T*+C | | |
| 2333 | AAAGAACACCAGAGG | A572331Hi | +A*+A*+A*G*A*A*C*A*C*C*A*G*A*G*G*A* | 64345 | 19 |
| | ACTT | | +C*+T*+T | | |
| 2334 | TTGAACTATATATCA | A572332Hi | +T*+T*+G*A*A*C*T*A*T*A*T*A*T*C*A*C*+ | 64627 | 19 |
| | CATT | | A*+T*+T | | |
| 2335 | ATACAACACACTGTT | A572333Hi | +A*+T*+A*C*A*A*C*A*C*A*C*T*G*T*T*A*+ | 64661 | 19 |
| | AGAA | | G*+A*+A | | |
| 2336 | ATACAACACACTGTT | A572334Hi | +A*+T*+A*C*A*A*C*A*C*A*C*T*G*T*T*+A* | 64662 | 18 |
| | AGA | | +G*+A | | |
| 2337 | AATACAACACACTGT | A572335Hi | +A*+A*+T*A*C*A*A*C*A*C*A*C*T*G*T*T*+ | 64662 | 19 |
| | TAGA | | A*+G*+A | | |
| 2338 | ATCCAATACAACACA | A572336Hi | +A*+T*+C*C*A*A*T*A*C*A*A*C*A*C*A*C*+ | 64666 | 19 |
| | CTGT | | T*+G*+T | | |
| 2339 | CTATAATAAGGTACT | A572337Hi | +C*+T*+A*T*A*A*T*A*A*G*G*T*A*C*T*+G* | 64822 | 18 |
| | GGA | | +G*+A | | |
| 2340 | TCTATAATAAGGTAC | A572338Hi | +T*+C*+T*A*T*A*A*T*A*A*G*G*T*A*C*T*+ | 64822 | 19 |
| | TGGA | | G*+G*+A | | |
| 2341 | TCTATAATAAGGTAC | A572339Hi | +T*+C*+T*A*T*A*A*T*A*A*G*G*T*A*C*+T* | 64823 | 18 |
| | TGG | | +G*+G | | |
| 2342 | ATCTATAATAAGGTA | A572340Hi | +A*+T*+C*T*A*T*A*A*T*A*A*G*G*T*A*+C* | 64824 | 18 |
| | CTG | | +T*+G | | |
| 2343 | CCTCTAAGTTTGGAA | A572341Hi | +C*+C*+T*C*T*A*A*G*T*T*T*G*G*A*A*A*+ | 65006 | 19 |
| | ACCA | | C*+C*+A | | |
| 2344 | TGTCATAATTAGTGA | A572342Hi | +T*+G*+T*C*A*T*A*A*T*T*A*G*T*G*A*T*+ | 65040 | 19 |
| | TTAG | | T*+A*+G | | |
| 2345 | AATGTCATAATTAGT | A572343Hi | +A*+A*+T*G*T*C*A*T*A*A*T*T*A*G*T*G*+ | 65042 | 19 |
| | GATT | | A*+T*+T | | |
| 2346 | TAATGTCATAATTAG | A572344Hi | +T*+A*+A*T*G*T*C*A*T*A*A*T*T*A*G*T*+ | 65043 | 19 |
| | TGAT | | G*+A*+T | | |
| 2347 | TACCTCAGCAGATGC | A572345Hi | +T*+A*+C*C*T*C*A*G*C*A*G*A*T*G*C*T*+ | 65164 | 19 |
| | TGAA | | G*+A*+A | | |
| 2348 | TATGAATACCTCAGC | A572346Hi | +T*+A*+T*G*A*A*T*A*C*C*T*C*A*G*C*A*+ | 65170 | 19 |
| | AGAT | | G*+A*+T | | |
| 2349 | GGATAATGTACCATA | A572347Hi | +G*+G*+A*T*A*A*T*G*T*A*C*C*A*T*A*+A* | 65279 | 18 |
| | ACC | | +C*+C | | |
| 2350 | GGATAATGTACCATA | A572348Hi | +G*+G*+A*T*A*A*T*G*T*A*C*C*A*T*+A*+A | 65280 | 17 |
| | AC | | *+C | | |
| 2351 | AGGATAATGTACCAT | A572349Hi | +A*+G*+G*A*T*A*A*T*G*T*A*C*C*A*T*+A* | 65280 | 18 |
| | AAC | | +A*+C | | |
| 2352 | ACTAATATCTCTCAC | A572350Hi | +A*+C*+T*A*A*T*A*T*C*T*C*T*C*A*C*A*+ | 65347 | 19 |
| | AGAT | | G*+A*+T | | |
| 2353 | CCTTTGCTGGTGAAG | A572351Hi | +C*+C*+T*T*T*G*C*T*G*G*T*G*A*A*G*T*+ | 65494 | 19 |
| | TCTA | | C*+T*+A | | |
| 2354 | GGCCTTTGCTGGTGA | A572352Hi | +G*+G*+C*C*T*T*T*G*C*T*G*G*T*G*A*A*+ | 65496 | 19 |
| | AGTC | | G*+T*+C | | |
| 2355 | AGATGGCCTTTGCTG | A572353Hi | +A*+G*+A*T*G*G*C*C*T*T*T*G*C*T*G*G*+ | 65500 | 19 |
| | GTGA | | T*+G*+A | | |
| 2356 | TAGATGGCCTTTGCT | A572354Hi | +T*+A*+G*A*T*G*G*C*C*T*T*T*G*C*T*G*+ | 65501 | 19 |
| | GGTG | | G*+T*+G | | |
| 2357 | ATCAAAGTTATTAAA | A572355Hi | +A*+T*+C*A*A*A*G*T*T*A*T*T*A*A*A*T*+ | 65556 | 19 |
| | TTCA | | T*+C*+A | | |
| 2358 | CATACATCTATCAAA | A572356Hi | +C*+A*+T*A*C*A*T*C*T*A*T*C*A*A*A*G*+ | 65565 | 19 |
| | GTTA | | T*+T*+A | | |
| 2359 | CATACATCTATCAAA | A572357Hi | +C*+A*+T*A*C*A*T*C*T*A*T*C*A*A*A*+G* | 65566 | 18 |
| | GTT | | +T*+T | | |
| 2360 | CATATTCCTTGAAAG | A572358Hi | +C*+A*+T*A*T*T*C*C*T*T*G*A*A*A*G*T*+ | 65629 | 19 |
| | TTAC | | T*+A*+C | | |
| 2361 | AATCACTAACAGGAT | A572359Hi | +A*+A*+T*C*A*C*T*A*A*C*A*G*G*A*T*A*+ | 65705 | 19 |
| | AATT | | A*+T*+T | | |
| 2362 | TAATTAAATCACTAA | A572360Hi | +T*+A*+A*T*T*A*A*A*T*C*A*C*T*A*A*C*+ | 65711 | 19 |
| | CAGG | | A*+G*+G | | |
| 2363 | GGATAATTAAATCAC | A572361Hi | +G*+G*+A*T*A*A*T*T*A*A*A*T*C*A*C*T*+ | 65714 | 19 |
| | TAAC | | A*+A*+C | | |
| 2364 | GCTTCCAAATTAAGA | A572362Hi | +G*+C*+T*T*C*C*A*A*A*T*T*A*A*G*A*A*+ | 65870 | 19 |
| | ATCT | | T*+C*+T | | |
| 2365 | GACAGCCTTCTAAAT | A572363Hi | +G*+A*+C*A*G*C*C*T*T*C*T*A*A*A*T*+A* | 66490 | 18 |
| | AAC | | +A*+C | | |
| 2366 | TAGAAATCATCAGAG | A572364Hi | +T*+A*+G*A*A*A*T*C*A*T*C*A*G*A*G*T*+ | 66589 | 19 |
| | TAGG | | A*+G*+G | | |
| 2367 | TGGCCTATTGATTTG | A572365Hi | +T*+G*+G*C*C*T*A*T*T*G*A*T*T*T*G*+A* | 66709 | 18 |
| | ACC | | +C*+C | | |
| 2368 | TTGGCCTATTGATTT | A572366Hi | +T*+T*+G*G*C*C*T*A*T*T*G*A*T*T*T*G*+ | 66709 | 19 |
| | GACC | | A*+C*+C | | |
| 2369 | CCAAACAATTTGGCC | A572367Hi | +C*+C*+A*A*A*C*A*A*T*T*T*G*G*C*C*+T* | 66719 | 18 |
| | TAT | | +A*+T | | |
| 2370 | CCTCAATTACAACTT | A572368Hi | +C*+C*+T*C*A*A*T*T*A*C*A*A*C*T*T*C*+ | 66805 | 19 |
| | CAAT | | A*+A*+T | | |
| 2371 | TCCTCAATTACAACTT | A572369Hi | +T*+C*+C*T*C*A*A*T*T*A*C*A*A*C*T*T*+ | 66806 | 19 |
| | CAA | | C*+A*+A | | |
| 2372 | ATCCTCAATTACAAC | A572370Hi | +A*+T*+C*C*T*C*A*A*T*T*A*C*A*A*C*T*+ | 66807 | 19 |
| | TTCA | | T*+C*+A | | |
| 2373 | ATCCTCAATTACAAC | A572371Hi | +A*+T*+C*C*T*C*A*A*T*T*A*C*A*A*C*+T* | 66808 | 18 |
| | TTC | | +T*+C | | |
| 2374 | CCAACAAATCCTCAA | A572372Hi | +C*+C*+A*A*C*A*A*A*T*C*C*T*C*A*A*T*+ | 66814 | 19 |
| | TTAC | | T*+A*+C | | |
| 2375 | TACAAAGCATAAACA | A572373Hi | +T*+A*+C*A*A*A*G*C*A*T*A*A*A*C*A*G*+ | 66848 | 19 |
| | GATA | | A*+T*+A | | |
| 2376 | TACTAACTGCAAACT | A572374Hi | +T*+A*+C*T*A*A*C*T*G*C*A*A*A*C*T*+T* | 66970 | 18 |
| | TAG | | +A*+G | | |
| 2377 | TTTCCTGAGATGAGG | A572375Hi | +T*+T*+T*C*C*T*G*A*G*A*T*G*A*G*G*A*+ | 67129 | 19 |
| | AGAT | | G*+A*+T | | |
| 2378 | AGCACATTATTTGTA | A572376Hi | +A*+G*+C*A*C*A*T*T*A*T*T*T*G*T*A*C*+ | 67457 | 19 |
| | CAAG | | A*+A*+G | | |
| 2379 | GTGAAATGTCTATTA | A572377Hi | +G*+T*+G*A*A*A*T*G*T*C*T*A*T*T*A*G*+ | 67471 | 19 |
| | GCAC | | C*+A*+C | | |
| 2380 | TGAAATGTCTATTAG | A572378Hi | +T*+G*+A*A*A*T*G*T*C*T*A*T*T*A*G*+C* | 67471 | 18 |
| | CAC | | +A*+C | | |
| 2381 | TGTGAAATGTCTATT | A572379Hi | +T*+G*+T*G*A*A*A*T*G*T*C*T*A*T*T*A*+ | 67472 | 19 |
| | AGCA | | G*+C*+A | | |
| 2382 | ATATTCAGTATAAAT | A572380Hi | +A*+T*+A*T*T*C*A*G*T*A*T*A*A*A*T*C*+ | 67654 | 19 |
| | CCTG | | C*+T*+G | | |
| 2383 | AATATTCAGTATAAA | A572381Hi | +A*+A*+T*A*T*T*C*A*G*T*A*T*A*A*A*T*+ | 67655 | 19 |
| | TCCT | | C*+C*+T | | |
| 2384 | ACAATAAATTTGGCT | A572382Hi | +A*+C*+A*A*T*A*A*A*T*T*T*G*G*C*T*G*+ | 67899 | 19 |
| | GGCA | | G*+C*+A | | |
| 2385 | AACACAATAAATTTG | A572383Hi | +A*+A*+C*A*C*A*A*T*A*A*A*T*T*T*G*G*+ | 67902 | 19 |
| | GCTG | | C*+T*+G | | |
| 2386 | TTTATGGAATGGTAT | A572384Hi | +T*+T*+T*A*T*G*G*A*A*T*G*G*T*A*T*T*+ | 67924 | 19 |
| | TAAA | | A*+A*+A | | |
| 2387 | AATTTCATGTTTCATG | A572385Hi | +A*+A*+T*T*T*C*A*T*G*T*T*T*C*A*T*G*+ | 67946 | 19 |
| | GTT | | G*+T*+T | | |
| 2388 | GTTGTAATCACACAA | A572386Hi | +G*+T*+T*G*T*A*A*T*C*A*C*A*C*A*A*A*+ | 68001 | 19 |
| | ATTA | | T*+T*+A | | |
| 2389 | ATGTTGTAATCACAC | A572387Hi | +A*+T*+G*T*T*G*T*A*A*T*C*A*C*A*C*A*+ | 68003 | 19 |
| | AAAT | | A*+A*+T | | |
| 2390 | ACTGCTTTCTGTTTCT | A572388Hi | +A*+C*+T*G*C*T*T*T*C*T*G*T*T*T*C*T*+ | 68125 | 19 |
| | CTG | | C*+T*+G | | |
| 2391 | TCAGAGGAGCTCATA | A572389Hi | +T*+C*+A*G*A*G*G*A*G*C*T*C*A*T*A*G*+ | 68234 | 19 |
| | GTCC | | T*+C*+C | | |
| 2392 | TTACAGACTAGACAC | A572390Hi | +T*+T*+A*C*A*G*A*C*T*A*G*A*C*A*C*+T* | 68298 | 18 |
| | TGG | | +G*+G | | |
| 2393 | CTCAATCATAAGATC | A572391Hi | +C*+T*+C*A*A*T*C*A*T*A*A*G*A*T*C*+C* | 68483 | 18 |
| | CTT | | +T*+T | | |
| 2394 | TCACAAGAAATGAGC | A572392H | +T*+C*+A*C*A*A*G*A*A*A*T*G*A*G*C*C*+ | 68554 | 19 |
| | CTTC | | T*+T*+C | | |
| 2395 | TTCACAAGAAATGAG | A572393H | +T*+T*+C*A*C*A*A*G*A*A*A*T*G*A*G*C*+ | 68555 | 19 |
| | CCTT | | C*+T*+T | | |
| 2396 | TTCACAAGAAATGAG | A572394H | +T*+T*+C*A*C*A*A*G*A*A*A*T*G*A*G*+C* | 68556 | 18 |
| | CCT | | +C*+T | | |
| 2397 | TTTCACAAGAAATGA | A572395H | +T*+T*+T*C*A*C*A*A*G*A*A*A*T*G*A*G*+ | 68556 | 19 |
| | GCCT | | C*+C*+T | | |
| 2398 | TTTTCACAAGAAATG | A572396H | +T*+T*+T*T*C*A*C*A*A*G*A*A*A*T*G*A*+ | 68557 | 19 |
| | AGCC | | G*+C*+C | | |
| 2399 | TTTTTCACAAGAAAT | A572397H | +T*+T*+T*T*T*C*A*C*A*A*G*A*A*A*T*G*+ | 68558 | 19 |
| | GAGC | | A*+G*+C | | |
| 2400 | ATTCTCCTACACTAAT | A572398H | +A*+T*+T*C*T*C*C*T*A*C*A*C*T*A*A*T*+ | 68587 | 19 |
| | TGA | | T*+G*+A | | |
| 2401 | ATTCTCCTACACTAAT | A572399H | +A*+T*+T*C*T*C*C*T*A*C*A*C*T*A*A*+T* | 68588 | 18 |
| | TG | | +T*+G | | |
| 2402 | AATTCTCCTACACTA | A572400H | +A*+A*+T*T*C*T*C*C*T*A*C*A*C*T*A*A*+ | 68588 | 19 |
| | ATTG | | T*+T*+G | | |
| 2403 | CGGTAAAATTCTCCT | A572401H | +C*+G*+G*T*A*A*A*A*T*T*C*T*C*C*T*A*+ | 68594 | 19 |
| | ACAC | | C*+A*+C | | |
| 2404 | CGGTAAAATTCTCCT | A572402H | +C*+G*+G*T*A*A*A*A*T*T*C*T*C*C*T*+A* | 68595 | 18 |
| | ACA | | +C*+A | | |
| 2405 | TCGGTAAAATTCTCC | A572403H | +T*+C*+G*G*T*A*A*A*A*T*T*C*T*C*C*T*+ | 68595 | 19 |
| | TACA | | A*+C*+A | | |
| 2406 | TCGGTAAAATTCTCC | A572404H | +T*+C*+G*G*T*A*A*A*A*T*T*C*T*C*C*+T* | 68596 | 18 |
| | TAC | | +A*+C | | |
| 2407 | CTCGGTAAAATTCTC | A572405H | +C*+T*+C*G*G*T*A*A*A*A*T*T*C*T*C*C*+ | 68596 | 19 |
| | CTAC | | T*+A*+C | | |
| 2408 | CTCGGTAAAATTCTC | A572406H | +C*+T*+C*G*G*T*A*A*A*A*T*T*C*T*C*+C* | 68597 | 18 |
| | CTA | | +T*+A | | |
| 2409 | TCTCGGTAAAATTCT | A572407H | +T*+C*+T*C*G*G*T*A*A*A*A*T*T*C*T*C*+ | 68597 | 19 |
| | CCTA | | C*+T*+A | | |
| 2410 | ATCTCGGTAAAATTC | A572408H | +A*+T*+C*T*C*G*G*T*A*A*A*A*T*T*C*T*+ | 68598 | 19 |
| | TCCT | | C*+C*+T | | |
| 2411 | CGGCATCTCGGTAAA | A572409H | +C*+G*+G*C*A*T*C*T*C*G*G*T*A*A*A*A*+ | 68602 | 19 |
| | ATTC | | T*+T*+C | | |
| 2412 | CGGCATCTCGGTAAA | A572410H | +C*+G*+G*C*A*T*C*T*C*G*G*T*A*A*A*+A* | 68603 | 18 |
| | ATT | | +T*+T | | |
| 2413 | ACGGCATCTCGGTAA | A572411H | +A*+C*+G*G*C*A*T*C*T*C*G*G*T*A*A*A*+ | 68603 | 19 |
| | AATT | | A*+T*+T | | |
| 2414 | ATACGGCATCTCGGT | A572412H | +A*+T*+A*C*G*G*C*A*T*C*T*C*G*G*T*A*+ | 68605 | 19 |
| | AAAA | | A*+A*+A | | |
| 2415 | ATACGGCATCTCGGT | A572413H | +A*+T*+A*C*G*G*C*A*T*C*T*C*G*G*T*+A* | 68606 | 18 |
| | AAA | | +A*+A | | |
| 2416 | AATACGGCATCTCGG | A572414H | +A*+A*+T*A*C*G*G*C*A*T*C*T*C*G*G*T*+ | 68606 | 19 |
| | TAAA | | A*+A*+A | | |
| 2417 | ATACGGCATCTCGGT | A572415H | +A*+T*+A*C*G*G*C*A*T*C*T*C*G*G*+T*+A | 68607 | 17 |
| | AA | | *+A | | |
| 2418 | AATACGGCATCTCGG | A572416H | +A*+A*+T*A*C*G*G*C*A*T*C*T*C*G*G*+T* | 68607 | 18 |
| | TAA | | +A*+A | | |
| 2419 | TAATACGGCATCTCG | A572417H | +T*+A*+A*T*A*C*G*G*C*A*T*C*T*C*G*G*+ | 68607 | 19 |
| | GTAA | | T*+A*+A | | |
| 2420 | ATACGGCATCTCGGT | A572418H | +A*+T*+A*C*G*G*C*A*T*C*T*C*G*+G*+T*+ | 68608 | 16 |
| | A | | A | | |
| 2421 | AATACGGCATCTCGG | A572419H | +A*+A*+T*A*C*G*G*C*A*T*C*T*C*G*+G*+T | 68608 | 17 |
| | TA | | *+A | | |
| 2422 | TAATACGGCATCTCG | A572420H | +T*+A*+A*T*A*C*G*G*C*A*T*C*T*C*G*+G* | 68608 | 18 |
| | GTA | | +T*+A | | |
| 2423 | GTAATACGGCATCTC | A572421H | +G*+T*+A*A*T*A*C*G*G*C*A*T*C*T*C*G*+ | 68608 | 19 |
| | GGTA | | G*+T*+A | | |
| 2424 | TAATACGGCATCTCG | A572422H | +T*+A*+A*T*A*C*G*G*C*A*T*C*T*C*+G*+G | 68609 | 17 |
| | GT | | *+T | | |
| 2425 | GTAATACGGCATCTC | A572423H | +G*+T*+A*A*T*A*C*G*G*C*A*T*C*T*C*+G* | 68609 | 18 |
| | GGT | | +G*+T | | |
| 2426 | TGTAATACGGCATCT | A572424H | +T*+G*+T*A*A*T*A*C*G*G*C*A*T*C*T*C*+ | 68609 | 19 |
| | CGGT | | G*+G*+T | | |
| 2427 | TAATACGGCATCTCG | A572425H | +T*+A*+A*T*A*C*G*G*C*A*T*C*T*+C*+G*+ | 68610 | 16 |
| | G | | G | | |
| 2428 | GTAATACGGCATCTC | A572426H | +G*+T*+A*A*T*A*C*G*G*C*A*T*C*T*+C*+G | 68610 | 17 |
| | GG | | *+G | | |
| 2429 | TGTAATACGGCATCT | A572427H | +T*+G*+T*A*A*T*A*C*G*G*C*A*T*C*T*+C* | 68610 | 18 |
| | CGG | | +G*+G | | |
| 2430 | CAAATTTGGTGAGCA | A572428H | +C*+A*+A*A*T*T*T*G*G*T*G*A*G*C*A*+A* | 68629 | 18 |
| | ACG | | +C*+G | | |
| 2431 | GCAAATTTGGTGAGC | A572429H | +G*+C*+A*A*A*T*T*T*G*G*T*G*A*G*C*A*+ | 68629 | 19 |
| | AACG | | A*+C*+G | | |
| 2432 | CTTTGCAAATTTGGT | A572430H | +C*+T*+T*T*G*C*A*A*A*T*T*T*G*G*T*G*+ | 68633 | 19 |
| | GAGC | | A*+G*+C | | |
| 2433 | GTTATCAAGAGAATG | A572431Hi | +G*+T*+T*A*T*C*A*A*G*A*G*A*A*T*G*T*+ | 68710 | 19 |
| | TCAG | | C*+A*+G | | |
| 2434 | GTTATCAAGAGAATG | A572432Hi | +G*+T*+T*A*T*C*A*A*G*A*G*A*A*T*G*+T* | 68711 | 18 |
| | TCA | | +C*+A | | |
| 2435 | AGTTATCAAGAGAAT | A572433Hi | +A*+G*+T*T*A*T*C*A*A*G*A*G*A*A*T*+G* | 68712 | 18 |
| | GTC | | +T*+C | | |
| 2436 | CTAGCTTTGGCATAA | A572434Hi | +C*+T*+A*G*C*T*T*T*G*G*C*A*T*A*A*A*+ | 68731 | 19 |
| | ATTT | | T*+T*+T | | |
| 2437 | TTGTTCCTAGCTTTG | A572435Hi | +T*+T*+G*T*T*C*C*T*A*G*C*T*T*T*G*G*+ | 68737 | 19 |
| | GCAT | | C*+A*+T | | |
| 2438 | ATTGTTCCTAGCTTT | A572436Hi | +A*+T*+T*G*T*T*C*C*T*A*G*C*T*T*T*G*+ | 68738 | 19 |
| | GGCA | | G*+C*+A | | |
| 2439 | AATTGTTCCTAGCTTT | A572437Hi | +A*+A*+T*T*G*T*T*C*C*T*A*G*C*T*T*T*+ | 68739 | 19 |
| | GGC | | G*+G*+C | | |
| 2440 | ATTGTTCCTAGCTTT | A572438Hi | +A*+T*+T*G*T*T*C*C*T*A*G*C*T*T*T*+G* | 68739 | 18 |
| | GGC | | +G*+C | | |
| 2441 | ACCAATTGTTCCTAG | A572439Hi | +A*+C*+C*A*A*T*T*G*T*T*C*C*T*A*G*+C* | 68743 | 18 |
| | CTT | | +T*+T | | |
| 2442 | CCATCTTGAAGTGTA | A572440Hi | +C*+C*+A*T*C*T*T*G*A*A*G*T*G*T*A*+C* | 68924 | 18 |
| | CTG | | +T*+G | | |
| 2443 | ACCATCTTGAAGTGT | A572441Hi | +A*+C*+C*A*T*C*T*T*G*A*A*G*T*G*T*A*+ | 68924 | 19 |
| | ACTG | | C*+T*+G | | |
| 2444 | CTAGCTAAAGTGTTC | A572442Hi | +C*+T*+A*G*C*T*A*A*A*G*T*G*T*T*C*A*+ | 69018 | 19 |
| | ATGG | | T*+G*+G | | |
| 2445 | GATTTCTAGCTAAAG | A572443Hi | +G*+A*+T*T*T*C*T*A*G*C*T*A*A*A*G*+T* | 69024 | 18 |
| | TGT | | +G*+T | | |
| 2446 | CTGGTTTCTTAGAAA | A572444Hi | +C*+T*+G*G*T*T*T*C*T*T*A*G*A*A*A*T*+ | 69047 | 19 |
| | TCAC | | C*+A*+C | | |
| 2447 | TCTGGTTTCTTAGAA | A572445Hi | +T*+C*+T*G*G*T*T*T*C*T*T*A*G*A*A*A*+ | 69048 | 19 |
| | ATCA | | T*+C*+A | | |
| 2448 | CTGTCTGGTTTCTTA | A572446Hi | +C*+T*+G*T*C*T*G*G*T*T*T*C*T*T*A*G*+ | 69051 | 19 |
| | GAAA | | A*+A*+A | | |
| 2449 | ACTTCTGTCTGGTTT | A572447Hi | +A*+C*+T*T*C*T*G*T*C*T*G*G*T*T*T*C*+ | 69055 | 19 |
| | CTTA | | T*+T*+A | | |
| 2450 | TCAGTGTCCTTCAGA | A572448Hi | +T*+C*+A*G*T*G*T*C*C*T*T*C*A*G*A*C*+ | 69069 | 19 |
| | CTTC | | T*+T*+C | | |
| 2451 | TACTCCATCTTGTTCA | A572449Hi | +T*+A*+C*T*C*C*A*T*C*T*T*G*T*T*C*A*+ | 69082 | 19 |
| | GTG | | G*+T*+G | | |
| 2452 | CTACTCCATCTTGTTC | A572450Hi | +C*+T*+A*C*T*C*C*A*T*C*T*T*G*T*T*C*+ | 69083 | 19 |
| | AGT | | A*+G*+T | | |
| 2453 | TATGCTACTCCATCTT | A572451Hi | +T*+A*+T*G*C*T*A*C*T*C*C*A*T*C*T*T*+ | 69087 | 19 |
| | GTT | | G*+T*+T | | |
| 2454 | ATGAATTATATTATG | A572452Hi | +A*+T*+G*A*A*T*T*A*T*A*T*T*A*T*G*C*+ | 69098 | 19 |
| | CTAC | | T*+A*+C | | |
| 2455 | CAATGAATTATATTAT | A572453Hi | +C*+A*+A*T*G*A*A*T*T*A*T*A*T*T*A*T*+ | 69100 | 19 |
| | GCT | | G*+C*+T | | |
| 2456 | TTCAAAGTGAACCTG | A572454Hi | +T*+T*+C*A*A*A*G*T*G*A*A*C*C*T*G*A*+ | 69353 | 19 |
| | AGAA | | G*+A*+A | | |
| 2457 | CTCATTAACCAAACT | A572455Hi | +C*+T*+C*A*T*T*A*A*C*C*A*A*A*C*T*A*+ | 69551 | 19 |
| | ACTT | | C*+T*+T | | |
| 2458 | CTCATTAACCAAACT | A572456Hi | +C*+T*+C*A*T*T*A*A*C*C*A*A*A*C*T*+A* | 69552 | 18 |
| | ACT | | +C*+T | | |
| 2459 | ATGATCAAAAATGGG | A572457H | +A*+T*+G*A*T*C*A*A*A*A*A*T*G*G*G*+C* | 69849 | 18 |
| | CCC | | +C*+C | | |
| 2460 | CATGATCAAAAATGG | A572458H | +C*+A*+T*G*A*T*C*A*A*A*A*A*T*G*G*G*+ | 69849 | 19 |
| | GCCC | | C*+C*+C | | |
| 2461 | TCATGATCAAAAATG | A572459H | +T*+C*+A*T*G*A*T*C*A*A*A*A*A*T*G*G*+ | 69850 | 19 |
| | GGCC | | G*+C*+C | | |
| 2462 | GTAAATCTTCATGAT | A572460H | +G*+T*+A*A*A*T*C*T*T*C*A*T*G*A*T*C*+ | 69858 | 19 |
| | CAAA | | A*+A*+A | | |
| 2463 | CAGTAAATCTTCATG | A572461H | +C*+A*+G*T*A*A*A*T*C*T*T*C*A*T*G*A*+ | 69860 | 19 |
| | ATCA | | T*+C*+A | | |
| 2464 | TCAGTAAATCTTCAT | A572462H | +T*+C*+A*G*T*A*A*A*T*C*T*T*C*A*T*G*+ | 69861 | 19 |
| | GATC | | A*+T*+C | | |
| 2465 | AATCATCTGAAGATA | A572463H | +A*+A*+T*C*A*T*C*T*G*A*A*G*A*T*A*A*+ | 69894 | 19 |
| | ATAT | | T*+A*+T | | |
| 2466 | GAATCATCTGAAGAT | A572464H | +G*+A*+A*T*C*A*T*C*T*G*A*A*G*A*T*A*+ | 69895 | 19 |
| | AATA | | A*+T*+A | | |
| 2467 | TGAATCATCTGAAGA | A572465H | +T*+G*+A*A*T*C*A*T*C*T*G*A*A*G*A*T*+ | 69896 | 19 |
| | TAAT | | A*+A*+T | | |
| 2468 | GTGAATCATCTGAAG | A572466H | +G*+T*+G*A*A*T*C*A*T*C*T*G*A*A*G*A*+ | 69897 | 19 |
| | ATAA | | T*+A*+A | | |
| 2469 | AGTGAATCATCTGAA | A572467H | +A*+G*+T*G*A*A*T*C*A*T*C*T*G*A*A*G*+ | 69898 | 19 |
| | GATA | | A*+T*+A | | |
| 2470 | GAGTGAATCATCTGA | A572468H | +G*+A*+G*T*G*A*A*T*C*A*T*C*T*G*A*A*+ | 69899 | 19 |
| | AGAT | | G*+A*+T | | |
| 2471 | GAGTGAATCATCTGA | A572469H | +G*+A*+G*T*G*A*A*T*C*A*T*C*T*G*A*+A* | 69900 | 18 |
| | AGA | | +G*+A | | |
| 2472 | TGAGTGAATCATCTG | A572470H | +T*+G*+A*G*T*G*A*A*T*C*A*T*C*T*G*A*+ | 69900 | 19 |
| | AAGA | | A*+G*+A | | |
| 2473 | TGATTACATCCAAAT | A572471Hi | +T*+G*+A*T*T*A*C*A*T*C*C*A*A*A*T*A*+ | 69921 | 19 |
| | ACTT | | C*+T*+T | | |
| 2474 | TTATGATTACATCCA | A572472Hi | +T*+T*+A*T*G*A*T*T*A*C*A*T*C*C*A*A*+ | 69924 | 19 |
| | AATA | | A*+T*+A | | |
| 2475 | CTTATGATTACATCC | A572473Hi | +C*+T*+T*A*T*G*A*T*T*A*C*A*T*C*C*A*+ | 69925 | 19 |
| | AAAT | | A*+A*+T | | |
| 2476 | ATTTGAATAATTGGA | A572474Hi | +A*+T*+T*T*G*A*A*T*A*A*T*T*G*G*A*G*+ | 69984 | 19 |
| | GGAA | | G*+A*+A | | |
| 2477 | TCTATAATATTCAAG | A572475Hi | +T*+C*+T*A*T*A*A*T*A*T*T*C*A*A*G*A*+ | 70104 | 19 |
| | AAAG | | A*+A*+G | | |
| 2478 | CCAAACTATGGAGTT | A572476Hi | +C*+C*+A*A*A*C*T*A*T*G*G*A*G*T*T*T*+ | 70288 | 19 |
| | TACA | | A*+C*+A | | |
| 2479 | ACCAAACTATGGAGT | A572477Hi | +A*+C*+C*A*A*A*C*T*A*T*G*G*A*G*T*T*+ | 70289 | 19 |
| | TTAC | | T*+A*+C | | |
| 2480 | CTAGCAATCAGGCTT | A572478Hi | +C*+T*+A*G*C*A*A*T*C*A*G*G*C*T*T*T*+ | 70333 | 19 |
| | TCTT | | C*+T*+T | | |
| 2481 | CTAGCAATCAGGCTT | A572479Hi | +C*+T*+A*G*C*A*A*T*C*A*G*G*C*T*T*+T* | 70334 | 18 |
| | TCT | | +C*+T | | |
| 2482 | CTCCTAGCAATCAGG | A572480Hi | +C*+T*+C*C*T*A*G*C*A*A*T*C*A*G*G*+C* | 70337 | 18 |
| | CTT | | +T*+T | | |
| 2483 | CACCTCCTAGCAATC | A572481Hi | +C*+A*+C*C*T*C*C*T*A*G*C*A*A*T*C*A*+ | 70339 | 19 |
| | AGGC | | G*+G*+C | | |
| 2484 | CACCTCCTAGCAATC | A572482Hi | +C*+A*+C*C*T*C*C*T*A*G*C*A*A*T*C*+A* | 70340 | 18 |
| | AGG | | +G*+G | | |
| 2485 | TGAGCACCTCCTAGC | A572483Hi | +T*+G*+A*G*C*A*C*C*T*C*C*T*A*G*C*+A* | 70344 | 18 |
| | AAT | | +A*+T | | |
| 2486 | GCAAATAATTTAAAT | A572484Hi | +G*+C*+A*A*A*T*A*A*T*T*T*A*A*A*T*T*+ | 70377 | 19 |
| | TCAT | | C*+A*+T | | |
| 2487 | GATTGAAGTTTTGAT | A572485H | +G*+A*+T*T*G*A*A*G*T*T*T*T*G*A*T*G*+ | 70431 | 19 |
| | GACC | | A*+C*+C | | |
| 2488 | GATTGAAGTTTTGAT | A572486H | +G*+A*+T*T*G*A*A*G*T*T*T*T*G*A*T*+G* | 70432 | 18 |
| | GAC | | +A*+C | | |
| 2489 | GGATTGAAGTTTTGA | A572487H | +G*+G*+A*T*T*G*A*A*G*T*T*T*T*G*A*T*+ | 70432 | 19 |
| | TGAC | | G*+A*+C | | |
| 2490 | GGGATTGAAGTTTTG | A572488H | +G*+G*+G*A*T*T*G*A*A*G*T*T*T*T*G*A*+ | 70433 | 19 |
| | ATGA | | T*+G*+A | | |
| 2491 | ATGGGATTGAAGTTT | A572489H | +A*+T*+G*G*G*A*T*T*G*A*A*G*T*T*T*T*+ | 70435 | 19 |
| | TGAT | | G*+A*+T | | |
| 2492 | TATGGGATTGAAGTT | A572490H | +T*+A*+T*G*G*G*A*T*T*G*A*A*G*T*T*T*+ | 70436 | 19 |
| | TTGA | | T*+G*+A | | |
| 2493 | ATATGGGATTGAAGT | A572491H | +A*+T*+A*T*G*G*G*A*T*T*G*A*A*G*T*T*+ | 70437 | 19 |
| | TTTG | | T*+T*+G | | |
| 2494 | CATATGGGATTGAAG | A572492H | +C*+A*+T*A*T*G*G*G*A*T*T*G*A*A*G*T*+ | 70438 | 19 |
| | TTTT | | T*+T*+T | | |
| 2495 | GCCTCATATGGGATT | A572493H | +G*+C*+C*T*C*A*T*A*T*G*G*G*A*T*T*G*+ | 70442 | 19 |
| | GAAG | | A*+A*+G | | |
| 2496 | GCCTCATATGGGATT | A572494H | +G*+C*+C*T*C*A*T*A*T*G*G*G*A*T*T*+G* | 70443 | 18 |
| | GAA | | +A*+A | | |
| 2497 | TGCCTCATATGGGAT | A572495H | +T*+G*+C*C*T*C*A*T*A*T*G*G*G*A*T*T*+ | 70443 | 19 |
| | TGAA | | G*+A*+A | | |
| 2498 | TGCCTCATATGGGAT | A572496H | +T*+G*+C*C*T*C*A*T*A*T*G*G*G*A*T*+T* | 70444 | 18 |
| | TGA | | +G*+A | | |
| 2499 | ATGCCTCATATGGGA | A572497H | +A*+T*+G*C*C*T*C*A*T*A*T*G*G*G*A*T*+ | 70444 | 19 |
| | TTGA | | T*+G*+A | | |
| 2500 | AATGCCTCATATGGG | A572498H | +A*+A*+T*G*C*C*T*C*A*T*A*T*G*G*G*A*+ | 70445 | 19 |
| | ATTG | | T*+T*+G | | |
| 2501 | GAATGCCTCATATGG | A572499H | +G*+A*+A*T*G*C*C*T*C*A*T*A*T*G*G*G*+ | 70446 | 19 |
| | GATT | | A*+T*+T | | |
| 2502 | GAATGCCTCATATGG | A572500H | +G*+A*+A*T*G*C*C*T*C*A*T*A*T*G*G*+G* | 70447 | 18 |
| | GAT | | +A*+T | | |
| 2503 | TGAATGCCTCATATG | A572501H | +T*+G*+A*A*T*G*C*C*T*C*A*T*A*T*G*G*+ | 70447 | 19 |
| | GGAT | | G*+A*+T | | |
| 2504 | TCTGAATGCCTCATA | A572502H | +T*+C*+T*G*A*A*T*G*C*C*T*C*A*T*A*T*+ | 70449 | 19 |
| | TGGG | | G*+G*+G | | |
| 2505 | GTCTGAATGCCTCAT | A572503H | +G*+T*+C*T*G*A*A*T*G*C*C*T*C*A*T*A*+ | 70450 | 19 |
| | ATGG | | T*+G*+G | | |
| 2506 | TGTCTGAATGCCTCA | A572504H | +T*+G*+T*C*T*G*A*A*T*G*C*C*T*C*A*T*+ | 70451 | 19 |
| | TATG | | A*+T*+G | | |
| 2507 | TGTCTGAATGCCTCA | A572505H | +T*+G*+T*C*T*G*A*A*T*G*C*C*T*C*A*+T* | 70452 | 18 |
| | TAT | | +A*+T | | |
| 2508 | CTGTCTGAATGCCTC | A572506H | +C*+T*+G*T*C*T*G*A*A*T*G*C*C*T*C*A*+ | 70452 | 19 |
| | ATAT | | T*+A*+T | | |
| 2509 | CTGTCTGAATGCCTC | A572507H | +C*+T*+G*T*C*T*G*A*A*T*G*C*C*T*C*+A* | 70453 | 18 |
| | ATA | | +T*+A | | |
| 2510 | GCTGTCTGAATGCCT | A572508H | +G*+C*+T*G*T*C*T*G*A*A*T*G*C*C*T*C*+ | 70453 | 19 |
| | CATA | | A*+T*+A | | |
| 2511 | AATGCTGTCTGAATG | A572509H | +A*+A*+T*G*C*T*G*T*C*T*G*A*A*T*G*C*+ | 70456 | 19 |
| | CCTC | | C*+T*+C | | |
| 2512 | TATTCTCTCTCAGAA | A572510H | +T*+A*+T*T*C*T*C*T*C*T*C*A*G*A*A*G*+ | 70482 | 19 |
| | GCCA | | C*+C*+A | | |
| 2513 | TGATGTAATATATTCT | A572511H | +T*+G*+A*T*G*T*A*A*T*A*T*A*T*T*C*T*+ | 70492 | 19 |
| | CTC | | C*+T*+C | | |
| 2514 | GTGATGTAATATATT | A572512H | +G*+T*+G*A*T*G*T*A*A*T*A*T*A*T*T*C*+ | 70493 | 19 |
| | CTCT | | T*+C*+T | | |
| 2515 | TAGTGATGTAATATA | A572513H | +T*+A*+G*T*G*A*T*G*T*A*A*T*A*T*A*T*+ | 70495 | 19 |
| | TTCT | | T*+C*+T | | |
| 2516 | GGTCTAGTGATGTAA | A572514H | +G*+G*+T*C*T*A*G*T*G*A*T*G*T*A*A*T*+ | 70499 | 19 |
| | TATA | | A*+T*+A | | |
| 2517 | AAGGTCTAGTGATGT | A572515H | +A*+A*+G*G*T*C*T*A*G*T*G*A*T*G*T*A*+ | 70501 | 19 |
| | AATA | | A*+T*+A | | |
| 2518 | AAAGGTCTAGTGATG | A572516H | +A*+A*+A*G*G*T*C*T*A*G*T*G*A*T*G*T*+ | 70502 | 19 |
| | TAAT | | A*+A*+T | | |
| 2519 | GAAAGGTCTAGTGAT | A572517H | +G*+A*+A*A*G*G*T*C*T*A*G*T*G*A*T*G*+ | 70503 | 19 |
| | GTAA | | T*+A*+A | | |
| 2520 | GAAAGGTCTAGTGAT | A572518H | +G*+A*+A*A*G*G*T*C*T*A*G*T*G*A*T*+G* | 70504 | 18 |
| | GTA | | +T*+A | | |
| 2521 | TGAAAGGTCTAGTGA | A572519H | +T*+G*+A*A*A*G*G*T*C*T*A*G*T*G*A*T*+ | 70504 | 19 |
| | TGTA | | G*+T*+A | | |
| 2522 | CTGAAAGGTCTAGTG | A572520H | +C*+T*+G*A*A*A*G*G*T*C*T*A*G*T*G*A*+ | 70505 | 19 |
| | ATGT | | T*+G*+T | | |
| 2523 | TGCTGAAAGGTCTAG | A572521H | +T*+G*+C*T*G*A*A*A*G*G*T*C*T*A*G*T*+ | 70507 | 19 |
| | TGAT | | G*+A*+T | | |
| 2524 | CATTTGCTGAAAGGT | A572522H | +C*+A*+T*T*T*G*C*T*G*A*A*A*G*G*T*C*+ | 70511 | 19 |
| | CTAG | | T*+A*+G | | |
| 2525 | AGTTCATTTGCTGAA | A572523H | +A*+G*+T*T*C*A*T*T*T*G*C*T*G*A*A*A*+ | 70515 | 19 |
| | AGGT | | G*+G*+T | | |
| 2526 | GCATCAATATCTCTT | A572524H | +G*+C*+A*T*C*A*A*T*A*T*C*T*C*T*T*A*+ | 70530 | 19 |
| | AGTT | | G*+T*+T | | |
| 2527 | GCATCAATATCTCTT | A572525H | +G*+C*+A*T*C*A*A*T*A*T*C*T*C*T*T*+A* | 70531 | 18 |
| | AGT | | +G*+T | | |
| 2528 | GGCATCAATATCTCT | A572526H | +G*+G*+C*A*T*C*A*A*T*A*T*C*T*C*T*T*+ | 70531 | 19 |
| | TAGT | | A*+G*+T | | |
| 2529 | GGGCATCAATATCTC | A572527H | +G*+G*+G*C*A*T*C*A*A*T*A*T*C*T*C*T*+ | 70532 | 19 |
| | TTAG | | T*+A*+G | | |
| 2530 | AGGGCATCAATATCT | A572528H | +A*+G*+G*G*C*A*T*C*A*A*T*A*T*C*T*C*+ | 70533 | 19 |
| | CTTA | | T*+T*+A | | |
| 2531 | TAGGGCATCAATATC | A572529H | +T*+A*+G*G*G*C*A*T*C*A*A*T*A*T*C*T*+ | 70534 | 19 |
| | TCTT | | C*+T*+T | | |
| 2532 | TTAGGGCATCAATAT | A572530H | +T*+T*+A*G*G*G*C*A*T*C*A*A*T*A*T*C*+ | 70535 | 19 |
| | CTCT | | T*+C*+T | | |
| 2533 | GCTTAGGGCATCAAT | A572531H | +G*+C*+T*T*A*G*G*G*C*A*T*C*A*A*T*A*+ | 70537 | 19 |
| | ATCT | | T*+C*+T | | |
| 2534 | GCTTAGGGCATCAAT | A572532H | +G*+C*+T*T*A*G*G*G*C*A*T*C*A*A*T*+A* | 70538 | 18 |
| | ATC | | +T*+C | | |
| 2535 | GGCTTAGGGCATCAA | A572533H | +G*+G*+C*T*T*A*G*G*G*C*A*T*C*A*A*T*+ | 70538 | 19 |
| | TATC | | A*+T*+C | | |
| 2536 | GGCTTAGGGCATCAA | A572534H | +G*+G*+C*T*T*A*G*G*G*C*A*T*C*A*A*+T* | 70539 | 18 |
| | TAT | | +A*+T | | |
| 2537 | TGGCTTAGGGCATCA | A572535H | +T*+G*+G*C*T*T*A*G*G*G*C*A*T*C*A*A*+ | 70539 | 19 |
| | ATAT | | T*+A*+T | | |
| 2538 | TGGCTTAGGGCATCA | A572536H | +T*+G*+G*C*T*T*A*G*G*G*C*A*T*C*A*+A* | 70540 | 18 |
| | ATA | | +T*+A | | |
| 2539 | CTGGCTTAGGGCATC | A572537H | +C*+T*+G*G*C*T*T*A*G*G*G*C*A*T*C*A*+ | 70540 | 19 |
| | AATA | | A*+T*+A | | |
| 2540 | CTGGCTTAGGGCATC | A572538H | +C*+T*+G*G*C*T*T*A*G*G*G*C*A*T*C*+A* | 70541 | 18 |
| | AAT | | +A*+T | | |
| 2541 | TCTGGCTTAGGGCAT | A572539H | +T*+C*+T*G*G*C*T*T*A*G*G*G*C*A*T*C*+ | 70541 | 19 |
| | CAAT | | A*+A*+T | | |
| 2542 | TTCTGGCTTAGGGCA | A572540H | +T*+T*+C*T*G*G*C*T*T*A*G*G*G*C*A*T*+ | 70542 | 19 |
| | TCAA | | C*+A*+A | | |
| 2543 | TTTCTGGCTTAGGGC | A572541H | +T*+T*+T*C*T*G*G*C*T*T*A*G*G*G*C*A*+ | 70543 | 19 |
| | ATCA | | T*+C*+A | | |
| 2544 | ATTTCTGGCTTAGGG | A572542H | +A*+T*+T*T*C*T*G*G*C*T*T*A*G*G*G*C*+ | 70544 | 19 |
| | CATC | | A*+T*+C | | |
| 2545 | TTTCAAGATGCTCCA | A572543H | +T*+T*+T*C*A*A*G*A*T*G*C*T*C*C*A*A*+ | 70577 | 19 |
| | AATG | | A*+T*+G | | |
| 2546 | GCTCCAGCTTTTCAA | A572544H | +G*+C*+T*C*C*A*G*C*T*T*T*T*C*A*A*G*+ | 70586 | 19 |
| | GATG | | A*+T*+G | | |
| 2547 | AGCTCCAGCTTTTCA | A572545H | +A*+G*+C*T*C*C*A*G*C*T*T*T*T*C*A*A*+ | 70587 | 19 |
| | AGAT | | G*+A*+T | | |
| 2548 | GTGAAGCTCCAGCTT | A572546H | +G*+T*+G*A*A*G*C*T*C*C*A*G*C*T*T*T*+ | 70591 | 19 |
| | TTCA | | T*+C*+A | | |
| 2549 | GGTGAAGCTCCAGCT | A572547H | +G*+G*+T*G*A*A*G*C*T*C*C*A*G*C*T*T*+ | 70592 | 19 |
| | TTTC | | T*+T*+C | | |
| 2550 | GGTGAAGCTCCAGCT | A572548H | +G*+G*+T*G*A*A*G*C*T*C*C*A*G*C*T*+T* | 70593 | 18 |
| | TTT | | +T*+T | | |
| 2551 | TGGTGAAGCTCCAGC | A572549H | +T*+G*+G*T*G*A*A*G*C*T*C*C*A*G*C*T*+ | 70593 | 19 |
| | TTTT | | T*+T*+T | | |
| 2552 | GCATTCTGGTGAAGC | A572550H | +G*+C*+A*T*T*C*T*G*G*T*G*A*A*G*C*T*+ | 70599 | 19 |
| | TCCA | | C*+C*+A | | |
| 2553 | GCATTCTGGTGAAGC | A572551H | +G*+C*+A*T*T*C*T*G*G*T*G*A*A*G*C*+T* | 70600 | 18 |
| | TCC | | +C*+C | | |
| 2554 | TGCATTCTGGTGAAG | A572552H | +T*+G*+C*A*T*T*C*T*G*G*T*G*A*A*G*C*+ | 70600 | 19 |
| | CTCC | | T*+C*+C | | |
| 2555 | TGCATTCTGGTGAAG | A572553H | +T*+G*+C*A*T*T*C*T*G*G*T*G*A*A*G*+C* | 70601 | 18 |
| | CTC | | +T*+C | | |
| 2556 | GTGCATTCTGGTGAA | A572554H | +G*+T*+G*C*A*T*T*C*T*G*G*T*G*A*A*G*+ | 70601 | 19 |
| | GCTC | | C*+T*+C | | |
| 2557 | AGTGCATTCTGGTGA | A572555H | +A*+G*+T*G*C*A*T*T*C*T*G*G*T*G*A*A*+ | 70602 | 19 |
| | AGCT | | G*+C*+T | | |
| 2558 | AGTGCATTCTGGTGA | A572556H | +A*+G*+T*G*C*A*T*T*C*T*G*G*T*G*A*+A* | 70603 | 18 |
| | AGC | | +G*+C | | |
| 2559 | GAGTGCATTCTGGTG | A572557H | +G*+A*+G*T*G*C*A*T*T*C*T*G*G*T*G*A*+ | 70603 | 19 |
| | AAGC | | A*+G*+C | | |
| 2560 | GAGTGCATTCTGGTG | A572558H | +G*+A*+G*T*G*C*A*T*T*C*T*G*G*T*G*+A* | 70604 | 18 |
| | AAG | | +A*+G | | |
| 2561 | TGAGTGCATTCTGGT | A572559H | +T*+G*+A*G*T*G*C*A*T*T*C*T*G*G*T*G*+ | 70604 | 19 |
| | GAAG | | A*+A*+G | | |
| 2562 | CGTGAGTGCATTCTG | A572560H | +C*+G*+T*G*A*G*T*G*C*A*T*T*C*T*G*G*+ | 70606 | 19 |
| | GTGA | | T*+G*+A | | |
| 2563 | CGTGAGTGCATTCTG | A572561H | +C*+G*+T*G*A*G*T*G*C*A*T*T*C*T*G*+G* | 70607 | 18 |
| | GTG | | +T*+G | | |
| 2564 | TCGTGAGTGCATTCT | A572562H | +T*+C*+G*T*G*A*G*T*G*C*A*T*T*C*T*G*+ | 70607 | 19 |
| | GGTG | | G*+T*+G | | |
| 2565 | TCGTGAGTGCATTCT | A572563H | +T*+C*+G*T*G*A*G*T*G*C*A*T*T*C*T*+G* | 70608 | 18 |
| | GGT | | +G*+T | | |
| 2566 | CTCGTGAGTGCATTC | A572564H | +C*+T*+C*G*T*G*A*G*T*G*C*A*T*T*C*T*+ | 70608 | 19 |
| | TGGT | | G*+G*+T | | |
| 2567 | TCGTGAGTGCATTCT | A572565H | +T*+C*+G*T*G*A*G*T*G*C*A*T*T*C*+T*+G | 70609 | 17 |
| | GG | | *+G | | |
| 2568 | CTCGTGAGTGCATTC | A572566H | +C*+T*+C*G*T*G*A*G*T*G*C*A*T*T*C*+T* | 70609 | 18 |
| | TGG | | +G*+G | | |
| 2569 | GCTCGTGAGTGCATT | A572567H | +G*+C*+T*C*G*T*G*A*G*T*G*C*A*T*T*C*+ | 70609 | 19 |
| | CTGG | | T*+G*+G | | |
| 2570 | GCTCGTGAGTGCATT | A572568H | +G*+C*+T*C*G*T*G*A*G*T*G*C*A*T*T*+C* | 70610 | 18 |
| | CTG | | +T*+G | | |
| 2571 | AGCTCGTGAGTGCAT | A572569H | +A*+G*+C*T*C*G*T*G*A*G*T*G*C*A*T*T*+ | 70610 | 19 |
| | TCTG | | C*+T*+G | | |
| 2572 | AGCTCGTGAGTGCAT | A572570H | +A*+G*+C*T*C*G*T*G*A*G*T*G*C*A*T*+T* | 70611 | 18 |
| | TCT | | +C*+T | | |
| 2573 | AAGCTCGTGAGTGCA | A572571H | +A*+A*+G*C*T*C*G*T*G*A*G*T*G*C*A*T*+ | 70611 | 19 |
| | TTCT | | T*+C*+T | | |
| 2574 | AAGCTCGTGAGTGCA | A572572H | +A*+A*+G*C*T*C*G*T*G*A*G*T*G*C*A*+T* | 70612 | 18 |
| | TTC | | +T*+C | | |
| 2575 | AAAGCTCGTGAGTGC | A572573H | +A*+A*+A*G*C*T*C*G*T*G*A*G*T*G*C*A*+ | 70612 | 19 |
| | ATTC | | T*+T*+C | | |
| 2576 | GAAAGCTCGTGAGTG | A572574H | +G*+A*+A*A*G*C*T*C*G*T*G*A*G*T*G*C*+ | 70613 | 19 |
| | CATT | | A*+T*+T | | |
| 2577 | GAAAGCTCGTGAGTG | A572575H | +G*+A*+A*A*G*C*T*C*G*T*G*A*G*T*G*+C* | 70614 | 18 |
| | CAT | | +A*+T | | |
| 2578 | GGAAAGCTCGTGAGT | A572576H | +G*+G*+A*A*A*G*C*T*C*G*T*G*A*G*T*G* | 70614 | 19 |
| | GCAT | | +C*+A*+T | | |
| 2579 | GTGGAAAGCTCGTGA | A572577H | +G*+T*+G*G*A*A*A*G*C*T*C*G*T*G*A*G* | 70616 | 19 |
| | GTGC | | +T*+G*+C | | |
| 2580 | TGTGGAAAGCTCGTG | A572578H | +T*+G*+T*G*G*A*A*A*G*C*T*C*G*T*G*A*+ | 70617 | 19 |
| | AGTG | | G*+T*+G | | |
| 2581 | TGTGGAAAGCTCGTG | A572579H | +T*+G*+T*G*G*A*A*A*G*C*T*C*G*T*G*+A* | 70618 | 18 |
| | AGT | | +G*+T | | |
| 2582 | TTGTGGAAAGCTCGT | A572580H | +T*+T*+G*T*G*G*A*A*A*G*C*T*C*G*T*G*+ | 70618 | 19 |
| | GAGT | | A*+G*+T | | |
| 2583 | TTGTGGAAAGCTCGT | A572581H | +T*+T*+G*T*G*G*A*A*A*G*C*T*C*G*T*+G* | 70619 | 18 |
| | GAG | | +A*+G | | |
| 2584 | GTTGTGGAAAGCTCG | A572582H | +G*+T*+T*G*T*G*G*A*A*A*G*C*T*C*G*T*+ | 70619 | 19 |
| | TGAG | | G*+A*+G | | |
| 2585 | GTTGTGGAAAGCTCG | A572583H | +G*+T*+T*G*T*G*G*A*A*A*G*C*T*C*G*+T* | 70620 | 18 |
| | TGA | | +G*+A | | |
| 2586 | TGTTGTGGAAAGCTC | A572584H | +T*+G*+T*T*G*T*G*G*A*A*A*G*C*T*C*G*+ | 70620 | 19 |
| | GTGA | | T*+G*+A | | |
| 2587 | TGTTGTGGAAAGCTC | A572585H | +T*+G*+T*T*G*T*G*G*A*A*A*G*C*T*C*+G* | 70621 | 18 |
| | GTG | | +T*+G | | |
| 2588 | CTGTTGTGGAAAGCT | A572586H | +C*+T*+G*T*T*G*T*G*G*A*A*A*G*C*T*C*+ | 70621 | 19 |
| | CGTG | | G*+T*+G | | |
| 2589 | TGTTGTGGAAAGCTC | A572587H | +T*+G*+T*T*G*T*G*G*A*A*A*G*C*T*+C*+G | 70622 | 17 |
| | GT | | *+T | | |
| 2590 | CTGTTGTGGAAAGCT | A572588H | +C*+T*+G*T*T*G*T*G*G*A*A*A*G*C*T*+C* | 70622 | 18 |
| | CGT | | +G*+T | | |
| 2591 | GCTGTTGTGGAAAGC | A572589H | +G*+C*+T*G*T*T*G*T*G*G*A*A*A*G*C*T*+ | 70622 | 19 |
| | TCGT | | C*+G*+T | | |
| 2592 | CTGTTGTGGAAAGCT | A572590H | +C*+T*+G*T*T*G*T*G*G*A*A*A*G*C*+T*+C | 70623 | 17 |
| | CG | | *+G | | |
| 2593 | GCTGTTGTGGAAAGC | A572591H | +G*+C*+T*G*T*T*G*T*G*G*A*A*A*G*C*+T* | 70623 | 18 |
| | TCG | | +C*+G | | |
| 2594 | AGCTGTTGTGGAAAG | A572592H | +A*+G*+C*T*G*T*T*G*T*G*G*A*A*A*G*C*+ | 70623 | 19 |
| | CTCG | | T*+C*+G | | |
| 2595 | TAGCTGTTGTGGAAA | A572593H | +T*+A*+G*C*T*G*T*T*G*T*G*G*A*A*A*G*+ | 70624 | 19 |
| | GCTC | | C*+T*+C | | |
| 2596 | ATAGCTGTTGTGGAA | A572594H | +A*+T*+A*G*C*T*G*T*T*G*T*G*G*A*A*A*+ | 70625 | 19 |
| | AGCT | | G*+C*+T | | |
| 2597 | ACATAGCTGTTGTGG | A572595H | +A*+C*+A*T*A*G*C*T*G*T*T*G*T*G*G*A*+ | 70627 | 19 |
| | AAAG | | A*+A*+G | | |
| 2598 | CACATAGCTGTTGTG | A572596H | +C*+A*+C*A*T*A*G*C*T*G*T*T*G*T*G*G*+ | 70628 | 19 |
| | GAAA | | A*+A*+A | | |
| 2599 | AGACTAAGATATGTG | A572597Hi | +A*+G*+A*C*T*A*A*G*A*T*A*T*G*T*G*T*+ | 70788 | 19 |
| | TGCA | | G*+C*+A | | |
| 2600 | ACACAGACTAAGATA | A572598Hi | +A*+C*+A*C*A*G*A*C*T*A*A*G*A*T*A*T*+ | 70792 | 19 |
| | TGTG | | G*+T*+G | | |
| 2601 | CTTTCTAATCAGATCT | A572599Hi | +C*+T*+T*T*C*T*A*A*T*C*A*G*A*T*C*T*+ | 70846 | 19 |
| | GTT | | G*+T*+T | | |
| 2602 | TTCTAGTTTACCTAAT | A572600Hi | +T*+T*+C*T*A*G*T*T*T*A*C*C*T*A*A*T*+ | 70967 | 19 |
| | CTA | | C*+T*+A | | |
| 2603 | AATTCTAGTTTACCTA | A572601Hi | +A*+A*+T*T*C*T*A*G*T*T*T*A*C*C*T*A*+ | 70969 | 19 |
| | ATC | | A*+T*+C | | |
| 2604 | ACCAAACCAGGTCTA | A572602Hi | +A*+C*+C*A*A*A*C*C*A*G*G*T*C*T*A*A*+ | 70983 | 19 |
| | ATTC | | T*+T*+C | | |
| 2605 | CTACCAAACCAGGTC | A572603Hi | +C*+T*+A*C*C*A*A*A*C*C*A*G*G*T*C*T*+ | 70985 | 19 |
| | TAAT | | A*+A*+T | | |
| 2606 | TACCAAACCAGGTCT | A572604Hi | +T*+A*+C*C*A*A*A*C*C*A*G*G*T*C*T*+A* | 70985 | 18 |
| | AAT | | +A*+T | | |
| 2607 | ACTACCAAACCAGGT | A572605Hi | +A*+C*+T*A*C*C*A*A*A*C*C*A*G*G*T*C*+ | 70986 | 19 |
| | CTAA | | T*+A*+A | | |
| 2608 | ACTACCAAACCAGGT | A572606Hi | +A*+C*+T*A*C*C*A*A*A*C*C*A*G*G*T*+C* | 70987 | 18 |
| | CTA | | +T*+A | | |
| 2609 | GCCAGTACTACCAAA | A572607Hi | +G*+C*+C*A*G*T*A*C*T*A*C*C*A*A*A*+C* | 70993 | 18 |
| | CCA | | +C*+A | | |
| 2610 | GAGCCAGTACTACCA | A572608Hi | +G*+A*+G*C*C*A*G*T*A*C*T*A*C*C*A*A*+ | 70994 | 19 |
| | AACC | | A*+C*+C | | |
| 2611 | GAGCCAGTACTACCA | A572609Hi | +G*+A*+G*C*C*A*G*T*A*C*T*A*C*C*A*+A* | 70995 | 18 |
| | AAC | | +A*+C | | |
| 2612 | AGAGCCAGTACTACC | A572610Hi | +A*+G*+A*G*C*C*A*G*T*A*C*T*A*C*C*A*+ | 70995 | 19 |
| | AAAC | | A*+A*+C | | |
| 2613 | TAGCTGAGTCAGAGC | A572611Hi | +T*+A*+G*C*T*G*A*G*T*C*A*G*A*G*C*C*+ | 71005 | 19 |
| | CAGT | | A*+G*+T | | |
| 2614 | TCATTTAGTTGAATCT | A572612Hi | +T*+C*+A*T*T*T*A*G*T*T*G*A*A*T*C*T*+ | 71091 | 19 |
| | CTA | | C*+T*+A | | |
| 2615 | CTATCCTATCAACAA | A572613Hi | +C*+T*+A*T*C*C*T*A*T*C*A*A*C*A*A*A*+ | 71208 | 19 |
| | ACTA | | C*+T*+A | | |
| 2616 | GCTATCCTATCAACA | A572614Hi | +G*+C*+T*A*T*C*C*T*A*T*C*A*A*C*A*A*+ | 71209 | 19 |
| | AACT | | A*+C*+T | | |
| 2617 | CTATCCTATCAACAA | A572615Hi | +C*+T*+A*T*C*C*T*A*T*C*A*A*C*A*A*+A* | 71209 | 18 |
| | ACT | | +C*+T | | |
| 2618 | AGCTATCCTATCAAC | A572616Hi | +A*+G*+C*T*A*T*C*C*T*A*T*C*A*A*C*A*+ | 71210 | 19 |
| | AAAC | | A*+A*+C | | |
| 2619 | GCTATCCTATCAACA | A572617Hi | +G*+C*+T*A*T*C*C*T*A*T*C*A*A*C*A*+A* | 71210 | 18 |
| | AAC | | +A*+C | | |
| 2620 | AGAGCTATCCTATCA | A572618Hi | +A*+G*+A*G*C*T*A*T*C*C*T*A*T*C*A*A*+ | 71212 | 19 |
| | ACAA | | C*+A*+A | | |
| 2621 | AGAGCTATCCTATCA | A572619Hi | +A*+G*+A*G*C*T*A*T*C*C*T*A*T*C*A*+A* | 71213 | 18 |
| | ACA | | +C*+A | | |
| 2622 | TTCAGAGAGCTATCC | A572620Hi | +T*+T*+C*A*G*A*G*A*G*C*T*A*T*C*C*+T* | 71218 | 18 |
| | TAT | | +A*+T | | |
| 2623 | ATATCTTCTTTGGCTT | A572621Hi | +A*+T*+A*T*C*T*T*C*T*T*T*G*G*C*T*T*+ | 71283 | 19 |
| | TCA | | T*+C*+A | | |
| 2624 | GCATTTGCAACAAGA | A572622Hi | +G*+C*+A*T*T*T*G*C*A*A*C*A*A*G*A*C*+ | 71645 | 19 |
| | CTTT | | T*+T*+T | | |
| 2625 | GCATTTGCAACAAGA | A572623Hi | +G*+C*+A*T*T*T*G*C*A*A*C*A*A*G*A*+C* | 71646 | 18 |
| | CTT | | +T*+T | | |
| 2626 | GAAATGCATTTGCAA | A572624Hi | +G*+A*+A*A*T*G*C*A*T*T*T*G*C*A*A*C*+ | 71650 | 19 |
| | CAAG | | A*+A*+G | | |
| 2627 | AAGTATTTAGCAAGG | A572625Hi | +A*+A*+G*T*A*T*T*T*A*G*C*A*A*G*G*C*+ | 71865 | 19 |
| | CAAA | | A*+A*+A | | |
| 2628 | GAAGTATTTAGCAAG | A572626Hi | +G*+A*+A*G*T*A*T*T*T*A*G*C*A*A*G*G*+ | 71866 | 19 |
| | GCAA | | C*+A*+A | | |
| 2629 | GAAGTATTTAGCAAG | A572627Hi | +G*+A*+A*G*T*A*T*T*T*A*G*C*A*A*G*+G* | 71867 | 18 |
| | GCA | | +C*+A | | |
| 2630 | TGAAGTATTTAGCAA | A572628Hi | +T*+G*+A*A*G*T*A*T*T*T*A*G*C*A*A*+G* | 71868 | 18 |
| | GGC | | +G*+C | | |
| 2631 | GCAGAGAGTGATGAA | A572629Hi | +G*+C*+A*G*A*G*A*G*T*G*A*T*G*A*A*G* | 71911 | 19 |
| | GTGA | | +T*+G*+A | | |
| 2632 | TTAAATCAAATTGGA | A572630Hi | +T*+T*+A*A*A*T*C*A*A*A*T*T*G*G*A*T*+ | 72022 | 19 |
| | TGAC | | G*+A*+C | | |
| 2633 | AATAGACAGAAACAA | A572631Hi | +A*+A*+T*A*G*A*C*A*G*A*A*A*C*A*A*G*+ | 72093 | 19 |
| | GCCT | | C*+C*+T | | |
| 2634 | GGAATAGACAGAAAC | A572632Hi | +G*+G*+A*A*T*A*G*A*C*A*G*A*A*A*C*A*+ | 72095 | 19 |
| | AAGC | | A*+G*+C | | |
| 2635 | CCTCCACAAATCAAT | A572633Hi | +C*+C*+T*C*C*A*C*A*A*A*T*C*A*A*T*T*+ | 72158 | 19 |
| | TCAG | | C*+A*+G | | |
| 2636 | CTCCACAAATCAATT | A572634Hi | +C*+T*+C*C*A*C*A*A*A*T*C*A*A*T*T*+C* | 72158 | 18 |
| | CAG | | +A*+G | | |
| 2637 | GTCCAGTCATTGAAT | A572635Hi | +G*+T*+C*C*A*G*T*C*A*T*T*G*A*A*T*+G* | 72304 | 18 |
| | GCC | | +C*+C | | |
| 2638 | TGTCCAGTCATTGAA | A572636Hi | +T*+G*+T*C*C*A*G*T*C*A*T*T*G*A*A*+T* | 72305 | 18 |
| | TGC | | +G*+C | | |
| 2639 | AGCTTGTCCAGTCAT | A572637Hi | +A*+G*+C*T*T*G*T*C*C*A*G*T*C*A*T*+T* | 72309 | 18 |
| | TGA | | +G*+A | | |
| 2640 | GGTTCTGAGCTTGTC | A572638Hi | +G*+G*+T*T*C*T*G*A*G*C*T*T*G*T*C*+C* | 72316 | 18 |
| | CAG | | +A*+G | | |
| 2641 | AAGGTTCTGAGCTTG | A572639Hi | +A*+A*+G*G*T*T*C*T*G*A*G*C*T*T*G*+T* | 72318 | 18 |
| | TCC | | +C*+C | | |
| 2642 | GAAGGTTCTGAGCTT | A572640Hi | +G*+A*+A*G*G*T*T*C*T*G*A*G*C*T*T*+G* | 72319 | 18 |
| | GTC | | +T*+C | | |
| 2643 | AGAGAAGGTTCTGAG | A572641Hi | +A*+G*+A*G*A*A*G*G*T*T*C*T*G*A*G*C*+ | 72321 | 19 |
| | CTTG | | T*+T*+G | | |
| 2644 | GAGAAGGTTCTGAGC | A572642Hi | +G*+A*+G*A*A*G*G*T*T*C*T*G*A*G*C*+T* | 72321 | 18 |
| | TTG | | +T*+G | | |
| 2645 | AAGAGAAGGTTCTGA | A572643Hi | +A*+A*+G*A*G*A*A*G*G*T*T*C*T*G*A*G*+ | 72322 | 19 |
| | GCTT | | C*+T*+T | | |
| 2646 | CCATCTAATGGTGTC | A572644Hi | +C*+C*+A*T*C*T*A*A*T*G*G*T*G*T*C*A*+ | 72353 | 19 |
| | ATTT | | T*+T*+T | | |
| 2647 | GTTGAAAGCTTCCTT | A572645Hi | +G*+T*+T*G*A*A*A*G*C*T*T*C*C*T*T*C*+ | 72988 | 19 |
| | CAGC | | A*+G*+C | | |
| 2648 | AGTTGAAAGCTTCCT | A572646Hi | +A*+G*+T*T*G*A*A*A*G*C*T*T*C*C*T*T*+ | 72989 | 19 |
| | TCAG | | C*+A*+G | | |
| 2649 | GAGTTGAAAGCTTCC | A572647Hi | +G*+A*+G*T*T*G*A*A*A*G*C*T*T*C*C*T*+ | 72990 | 19 |
| | TTCA | | T*+C*+A | | |
| 2650 | AGAGTTGAAAGCTTC | A572648Hi | +A*+G*+A*G*T*T*G*A*A*A*G*C*T*T*C*C*+ | 72991 | 19 |
| | CTTC | | T*+T*+C | | |
| 2651 | TAGAGTTGAAAGCTT | A572649Hi | +T*+A*+G*A*G*T*T*G*A*A*A*G*C*T*T*C*+ | 72992 | 19 |
| | CCTT | | C*+T*+T | | |
| 2652 | ATAGAGTTGAAAGCT | A572650Hi | +A*+T*+A*G*A*G*T*T*G*A*A*A*G*C*T*+T* | 72994 | 18 |
| | TCC | | +C*+C | | |
| 2653 | AATAGAGTTGAAAGC | A572651Hi | +A*+A*+T*A*G*A*G*T*T*G*A*A*A*G*C*T*+ | 72994 | 19 |
| | TTCC | | T*+C*+C | | |
| 2654 | CAACAGAATTTCAGT | A572652Hi | +C*+A*+A*C*A*G*A*A*T*T*T*C*A*G*T*A*+ | 73097 | 19 |
| | ATAC | | T*+A*+C | | |
| 2655 | AACTAGAACCATCAA | A572653Hi | +A*+A*+C*T*A*G*A*A*C*C*A*T*C*A*A*G*+ | 73142 | 19 |
| | GAAT | | A*+A*+T | | |
| 2656 | ACTAGAACCATCAAG | A572654Hi | +A*+C*+T*A*G*A*A*C*C*A*T*C*A*A*G*+A* | 73142 | 18 |
| | AAT | | +A*+T | | |
| 2657 | CTGGTAACTAGAACC | A572655Hi | +C*+T*+G*G*T*A*A*C*T*A*G*A*A*C*C*A*+ | 73147 | 19 |
| | ATCA | | T*+C*+A | | |
| 2658 | TCTGGTAACTAGAAC | A572656Hi | +T*+C*+T*G*G*T*A*A*C*T*A*G*A*A*C*C*+ | 73148 | 19 |
| | CATC | | A*+T*+C | | |
| 2659 | CTGGTAACTAGAACC | A572657Hi | +C*+T*+G*G*T*A*A*C*T*A*G*A*A*C*C*+A* | 73148 | 18 |
| | ATC | | +T*+C | | |
| 2660 | CACCTCTGGTAACTA | A572658Hi | +C*+A*+C*C*T*C*T*G*G*T*A*A*C*T*A*+G* | 73153 | 18 |
| | GAA | | +A*+A | | |
| 2661 | GCCTTACACACCTCT | A572659Hi | +G*+C*+C*T*T*A*C*A*C*A*C*C*T*C*T*G*+ | 73160 | 19 |
| | GGTA | | G*+T*+A | | |
| 2662 | CCTTACACACCTCTG | A572660Hi | +C*+C*+T*T*A*C*A*C*A*C*C*T*C*T*G*+G* | 73160 | 18 |
| | GTA | | +T*+A | | |
| 2663 | GCCTTACACACCTCT | A572661Hi | +G*+C*+C*T*T*A*C*A*C*A*C*C*T*C*T*+G* | 73161 | 18 |
| | GGT | | +G*+T | | |
| 2664 | TAATATTTCTGCCTTA | A572662Hi | +T*+A*+A*T*A*T*T*T*C*T*G*C*C*T*T*A*+ | 73170 | 19 |
| | CAC | | C*+A*+C | | |
| 2665 | GTCTAGCTAATATTT | A572663Hi | +G*+T*+C*T*A*G*C*T*A*A*T*A*T*T*T*C*+ | 73177 | 19 |
| | CTGC | | T*+G*+C | | |
| 2666 | AGTCTAGCTAATATT | A572664Hi | +A*+G*+T*C*T*A*G*C*T*A*A*T*A*T*T*+T* | 73179 | 18 |
| | TCT | | +C*+T | | |
| 2667 | GAGGAACTTAAGTCT | A572665Hi | +G*+A*+G*G*A*A*C*T*T*A*A*G*T*C*T*+A* | 73189 | 18 |
| | AGC | | +G*+C | | |
| 2668 | GTCCAAATGTGTCAA | A572666H | +G*+T*+C*C*A*A*A*T*G*T*G*T*C*A*A*A*+ | 73255 | 19 |
| | ACTC | | C*+T*+C | | |
| 2669 | AGTCCAAATGTGTCA | A572667H | +A*+G*+T*C*C*A*A*A*T*G*T*G*T*C*A*A*+ | 73256 | 19 |
| | AACT | | A*+C*+T | | |
| 2670 | AAGTCCAAATGTGTC | A572668H | +A*+A*+G*T*C*C*A*A*A*T*G*T*G*T*C*A*+ | 73257 | 19 |
| | AAAC | | A*+A*+C | | |
| 2671 | CTGTGCAAGTCCAAA | A572669H | +C*+T*+G*T*G*C*A*A*G*T*C*C*A*A*A*T*+ | 73263 | 19 |
| | TGTG | | G*+T*+G | | |
| 2672 | TATTACTGTGCAAGT | A572670H | +T*+A*+T*T*A*C*T*G*T*G*C*A*A*G*T*C*+ | 73268 | 19 |
| | CCAA | | C*+A*+A | | |
| 2673 | ATTTATTACTGTGCA | A572671H | +A*+T*+T*T*A*T*T*A*C*T*G*T*G*C*A*A*+ | 73271 | 19 |
| | AGTC | | G*+T*+C | | |
| 2674 | AATTTATTACTGTGC | A572672H | +A*+A*+T*T*T*A*T*T*A*C*T*G*T*G*C*A*+ | 73272 | 19 |
| | AAGT | | A*+G*+T | | |
| 2675 | ATGTAAATTTATTACT | A572673H | +A*+T*+G*T*A*A*A*T*T*T*A*T*T*A*C*T*+ | 73277 | 19 |
| | GTG | | G*+T*+G | | |
| 2676 | TGATGTAAATTTATTA | A572674H | +T*+G*+A*T*G*T*A*A*A*T*T*T*A*T*T*A*+ | 73279 | 19 |
| | CTG | | C*+T*+G | | |
| 2677 | AATGATGTAAATTTA | A572675H | +A*+A*+T*G*A*T*G*T*A*A*A*T*T*T*A*T*+ | 73281 | 19 |
| | TTAC | | T*+A*+C | | |
| 2678 | CATTTCGAGAGACAT | A572676H | +C*+A*+T*T*T*C*G*A*G*A*G*A*C*A*T*C*+ | 73337 | 19 |
| | CAAG | | A*+A*+G | | |
| 2679 | CATTTCGAGAGACAT | A572677H | +C*+A*+T*T*T*C*G*A*G*A*G*A*C*A*T*+C* | 73338 | 18 |
| | CAA | | +A*+A | | |
| 2680 | TCATTTCGAGAGACA | A572678H | +T*+C*+A*T*T*T*C*G*A*G*A*G*A*C*A*T*+ | 73338 | 19 |
| | TCAA | | C*+A*+A | | |
| 2681 | GTCATTTCGAGAGAC | A572679H | +G*+T*+C*A*T*T*T*C*G*A*G*A*G*A*C*A*+ | 73339 | 19 |
| | ATCA | | T*+C*+A | | |
| 2682 | AATGTCATTTCGAGA | A572680H | +A*+A*+T*G*T*C*A*T*T*T*C*G*A*G*A*G*+ | 73342 | 19 |
| | GACA | | A*+C*+A | | |
| 2683 | AATGTCATTTCGAGA | A572681H | +A*+A*+T*G*T*C*A*T*T*T*C*G*A*G*A*+G* | 73343 | 18 |
| | GAC | | +A*+C | | |
| 2684 | CAATGTCATTTCGAG | A572682H | +C*+A*+A*T*G*T*C*A*T*T*T*C*G*A*G*A*+ | 73343 | 19 |
| | AGAC | | G*+A*+C | | |
| 2685 | TCCAATGTCATTTCG | A572683H | +T*+C*+C*A*A*T*G*T*C*A*T*T*T*C*G*A*+ | 73345 | 19 |
| | AGAG | | G*+A*+G | | |
| 2686 | TCCAATGTCATTTCG | A572684H | +T*+C*+C*A*A*T*G*T*C*A*T*T*T*C*G*+A* | 73346 | 18 |
| | AGA | | +G*+A | | |
| 2687 | GTCCAATGTCATTTC | A572685H | +G*+T*+C*C*A*A*T*G*T*C*A*T*T*T*C*G*+ | 73346 | 19 |
| | GAGA | | A*+G*+A | | |
| 2688 | GTCCAATGTCATTTC | A572686H | +G*+T*+C*C*A*A*T*G*T*C*A*T*T*T*C*+G* | 73347 | 18 |
| | GAG | | +A*+G | | |
| 2689 | GGTCCAATGTCATTT | A572687H | +G*+G*+T*C*C*A*A*T*G*T*C*A*T*T*T*C*+ | 73347 | 19 |
| | CGAG | | G*+A*+G | | |
| 2690 | GGTCCAATGTCATTT | A572688H | +G*+G*+T*C*C*A*A*T*G*T*C*A*T*T*T*+C* | 73348 | 18 |
| | CGA | | +G*+A | | |
| 2691 | GGGTCCAATGTCATT | A572689H | +G*+G*+G*T*C*C*A*A*T*G*T*C*A*T*T*T*+ | 73348 | 19 |
| | TCGA | | C*+G*+A | | |
| 2692 | AGGGTCCAATGTCAT | A572690H | +A*+G*+G*G*T*C*C*A*A*T*G*T*C*A*T*T*+ | 73349 | 19 |
| | TTCG | | T*+C*+G | | |
| 2693 | ACTGAGGGTCCAATG | A572691H | +A*+C*+T*G*A*G*G*G*T*C*C*A*A*T*G*T*+ | 73353 | 19 |
| | TCAT | | C*+A*+T | | |
| 2694 | CACTGAGGGTCCAAT | A572692H | +C*+A*+C*T*G*A*G*G*G*T*C*C*A*A*T*G*+ | 73354 | 19 |
| | GTCA | | T*+C*+A | | |
| 2695 | ACCACTGAGGGTCCA | A572693H | +A*+C*+C*A*C*T*G*A*G*G*G*T*C*C*A*A*+ | 73356 | 19 |
| | ATGT | | T*+G*+T | | |
| 2696 | AACCACTGAGGGTCC | A572694H | +A*+A*+C*C*A*C*T*G*A*G*G*G*T*C*C*A*+ | 73357 | 19 |
| | AATG | | A*+T*+G | | |
| 2697 | AACCACTGAGGGTCC | A572695H | +A*+A*+C*C*A*C*T*G*A*G*G*G*T*C*C*+A* | 73358 | 18 |
| | AAT | | +A*+T | | |
| 2698 | AAACCACTGAGGGTC | A572696H | +A*+A*+A*C*C*A*C*T*G*A*G*G*G*T*C*C*+ | 73358 | 19 |
| | CAAT | | A*+A*+T | | |
| 2699 | ATCTAAAACCACTGA | A572697H | +A*+T*+C*T*A*A*A*A*C*C*A*C*T*G*A*G*+ | 73363 | 19 |
| | GGGT | | G*+G*+T | | |
| 2700 | GGATCTAAAACCACT | A572698H | +G*+G*+A*T*C*T*A*A*A*A*C*C*A*C*T*G*+ | 73365 | 19 |
| | GAGG | | A*+G*+G | | |
| 2701 | TCAGAGTTGGACATT | A572699H | +T*+C*+A*G*A*G*T*T*G*G*A*C*A*T*T*T*+ | 73388 | 19 |
| | TCAC | | C*+A*+C | | |
| 2702 | CTGTTTCAGAGTTGG | A572700H | +C*+T*+G*T*T*T*C*A*G*A*G*T*T*G*G*A*+ | 73393 | 19 |
| | ACAT | | C*+A*+T | | |
| 2703 | ACTGTTTCAGAGTTG | A572701H | +A*+C*+T*G*T*T*T*C*A*G*A*G*T*T*G*G*+ | 73394 | 19 |
| | GACA | | A*+C*+A | | |
| 2704 | AACTGTTTCAGAGTT | A572702H | +A*+A*+C*T*G*T*T*T*C*A*G*A*G*T*T*G*+ | 73395 | 19 |
| | GGAC | | G*+A*+C | | |
| 2705 | TAAACTGTTTCAGAG | A572703H | +T*+A*+A*A*C*T*G*T*T*T*C*A*G*A*G*T*+ | 73397 | 19 |
| | TTGG | | T*+G*+G | | |
| 2706 | GGTTAAACTGTTTCA | A572704H | +G*+G*+T*T*A*A*A*C*T*G*T*T*T*C*A*G*+ | 73400 | 19 |
| | GAGT | | A*+G*+T | | |
| 2707 | TGACAGGTTAAACTG | A572705H | +T*+G*+A*C*A*G*G*T*T*A*A*A*C*T*G*T*+ | 73405 | 19 |
| | TTTC | | T*+T*+C | | |
| 2708 | TATGACAGGTTAAAC | A572706H | +T*+A*+T*G*A*C*A*G*G*T*T*A*A*A*C*T*+ | 73407 | 19 |
| | TGTT | | G*+T*+T | | |
| 2709 | ATATGACAGGTTAAA | A572707H | +A*+T*+A*T*G*A*C*A*G*G*T*T*A*A*A*C*+ | 73408 | 19 |
| | CTGT | | T*+G*+T | | |
| 2710 | TATATGACAGGTTAA | A572708H | +T*+A*+T*A*T*G*A*C*A*G*G*T*T*A*A*A*+ | 73409 | 19 |
| | ACTG | | C*+T*+G | | |
| 2711 | TTATATGACAGGTTA | A572709H | +T*+T*+A*T*A*T*G*A*C*A*G*G*T*T*A*A*+ | 73410 | 19 |
| | AACT | | A*+C*+T | | |
| 2712 | GTTATATGACAGGTT | A572710H | +G*+T*+T*A*T*A*T*G*A*C*A*G*G*T*T*A*+ | 73411 | 19 |
| | AAAC | | A*+A*+C | | |
| 2713 | CTGGTTATATGACAG | A572711H | +C*+T*+G*G*T*T*A*T*A*T*G*A*C*A*G*G*+ | 73414 | 19 |
| | GTTA | | T*+T*+A | | |
| 2714 | CTGGTTATATGACAG | A572712H | +C*+T*+G*G*T*T*A*T*A*T*G*A*C*A*G*+G* | 73415 | 18 |
| | GTT | | +T*+T | | |
| 2715 | GCTGGTTATATGACA | A572713H | +G*+C*+T*G*G*T*T*A*T*A*T*G*A*C*A*G*+ | 73415 | 19 |
| | GGTT | | G*+T*+T | | |
| 2716 | GCTGGTTATATGACA | A572714H | +G*+C*+T*G*G*T*T*A*T*A*T*G*A*C*A*+G* | 73416 | 18 |
| | GGT | | +G*+T | | |
| 2717 | AGCTGGTTATATGAC | A572715H | +A*+G*+C*T*G*G*T*T*A*T*A*T*G*A*C*A*+ | 73416 | 19 |
| | AGGT | | G*+G*+T | | |
| 2718 | CTAAAATGAGCTGCT | A572716H | +C*+T*+A*A*A*A*T*G*A*G*C*T*G*C*T*C*+ | 73475 | 19 |
| | CCAG | | C*+A*+G | | |
| 2719 | TTCTAAAATGAGCTG | A572717H | +T*+T*+C*T*A*A*A*A*T*G*A*G*C*T*G*C*+ | 73477 | 19 |
| | CTCC | | T*+C*+C | | |
| 2720 | CTTCTAAAATGAGCT | A572718H | +C*+T*+T*C*T*A*A*A*A*T*G*A*G*C*T*G*+ | 73478 | 19 |
| | GCTC | | C*+T*+C | | |
| 2721 | CATCAGCATATTTAG | A572719Hi | +C*+A*+T*C*A*G*C*A*T*A*T*T*T*A*G*+G* | 73529 | 18 |
| | GCA | | +C*+A | | |
| 2722 | GAGGACTCATTAATT | A572720Hi | +G*+A*+G*G*A*C*T*C*A*T*T*A*A*T*T*T*+ | 73996 | 19 |
| | TAAA | | A*+A*+A | | |
| 2723 | AAGAGGACTCATTAA | A572721Hi | +A*+A*+G*A*G*G*A*C*T*C*A*T*T*A*A*T*+ | 73998 | 19 |
| | TTTA | | T*+T*+A | | |
| 2724 | ACAAGAGACAAGATA | A572722Hi | +A*+C*+A*A*G*A*G*A*C*A*A*G*A*T*A*A*+ | 74088 | 19 |
| | ATGA | | T*+G*+A | | |
| 2725 | CACAAGAGACAAGAT | A572723Hi | +C*+A*+C*A*A*G*A*G*A*C*A*A*G*A*T*A*+ | 74089 | 19 |
| | AATG | | A*+T*+G | | |
| 2726 | GAGCATATCCCTGAT | A572724H | +G*+A*+G*C*A*T*A*T*C*C*C*T*G*A*T*A*+ | 74118 | 19 |
| | ATTT | | T*+T*+T | | |
| 2727 | GGAGCATATCCCTGA | A572725H | +G*+G*+A*G*C*A*T*A*T*C*C*C*T*G*A*T*+ | 74119 | 19 |
| | TATT | | A*+T*+T | | |
| 2728 | AAGGGGGAGCATATC | A572726H | +A*+A*+G*G*G*G*G*A*G*C*A*T*A*T*C*C* | 74124 | 19 |
| | CCTG | | +C*+T*+G | | |
| 2729 | AAGGGGGAGCATATC | A572727H | +A*+A*+G*G*G*G*G*A*G*C*A*T*A*T*C*+C | 74125 | 18 |
| | CCT | | *+C*+T | | |
| 2730 | CAAGGGGGAGCATAT | A572728H | +C*+A*+A*G*G*G*G*G*A*G*C*A*T*A*T*C* | 74125 | 19 |
| | CCCT | | +C*+C*+T | | |
| 2731 | CAAGGGGGAGCATAT | A572729H | +C*+A*+A*G*G*G*G*G*A*G*C*A*T*A*T*+C | 74126 | 18 |
| | CCC | | *+C*+C | | |
| 2732 | TCAAGGGGGAGCATA | A572730H | +T*+C*+A*A*G*G*G*G*G*A*G*C*A*T*A*T*+ | 74126 | 19 |
| | TCCC | | C*+C*+C | | |
| 2733 | TCAAGGGGGAGCATA | A572731H | +T*+C*+A*A*G*G*G*G*G*A*G*C*A*T*A*+T* | 74127 | 18 |
| | TCC | | +C*+C | | |
| 2734 | CTCAAGGGGGAGCAT | A572732H | +C*+T*+C*A*A*G*G*G*G*G*A*G*C*A*T*A* | 74127 | 19 |
| | ATCC | | +T*+C*+C | | |
| 2735 | CTCAAGGGGGAGCAT | A572733H | +C*+T*+C*A*A*G*G*G*G*G*A*G*C*A*T*+A | 74128 | 18 |
| | ATC | | *+T*+C | | |
| 2736 | TCTCAAGGGGGAGCA | A572734H | +T*+C*+T*C*A*A*G*G*G*G*G*A*G*C*A*T*+ | 74128 | 19 |
| | TATC | | A*+T*+C | | |
| 2737 | TCTCAAGGGGGAGCA | A572735H | +T*+C*+T*C*A*A*G*G*G*G*G*A*G*C*A*+T* | 74129 | 18 |
| | TAT | | +A*+T | | |
| 2738 | GTCTCAAGGGGGAGC | A572736H | +G*+T*+C*T*C*A*A*G*G*G*G*G*A*G*C*A* | 74129 | 19 |
| | ATAT | | +T*+A*+T | | |
| 2739 | AGTCTCAAGGGGGAG | A572737H | +A*+G*+T*C*T*C*A*A*G*G*G*G*G*A*G*C* | 74130 | 19 |
| | CATA | | +A*+T*+A | | |
| 2740 | AGTCTCAAGGGGGAG | A572738H | +A*+G*+T*C*T*C*A*A*G*G*G*G*G*A*G*+C | 74131 | 18 |
| | CAT | | *+A*+T | | |
| 2741 | CAGTCTCAAGGGGGA | A572739H | +C*+A*+G*T*C*T*C*A*A*G*G*G*G*G*A*G* | 74131 | 19 |
| | GCAT | | +C*+A*+T | | |
| 2742 | CAGTCTCAAGGGGGA | A572740H | +C*+A*+G*T*C*T*C*A*A*G*G*G*G*G*A*+G | 74132 | 18 |
| | GCA | | *+C*+A | | |
| 2743 | TCAGTCTCAAGGGGG | A572741H | +T*+C*+A*G*T*C*T*C*A*A*G*G*G*G*G*A*+ | 74132 | 19 |
| | AGCA | | G*+C*+A | | |
| 2744 | TCAGTCTCAAGGGGG | A572742H | +T*+C*+A*G*T*C*T*C*A*A*G*G*G*G*G*+A* | 74133 | 18 |
| | AGC | | +G*+C | | |
| 2745 | TTCAGTCTCAAGGGG | A572743H | +T*+T*+C*A*G*T*C*T*C*A*A*G*G*G*G*G*+ | 74133 | 19 |
| | GAGC | | A*+G*+C | | |
| 2746 | CTTCAGTCTCAAGGG | A572744H | +C*+T*+T*C*A*G*T*C*T*C*A*A*G*G*G*G*+ | 74134 | 19 |
| | GGAG | | G*+A*+G | | |
| 2747 | CCTTCAGTCTCAAGG | A572745H | +C*+C*+T*T*C*A*G*T*C*T*C*A*A*G*G*G*+ | 74135 | 19 |
| | GGGA | | G*+G*+A | | |
| 2748 | TTCCTTCAGTCTCAA | A572746H | +T*+T*+C*C*T*T*C*A*G*T*C*T*C*A*A*G*+ | 74137 | 19 |
| | GGGG | | G*+G*+G | | |
| 2749 | GTTCCTTCAGTCTCA | A572747H | +G*+T*+T*C*C*T*T*C*A*G*T*C*T*C*A*A*+ | 74138 | 19 |
| | AGGG | | G*+G*+G | | |
| 2750 | AGTTCCTTCAGTCTC | A572748H | +A*+G*+T*T*C*C*T*T*C*A*G*T*C*T*C*A*+ | 74139 | 19 |
| | AAGG | | A*+G*+G | | |
| 2751 | CAGTTCCTTCAGTCT | A572749H | +C*+A*+G*T*T*C*C*T*T*C*A*G*T*C*T*C*+ | 74140 | 19 |
| | CAAG | | A*+A*+G | | |
| 2752 | TTACTAAGGTTTAAA | A572750H | +T*+T*+A*C*T*A*A*G*G*T*T*T*A*A*A*A*+ | 74160 | 19 |
| | ATCT | | T*+C*+T | | |
| 2753 | TCTGATAGGGATGAA | A572751H | +T*+C*+T*G*A*T*A*G*G*G*A*T*G*A*A*A*+ | 74184 | 19 |
| | ATGT | | T*+G*+T | | |
| 2754 | TCTCTGATAGGGATG | A572752H | +T*+C*+T*C*T*G*A*T*A*G*G*G*A*T*G*A*+ | 74186 | 19 |
| | AAAT | | A*+A*+T | | |
| 2755 | AGAAAGTTCTCTGAT | A572753H | +A*+G*+A*A*A*G*T*T*C*T*C*T*G*A*T*A*+ | 74193 | 19 |
| | AGGG | | G*+G*+G | | |
| 2756 | AGCCTCAAGAAAGTT | A572754H | +A*+G*+C*C*T*C*A*A*G*A*A*A*G*T*T*C*+ | 74200 | 19 |
| | CTCT | | T*+C*+T | | |
| 2757 | AAGCCTCAAGAAAGT | A572755H | +A*+A*+G*C*C*T*C*A*A*G*A*A*A*G*T*T*+ | 74201 | 19 |
| | TCTC | | C*+T*+C | | |
| 2758 | GACAAGCCTCAAGAA | A572756H | +G*+A*+C*A*A*G*C*C*T*C*A*A*G*A*A*A*+ | 74204 | 19 |
| | AGTT | | G*+T*+T | | |
| 2759 | GGACAAGCCTCAAGA | A572757H | +G*+G*+A*C*A*A*G*C*C*T*C*A*A*G*A*A*+ | 74205 | 19 |
| | AAGT | | A*+G*+T | | |
| 2760 | AGGACAAGCCTCAAG | A572758H | +A*+G*+G*A*C*A*A*G*C*C*T*C*A*A*G*A*+ | 74206 | 19 |
| | AAAG | | A*+A*+G | | |
| 2761 | CTTTAGGACAAGCCT | A572759H | +C*+T*+T*T*A*G*G*A*C*A*A*G*C*C*T*C*+ | 74210 | 19 |
| | CAAG | | A*+A*+G | | |
| 2762 | ACTTTAGGACAAGCC | A572760H | +A*+C*+T*T*T*A*G*G*A*C*A*A*G*C*C*T*+ | 74211 | 19 |
| | TCAA | | C*+A*+A | | |
| 2763 | ACTTTAGGACAAGCC | A572761H | +A*+C*+T*T*T*A*G*G*A*C*A*A*G*C*C*+T* | 74212 | 18 |
| | TCA | | +C*+A | | |
| 2764 | CACTTTAGGACAAGC | A572762H | +C*+A*+C*T*T*T*A*G*G*A*C*A*A*G*C*C*+ | 74212 | 19 |
| | CTCA | | T*+C*+A | | |
| 2765 | CCACTTTAGGACAAG | A572763H | +C*+C*+A*C*T*T*T*A*G*G*A*C*A*A*G*C*+ | 74213 | 19 |
| | CCTC | | C*+T*+C | | |
| 2766 | TCCACTTTAGGACAA | A572764H | +T*+C*+C*A*C*T*T*T*A*G*G*A*C*A*A*G*+ | 74214 | 19 |
| | GCCT | | C*+C*+T | | |
| 2767 | CTCCACTTTAGGACA | A572765H | +C*+T*+C*C*A*C*T*T*T*A*G*G*A*C*A*A*+ | 74215 | 19 |
| | AGCC | | G*+C*+C | | |
| 2768 | CTCTCCACTTTAGGA | A572766H | +C*+T*+C*T*C*C*A*C*T*T*T*A*G*G*A*C*+ | 74217 | 19 |
| | CAAG | | A*+A*+G | | |
| 2769 | CTCTCCACTTTAGGA | A572767H | +C*+T*+C*T*C*C*A*C*T*T*T*A*G*G*A*+C* | 74218 | 18 |
| | CAA | | +A*+A | | |
| 2770 | ACTCTCCACTTTAGG | A572768H | +A*+C*+T*C*T*C*C*A*C*T*T*T*A*G*G*A*+ | 74218 | 19 |
| | ACAA | | C*+A*+A | | |
| 2771 | AACTCTCCACTTTAG | A572769H | +A*+A*+C*T*C*T*C*C*A*C*T*T*T*A*G*G*+ | 74219 | 19 |
| | GACA | | A*+C*+A | | |
| 2772 | AAACTCTCCACTTTA | A572770H | +A*+A*+A*C*T*C*T*C*C*A*C*T*T*T*A*G*+ | 74220 | 19 |
| | GGAC | | G*+A*+C | | |
| 2773 | GAAACTCTCCACTTT | A572771H | +G*+A*+A*A*C*T*C*T*C*C*A*C*T*T*T*A*+ | 74221 | 19 |
| | AGGA | | G*+G*+A | | |
| 2774 | TGAAACTCTCCACTT | A572772H | +T*+G*+A*A*A*C*T*C*T*C*C*A*C*T*T*T*+ | 74222 | 19 |
| | TAGG | | A*+G*+G | | |
| 2775 | CTGAAACTCTCCACT | A572773H | +C*+T*+G*A*A*A*C*T*C*T*C*C*A*C*T*T*+ | 74223 | 19 |
| | TTAG | | T*+A*+G | | |
| 2776 | GCACTGAAACTCTCC | A572774H | +G*+C*+A*C*T*G*A*A*A*C*T*C*T*C*C*A*+ | 74226 | 19 |
| | ACTT | | C*+T*+T | | |
| 2777 | GGCACTGAAACTCTC | A572775H | +G*+G*+C*A*C*T*G*A*A*A*C*T*C*T*C*C*+ | 74227 | 19 |
| | CACT | | A*+C*+T | | |
| 2778 | CTGGCACTGAAACTC | A572776H | +C*+T*+G*G*C*A*C*T*G*A*A*A*C*T*C*T*+ | 74229 | 19 |
| | TCCA | | C*+C*+A | | |
| 2779 | TCTGGCACTGAAACT | A572777H | +T*+C*+T*G*G*C*A*C*T*G*A*A*A*C*T*C*+ | 74230 | 19 |
| | CTCC | | T*+C*+C | | |
| 2780 | CCTGGTAAGGAGGAG | A572778Hi | +C*+C*+T*G*G*T*A*A*G*G*A*G*G*A*G*+A | 74278 | 18 |
| | ACC | | *+C*+C | | |
| 2781 | GCCTGGTAAGGAGGA | A572779Hi | +G*+C*+C*T*G*G*T*A*A*G*G*A*G*G*A*G* | 74278 | 19 |
| | GACC | | +A*+C*+C | | |
| 2782 | ATCTTGTACAACTTA | A572780Hi | +A*+T*+C*T*T*G*T*A*C*A*A*C*T*T*A*G*+ | 74297 | 19 |
| | GAGG | | A*+G*+G | | |
| 2783 | TCTTGTACAACTTAG | A572781Hi | +T*+C*+T*T*G*T*A*C*A*A*C*T*T*A*G*+A* | 74297 | 18 |
| | AGG | | +G*+G | | |
| 2784 | CATCTTGTACAACTT | A572782Hi | +C*+A*+T*C*T*T*G*T*A*C*A*A*C*T*T*A*+ | 74298 | 19 |
| | AGAG | | G*+A*+G | | |
| 2785 | ACTCATCTTGTACAA | A572783Hi | +A*+C*+T*C*A*T*C*T*T*G*T*A*C*A*A*+C* | 74302 | 18 |
| | CTT | | +T*+T | | |
| 2786 | AGCATAATATTTAAA | A572784Hi | +A*+G*+C*A*T*A*A*T*A*T*T*T*A*A*A*C*+ | 74360 | 19 |
| | CTGA | | T*+G*+A | | |
| 2787 | CAGCATAATATTTAA | A572785Hi | +C*+A*+G*C*A*T*A*A*T*A*T*T*T*A*A*A*+ | 74361 | 19 |
| | ACTG | | C*+T*+G | | |
| 2788 | TCCAGCATAATATTT | A572786Hi | +T*+C*+C*A*G*C*A*T*A*A*T*A*T*T*T*A*+ | 74363 | 19 |
| | AAAC | | A*+A*+C | | |
| 2789 | ATGAGCCAATATTTG | A572787Hi | +A*+T*+G*A*G*C*C*A*A*T*A*T*T*T*G*+A* | 74753 | 18 |
| | ACA | | +C*+A | | |
| 2790 | GTTTGGCACAGTTGT | A572788Hi | +G*+T*+T*T*G*G*C*A*C*A*G*T*T*G*T*+T* | 74776 | 18 |
| | TCC | | +C*+C | | |
| 2791 | TAGTTTGGCACAGTT | A572789Hi | +T*+A*+G*T*T*T*G*G*C*A*C*A*G*T*T*G*+ | 74777 | 19 |
| | GTTC | | T*+T*+C | | |
| 2792 | ATTAGTTTGGCACAG | A572790Hi | +A*+T*+T*A*G*T*T*T*G*G*C*A*C*A*G*T*+ | 74779 | 19 |
| | TTGT | | T*+G*+T | | |
| 2793 | AATTTGCATTAGTTT | A572791Hi | +A*+A*+T*T*T*G*C*A*T*T*A*G*T*T*T*G*+ | 74786 | 19 |
| | GGCA | | G*+C*+A | | |
| 2794 | GCCAAGTAACTATCA | A572792Hi | +G*+C*+C*A*A*G*T*A*A*C*T*A*T*C*A*+T* | 74970 | 18 |
| | TTT | | +T*+T | | |
| 2795 | CAGTAGGCCAAGTAA | A572793Hi | +C*+A*+G*T*A*G*G*C*C*A*A*G*T*A*A*+C* | 74976 | 18 |
| | CTA | | +T*+A | | |
| 2796 | ACAGTAGGCCAAGTA | A572794Hi | +A*+C*+A*G*T*A*G*G*C*C*A*A*G*T*A*+A* | 74977 | 18 |
| | ACT | | +C*+T | | |
| 2797 | ACAGTAGGCCAAGTA | A572795Hi | +A*+C*+A*G*T*A*G*G*C*C*A*A*G*T*+A*+ | 74978 | 17 |
| | AC | | A*+C | | |
| 2798 | CACAGTAGGCCAAGT | A572796Hi | +C*+A*+C*A*G*T*A*G*G*C*C*A*A*G*T*+A* | 74978 | 18 |
| | AAC | | +A*+C | | |
| 2799 | TACACAGTAGGCCAA | A572797Hi | +T*+A*+C*A*C*A*G*T*A*G*G*C*C*A*A*G*+ | 74979 | 19 |
| | GTAA | | T*+A*+A | | |
| 2800 | ACACAGTAGGCCAAG | A572798Hi | +A*+C*+A*C*A*G*T*A*G*G*C*C*A*A*G*+T* | 74979 | 18 |
| | TAA | | +A*+A | | |
| 2801 | GCTGAAGAAACTTCA | A572799Hi | +G*+C*+T*G*A*A*G*A*A*A*C*T*T*C*A*G*+ | 75088 | 19 |
| | GTAT | | T*+A*+T | | |
| 2802 | AATGTCTTGCAAAGC | A572800Hi | +A*+A*+T*G*T*C*T*T*G*C*A*A*A*G*C*A*+ | 75212 | 19 |
| | ACAA | | C*+A*+A | | |
| 2803 | AACATAATTTCCTCTA | A572801Hi | +A*+A*+C*A*T*A*A*T*T*T*C*C*T*C*T*A*+ | 75304 | 19 |
| | TTA | | T*+T*+A | | |
| 2804 | TAGGATACAAATGAA | A572802Hi | +T*+A*+G*G*A*T*A*C*A*A*A*T*G*A*A*C*+ | 75317 | 19 |
| | CATA | | A*+T*+A | | |
| 2805 | TTAGGATACAAATGA | A572803Hi | +T*+T*+A*G*G*A*T*A*C*A*A*A*T*G*A*A*+ | 75318 | 19 |
| | ACAT | | C*+A*+T | | |
| 2806 | TCTCTATGAAGGAAG | A572804Hi | +T*+C*+T*C*T*A*T*G*A*A*G*G*A*A*G*A*+ | 75400 | 19 |
| | ATAA | | T*+A*+A | | |
| 2807 | TTCTCTATGAAGGAA | A572805Hi | +T*+T*+C*T*C*T*A*T*G*A*A*G*G*A*A*G*+ | 75401 | 19 |
| | GATA | | A*+T*+A | | |
| 2808 | ATTCTCTATGAAGGA | A572806Hi | +A*+T*+T*C*T*C*T*A*T*G*A*A*G*G*A*A*+ | 75402 | 19 |
| | AGAT | | G*+A*+T | | |
| 2809 | GAGTATTCTCTATGA | A572807Hi | +G*+A*+G*T*A*T*T*C*T*C*T*A*T*G*A*+A* | 75407 | 18 |
| | AGG | | +G*+G | | |
| 2810 | TACTGAGTCAGGTAT | A572808Hi | +T*+A*+C*T*G*A*G*T*C*A*G*G*T*A*T*A*+ | 75448 | 19 |
| | AGAA | | G*+A*+A | | |
| 2811 | TGCTATTAAATAGAA | A572809Hi | +T*+G*+C*T*A*T*T*A*A*A*T*A*G*A*A*T*+ | 75476 | 19 |
| | TTAA | | T*+A*+A | | |
| 2812 | CTGCTATTAAATAGA | A572810Hi | +C*+T*+G*C*T*A*T*T*A*A*A*T*A*G*A*A*+ | 75477 | 19 |
| | ATTA | | T*+T*+A | | |
| 2813 | TTTGATCATTATTAGT | A572811Hi | +T*+T*+T*G*A*T*C*A*T*T*A*T*T*A*G*T*+ | 75546 | 19 |
| | TTC | | T*+T*+C | | |
| 2814 | CTTTGATCATTATTAG | A572812Hi | +C*+T*+T*T*G*A*T*C*A*T*T*A*T*T*A*+G* | 75548 | 18 |
| | TT | | +T*+T | | |
| 2815 | GCTTTGATCATTATTA | A572813Hi | +G*+C*+T*T*T*G*A*T*C*A*T*T*A*T*T*+A* | 75549 | 18 |
| | GT | | +G*+T | | |
| 2816 | GATGGCTTTGATCAT | A572814Hi | +G*+A*+T*G*G*C*T*T*T*G*A*T*C*A*T*+T* | 75553 | 18 |
| | TAT | | +A*+T | | |
| 2817 | AAATTAAGATGGCTT | A572815Hi | +A*+A*+A*T*T*A*A*G*A*T*G*G*C*T*T*T*+ | 75559 | 19 |
| | TGAT | | G*+A*+T | | |
| 2818 | CAAATTAAGATGGCT | A572816Hi | +C*+A*+A*A*T*T*A*A*G*A*T*G*G*C*T*T*+ | 75560 | 19 |
| | TTGA | | T*+G*+A | | |
| 2819 | AGTTTGCTTTGCTTA | A572817Hi | +A*+G*+T*T*T*G*C*T*T*T*G*C*T*T*A*C*+ | 75688 | 19 |
| | CTAG | | T*+A*+G | | |
| 2820 | GAACTTAATCACAAG | A572818Hi | +G*+A*+A*C*T*T*A*A*T*C*A*C*A*A*G*T*+ | 75723 | 19 |
| | TTCC | | T*+C*+C | | |
| 2821 | TGAACTTAATCACAA | A572819Hi | +T*+G*+A*A*C*T*T*A*A*T*C*A*C*A*A*G*+ | 75724 | 19 |
| | GTTC | | T*+T*+C | | |
| 2822 | TGAACTTAATCACAA | A572820Hi | +T*+G*+A*A*C*T*T*A*A*T*C*A*C*A*A*+G* | 75725 | 18 |
| | GTT | | +T*+T | | |
| 2823 | TCTGAACTTAATCAC | A572821Hi | +T*+C*+T*G*A*A*C*T*T*A*A*T*C*A*C*A*+ | 75726 | 19 |
| | AAGT | | A*+G*+T | | |
| 2824 | TTACTTAATGTTTGTC | A572822Hi | +T*+T*+A*C*T*T*A*A*T*G*T*T*T*G*T*C*+ | 75747 | 19 |
| | TGT | | T*+G*+T | | |
| 2825 | CATCATTACTTAATGT | A572823Hi | +C*+A*+T*C*A*T*T*A*C*T*T*A*A*T*G*T*+ | 75752 | 19 |
| | TTG | | T*+T*+G | | |
| 2826 | GCCATCATTACTTAA | A572824Hi | +G*+C*+C*A*T*C*A*T*T*A*C*T*T*A*A*T*+ | 75754 | 19 |
| | TGTT | | G*+T*+T | | |
| 2827 | TACCACAGAGCATCC | A572825Hi | +T*+A*+C*C*A*C*A*G*A*G*C*A*T*C*C*T*+ | 75818 | 19 |
| | TGAA | | G*+A*+A | | |
| 2828 | CTGAGGTACCACAGA | A572826Hi | +C*+T*+G*A*G*G*T*A*C*C*A*C*A*G*A*G*+ | 75824 | 19 |
| | GCAT | | C*+A*+T | | |
| 2829 | ACTCCACAGTCTGAA | A572827Hi | +A*+C*+T*C*C*A*C*A*G*T*C*T*G*A*A*T*+ | 75849 | 19 |
| | TCAG | | C*+A*+G | | |
| 2830 | TGACCATACACAAGC | A572828Hi | +T*+G*+A*C*C*A*T*A*C*A*C*A*A*G*C*T*+ | 75935 | 19 |
| | TTCA | | T*+C*+A | | |
| 2831 | CACTGACCATACACA | A572829Hi | +C*+A*+C*T*G*A*C*C*A*T*A*C*A*C*A*A*+ | 75938 | 19 |
| | AGCT | | G*+C*+T | | |
| 2832 | ACACTGACCATACAC | A572830Hi | +A*+C*+A*C*T*G*A*C*C*A*T*A*C*A*C*A*+ | 75939 | 19 |
| | AAGC | | A*+G*+C | | |
| 2833 | ACACACTGACCATAC | A572831Hi | +A*+C*+A*C*A*C*T*G*A*C*C*A*T*A*C*A*+ | 75941 | 19 |
| | ACAA | | C*+A*+A | | |
| 2834 | GACACACTGACCATA | A572832Hi | +G*+A*+C*A*C*A*C*T*G*A*C*C*A*T*A*C*+ | 75942 | 19 |
| | CACA | | A*+C*+A | | |
| 2835 | AGATGACACACTGAC | A572833Hi | +A*+G*+A*T*G*A*C*A*C*A*C*T*G*A*C*C*+ | 75946 | 19 |
| | CATA | | A*+T*+A | | |
| 2836 | GTGAGATTCAGCTGA | A572834Hi | +G*+T*+G*A*G*A*T*T*C*A*G*C*T*G*A*G*+ | 75960 | 19 |
| | GATG | | A*+T*+G | | |
| 2837 | GGATCATTGTGAGAT | A572835Hi | +G*+G*+A*T*C*A*T*T*G*T*G*A*G*A*T*T*+ | 75968 | 19 |
| | TCAG | | C*+A*+G | | |
| 2838 | CATTAGGATCATTGT | A572836Hi | +C*+A*+T*T*A*G*G*A*T*C*A*T*T*G*T*G*+ | 75973 | 19 |
| | GAGA | | A*+G*+A | | |
| 2839 | CCATTAGGATCATTG | A572837Hi | +C*+C*+A*T*T*A*G*G*A*T*C*A*T*T*G*T*+ | 75974 | 19 |
| | TGAG | | G*+A*+G | | |
| 2840 | CCATTAGGATCATTG | A572838Hi | +C*+C*+A*T*T*A*G*G*A*T*C*A*T*T*G*+T* | 75975 | 18 |
| | TGA | | +G*+A | | |
| 2841 | ACCATTAGGATCATT | A572839Hi | +A*+C*+C*A*T*T*A*G*G*A*T*C*A*T*T*G*+ | 75975 | 19 |
| | GTGA | | T*+G*+A | | |
| 2842 | GTCATACCTGTCCTA | A572840Hi | +G*+T*+C*A*T*A*C*C*T*G*T*C*C*T*+A*+G | 75998 | 17 |
| | GG | | *+G | | |
| 2843 | GAGGTCATACCTGTC | A572841Hi | +G*+A*+G*G*T*C*A*T*A*C*C*T*G*T*C*C*+ | 75999 | 19 |
| | CTAG | | T*+A*+G | | |
| 2844 | GTAGTGCAAAGCAAA | A572842Hi | +G*+T*+A*G*T*G*C*A*A*A*G*C*A*A*A*C*+ | 76123 | 19 |
| | CAGA | | A*+G*+A | | |
| 2845 | TGTAGTGCAAAGCAA | A572843Hi | +T*+G*+T*A*G*T*G*C*A*A*A*G*C*A*A*A*+ | 76124 | 19 |
| | ACAG | | C*+A*+G | | |
| 2846 | TTGTGTAGTGCAAAG | A572844Hi | +T*+T*+G*T*G*T*A*G*T*G*C*A*A*A*G*C*+ | 76127 | 19 |
| | CAAA | | A*+A*+A | | |
| 2847 | GCATCTTCTCAAATG | A572845Hi | +G*+C*+A*T*C*T*T*C*T*C*A*A*A*T*G*T*+ | 76168 | 19 |
| | TAAG | | A*+A*+G | | |
| 2848 | GGCATCTTCTCAAAT | A572846Hi | +G*+G*+C*A*T*C*T*T*C*T*C*A*A*A*T*G*+ | 76169 | 19 |
| | GTAA | | T*+A*+A | | |
| 2849 | GAGTCTTGAGTTTCA | A572847Hi | +G*+A*+G*T*C*T*T*G*A*G*T*T*T*C*A*T*+ | 76505 | 19 |
| | TATG | | A*+T*+G | | |
| 2850 | TTCAGAGTCTTGAGT | A572848Hi | +T*+T*+C*A*G*A*G*T*C*T*T*G*A*G*T*T*+ | 76509 | 19 |
| | TTCA | | T*+C*+A | | |
| 2851 | CTTCAGAGTCTTGAG | A572849Hi | +C*+T*+T*C*A*G*A*G*T*C*T*T*G*A*G*T*+ | 76510 | 19 |
| | TTTC | | T*+T*+C | | |
| 2852 | ACTTCAGAGTCTTGA | A572850Hi | +A*+C*+T*T*C*A*G*A*G*T*C*T*T*G*A*G*+ | 76511 | 19 |
| | GTTT | | T*+T*+T | | |
| 2853 | AAGACTTCAGAGTCT | A572851Hi | +A*+A*+G*A*C*T*T*C*A*G*A*G*T*C*T*T*+ | 76514 | 19 |
| | TGAG | | G*+A*+G | | |
| 2854 | TAAGACTTCAGAGTC | A572852Hi | +T*+A*+A*G*A*C*T*T*C*A*G*A*G*T*C*T*+ | 76515 | 19 |
| | TTGA | | T*+G*+A | | |
| 2855 | TAAGACTTCAGAGTC | A572853Hi | +T*+A*+A*G*A*C*T*T*C*A*G*A*G*T*C*+T* | 76516 | 18 |
| | TTG | | +T*+G | | |
| 2856 | ACTTAAGACTTCAGA | A572854Hi | +A*+C*+T*T*A*A*G*A*C*T*T*C*A*G*A*G*+ | 76518 | 19 |
| | GTCT | | T*+C*+T | | |
| 2857 | ATTCTCCACTTAAGA | A572855Hi | +A*+T*+T*C*T*C*C*A*C*T*T*A*A*G*A*C*+ | 76525 | 19 |
| | CTTC | | T*+T*+C | | |
| 2858 | TGATTCTCCACTTAA | A572856Hi | +T*+G*+A*T*T*C*T*C*C*A*C*T*T*A*A*G*+ | 76527 | 19 |
| | GACT | | A*+C*+T | | |
| 2859 | ATGATTCTCCACTTA | A572857Hi | +A*+T*+G*A*T*T*C*T*C*C*A*C*T*T*A*A*+ | 76528 | 19 |
| | AGAC | | G*+A*+C | | |
| 2860 | AATGATTCTCCACTT | A572858Hi | +A*+A*+T*G*A*T*T*C*T*C*C*A*C*T*T*A*+ | 76529 | 19 |
| | AAGA | | A*+G*+A | | |
| 2861 | AGCTGTCTTCCTGAG | A572859Hi | +A*+G*+C*T*G*T*C*T*T*C*C*T*G*A*G*A*+ | 76596 | 19 |
| | AAGC | | A*+G*+C | | |
| 2862 | AGCTGTCTTCCTGAG | A572860Hi | +A*+G*+C*T*G*T*C*T*T*C*C*T*G*A*G*+A* | 76597 | 18 |
| | AAG | | +A*+G | | |
| 2863 | AAGCTGTCTTCCTGA | A572861Hi | +A*+A*+G*C*T*G*T*C*T*T*C*C*T*G*A*G*+ | 76597 | 19 |
| | GAAG | | A*+A*+G | | |
| 2864 | GAAGCTGTCTTCCTG | A572862Hi | +G*+A*+A*G*C*T*G*T*C*T*T*C*C*T*G*A*+ | 76598 | 19 |
| | AGAA | | G*+A*+A | | |
| 2865 | AGAAGCTGTCTTCCT | A572863Hi | +A*+G*+A*A*G*C*T*G*T*C*T*T*C*C*T*G*+ | 76599 | 19 |
| | GAGA | | A*+G*+A | | |
| 2866 | ATCTAGACAATCTTC | A572864Hi | +A*+T*+C*T*A*G*A*C*A*A*T*C*T*T*C*A*+ | 76642 | 19 |
| | AAGG | | A*+G*+G | | |
| 2867 | TTATCTAGACAATCTT | A572865Hi | +T*+T*+A*T*C*T*A*G*A*C*A*A*T*C*T*T*+ | 76644 | 19 |
| | CAA | | C*+A*+A | | |
| 2868 | TTTATCTAGACAATCT | A572866Hi | +T*+T*+T*A*T*C*T*A*G*A*C*A*A*T*C*+T* | 76646 | 18 |
| | TC | | +T*+C | | |
| 2869 | GTATGAGGTACCTCC | A572867Hi | +G*+T*+A*T*G*A*G*G*T*A*C*C*T*C*C*+A* | 76853 | 18 |
| | AAG | | +A*+G | | |
| 2870 | AGTATGAGGTACCTC | A572868Hi | +A*+G*+T*A*T*G*A*G*G*T*A*C*C*T*C*C*+ | 76853 | 19 |
| | CAAG | | A*+A*+G | | |
| 2871 | AGTATGAGGTACCTC | A572869Hi | +A*+G*+T*A*T*G*A*G*G*T*A*C*C*T*C*+C* | 76854 | 18 |
| | CAA | | +A*+A | | |
| 2872 | GGAAATGCAGTATGA | A572870Hi | +G*+G*+A*A*A*T*G*C*A*G*T*A*T*G*A*G*+ | 76861 | 19 |
| | GGTA | | G*+T*+A | | |
| 2873 | TTAAGAAGGAAATGC | A572871Hi | +T*+T*+A*A*G*A*A*G*G*A*A*A*T*G*C*A*+ | 76868 | 19 |
| | AGTA | | G*+T*+A | | |
| 2874 | GTTCTATGGAATAAT | A572872Hi | +G*+T*+T*C*T*A*T*G*G*A*A*T*A*A*T*A*+ | 77334 | 19 |
| | AATG | | A*+T*+G | | |
| 2875 | ATAAAGAGTGTTCTA | A572873Hi | +A*+T*+A*A*A*G*A*G*T*G*T*T*C*T*A*T*+ | 77343 | 19 |
| | TGGA | | G*+G*+A | | |
| 2876 | CATAAAGAGTGTTCT | A572874Hi | +C*+A*+T*A*A*A*G*A*G*T*G*T*T*C*T*A*+ | 77344 | 19 |
| | ATGG | | T*+G*+G | | |
| 2877 | TCATAAAGAGTGTTC | A572875Hi | +T*+C*+A*T*A*A*A*G*A*G*T*G*T*T*C*T*+ | 77345 | 19 |
| | TATG | | A*+T*+G | | |
| 2878 | TCATAAAGAGTGTTC | A572876Hi | +T*+C*+A*T*A*A*A*G*A*G*T*G*T*T*C*+T* | 77346 | 18 |
| | TAT | | +A*+T | | |
| 2879 | CTCATAAAGAGTGTT | A572877Hi | +C*+T*+C*A*T*A*A*A*G*A*G*T*G*T*T*C*+ | 77346 | 19 |
| | CTAT | | T*+A*+T | | |
| 2880 | ACTAGCTCATAAAGA | A572878Hi | +A*+C*+T*A*G*C*T*C*A*T*A*A*A*G*A*G*+ | 77351 | 19 |
| | GTGT | | T*+G*+T | | |
| 2881 | CAAATTATGTGTGAT | A572879Hi | +C*+A*+A*A*T*T*A*T*G*T*G*T*G*A*T*G*+ | 77479 | 19 |
| | GTTA | | T*+T*+A | | |
| 2882 | CCAACATTTGTACAA | A572880Hi | +C*+C*+A*A*C*A*T*T*T*G*T*A*C*A*A*A*+ | 77520 | 19 |
| | AGTA | | G*+T*+A | | |
| 2883 | GAAGCTAAACAGCTA | A572881Hi | +G*+A*+A*G*C*T*A*A*A*C*A*G*C*T*A*T*+ | 77556 | 19 |
| | TTTA | | T*+T*+A | | |
| 2884 | ACCTGGAAGCTAAAC | A572882Hi | +A*+C*+C*T*G*G*A*A*G*C*T*A*A*A*C*A*+ | 77561 | 19 |
| | AGCT | | G*+C*+T | | |
| 2885 | AATACTTCCACTAAA | A572883Hi | +A*+A*+T*A*C*T*T*C*C*A*C*T*A*A*A*C*+ | 77613 | 19 |
| | CAAT | | A*+A*+T | | |
| 2886 | CCATTCTCACACTGA | A572884Hi | +C*+C*+A*T*T*C*T*C*A*C*A*C*T*G*A*C*+ | 77670 | 19 |
| | CAAT | | A*+A*+T | | |
| 2887 | GCAAGAAAGGCATAG | A572885H | +G*+C*+A*A*G*A*A*A*G*G*C*A*T*A*G*+C | 77793 | 18 |
| | CAG | | *+A*+G | | |
| 2888 | GGCAAGAAAGGCATA | A572886H | +G*+G*+C*A*A*G*A*A*A*G*G*C*A*T*A*G* | 77793 | 19 |
| | GCAG | | +C*+A*+G | | |
| 2889 | GAGGCAAGAAAGGCA | A572887H | +G*+A*+G*G*C*A*A*G*A*A*A*G*G*C*A*T* | 77795 | 19 |
| | TAGC | | +A*+G*+C | | |
| 2890 | AAAATTGCTTCTGGA | A572888H | +A*+A*+A*A*T*T*G*C*T*T*C*T*G*G*A*A*+ | 77856 | 19 |
| | ATAC | | T*+A*+C | | |
| 2891 | TCTAAAGGTTCTGTT | A572889Hi | +T*+C*+T*A*A*A*G*G*T*T*C*T*G*T*T*C*+ | 77925 | 19 |
| | CCAG | | C*+A*+G | | |
| 2892 | AATCATGTTTCTAAA | A572890Hi | +A*+A*+T*C*A*T*G*T*T*T*C*T*A*A*A*G*+ | 77934 | 19 |
| | GGTT | | G*+T*+T | | |
| 2893 | ACACAATGATCCACT | A572891Hi | +A*+C*+A*C*A*A*T*G*A*T*C*C*A*C*T*+A* | 78078 | 18 |
| | AAA | | +A*+A | | |
| 2894 | TACACAATGATCCAC | A572892Hi | +T*+A*+C*A*C*A*A*T*G*A*T*C*C*A*C*T*+ | 78078 | 19 |
| | TAAA | | A*+A*+A | | |
| 2895 | ATAGCCAGTATAATC | A572893Hi | +A*+T*+A*G*C*C*A*G*T*A*T*A*A*T*C*T*+ | 78144 | 19 |
| | TCAT | | C*+A*+T | | |
| 2896 | GATAGCCAGTATAAT | A572894Hi | +G*+A*+T*A*G*C*C*A*G*T*A*T*A*A*T*C*+ | 78145 | 19 |
| | CTCA | | T*+C*+A | | |
| 2897 | GATAGCCAGTATAAT | A572895Hi | +G*+A*+T*A*G*C*C*A*G*T*A*T*A*A*T*+C* | 78146 | 18 |
| | CTC | | +T*+C | | |
| 2898 | GTGATAGCCAGTATA | A572896Hi | +G*+T*+G*A*T*A*G*C*C*A*G*T*A*T*A*+A* | 78148 | 18 |
| | ATC | | +T*+C | | |
| 2899 | AGTGATAGCCAGTAT | A572897Hi | +A*+G*+T*G*A*T*A*G*C*C*A*G*T*A*T*A*+ | 78148 | 19 |
| | AATC | | A*+T*+C | | |
| 2900 | TTAAAGTGATAGCCA | A572898Hi | +T*+T*+A*A*A*G*T*G*A*T*A*G*C*C*A*+G* | 78153 | 18 |
| | GTA | | +T*+A | | |
| 2901 | GTTAAAGTGATAGCC | A572899Hi | +G*+T*+T*A*A*A*G*T*G*A*T*A*G*C*C*A*+ | 78153 | 19 |
| | AGTA | | G*+T*+A | | |
| 2902 | ACTACATAAACTTGC | A572900Hi | +A*+C*+T*A*C*A*T*A*A*A*C*T*T*G*C*C*+ | 78273 | 19 |
| | CTAA | | T*+A*+A | | |
| 2903 | GAACTACATAAACTT | A572901Hi | +G*+A*+A*C*T*A*C*A*T*A*A*A*C*T*T*G*+ | 78275 | 19 |
| | GCCT | | C*+C*+T | | |
| 2904 | ACCTGGAACTACATA | A572902Hi | +A*+C*+C*T*G*G*A*A*C*T*A*C*A*T*A*+A* | 78281 | 18 |
| | AAC | | +A*+C | | |
| 2905 | TTTGGTTTATCATGTA | A572903Hi | +T*+T*+T*G*G*T*T*T*A*T*C*A*T*G*T*A*+ | 78410 | 19 |
| | TGA | | T*+G*+A | | |
| 2906 | TATGAATTTGGTTTAT | A572904Hi | +T*+A*+T*G*A*A*T*T*T*G*G*T*T*T*A*T*+ | 78416 | 19 |
| | CAT | | C*+A*+T | | |
| 2907 | GAATAGCTGACAGCA | A572905Hi | +G*+A*+A*T*A*G*C*T*G*A*C*A*G*C*A*A*+ | 78469 | 19 |
| | AGAA | | G*+A*+A | | |
| 2908 | GCATCATGCAAGCAG | A572906Hi | +G*+C*+A*T*C*A*T*G*C*A*A*G*C*A*G*C*+ | 78523 | 19 |
| | CATT | | A*+T*+T | | |
| 2909 | AGCATCATGCAAGCA | A572907Hi | +A*+G*+C*A*T*C*A*T*G*C*A*A*G*C*A*G*+ | 78524 | 19 |
| | GCAT | | C*+A*+T | | |
| 2910 | AGCTCATTGCAGCAT | A572908Hi | +A*+G*+C*T*C*A*T*T*G*C*A*G*C*A*T*C*+ | 78534 | 19 |
| | CATG | | A*+T*+G | | |
| 2911 | GAAGAGCTCATTGCA | A572909Hi | +G*+A*+A*G*A*G*C*T*C*A*T*T*G*C*A*G*+ | 78538 | 19 |
| | GCAT | | C*+A*+T | | |
| 2912 | CACAGAAGAGCTCAT | A572910Hi | +C*+A*+C*A*G*A*A*G*A*G*C*T*C*A*T*T*+ | 78542 | 19 |
| | TGCA | | G*+C*+A | | |
| 2913 | TCACAGAAGAGCTCA | A572911Hi | +T*+C*+A*C*A*G*A*A*G*A*G*C*T*C*A*T*+ | 78543 | 19 |
| | TTGC | | T*+G*+C | | |
| 2914 | ACACTCTCTATGGCT | A572912Hi | +A*+C*+A*C*T*C*T*C*T*A*T*G*G*C*T*+A* | 78591 | 18 |
| | AAA | | +A*+A | | |
| 2915 | TAACACTCTCTATGG | A572913Hi | +T*+A*+A*C*A*C*T*C*T*C*T*A*T*G*G*+C* | 78593 | 18 |
| | CTA | | +T*+A | | |
| 2916 | GTAACACTCTCTATG | A572914Hi | +G*+T*+A*A*C*A*C*T*C*T*C*T*A*T*G*+G* | 78594 | 18 |
| | GCT | | +C*+T | | |
| 2917 | TGTAACACTCTCTAT | A572915Hi | +T*+G*+T*A*A*C*A*C*T*C*T*C*T*A*T*+G* | 78595 | 18 |
| | GGC | | +G*+C | | |
| 2918 | GTTTGTGTAACACTC | A572916Hi | +G*+T*+T*T*G*T*G*T*A*A*C*A*C*T*C*T*+ | 78599 | 19 |
| | TCTA | | C*+T*+A | | |
| 2919 | TTATAGGTTTGTGTA | A572917Hi | +T*+T*+A*T*A*G*G*T*T*T*G*T*G*T*A*A*+ | 78605 | 19 |
| | ACAC | | C*+A*+C | | |
| 2920 | TTGAGAGCCTGAAGT | A572918Hi | +T*+T*+G*A*G*A*G*C*C*T*G*A*A*G*T*T*+ | 78792 | 19 |
| | TGAA | | G*+A*+A | | |
| 2921 | ATTTGAGAGCCTGAA | A572919Hi | +A*+T*+T*T*G*A*G*A*G*C*C*T*G*A*A*G*+ | 78794 | 19 |
| | GTTG | | T*+T*+G | | |
| 2922 | GATTTGAGAGCCTGA | A572920Hi | +G*+A*+T*T*T*G*A*G*A*G*C*C*T*G*A*A*+ | 78795 | 19 |
| | AGTT | | G*+T*+T | | |
| 2923 | ATGAGTATGCATGTG | A572921Hi | +A*+T*+G*A*G*T*A*T*G*C*A*T*G*T*G*A*+ | 78809 | 19 |
| | ATTT | | T*+T*+T | | |
| 2924 | GCATTATTGACATAT | A572922Hi | +G*+C*+A*T*T*A*T*T*G*A*C*A*T*A*T*+G* | 78823 | 18 |
| | GAG | | +A*+G | | |
| 2925 | AGCATTATTGACATA | A572923Hi | +A*+G*+C*A*T*T*A*T*T*G*A*C*A*T*A*T*+ | 78823 | 19 |
| | TGAG | | G*+A*+G | | |
| 2926 | CTAGCATTATTGACA | A572924Hi | +C*+T*+A*G*C*A*T*T*A*T*T*G*A*C*A*+T* | 78826 | 18 |
| | TAT | | +A*+T | | |
| 2927 | ATATCTAAAGACCGC | A572925H | +A*+T*+A*T*C*T*A*A*A*G*A*C*C*G*C*+A* | 78952 | 18 |
| | AAG | | +A*+G | | |
| 2928 | CATATCTAAAGACCG | A572926H | +C*+A*+T*A*T*C*T*A*A*A*G*A*C*C*G*C*+ | 78952 | 19 |
| | CAAG | | A*+A*+G | | |
| 2929 | CATATCTAAAGACCG | A572927H | +C*+A*+T*A*T*C*T*A*A*A*G*A*C*C*G*+C* | 78953 | 18 |
| | CAA | | +A*+A | | |
| 2930 | TCATATCTAAAGACC | A572928H | +T*+C*+A*T*A*T*C*T*A*A*A*G*A*C*C*G*+ | 78953 | 19 |
| | GCAA | | C*+A*+A | | |
| 2931 | GCTCATATCTAAAGA | A572929H | +G*+C*+T*C*A*T*A*T*C*T*A*A*A*G*A*C*+ | 78955 | 19 |
| | CCGC | | C*+G*+C | | |
| 2932 | GCTCATATCTAAAGA | A572930H | +G*+C*+T*C*A*T*A*T*C*T*A*A*A*G*A*+C* | 78956 | 18 |
| | CCG | | +C*+G | | |
| 2933 | TGCTCATATCTAAAG | A572931H | +T*+G*+C*T*C*A*T*A*T*C*T*A*A*A*G*A*+ | 78956 | 19 |
| | ACCG | | C*+C*+G | | |
| 2934 | CTGCTCATATCTAAA | A572932H | +C*+T*+G*C*T*C*A*T*A*T*C*T*A*A*A*G*+ | 78957 | 19 |
| | GACC | | A*+C*+C | | |
| 2935 | CTGCTCATATCTAAA | A572933H | +C*+T*+G*C*T*C*A*T*A*T*C*T*A*A*A*+G* | 78958 | 18 |
| | GAC | | +A*+C | | |
| 2936 | GCTGCTCATATCTAA | A572934H | +G*+C*+T*G*C*T*C*A*T*A*T*C*T*A*A*A*+ | 78958 | 19 |
| | AGAC | | G*+A*+C | | |
| 2937 | TGCTGCTCATATCTA | A572935H | +T*+G*+C*T*G*C*T*C*A*T*A*T*C*T*A*A*+ | 78959 | 19 |
| | AAGA | | A*+G*+A | | |
| 2938 | TTGCTGCTCATATCT | A572936H | +T*+T*+G*C*T*G*C*T*C*A*T*A*T*C*T*A*+ | 78960 | 19 |
| | AAAG | | A*+A*+G | | |
| 2939 | CATTGCTGCTCATAT | A572937H | +C*+A*+T*T*G*C*T*G*C*T*C*A*T*A*T*C*+ | 78962 | 19 |
| | CTAA | | T*+A*+A | | |
| 2940 | ATCATTGCTGCTCAT | A572938H | +A*+T*+C*A*T*T*G*C*T*G*C*T*C*A*T*A*+ | 78964 | 19 |
| | ATCT | | T*+C*+T | | |
| 2941 | TTCAAAGATTTCCAG | A572939H | +T*+T*+C*A*A*A*G*A*T*T*T*C*C*A*G*T*+ | 79005 | 19 |
| | TGTG | | G*+T*+G | | |
| 2942 | GTTCAAAGATTTCCA | A572940H | +G*+T*+T*C*A*A*A*G*A*T*T*T*C*C*A*G*+ | 79006 | 19 |
| | GTGT | | T*+G*+T | | |
| 2943 | TAAGTTCAAAGATTT | A572941H | +T*+A*+A*G*T*T*C*A*A*A*G*A*T*T*T*C*+ | 79009 | 19 |
| | CCAG | | C*+A*+G | | |
| 2944 | TTAAGTTCAAAGATT | A572942H | +T*+T*+A*A*G*T*T*C*A*A*A*G*A*T*T*T*+ | 79010 | 19 |
| | TCCA | | C*+C*+A | | |
| 2945 | CTTAAGTTCAAAGAT | A572943H | +C*+T*+T*A*A*G*T*T*C*A*A*A*G*A*T*T*+ | 79011 | 19 |
| | TTCC | | T*+C*+C | | |
| 2946 | CCTTAAGTTCAAAGA | A572944H | +C*+C*+T*T*A*A*G*T*T*C*A*A*A*G*A*T*+ | 79012 | 19 |
| | TTTC | | T*+T*+C | | |
| 2947 | CCCTTAAGTTCAAAG | A572945H | +C*+C*+C*T*T*A*A*G*T*T*C*A*A*A*G*A*+ | 79013 | 19 |
| | ATTT | | T*+T*+T | | |
| 2948 | TTCCCTTAAGTTCAA | A572946H | +T*+T*+C*C*C*T*T*A*A*G*T*T*C*A*A*A*+ | 79015 | 19 |
| | AGAT | | G*+A*+T | | |
| 2949 | AGTTCCCTTAAGTTC | A572947H | +A*+G*+T*T*C*C*C*T*T*A*A*G*T*T*C*A*+ | 79017 | 19 |
| | AAAG | | A*+A*+G | | |
| 2950 | GAGTTCCCTTAAGTT | A572948H | +G*+A*+G*T*T*C*C*C*T*T*A*A*G*T*T*C*+ | 79018 | 19 |
| | CAAA | | A*+A*+A | | |
| 2951 | AGAGTTCCCTTAAGT | A572949H | +A*+G*+A*G*T*T*C*C*C*T*T*A*A*G*T*T*+ | 79019 | 19 |
| | TCAA | | C*+A*+A | | |
| 2952 | AGAGTTCCCTTAAGT | A572950H | +A*+G*+A*G*T*T*C*C*C*T*T*A*A*G*T*+T* | 79020 | 18 |
| | TCA | | +C*+A | | |
| 2953 | AAGAGTTCCCTTAAG | A572951H | +A*+A*+G*A*G*T*T*C*C*C*T*T*A*A*G*T*+ | 79020 | 19 |
| | TTCA | | T*+C*+A | | |
| 2954 | TAAGAGTTCCCTTAA | A572952H | +T*+A*+A*G*A*G*T*T*C*C*C*T*T*A*A*G*+ | 79021 | 19 |
| | GTTC | | T*+T*+C | | |
| 2955 | CTAAATAAGAGTTCC | A572953H | +C*+T*+A*A*A*T*A*A*G*A*G*T*T*C*C*C*+ | 79026 | 19 |
| | CTTA | | T*+T*+A | | |
| 2956 | TGATTATGGCTAAAT | A572954H | +T*+G*+A*T*T*A*T*G*G*C*T*A*A*A*T*A*+ | 79035 | 19 |
| | AAGA | | A*+G*+A | | |
| 2957 | CTGATTATGGCTAAA | A572955H | +C*+T*+G*A*T*T*A*T*G*G*C*T*A*A*A*T*+ | 79036 | 19 |
| | TAAG | | A*+A*+G | | |
| 2958 | TCTGATTATGGCTAA | A572956H | +T*+C*+T*G*A*T*T*A*T*G*G*C*T*A*A*A*+ | 79037 | 19 |
| | ATAA | | T*+A*+A | | |
| 2959 | TCTGATTATGGCTAA | A572957H | +T*+C*+T*G*A*T*T*A*T*G*G*C*T*A*A*+A* | 79038 | 18 |
| | ATA | | +T*+A | | |
| 2960 | ATCTGATTATGGCTA | A572958H | +A*+T*+C*T*G*A*T*T*A*T*G*G*C*T*A*A*+ | 79038 | 19 |
| | AATA | | A*+T*+A | | |
| 2961 | GATCTGATTATGGCT | A572959H | +G*+A*+T*C*T*G*A*T*T*A*T*G*G*C*T*A*+ | 79039 | 19 |
| | AAAT | | A*+A*+T | | |
| 2962 | TGATCTGATTATGGC | A572960H | +T*+G*+A*T*C*T*G*A*T*T*A*T*G*G*C*T*+ | 79040 | 19 |
| | TAAA | | A*+A*+A | | |
| 2963 | CTGATCTGATTATGG | A572961H | +C*+T*+G*A*T*C*T*G*A*T*T*A*T*G*G*C*+ | 79041 | 19 |
| | CTAA | | T*+A*+A | | |
| 2964 | CTGATCTGATTATGG | A572962H | +C*+T*+G*A*T*C*T*G*A*T*T*A*T*G*G*+C* | 79042 | 18 |
| | CTA | | +T*+A | | |
| 2965 | GCTGATCTGATTATG | A572963H | +G*+C*+T*G*A*T*C*T*G*A*T*T*A*T*G*G*+ | 79042 | 19 |
| | GCTA | | C*+T*+A | | |
| 2966 | GCTGATCTGATTATG | A572964H | +G*+C*+T*G*A*T*C*T*G*A*T*T*A*T*G*+G* | 79043 | 18 |
| | GCT | | +C*+T | | |
| 2967 | TGCTGATCTGATTAT | A572965H | +T*+G*+C*T*G*A*T*C*T*G*A*T*T*A*T*G*+ | 79043 | 19 |
| | GGCT | | G*+C*+T | | |
| 2968 | TGCTGATCTGATTAT | A572966H | +T*+G*+C*T*G*A*T*C*T*G*A*T*T*A*T*+G* | 79044 | 18 |
| | GGC | | +G*+C | | |
| 2969 | ATGCTGATCTGATTA | A572967H | +A*+T*+G*C*T*G*A*T*C*T*G*A*T*T*A*T*+ | 79044 | 19 |
| | TGGC | | G*+G*+C | | |
| 2970 | GATGCTGATCTGATT | A572968H | +G*+A*+T*G*C*T*G*A*T*C*T*G*A*T*T*A*+ | 79045 | 19 |
| | ATGG | | T*+G*+G | | |
| 2971 | CAAGATGCTGATCTG | A572969H | +C*+A*+A*G*A*T*G*C*T*G*A*T*C*T*G*A*+ | 79048 | 19 |
| | ATTA | | T*+T*+A | | |
| 2972 | CCAAGATGCTGATCT | A572970H | +C*+C*+A*A*G*A*T*G*C*T*G*A*T*C*T*G*+ | 79049 | 19 |
| | GATT | | A*+T*+T | | |
| 2973 | TCCAAGATGCTGATC | A572971H | +T*+C*+C*A*A*G*A*T*G*C*T*G*A*T*C*T*+ | 79050 | 19 |
| | TGAT | | G*+A*+T | | |
| 2974 | GTCCAAGATGCTGAT | A572972H | +G*+T*+C*C*A*A*G*A*T*G*C*T*G*A*T*C*+ | 79051 | 19 |
| | CTGA | | T*+G*+A | | |
| 2975 | AGTCCAAGATGCTGA | A572973H | +A*+G*+T*C*C*A*A*G*A*T*G*C*T*G*A*T*+ | 79052 | 19 |
| | TCTG | | C*+T*+G | | |
| 2976 | AAGTCCAAGATGCTG | A572974H | +A*+A*+G*T*C*C*A*A*G*A*T*G*C*T*G*A*+ | 79053 | 19 |
| | ATCT | | T*+C*+T | | |
| 2977 | AAGTCCAAGATGCTG | A572975H | +A*+A*+G*T*C*C*A*A*G*A*T*G*C*T*G*+A* | 79054 | 18 |
| | ATC | | +T*+C | | |
| 2978 | CAAGTCCAAGATGCT | A572976H | +C*+A*+A*G*T*C*C*A*A*G*A*T*G*C*T*G*+ | 79054 | 19 |
| | GATC | | A*+T*+C | | |
| 2979 | TCAAGTCCAAGATGC | A572977H | +T*+C*+A*A*G*T*C*C*A*A*G*A*T*G*C*T*+ | 79055 | 19 |
| | TGAT | | G*+A*+T | | |
| 2980 | CTCAAGTCCAAGATG | A572978H | +C*+T*+C*A*A*G*T*C*C*A*A*G*A*T*G*C*+ | 79056 | 19 |
| | CTGA | | T*+G*+A | | |
| 2981 | TTCACTCAAGTCCAA | A572979H | +T*+T*+C*A*C*T*C*A*A*G*T*C*C*A*A*G*+ | 79060 | 19 |
| | GATG | | A*+T*+G | | |
| 2982 | TTCACTCAAGTCCAA | A572980H | +T*+T*+C*A*C*T*C*A*A*G*T*C*C*A*A*+G* | 79061 | 18 |
| | GAT | | +A*+T | | |
| 2983 | TTTCACTCAAGTCCA | A572981H | +T*+T*+T*C*A*C*T*C*A*A*G*T*C*C*A*A*+ | 79061 | 19 |
| | AGAT | | G*+A*+T | | |
| 2984 | TTTTCACTCAAGTCC | A572982H | +T*+T*+T*T*C*A*C*T*C*A*A*G*T*C*C*A*+ | 79062 | 19 |
| | AAGA | | A*+G*+A | | |
| 2985 | GCTTTTTCACTCAAG | A572983H | +G*+C*+T*T*T*T*T*C*A*C*T*C*A*A*G*T*+ | 79065 | 19 |
| | TCCA | | C*+C*+A | | |
| 2986 | CTACTCTAGACCATA | A572984H | +C*+T*+A*C*T*C*T*A*G*A*C*C*A*T*A*A*+ | 79085 | 19 |
| | AATA | | A*+T*+A | | |
| 2987 | CAGTTTCTCTACTCTA | A572985H | +C*+A*+G*T*T*T*C*T*C*T*A*C*T*C*T*A*+ | 79093 | 19 |
| | GAC | | G*+A*+C | | |
| 2988 | TGCAGTTTCTCTACT | A572986H | +T*+G*+C*A*G*T*T*T*C*T*C*T*A*C*T*C*+ | 79095 | 19 |
| | CTAG | | T*+A*+G | | |
| 2989 | ATGCAGTTTCTCTAC | A572987H | +A*+T*+G*C*A*G*T*T*T*C*T*C*T*A*C*T*+ | 79096 | 19 |
| | TCTA | | C*+T*+A | | |
| 2990 | GATGCAGTTTCTCTA | A572988H | +G*+A*+T*G*C*A*G*T*T*T*C*T*C*T*A*C*+ | 79097 | 19 |
| | CTCT | | T*+C*+T | | |
| 2991 | AGATGCAGTTTCTCT | A572989H | +A*+G*+A*T*G*C*A*G*T*T*T*C*T*C*T*A*+ | 79098 | 19 |
| | ACTC | | C*+T*+C | | |
| 2992 | TGTGAGAAAGATGCA | A572990H | +T*+G*+T*G*A*G*A*A*A*G*A*T*G*C*A*G*+ | 79106 | 19 |
| | GTTT | | T*+T*+T | | |
| 2993 | TTGTGAGAAAGATGC | A572991H | +T*+T*+G*T*G*A*G*A*A*A*G*A*T*G*C*A*+ | 79107 | 19 |
| | AGTT | | G*+T*+T | | |
| 2994 | TATTGTGAGAAAGAT | A572992H | +T*+A*+T*T*G*T*G*A*G*A*A*A*G*A*T*G*+ | 79109 | 19 |
| | GCAG | | C*+A*+G | | |
| 2995 | TCAGTTTATTGTGAG | A572993H | +T*+C*+A*G*T*T*T*A*T*T*G*T*G*A*G*A*+ | 79115 | 19 |
| | AAAG | | A*+A*+G | | |
| 2996 | TCTTTCAGTTTATTGT | A572994H | +T*+C*+T*T*T*C*A*G*T*T*T*A*T*T*G*T*+ | 79119 | 19 |
| | GAG | | G*+A*+G | | |
| 2997 | ATGCAAATCATAAAG | A572995Hi | +A*+T*+G*C*A*A*A*T*C*A*T*A*A*A*G*T*+ | 79166 | 19 |
| | TATA | | A*+T*+A | | |
| 2998 | GATGCAAATCATAAA | A572996Hi | +G*+A*+T*G*C*A*A*A*T*C*A*T*A*A*A*G*+ | 79167 | 19 |
| | GTAT | | T*+A*+T | | |
| 2999 | TGATGCAAATCATAA | A572997Hi | +T*+G*+A*T*G*C*A*A*A*T*C*A*T*A*A*A*+ | 79168 | 19 |
| | AGTA | | G*+T*+A | | |
| 3000 | GAAGCTTCTAGTTTC | A572998Hi | +G*+A*+A*G*C*T*T*C*T*A*G*T*T*T*C*A*+ | 79233 | 19 |
| | ATGA | | T*+G*+A | | |
| 3001 | TAGTTAACAGGAAGC | A572999Hi | +T*+A*+G*T*T*A*A*C*A*G*G*A*A*G*C*T*+ | 79243 | 19 |
| | TTCT | | T*+C*+T | | |
| 3002 | TTTATAGTTAACAGG | A573000Hi | +T*+T*+T*A*T*A*G*T*T*A*A*C*A*G*G*A*+ | 79247 | 19 |
| | AAGC | | A*+G*+C | | |
| 3003 | AATTTATAGTTAACA | A573001Hi | +A*+A*+T*T*T*A*T*A*G*T*T*A*A*C*A*G*+ | 79249 | 19 |
| | GGAA | | G*+A*+A | | |
| 3004 | GCTGTTTAGCTTTCA | A573002Hi | +G*+C*+T*G*T*T*T*A*G*C*T*T*T*C*A*G*+ | 79455 | 19 |
| | GATT | | A*+T*+T | | |
| 3005 | AGCTGTTTAGCTTTC | A573003Hi | +A*+G*+C*T*G*T*T*T*A*G*C*T*T*T*C*A*+ | 79456 | 19 |
| | AGAT | | G*+A*+T | | |
| 3006 | TAGCTGTTTAGCTTT | A573004Hi | +T*+A*+G*C*T*G*T*T*T*A*G*C*T*T*T*C*+ | 79457 | 19 |
| | CAGA | | A*+G*+A | | |
| 3007 | TCTAGCTGTTTAGCT | A573005Hi | +T*+C*+T*A*G*C*T*G*T*T*T*A*G*C*T*T*+ | 79459 | 19 |
| | TTCA | | T*+C*+A | | |
| 3008 | ATCTAGCTGTTTAGC | A573006Hi | +A*+T*+C*T*A*G*C*T*G*T*T*T*A*G*C*T*+ | 79460 | 19 |
| | TTTC | | T*+T*+C | | |
| 3009 | TTATATCTAGCTGTTT | A573007Hi | +T*+T*+A*T*A*T*C*T*A*G*C*T*G*T*T*T*+ | 79464 | 19 |
| | AGC | | A*+G*+C | | |
| 3010 | TATATCTAGCTGTTTA | A573008Hi | +T*+A*+T*A*T*C*T*A*G*C*T*G*T*T*T*+A* | 79464 | 18 |
| | GC | | +G*+C | | |
| 3011 | ATTTATATCTAGCTGT | A573009Hi | +A*+T*+T*T*A*T*A*T*C*T*A*G*C*T*G*T*+ | 79466 | 19 |
| | TTA | | T*+T*+A | | |
| 3012 | TACTTTGTACAAATG | A573010Hi | +T*+A*+C*T*T*T*G*T*A*C*A*A*A*T*G*T*+ | 79493 | 19 |
| | TTGG | | T*+G*+G | | |
| 3013 | TTACTTTGTACAAAT | A573011Hi | +T*+T*+A*C*T*T*T*G*T*A*C*A*A*A*T*G*+ | 79494 | 19 |
| | GTTG | | T*+T*+G | | |
| 3014 | TATGTTATATGATAA | A573012Hi | +T*+A*+T*G*T*T*A*T*A*T*G*A*T*A*A*C*+ | 79546 | 19 |
| | CATC | | A*+T*+C | | |
| 3015 | CATAGTTAAGTAACA | A573013Hi | +C*+A*+T*A*G*T*T*A*A*G*T*A*A*C*A*G*+ | 79589 | 19 |
| | GTTA | | T*+T*+A | | |
| 3016 | TTTGTCATAGTTAAG | A573014Hi | +T*+T*+T*G*T*C*A*T*A*G*T*T*A*A*G*T*+ | 79594 | 19 |
| | TAAC | | A*+A*+C | | |
| 3017 | TGAGCTAGTACTATA | A573015Hi | +T*+G*+A*G*C*T*A*G*T*A*C*T*A*T*A*+A* | 79663 | 18 |
| | AAG | | +A*+G | | |
| 3018 | TTATGAGCTAGTACT | A573016Hi | +T*+T*+A*T*G*A*G*C*T*A*G*T*A*C*T*A*+ | 79665 | 19 |
| | ATAA | | T*+A*+A | | |
| 3019 | TTTATGAGCTAGTAC | A573017Hi | +T*+T*+T*A*T*G*A*G*C*T*A*G*T*A*C*T*+ | 79666 | 19 |
| | TATA | | A*+T*+A | | |
| 3020 | TCTTTATGAGCTAGT | A573018Hi | +T*+C*+T*T*T*A*T*G*A*G*C*T*A*G*T*A*+ | 79668 | 19 |
| | ACTA | | C*+T*+A | | |
| 3021 | CACTCTTTATGAGCT | A573019Hi | +C*+A*+C*T*C*T*T*T*A*T*G*A*G*C*T*A*+ | 79671 | 19 |
| | AGTA | | G*+T*+A | | |
| 3022 | CACTCTTTATGAGCT | A573020Hi | +C*+A*+C*T*C*T*T*T*A*T*G*A*G*C*T*+A* | 79672 | 18 |
| | AGT | | +G*+T | | |
| 3023 | ACTATTCCATAGAAC | A573021Hi | +A*+C*+T*A*T*T*C*C*A*T*A*G*A*A*C*A*+ | 79685 | 19 |
| | ACTC | | C*+T*+C | | |
| 3024 | GATATTTACAATGCT | A573022Hi | +G*+A*+T*A*T*T*T*A*C*A*A*T*G*C*T*T*+ | 79735 | 19 |
| | TATT | | A*+T*+T | | |
| 3025 | GATACAATGACACAC | A573023Hi | +G*+A*+T*A*C*A*A*T*G*A*C*A*C*A*C*A*+ | 79910 | 19 |
| | AGCA | | G*+C*+A | | |
| 3026 | TGATACAATGACACA | A573024Hi | +T*+G*+A*T*A*C*A*A*T*G*A*C*A*C*A*C*+ | 79911 | 19 |
| | CAGC | | A*+G*+C | | |
| 3027 | GTTAACAGATTGGAC | A573025Hi | +G*+T*+T*A*A*C*A*G*A*T*T*G*G*A*C*A*+ | 79936 | 19 |
| | AGAG | | G*+A*+G | | |
| 3028 | CTGTTAACAGATTGG | A573026Hi | +C*+T*+G*T*T*A*A*C*A*G*A*T*T*G*G*A*+ | 79938 | 19 |
| | ACAG | | C*+A*+G | | |
| 3029 | TCTGTTAACAGATTG | A573027Hi | +T*+C*+T*G*T*T*A*A*C*A*G*A*T*T*G*G*+ | 79939 | 19 |
| | GACA | | A*+C*+A | | |
| 3030 | GAGTCTGTTAACAGA | A573028Hi | +G*+A*+G*T*C*T*G*T*T*A*A*C*A*G*A*T*+ | 79942 | 19 |
| | TTGG | | T*+G*+G | | |
| 3031 | AGAGTCTGTTAACAG | A573029Hi | +A*+G*+A*G*T*C*T*G*T*T*A*A*C*A*G*A*+ | 79943 | 19 |
| | ATTG | | T*+T*+G | | |
| 3032 | GAGTCTGTTAACAGA | A573030Hi | +G*+A*+G*T*C*T*G*T*T*A*A*C*A*G*A*+T* | 79943 | 18 |
| | TTG | | +T*+G | | |
| 3033 | AAGAGTCTGTTAACA | A573031Hi | +A*+A*+G*A*G*T*C*T*G*T*T*A*A*C*A*G*+ | 79944 | 19 |
| | GATT | | A*+T*+T | | |
| 3034 | AGAAATTTAAGAATG | A573032Hi | +A*+G*+A*A*A*T*T*T*A*A*G*A*A*T*G*T*+ | 80030 | 19 |
| | TACC | | A*+C*+C | | |
| 3035 | CTCACAGTGTCAGAC | A573033Hi | +C*+T*+C*A*C*A*G*T*G*T*C*A*G*A*C*A*+ | 80200 | 19 |
| | ACTT | | C*+T*+T | | |
| 3036 | AAACCTGCATGCAAT | A573034Hi | +A*+A*+A*C*C*T*G*C*A*T*G*C*A*A*T*T*+ | 80385 | 19 |
| | TTAG | | T*+A*+G | | |
| 3037 | ACTACAAACCTGCAT | A573035Hi | +A*+C*+T*A*C*A*A*A*C*C*T*G*C*A*T*G*+ | 80390 | 19 |
| | GCAA | | C*+A*+A | | |
| 3038 | CTGCTCCTTCAGTAA | A573036Hi | +C*+T*+G*C*T*C*C*T*T*C*A*G*T*A*A*+A* | 80424 | 18 |
| | AGG | | +G*+G | | |
| 3039 | CATTCTGCTCCTTCA | A573037Hi | +C*+A*+T*T*C*T*G*C*T*C*C*T*T*C*A*G*+ | 80427 | 19 |
| | GTAA | | T*+A*+A | | |
| 3040 | TCATTCTGCTCCTTCA | A573038Hi | +T*+C*+A*T*T*C*T*G*C*T*C*C*T*T*C*A*+ | 80428 | 19 |
| | GTA | | G*+T*+A | | |
| 3041 | CATTGAAAGTAAGGA | A573039Hi | +C*+A*+T*T*G*A*A*A*G*T*A*A*G*G*A*A*+ | 80487 | 19 |
| | AGTT | | G*+T*+T | | |
| 3042 | CATTGAAAGTAAGGA | A573040Hi | +C*+A*+T*T*G*A*A*A*G*T*A*A*G*G*A*+A* | 80488 | 18 |
| | AGT | | +G*+T | | |
| 3043 | CTTCATCTCTTCAAG | A573041Hi | +C*+T*+T*C*A*T*C*T*C*T*T*C*A*A*G*A*+ | 80503 | 19 |
| | ACAT | | C*+A*+T | | |
| 3044 | ACATACTTCATCTCTT | A573042Hi | +A*+C*+A*T*A*C*T*T*C*A*T*C*T*C*T*T*+ | 80508 | 19 |
| | CAA | | C*+A*+A | | |
| 3045 | ACATACTTCATCTCTT | A573043Hi | +A*+C*+A*T*A*C*T*T*C*A*T*C*T*C*T*+T* | 80509 | 18 |
| | CA | | +C*+A | | |
| 3046 | AGTTGTCAGTATGTG | A573044Hi | +A*+G*+T*T*G*T*C*A*G*T*A*T*G*T*G*+T* | 80616 | 18 |
| | TTT | | +T*+T | | |
| 3047 | TGTCATAAGTTGTCA | A573045Hi | +T*+G*+T*C*A*T*A*A*G*T*T*G*T*C*A*+G* | 80623 | 18 |
| | GTA | | +T*+A | | |
| 3048 | TTTGTCATAAGTTGT | A573046Hi | +T*+T*+T*G*T*C*A*T*A*A*G*T*T*G*T*C*+ | 80624 | 19 |
| | CAGT | | A*+G*+T | | |
| 3049 | TTTGTCATAAGTTGT | A573047Hi | +T*+T*+T*G*T*C*A*T*A*A*G*T*T*G*T*+C* | 80625 | 18 |
| | CAG | | +A*+G | | |
| 3050 | GGTTTGTCATAAGTT | A573048Hi | +G*+G*+T*T*T*G*T*C*A*T*A*A*G*T*T*+G* | 80627 | 18 |
| | GTC | | +T*+C | | |
| 3051 | AAGGTTTGTCATAAG | A573049Hi | +A*+A*+G*G*T*T*T*G*T*C*A*T*A*A*G*+T* | 80629 | 18 |
| | TTG | | +T*+G | | |
| 3052 | TTTCCAAAGGTTTGT | A573050Hi | +T*+T*+T*C*C*A*A*A*G*G*T*T*T*G*T*C*+ | 80634 | 19 |
| | CATA | | A*+T*+A | | |
| 3053 | GATTCCATCAGCAGC | A573051Hi | +G*+A*+T*T*C*C*A*T*C*A*G*C*A*G*C*T*+ | 80679 | 19 |
| | TTGC | | T*+G*+C | | |
| 3054 | GATTCCATCAGCAGC | A573052Hi | +G*+A*+T*T*C*C*A*T*C*A*G*C*A*G*C*+T* | 80680 | 18 |
| | TTG | | +T*+G | | |
| 3055 | AGATTCCATCAGCAG | A573053Hi | +A*+G*+A*T*T*C*C*A*T*C*A*G*C*A*G*C*+ | 80680 | 19 |
| | CTTG | | T*+T*+G | | |
| 3056 | CAGATTCCATCAGCA | A573054Hi | +C*+A*+G*A*T*T*C*C*A*T*C*A*G*C*A*G*+ | 80681 | 19 |
| | GCTT | | C*+T*+T | | |
| 3057 | AGATTCCATCAGCAG | A573055Hi | +A*+G*+A*T*T*C*C*A*T*C*A*G*C*A*G*+C* | 80681 | 18 |
| | CTT | | +T*+T | | |
| 3058 | CACAGATTCCATCAG | A573056Hi | +C*+A*+C*A*G*A*T*T*C*C*A*T*C*A*G*C*+ | 80683 | 19 |
| | CAGC | | A*+G*+C | | |
| 3059 | GCTCACAGATTCCAT | A573057Hi | +G*+C*+T*C*A*C*A*G*A*T*T*C*C*A*T*C*+ | 80686 | 19 |
| | CAGC | | A*+G*+C | | |
| 3060 | GGCTCACAGATTCCA | A573058Hi | +G*+G*+C*T*C*A*C*A*G*A*T*T*C*C*A*T*+ | 80687 | 19 |
| | TCAG | | C*+A*+G | | |
| 3061 | GCTCACAGATTCCAT | A573059Hi | +G*+C*+T*C*A*C*A*G*A*T*T*C*C*A*T*+C* | 80687 | 18 |
| | CAG | | +A*+G | | |
| 3062 | AGAAAGGCTCACAGA | A573060Hi | +A*+G*+A*A*A*G*G*C*T*C*A*C*A*G*A*T*+ | 80692 | 19 |
| | TTCC | | T*+C*+C | | |
| 3063 | AAACAAGAAAGGCTC | A573061Hi | +A*+A*+A*C*A*A*G*A*A*A*G*G*C*T*C*A*+ | 80697 | 19 |
| | ACAG | | C*+A*+G | | |
| 3064 | GAAACAAGAAAGGCT | A573062Hi | +G*+A*+A*A*C*A*A*G*A*A*A*G*G*C*T*C*+ | 80698 | 19 |
| | CACA | | A*+C*+A | | |
| 3065 | AGCCAATCTCAGGAG | A573063H | +A*+G*+C*C*A*A*T*C*T*C*A*G*G*A*G*G*+ | 80789 | 19 |
| | GAAT | | A*+A*+T | | |
| 3066 | CAGCCAATCTCAGGA | A573064H | +C*+A*+G*C*C*A*A*T*C*T*C*A*G*G*A*G*+ | 80790 | 19 |
| | GGAA | | G*+A*+A | | |
| 3067 | AAGACAGCCAATCTC | A573065H | +A*+A*+G*A*C*A*G*C*C*A*A*T*C*T*C*A*+ | 80794 | 19 |
| | AGGA | | G*+G*+A | | |
| 3068 | CAAGACAGCCAATCT | A573066H | +C*+A*+A*G*A*C*A*G*C*C*A*A*T*C*T*C*+ | 80795 | 19 |
| | CAGG | | A*+G*+G | | |
| 3069 | TCAAGACAGCCAATC | A573067H | +T*+C*+A*A*G*A*C*A*G*C*C*A*A*T*C*T*+ | 80796 | 19 |
| | TCAG | | C*+A*+G | | |
| 3070 | TTTCAAGACAGCCAA | A573068H | +T*+T*+T*C*A*A*G*A*C*A*G*C*C*A*A*T*+ | 80798 | 19 |
| | TCTC | | C*+T*+C | | |
| 3071 | GATTTTCAAGACAGC | A573069H | +G*+A*+T*T*T*T*C*A*A*G*A*C*A*G*C*C*+ | 80801 | 19 |
| | CAAT | | A*+A*+T | | |
| 3072 | AGATTTTCAAGACAG | A573070H | +A*+G*+A*T*T*T*T*C*A*A*G*A*C*A*G*C*+ | 80802 | 19 |
| | CCAA | | C*+A*+A | | |
| 3073 | ATGTCAGATTTTCAA | A573071H | +A*+T*+G*T*C*A*G*A*T*T*T*T*C*A*A*G*+ | 80807 | 19 |
| | GACA | | A*+C*+A | | |
| 3074 | GATGTCAGATTTTCA | A573072H | +G*+A*+T*G*T*C*A*G*A*T*T*T*T*C*A*A*+ | 80808 | 19 |
| | AGAC | | G*+A*+C | | |
| 3075 | GAGATGTCAGATTTT | A573073H | +G*+A*+G*A*T*G*T*C*A*G*A*T*T*T*T*C*+ | 80810 | 19 |
| | CAAG | | A*+A*+G | | |
| 3076 | CTGACATCCAGAGAT | A573074H | +C*+T*+G*A*C*A*T*C*C*A*G*A*G*A*T*G*+ | 80820 | 19 |
| | GTCA | | T*+C*+A | | |
| 3077 | ACTGACATCCAGAGA | A573075H | +A*+C*+T*G*A*C*A*T*C*C*A*G*A*G*A*T*+ | 80821 | 19 |
| | TGTC | | G*+T*+C | | |
| 3078 | TTGTAACTGACATCC | A573076H | +T*+T*+G*T*A*A*C*T*G*A*C*A*T*C*C*A*+ | 80826 | 19 |
| | AGAG | | G*+A*+G | | |
| 3079 | GTTGTAACTGACATC | A573077H | +G*+T*+T*G*T*A*A*C*T*G*A*C*A*T*C*C*+ | 80827 | 19 |
| | CAGA | | A*+G*+A | | |
| 3080 | CAAGTTGTAACTGAC | A573078H | +C*+A*+A*G*T*T*G*T*A*A*C*T*G*A*C*A*+ | 80830 | 19 |
| | ATCC | | T*+C*+C | | |
| 3081 | CAAGTTGTAACTGAC | A573079H | +C*+A*+A*G*T*T*G*T*A*A*C*T*G*A*C*+A* | 80831 | 18 |
| | ATC | | +T*+C | | |
| 3082 | CCAAGTTGTAACTGA | A573080H | +C*+C*+A*A*G*T*T*G*T*A*A*C*T*G*A*C*+ | 80831 | 19 |
| | CATC | | A*+T*+C | | |
| 3083 | CCAAGTTGTAACTGA | A573081H | +C*+C*+A*A*G*T*T*G*T*A*A*C*T*G*A*+C* | 80832 | 18 |
| | CAT | | +A*+T | | |
| 3084 | TCCAAGTTGTAACTG | A573082H | +T*+C*+C*A*A*G*T*T*G*T*A*A*C*T*G*A*+ | 80832 | 19 |
| | ACAT | | C*+A*+T | | |
| 3085 | TTCCAAGTTGTAACT | A573083H | +T*+T*+C*C*A*A*G*T*T*G*T*A*A*C*T*G*+ | 80833 | 19 |
| | GACA | | A*+C*+A | | |
| 3086 | GTTCCAAGTTGTAAC | A573084H | +G*+T*+T*C*C*A*A*G*T*T*G*T*A*A*C*T*+ | 80834 | 19 |
| | TGAC | | G*+A*+C | | |
| 3087 | TTAGTTCCAAGTTGT | A573085H | +T*+T*+A*G*T*T*C*C*A*A*G*T*T*G*T*A*+ | 80837 | 19 |
| | AACT | | A*+C*+T | | |
| 3088 | GATCTTAGTTCCAAG | A573086H | +G*+A*+T*C*T*T*A*G*T*T*C*C*A*A*G*T*+ | 80841 | 19 |
| | TTGT | | T*+G*+T | | |
| 3089 | GGATCTTAGTTCCAA | A573087H | +G*+G*+A*T*C*T*T*A*G*T*T*C*C*A*A*G*+ | 80842 | 19 |
| | GTTG | | T*+T*+G | | |
| 3090 | AAGGATCTTAGTTCC | A573088H | +A*+A*+G*G*A*T*C*T*T*A*G*T*T*C*C*A*+ | 80844 | 19 |
| | AAGT | | A*+G*+T | | |
| 3091 | GGGAAAGGATCTTAG | A573089H | +G*+G*+G*A*A*A*G*G*A*T*C*T*T*A*G*T*+ | 80848 | 19 |
| | TTCC | | T*+C*+C | | |
| 3092 | CATTGGGAAAGGATC | A573090H | +C*+A*+T*T*G*G*G*A*A*A*G*G*A*T*C*T*+ | 80852 | 19 |
| | TTAG | | T*+A*+G | | |
| 3093 | TTCATTGGGAAAGGA | A573091H | +T*+T*+C*A*T*T*G*G*G*A*A*A*G*G*A*T*+ | 80854 | 19 |
| | TCTT | | C*+T*+T | | |
| 3094 | TTTCATTGGGAAAGG | A573092H | +T*+T*+T*C*A*T*T*G*G*G*A*A*A*G*G*A*+ | 80855 | 19 |
| | ATCT | | T*+C*+T | | |
| 3095 | TTTCATTGGGAAAGG | A573093H | +T*+T*+T*C*A*T*T*G*G*G*A*A*A*G*G*+A* | 80856 | 18 |
| | ATC | | +T*+C | | |
| 3096 | ATTTCATTGGGAAAG | A573094H | +A*+T*+T*T*C*A*T*T*G*G*G*A*A*A*G*G*+ | 80856 | 19 |
| | GATC | | A*+T*+C | | |
| 3097 | ATATTTTGCTTAATTT | A573095H | +A*+T*+A*T*T*T*T*G*C*T*T*A*A*T*T*T*+ | 80876 | 19 |
| | CCC | | C*+C*+C | | |
| 3098 | ATCCCATATTTTGCTT | A573096H | +A*+T*+C*C*C*A*T*A*T*T*T*T*G*C*T*T*+ | 80881 | 19 |
| | AAT | | A*+A*+T | | |
| 3099 | GGAAGATCCCATATT | A573097H | +G*+G*+A*A*G*A*T*C*C*C*A*T*A*T*T*T*+ | 80886 | 19 |
| | TTGC | | T*+G*+C | | |
| 3100 | CATCCAAAGGAAGAT | A573098H | +C*+A*+T*C*C*A*A*A*G*G*A*A*G*A*T*C*+ | 80894 | 19 |
| | CCCA | | C*+C*+A | | |
| 3101 | TTCATCCAAAGGAAG | A573099H | +T*+T*+C*A*T*C*C*A*A*A*G*G*A*A*G*A*+ | 80896 | 19 |
| | ATCC | | T*+C*+C | | |
| 3102 | GTTCATCCAAAGGAA | A573100H | +G*+T*+T*C*A*T*C*C*A*A*A*G*G*A*A*G*+ | 80897 | 19 |
| | GATC | | A*+T*+C | | |
| 3103 | GCAGTTCATCCAAAG | A573101H | +G*+C*+A*G*T*T*C*A*T*C*C*A*A*A*G*G*+ | 80900 | 19 |
| | GAAG | | A*+A*+G | | |
| 3104 | TACATCCTATATGTTT | A573102H | +T*+A*+C*A*T*C*C*T*A*T*A*T*G*T*T*T*+ | 80933 | 19 |
| | AAA | | A*+A*+A | | |
| 3105 | CTTTGGCTTTACATC | A573103H | +C*+T*+T*T*G*G*C*T*T*T*A*C*A*T*C*C*+ | 80942 | 19 |
| | CTAT | | T*+A*+T | | |
| 3106 | TCTTTGGCTTTACATC | A573104H | +T*+C*+T*T*T*G*G*C*T*T*T*A*C*A*T*C*+ | 80943 | 19 |
| | CTA | | C*+T*+A | | |
| 3107 | ATGTCTTTGGCTTTA | A573105H | +A*+T*+G*T*C*T*T*T*G*G*C*T*T*T*A*C*+ | 80946 | 19 |
| | CATC | | A*+T*+C | | |
| 3108 | TGATGTCTTTGGCTT | A573106H | +T*+G*+A*T*G*T*C*T*T*T*G*G*C*T*T*T*+ | 80948 | 19 |
| | TACA | | A*+C*+A | | |
| 3109 | ATGATGTCTTTGGCT | A573107H | +A*+T*+G*A*T*G*T*C*T*T*T*G*G*C*T*T*+ | 80949 | 19 |
| | TTAC | | T*+A*+C | | |
| 3110 | GTCCATCAAAGTCAC | A573108Hi | +G*+T*+C*C*A*T*C*A*A*A*G*T*C*A*C*+A* | 81026 | 18 |
| | AGA | | +G*+A | | |
| 3111 | CTCTACAGTGGCACC | A573109Hi | +C*+T*+C*T*A*C*A*G*T*G*G*C*A*C*C*A*+ | 81134 | 19 |
| | ACAG | | C*+A*+G | | |
| 3112 | TCTCTACAGTGGCAC | A573110Hi | +T*+C*+T*C*T*A*C*A*G*T*G*G*C*A*C*C*+ | 81135 | 19 |
| | CACA | | A*+C*+A | | |
| 3113 | TTTAAGATGTAAGAG | A573111Hi | +T*+T*+T*A*A*G*A*T*G*T*A*A*G*A*G*G*+ | 81345 | 19 |
| | GCAT | | C*+A*+T | | |
| 3114 | GAGTAGATTACTCAT | A573112Hi | +G*+A*+G*T*A*G*A*T*T*A*C*T*C*A*T*+C* | 81416 | 18 |
| | CCT | | +C*+T | | |
| 3115 | ACTGATCAAGGTGAG | A573113Hi | +A*+C*+T*G*A*T*C*A*A*G*G*T*G*A*G*T*+ | 81427 | 19 |
| | TAGA | | A*+G*+A | | |
| 3116 | ACTGATCAAGGTGAG | A573114Hi | +A*+C*+T*G*A*T*C*A*A*G*G*T*G*A*G*+T* | 81428 | 18 |
| | TAG | | +A*+G | | |
| 3117 | ACAGTGATACTTGAG | A573115Hi | +A*+C*+A*G*T*G*A*T*A*C*T*T*G*A*G*A*+ | 81461 | 19 |
| | AAGT | | A*+G*+T | | |
| 3118 | CAGTGATACTTGAGA | A573116Hi | +C*+A*+G*T*G*A*T*A*C*T*T*G*A*G*A*+A* | 81461 | 18 |
| | AGT | | +G*+T | | |
| 3119 | AACAGTGATACTTGA | A573117Hi | +A*+A*+C*A*G*T*G*A*T*A*C*T*T*G*A*G*+ | 81462 | 19 |
| | GAAG | | A*+A*+G | | |
| 3120 | AGACCAGAAACAGTG | A573118Hi | +A*+G*+A*C*C*A*G*A*A*A*C*A*G*T*G*A*+ | 81470 | 19 |
| | ATAC | | T*+A*+C | | |
| 3121 | AAGACCAGAAACAGT | A573119Hi | +A*+A*+G*A*C*C*A*G*A*A*A*C*A*G*T*G*+ | 81471 | 19 |
| | GATA | | A*+T*+A | | |
| 3122 | AAAGACCAGAAACAG | A573120Hi | +A*+A*+A*G*A*C*C*A*G*A*A*A*C*A*G*T*+ | 81472 | 19 |
| | TGAT | | G*+A*+T | | |
| 3123 | GTAAAGACCAGAAAC | A573121Hi | +G*+T*+A*A*A*G*A*C*C*A*G*A*A*A*C*A*+ | 81474 | 19 |
| | AGTG | | G*+T*+G | | |
| 3124 | TGAGAAAGTAATAAG | A573122Hi | +T*+G*+A*G*A*A*A*G*T*A*A*T*A*A*G*+A* | 81560 | 18 |
| | ACC | | +C*+C | | |
| 3125 | ATGAGAAAGTAATAA | A573123Hi | +A*+T*+G*A*G*A*A*A*G*T*A*A*T*A*A*G*+ | 81560 | 19 |
| | GACC | | A*+C*+C | | |
| 3126 | TTTATGAGAAAGTAA | A573124Hi | +T*+T*+T*A*T*G*A*G*A*A*A*G*T*A*A*T*+ | 81563 | 19 |
| | TAAG | | A*+A*+G | | |
| 3127 | TTACAGTCAAGTTGA | A573125Hi | +T*+T*+A*C*A*G*T*C*A*A*G*T*T*G*A*G*+ | 81643 | 19 |
| | GAGG | | A*+G*+G | | |
| 3128 | GATATTACAGTCAAG | A573126Hi | +G*+A*+T*A*T*T*A*C*A*G*T*C*A*A*G*T*+ | 81647 | 19 |
| | TTGA | | T*+G*+A | | |
| 3129 | AATCACTGGTAATTA | A573127Hi | +A*+A*+T*C*A*C*T*G*G*T*A*A*T*T*A*A*+ | 81748 | 19 |
| | AACA | | A*+C*+A | | |
| 3130 | CTACTAAATGTTTAG | A573128Hi | +C*+T*+A*C*T*A*A*A*T*G*T*T*T*A*G*T*+ | 81984 | 19 |
| | TAAG | | A*+A*+G | | |
| 3131 | CTAGTTCTACTAAAT | A573129Hi | +C*+T*+A*G*T*T*C*T*A*C*T*A*A*A*T*G*+ | 81990 | 19 |
| | GTTT | | T*+T*+T | | |
| 3132 | GCTAGTTCTACTAAA | A573130Hi | +G*+C*+T*A*G*T*T*C*T*A*C*T*A*A*A*T*+ | 81991 | 19 |
| | TGTT | | G*+T*+T | | |
| 3133 | ATAAACTATTATACTA | A573131Hi | +A*+T*+A*A*A*C*T*A*T*T*A*T*A*C*T*A*+ | 82514 | 19 |
| | TCA | | T*+C*+A | | |
| 3134 | CAAAGTCCATATGAC | A573132Hi | +C*+A*+A*A*G*T*C*C*A*T*A*T*G*A*C*A*+ | 82717 | 19 |
| | ATAG | | T*+A*+G | | |
| 3135 | CACAAAGTCCATATG | A573133Hi | +C*+A*+C*A*A*A*G*T*C*C*A*T*A*T*G*+A* | 82720 | 18 |
| | ACA | | +C*+A | | |
| 3136 | TCACAAAGTCCATAT | A573134Hi | +T*+C*+A*C*A*A*A*G*T*C*C*A*T*A*T*G*+ | 82720 | 19 |
| | GACA | | A*+C*+A | | |
| 3137 | TAGAATACGATATGC | A573135Hi | +T*+A*+G*A*A*T*A*C*G*A*T*A*T*G*C*A*+ | 83172 | 19 |
| | AATA | | A*+T*+A | | |
| 3138 | TAGAATACGATATGC | A573136Hi | +T*+A*+G*A*A*T*A*C*G*A*T*A*T*G*C*+A* | 83173 | 18 |
| | AAT | | +A*+T | | |
| 3139 | TTAGAATACGATATG | A573137Hi | +T*+T*+A*G*A*A*T*A*C*G*A*T*A*T*G*+C* | 83174 | 18 |
| | CAA | | +A*+A | | |
| 3140 | TAATTAGAATACGAT | A573138Hi | +T*+A*+A*T*T*A*G*A*A*T*A*C*G*A*T*+A* | 83177 | 18 |
| | ATG | | +T*+G | | |
| 3141 | CTAATTAGAATACGA | A573139Hi | +C*+T*+A*A*T*T*A*G*A*A*T*A*C*G*A*T*+ | 83177 | 19 |
| | TATG | | A*+T*+G | | |
| 3142 | CTAATTAGAATACGA | A573140Hi | +C*+T*+A*A*T*T*A*G*A*A*T*A*C*G*A*+T* | 83178 | 18 |
| | TAT | | +A*+T | | |
| 3143 | ACTAATTAGAATACG | A573141Hi | +A*+C*+T*A*A*T*T*A*G*A*A*T*A*C*G*A*+ | 83178 | 19 |
| | ATAT | | T*+A*+T | | |
| 3144 | GTACTGTTAATGCAT | A573142Hi | +G*+T*+A*C*T*G*T*T*A*A*T*G*C*A*T*A*+ | 83302 | 19 |
| | ATAT | | T*+A*+T | | |
| 3145 | TGTACATGTCTTTCA | A573143Hi | +T*+G*+T*A*C*A*T*G*T*C*T*T*T*C*A*G*+ | 83319 | 19 |
| | GAGT | | A*+G*+T | | |
| 3146 | ATGTACATGTCTTTC | A573144Hi | +A*+T*+G*T*A*C*A*T*G*T*C*T*T*T*C*A*+ | 83320 | 19 |
| | AGAG | | G*+A*+G | | |
| 3147 | AATAATGTACATGTC | A573145Hi | +A*+A*+T*A*A*T*G*T*A*C*A*T*G*T*C*T*+ | 83324 | 19 |
| | TTTC | | T*+T*+C | | |
| 3148 | AGATTTAGGTGAGTT | A573146Hi | +A*+G*+A*T*T*T*A*G*G*T*G*A*G*T*T*C*+ | 83400 | 19 |
| | CTTT | | T*+T*+T | | |
| 3149 | AAGTTTCATTCGGTT | A573147H | +A*+A*+G*T*T*T*C*A*T*T*C*G*G*T*T*+A* | 83505 | 18 |
| | ATA | | +T*+A | | |
| 3150 | TAAGTTTCATTCGGT | A573148H | +T*+A*+A*G*T*T*T*C*A*T*T*C*G*G*T*T*+ | 83505 | 19 |
| | TATA | | A*+T*+A | | |
| 3151 | ATAAGTTTCATTCGG | A573149H | +A*+T*+A*A*G*T*T*T*C*A*T*T*C*G*G*T*+ | 83506 | 19 |
| | TTAT | | T*+A*+T | | |
| 3152 | ATAAGTTTCATTCGG | A573150H | +A*+T*+A*A*G*T*T*T*C*A*T*T*C*G*G*+T* | 83507 | 18 |
| | TTA | | +T*+A | | |
| 3153 | CATAAGTTTCATTCG | A573151H | +C*+A*+T*A*A*G*T*T*T*C*A*T*T*C*G*G*+ | 83507 | 19 |
| | GTTA | | T*+T*+A | | |
| 3154 | TTTACCACTCCCAGT | A573152H | +T*+T*+T*A*C*C*A*C*T*C*C*C*A*G*T*A*+ | 83534 | 19 |
| | ATTT | | T*+T*+T | | |
| 3155 | TTTACCACTCCCAGT | A573153H | +T*+T*+T*A*C*C*A*C*T*C*C*C*A*G*T*+A* | 83535 | 18 |
| | ATT | | +T*+T | | |
| 3156 | TTTTACCACTCCCAG | A573154H | +T*+T*+T*T*A*C*C*A*C*T*C*C*C*A*G*T*+ | 83535 | 19 |
| | TATT | | A*+T*+T | | |
| 3157 | GTTTTACCACTCCCA | A573155H | +G*+T*+T*T*T*A*C*C*A*C*T*C*C*C*A*G*+ | 83536 | 19 |
| | GTAT | | T*+A*+T | | |
| 3158 | GTTTTACCACTCCCA | A573156H | +G*+T*+T*T*T*A*C*C*A*C*T*C*C*C*A*+G* | 83537 | 18 |
| | GTA | | +T*+A | | |
| 3159 | GGTTTTACCACTCCC | A573157H | +G*+G*+T*T*T*T*A*C*C*A*C*T*C*C*C*A*+ | 83537 | 19 |
| | AGTA | | G*+T*+A | | |
| 3160 | TGGTTTTACCACTCC | A573158H | +T*+G*+G*T*T*T*T*A*C*C*A*C*T*C*C*C*+ | 83538 | 19 |
| | CAGT | | A*+G*+T | | |
| 3161 | GTGGTTTTACCACTC | A573159H | +G*+T*+G*G*T*T*T*T*A*C*C*A*C*T*C*C*+ | 83539 | 19 |
| | CCAG | | C*+A*+G | | |
| 3162 | GGTGGTTTTACCACT | A573160H | +G*+G*+T*G*G*T*T*T*T*A*C*C*A*C*T*C*+ | 83540 | 19 |
| | CCCA | | C*+C*+A | | |
| 3163 | GGTGGTTTTACCACT | A573161H | +G*+G*+T*G*G*T*T*T*T*A*C*C*A*C*T*+C* | 83541 | 18 |
| | CCC | | +C*+C | | |
| 3164 | AGGTGGTTTTACCAC | A573162H | +A*+G*+G*T*G*G*T*T*T*T*A*C*C*A*C*T*+ | 83541 | 19 |
| | TCCC | | C*+C*+C | | |
| 3165 | AGGTGGTTTTACCAC | A573163H | +A*+G*+G*T*G*G*T*T*T*T*A*C*C*A*C*+T* | 83542 | 18 |
| | TCC | | +C*+C | | |
| 3166 | AAGGTGGTTTTACCA | A573164H | +A*+A*+G*G*T*G*G*T*T*T*T*A*C*C*A*C*+ | 83542 | 19 |
| | CTCC | | T*+C*+C | | |
| 3167 | CTTGGTTTTCATTAAT | A573165H | +C*+T*+T*G*G*T*T*T*T*C*A*T*T*A*A*T*+ | 83567 | 19 |
| | TGC | | T*+G*+C | | |
| 3168 | TCTTGGTTTTCATTAA | A573166H | +T*+C*+T*T*G*G*T*T*T*T*C*A*T*T*A*A*+ | 83568 | 19 |
| | TTG | | T*+T*+G | | |
| 3169 | CAAGATCTGATTTCT | A573167H | +C*+A*+A*G*A*T*C*T*G*A*T*T*T*C*T*T*+ | 83580 | 19 |
| | TGGT | | G*+G*+T | | |
| 3170 | TGCATTCCAAGATCT | A573168H | +T*+G*+C*A*T*T*C*C*A*A*G*A*T*C*T*G*+ | 83587 | 19 |
| | GATT | | A*+T*+T | | |
| 3171 | ACTTTGCATTCCAAG | A573169H | +A*+C*+T*T*T*G*C*A*T*T*C*C*A*A*G*A*+ | 83591 | 19 |
| | ATCT | | T*+C*+T | | |
| 3172 | GCACTTTGCATTCCA | A573170H | +G*+C*+A*C*T*T*T*G*C*A*T*T*C*C*A*A*+ | 83593 | 19 |
| | AGAT | | G*+A*+T | | |
| 3173 | GGCACTTTGCATTCC | A573171H | +G*+G*+C*A*C*T*T*T*G*C*A*T*T*C*C*A*+ | 83594 | 19 |
| | AAGA | | A*+G*+A | | |
| 3174 | GGCACTTTGCATTCC | A573172H | +G*+G*+C*A*C*T*T*T*G*C*A*T*T*C*C*+A* | 83595 | 18 |
| | AAG | | +A*+G | | |
| 3175 | TGGCACTTTGCATTC | A573173H | +T*+G*+G*C*A*C*T*T*T*G*C*A*T*T*C*C*+ | 83595 | 19 |
| | CAAG | | A*+A*+G | | |
| 3176 | TGCCAACTGTGGCAC | A573174H | +T*+G*+C*C*A*A*C*T*G*T*G*G*C*A*C*T*+ | 83604 | 19 |
| | TTTG | | T*+T*+G | | |
| 3177 | ATGCCAACTGTGGCA | A573175H | +A*+T*+G*C*C*A*A*C*T*G*T*G*G*C*A*C*+ | 83605 | 19 |
| | CTTT | | T*+T*+T | | |
| 3178 | TATGCCAACTGTGGC | A573176H | +T*+A*+T*G*C*C*A*A*C*T*G*T*G*G*C*A*+ | 83606 | 19 |
| | ACTT | | C*+T*+T | | |
| 3179 | CTATGCCAACTGTGG | A573177H | +C*+T*+A*T*G*C*C*A*A*C*T*G*T*G*G*C*+ | 83607 | 19 |
| | CACT | | A*+C*+T | | |
| 3180 | CTATGCCAACTGTGG | A573178H | +C*+T*+A*T*G*C*C*A*A*C*T*G*T*G*G*+C* | 83608 | 18 |
| | CAC | | +A*+C | | |
| 3181 | TCTATGCCAACTGTG | A573179H | +T*+C*+T*A*T*G*C*C*A*A*C*T*G*T*G*G*+ | 83608 | 19 |
| | GCAC | | C*+A*+C | | |
| 3182 | ATCTATGCCAACTGT | A573180H | +A*+T*+C*T*A*T*G*C*C*A*A*C*T*G*T*G*+ | 83609 | 19 |
| | GGCA | | G*+C*+A | | |
| 3183 | CATCTATGCCAACTG | A573181H | +C*+A*+T*C*T*A*T*G*C*C*A*A*C*T*G*T*+ | 83610 | 19 |
| | TGGC | | G*+G*+C | | |
| 3184 | CATCTATGCCAACTG | A573182H | +C*+A*+T*C*T*A*T*G*C*C*A*A*C*T*G*+T* | 83611 | 18 |
| | TGG | | +G*+G | | |
| 3185 | ACATCTATGCCAACT | A573183H | +A*+C*+A*T*C*T*A*T*G*C*C*A*A*C*T*G*+ | 83611 | 19 |
| | GTGG | | T*+G*+G | | |
| 3186 | CACATCTATGCCAAC | A573184H | +C*+A*+C*A*T*C*T*A*T*G*C*C*A*A*C*T*+ | 83612 | 19 |
| | TGTG | | G*+T*+G | | |
| 3187 | CACATCTATGCCAAC | A573185H | +C*+A*+C*A*T*C*T*A*T*G*C*C*A*A*C*+T* | 83613 | 18 |
| | TGT | | +G*+T | | |
| 3188 | TCACATCTATGCCAA | A573186H | +T*+C*+A*C*A*T*C*T*A*T*G*C*C*A*A*C*+ | 83613 | 19 |
| | CTGT | | T*+G*+T | | |
| 3189 | TTCACATCTATGCCA | A573187H | +T*+T*+C*A*C*A*T*C*T*A*T*G*C*C*A*A*+ | 83614 | 19 |
| | ACTG | | C*+T*+G | | |
| 3190 | CCAGTCTTTCACATC | A573188H | +C*+C*+A*G*T*C*T*T*T*C*A*C*A*T*C*T*+ | 83621 | 19 |
| | TATG | | A*+T*+G | | |
| 3191 | CCAGTCTTTCACATC | A573189H | +C*+C*+A*G*T*C*T*T*T*C*A*C*A*T*C*+T* | 83622 | 18 |
| | TAT | | +A*+T | | |
| 3192 | GCCAGTCTTTCACAT | A573190H | +G*+C*+C*A*G*T*C*T*T*T*C*A*C*A*T*C*+ | 83622 | 19 |
| | CTAT | | T*+A*+T | | |
| 3193 | GGCCAGTCTTTCACA | A573191H | +G*+G*+C*C*A*G*T*C*T*T*T*C*A*C*A*T*+ | 83623 | 19 |
| | TCTA | | C*+T*+A | | |
| 3194 | AGGCCAGTCTTTCAC | A573192H | +A*+G*+G*C*C*A*G*T*C*T*T*T*C*A*C*A*+ | 83624 | 19 |
| | ATCT | | T*+C*+T | | |
| 3195 | TGGATAGGCCAGTCT | A573193H | +T*+G*+G*A*T*A*G*G*C*C*A*G*T*C*T*T*+ | 83629 | 19 |
| | TTCA | | T*+C*+A | | |
| 3196 | TTGGATAGGCCAGTC | A573194H | +T*+T*+G*G*A*T*A*G*G*C*C*A*G*T*C*T*+ | 83630 | 19 |
| | TTTC | | T*+T*+C | | |
| 3197 | TTGGATAGGCCAGTC | A573195H | +T*+T*+G*G*A*T*A*G*G*C*C*A*G*T*C*+T* | 83631 | 18 |
| | TTT | | +T*+T | | |
| 3198 | TTTGGATAGGCCAGT | A573196H | +T*+T*+T*G*G*A*T*A*G*G*C*C*A*G*T*C*+ | 83631 | 19 |
| | CTTT | | T*+T*+T | | |
| 3199 | ATTTGGATAGGCCAG | A573197H | +A*+T*+T*T*G*G*A*T*A*G*G*C*C*A*G*T*+ | 83632 | 19 |
| | TCTT | | C*+T*+T | | |
| 3200 | ATTTGGATAGGCCAG | A573198H | +A*+T*+T*T*G*G*A*T*A*G*G*C*C*A*G*+T* | 83633 | 18 |
| | TCT | | +C*+T | | |
| 3201 | TATTTGGATAGGCCA | A573199H | +T*+A*+T*T*T*G*G*A*T*A*G*G*C*C*A*G*+ | 83633 | 19 |
| | GTCT | | T*+C*+T | | |
| 3202 | TTATTTGGATAGGCC | A573200H | +T*+T*+A*T*T*T*G*G*A*T*A*G*G*C*C*A*+ | 83634 | 19 |
| | AGTC | | G*+T*+C | | |
| 3203 | TTATTTGGATAGGCC | A573201H | +T*+T*+A*T*T*T*G*G*A*T*A*G*G*C*C*+A* | 83635 | 18 |
| | AGT | | +G*+T | | |
| 3204 | CTTATTTGGATAGGC | A573202H | +C*+T*+T*A*T*T*T*G*G*A*T*A*G*G*C*C*+ | 83635 | 19 |
| | CAGT | | A*+G*+T | | |
| 3205 | CTTATTTGGATAGGC | A573203H | +C*+T*+T*A*T*T*T*G*G*A*T*A*G*G*C*+C* | 83636 | 18 |
| | CAG | | +A*+G | | |
| 3206 | TCTTATTTGGATAGG | A573204H | +T*+C*+T*T*A*T*T*T*G*G*A*T*A*G*G*C*+ | 83636 | 19 |
| | CCAG | | C*+A*+G | | |
| 3207 | TCTTATTTGGATAGG | A573205H | +T*+C*+T*T*A*T*T*T*G*G*A*T*A*G*G*+C* | 83637 | 18 |
| | CCA | | +C*+A | | |
| 3208 | CTCTTATTTGGATAG | A573206H | +C*+T*+C*T*T*A*T*T*T*G*G*A*T*A*G*G*+ | 83637 | 19 |
| | GCCA | | C*+C*+A | | |
| 3209 | CTCTTATTTGGATAG | A573207H | +C*+T*+C*T*T*A*T*T*T*G*G*A*T*A*G*+G* | 83638 | 18 |
| | GCC | | +C*+C | | |
| 3210 | ACGAGATCTCTCTTT | A573208H | +A*+C*+G*A*G*A*T*C*T*C*T*C*T*T*T*C*+ | 83659 | 19 |
| | CTTT | | T*+T*+T | | |
| 3211 | CTGCAAAATCCCACA | A573209H | +C*+T*+G*C*A*A*A*A*T*C*C*C*A*C*A*C*+ | 83682 | 19 |
| | CATT | | A*+T*+T | | |
| 3212 | GTTTCTGGATATTAG | A573210Hi | +G*+T*+T*T*C*T*G*G*A*T*A*T*T*A*G*A*+ | 83775 | 19 |
| | AACA | | A*+C*+A | | |
| 3213 | CAGGTTTCTGGATAT | A573211Hi | +C*+A*+G*G*T*T*T*C*T*G*G*A*T*A*T*T*+ | 83778 | 19 |
| | TAGA | | A*+G*+A | | |
| 3214 | CAGGTTTCTGGATAT | A573212Hi | +C*+A*+G*G*T*T*T*C*T*G*G*A*T*A*T*+T* | 83779 | 18 |
| | TAG | | +A*+G | | |
| 3215 | TATTTAGCATACTAAT | A573213Hi | +T*+A*+T*T*T*A*G*C*A*T*A*C*T*A*A*T*+ | 83997 | 19 |
| | AAG | | A*+A*+G | | |
| 3216 | CGCTGCGTCATAAAA | A573214H | +C*+G*+C*T*G*C*G*T*C*A*T*A*A*A*+A*+T | 84139 | 17 |
| | TG | | *+G | | |
| 3217 | TCGCTGCGTCATAAA | A573215H | +T*+C*+G*C*T*G*C*G*T*C*A*T*A*A*A*+A* | 84139 | 18 |
| | ATG | | +T*+G | | |
| 3218 | TCGCTGCGTCATAAA | A573216H | +T*+C*+G*C*T*G*C*G*T*C*A*T*A*A*+A*+A | 84140 | 17 |
| | AT | | *+T | | |
| 3219 | GCTCGCTGCGTCATA | A573217H | +G*+C*+T*C*G*C*T*G*C*G*T*C*A*T*A*A*+ | 84140 | 19 |
| | AAAT | | A*+A*+T | | |
| 3220 | TCGCTGCGTCATAAA | A573218H | +T*+C*+G*C*T*G*C*G*T*C*A*T*A*+A*+A*+ | 84141 | 16 |
| | A | | A | | |
| 3221 | GCTCGCTGCGTCATA | A573219H | +G*+C*+T*C*G*C*T*G*C*G*T*C*A*T*A*+A* | 84141 | 18 |
| | AAA | | +A*+A | | |
| 3222 | TGCTCGCTGCGTCAT | A573220H | +T*+G*+C*T*C*G*C*T*G*C*G*T*C*A*T*A*+ | 84141 | 19 |
| | AAAA | | A*+A*+A | | |
| 3223 | GCTCGCTGCGTCATA | A573221H | +G*+C*+T*C*G*C*T*G*C*G*T*C*A*T*+A*+A | 84142 | 17 |
| | AA | | *+A | | |
| 3224 | TGCTCGCTGCGTCAT | A573222H | +T*+G*+C*T*C*G*C*T*G*C*G*T*C*A*T*+A* | 84142 | 18 |
| | AAA | | +A*+A | | |
| 3225 | ATGCTCGCTGCGTCA | A573223H | +A*+T*+G*C*T*C*G*C*T*G*C*G*T*C*A*T*+ | 84142 | 19 |
| | TAAA | | A*+A*+A | | |
| 3226 | GCTCGCTGCGTCATA | A573224H | +G*+C*+T*C*G*C*T*G*C*G*T*C*A*+T*+A*+ | 84143 | 16 |
| | A | | A | | |
| 3227 | TGCTCGCTGCGTCAT | A573225H | +T*+G*+C*T*C*G*C*T*G*C*G*T*C*A*+T*+A | 84143 | 17 |
| | AA | | *+A | | |
| 3228 | ATGCTCGCTGCGTCA | A573226H | +A*+T*+G*C*T*C*G*C*T*G*C*G*T*C*A*+T* | 84143 | 18 |
| | TAA | | +A*+A | | |
| 3229 | AATGCTCGCTGCGTC | A573227H | +A*+A*+T*G*C*T*C*G*C*T*G*C*G*T*C*A*+ | 84143 | 19 |
| | ATAA | | T*+A*+A | | |
| 3230 | AATGCTCGCTGCGTC | A573228H | +A*+A*+T*G*C*T*C*G*C*T*G*C*G*T*C*+A* | 84144 | 18 |
| | ATA | | +T*+A | | |
| 3231 | CAATGCTCGCTGCGT | A573229H | +C*+A*+A*T*G*C*T*C*G*C*T*G*C*G*T*C*+ | 84144 | 19 |
| | CATA | | A*+T*+A | | |
| 3232 | AATGCTCGCTGCGTC | A573230H | +A*+A*+T*G*C*T*C*G*C*T*G*C*G*T*+C*+A | 84145 | 17 |
| | AT | | *+T | | |
| 3233 | CAATGCTCGCTGCGT | A573231H | +C*+A*+A*T*G*C*T*C*G*C*T*G*C*G*T*+C* | 84145 | 18 |
| | CAT | | +A*+T | | |
| 3234 | ACAATGCTCGCTGCG | A573232H | +A*+C*+A*A*T*G*C*T*C*G*C*T*G*C*G*T*+ | 84145 | 19 |
| | TCAT | | C*+A*+T | | |
| 3235 | CAATGCTCGCTGCGT | A573233H | +C*+A*+A*T*G*C*T*C*G*C*T*G*C*G*+T*+C | 84146 | 17 |
| | CA | | *+A | | |
| 3236 | ACAATGCTCGCTGCG | A573234H | +A*+C*+A*A*T*G*C*T*C*G*C*T*G*C*G*+T* | 84146 | 18 |
| | TCA | | +C*+A | | |
| 3237 | TACAATGCTCGCTGC | A573235H | +T*+A*+C*A*A*T*G*C*T*C*G*C*T*G*C*G*+ | 84146 | 19 |
| | GTCA | | T*+C*+A | | |
| 3238 | TACAATGCTCGCTGC | A573236H | +T*+A*+C*A*A*T*G*C*T*C*G*C*T*G*C*+G* | 84147 | 18 |
| | GTC | | +T*+C | | |
| 3239 | GTACAATGCTCGCTG | A573237H | +G*+T*+A*C*A*A*T*G*C*T*C*G*C*T*G*C*+ | 84147 | 19 |
| | CGTC | | G*+T*+C | | |
| 3240 | TACAATGCTCGCTGC | A573238H | +T*+A*+C*A*A*T*G*C*T*C*G*C*T*G*+C*+G | 84148 | 17 |
| | GT | | *+T | | |
| 3241 | GTACAATGCTCGCTG | A573239H | +G*+T*+A*C*A*A*T*G*C*T*C*G*C*T*G*+C* | 84148 | 18 |
| | CGT | | +G*+T | | |
| 3242 | GGTACAATGCTCGCT | A573240H | +G*+G*+T*A*C*A*A*T*G*C*T*C*G*C*T*G*+ | 84148 | 19 |
| | GCGT | | C*+G*+T | | |
| 3243 | TACAATGCTCGCTGC | A573241H | +T*+A*+C*A*A*T*G*C*T*C*G*C*T*+G*+C*+ | 84149 | 16 |
| | G | | G | | |
| 3244 | GTACAATGCTCGCTG | A573242H | +G*+T*+A*C*A*A*T*G*C*T*C*G*C*T*+G*+C | 84149 | 17 |
| | CG | | *+G | | |
| 3245 | GGTACAATGCTCGCT | A573243H | +G*+G*+T*A*C*A*A*T*G*C*T*C*G*C*T*+G* | 84149 | 18 |
| | GCG | | +C*+G | | |
| 3246 | AGGTACAATGCTCGC | A573244H | +A*+G*+G*T*A*C*A*A*T*G*C*T*C*G*C*T*+ | 84149 | 19 |
| | TGCG | | G*+C*+G | | |
| 3247 | AGGTACAATGCTCGC | A573245H | +A*+G*+G*T*A*C*A*A*T*G*C*T*C*G*C*+T* | 84150 | 18 |
| | TGC | | +G*+C | | |
| 3248 | AAGGTACAATGCTCG | A573246H | +A*+A*+G*G*T*A*C*A*A*T*G*C*T*C*G*C*+ | 84150 | 19 |
| | CTGC | | T*+G*+C | | |
| 3249 | AGGTACAATGCTCGC | A573247H | +A*+G*+G*T*A*C*A*A*T*G*C*T*C*G*+C*+T | 84151 | 17 |
| | TG | | *+G | | |
| 3250 | AAGGTACAATGCTCG | A573248H | +A*+A*+G*G*T*A*C*A*A*T*G*C*T*C*G*+C* | 84151 | 18 |
| | CTG | | +T*+G | | |
| 3251 | CAAGGTACAATGCTC | A573249H | +C*+A*+A*G*G*T*A*C*A*A*T*G*C*T*C*G*+ | 84151 | 19 |
| | GCTG | | C*+T*+G | | |
| 3252 | AAGGTACAATGCTCG | A573250H | +A*+A*+G*G*T*A*C*A*A*T*G*C*T*C*+G*+C | 84152 | 17 |
| | CT | | *+T | | |
| 3253 | CAAGGTACAATGCTC | A573251H | +C*+A*+A*G*G*T*A*C*A*A*T*G*C*T*C*+G* | 84152 | 18 |
| | GCT | | +C*+T | | |
| 3254 | GCAAGGTACAATGCT | A573252H | +G*+C*+A*A*G*G*T*A*C*A*A*T*G*C*T*C*+ | 84152 | 19 |
| | CGCT | | G*+C*+T | | |
| 3255 | AGCAAGGTACAATGC | A573253H | +A*+G*+C*A*A*G*G*T*A*C*A*A*T*G*C*T*+ | 84153 | 19 |
| | TCGC | | C*+G*+C | | |
| 3256 | AGCAAGGTACAATGC | A573254H | +A*+G*+C*A*A*G*G*T*A*C*A*A*T*G*C*+T* | 84154 | 18 |
| | TCG | | +C*+G | | |
| 3257 | CAGCAAGGTACAATG | A573255H | +C*+A*+G*C*A*A*G*G*T*A*C*A*A*T*G*C*+ | 84154 | 19 |
| | CTCG | | T*+C*+G | | |
| 3258 | ACAGCAAGGTACAAT | A573256H | +A*+C*+A*G*C*A*A*G*G*T*A*C*A*A*T*G*+ | 84155 | 19 |
| | GCTC | | C*+T*+C | | |
| 3259 | GACAGCAAGGTACAA | A573257H | +G*+A*+C*A*G*C*A*A*G*G*T*A*C*A*A*T*+ | 84156 | 19 |
| | TGCT | | G*+C*+T | | |
| 3260 | AGACAGCAAGGTACA | A573258H | +A*+G*+A*C*A*G*C*A*A*G*G*T*A*C*A*A*+ | 84157 | 19 |
| | ATGC | | T*+G*+C | | |
| 3261 | TAGACAGCAAGGTAC | A573259H | +T*+A*+G*A*C*A*G*C*A*A*G*G*T*A*C*A*+ | 84158 | 19 |
| | AATG | | A*+T*+G | | |
| 3262 | GTCATAGACAGCAAG | A573260H | +G*+T*+C*A*T*A*G*A*C*A*G*C*A*A*G*G*+ | 84162 | 19 |
| | GTAC | | T*+A*+C | | |
| 3263 | GGTCATAGACAGCAA | A573261H | +G*+G*+T*C*A*T*A*G*A*C*A*G*C*A*A*G*+ | 84163 | 19 |
| | GGTA | | G*+T*+A | | |
| 3264 | GGTCATAGACAGCAA | A573262H | +G*+G*+T*C*A*T*A*G*A*C*A*G*C*A*A*+G* | 84164 | 18 |
| | GGT | | +G*+T | | |
| 3265 | AGGTCATAGACAGCA | A573263H | +A*+G*+G*T*C*A*T*A*G*A*C*A*G*C*A*A*+ | 84164 | 19 |
| | AGGT | | G*+G*+T | | |
| 3266 | AGGTCATAGACAGCA | A573264H | +A*+G*+G*T*C*A*T*A*G*A*C*A*G*C*A*+A* | 84165 | 18 |
| | AGG | | +G*+G | | |
| 3267 | ATCAACTTCAGCCTG | A573265H | +A*+T*+C*A*A*C*T*T*C*A*G*C*C*T*G*+T* | 84190 | 18 |
| | TCC | | +C*+C | | |
| 3268 | CATCAACTTCAGCCT | A573266H | +C*+A*+T*C*A*A*C*T*T*C*A*G*C*C*T*G*+ | 84190 | 19 |
| | GTCC | | T*+C*+C | | |
| 3269 | GCATCAACTTCAGCC | A573267H | +G*+C*+A*T*C*A*A*C*T*T*C*A*G*C*C*T*+ | 84191 | 19 |
| | TGTC | | G*+T*+C | | |
| 3270 | GCATCAACTTCAGCC | A573268H | +G*+C*+A*T*C*A*A*C*T*T*C*A*G*C*C*+T* | 84192 | 18 |
| | TGT | | +G*+T | | |
| 3271 | GGCATCAACTTCAGC | A573269H | +G*+G*+C*A*T*C*A*A*C*T*T*C*A*G*C*C*+ | 84192 | 19 |
| | CTGT | | T*+G*+T | | |
| 3272 | GGCATCAACTTCAGC | A573270H | +G*+G*+C*A*T*C*A*A*C*T*T*C*A*G*C*+C* | 84193 | 18 |
| | CTG | | +T*+G | | |
| 3273 | TGGCATCAACTTCAG | A573271H | +T*+G*+G*C*A*T*C*A*A*C*T*T*C*A*G*C*+ | 84193 | 19 |
| | CCTG | | C*+T*+G | | |
| 3274 | TGGCATCAACTTCAG | A573272H | +T*+G*+G*C*A*T*C*A*A*C*T*T*C*A*G*+C* | 84194 | 18 |
| | CCT | | +C*+T | | |
| 3275 | ATGGCATCAACTTCA | A573273H | +A*+T*+G*G*C*A*T*C*A*A*C*T*T*C*A*G*+ | 84194 | 19 |
| | GCCT | | C*+C*+T | | |
| 3276 | CATGGCATCAACTTC | A573274H | +C*+A*+T*G*G*C*A*T*C*A*A*C*T*T*C*A*+ | 84195 | 19 |
| | AGCC | | G*+C*+C | | |
| 3277 | TCATGGCATCAACTT | A573275H | +T*+C*+A*T*G*G*C*A*T*C*A*A*C*T*T*C*+ | 84196 | 19 |
| | CAGC | | A*+G*+C | | |
| 3278 | TTCATGGCATCAACT | A573276H | +T*+T*+C*A*T*G*G*C*A*T*C*A*A*C*T*T*+ | 84197 | 19 |
| | TCAG | | C*+A*+G | | |
| 3279 | AGGCTTCATGGCATC | A573277H | +A*+G*+G*C*T*T*C*A*T*G*G*C*A*T*C*A*+ | 84201 | 19 |
| | AACT | | A*+C*+T | | |
| 3280 | AAGGCTTCATGGCAT | A573278H | +A*+A*+G*G*C*T*T*C*A*T*G*G*C*A*T*C*+ | 84202 | 19 |
| | CAAC | | A*+A*+C | | |
| 3281 | AGCCAAGGCTTCATG | A573279H | +A*+G*+C*C*A*A*G*G*C*T*T*C*A*T*G*G*+ | 84206 | 19 |
| | GCAT | | C*+A*+T | | |
| 3282 | AGCCAAGGCTTCATG | A573280H | +A*+G*+C*C*A*A*G*G*C*T*T*C*A*T*G*+G* | 84207 | 18 |
| | GCA | | +C*+A | | |
| 3283 | GAGCCAAGGCTTCAT | A573281H | +G*+A*+G*C*C*A*A*G*G*C*T*T*C*A*T*G*+ | 84207 | 19 |
| | GGCA | | G*+C*+A | | |
| 3284 | AGAGCCAAGGCTTCA | A573282H | +A*+G*+A*G*C*C*A*A*G*G*C*T*T*C*A*T*+ | 84208 | 19 |
| | TGGC | | G*+G*+C | | |
| 3285 | AAGAGCCAAGGCTTC | A573283H | +A*+A*+G*A*G*C*C*A*A*G*G*C*T*T*C*A*+ | 84209 | 19 |
| | ATGG | | T*+G*+G | | |
| 3286 | GAAGAGCCAAGGCTT | A573284H | +G*+A*+A*G*A*G*C*C*A*A*G*G*C*T*T*C*+ | 84210 | 19 |
| | CATG | | A*+T*+G | | |
| 3287 | GAAGAGCCAAGGCTT | A573285H | +G*+A*+A*G*A*G*C*C*A*A*G*G*C*T*T*+C* | 84211 | 18 |
| | CAT | | +A*+T | | |
| 3288 | TGAAGAGCCAAGGCT | A573286H | +T*+G*+A*A*G*A*G*C*C*A*A*G*G*C*T*T*+ | 84211 | 19 |
| | TCAT | | C*+A*+T | | |
| 3289 | TATTGAAGAGCCAAG | A573287H | +T*+A*+T*T*G*A*A*G*A*G*C*C*A*A*G*G*+ | 84214 | 19 |
| | GCTT | | C*+T*+T | | |
| 3290 | TTATATTGAAGAGCC | A573288H | +T*+T*+A*T*A*T*T*G*A*A*G*A*G*C*C*A*+ | 84217 | 19 |
| | AAGG | | A*+G*+G | | |
| 3291 | TTATATTGAAGAGCC | A573289H | +T*+T*+A*T*A*T*T*G*A*A*G*A*G*C*C*+A* | 84218 | 18 |
| | AAG | | +A*+G | | |
| 3292 | TTTATATTGAAGAGC | A573290H | +T*+T*+T*A*T*A*T*T*G*A*A*G*A*G*C*C*+ | 84218 | 19 |
| | CAAG | | A*+A*+G | | |
| 3293 | CTTTATATTGAAGAG | A573291H | +C*+T*+T*T*A*T*A*T*T*G*A*A*G*A*G*C*+ | 84219 | 19 |
| | CCAA | | C*+A*+A | | |
| 3294 | GACCACTTAAAGGAT | A573292Hi | +G*+A*+C*C*A*C*T*T*A*A*A*G*G*A*T*A*+ | 84643 | 19 |
| | ACAA | | C*+A*+A | | |
| 3295 | ATAGCTGACAACAAG | A573293Hi | +A*+T*+A*G*C*T*G*A*C*A*A*C*A*A*G*A*+ | 84724 | 19 |
| | ACAA | | C*+A*+A | | |
| 3296 | GAAGAGTTCATTGCA | A573294Hi | +G*+A*+A*G*A*G*T*T*C*A*T*T*G*C*A*G*+ | 84795 | 19 |
| | GCAT | | C*+A*+T | | |
| 3297 | AGAAGAGTTCATTGC | A573295Hi | +A*+G*+A*A*G*A*G*T*T*C*A*T*T*G*C*A*+ | 84796 | 19 |
| | AGCA | | G*+C*+A | | |
| 3298 | CAGAAGAGTTCATTG | A573296Hi | +C*+A*+G*A*A*G*A*G*T*T*C*A*T*T*G*C*+ | 84797 | 19 |
| | CAGC | | A*+G*+C | | |
| 3299 | GAGTCTACAATGAAA | A573297Hi | +G*+A*+G*T*C*T*A*C*A*A*T*G*A*A*A*G*+ | 84987 | 19 |
| | GAAT | | A*+A*+T | | |
| 3300 | TATATCAAATTAACG | A573298Hi | +T*+A*+T*A*T*C*A*A*A*T*T*A*A*C*G*T*+ | 85076 | 19 |
| | TCAT | | C*+A*+T | | |
| 3301 | TTATATCAAATTAAC | A573299Hi | +T*+T*+A*T*A*T*C*A*A*A*T*T*A*A*C*G*+ | 85077 | 19 |
| | GTCA | | T*+C*+A | | |
| 3302 | TATATCAAATTAACG | A573300Hi | +T*+A*+T*A*T*C*A*A*A*T*T*A*A*C*G*+T* | 85077 | 18 |
| | TCA | | +C*+A | | |
| 3303 | AAATTATATCAAATTA | A573301Hi | +A*+A*+A*T*T*A*T*A*T*C*A*A*A*T*T*A*+ | 85080 | 19 |
| | ACG | | A*+C*+G | | |
| 3304 | GCTTTCCGTGACATT | A573302Hi | +G*+C*+T*T*T*C*C*G*T*G*A*C*A*T*T*+C* | 85345 | 18 |
| | CAC | | +A*+C | | |
| 3305 | TTGCTTTCCGTGACA | A573303Hi | +T*+T*+G*C*T*T*T*C*C*G*T*G*A*C*A*T*+ | 85346 | 19 |
| | TTCA | | T*+C*+A | | |
| 3306 | TGCTTTCCGTGACAT | A573304Hi | +T*+G*+C*T*T*T*C*C*G*T*G*A*C*A*T*+T* | 85346 | 18 |
| | TCA | | +C*+A | | |
| 3307 | TTGCTTTCCGTGACA | A573305Hi | +T*+T*+G*C*T*T*T*C*C*G*T*G*A*C*A*+T* | 85347 | 18 |
| | TTC | | +T*+C | | |
| 3308 | ATTTGCTTTCCGTGA | A573306Hi | +A*+T*+T*T*G*C*T*T*T*C*C*G*T*G*A*C*+ | 85348 | 19 |
| | CATT | | A*+T*+T | | |
| 3309 | GTTGATATTTGCTTTC | A573307Hi | +G*+T*+T*G*A*T*A*T*T*T*G*C*T*T*T*C*+ | 85354 | 19 |
| | CGT | | C*+G*+T | | |
| 3310 | CTCACATTCCTGTTG | A573308Hi | +C*+T*+C*A*C*A*T*T*C*C*T*G*T*T*G*A*+ | 85365 | 19 |
| | ATAT | | T*+A*+T | | |
| 3311 | CTCACATTCCTGTTG | A573309Hi | +C*+T*+C*A*C*A*T*T*C*C*T*G*T*T*G*+A* | 85366 | 18 |
| | ATA | | +T*+A | | |
| 3312 | GCTCACATTCCTGTT | A573310Hi | +G*+C*+T*C*A*C*A*T*T*C*C*T*G*T*T*+G* | 85367 | 18 |
| | GAT | | +A*+T | | |
| 3313 | GGCCTGCTCACATTC | A573311Hi | +G*+G*+C*C*T*G*C*T*C*A*C*A*T*T*C*C*+ | 85371 | 19 |
| | CTGT | | T*+G*+T | | |
| 3314 | GGAAGAAGAAGCGCG | A573312H | +G*+G*+A*A*G*A*A*G*A*A*G*C*G*C*G*+A | 85435 | 18 |
| | AGC | | *+G*+C | | |
| 3315 | GGGAAGAAGAAGCGC | A573313H | +G*+G*+G*A*A*G*A*A*G*A*A*G*C*G*C*G* | 85435 | 19 |
| | GAGC | | +A*+G*+C | | |
| 3316 | GGGAAGAAGAAGCGC | A573314H | +G*+G*+G*A*A*G*A*A*G*A*A*G*C*G*C*+G | 85436 | 18 |
| | GAG | | *+A*+G | | |
| 3317 | GGGGAAGAAGAAGC | A573315H | +G*+G*+G*G*A*A*G*A*A*G*A*A*G*C*G*C* | 85436 | 19 |
| | GCGAG | | +G*+A*+G | | |
| 3318 | GGGAAGAAGAAGCGC | A573316H | +G*+G*+G*A*A*G*A*A*G*A*A*G*C*G*+C*+ | 85437 | 17 |
| | GA | | G*+A | | |
| 3319 | GGGGAAGAAGAAGC | A573317H | +G*+G*+G*G*A*A*G*A*A*G*A*A*G*C*G*+C | 85437 | 18 |
| | GCGA | | *+G*+A | | |
| 3320 | AGGGGAAGAAGAAGC | A573318H | +A*+G*+G*G*G*A*A*G*A*A*G*A*A*G*C*G* | 85437 | 19 |
| | GCGA | | +C*+G*+A | | |
| 3321 | ACAGGGGAAGAAGAA | A573319H | +A*+C*+A*G*G*G*G*A*A*G*A*A*G*A*A*G* | 85439 | 19 |
| | GCGC | | +C*+G*+C | | |
| 3322 | TCACAGGGGAAGAAG | A573320H | +T*+C*+A*C*A*G*G*G*G*A*A*G*A*A*G*A* | 85441 | 19 |
| | AAGC | | +A*+G*+C | | |
| 3323 | AATCACAGGGGAAGA | A573321H | +A*+A*+T*C*A*C*A*G*G*G*G*A*A*G*A*A* | 85443 | 19 |
| | AGAA | | +G*+A*+A | | |
| 3324 | AACGAGAATCACAGG | A573322H | +A*+A*+C*G*A*G*A*A*T*C*A*C*A*G*G*G* | 85449 | 19 |
| | GGAA | | +G*+A*+A | | |
| 3325 | GTGCCAACGAGAATC | A573323H | +G*+T*+G*C*C*A*A*C*G*A*G*A*A*T*C*A*+ | 85454 | 19 |
| | ACAG | | C*+A*+G | | |
| 3326 | GTGCCAACGAGAATC | A573324H | +G*+T*+G*C*C*A*A*C*G*A*G*A*A*T*C*+A* | 85455 | 18 |
| | ACA | | +C*+A | | |
| 3327 | TGTGCCAACGAGAAT | A573325H | +T*+G*+T*G*C*C*A*A*C*G*A*G*A*A*T*C*+ | 85455 | 19 |
| | CACA | | A*+C*+A | | |
| 3328 | GTGTGCCAACGAGAA | A573326H | +G*+T*+G*T*G*C*C*A*A*C*G*A*G*A*A*T*+ | 85456 | 19 |
| | TCAC | | C*+A*+C | | |
| 3329 | GTGTGCCAACGAGAA | A573327H | +G*+T*+G*T*G*C*C*A*A*C*G*A*G*A*A*+T* | 85457 | 18 |
| | TCA | | +C*+A | | |
| 3330 | TGTGTGCCAACGAGA | A573328H | +T*+G*+T*G*T*G*C*C*A*A*C*G*A*G*A*A*+ | 85457 | 19 |
| | ATCA | | T*+C*+A | | |
| 3331 | AATGTGTGCCAACGA | A573329H | +A*+A*+T*G*T*G*T*G*C*C*A*A*C*G*A*G*+ | 85459 | 19 |
| | GAAT | | A*+A*+T | | |
| 3332 | AAATGTGTGCCAACG | A573330H | +A*+A*+A*T*G*T*G*T*G*C*C*A*A*C*G*A*+ | 85460 | 19 |
| | AGAA | | G*+A*+A | | |
| 3333 | AAATGTGTGCCAACG | A573331H | +A*+A*+A*T*G*T*G*T*G*C*C*A*A*C*G*+A* | 85461 | 18 |
| | AGA | | +G*+A | | |
| 3334 | CAAATGTGTGCCAAC | A573332H | +C*+A*+A*A*T*G*T*G*T*G*C*C*A*A*C*G*+ | 85461 | 19 |
| | GAGA | | A*+G*+A | | |
| 3335 | CAAATGTGTGCCAAC | A573333H | +C*+A*+A*A*T*G*T*G*T*G*C*C*A*A*C*+G* | 85462 | 18 |
| | GAG | | +A*+G | | |
| 3336 | CCAAATGTGTGCCAA | A573334H | +C*+C*+A*A*A*T*G*T*G*T*G*C*C*A*A*C*+ | 85462 | 19 |
| | CGAG | | G*+A*+G | | |
| 3337 | CCAAATGTGTGCCAA | A573335H | +C*+C*+A*A*A*T*G*T*G*T*G*C*C*A*A*+C* | 85463 | 18 |
| | CGA | | +G*+A | | |
| 3338 | TCCAAATGTGTGCCA | A573336H | +T*+C*+C*A*A*A*T*G*T*G*T*G*C*C*A*A*+ | 85463 | 19 |
| | ACGA | | C*+G*+A | | |
| 3339 | TCCAAATGTGTGCCA | A573337H | +T*+C*+C*A*A*A*T*G*T*G*T*G*C*C*A*+A* | 85464 | 18 |
| | ACG | | +C*+G | | |
| 3340 | ATCCAAATGTGTGCC | A573338H | +A*+T*+C*C*A*A*A*T*G*T*G*T*G*C*C*A*+ | 85464 | 19 |
| | AACG | | A*+C*+G | | |
| 3341 | AGTTCCTTGGTGATT | A573339H | +A*+G*+T*T*C*C*T*T*G*G*T*G*A*T*T*T*+ | 85517 | 19 |
| | TTAC | | T*+A*+C | | |
| 3342 | GGAGTTCCTTGGTGA | A573340H | +G*+G*+A*G*T*T*C*C*T*T*G*G*T*G*A*T*+ | 85519 | 19 |
| | TTTT | | T*+T*+T | | |
| 3343 | TCAGGAGTTCCTTGG | A573341H | +T*+C*+A*G*G*A*G*T*T*C*C*T*T*G*G*T*+ | 85522 | 19 |
| | TGAT | | G*+A*+T | | |
| 3344 | ATTCAGGAGTTCCTT | A573342H | +A*+T*+T*C*A*G*G*A*G*T*T*C*C*T*T*G*+ | 85524 | 19 |
| | GGTG | | G*+T*+G | | |
| 3345 | GCTTATTCAGGAGTT | A573343H | +G*+C*+T*T*A*T*T*C*A*G*G*A*G*T*T*C*+ | 85528 | 19 |
| | CCTT | | C*+T*+T | | |
| 3346 | CGCTTATTCAGGAGT | A573344H | +C*+G*+C*T*T*A*T*T*C*A*G*G*A*G*T*T*+ | 85529 | 19 |
| | TCCT | | C*+C*+T | | |
| 3347 | CGCTTATTCAGGAGT | A573345H | +C*+G*+C*T*T*A*T*T*C*A*G*G*A*G*T*+T* | 85530 | 18 |
| | TCC | | +C*+C | | |
| 3348 | TCGCTTATTCAGGAG | A573346H | +T*+C*+G*C*T*T*A*T*T*C*A*G*G*A*G*T*+ | 85530 | 19 |
| | TTCC | | T*+C*+C | | |
| 3349 | CCTCGCTTATTCAGG | A573347H | +C*+C*+T*C*G*C*T*T*A*T*T*C*A*G*G*A*+ | 85532 | 19 |
| | AGTT | | G*+T*+T | | |
| 3350 | CCTCGCTTATTCAGG | A573348H | +C*+C*+T*C*G*C*T*T*A*T*T*C*A*G*G*+A* | 85533 | 18 |
| | AGT | | +G*+T | | |
| 3351 | CCCTCGCTTATTCAG | A573349H | +C*+C*+C*T*C*G*C*T*T*A*T*T*C*A*G*G*+ | 85533 | 19 |
| | GAGT | | A*+G*+T | | |
| 3352 | ACCCTCGCTTATTCA | A573350H | +A*+C*+C*C*T*C*G*C*T*T*A*T*T*C*A*G*+ | 85534 | 19 |
| | GGAG | | G*+A*+G | | |
| 3353 | AACCCTCGCTTATTC | A573351H | +A*+A*+C*C*C*T*C*G*C*T*T*A*T*T*C*A*+ | 85535 | 19 |
| | AGGA | | G*+G*+A | | |
| 3354 | AACCCTCGCTTATTC | A573352H | +A*+A*+C*C*C*T*C*G*C*T*T*A*T*T*C*+A* | 85536 | 18 |
| | AGG | | +G*+G | | |
| 3355 | GAACCCTCGCTTATT | A573353H | +G*+A*+A*C*C*C*T*C*G*C*T*T*A*T*T*C*+ | 85536 | 19 |
| | CAGG | | A*+G*+G | | |
| 3356 | GAACCCTCGCTTATT | A573354H | +G*+A*+A*C*C*C*T*C*G*C*T*T*A*T*T*+C* | 85537 | 18 |
| | CAG | | +A*+G | | |
| 3357 | GGAACCCTCGCTTAT | A573355H | +G*+G*+A*A*C*C*C*T*C*G*C*T*T*A*T*T*+ | 85537 | 19 |
| | TCAG | | C*+A*+G | | |
| 3358 | GGAACCCTCGCTTAT | A573356H | +G*+G*+A*A*C*C*C*T*C*G*C*T*T*A*T*+T* | 85538 | 18 |
| | TCA | | +C*+A | | |
| 3359 | GGGAACCCTCGCTTA | A573357H | +G*+G*+G*A*A*C*C*C*T*C*G*C*T*T*A*T*+ | 85538 | 19 |
| | TTCA | | T*+C*+A | | |
| 3360 | GGAACCCTCGCTTAT | A573358H | +G*+G*+A*A*C*C*C*T*C*G*C*T*T*A*+T*+T | 85539 | 17 |
| | TC | | *+C | | |
| 3361 | GGGAACCCTCGCTTA | A573359H | +G*+G*+G*A*A*C*C*C*T*C*G*C*T*T*A*+T* | 85539 | 18 |
| | TTC | | +T*+C | | |
| 3362 | AGGGAACCCTCGCTT | A573360H | +A*+G*+G*G*A*A*C*C*C*T*C*G*C*T*T*A*+ | 85539 | 19 |
| | ATTC | | T*+T*+C | | |
| 3363 | GGGAACCCTCGCTTA | A573361H | +G*+G*+G*A*A*C*C*C*T*C*G*C*T*T*+A*+T | 85540 | 17 |
| | TT | | *+T | | |
| 3364 | AGGGAACCCTCGCTT | A573362H | +A*+G*+G*G*A*A*C*C*C*T*C*G*C*T*T*+A* | 85540 | 18 |
| | ATT | | +T*+T | | |
| 3365 | CAGGGAACCCTCGCT | A573363H | +C*+A*+G*G*G*A*A*C*C*C*T*C*G*C*T*T*+ | 85540 | 19 |
| | TATT | | A*+T*+T | | |
| 3366 | GGGAACCCTCGCTTA | A573364H | +G*+G*+G*A*A*C*C*C*T*C*G*C*T*+T*+A*+ | 85541 | 16 |
| | T | | T | | |
| 3367 | AGGGAACCCTCGCTT | A573365H | +A*+G*+G*G*A*A*C*C*C*T*C*G*C*T*+T*+A | 85541 | 17 |
| | AT | | *+T | | |
| 3368 | CAGGGAACCCTCGCT | A573366H | +C*+A*+G*G*G*A*A*C*C*C*T*C*G*C*T*+T* | 85541 | 18 |
| | TAT | | +A*+T | | |
| 3369 | GCAGGGAACCCTCGC | A573367H | +G*+C*+A*G*G*G*A*A*C*C*C*T*C*G*C*T*+ | 85541 | 19 |
| | TTAT | | T*+A*+T | | |
| 3370 | GCAGGGAACCCTCGC | A573368H | +G*+C*+A*G*G*G*A*A*C*C*C*T*C*G*C*+T* | 85542 | 18 |
| | TTA | | +T*+A | | |
| 3371 | AAAGTGGTAATCTCG | A573369H | +A*+A*+A*G*T*G*G*T*A*A*T*C*T*C*G*+T* | 85561 | 18 |
| | TAT | | +A*+T | | |
| 3372 | CAAAGTGGTAATCTC | A573370H | +C*+A*+A*A*G*T*G*G*T*A*A*T*C*T*C*G*+ | 85561 | 19 |
| | GTAT | | T*+A*+T | | |
| 3373 | CAAAGTGGTAATCTC | A573371H | +C*+A*+A*A*G*T*G*G*T*A*A*T*C*T*C*+G* | 85562 | 18 |
| | GTA | | +T*+A | | |
| 3374 | ACAAAGTGGTAATCT | A573372H | +A*+C*+A*A*A*G*T*G*G*T*A*A*T*C*T*C*+ | 85562 | 19 |
| | CGTA | | G*+T*+A | | |
| 3375 | ACAAAGTGGTAATCT | A573373H | +A*+C*+A*A*A*G*T*G*G*T*A*A*T*C*T*+C* | 85563 | 18 |
| | CGT | | +G*+T | | |
| 3376 | CACAAAGTGGTAATC | A573374H | +C*+A*+C*A*A*A*G*T*G*G*T*A*A*T*C*T*+ | 85563 | 19 |
| | TCGT | | C*+G*+T | | |
| 3377 | TCACAAAGTGGTAAT | A573375H | +T*+C*+A*C*A*A*A*G*T*G*G*T*A*A*T*C*+ | 85564 | 19 |
| | CTCG | | T*+C*+G | | |
| 3378 | TTCACAAAGTGGTAA | A573376H | +T*+T*+C*A*C*A*A*A*G*T*G*G*T*A*A*T*+ | 85565 | 19 |
| | TCTC | | C*+T*+C | | |
| 3379 | CATTCACAAAGTGGT | A573377H | +C*+A*+T*T*C*A*C*A*A*A*G*T*G*G*T*A*+ | 85567 | 19 |
| | AATC | | A*+T*+C | | |
| 3380 | GGCATTCACAAAGTG | A573378H | +G*+G*+C*A*T*T*C*A*C*A*A*A*G*T*G*G*+ | 85569 | 19 |
| | GTAA | | T*+A*+A | | |
| 3381 | TGGCATTCACAAAGT | A573379H | +T*+G*+G*C*A*T*T*C*A*C*A*A*A*G*T*G*+ | 85570 | 19 |
| | GGTA | | G*+T*+A | | |
| 3382 | GGTGGCATTCACAAA | A573380H | +G*+G*+T*G*G*C*A*T*T*C*A*C*A*A*A*G*+ | 85572 | 19 |
| | GTGG | | T*+G*+G | | |
| 3383 | CGGTGGCATTCACAA | A573381H | +C*+G*+G*T*G*G*C*A*T*T*C*A*C*A*A*A*+ | 85573 | 19 |
| | AGTG | | G*+T*+G | | |
| 3384 | CTCGGTGGCATTCAC | A573382H | +C*+T*+C*G*G*T*G*G*C*A*T*T*C*A*C*A*+ | 85575 | 19 |
| | AAAG | | A*+A*+G | | |
| 3385 | CTCGGTGGCATTCAC | A573383H | +C*+T*+C*G*G*T*G*G*C*A*T*T*C*A*C*+A* | 85576 | 18 |
| | AAA | | +A*+A | | |
| 3386 | CCTCGGTGGCATTCA | A573384H | +C*+C*+T*C*G*G*T*G*G*C*A*T*T*C*A*C*+ | 85576 | 19 |
| | CAAA | | A*+A*+A | | |
| 3387 | TCCTCGGTGGCATTC | A573385H | +T*+C*+C*T*C*G*G*T*G*G*C*A*T*T*C*A*+ | 85577 | 19 |
| | ACAA | | C*+A*+A | | |
| 3388 | ATTCCTCGGTGGCAT | A573386H | +A*+T*+T*C*C*T*C*G*G*T*G*G*C*A*T*T*+ | 85579 | 19 |
| | TCAC | | C*+A*+C | | |
| 3389 | AGATTCCTCGGTGGC | A573387H | +A*+G*+A*T*T*C*C*T*C*G*G*T*G*G*C*A*+ | 85581 | 19 |
| | ATTC | | T*+T*+C | | |
| 3390 | CAGATTCCTCGGTGG | A573388H | +C*+A*+G*A*T*T*C*C*T*C*G*G*T*G*G*C*+ | 85582 | 19 |
| | CATT | | A*+T*+T | | |
| 3391 | CATCAGATTCCTCGG | A573389H | +C*+A*+T*C*A*G*A*T*T*C*C*T*C*G*G*T*+ | 85585 | 19 |
| | TGGC | | G*+G*+C | | |
| 3392 | CATCAGATTCCTCGG | A573390H | +C*+A*+T*C*A*G*A*T*T*C*C*T*C*G*G*+T* | 85586 | 18 |
| | TGG | | +G*+G | | |
| 3393 | GCATCAGATTCCTCG | A573391H | +G*+C*+A*T*C*A*G*A*T*T*C*C*T*C*G*G*+ | 85586 | 19 |
| | GTGG | | T*+G*+G | | |
| 3394 | GCATCAGATTCCTCG | A573392H | +G*+C*+A*T*C*A*G*A*T*T*C*C*T*C*G*+G* | 85587 | 18 |
| | GTG | | +T*+G | | |
| 3395 | AGCATCAGATTCCTC | A573393H | +A*+G*+C*A*T*C*A*G*A*T*T*C*C*T*C*G*+ | 85587 | 19 |
| | GGTG | | G*+T*+G | | |
| 3396 | AAGCATCAGATTCCT | A573394H | +A*+A*+G*C*A*T*C*A*G*A*T*T*C*C*T*C*+ | 85588 | 19 |
| | CGGT | | G*+G*+T | | |
| 3397 | AAAGCATCAGATTCC | A573395H | +A*+A*+A*G*C*A*T*C*A*G*A*T*T*C*C*T*+ | 85589 | 19 |
| | TCGG | | C*+G*+G | | |
| 3398 | GCCAAAGCATCAGAT | A573396H | +G*+C*+C*A*A*A*G*C*A*T*C*A*G*A*T*T*+ | 85592 | 19 |
| | TCCT | | C*+C*+T | | |
| 3399 | TGCCAAAGCATCAGA | A573397H | +T*+G*+C*C*A*A*A*G*C*A*T*C*A*G*A*T*+ | 85593 | 19 |
| | TTCC | | T*+C*+C | | |
| 3400 | GTTTTGCCAAAGCAT | A573398H | +G*+T*+T*T*T*G*C*C*A*A*A*G*C*A*T*C*+ | 85597 | 19 |
| | CAGA | | A*+G*+A | | |
| 3401 | GTTTTGCCAAAGCAT | A573399H | +G*+T*+T*T*T*G*C*C*A*A*A*G*C*A*T*+C* | 85598 | 18 |
| | CAG | | +A*+G | | |
| 3402 | AGTTTTGCCAAAGCA | A573400H | +A*+G*+T*T*T*T*G*C*C*A*A*A*G*C*A*T*+ | 85598 | 19 |
| | TCAG | | C*+A*+G | | |
| 3403 | GAAGTTTTGCCAAAG | A573401H | +G*+A*+A*G*T*T*T*T*G*C*C*A*A*A*G*C*+ | 85600 | 19 |
| | CATC | | A*+T*+C | | |
| 3404 | CGAAGTTTTGCCAAA | A573402H | +C*+G*+A*A*G*T*T*T*T*G*C*C*A*A*A*G*+ | 85601 | 19 |
| | GCAT | | C*+A*+T | | |
| 3405 | CCGAAGTTTTGCCAA | A573403H | +C*+C*+G*A*A*G*T*T*T*T*G*C*C*A*A*A*+ | 85602 | 19 |
| | AGCA | | G*+C*+A | | |
| 3406 | CCGAAGTTTTGCCAA | A573404H | +C*+C*+G*A*A*G*T*T*T*T*G*C*C*A*A*+A* | 85603 | 18 |
| | AGC | | +G*+C | | |
| 3407 | TCCGAAGTTTTGCCA | A573405H | +T*+C*+C*G*A*A*G*T*T*T*T*G*C*C*A*A*+ | 85603 | 19 |
| | AAGC | | A*+G*+C | | |
| 3408 | TTCCGAAGTTTTGCC | A573406H | +T*+T*+C*C*G*A*A*G*T*T*T*T*G*C*C*A*+ | 85604 | 19 |
| | AAAG | | A*+A*+G | | |
| 3409 | TTTCCGAAGTTTTGC | A573407H | +T*+T*+T*C*C*G*A*A*G*T*T*T*T*G*C*C*+ | 85605 | 19 |
| | CAAA | | A*+A*+A | | |
| 3410 | TTTTCCGAAGTTTTG | A573408H | +T*+T*+T*T*C*C*G*A*A*G*T*T*T*T*G*C*+ | 85606 | 19 |
| | CCAA | | C*+A*+A | | |
| 3411 | TTTTCCGAAGTTTTG | A573409H | +T*+T*+T*T*C*C*G*A*A*G*T*T*T*T*G*+C* | 85607 | 18 |
| | CCA | | +C*+A | | |
| 3412 | GTTTTCCGAAGTTTT | A573410H | +G*+T*+T*T*T*C*C*G*A*A*G*T*T*T*T*G*+ | 85607 | 19 |
| | GCCA | | C*+C*+A | | |
| 3413 | GGTTTTCCGAAGTTT | A573411H | +G*+G*+T*T*T*T*C*C*G*A*A*G*T*T*T*T*+ | 85608 | 19 |
| | TGCC | | G*+C*+C | | |
| 3414 | ATGGTTTTCCGAAGT | A573412H | +A*+T*+G*G*T*T*T*T*C*C*G*A*A*G*T*T*+ | 85610 | 19 |
| | TTTG | | T*+T*+G | | |
| 3415 | GATGGTTTTCCGAAG | A573413H | +G*+A*+T*G*G*T*T*T*T*C*C*G*A*A*G*T*+ | 85611 | 19 |
| | TTTT | | T*+T*+T | | |
| 3416 | TGATGGTTTTCCGAA | A573414H | +T*+G*+A*T*G*G*T*T*T*T*C*C*G*A*A*G*+ | 85612 | 19 |
| | GTTT | | T*+T*+T | | |
| 3417 | ATGATGGTTTTCCGA | A573415H | +A*+T*+G*A*T*G*G*T*T*T*T*C*C*G*A*A*+ | 85613 | 19 |
| | AGTT | | G*+T*+T | | |
| 3418 | ATGATGGTTTTCCGA | A573416H | +A*+T*+G*A*T*G*G*T*T*T*T*C*C*G*A*+A* | 85614 | 18 |
| | AGT | | +G*+T | | |
| 3419 | TATGATGGTTTTCCG | A573417H | +T*+A*+T*G*A*T*G*G*T*T*T*T*C*C*G*A*+ | 85614 | 19 |
| | AAGT | | A*+G*+T | | |
| 3420 | TGAAATTAAGGCTCT | A573418H | +T*+G*+A*A*A*T*T*A*A*G*G*C*T*C*T*+C* | 85634 | 18 |
| | CGT | | +G*+T | | |
| 3421 | TTGAAATTAAGGCTC | A573419H | +T*+T*+G*A*A*A*T*T*A*A*G*G*C*T*C*T*+ | 85634 | 19 |
| | TCGT | | C*+G*+T | | |
| 3422 | CTCTACCAGCCTACC | A573420Hi | +C*+T*+C*T*A*C*C*A*G*C*C*T*A*C*C*+A* | 85658 | 18 |
| | ATG | | +T*+G | | |
| 3423 | GAGTTGCTCACTTCT | A573421Hi | +G*+A*+G*T*T*G*C*T*C*A*C*T*T*C*T*C*+ | 85720 | 19 |
| | CAAA | | A*+A*+A | | |
| 3424 | GAGTTGCTCACTTCT | A573422Hi | +G*+A*+G*T*T*G*C*T*C*A*C*T*T*C*T*+C* | 85721 | 18 |
| | CAA | | +A*+A | | |
| 3425 | AGACCTCAGTATTCA | A573423Hi | +A*+G*+A*C*C*T*C*A*G*T*A*T*T*C*A*C*+ | 85992 | 19 |
| | CCTT | | C*+T*+T | | |
| 3426 | GTCTTCATGACTAAA | A573424Hi | +G*+T*+C*T*T*C*A*T*G*A*C*T*A*A*A*T*+ | 86179 | 19 |
| | TAAA | | A*+A*+A | | |
| 3427 | AGTGGTCTTCATGAC | A573425Hi | +A*+G*+T*G*G*T*C*T*T*C*A*T*G*A*C*T*+ | 86183 | 19 |
| | TAAA | | A*+A*+A | | |
| 3428 | AGTGGTCTTCATGAC | A573426Hi | +A*+G*+T*G*G*T*C*T*T*C*A*T*G*A*C*+T* | 86184 | 18 |
| | TAA | | +A*+A | | |
| 3429 | TGAACTAAGTAATTA | A573427Hi | +T*+G*+A*A*C*T*A*A*G*T*A*A*T*T*A*G*+ | 86223 | 19 |
| | GTGA | | T*+G*+A | | |
| 3430 | GAACTAAGTAATTAG | A573428Hi | +G*+A*+A*C*T*A*A*G*T*A*A*T*T*A*G*+T* | 86223 | 18 |
| | TGA | | +G*+A | | |
| 3431 | GTACAGTTTACACCA | A573429Hi | +G*+T*+A*C*A*G*T*T*T*A*C*A*C*C*A*C*+ | 86405 | 19 |
| | CACT | | A*+C*+T | | |
| 3432 | GTACAGTTTACACCA | A573430Hi | +G*+T*+A*C*A*G*T*T*T*A*C*A*C*C*A*+C* | 86406 | 18 |
| | CAC | | +A*+C | | |
| 3433 | CAGTACAGTTTACAC | A573431Hi | +C*+A*+G*T*A*C*A*G*T*T*T*A*C*A*C*C*+ | 86407 | 19 |
| | CACA | | A*+C*+A | | |
| 3434 | AGTACAGTTTACACC | A573432Hi | +A*+G*+T*A*C*A*G*T*T*T*A*C*A*C*C*+A* | 86407 | 18 |
| | ACA | | +C*+A | | |
| 3435 | TCAGTACAGTTTACA | A573433Hi | +T*+C*+A*G*T*A*C*A*G*T*T*T*A*C*A*C*+ | 86408 | 19 |
| | CCAC | | C*+A*+C | | |
| 3436 | TTTCAGTACAGTTTA | A573434Hi | +T*+T*+T*C*A*G*T*A*C*A*G*T*T*T*A*C*+ | 86410 | 19 |
| | CACC | | A*+C*+C | | |
| 3437 | GTCATATGGAACTAT | A573435Hi | +G*+T*+C*A*T*A*T*G*G*A*A*C*T*A*T*C*+ | 86435 | 19 |
| | CAAG | | A*+A*+G | | |
| 3438 | ACTAAGTGAAATTCA | A573436Hi | +A*+C*+T*A*A*G*T*G*A*A*A*T*T*C*A*T*+ | 86461 | 19 |
| | TCAT | | C*+A*+T | | |
| 3439 | AATAGGACTCTATTT | A573437Hi | +A*+A*+T*A*G*G*A*C*T*C*T*A*T*T*T*+A* | 86525 | 18 |
| | ATC | | +T*+C | | |
| 3440 | GGAGTTATAACCAAG | A573438Hi | +G*+G*+A*G*T*T*A*T*A*A*C*C*A*A*G*+G* | 86629 | 18 |
| | GTG | | +T*+G | | |
| 3441 | AGCACACATAATCTA | A573439Hi | +A*+G*+C*A*C*A*C*A*T*A*A*T*C*T*A*T*+ | 86679 | 19 |
| | TATA | | A*+T*+A | | |
| 3442 | TACAAATGGCTCATT | A573440Hi | +T*+A*+C*A*A*A*T*G*G*C*T*C*A*T*T*A*+ | 86708 | 19 |
| | AGGT | | G*+G*+T | | |
| 3443 | ATTACATGTCCAACA | A573441Hi | +A*+T*+T*A*C*A*T*G*T*C*C*A*A*C*A*+A* | 86878 | 18 |
| | AGA | | +G*+A | | |
| 3444 | AATTACATGTCCAAC | A573442Hi | +A*+A*+T*T*A*C*A*T*G*T*C*C*A*A*C*A*+ | 86878 | 19 |
| | AAGA | | A*+G*+A | | |
| 3445 | TAATTACATGTCCAA | A573443Hi | +T*+A*+A*T*T*A*C*A*T*G*T*C*C*A*A*C*+ | 86879 | 19 |
| | CAAG | | A*+A*+G | | |
| 3446 | ATCAACTAAATTCAG | A573444Hi | +A*+T*+C*A*A*C*T*A*A*A*T*T*C*A*G*G*+ | 87110 | 19 |
| | GCCT | | C*+C*+T | | |
| 3447 | ATCAACTAAATTCAG | A573445Hi | +A*+T*+C*A*A*C*T*A*A*A*T*T*C*A*G*+G* | 87111 | 18 |
| | GCC | | +C*+C | | |
| 3448 | CATCAACTAAATTCA | A573446Hi | +C*+A*+T*C*A*A*C*T*A*A*A*T*T*C*A*+G* | 87112 | 18 |
| | GGC | | +G*+C | | |
| 3449 | GCATCAACTAAATTC | A573447Hi | +G*+C*+A*T*C*A*A*C*T*A*A*A*T*T*C*+A* | 87113 | 18 |
| | AGG | | +G*+G | | |
| 3450 | TGCATCAACTAAATT | A573448Hi | +T*+G*+C*A*T*C*A*A*C*T*A*A*A*T*T*C*+ | 87113 | 19 |
| | CAGG | | A*+G*+G | | |
| 3451 | TGCATCAACTAAATT | A573449Hi | +T*+G*+C*A*T*C*A*A*C*T*A*A*A*T*T*+C* | 87114 | 18 |
| | CAG | | +A*+G | | |
| 3452 | TTTGCATCAACTAAA | A573450Hi | +T*+T*+T*G*C*A*T*C*A*A*C*T*A*A*A*T*+ | 87115 | 19 |
| | TTCA | | T*+C*+A | | |
| 3453 | TCTGTGACTTTGCAT | A573451Hi | +T*+C*+T*G*T*G*A*C*T*T*T*G*C*A*T*C*+ | 87123 | 19 |
| | CAAC | | A*+A*+C | | |
| 3454 | CAGTATTAATACAGC | A573452Hi | +C*+A*+G*T*A*T*T*A*A*T*A*C*A*G*C*T*+ | 87154 | 19 |
| | TTAA | | T*+A*+A | | |
| 3455 | GGTAATGATATCTAT | A573453Hi | +G*+G*+T*A*A*T*G*A*T*A*T*C*T*A*T*+T* | 87180 | 18 |
| | TAG | | +A*+G | | |
| 3456 | TTGGTAATGATATCT | A573454Hi | +T*+T*+G*G*T*A*A*T*G*A*T*A*T*C*T*A*+ | 87181 | 19 |
| | ATTA | | T*+T*+A | | |
| 3457 | ATTGGTAATGATATC | A573455Hi | +A*+T*+T*G*G*T*A*A*T*G*A*T*A*T*C*T*+ | 87182 | 19 |
| | TATT | | A*+T*+T | | |
| 3458 | ATTGGTAATGATATC | A573456Hi | +A*+T*+T*G*G*T*A*A*T*G*A*T*A*T*C*+T* | 87183 | 18 |
| | TAT | | +A*+T | | |
| 3459 | TGCATCCATTAGCAA | A573457Hi | +T*+G*+C*A*T*C*C*A*T*T*A*G*C*A*A*T*+ | 87312 | 19 |
| | TATA | | A*+T*+A | | |
| 3460 | CAGAATGCTGCATCC | A573458Hi | +C*+A*+G*A*A*T*G*C*T*G*C*A*T*C*C*A*+ | 87320 | 19 |
| | ATTA | | T*+T*+A | | |
| 3461 | ACCATTATGCCAGAA | A573459Hi | +A*+C*+C*A*T*T*A*T*G*C*C*A*G*A*A*T*+ | 87330 | 19 |
| | TGCT | | G*+C*+T | | |
| 3462 | AACCATTATGCCAGA | A573460Hi | +A*+A*+C*C*A*T*T*A*T*G*C*C*A*G*A*A*+ | 87331 | 19 |
| | ATGC | | T*+G*+C | | |
| 3463 | AAACCATTATGCCAG | A573461Hi | +A*+A*+A*C*C*A*T*T*A*T*G*C*C*A*G*A*+ | 87332 | 19 |
| | AATG | | A*+T*+G | | |
| 3464 | CAAACCATTATGCCA | A573462Hi | +C*+A*+A*A*C*C*A*T*T*A*T*G*C*C*A*G*+ | 87333 | 19 |
| | GAAT | | A*+A*+T | | |
| 3465 | TCTACAAAGCAATCA | A573463Hi | +T*+C*+T*A*C*A*A*A*G*C*A*A*T*C*A*A*+ | 87358 | 19 |
| | AATG | | A*+T*+G | | |
| 3466 | CAGGTTGAAGCACCT | A573464Hi | +C*+A*+G*G*T*T*G*A*A*G*C*A*C*C*T*C*+ | 87440 | 19 |
| | CAGG | | A*+G*+G | | |
| 3467 | ATTCAGGTTGAAGCA | A573465Hi | +A*+T*+T*C*A*G*G*T*T*G*A*A*G*C*A*C*+ | 87443 | 19 |
| | CCTC | | C*+T*+C | | |
| 3468 | CTGCTCTTTGAATTC | A573466Hi | +C*+T*+G*C*T*C*T*T*T*G*A*A*T*T*C*A*+ | 87454 | 19 |
| | AGGT | | G*+G*+T | | |
| 3469 | AGCTGCTCTTTGAAT | A573467Hi | +A*+G*+C*T*G*C*T*C*T*T*T*G*A*A*T*T*+ | 87456 | 19 |
| | TCAG | | C*+A*+G | | |
| 3470 | AGATTAGAGAAGAGC | A573468Hi | +A*+G*+A*T*T*A*G*A*G*A*A*G*A*G*C*T*+ | 87534 | 19 |
| | TAAG | | A*+A*+G | | |
| 3471 | TTAATAAGGTTGAGA | A573469Hi | +T*+T*+A*A*T*A*A*G*G*T*T*G*A*G*A*C*+ | 87556 | 19 |
| | CAGA | | A*+G*+A | | |
| 3472 | AAAGTGTCTGCACAG | A573470Hi | +A*+A*+A*G*T*G*T*C*T*G*C*A*C*A*G*A*+ | 87578 | 19 |
| | ACAC | | C*+A*+C | | |
| 3473 | ATGCCTGCTCTGCTA | A573471Hi | +A*+T*+G*C*C*T*G*C*T*C*T*G*C*T*A*A*+ | 87708 | 19 |
| | ATGG | | T*+G*+G | | |
| 3474 | TTACTCAAGAGACTG | A573472Hi | +T*+T*+A*C*T*C*A*A*G*A*G*A*C*T*G*+T* | 87957 | 18 |
| | TTT | | +T*+T | | |
| 3475 | AATGGTTACTCAAGA | A573473Hi | +A*+A*+T*G*G*T*T*A*C*T*C*A*A*G*A*G*+ | 87961 | 19 |
| | GACT | | A*+C*+T | | |
| 3476 | GACAACCATAACCAT | A573474Hi | +G*+A*+C*A*A*C*C*A*T*A*A*C*C*A*T*+T* | 87997 | 18 |
| | TAC | | +A*+C | | |
| 3477 | AGACAACCATAACCA | A573475Hi | +A*+G*+A*C*A*A*C*C*A*T*A*A*C*C*A*+T* | 87998 | 18 |
| | TTA | | +T*+A | | |
| 3478 | TATGGAAATTACATA | A573476Hi | +T*+A*+T*G*G*A*A*A*T*T*A*C*A*T*A*T*+ | 88139 | 19 |
| | TTCT | | T*+C*+T | | |
| 3479 | ACCAGATGGCAAACT | A573477Hi | +A*+C*+C*A*G*A*T*G*G*C*A*A*A*C*T*C*+ | 88171 | 19 |
| | CTCC | | T*+C*+C | | |
| 3480 | CACCAGATGGCAAAC | A573478Hi | +C*+A*+C*C*A*G*A*T*G*G*C*A*A*A*C*T*+ | 88172 | 19 |
| | TCTC | | C*+T*+C | | |
| 3481 | GGATGATCATTGTTC | A573479Hi | +G*+G*+A*T*G*A*T*C*A*T*T*G*T*T*C*+T* | 88366 | 18 |
| | TGT | | +G*+T | | |
| 3482 | GAGGATGATCATTGT | A573480Hi | +G*+A*+G*G*A*T*G*A*T*C*A*T*T*G*T*T*+ | 88367 | 19 |
| | TCTG | | C*+T*+G | | |
| 3483 | GGAGAGGATGATCAT | A573481Hi | +G*+G*+A*G*A*G*G*A*T*G*A*T*C*A*T*T*+ | 88370 | 19 |
| | TGTT | | G*+T*+T | | |
| 3484 | GAGAGGATGATCATT | A573482Hi | +G*+A*+G*A*G*G*A*T*G*A*T*C*A*T*T*+G* | 88370 | 18 |
| | GTT | | +T*+T | | |
| 3485 | AGGAGAGGATGATCA | A573483Hi | +A*+G*+G*A*G*A*G*G*A*T*G*A*T*C*A*T*+ | 88371 | 19 |
| | TTGT | | T*+G*+T | | |
| 3486 | AGGAGAGGATGATCA | A573484Hi | +A*+G*+G*A*G*A*G*G*A*T*G*A*T*C*A*+T* | 88372 | 18 |
| | TTG | | +T*+G | | |
| 3487 | TAGGAGAGGATGATC | A573485Hi | +T*+A*+G*G*A*G*A*G*G*A*T*G*A*T*C*A*+ | 88372 | 19 |
| | ATTG | | T*+T*+G | | |
| 3488 | TACAAATTACAAGAG | A573486Hi | +T*+A*+C*A*A*A*T*T*A*C*A*A*G*A*G*C*+ | 88584 | 19 |
| | CTTT | | T*+T*+T | | |
| 3489 | GTACAAATTACAAGA | A573487Hi | +G*+T*+A*C*A*A*A*T*T*A*C*A*A*G*A*G*+ | 88585 | 19 |
| | GCTT | | C*+T*+T | | |
| 3490 | AGTACAAATTACAAG | A573488Hi | +A*+G*+T*A*C*A*A*A*T*T*A*C*A*A*G*A*+ | 88586 | 19 |
| | AGCT | | G*+C*+T | | |
| 3491 | CAGTACAAATTACAA | A573489Hi | +C*+A*+G*T*A*C*A*A*A*T*T*A*C*A*A*G*+ | 88587 | 19 |
| | GAGC | | A*+G*+C | | |
| 3492 | GATATCAGTACAAAT | A573490Hi | +G*+A*+T*A*T*C*A*G*T*A*C*A*A*A*T*+T* | 88593 | 18 |
| | TAC | | +A*+C | | |
| 3493 | ATGTGAAACAGACAC | A573491Hi | +A*+T*+G*T*G*A*A*A*C*A*G*A*C*A*C*G*+ | 88617 | 19 |
| | GCTA | | C*+T*+A | | |
| 3494 | TGTGAAACAGACACG | A573492Hi | +T*+G*+T*G*A*A*A*C*A*G*A*C*A*C*G*+C* | 88617 | 18 |
| | CTA | | +T*+A | | |
| 3495 | TAATGTGAAACAGAC | A573493Hi | +T*+A*+A*T*G*T*G*A*A*A*C*A*G*A*C*A*+ | 88619 | 19 |
| | ACGC | | C*+G*+C | | |
| 3496 | ACTTAATGTGAAACA | A573494Hi | +A*+C*+T*T*A*A*T*G*T*G*A*A*A*C*A*G*+ | 88622 | 19 |
| | GACA | | A*+C*+A | | |
| 3497 | TAAACTTAATGTGAA | A573495Hi | +T*+A*+A*A*C*T*T*A*A*T*G*T*G*A*A*A*+ | 88625 | 19 |
| | ACAG | | C*+A*+G | | |
| 3498 | ATTAAACTTAATGTG | A573496Hi | +A*+T*+T*A*A*A*C*T*T*A*A*T*G*T*G*A*+ | 88627 | 19 |
| | AAAC | | A*+A*+C | | |
| 3499 | CATTAAACTTAATGT | A573497Hi | +C*+A*+T*T*A*A*A*C*T*T*A*A*T*G*T*G*+ | 88628 | 19 |
| | GAAA | | A*+A*+A | | |
| 3500 | CAAATTCAGTGCTAA | A573498Hi | +C*+A*+A*A*T*T*C*A*G*T*G*C*T*A*A*C*+ | 88893 | 19 |
| | CAGT | | A*+G*+T | | |
| 3501 | TGGCAAATTCAGTGC | A573499Hi | +T*+G*+G*C*A*A*A*T*T*C*A*G*T*G*C*+T* | 88897 | 18 |
| | TAA | | +A*+A | | |
| 3502 | TTGGCAAATTCAGTG | A573500Hi | +T*+T*+G*G*C*A*A*A*T*T*C*A*G*T*G*C*+ | 88897 | 19 |
| | CTAA | | T*+A*+A | | |
| 3503 | GTTGGCAAATTCAGT | A573501Hi | +G*+T*+T*G*G*C*A*A*A*T*T*C*A*G*T*G*+ | 88898 | 19 |
| | GCTA | | C*+T*+A | | |
| 3504 | TGGTTGGCAAATTCA | A573502Hi | +T*+G*+G*T*T*G*G*C*A*A*A*T*T*C*A*G*+ | 88900 | 19 |
| | GTGC | | T*+G*+C | | |
| 3505 | GGTTGGCAAATTCAG | A573503Hi | +G*+G*+T*T*G*G*C*A*A*A*T*T*C*A*G*+T* | 88900 | 18 |
| | TGC | | +G*+C | | |
| 3506 | ATGGTTGGCAAATTC | A573504Hi | +A*+T*+G*G*T*T*G*G*C*A*A*A*T*T*C*+A* | 88902 | 18 |
| | AGT | | +G*+T | | |
| 3507 | CAAATGACAAGCCGT | A573505Hi | +C*+A*+A*A*T*G*A*C*A*A*G*C*C*G*T*+A* | 88947 | 18 |
| | AAA | | +A*+A | | |
| 3508 | ACAAATGACAAGCCG | A573506Hi | +A*+C*+A*A*A*T*G*A*C*A*A*G*C*C*G*T*+ | 88947 | 19 |
| | TAAA | | A*+A*+A | | |
| 3509 | ACAAATGACAAGCCG | A573507Hi | +A*+C*+A*A*A*T*G*A*C*A*A*G*C*C*G*+T* | 88948 | 18 |
| | TAA | | +A*+A | | |
| 3510 | TACAAATGACAAGCC | A573508Hi | +T*+A*+C*A*A*A*T*G*A*C*A*A*G*C*C*G*+ | 88948 | 19 |
| | GTAA | | T*+A*+A | | |
| 3511 | TTACAAATGACAAGC | A573509Hi | +T*+T*+A*C*A*A*A*T*G*A*C*A*A*G*C*C*+ | 88949 | 19 |
| | CGTA | | G*+T*+A | | |
| 3512 | ATTACAAATGACAAG | A573510Hi | +A*+T*+T*A*C*A*A*A*T*G*A*C*A*A*G*C*+ | 88950 | 19 |
| | CCGT | | C*+G*+T | | |
| 3513 | AATTACAAATGACAA | A573511Hi | +A*+A*+T*T*A*C*A*A*A*T*G*A*C*A*A*G*+ | 88951 | 19 |
| | GCCG | | C*+C*+G | | |
| 3514 | ATTACAAATGACAAG | A573512Hi | +A*+T*+T*A*C*A*A*A*T*G*A*C*A*A*G*+C* | 88951 | 18 |
| | CCG | | +C*+G | | |
| 3515 | AAATTACAAATGACA | A573513Hi | +A*+A*+A*T*T*A*C*A*A*A*T*G*A*C*A*A*+ | 88952 | 19 |
| | AGCC | | G*+C*+C | | |
| 3516 | GAATCAGCTGTCCAA | A573514H | +G*+A*+A*T*C*A*G*C*T*G*T*C*C*A*A*C*+ | 88991 | 19 |
| | CAAC | | A*+A*+C | | |
| 3517 | GGAATCAGCTGTCCA | A573515H | +G*+G*+A*A*T*C*A*G*C*T*G*T*C*C*A*A*+ | 88992 | 19 |
| | ACAA | | C*+A*+A | | |
| 3518 | GGAATCAGCTGTCCA | A573516H | +G*+G*+A*A*T*C*A*G*C*T*G*T*C*C*A*+A* | 88993 | 18 |
| | ACA | | +C*+A | | |
| 3519 | TGGAATCAGCTGTCC | A573517H | +T*+G*+G*A*A*T*C*A*G*C*T*G*T*C*C*A*+ | 88993 | 19 |
| | AACA | | A*+C*+A | | |
| 3520 | AGTCTGGAATCAGCT | A573518H | +A*+G*+T*C*T*G*G*A*A*T*C*A*G*C*T*G*+ | 88997 | 19 |
| | GTCC | | T*+C*+C | | |
| 3521 | CAGTCTGGAATCAGC | A573519H | +C*+A*+G*T*C*T*G*G*A*A*T*C*A*G*C*T*+ | 88998 | 19 |
| | TGTC | | G*+T*+C | | |
| 3522 | CAGTCTGGAATCAGC | A573520H | +C*+A*+G*T*C*T*G*G*A*A*T*C*A*G*C*+T* | 88999 | 18 |
| | TGT | | +G*+T | | |
| 3523 | GCAGTCTGGAATCAG | A573521H | +G*+C*+A*G*T*C*T*G*G*A*A*T*C*A*G*C*+ | 88999 | 19 |
| | CTGT | | T*+G*+T | | |
| 3524 | GCAGTCTGGAATCAG | A573522H | +G*+C*+A*G*T*C*T*G*G*A*A*T*C*A*G*+C* | 89000 | 18 |
| | CTG | | +T*+G | | |
| 3525 | AGCAGTCTGGAATCA | A573523H | +A*+G*+C*A*G*T*C*T*G*G*A*A*T*C*A*G*+ | 89000 | 19 |
| | GCTG | | C*+T*+G | | |
| 3526 | TAGCAGTCTGGAATC | A573524H | +T*+A*+G*C*A*G*T*C*T*G*G*A*A*T*C*A*+ | 89001 | 19 |
| | AGCT | | G*+C*+T | | |
| 3527 | CATAGCAGTCTGGAA | A573525H | +C*+A*+T*A*G*C*A*G*T*C*T*G*G*A*A*T*+ | 89003 | 19 |
| | TCAG | | C*+A*+G | | |
| 3528 | ACATAGCAGTCTGGA | A573526H | +A*+C*+A*T*A*G*C*A*G*T*C*T*G*G*A*A*+ | 89004 | 19 |
| | ATCA | | T*+C*+A | | |
| 3529 | GTTCTACATAGCAGT | A573527H | +G*+T*+T*C*T*A*C*A*T*A*G*C*A*G*T*C*+ | 89009 | 19 |
| | CTGG | | T*+G*+G | | |
| 3530 | AAGTTCTACATAGCA | A573528H | +A*+A*+G*T*T*C*T*A*C*A*T*A*G*C*A*G*+ | 89011 | 19 |
| | GTCT | | T*+C*+T | | |
| 3531 | CAAGTTCTACATAGC | A573529H | +C*+A*+A*G*T*T*C*T*A*C*A*T*A*G*C*A*+ | 89012 | 19 |
| | AGTC | | G*+T*+C | | |
| 3532 | TCAAGTTCTACATAG | A573530H | +T*+C*+A*A*G*T*T*C*T*A*C*A*T*A*G*C*+ | 89013 | 19 |
| | CAGT | | A*+G*+T | | |
| 3533 | CGCTCCGATAAAATG | A573531H | +C*+G*+C*T*C*C*G*A*T*A*A*A*A*T*G*A*+ | 89034 | 19 |
| | ATTT | | T*+T*+T | | |
| 3534 | CGCTCCGATAAAATG | A573532H | +C*+G*+C*T*C*C*G*A*T*A*A*A*A*T*G*+A* | 89035 | 18 |
| | ATT | | +T*+T | | |
| 3535 | ACGCTCCGATAAAAT | A573533H | +A*+C*+G*C*T*C*C*G*A*T*A*A*A*A*T*G*+ | 89035 | 19 |
| | GATT | | A*+T*+T | | |
| 3536 | CGCTCCGATAAAATG | A573534H | +C*+G*+C*T*C*C*G*A*T*A*A*A*A*T*+G*+A | 89036 | 17 |
| | AT | | *+T | | |
| 3537 | ACGCTCCGATAAAAT | A573535H | +A*+C*+G*C*T*C*C*G*A*T*A*A*A*A*T*+G* | 89036 | 18 |
| | GAT | | +A*+T | | |
| 3538 | TACGCTCCGATAAAA | A573536H | +T*+A*+C*G*C*T*C*C*G*A*T*A*A*A*A*T*+ | 89036 | 19 |
| | TGAT | | G*+A*+T | | |
| 3539 | ACGCTCCGATAAAAT | A573537H | +A*+C*+G*C*T*C*C*G*A*T*A*A*A*A*+T*+G | 89037 | 17 |
| | GA | | *+A | | |
| 3540 | TACGCTCCGATAAAA | A573538H | +T*+A*+C*G*C*T*C*C*G*A*T*A*A*A*A*+T* | 89037 | 18 |
| | TGA | | +G*+A | | |
| 3541 | TTACGCTCCGATAAA | A573539H | +T*+T*+A*C*G*C*T*C*C*G*A*T*A*A*A*A*+ | 89037 | 19 |
| | ATGA | | T*+G*+A | | |
| 3542 | TACGCTCCGATAAAA | A573540H | +T*+A*+C*G*C*T*C*C*G*A*T*A*A*A*+A*+T | 89038 | 17 |
| | TG | | *+G | | |
| 3543 | TTACGCTCCGATAAA | A573541H | +T*+T*+A*C*G*C*T*C*C*G*A*T*A*A*A*+A* | 89038 | 18 |
| | ATG | | +T*+G | | |
| 3544 | TTTACGCTCCGATAA | A573542H | +T*+T*+T*A*C*G*C*T*C*C*G*A*T*A*A*A*+ | 89038 | 19 |
| | AATG | | A*+T*+G | | |
| 3545 | TTTTTACGCTCCGAT | A573543H | +T*+T*+T*T*T*A*C*G*C*T*C*C*G*A*T*A*+ | 89040 | 19 |
| | AAAA | | A*+A*+A | | |
| 3546 | TTTTTACGCTCCGAT | A573544H | +T*+T*+T*T*T*A*C*G*C*T*C*C*G*A*T*+A* | 89041 | 18 |
| | AAA | | +A*+A | | |
| 3547 | ATTTTTACGCTCCGA | A573545H | +A*+T*+T*T*T*T*A*C*G*C*T*C*C*G*A*T*+ | 89041 | 19 |
| | TAAA | | A*+A*+A | | |
| 3548 | TTTTTACGCTCCGAT | A573546H | +T*+T*+T*T*T*A*C*G*C*T*C*C*G*A*+T*+A | 89042 | 17 |
| | AA | | *+A | | |
| 3549 | ATTTTTACGCTCCGA | A573547H | +A*+T*+T*T*T*T*A*C*G*C*T*C*C*G*A*+T* | 89042 | 18 |
| | TAA | | +A*+A | | |
| 3550 | CATTTTTACGCTCCG | A573548H | +C*+A*+T*T*T*T*T*A*C*G*C*T*C*C*G*A*+ | 89042 | 19 |
| | ATAA | | T*+A*+A | | |
| 3551 | TTTTTACGCTCCGAT | A573549H | +T*+T*+T*T*T*A*C*G*C*T*C*C*G*+A*+T*+ | 89043 | 16 |
| | A | | A | | |
| 3552 | ATTTTTACGCTCCGA | A573550H | +A*+T*+T*T*T*T*A*C*G*C*T*C*C*G*+A*+T | 89043 | 17 |
| | TA | | *+A | | |
| 3553 | CATTTTTACGCTCCG | A573551H | +C*+A*+T*T*T*T*T*A*C*G*C*T*C*C*G*+A* | 89043 | 18 |
| | ATA | | +T*+A | | |
| 3554 | ACATTTTTACGCTCC | A573552H | +A*+C*+A*T*T*T*T*T*A*C*G*C*T*C*C*G*+ | 89043 | 19 |
| | GATA | | A*+T*+A | | |
| 3555 | ATTTTTACGCTCCGA | A573553H | +A*+T*+T*T*T*T*A*C*G*C*T*C*C*+G*+A*+ | 89044 | 16 |
| | T | | T | | |
| 3556 | CATTTTTACGCTCCG | A573554H | +C*+A*+T*T*T*T*T*A*C*G*C*T*C*C*+G*+A | 89044 | 17 |
| | AT | | *+T | | |
| 3557 | ACATTTTTACGCTCC | A573555H | +A*+C*+A*T*T*T*T*T*A*C*G*C*T*C*C*+G* | 89044 | 18 |
| | GAT | | +A*+T | | |
| 3558 | CACATTTTTACGCTC | A573556H | +C*+A*+C*A*T*T*T*T*T*A*C*G*C*T*C*C*+ | 89044 | 19 |
| | CGAT | | G*+A*+T | | |
| 3559 | ACATTTTTACGCTCC | A573557H | +A*+C*+A*T*T*T*T*T*A*C*G*C*T*C*+C*+G | 89045 | 17 |
| | GA | | *+A | | |
| 3560 | CACATTTTTACGCTC | A573558H | +C*+A*+C*A*T*T*T*T*T*A*C*G*C*T*C*+C* | 89045 | 18 |
| | CGA | | +G*+A | | |
| 3561 | GCACATTTTTACGCT | A573559H | +G*+C*+A*C*A*T*T*T*T*T*A*C*G*C*T*C*+ | 89045 | 19 |
| | CCGA | | C*+G*+A | | |
| 3562 | GCACATTTTTACGCT | A573560H | +G*+C*+A*C*A*T*T*T*T*T*A*C*G*C*T*+C* | 89046 | 18 |
| | CCG | | +C*+G | | |
| 3563 | GGCACATTTTTACGC | A573561H | +G*+G*+C*A*C*A*T*T*T*T*T*A*C*G*C*T*+ | 89046 | 19 |
| | TCCG | | C*+C*+G | | |
| 3564 | GCACATTTTTACGCT | A573562H | +G*+C*+A*C*A*T*T*T*T*T*A*C*G*C*+T*+C | 89047 | 17 |
| | CC | | *+C | | |
| 3565 | GGCACATTTTTACGC | A573563H | +G*+G*+C*A*C*A*T*T*T*T*T*A*C*G*C*+T* | 89047 | 18 |
| | TCC | | +C*+C | | |
| 3566 | TGGCACATTTTTACG | A573564H | +T*+G*+G*C*A*C*A*T*T*T*T*T*A*C*G*C*+ | 89047 | 19 |
| | CTCC | | T*+C*+C | | |
| 3567 | TTGGCACATTTTTAC | A573565H | +T*+T*+G*G*C*A*C*A*T*T*T*T*T*A*C*G*+ | 89048 | 19 |
| | GCTC | | C*+T*+C | | |
| 3568 | TTGGCACATTTTTAC | A573566H | +T*+T*+G*G*C*A*C*A*T*T*T*T*T*A*C*+G* | 89049 | 18 |
| | GCT | | +C*+T | | |
| 3569 | ATTGGCACATTTTTA | A573567H | +A*+T*+T*G*G*C*A*C*A*T*T*T*T*T*A*C*+ | 89049 | 19 |
| | CGCT | | G*+C*+T | | |
| 3570 | TTGGCACATTTTTAC | A573568H | +T*+T*+G*G*C*A*C*A*T*T*T*T*T*A*+C*+G | 89050 | 17 |
| | GC | | *+C | | |
| 3571 | ATTGGCACATTTTTA | A573569H | +A*+T*+T*G*G*C*A*C*A*T*T*T*T*T*A*+C* | 89050 | 18 |
| | CGC | | +G*+C | | |
| 3572 | AATTGGCACATTTTT | A573570H | +A*+A*+T*T*G*G*C*A*C*A*T*T*T*T*T*A*+ | 89050 | 19 |
| | ACGC | | C*+G*+C | | |
| 3573 | CAATTGGCACATTTT | A573571H | +C*+A*+A*T*T*G*G*C*A*C*A*T*T*T*T*T*+ | 89051 | 19 |
| | TACG | | A*+C*+G | | |
| 3574 | GGAAATTCAATTGGC | A573572H | +G*+G*+A*A*A*T*T*C*A*A*T*T*G*G*C*A*+ | 89058 | 19 |
| | ACAT | | C*+A*+T | | |
| 3575 | TAGTTGTAATAATCG | A573573H | +T*+A*+G*T*T*G*T*A*A*T*A*A*T*C*G*T*+ | 89089 | 19 |
| | TTTC | | T*+T*+C | | |
| 3576 | CTAGTTGTAATAATC | A573574H | +C*+T*+A*G*T*T*G*T*A*A*T*A*A*T*C*G*+ | 89090 | 19 |
| | GTTT | | T*+T*+T | | |
| 3577 | ACTAGTTGTAATAAT | A573575H | +A*+C*+T*A*G*T*T*G*T*A*A*T*A*A*T*C*+ | 89091 | 19 |
| | CGTT | | G*+T*+T | | |
| 3578 | ACTAGTTGTAATAAT | A573576H | +A*+C*+T*A*G*T*T*G*T*A*A*T*A*A*T*+C* | 89092 | 18 |
| | CGT | | +G*+T | | |
| 3579 | CACTAGTTGTAATAA | A573577H | +C*+A*+C*T*A*G*T*T*G*T*A*A*T*A*A*T*+ | 89092 | 19 |
| | TCGT | | C*+G*+T | | |
| 3580 | CACTAGTTGTAATAA | A573578H | +C*+A*+C*T*A*G*T*T*G*T*A*A*T*A*A*+T* | 89093 | 18 |
| | TCG | | +C*+G | | |
| 3581 | TCACTAGTTGTAATA | A573579H | +T*+C*+A*C*T*A*G*T*T*G*T*A*A*T*A*A*+ | 89093 | 19 |
| | ATCG | | T*+C*+G | | |
| 3582 | CTCACTAGTTGTAAT | A573580H | +C*+T*+C*A*C*T*A*G*T*T*G*T*A*A*T*A*+ | 89094 | 19 |
| | AATC | | A*+T*+C | | |
| 3583 | TCTCTCACTAGTTGT | A573581H | +T*+C*+T*C*T*C*A*C*T*A*G*T*T*G*T*A*+ | 89097 | 19 |
| | AATA | | A*+T*+A | | |
| 3584 | TTCTCTCACTAGTTGT | A573582H | +T*+T*+C*T*C*T*C*A*C*T*A*G*T*T*G*T*+ | 89098 | 19 |
| | AAT | | A*+A*+T | | |
| 3585 | TTTTCTCTCACTAGTT | A573583H | +T*+T*+T*T*C*T*C*T*C*A*C*T*A*G*T*T*+ | 89100 | 19 |
| | GTA | | G*+T*+A | | |
| 3586 | CTCATTTTCATCTAAC | A573584H | +C*+T*+C*A*T*T*T*T*C*A*T*C*T*A*A*C*+ | 89125 | 19 |
| | TGC | | T*+G*+C | | |
| 3587 | GCTCATTTTCATCTAA | A573585H | +G*+C*+T*C*A*T*T*T*T*C*A*T*C*T*A*A*+ | 89126 | 19 |
| | CTG | | C*+T*+G | | |
| 3588 | GAGGAAGCTCATTTT | A573586H | +G*+A*+G*G*A*A*G*C*T*C*A*T*T*T*T*C*+ | 89132 | 19 |
| | CATC | | A*+T*+C | | |
| 3589 | GTGAGGAAGCTCATT | A573587H | +G*+T*+G*A*G*G*A*A*G*C*T*C*A*T*T*T*+ | 89134 | 19 |
| | TTCA | | T*+C*+A | | |
| 3590 | CGTGAGGAAGCTCAT | A573588H | +C*+G*+T*G*A*G*G*A*A*G*C*T*C*A*T*T*+ | 89135 | 19 |
| | TTTC | | T*+T*+C | | |
| 3591 | GCGTGAGGAAGCTCA | A573589H | +G*+C*+G*T*G*A*G*G*A*A*G*C*T*C*A*T*+ | 89136 | 19 |
| | TTTT | | T*+T*+T | | |
| 3592 | TGCGTGAGGAAGCTC | A573590H | +T*+G*+C*G*T*G*A*G*G*A*A*G*C*T*C*A*+ | 89137 | 19 |
| | ATTT | | T*+T*+T | | |
| 3593 | TGCGTGAGGAAGCTC | A573591H | +T*+G*+C*G*T*G*A*G*G*A*A*G*C*T*C*+A* | 89138 | 18 |
| | ATT | | +T*+T | | |
| 3594 | CTGCGTGAGGAAGCT | A573592H | +C*+T*+G*C*G*T*G*A*G*G*A*A*G*C*T*C*+ | 89138 | 19 |
| | CATT | | A*+T*+T | | |
| 3595 | ACTGCGTGAGGAAGC | A573593H | +A*+C*+T*G*C*G*T*G*A*G*G*A*A*G*C*T*+ | 89139 | 19 |
| | TCAT | | C*+A*+T | | |
| 3596 | AACTGCGTGAGGAAG | A573594H | +A*+A*+C*T*G*C*G*T*G*A*G*G*A*A*G*C* | 89140 | 19 |
| | CTCA | | +T*+C*+A | | |
| 3597 | AACTGCGTGAGGAAG | A573595H | +A*+A*+C*T*G*C*G*T*G*A*G*G*A*A*G*+C | 89141 | 18 |
| | CTC | | *+T*+C | | |
| 3598 | GAACTGCGTGAGGAA | A573596H | +G*+A*+A*C*T*G*C*G*T*G*A*G*G*A*A*G* | 89141 | 19 |
| | GCTC | | +C*+T*+C | | |
| 3599 | TGAACTGCGTGAGGA | A573597H | +T*+G*+A*A*C*T*G*C*G*T*G*A*G*G*A*A*+ | 89142 | 19 |
| | AGCT | | G*+C*+T | | |
| 3600 | GTGAACTGCGTGAGG | A573598H | +G*+T*+G*A*A*C*T*G*C*G*T*G*A*G*G*A* | 89143 | 19 |
| | AAGC | | +A*+G*+C | | |
| 3601 | AAGTGAACTGCGTGA | A573599H | +A*+A*+G*T*G*A*A*C*T*G*C*G*T*G*A*G*+ | 89145 | 19 |
| | GGAA | | G*+A*+A | | |
| 3602 | TTAGAAAGTGAACTG | A573600H | +T*+T*+A*G*A*A*A*G*T*G*A*A*C*T*G*C*+ | 89150 | 19 |
| | CGTG | | G*+T*+G | | |
| 3603 | ATTTAGAAAGTGAAC | A573601H | +A*+T*+T*T*A*G*A*A*A*G*T*G*A*A*C*T*+ | 89152 | 19 |
| | TGCG | | G*+C*+G | | |
| 3604 | CATTTAGAAAGTGAA | A573602H | +C*+A*+T*T*T*A*G*A*A*A*G*T*G*A*A*C*+ | 89153 | 19 |
| | CTGC | | T*+G*+C | | |
| 3605 | TTACTCCTAGGTGAT | A573603Hi | +T*+T*+A*C*T*C*C*T*A*G*G*T*G*A*T*T*+ | 89378 | 19 |
| | TTCT | | T*+C*+T | | |
| 3606 | TTTACTCCTAGGTGA | A573604Hi | +T*+T*+T*A*C*T*C*C*T*A*G*G*T*G*A*T*+ | 89379 | 19 |
| | TTTC | | T*+T*+C | | |
| 3607 | GTTCCACAGCTCCTG | A573605Hi | +G*+T*+T*C*C*A*C*A*G*C*T*C*C*T*G*A*+ | 89458 | 19 |
| | AATA | | A*+T*+A | | |
| 3608 | AGCTAGATCTTCCAC | A573606Hi | +A*+G*+C*T*A*G*A*T*C*T*T*C*C*A*C*+T* | 89621 | 18 |
| | TCA | | +C*+A | | |
| 3609 | GAGCTAGATCTTCCA | A573607Hi | +G*+A*+G*C*T*A*G*A*T*C*T*T*C*C*A*+C* | 89622 | 18 |
| | CTC | | +T*+C | | |
| 3610 | AGAGCTAGATCTTCC | A573608Hi | +A*+G*+A*G*C*T*A*G*A*T*C*T*T*C*C*+A* | 89623 | 18 |
| | ACT | | +C*+T | | |
| 3611 | GAGAGCTAGATCTTC | A573609Hi | +G*+A*+G*A*G*C*T*A*G*A*T*C*T*T*C*C*+ | 89623 | 19 |
| | CACT | | A*+C*+T | | |
| 3612 | GAGAGCTAGATCTTC | A573610Hi | +G*+A*+G*A*G*C*T*A*G*A*T*C*T*T*C*+C* | 89624 | 18 |
| | CAC | | +A*+C | | |
| 3613 | ACAATTAGCACTGAA | A573611Hi | +A*+C*+A*A*T*T*A*G*C*A*C*T*G*A*A*G*+ | 89682 | 19 |
| | GTTG | | T*+T*+G | | |
| 3614 | CAATTAGCACTGAAG | A573612Hi | +C*+A*+A*T*T*A*G*C*A*C*T*G*A*A*G*+T* | 89682 | 18 |
| | TTG | | +T*+G | | |
| 3615 | TCAAGTACACAATTA | A573613Hi | +T*+C*+A*A*G*T*A*C*A*C*A*A*T*T*A*G*+ | 89690 | 19 |
| | GCAC | | C*+A*+C | | |
| 3616 | ATCAAGTACACAATT | A573614Hi | +A*+T*+C*A*A*G*T*A*C*A*C*A*A*T*T*A*+ | 89691 | 19 |
| | AGCA | | G*+C*+A | | |
| 3617 | GATCAAGTACACAAT | A573615Hi | +G*+A*+T*C*A*A*G*T*A*C*A*C*A*A*T*T*+ | 89692 | 19 |
| | TAGC | | A*+G*+C | | |
| 3618 | ATCAAGTACACAATT | A573616Hi | +A*+T*+C*A*A*G*T*A*C*A*C*A*A*T*T*+A* | 89692 | 18 |
| | AGC | | +G*+C | | |
| 3619 | ATGATCAAGTACACA | A573617Hi | +A*+T*+G*A*T*C*A*A*G*T*A*C*A*C*A*A*+ | 89694 | 19 |
| | ATTA | | T*+T*+A | | |
| 3620 | GTAATATGATCAAGT | A573618Hi | +G*+T*+A*A*T*A*T*G*A*T*C*A*A*G*T*A*+ | 89699 | 19 |
| | ACAC | | C*+A*+C | | |
| 3621 | CTATCAAACAACTCA | A573619Hi | +C*+T*+A*T*C*A*A*A*C*A*A*C*T*C*A*C*+ | 89729 | 19 |
| | CACA | | A*+C*+A | | |
| 3622 | TACTATCAAACAACT | A573620Hi | +T*+A*+C*T*A*T*C*A*A*A*C*A*A*C*T*C*+ | 89731 | 19 |
| | CACA | | A*+C*+A | | |
| 3623 | CTAGTACTATCAAAC | A573621Hi | +C*+T*+A*G*T*A*C*T*A*T*C*A*A*A*C*+A* | 89736 | 18 |
| | AAC | | +A*+C | | |
| 3624 | TACTCTAGTACTATC | A573622Hi | +T*+A*+C*T*C*T*A*G*T*A*C*T*A*T*C*A*+ | 89739 | 19 |
| | AAAC | | A*+A*+C | | |
| 3625 | TTACTCTAGTACTATC | A573623Hi | +T*+T*+A*C*T*C*T*A*G*T*A*C*T*A*T*C*+ | 89740 | 19 |
| | AAA | | A*+A*+A | | |
| 3626 | CCAGTCACTTACTCT | A573624Hi | +C*+C*+A*G*T*C*A*C*T*T*A*C*T*C*T*A*+ | 89748 | 19 |
| | AGTA | | G*+T*+A | | |
| 3627 | AGGAACAGATCATAA | A573625Hi | +A*+G*+G*A*A*C*A*G*A*T*C*A*T*A*A*A*+ | 89816 | 19 |
| | ACCA | | C*+C*+A | | |
| 3628 | CAGATAAGAAACACA | A573626Hi | +C*+A*+G*A*T*A*A*G*A*A*A*C*A*C*A*G*+ | 89866 | 19 |
| | GCAT | | C*+A*+T | | |
| 3629 | CAATATTATAATGGA | A573627Hi | +C*+A*+A*T*A*T*T*A*T*A*A*T*G*G*A*G*+ | 90032 | 19 |
| | GTAT | | T*+A*+T | | |
| 3630 | CCAATATTATAATGG | A573628Hi | +C*+C*+A*A*T*A*T*T*A*T*A*A*T*G*G*A*+ | 90033 | 19 |
| | AGTA | | G*+T*+A | | |
| 3631 | CCAATATTATAATGG | A573629Hi | +C*+C*+A*A*T*A*T*T*A*T*A*A*T*G*G*+A* | 90034 | 18 |
| | AGT | | +G*+T | | |
| 3632 | AGCTGTGCATCCTAT | A573630Hi | +A*+G*+C*T*G*T*G*C*A*T*C*C*T*A*T*G*+ | 90059 | 19 |
| | GGTC | | G*+T*+C | | |
| 3633 | GCTGTGCATCCTATG | A573631Hi | +G*+C*+T*G*T*G*C*A*T*C*C*T*A*T*G*+G* | 90059 | 18 |
| | GTC | | +T*+C | | |
| 3634 | TAGAAGCTGTGCATC | A573632Hi | +T*+A*+G*A*A*G*C*T*G*T*G*C*A*T*C*+C* | 90064 | 18 |
| | CTA | | +T*+A | | |
| 3635 | GACTAGAAGCTGTGC | A573633Hi | +G*+A*+C*T*A*G*A*A*G*C*T*G*T*G*C*+A* | 90067 | 18 |
| | ATC | | +T*+C | | |
| 3636 | ATCAAACAGTGAAAC | A573634Hi | +A*+T*+C*A*A*A*C*A*G*T*G*A*A*A*C*A*+ | 90182 | 19 |
| | ATGG | | T*+G*+G | | |
| 3637 | TGGGTCTTGAAAATG | A573635H | +T*+G*+G*G*T*C*T*T*G*A*A*A*A*T*G*A*+ | 90223 | 19 |
| | AAGA | | A*+G*+A | | |
| 3638 | GTGCTGGGTCTTGAA | A573636H | +G*+T*+G*C*T*G*G*G*T*C*T*T*G*A*A*A*+ | 90227 | 19 |
| | AATG | | A*+T*+G | | |
| 3639 | AGTGCTGGGTCTTGA | A573637H | +A*+G*+T*G*C*T*G*G*G*T*C*T*T*G*A*A*+ | 90228 | 19 |
| | AAAT | | A*+A*+T | | |
| 3640 | CAGTGCTGGGTCTTG | A573638H | +C*+A*+G*T*G*C*T*G*G*G*T*C*T*T*G*A*+ | 90229 | 19 |
| | AAAA | | A*+A*+A | | |
| 3641 | GCAGTGCTGGGTCTT | A573639H | +G*+C*+A*G*T*G*C*T*G*G*G*T*C*T*T*G*+ | 90230 | 19 |
| | GAAA | | A*+A*+A | | |
| 3642 | CTGCAGTGCTGGGTC | A573640H | +C*+T*+G*C*A*G*T*G*C*T*G*G*G*T*C*T*+ | 90232 | 19 |
| | TTGA | | T*+G*+A | | |
| 3643 | TCACTTAACTGCAGT | A573641H | +T*+C*+A*C*T*T*A*A*C*T*G*C*A*G*T*G*+ | 90240 | 19 |
| | GCTG | | C*+T*+G | | |
| 3644 | AGTCACTTAACTGCA | A573642H | +A*+G*+T*C*A*C*T*T*A*A*C*T*G*C*A*G*+ | 90242 | 19 |
| | GTGC | | T*+G*+C | | |
| 3645 | GCCACTTGGGTTCCA | A573643H | +G*+C*+C*A*C*T*T*G*G*G*T*T*C*C*A*C*+ | 90266 | 19 |
| | CAAA | | A*+A*+A | | |
| 3646 | GCCACTTGGGTTCCA | A573644H | +G*+C*+C*A*C*T*T*G*G*G*T*T*C*C*A*+C* | 90267 | 18 |
| | CAA | | +A*+A | | |
| 3647 | AGCCACTTGGGTTCC | A573645H | +A*+G*+C*C*A*C*T*T*G*G*G*T*T*C*C*A*+ | 90267 | 19 |
| | ACAA | | C*+A*+A | | |
| 3648 | AAAGCCACTTGGGTT | A573646H | +A*+A*+A*G*C*C*A*C*T*T*G*G*G*T*T*C*+ | 90269 | 19 |
| | CCAC | | C*+A*+C | | |
| 3649 | CAAAGCCACTTGGGT | A573647H | +C*+A*+A*A*G*C*C*A*C*T*T*G*G*G*T*T*+ | 90270 | 19 |
| | TCCA | | C*+C*+A | | |
| 3650 | TTACAAAGCCACTTG | A573648H | +T*+T*+A*C*A*A*A*G*C*C*A*C*T*T*G*G*+ | 90273 | 19 |
| | GGTT | | G*+T*+T | | |
| 3651 | CCATGATTTTACAAA | A573649H | +C*+C*+A*T*G*A*T*T*T*T*A*C*A*A*A*G*+ | 90281 | 19 |
| | GCCA | | C*+C*+A | | |
| 3652 | GCCATGATTTTACAA | A573650H | +G*+C*+C*A*T*G*A*T*T*T*T*A*C*A*A*A*+ | 90282 | 19 |
| | AGCC | | G*+C*+C | | |
| 3653 | ATCTGTTCTATGCTTT | A573651Hi | +A*+T*+C*T*G*T*T*C*T*A*T*G*C*T*T*T*+ | 90341 | 19 |
| | GAC | | G*+A*+C | | |
| 3654 | CATCTGTTCTATGCTT | A573652Hi | +C*+A*+T*C*T*G*T*T*C*T*A*T*G*C*T*T*+ | 90342 | 19 |
| | TGA | | T*+G*+A | | |
| 3655 | CCATCTGTTCTATGC | A573653Hi | +C*+C*+A*T*C*T*G*T*T*C*T*A*T*G*C*T*+ | 90343 | 19 |
| | TTTG | | T*+T*+G | | |
| 3656 | GTTTCTGCCTTAACT | A573654Hi | +G*+T*+T*T*C*T*G*C*C*T*T*A*A*C*T*A*+ | 90393 | 19 |
| | AAAC | | A*+A*+C | | |
| 3657 | ATGTGAAATGTTCCA | A573655Hi | +A*+T*+G*T*G*A*A*A*T*G*T*T*C*C*A*C*+ | 90429 | 19 |
| | CAGT | | A*+G*+T | | |
| 3658 | CTATGTGAAATGTTC | A573656Hi | +C*+T*+A*T*G*T*G*A*A*A*T*G*T*T*C*C*+ | 90431 | 19 |
| | CACA | | A*+C*+A | | |
| 3659 | TAGTCTTCTATGTGA | A573657Hi | +T*+A*+G*T*C*T*T*C*T*A*T*G*T*G*A*A*+ | 90438 | 19 |
| | AATG | | A*+T*+G | | |
| 3660 | CTTTCCTTCTTCCATG | A573658Hi | +C*+T*+T*T*C*C*T*T*C*T*T*C*C*A*T*G*+ | 90466 | 19 |
| | AAG | | A*+A*+G | | |
| 3661 | CATGGAAGTGAACTC | A573659Hi | +C*+A*+T*G*G*A*A*G*T*G*A*A*C*T*C*A*+ | 90488 | 19 |
| | AAGA | | A*+G*+A | | |
| 3662 | GTCATGGAAGTGAAC | A573660Hi | +G*+T*+C*A*T*G*G*A*A*G*T*G*A*A*C*+T* | 90491 | 18 |
| | TCA | | +C*+A | | |
| 3663 | AAGTCATGGAAGTGA | A573661Hi | +A*+A*+G*T*C*A*T*G*G*A*A*G*T*G*A*A*+ | 90492 | 19 |
| | ACTC | | C*+T*+C | | |
| 3664 | TACAAACATCTGTAT | A573662Hi | +T*+A*+C*A*A*A*C*A*T*C*T*G*T*A*T*G*+ | 90521 | 19 |
| | GTGG | | T*+G*+G | | |
| 3665 | TCTAGTAAAGCAGCA | A573663Hi | +T*+C*+T*A*G*T*A*A*A*G*C*A*G*C*A*+T* | 90550 | 18 |
| | TGG | | +G*+G | | |
| 3666 | AGGATCTAGTAAAGC | A573664Hi | +A*+G*+G*A*T*C*T*A*G*T*A*A*A*G*C*A*+ | 90553 | 19 |
| | AGCA | | G*+C*+A | | |
| 3667 | CATCAGGATCTAGTA | A573665Hi | +C*+A*+T*C*A*G*G*A*T*C*T*A*G*T*A*A*+ | 90557 | 19 |
| | AAGC | | A*+G*+C | | |
| 3668 | TCATCAGGATCTAGT | A573666Hi | +T*+C*+A*T*C*A*G*G*A*T*C*T*A*G*T*A*+ | 90558 | 19 |
| | AAAG | | A*+A*+G | | |
| 3669 | ACATCATCAGGATCT | A573667Hi | +A*+C*+A*T*C*A*T*C*A*G*G*A*T*C*T*A*+ | 90561 | 19 |
| | AGTA | | G*+T*+A | | |
| 3670 | TGACATCATCAGGAT | A573668Hi | +T*+G*+A*C*A*T*C*A*T*C*A*G*G*A*T*C*+ | 90563 | 19 |
| | CTAG | | T*+A*+G | | |
| 3671 | GTTTCACAGGCTCAG | A573669Hi | +G*+T*+T*T*C*A*C*A*G*G*C*T*C*A*G*C*+ | 90660 | 19 |
| | CAAC | | A*+A*+C | | |
| 3672 | AGGTTTCACAGGCTC | A573670Hi | +A*+G*+G*T*T*T*C*A*C*A*G*G*C*T*C*A*+ | 90662 | 19 |
| | AGCA | | G*+C*+A | | |
| 3673 | GATAGAGGTTTCACA | A573671Hi | +G*+A*+T*A*G*A*G*G*T*T*T*C*A*C*A*G*+ | 90667 | 19 |
| | GGCT | | G*+C*+T | | |
| 3674 | CTAGATAGAGGTTTC | A573672Hi | +C*+T*+A*G*A*T*A*G*A*G*G*T*T*T*C*A*+ | 90670 | 19 |
| | ACAG | | C*+A*+G | | |
| 3675 | CTAGATAGAGGTTTC | A573673Hi | +C*+T*+A*G*A*T*A*G*A*G*G*T*T*T*C*+A* | 90671 | 18 |
| | ACA | | +C*+A | | |
| 3676 | TCTAGATAGAGGTTT | A573674Hi | +T*+C*+T*A*G*A*T*A*G*A*G*G*T*T*T*C*+ | 90671 | 19 |
| | CACA | | A*+C*+A | | |
| 3677 | ATCTAGATAGAGGTT | A573675Hi | +A*+T*+C*T*A*G*A*T*A*G*A*G*G*T*T*T*+ | 90672 | 19 |
| | TCAC | | C*+A*+C | | |
| 3678 | TATCTAGATAGAGGT | A573676Hi | +T*+A*+T*C*T*A*G*A*T*A*G*A*G*G*T*T*+ | 90673 | 19 |
| | TTCA | | T*+C*+A | | |
| 3679 | ATGTTATCTAGATAG | A573677Hi | +A*+T*+G*T*T*A*T*C*T*A*G*A*T*A*G*A*+ | 90677 | 19 |
| | AGGT | | G*+G*+T | | |
| 3680 | ATGTTATCTAGATAG | A573678Hi | +A*+T*+G*T*T*A*T*C*T*A*G*A*T*A*G*+A* | 90678 | 18 |
| | AGG | | +G*+G | | |
| 3681 | CATGTTATCTAGATA | A573679Hi | +C*+A*+T*G*T*T*A*T*C*T*A*G*A*T*A*G*+ | 90678 | 19 |
| | GAGG | | A*+G*+G | | |
| 3682 | CATGTTATCTAGATA | A573680Hi | +C*+A*+T*G*T*T*A*T*C*T*A*G*A*T*A*+G* | 90679 | 18 |
| | GAG | | +A*+G | | |
| 3683 | CCATGTTATCTAGAT | A573681Hi | +C*+C*+A*T*G*T*T*A*T*C*T*A*G*A*T*A*+ | 90679 | 19 |
| | AGAG | | G*+A*+G | | |
| 3684 | TTATGTCAAAGCTAC | A573682Hi | +T*+T*+A*T*G*T*C*A*A*A*G*C*T*A*C*A*+ | 90869 | 19 |
| | AGAG | | G*+A*+G | | |
| 3685 | CAATGTGCAACAAAG | A573683Hi | +C*+A*+A*T*G*T*G*C*A*A*C*A*A*A*G*A*+ | 90958 | 19 |
| | AATT | | A*+T*+T | | |
| 3686 | ACAATGTGCAACAAA | A573684Hi | +A*+C*+A*A*T*G*T*G*C*A*A*C*A*A*A*G*+ | 90959 | 19 |
| | GAAT | | A*+A*+T | | |
| 3687 | TCACATTCTCCAAGC | A573685Hi | +T*+C*+A*C*A*T*T*C*T*C*C*A*A*G*C*A*+ | 91070 | 19 |
| | AATA | | A*+T*+A | | |
| 3688 | TATGGTTTAATTGGA | A573686Hi | +T*+A*+T*G*G*T*T*T*A*A*T*T*G*G*A*A*+ | 91206 | 19 |
| | AGAG | | G*+A*+G | | |
| 3689 | TATGGTTTAATTGGA | A573687Hi | +T*+A*+T*G*G*T*T*T*A*A*T*T*G*G*A*+A* | 91207 | 18 |
| | AGA | | +G*+A | | |
| 3690 | CTTTATGGTTTAATTG | A573688Hi | +C*+T*+T*T*A*T*G*G*T*T*T*A*A*T*T*G*+ | 91209 | 19 |
| | GAA | | G*+A*+A | | |
| 3691 | TAATTACCTTTATGGT | A573689Hi | +T*+A*+A*T*T*A*C*C*T*T*T*A*T*G*G*T*+ | 91216 | 19 |
| | TTA | | T*+T*+A | | |
| 3692 | TGAGTGAATTTAATT | A573690Hi | +T*+G*+A*G*T*G*A*A*T*T*T*A*A*T*T*+A* | 91227 | 18 |
| | ACC | | +C*+C | | |
| 3693 | TAGATGGAATTTAGG | A573691Hi | +T*+A*+G*A*T*G*G*A*A*T*T*T*A*G*G*A*+ | 91261 | 19 |
| | AACA | | A*+C*+A | | |
| 3694 | GCATTAAGAAATTTC | A573692Hi | +G*+C*+A*T*T*A*A*G*A*A*A*T*T*T*C*A*+ | 91570 | 19 |
| | ACAG | | C*+A*+G | | |
| 3695 | TAATGCATTAAGAAA | A573693Hi | +T*+A*+A*T*G*C*A*T*T*A*A*G*A*A*A*T*+ | 91574 | 19 |
| | TTTC | | T*+T*+C | | |
| 3696 | GCAGCTACCTACACA | A573694Hi | +G*+C*+A*G*C*T*A*C*C*T*A*C*A*C*A*T*+ | 91811 | 19 |
| | TCTT | | C*+T*+T | | |
| 3697 | CAGGATGCAGCTACC | A573695Hi | +C*+A*+G*G*A*T*G*C*A*G*C*T*A*C*C*T*+ | 91817 | 19 |
| | TACA | | A*+C*+A | | |
| 3698 | AGGATGCAGCTACCT | A573696Hi | +A*+G*+G*A*T*G*C*A*G*C*T*A*C*C*T*+A* | 91817 | 18 |
| | ACA | | +C*+A | | |
| 3699 | GTACAGGATGCAGCT | A573697Hi | +G*+T*+A*C*A*G*G*A*T*G*C*A*G*C*T*A*+ | 91820 | 19 |
| | ACCT | | C*+C*+T | | |
| 3700 | TACAGGATGCAGCTA | A573698Hi | +T*+A*+C*A*G*G*A*T*G*C*A*G*C*T*A*+C* | 91820 | 18 |
| | CCT | | +C*+T | | |
| 3701 | TGTACAGGATGCAGC | A573699Hi | +T*+G*+T*A*C*A*G*G*A*T*G*C*A*G*C*T*+ | 91821 | 19 |
| | TACC | | A*+C*+C | | |
| 3702 | GTACAGGATGCAGCT | A573700Hi | +G*+T*+A*C*A*G*G*A*T*G*C*A*G*C*T*+A* | 91821 | 18 |
| | ACC | | +C*+C | | |
| 3703 | CTTTGCTGTACAGGA | A573701Hi | +C*+T*+T*T*G*C*T*G*T*A*C*A*G*G*A*T*+ | 91827 | 19 |
| | TGCA | | G*+C*+A | | |
| 3704 | TCCTTTGCTGTACAG | A573702Hi | +T*+C*+C*T*T*T*G*C*T*G*T*A*C*A*G*G*+ | 91829 | 19 |
| | GATG | | A*+T*+G | | |
| 3705 | TGGAGTTGAGTTACC | A573703Hi | +T*+G*+G*A*G*T*T*G*A*G*T*T*A*C*C*T*+ | 91902 | 19 |
| | TCCT | | C*+C*+T | | |
| 3706 | GCAAACCTCTTCCTT | A573704Hi | +G*+C*+A*A*A*C*C*T*C*T*T*C*C*T*T*A*+ | 92034 | 19 |
| | ATTC | | T*+T*+C | | |
| 3707 | TGATGCAAACCTCTT | A573705Hi | +T*+G*+A*T*G*C*A*A*A*C*C*T*C*T*T*C*+ | 92038 | 19 |
| | CCTT | | C*+T*+T | | |
| 3708 | TATTGCAGTCATAGT | A573706Hi | +T*+A*+T*T*G*C*A*G*T*C*A*T*A*G*T*G*+ | 92084 | 19 |
| | GAAA | | A*+A*+A | | |
| 3709 | TATTGCAGTCATAGT | A573707Hi | +T*+A*+T*T*G*C*A*G*T*C*A*T*A*G*T*+G* | 92085 | 18 |
| | GAA | | +A*+A | | |
| 3710 | TTATTGCAGTCATAG | A573708Hi | +T*+T*+A*T*T*G*C*A*G*T*C*A*T*A*G*T*+ | 92085 | 19 |
| | TGAA | | G*+A*+A | | |
| 3711 | TTATTGCAGTCATAG | A573709Hi | +T*+T*+A*T*T*G*C*A*G*T*C*A*T*A*G*+T* | 92086 | 18 |
| | TGA | | +G*+A | | |
| 3712 | TTTATTGCAGTCATA | A573710Hi | +T*+T*+T*A*T*T*G*C*A*G*T*C*A*T*A*+G* | 92087 | 18 |
| | GTG | | +T*+G | | |
| 3713 | TTCAACTTAATTACTC | A573711Hi | +T*+T*+C*A*A*C*T*T*A*A*T*T*A*C*T*+C* | 92121 | 18 |
| | CA | | +C*+A | | |
| 3714 | TTTCAACTTAATTACT | A573712Hi | +T*+T*+T*C*A*A*C*T*T*A*A*T*T*A*C*T*+ | 92121 | 19 |
| | CCA | | C*+C*+A | | |
| 3715 | AGTGGCTCAAATTAA | A573713Hi | +A*+G*+T*G*G*C*T*C*A*A*A*T*T*A*A*C*+ | 92197 | 19 |
| | CTTA | | T*+T*+A | | |
| 3716 | TTGAGTGGCTCAAAT | A573714Hi | +T*+T*+G*A*G*T*G*G*C*T*C*A*A*A*T*T*+ | 92200 | 19 |
| | TAAC | | A*+A*+C | | |
| 3717 | AGTAACTTTGAGTGG | A573715Hi | +A*+G*+T*A*A*C*T*T*T*G*A*G*T*G*G*+C* | 92208 | 18 |
| | CTC | | +T*+C | | |
| 3718 | AAGTAACTTTGAGTG | A573716Hi | +A*+A*+G*T*A*A*C*T*T*T*G*A*G*T*G*+G* | 92209 | 18 |
| | GCT | | +C*+T | | |
| 3719 | GCTCTTGGTTTCACT | A573717Hi | +G*+C*+T*C*T*T*G*G*T*T*T*C*A*C*T*T*+ | 92376 | 19 |
| | TGAT | | G*+A*+T | | |
| 3720 | TGCTCTTGGTTTCAC | A573718Hi | +T*+G*+C*T*C*T*T*G*G*T*T*T*C*A*C*T*+ | 92377 | 19 |
| | TTGA | | T*+G*+A | | |
| 3721 | AGTTGCTCTTGGTTT | A573719Hi | +A*+G*+T*T*G*C*T*C*T*T*G*G*T*T*T*C*+ | 92380 | 19 |
| | CACT | | A*+C*+T | | |
| 3722 | CAAGTTGCTCTTGGT | A573720Hi | +C*+A*+A*G*T*T*G*C*T*C*T*T*G*G*T*T*+ | 92382 | 19 |
| | TTCA | | T*+C*+A | | |
| 3723 | TGTCAGACAAGTTGC | A573721Hi | +T*+G*+T*C*A*G*A*C*A*A*G*T*T*G*C*T*+ | 92389 | 19 |
| | TCTT | | C*+T*+T | | |
| 3724 | CTGTCAGACAAGTTG | A573722Hi | +C*+T*+G*T*C*A*G*A*C*A*A*G*T*T*G*C*+ | 92390 | 19 |
| | CTCT | | T*+C*+T | | |
| 3725 | CTGTCAGACAAGTTG | A573723Hi | +C*+T*+G*T*C*A*G*A*C*A*A*G*T*T*G*+C* | 92391 | 18 |
| | CTC | | +T*+C | | |
| 3726 | GCTGTCAGACAAGTT | A573724Hi | +G*+C*+T*G*T*C*A*G*A*C*A*A*G*T*T*G*+ | 92391 | 19 |
| | GCTC | | C*+T*+C | | |
| 3727 | ATTCATGAAGGTGAA | A573725Hi | +A*+T*+T*C*A*T*G*A*A*G*G*T*G*A*A*T*+ | 92430 | 19 |
| | TACT | | A*+C*+T | | |
| 3728 | TGATTCATGAAGGTG | A573726Hi | +T*+G*+A*T*T*C*A*T*G*A*A*G*G*T*G*A*+ | 92432 | 19 |
| | AATA | | A*+T*+A | | |
| 3729 | CTTGATTCATGAAGG | A573727Hi | +C*+T*+T*G*A*T*T*C*A*T*G*A*A*G*G*T*+ | 92434 | 19 |
| | TGAA | | G*+A*+A | | |
| 3730 | GCCTTGATTCATGAA | A573728Hi | +G*+C*+C*T*T*G*A*T*T*C*A*T*G*A*A*G*+ | 92436 | 19 |
| | GGTG | | G*+T*+G | | |
| 3731 | GCCTTGATTCATGAA | A573729Hi | +G*+C*+C*T*T*G*A*T*T*C*A*T*G*A*A*+G* | 92437 | 18 |
| | GGT | | +G*+T | | |
| 3732 | TTGCCTTGATTCATG | A573730Hi | +T*+T*+G*C*C*T*T*G*A*T*T*C*A*T*G*A*+ | 92438 | 19 |
| | AAGG | | A*+G*+G | | |
| 3733 | TTCTGCACCATGAAA | A573731Hi | +T*+T*+C*T*G*C*A*C*C*A*T*G*A*A*A*T*+ | 92465 | 19 |
| | TTAT | | T*+A*+T | | |
| 3734 | CTTCTGCACCATGAA | A573732Hi | +C*+T*+T*C*T*G*C*A*C*C*A*T*G*A*A*A*+ | 92466 | 19 |
| | ATTA | | T*+T*+A | | |
| 3735 | TAGAGCTTCTGCACC | A573733Hi | +T*+A*+G*A*G*C*T*T*C*T*G*C*A*C*C*A*+ | 92471 | 19 |
| | ATGA | | T*+G*+A | | |
| 3736 | TTAGAGCTTCTGCAC | A573734Hi | +T*+T*+A*G*A*G*C*T*T*C*T*G*C*A*C*C*+ | 92472 | 19 |
| | CATG | | A*+T*+G | | |
| 3737 | CATTAGAGCTTCTGC | A573735Hi | +C*+A*+T*T*A*G*A*G*C*T*T*C*T*G*C*A*+ | 92474 | 19 |
| | ACCA | | C*+C*+A | | |
| 3738 | CATTAGAGCTTCTGC | A573736Hi | +C*+A*+T*T*A*G*A*G*C*T*T*C*T*G*C*+A* | 92475 | 18 |
| | ACC | | +C*+C | | |
| 3739 | CTCATTAGAGCTTCT | A573737Hi | +C*+T*+C*A*T*T*A*G*A*G*C*T*T*C*T*G*+ | 92476 | 19 |
| | GCAC | | C*+A*+C | | |
| 3740 | CCTGCCTTAATGGTA | A573738Hi | +C*+C*+T*G*C*C*T*T*A*A*T*G*G*T*A*+C* | 92547 | 18 |
| | CCT | | +C*+T | | |
| 3741 | AGTTAAATGAGTCTT | A573739Hi | +A*+G*+T*T*A*A*A*T*G*A*G*T*C*T*T*G*+ | 92572 | 19 |
| | GTAA | | T*+A*+A | | |
| 3742 | TTTATCATACAGTTAA | A573740Hi | +T*+T*+T*A*T*C*A*T*A*C*A*G*T*T*A*A*+ | 92582 | 19 |
| | ATG | | A*+T*+G | | |
| 3743 | ATGTTCCACCTGATT | A573741Hi | +A*+T*+G*T*T*C*C*A*C*C*T*G*A*T*T*A*+ | 92624 | 19 |
| | AATT | | A*+T*+T | | |
| 3744 | CTTATTCAAGGAAAG | A573742Hi | +C*+T*+T*A*T*T*C*A*A*G*G*A*A*A*G*A*+ | 92711 | 19 |
| | AGAA | | G*+A*+A | | |
| 3745 | GCCTTATTCAAGGAA | A573743Hi | +G*+C*+C*T*T*A*T*T*C*A*A*G*G*A*A*A*+ | 92713 | 19 |
| | AGAG | | G*+A*+G | | |
| 3746 | AACTGATGCCTTATT | A573744Hi | +A*+A*+C*T*G*A*T*G*C*C*T*T*A*T*T*C*+ | 92720 | 19 |
| | CAAG | | A*+A*+G | | |
| 3747 | ACCTGAATAGCTAAC | A573745Hi | +A*+C*+C*T*G*A*A*T*A*G*C*T*A*A*C*T*+ | 92732 | 19 |
| | TGAT | | G*+A*+T | | |
| 3748 | ACCTGAATAGCTAAC | A573746Hi | +A*+C*+C*T*G*A*A*T*A*G*C*T*A*A*C*+T* | 92733 | 18 |
| | TGA | | +G*+A | | |
| 3749 | TAACCTGAATAGCTA | A573747Hi | +T*+A*+A*C*C*T*G*A*A*T*A*G*C*T*A*A*+ | 92734 | 19 |
| | ACTG | | C*+T*+G | | |
| 3750 | TGACTTTATCACCTTT | A573748Hi | +T*+G*+A*C*T*T*T*A*T*C*A*C*C*T*T*T*+ | 92780 | 19 |
| | AAA | | A*+A*+A | | |
| 3751 | TATCTTGACTTTATCA | A573749Hi | +T*+A*+T*C*T*T*G*A*C*T*T*T*A*T*C*+A* | 92786 | 18 |
| | CC | | +C*+C | | |
| 3752 | CAACATTATCTTGAC | A573750Hi | +C*+A*+A*C*A*T*T*A*T*C*T*T*G*A*C*T*+ | 92791 | 19 |
| | TTTA | | T*+T*+A | | |
| 3753 | TCTGCAACATTATCTT | A573751Hi | +T*+C*+T*G*C*A*A*C*A*T*T*A*T*C*T*T*+ | 92795 | 19 |
| | GAC | | G*+A*+C | | |
| 3754 | AATCTGCAACATTAT | A573752Hi | +A*+A*+T*C*T*G*C*A*A*C*A*T*T*A*T*C*+ | 92797 | 19 |
| | CTTG | | T*+T*+G | | |
| 3755 | AATGATAAATTGTTC | A573753Hi | +A*+A*+T*G*A*T*A*A*A*T*T*G*T*T*C*A*+ | 92859 | 19 |
| | ACAT | | C*+A*+T | | |
| 3756 | GTGGAGCAGAACCAC | A573754Hi | +G*+T*+G*G*A*G*C*A*G*A*A*C*C*A*C*G* | 92909 | 19 |
| | GCAG | | +C*+A*+G | | |
| 3757 | CATGTGGAGCAGAAC | A573755Hi | +C*+A*+T*G*T*G*G*A*G*C*A*G*A*A*C*C*+ | 92912 | 19 |
| | CACG | | A*+C*+G | | |
| 3758 | GTACCTTTCTTGCAC | A573756Hi | +G*+T*+A*C*C*T*T*T*C*T*T*G*C*A*C*T*+ | 92978 | 19 |
| | TTTC | | T*+T*+C | | |
| 3759 | ATGGAGAAGCACCAA | A573757Hi | +A*+T*+G*G*A*G*A*A*G*C*A*C*C*A*A*T*+ | 93011 | 19 |
| | TACT | | A*+C*+T | | |
| 3760 | GGAGAAGCACCAATA | A573758Hi | +G*+G*+A*G*A*A*G*C*A*C*C*A*A*T*+A*+ | 93011 | 17 |
| | CT | | C*+T | | |
| 3761 | TGGAGAAGCACCAAT | A573759Hi | +T*+G*+G*A*G*A*A*G*C*A*C*C*A*A*T*+A* | 93011 | 18 |
| | ACT | | +C*+T | | |
| 3762 | CATGGAGAAGCACCA | A573760Hi | +C*+A*+T*G*G*A*G*A*A*G*C*A*C*C*A*A*+ | 93012 | 19 |
| | ATAC | | T*+A*+C | | |
| 3763 | ATGGAGAAGCACCAA | A573761Hi | +A*+T*+G*G*A*G*A*A*G*C*A*C*C*A*A*+T* | 93012 | 18 |
| | TAC | | +A*+C | | |
| 3764 | AACCAGCCACCATGA | A573762Hi | +A*+A*+C*C*A*G*C*C*A*C*C*A*T*G*A*+A* | 93094 | 18 |
| | AAG | | +A*+G | | |
| 3765 | TTCTCTTGGTAACCA | A573763Hi | +T*+T*+C*T*C*T*T*G*G*T*A*A*C*C*A*G*+ | 93103 | 19 |
| | GCCA | | C*+C*+A | | |
| 3766 | TTTCTCTTGGTAACC | A573764Hi | +T*+T*+T*C*T*C*T*T*G*G*T*A*A*C*C*A*+ | 93104 | 19 |
| | AGCC | | G*+C*+C | | |
| 3767 | ATTTCTCTTGGTAAC | A573765Hi | +A*+T*+T*T*C*T*C*T*T*G*G*T*A*A*C*C*+ | 93105 | 19 |
| | CAGC | | A*+G*+C | | |
| 3768 | TTCATTTCTCTTGGTA | A573766Hi | +T*+T*+C*A*T*T*T*C*T*C*T*T*G*G*T*A*+ | 93108 | 19 |
| | ACC | | A*+C*+C | | |
| 3769 | CAAAGATGGACACAT | A573767Hi | +C*+A*+A*A*G*A*T*G*G*A*C*A*C*A*T*C*+ | 93282 | 19 |
| | CATT | | A*+T*+T | | |
| 3770 | GAAGTTTCCAAAGAT | A573768Hi | +G*+A*+A*G*T*T*T*C*C*A*A*A*G*A*T*G*+ | 93290 | 19 |
| | GGAC | | G*+A*+C | | |
| 3771 | ATTATTATCTAGTTAT | A573769Hi | +A*+T*+T*A*T*T*A*T*C*T*A*G*T*T*A*T*+ | 93399 | 19 |
| | TCT | | T*+C*+T | | |
| 3772 | CATTATTATCTAGTTA | A573770Hi | +C*+A*+T*T*A*T*T*A*T*C*T*A*G*T*T*A*+ | 93400 | 19 |
| | TTC | | T*+T*+C | | |
| 3773 | GTTAGCCATTATTAT | A573771Hi | +G*+T*+T*A*G*C*C*A*T*T*A*T*T*A*T*+C* | 93407 | 18 |
| | CTA | | +T*+A | | |
| 3774 | TGTTAGCCATTATTAT | A573772Hi | +T*+G*+T*T*A*G*C*C*A*T*T*A*T*T*A*T*+ | 93407 | 19 |
| | CTA | | C*+T*+A | | |
| 3775 | ATTCAACCTCTCTGT | A573773Hi | +A*+T*+T*C*A*A*C*C*T*C*T*C*T*G*T*C*+ | 93531 | 19 |
| | CAAT | | A*+A*+T | | |
| 3776 | CTCTTCCTGAATGTG | A573774Hi | +C*+T*+C*T*T*C*C*T*G*A*A*T*G*T*G*T*+ | 93562 | 19 |
| | TACC | | A*+C*+C | | |
| 3777 | CCTCTTCCTGAATGT | A573775Hi | +C*+C*+T*C*T*T*C*C*T*G*A*A*T*G*T*G*+ | 93563 | 19 |
| | GTAC | | T*+A*+C | | |
| 3778 | AACTCTACCTCTTCCT | A573776Hi | +A*+A*+C*T*C*T*A*C*C*T*C*T*T*C*C*T*+ | 93570 | 19 |
| | GAA | | G*+A*+A | | |
| 3779 | TTTAAGTGCTAAGAC | A573777Hi | +T*+T*+T*A*A*G*T*G*C*T*A*A*G*A*C*C*+ | 93754 | 19 |
| | CTTT | | T*+T*+T | | |
| 3780 | GTACTTTATAGCAAT | A573778Hi | +G*+T*+A*C*T*T*T*A*T*A*G*C*A*A*T*T*+ | 93826 | 19 |
| | TCCA | | C*+C*+A | | |
| 3781 | TGTACTTTATAGCAA | A573779Hi | +T*+G*+T*A*C*T*T*T*A*T*A*G*C*A*A*T*+ | 93827 | 19 |
| | TTCC | | T*+C*+C | | |
| 3782 | GTGTACTTTATAGCA | A573780Hi | +G*+T*+G*T*A*C*T*T*T*A*T*A*G*C*A*A*+ | 93828 | 19 |
| | ATTC | | T*+T*+C | | |
| 3783 | CATATTGGTATTTAAT | A573781Hi | +C*+A*+T*A*T*T*G*G*T*A*T*T*T*A*A*T*+ | 93903 | 19 |
| | GAC | | G*+A*+C | | |
| 3784 | GTTATAAAGTCCTTA | A573782Hi | +G*+T*+T*A*T*A*A*A*G*T*C*C*T*T*A*G*+ | 94004 | 19 |
| | GGCC | | G*+C*+C | | |
| 3785 | GTTATAAAGTCCTTA | A573783Hi | +G*+T*+T*A*T*A*A*A*G*T*C*C*T*T*A*+G* | 94005 | 18 |
| | GGC | | +G*+C | | |
| 3786 | GAAATACCAGGTTAT | A573784Hi | +G*+A*+A*A*T*A*C*C*A*G*G*T*T*A*T*A*+ | 94014 | 19 |
| | AAAG | | A*+A*+G | | |
| 3787 | GTGAAATACCAGGTT | A573785Hi | +G*+T*+G*A*A*A*T*A*C*C*A*G*G*T*T*+A* | 94017 | 18 |
| | ATA | | +T*+A | | |
| 3788 | TTCTACTTGCAGTGT | A573786Hi | +T*+T*+C*T*A*C*T*T*G*C*A*G*T*G*T*C*+ | 94478 | 19 |
| | CTGG | | T*+G*+G | | |
| 3789 | GGATTCTACTTGCAG | A573787Hi | +G*+G*+A*T*T*C*T*A*C*T*T*G*C*A*G*+T* | 94482 | 18 |
| | TGT | | +G*+T | | |
| 3790 | GATGGATTCTACTTG | A573788Hi | +G*+A*+T*G*G*A*T*T*C*T*A*C*T*T*G*C*+ | 94484 | 19 |
| | CAGT | | A*+G*+T | | |
| 3791 | ATGGATTCTACTTGC | A573789Hi | +A*+T*+G*G*A*T*T*C*T*A*C*T*T*G*C*+A* | 94484 | 18 |
| | AGT | | +G*+T | | |
| 3792 | TGATGGATTCTACTT | A573790Hi | +T*+G*+A*T*G*G*A*T*T*C*T*A*C*T*T*G*+ | 94485 | 19 |
| | GCAG | | C*+A*+G | | |
| 3793 | GATGGATTCTACTTG | A573791Hi | +G*+A*+T*G*G*A*T*T*C*T*A*C*T*T*G*+C* | 94485 | 18 |
| | CAG | | +A*+G | | |
| 3794 | GATTCTCAAAGTAAC | A573792Hi | +G*+A*+T*T*C*T*C*A*A*A*G*T*A*A*C*+T* | 94511 | 18 |
| | TAC | | +A*+C | | |
| 3795 | TCATGATTCTCAAAG | A573793Hi | +T*+C*+A*T*G*A*T*T*C*T*C*A*A*A*G*T*+ | 94514 | 19 |
| | TAAC | | A*+A*+C | | |
| 3796 | TTTCATGATTCTCAAA | A573794Hi | +T*+T*+T*C*A*T*G*A*T*T*C*T*C*A*A*A*+ | 94516 | 19 |
| | GTA | | G*+T*+A | | |
| 3797 | GAGTTTCATGATTCT | A573795Hi | +G*+A*+G*T*T*T*C*A*T*G*A*T*T*C*T*C*+ | 94519 | 19 |
| | CAAA | | A*+A*+A | | |
| 3798 | AGAGTTTCATGATTC | A573796Hi | +A*+G*+A*G*T*T*T*C*A*T*G*A*T*T*C*T*+ | 94520 | 19 |
| | TCAA | | C*+A*+A | | |
| 3799 | ATTTATATACTAGGA | A573797Hi | +A*+T*+T*T*A*T*A*T*A*C*T*A*G*G*A*G*+ | 94571 | 19 |
| | GAGG | | A*+G*+G | | |
| 3800 | CATTTATATACTAGG | A573798Hi | +C*+A*+T*T*T*A*T*A*T*A*C*T*A*G*G*A*+ | 94572 | 19 |
| | AGAG | | G*+A*+G | | |
| 3801 | ACAGGTTTGAAATAG | A573799Hi | +A*+C*+A*G*G*T*T*T*G*A*A*A*T*A*G*A*+ | 94600 | 19 |
| | AAAT | | A*+A*+T | | |
| 3802 | CAGTCTCATCAATAA | A573800Hi | +C*+A*+G*T*C*T*C*A*T*C*A*A*T*A*A*T*+ | 94654 | 19 |
| | TATA | | A*+T*+A | | |
| 3803 | GCAGTCTCATCAATA | A573801Hi | +G*+C*+A*G*T*C*T*C*A*T*C*A*A*T*A*A*+ | 94655 | 19 |
| | ATAT | | T*+A*+T | | |
| 3804 | GCAGTCTCATCAATA | A573802Hi | +G*+C*+A*G*T*C*T*C*A*T*C*A*A*T*A*+A* | 94656 | 18 |
| | ATA | | +T*+A | | |
| 3805 | GTCTCTTTGCTCAGT | A573803Hi | +G*+T*+C*T*C*T*T*T*G*C*T*C*A*G*T*G*+ | 94902 | 19 |
| | GAAA | | A*+A*+A | | |
| 3806 | GTCTCTTTGCTCAGT | A573804Hi | +G*+T*+C*T*C*T*T*T*G*C*T*C*A*G*T*+G* | 94903 | 18 |
| | GAA | | +A*+A | | |
| 3807 | GTTGCAAATTTAAAG | A573805Hi | +G*+T*+T*G*C*A*A*A*T*T*T*A*A*A*G*C*+ | 94925 | 19 |
| | CATT | | A*+T*+T | | |
| 3808 | CCACTTTCACTGTCA | A573806H | +C*+C*+A*C*T*T*T*C*A*C*T*G*T*C*A*A*+ | 94992 | 19 |
| | AAAT | | A*+A*+T | | |
| 3809 | CTTCCACTTTCACTGT | A573807H | +C*+T*+T*C*C*A*C*T*T*T*C*A*C*T*G*T*+ | 94995 | 19 |
| | CAA | | C*+A*+A | | |
| 3810 | CAACCTTCCACTTTC | A573808H | +C*+A*+A*C*C*T*T*C*C*A*C*T*T*T*C*A*+ | 94999 | 19 |
| | ACTG | | C*+T*+G | | |
| 3811 | GACAACCTTCCACTT | A573809H | +G*+A*+C*A*A*C*C*T*T*C*C*A*C*T*T*T*+ | 95001 | 19 |
| | TCAC | | C*+A*+C | | |
| 3812 | GGACAACCTTCCACT | A573810H | +G*+G*+A*C*A*A*C*C*T*T*C*C*A*C*T*T*+ | 95002 | 19 |
| | TTCA | | T*+C*+A | | |
| 3813 | TTGGACAACCTTCCA | A573811H | +T*+T*+G*G*A*C*A*A*C*C*T*T*C*C*A*C*+ | 95004 | 19 |
| | CTTT | | T*+T*+T | | |
| 3814 | TTGGACAACCTTCCA | A573812H | +T*+T*+G*G*A*C*A*A*C*C*T*T*C*C*A*+C* | 95005 | 18 |
| | CTT | | +T*+T | | |
| 3815 | TTTGGACAACCTTCC | A573813H | +T*+T*+T*G*G*A*C*A*A*C*C*T*T*C*C*A*+ | 95005 | 19 |
| | ACTT | | C*+T*+T | | |
| 3816 | TTTTGGACAACCTTC | A573814H | +T*+T*+T*T*G*G*A*C*A*A*C*C*T*T*C*C*+ | 95006 | 19 |
| | CACT | | A*+C*+T | | |
| 3817 | GTTTTGGACAACCTT | A573815H | +G*+T*+T*T*T*G*G*A*C*A*A*C*C*T*T*C*+ | 95007 | 19 |
| | CCAC | | C*+A*+C | | |
| 3818 | TGTTTTGGACAACCT | A573816H | +T*+G*+T*T*T*T*G*G*A*C*A*A*C*C*T*T*+ | 95008 | 19 |
| | TCCA | | C*+C*+A | | |
| 3819 | TGTTTTGGACAACCT | A573817H | +T*+G*+T*T*T*T*G*G*A*C*A*A*C*C*T*+T* | 95009 | 18 |
| | TCC | | +C*+C | | |
| 3820 | GTGTTTTGGACAACC | A573818H | +G*+T*+G*T*T*T*T*G*G*A*C*A*A*C*C*T*+ | 95009 | 19 |
| | TTCC | | T*+C*+C | | |
| 3821 | AGGGTGTTTTGGACA | A573819H | +A*+G*+G*G*T*G*T*T*T*T*G*G*A*C*A*A*+ | 95012 | 19 |
| | ACCT | | C*+C*+T | | |
| 3822 | AGGGTGTTTTGGACA | A573820H | +A*+G*+G*G*T*G*T*T*T*T*G*G*A*C*A*+A* | 95013 | 18 |
| | ACC | | +C*+C | | |
| 3823 | TAGGGTGTTTTGGAC | A573821H | +T*+A*+G*G*G*T*G*T*T*T*T*G*G*A*C*A*+ | 95013 | 19 |
| | AACC | | A*+C*+C | | |
| 3824 | CTTAGGGTGTTTTGG | A573822H | +C*+T*+T*A*G*G*G*T*G*T*T*T*T*G*G*A*+ | 95015 | 19 |
| | ACAA | | C*+A*+A | | |
| 3825 | CCTTAGGGTGTTTTG | A573823H | +C*+C*+T*T*A*G*G*G*T*G*T*T*T*T*G*G*+ | 95016 | 19 |
| | GACA | | A*+C*+A | | |
| 3826 | CCCTTAGGGTGTTTT | A573824H | +C*+C*+C*T*T*A*G*G*G*T*G*T*T*T*T*G*+ | 95017 | 19 |
| | GGAC | | G*+A*+C | | |
| 3827 | AATTTTTCCACATCTC | A573825H | +A*+A*+T*T*T*T*T*C*C*A*C*A*T*C*T*C*+ | 95047 | 19 |
| | TAC | | T*+A*+C | | |
| 3828 | GTGACATGTAGTTCT | A573826H | +G*+T*+G*A*C*A*T*G*T*A*G*T*T*C*T*T*+ | 95088 | 19 |
| | TTGG | | T*+G*+G | | |
| 3829 | ATACTGTGACATGTA | A573827H | +A*+T*+A*C*T*G*T*G*A*C*A*T*G*T*A*G*+ | 95093 | 19 |
| | GTTC | | T*+T*+C | | |
| 3830 | CTTAAAATACTGTGA | A573828H | +C*+T*+T*A*A*A*A*T*A*C*T*G*T*G*A*C*+ | 95099 | 19 |
| | CATG | | A*+T*+G | | |
| 3831 | GAGCTTAAAATACTG | A573829H | +G*+A*+G*C*T*T*A*A*A*A*T*A*C*T*G*T*+ | 95102 | 19 |
| | TGAC | | G*+A*+C | | |
| 3832 | CTAGGAGCTTAAAAT | A573830H | +C*+T*+A*G*G*A*G*C*T*T*A*A*A*A*T*A*+ | 95106 | 19 |
| | ACTG | | C*+T*+G | | |
| 3833 | TCTAGGAGCTTAAAA | A573831H | +T*+C*+T*A*G*G*A*G*C*T*T*A*A*A*A*T*+ | 95107 | 19 |
| | TACT | | A*+C*+T | | |
| 3834 | AATTTTTCTAGGAGC | A573832H | +A*+A*+T*T*T*T*T*C*T*A*G*G*A*G*C*T*+ | 95113 | 19 |
| | TTAA | | T*+A*+A | | |
| 3835 | CTGGAATTTTTCTAG | A573833H | +C*+T*+G*G*A*A*T*T*T*T*T*C*T*A*G*G*+ | 95117 | 19 |
| | GAGC | | A*+G*+C | | |
| 3836 | TCTGGAATTTTTCTA | A573834H | +T*+C*+T*G*G*A*A*T*T*T*T*T*C*T*A*G*+ | 95118 | 19 |
| | GGAG | | G*+A*+G | | |
| 3837 | ATCTGGAATTTTTCTA | A573835H | +A*+T*+C*T*G*G*A*A*T*T*T*T*T*C*T*A*+ | 95119 | 19 |
| | GGA | | G*+G*+A | | |
| 3838 | AATCTGGAATTTTTCT | A573836H | +A*+A*+T*C*T*G*G*A*A*T*T*T*T*T*C*T*+ | 95120 | 19 |
| | AGG | | A*+G*+G | | |
| 3839 | CAATCTGGAATTTTT | A573837H | +C*+A*+A*T*C*T*G*G*A*A*T*T*T*T*T*C*+ | 95121 | 19 |
| | CTAG | | T*+A*+G | | |
| 3840 | TGGCAAAGCAATCTG | A573838H | +T*+G*+G*C*A*A*A*G*C*A*A*T*C*T*G*G*+ | 95129 | 19 |
| | GAAT | | A*+A*+T | | |
| 3841 | TATTGGCAAAGCAAT | A573839H | +T*+A*+T*T*G*G*C*A*A*A*G*C*A*A*T*C*+ | 95132 | 19 |
| | CTGG | | T*+G*+G | | |
| 3842 | CTATTGGCAAAGCAA | A573840H | +C*+T*+A*T*T*G*G*C*A*A*A*G*C*A*A*T*+ | 95133 | 19 |
| | TCTG | | C*+T*+G | | |
| 3843 | CCTATTGGCAAAGCA | A573841H | +C*+C*+T*A*T*T*G*G*C*A*A*A*G*C*A*A*+ | 95134 | 19 |
| | ATCT | | T*+C*+T | | |
| 3844 | TCCTATTGGCAAAGC | A573842H | +T*+C*+C*T*A*T*T*G*G*C*A*A*A*G*C*A*+ | 95135 | 19 |
| | AATC | | A*+T*+C | | |
| 3845 | CTCCTATTGGCAAAG | A573843H | +C*+T*+C*C*T*A*T*T*G*G*C*A*A*A*G*C*+ | 95136 | 19 |
| | CAAT | | A*+A*+T | | |
| 3846 | TTCTCCTATTGGCAA | A573844H | +T*+T*+C*T*C*C*T*A*T*T*G*G*C*A*A*A*+ | 95138 | 19 |
| | AGCA | | G*+C*+A | | |
| 3847 | TCTTCTCCTATTGGC | A573845H | +T*+C*+T*T*C*T*C*C*T*A*T*T*G*G*C*A*+ | 95140 | 19 |
| | AAAG | | A*+A*+G | | |
| 3848 | ATTCTTCTCCTATTGG | A573846H | +A*+T*+T*C*T*T*C*T*C*C*T*A*T*T*G*G*+ | 95142 | 19 |
| | CAA | | C*+A*+A | | |
| 3849 | ATATTCTTCTCCTATT | A573847H | +A*+T*+A*T*T*C*T*T*C*T*C*C*T*A*T*T*+ | 95144 | 19 |
| | GGC | | G*+G*+C | | |
| 3850 | TGGAACCAGCAAATA | A573848H | +T*+G*+G*A*A*C*C*A*G*C*A*A*A*T*A*T*+ | 95156 | 19 |
| | TTCT | | T*+C*+T | | |
| 3851 | TTGGAACCAGCAAAT | A573849H | +T*+T*+G*G*A*A*C*C*A*G*C*A*A*A*T*A*+ | 95157 | 19 |
| | ATTC | | T*+T*+C | | |
| 3852 | CTTGGAACCAGCAAA | A573850H | +C*+T*+T*G*G*A*A*C*C*A*G*C*A*A*A*T*+ | 95158 | 19 |
| | TATT | | A*+T*+T | | |
| 3853 | CTGCTTGGAACCAGC | A573851H | +C*+T*+G*C*T*T*G*G*A*A*C*C*A*G*C*A*+ | 95161 | 19 |
| | AAAT | | A*+A*+T | | |
| 3854 | ATAGTGAATTTCCAT | A573852Hi | +A*+T*+A*G*T*G*A*A*T*T*T*C*C*A*T*G*+ | 95207 | 19 |
| | GTAG | | T*+A*+G | | |
| 3855 | CTATTATAGACTACA | A573853Hi | +C*+T*+A*T*T*A*T*A*G*A*C*T*A*C*A*G*+ | 95247 | 19 |
| | GTTA | | T*+T*+A | | |
| 3856 | CTATTATAGACTACA | A573854Hi | +C*+T*+A*T*T*A*T*A*G*A*C*T*A*C*A*+G* | 95248 | 18 |
| | GTT | | +T*+T | | |
| 3857 | AGTGTCAGATGCAAT | A573855Hi | +A*+G*+T*G*T*C*A*G*A*T*G*C*A*A*T*G*+ | 95340 | 19 |
| | GTTT | | T*+T*+T | | |
| 3858 | CTCTCAGTGTTGTTT | A573856Hi | +C*+T*+C*T*C*A*G*T*G*T*T*G*T*T*T*A*+ | 95489 | 19 |
| | AAGA | | A*+G*+A | | |
| 3859 | CTCTCTCAGTGTTGT | A573857Hi | +C*+T*+C*T*C*T*C*A*G*T*G*T*T*G*T*T*+ | 95491 | 19 |
| | TTAA | | T*+A*+A | | |
| 3860 | ACTCTCTCAGTGTTG | A573858Hi | +A*+C*+T*C*T*C*T*C*A*G*T*G*T*T*G*T*+ | 95492 | 19 |
| | TTTA | | T*+T*+A | | |
| 3861 | ATTTGCACATTTCAA | A573859Hi | +A*+T*+T*T*G*C*A*C*A*T*T*T*C*A*A*C*+ | 95657 | 19 |
| | CATC | | A*+T*+C | | |
| 3862 | ATTGGCTGCATCCAT | A573860Hi | +A*+T*+T*G*G*C*T*G*C*A*T*C*C*A*T*C*+ | 95683 | 19 |
| | CAAT | | A*+A*+T | | |
| 3863 | AGATGTAGATGCATG | A573861Hi | +A*+G*+A*T*G*T*A*G*A*T*G*C*A*T*G*A*+ | 95827 | 19 |
| | AAAT | | A*+A*+T | | |
| 3864 | TCAGATGTAGATGCA | A573862Hi | +T*+C*+A*G*A*T*G*T*A*G*A*T*G*C*A*T*+ | 95829 | 19 |
| | TGAA | | G*+A*+A | | |
| 3865 | CTCAGATGTAGATGC | A573863Hi | +C*+T*+C*A*G*A*T*G*T*A*G*A*T*G*C*A*+ | 95830 | 19 |
| | ATGA | | T*+G*+A | | |
| 3866 | CTCAGATGTAGATGC | A573864Hi | +C*+T*+C*A*G*A*T*G*T*A*G*A*T*G*C*+A* | 95831 | 18 |
| | ATG | | +T*+G | | |
| 3867 | TGCTCAGATGTAGAT | A573865Hi | +T*+G*+C*T*C*A*G*A*T*G*T*A*G*A*T*G*+ | 95832 | 19 |
| | GCAT | | C*+A*+T | | |
| 3868 | GCTCAGATGTAGATG | A573866Hi | +G*+C*+T*C*A*G*A*T*G*T*A*G*A*T*G*+C* | 95832 | 18 |
| | CAT | | +A*+T | | |
| 3869 | TGCTCAGATGTAGAT | A573867Hi | +T*+G*+C*T*C*A*G*A*T*G*T*A*G*A*T*+G* | 95833 | 18 |
| | GCA | | +C*+A | | |
| 3870 | GCATGCTCAGATGTA | A573868Hi | +G*+C*+A*T*G*C*T*C*A*G*A*T*G*T*A*G*+ | 95835 | 19 |
| | GATG | | A*+T*+G | | |
| 3871 | CATGCTCAGATGTAG | A573869Hi | +C*+A*+T*G*C*T*C*A*G*A*T*G*T*A*G*+A* | 95835 | 18 |
| | ATG | | +T*+G | | |
| 3872 | TGCATGCTCAGATGT | A573870Hi | +T*+G*+C*A*T*G*C*T*C*A*G*A*T*G*T*A*+ | 95836 | 19 |
| | AGAT | | G*+A*+T | | |
| 3873 | GCATGCTCAGATGTA | A573871Hi | +G*+C*+A*T*G*C*T*C*A*G*A*T*G*T*A*+G* | 95836 | 18 |
| | GAT | | +A*+T | | |
| 3874 | TCAAATGCATGCTCA | A573872Hi | +T*+C*+A*A*A*T*G*C*A*T*G*C*T*C*A*G*+ | 95841 | 19 |
| | GATG | | A*+T*+G | | |
| 3875 | TGGTTATACAGCCTC | A573873Hi | +T*+G*+G*T*T*A*T*A*C*A*G*C*C*T*C*+A* | 96000 | 18 |
| | ACA | | +C*+A | | |
| 3876 | TATGGTTATACAGCC | A573874Hi | +T*+A*+T*G*G*T*T*A*T*A*C*A*G*C*C*T*+ | 96001 | 19 |
| | TCAC | | C*+A*+C | | |
| 3877 | ACTATGGTTATACAG | A573875Hi | +A*+C*+T*A*T*G*G*T*T*A*T*A*C*A*G*+C* | 96004 | 18 |
| | CCT | | +C*+T | | |
| 3878 | ACTATGGTTATACAG | A573876Hi | +A*+C*+T*A*T*G*G*T*T*A*T*A*C*A*+G*+C | 96005 | 17 |
| | CC | | *+C | | |
| 3879 | AGGACACTATGGTTA | A573877Hi | +A*+G*+G*A*C*A*C*T*A*T*G*G*T*T*A*+T* | 96009 | 18 |
| | TAC | | +A*+C | | |
| 3880 | CTATCACAGGCCTGT | A573878H | +C*+T*+A*T*C*A*C*A*G*G*C*C*T*G*T*G*+ | 96045 | 19 |
| | GGTC | | G*+T*+C | | |
| 3881 | GGAAGCTCTATCACA | A573879H | +G*+G*+A*A*G*C*T*C*T*A*T*C*A*C*A*G*+ | 96052 | 19 |
| | GGCC | | G*+C*+C | | |
| 3882 | GGGAAGCTCTATCAC | A573880H | +G*+G*+G*A*A*G*C*T*C*T*A*T*C*A*C*A*+ | 96053 | 19 |
| | AGGC | | G*+G*+C | | |
| 3883 | TGGGGAAGCTCTATC | A573881H | +T*+G*+G*G*G*A*A*G*C*T*C*T*A*T*C*A*+ | 96055 | 19 |
| | ACAG | | C*+A*+G | | |
| 3884 | AATGGGGAAGCTCTA | A573882H | +A*+A*+T*G*G*G*G*A*A*G*C*T*C*T*A*T*+ | 96057 | 19 |
| | TCAC | | C*+A*+C | | |
| 3885 | TCACAATGGGGAAGC | A573883H | +T*+C*+A*C*A*A*T*G*G*G*G*A*A*G*C*T*+ | 96061 | 19 |
| | TCTA | | C*+T*+A | | |
| 3886 | TCTCACAATGGGGAA | A573884H | +T*+C*+T*C*A*C*A*A*T*G*G*G*G*A*A*G*+ | 96063 | 19 |
| | GCTC | | C*+T*+C | | |
| 3887 | TCTCACAATGGGGAA | A573885H | +T*+C*+T*C*A*C*A*A*T*G*G*G*G*A*A*+G* | 96064 | 18 |
| | GCT | | +C*+T | | |
| 3888 | TTCTCACAATGGGGA | A573886H | +T*+T*+C*T*C*A*C*A*A*T*G*G*G*G*A*A*+ | 96064 | 19 |
| | AGCT | | G*+C*+T | | |
| 3889 | CAGAGTTCTCACAAT | A573887H | +C*+A*+G*A*G*T*T*C*T*C*A*C*A*A*T*G*+ | 96069 | 19 |
| | GGGG | | G*+G*+G | | |
| 3890 | GATAATTTCAGAGTT | A573888H | +G*+A*+T*A*A*T*T*T*C*A*G*A*G*T*T*C*+ | 96077 | 19 |
| | CTCA | | T*+C*+A | | |
| 3891 | TGATAATTTCAGAGT | A573889H | +T*+G*+A*T*A*A*T*T*T*C*A*G*A*G*T*T*+ | 96078 | 19 |
| | TCTC | | C*+T*+C | | |
| 3892 | ATGATAATTTCAGAG | A573890H | +A*+T*+G*A*T*A*A*T*T*T*C*A*G*A*G*T*+ | 96079 | 19 |
| | TTCT | | T*+C*+T | | |
| 3893 | CGGATGATAATTTCA | A573891H | +C*+G*+G*A*T*G*A*T*A*A*T*T*T*C*A*G*+ | 96082 | 19 |
| | GAGT | | A*+G*+T | | |
| 3894 | CGGATGATAATTTCA | A573892H | +C*+G*+G*A*T*G*A*T*A*A*T*T*T*C*A*+G* | 96083 | 18 |
| | GAG | | +A*+G | | |
| 3895 | TCGGATGATAATTTC | A573893H | +T*+C*+G*G*A*T*G*A*T*A*A*T*T*T*C*A*+ | 96083 | 19 |
| | AGAG | | G*+A*+G | | |
| 3896 | TCGGATGATAATTTC | A573894H | +T*+C*+G*G*A*T*G*A*T*A*A*T*T*T*C*+A* | 96084 | 18 |
| | AGA | | +G*+A | | |
| 3897 | GTCGGATGATAATTT | A573895H | +G*+T*+C*G*G*A*T*G*A*T*A*A*T*T*T*C*+ | 96084 | 19 |
| | CAGA | | A*+G*+A | | |
| 3898 | GTCGGATGATAATTT | A573896H | +G*+T*+C*G*G*A*T*G*A*T*A*A*T*T*T*+C* | 96085 | 18 |
| | CAG | | +A*+G | | |
| 3899 | AGTCGGATGATAATT | A573897H | +A*+G*+T*C*G*G*A*T*G*A*T*A*A*T*T*T*+ | 96085 | 19 |
| | TCAG | | C*+A*+G | | |
| 3900 | TAGTCGGATGATAAT | A573898H | +T*+A*+G*T*C*G*G*A*T*G*A*T*A*A*T*T*+ | 96086 | 19 |
| | TTCA | | T*+C*+A | | |
| 3901 | TAGTCGGATGATAAT | A573899H | +T*+A*+G*T*C*G*G*A*T*G*A*T*A*A*T*+T* | 96087 | 18 |
| | TTC | | +T*+C | | |
| 3902 | ATAGTCGGATGATAA | A573900H | +A*+T*+A*G*T*C*G*G*A*T*G*A*T*A*A*T*+ | 96087 | 19 |
| | TTTC | | T*+T*+C | | |
| 3903 | TATAGTCGGATGATA | A573901H | +T*+A*+T*A*G*T*C*G*G*A*T*G*A*T*A*A*+ | 96088 | 19 |
| | ATTT | | T*+T*+T | | |
| 3904 | CATATAGTCGGATGA | A573902H | +C*+A*+T*A*T*A*G*T*C*G*G*A*T*G*A*T*+ | 96090 | 19 |
| | TAAT | | A*+A*+T | | |
| 3905 | TCATATAGTCGGATG | A573903H | +T*+C*+A*T*A*T*A*G*T*C*G*G*A*T*G*A*+ | 96091 | 19 |
| | ATAA | | T*+A*+A | | |
| 3906 | TCATATAGTCGGATG | A573904H | +T*+C*+A*T*A*T*A*G*T*C*G*G*A*T*G*+A* | 96092 | 18 |
| | ATA | | +T*+A | | |
| 3907 | TTCATATAGTCGGAT | A573905H | +T*+T*+C*A*T*A*T*A*G*T*C*G*G*A*T*G*+ | 96092 | 19 |
| | GATA | | A*+T*+A | | |
| 3908 | TTTCATATAGTCGGA | A573906H | +T*+T*+T*C*A*T*A*T*A*G*T*C*G*G*A*T*+ | 96093 | 19 |
| | TGAT | | G*+A*+T | | |
| 3909 | TTTCATATAGTCGGA | A573907H | +T*+T*+T*C*A*T*A*T*A*G*T*C*G*G*A*+T* | 96094 | 18 |
| | TGA | | +G*+A | | |
| 3910 | ATTTCATATAGTCGG | A573908H | +A*+T*+T*T*C*A*T*A*T*A*G*T*C*G*G*A*+ | 96094 | 19 |
| | ATGA | | T*+G*+A | | |
| 3911 | TTTCATATAGTCGGA | A573909H | +T*+T*+T*C*A*T*A*T*A*G*T*C*G*G*+A*+T | 96095 | 17 |
| | TG | | *+G | | |
| 3912 | ATTTCATATAGTCGG | A573910H | +A*+T*+T*T*C*A*T*A*T*A*G*T*C*G*G*+A* | 96095 | 18 |
| | ATG | | +T*+G | | |
| 3913 | CATTTCATATAGTCG | A573911H | +C*+A*+T*T*T*C*A*T*A*T*A*G*T*C*G*G*+ | 96095 | 19 |
| | GATG | | A*+T*+G | | |
| 3914 | ATTTCATATAGTCGG | A573912H | +A*+T*+T*T*C*A*T*A*T*A*G*T*C*G*+G*+A | 96096 | 17 |
| | AT | | *+T | | |
| 3915 | CATTTCATATAGTCG | A573913H | +C*+A*+T*T*T*C*A*T*A*T*A*G*T*C*G*+G* | 96096 | 18 |
| | GAT | | +A*+T | | |
| 3916 | GCATTTCATATAGTC | A573914H | +G*+C*+A*T*T*T*C*A*T*A*T*A*G*T*C*G*+ | 96096 | 19 |
| | GGAT | | G*+A*+T | | |
| 3917 | GGCATTTCATATAGT | A573915H | +G*+G*+C*A*T*T*T*C*A*T*A*T*A*G*T*C*+ | 96097 | 19 |
| | CGGA | | G*+G*+A | | |
| 3918 | GGCATTTCATATAGT | A573916H | +G*+G*+C*A*T*T*T*C*A*T*A*T*A*G*T*+C* | 96098 | 18 |
| | CGG | | +G*+G | | |
| 3919 | AGGCATTTCATATAG | A573917H | +A*+G*+G*C*A*T*T*T*C*A*T*A*T*A*G*T*+ | 96098 | 19 |
| | TCGG | | C*+G*+G | | |
| 3920 | GGCATTTCATATAGT | A573918H | +G*+G*+C*A*T*T*T*C*A*T*A*T*A*G*+T*+C | 96099 | 17 |
| | CG | | *+G | | |
| 3921 | AGGCATTTCATATAG | A573919H | +A*+G*+G*C*A*T*T*T*C*A*T*A*T*A*G*+T* | 96099 | 18 |
| | TCG | | +C*+G | | |
| 3922 | AAGGCATTTCATATA | A573920H | +A*+A*+G*G*C*A*T*T*T*C*A*T*A*T*A*G*+ | 96099 | 19 |
| | GTCG | | T*+C*+G | | |
| 3923 | AAGTAATCGATTGAT | A573921H | +A*+A*+G*T*A*A*T*C*G*A*T*T*G*A*T*+T* | 96144 | 18 |
| | TAA | | +A*+A | | |
| 3924 | CAAGTAATCGATTGA | A573922H | +C*+A*+A*G*T*A*A*T*C*G*A*T*T*G*A*T*+ | 96144 | 19 |
| | TTAA | | T*+A*+A | | |
| 3925 | CAAGTAATCGATTGA | A573923H | +C*+A*+A*G*T*A*A*T*C*G*A*T*T*G*A*+T* | 96145 | 18 |
| | TTA | | +T*+A | | |
| 3926 | TCAAGTAATCGATTG | A573924H | +T*+C*+A*A*G*T*A*A*T*C*G*A*T*T*G*A*+ | 96145 | 19 |
| | ATTA | | T*+T*+A | | |
| 3927 | TCAAGTAATCGATTG | A573925H | +T*+C*+A*A*G*T*A*A*T*C*G*A*T*T*G*+A* | 96146 | 18 |
| | ATT | | +T*+T | | |
| 3928 | CTCAAGTAATCGATT | A573926H | +C*+T*+C*A*A*G*T*A*A*T*C*G*A*T*T*G*+ | 96146 | 19 |
| | GATT | | A*+T*+T | | |
| 3929 | TCAAGTAATCGATTG | A573927H | +T*+C*+A*A*G*T*A*A*T*C*G*A*T*T*+G*+A | 96147 | 17 |
| | AT | | *+T | | |
| 3930 | CTCAAGTAATCGATT | A573928H | +C*+T*+C*A*A*G*T*A*A*T*C*G*A*T*T*+G* | 96147 | 18 |
| | GAT | | +A*+T | | |
| 3931 | TCTCAAGTAATCGAT | A573929H | +T*+C*+T*C*A*A*G*T*A*A*T*C*G*A*T*T*+ | 96147 | 19 |
| | TGAT | | G*+A*+T | | |
| 3932 | CTCAAGTAATCGATT | A573930H | +C*+T*+C*A*A*G*T*A*A*T*C*G*A*T*+T*+G | 96148 | 17 |
| | GA | | *+A | | |
| 3933 | TCTCAAGTAATCGAT | A573931H | +T*+C*+T*C*A*A*G*T*A*A*T*C*G*A*T*+T* | 96148 | 18 |
| | TGA | | +G*+A | | |
| 3934 | ATCTCAAGTAATCGA | A573932H | +A*+T*+C*T*C*A*A*G*T*A*A*T*C*G*A*T*+ | 96148 | 19 |
| | TTGA | | T*+G*+A | | |
| 3935 | AATCTCAAGTAATCG | A573933H | +A*+A*+T*C*T*C*A*A*G*T*A*A*T*C*G*A*+ | 96149 | 19 |
| | ATTG | | T*+T*+G | | |
| 3936 | AATCTCAAGTAATCG | A573934H | +A*+A*+T*C*T*C*A*A*G*T*A*A*T*C*G*+A* | 96150 | 18 |
| | ATT | | +T*+T | | |
| 3937 | AAATCTCAAGTAATC | A573935H | +A*+A*+A*T*C*T*C*A*A*G*T*A*A*T*C*G*+ | 96150 | 19 |
| | GATT | | A*+T*+T | | |
| 3938 | GAAATCTCAAGTAAT | A573936H | +G*+A*+A*A*T*C*T*C*A*A*G*T*A*A*T*C*+ | 96151 | 19 |
| | CGAT | | G*+A*+T | | |
| 3939 | GAAATCTCAAGTAAT | A573937H | +G*+A*+A*A*T*C*T*C*A*A*G*T*A*A*T*+C* | 96152 | 18 |
| | CGA | | +G*+A | | |
| 3940 | TGAAATCTCAAGTAA | A573938H | +T*+G*+A*A*A*T*C*T*C*A*A*G*T*A*A*T*+ | 96152 | 19 |
| | TCGA | | C*+G*+A | | |
| 3941 | GAAATCTCAAGTAAT | A573939H | +G*+A*+A*A*T*C*T*C*A*A*G*T*A*A*+T*+C | 96153 | 17 |
| | CG | | *+G | | |
| 3942 | TGAAATCTCAAGTAA | A573940H | +T*+G*+A*A*A*T*C*T*C*A*A*G*T*A*A*+T* | 96153 | 18 |
| | TCG | | +C*+G | | |
| 3943 | GTGAAATCTCAAGTA | A573941H | +G*+T*+G*A*A*A*T*C*T*C*A*A*G*T*A*A*+ | 96153 | 19 |
| | ATCG | | T*+C*+G | | |
| 3944 | AAGGTGAAATCTCAA | A573942H | +A*+A*+G*G*T*G*A*A*A*T*C*T*C*A*A*G*+ | 96156 | 19 |
| | GTAA | | T*+A*+A | | |
| 3945 | TAAGGTGAAATCTCA | A573943H | +T*+A*+A*G*G*T*G*A*A*A*T*C*T*C*A*A*+ | 96157 | 19 |
| | AGTA | | G*+T*+A | | |
| 3946 | TAAGGTGAAATCTCA | A573944H | +T*+A*+A*G*G*T*G*A*A*A*T*C*T*C*A*+A* | 96158 | 18 |
| | AGT | | +G*+T | | |
| 3947 | GTAAGGTGAAATCTC | A573945H | +G*+T*+A*A*G*G*T*G*A*A*A*T*C*T*C*A*+ | 96158 | 19 |
| | AAGT | | A*+G*+T | | |
| 3948 | TGTAAGGTGAAATCT | A573946H | +T*+G*+T*A*A*G*G*T*G*A*A*A*T*C*T*C*+ | 96159 | 19 |
| | CAAG | | A*+A*+G | | |
| 3949 | GCATGTAAGGTGAAA | A573947H | +G*+C*+A*T*G*T*A*A*G*G*T*G*A*A*A*T*+ | 96162 | 19 |
| | TCTC | | C*+T*+C | | |
| 3950 | AAGCATGTAAGGTGA | A573948H | +A*+A*+G*C*A*T*G*T*A*A*G*G*T*G*A*A*+ | 96164 | 19 |
| | AATC | | A*+T*+C | | |
| 3951 | AAAGCATGTAAGGTG | A573949H | +A*+A*+A*G*C*A*T*G*T*A*A*G*G*T*G*A*+ | 96165 | 19 |
| | AAAT | | A*+A*+T | | |
| 3952 | GAAAGCATGTAAGGT | A573950H | +G*+A*+A*A*G*C*A*T*G*T*A*A*G*G*T*G*+ | 96166 | 19 |
| | GAAA | | A*+A*+A | | |
| 3953 | CCTGAAAGCATGTAA | A573951H | +C*+C*+T*G*A*A*A*G*C*A*T*G*T*A*A*G*+ | 96169 | 19 |
| | GGTG | | G*+T*+G | | |
| 3954 | CCCTGAAAGCATGTA | A573952H | +C*+C*+C*T*G*A*A*A*G*C*A*T*G*T*A*A*+ | 96170 | 19 |
| | AGGT | | G*+G*+T | | |
| 3955 | CCCTGAAAGCATGTA | A573953H | +C*+C*+C*T*G*A*A*A*G*C*A*T*G*T*A*+A* | 96171 | 18 |
| | AGG | | +G*+G | | |
| 3956 | TCCCTGAAAGCATGT | A573954H | +T*+C*+C*C*T*G*A*A*A*G*C*A*T*G*T*A*+ | 96171 | 19 |
| | AAGG | | A*+G*+G | | |
| 3957 | TCTCCCTGAAAGCAT | A573955H | +T*+C*+T*C*C*C*T*G*A*A*A*G*C*A*T*G*+ | 96173 | 19 |
| | GTAA | | T*+A*+A | | |
| 3958 | ATTCTTCAGCTGCCT | A573956Hi | +A*+T*+T*C*T*T*C*A*G*C*T*G*C*C*T*T*+ | 96317 | 19 |
| | TCCA | | C*+C*+A | | |
| 3959 | TCTAATTCTTCAGCT | A573957Hi | +T*+C*+T*A*A*T*T*C*T*T*C*A*G*C*T*G*+ | 96321 | 19 |
| | GCCT | | C*+C*+T | | |
| 3960 | TGAGGTTATATGTGC | A573958Hi | +T*+G*+A*G*G*T*T*A*T*A*T*G*T*G*C*+T* | 96383 | 18 |
| | TTG | | +T*+G | | |
| 3961 | GAGGTTATATGTGCT | A573959Hi | +G*+A*+G*G*T*T*A*T*A*T*G*T*G*C*+T*+T | 96383 | 17 |
| | TG | | *+G | | |
| 3962 | AGTGGCTGTGCAACA | A573960Hi | +A*+G*+T*G*G*C*T*G*T*G*C*A*A*C*A*G*+ | 96483 | 19 |
| | GAAA | | A*+A*+A | | |
| 3963 | AAACACCGGATTTGC | A573961Hi | +A*+A*+A*C*A*C*C*G*G*A*T*T*T*G*C*T*+ | 96518 | 19 |
| | TCTT | | C*+T*+T | | |
| 3964 | AAACACCGGATTTGC | A573962Hi | +A*+A*+A*C*A*C*C*G*G*A*T*T*T*G*C*+T* | 96519 | 18 |
| | TCT | | +C*+T | | |
| 3965 | ACACCTGTGTAAAGC | A573963Hi | +A*+C*+A*C*C*T*G*T*G*T*A*A*A*G*C*T*+ | 96615 | 19 |
| | TAAT | | A*+A*+T | | |
| 3966 | TATGTTGATTAATTCC | A573964Hi | +T*+A*+T*G*T*T*G*A*T*T*A*A*T*T*C*C*+ | 96663 | 19 |
| | AAT | | A*+A*+T | | |
| 3967 | ACCTATATGTTGATT | A573965Hi | +A*+C*+C*T*A*T*A*T*G*T*T*G*A*T*T*A*+ | 96668 | 19 |
| | AATT | | A*+T*+T | | |
| 3968 | AACTACCTATATGTT | A573966Hi | +A*+A*+C*T*A*C*C*T*A*T*A*T*G*T*T*G*+ | 96672 | 19 |
| | GATT | | A*+T*+T | | |
| 3969 | ATGCTGTTAAGCAGT | A573967Hi | +A*+T*+G*C*T*G*T*T*A*A*G*C*A*G*T*T*+ | 96800 | 19 |
| | TTAA | | T*+A*+A | | |
| 3970 | GATGCTGTTAAGCAG | A573968Hi | +G*+A*+T*G*C*T*G*T*T*A*A*G*C*A*G*T*+ | 96801 | 19 |
| | TTTA | | T*+T*+A | | |
| 3971 | GTTGATGCTGTTAAG | A573969Hi | +G*+T*+T*G*A*T*G*C*T*G*T*T*A*A*G*+C* | 96805 | 18 |
| | CAG | | +A*+G | | |
| 3972 | AGGTTGATGCTGTTA | A573970Hi | +A*+G*+G*T*T*G*A*T*G*C*T*G*T*T*A*A*+ | 96806 | 19 |
| | AGCA | | G*+C*+A | | |
| 3973 | CAGGTTGATGCTGTT | A573971Hi | +C*+A*+G*G*T*T*G*A*T*G*C*T*G*T*T*A*+ | 96807 | 19 |
| | AAGC | | A*+G*+C | | |
| 3974 | AGGTTGATGCTGTTA | A573972Hi | +A*+G*+G*T*T*G*A*T*G*C*T*G*T*T*A*+A* | 96807 | 18 |
| | AGC | | +G*+C | | |
| 3975 | TCAGGTTGATGCTGT | A573973Hi | +T*+C*+A*G*G*T*T*G*A*T*G*C*T*G*T*T*+ | 96808 | 19 |
| | TAAG | | A*+A*+G | | |
| 3976 | TCCATTTCAGGTTGA | A573974Hi | +T*+C*+C*A*T*T*T*C*A*G*G*T*T*G*A*T*+ | 96814 | 19 |
| | TGCT | | G*+C*+T | | |
| 3977 | ATCCATTTCAGGTTG | A573975Hi | +A*+T*+C*C*A*T*T*T*C*A*G*G*T*T*G*A*+ | 96815 | 19 |
| | ATGC | | T*+G*+C | | |
| 3978 | TCCATTTCAGGTTGA | A573976Hi | +T*+C*+C*A*T*T*T*C*A*G*G*T*T*G*A*+T* | 96815 | 18 |
| | TGC | | +G*+C | | |
| 3979 | AAGATCCATTTCAGG | A573977Hi | +A*+A*+G*A*T*C*C*A*T*T*T*C*A*G*G*T*+ | 96818 | 19 |
| | TTGA | | T*+G*+A | | |
| 3980 | CTCATCCATATCTATT | A573978Hi | +C*+T*+C*A*T*C*C*A*T*A*T*C*T*A*T*T*+ | 96886 | 19 |
| | AAA | | A*+A*+A | | |
| 3981 | GTCCTCATCCATATC | A573979Hi | +G*+T*+C*C*T*C*A*T*C*C*A*T*A*T*C*T*+ | 96889 | 19 |
| | TATT | | A*+T*+T | | |
| 3982 | TATGAGTCCTCATCC | A573980Hi | +T*+A*+T*G*A*G*T*C*C*T*C*A*T*C*C*A*+ | 96894 | 19 |
| | ATAT | | T*+A*+T | | |
| 3983 | TATGAGTCCTCATCC | A573981Hi | +T*+A*+T*G*A*G*T*C*C*T*C*A*T*C*C*+A* | 96895 | 18 |
| | ATA | | +T*+A | | |
| 3984 | ATATGAGTCCTCATC | A573982Hi | +A*+T*+A*T*G*A*G*T*C*C*T*C*A*T*C*C*+ | 96895 | 19 |
| | CATA | | A*+T*+A | | |
| 3985 | TGGGCGAAGTGCTCG | A573983H | +T*+G*+G*G*C*G*A*A*G*T*G*C*T*C*+G*+ | 97039 | 17 |
| | TT | | T*+T | | |
| 3986 | TTGGGCGAAGTGCTC | A573984H | +T*+T*+G*G*G*C*G*A*A*G*T*G*C*T*C*+G* | 97039 | 18 |
| | GTT | | +T*+T | | |
| 3987 | TTTGGGCGAAGTGCT | A573985H | +T*+T*+T*G*G*G*C*G*A*A*G*T*G*C*T*C*+ | 97039 | 19 |
| | CGTT | | G*+T*+T | | |
| 3988 | TTGGGCGAAGTGCTC | A573986H | +T*+T*+G*G*G*C*G*A*A*G*T*G*C*T*+C*+ | 97040 | 17 |
| | GT | | G*+T | | |
| 3989 | TTTGGGCGAAGTGCT | A573987H | +T*+T*+T*G*G*G*C*G*A*A*G*T*G*C*T*+C* | 97040 | 18 |
| | CGT | | +G*+T | | |
| 3990 | GTTTGGGCGAAGTGC | A573988H | +G*+T*+T*T*G*G*G*C*G*A*A*G*T*G*C*T*+ | 97040 | 19 |
| | TCGT | | C*+G*+T | | |
| 3991 | TTTGGGCGAAGTGCT | A573989H | +T*+T*+T*G*G*G*C*G*A*A*G*T*G*C*+T*+C | 97041 | 17 |
| | CG | | *+G | | |
| 3992 | GTTTGGGCGAAGTGC | A573990H | +G*+T*+T*T*G*G*G*C*G*A*A*G*T*G*C*+T* | 97041 | 18 |
| | TCG | | +C*+G | | |
| 3993 | TGTTTGGGCGAAGTG | A573991H | +T*+G*+T*T*T*G*G*G*C*G*A*A*G*T*G*C*+ | 97041 | 19 |
| | CTCG | | T*+C*+G | | |
| 3994 | TGTTTGGGCGAAGTG | A573992H | +T*+G*+T*T*T*G*G*G*C*G*A*A*G*T*G*+C* | 97042 | 18 |
| | CTC | | +T*+C | | |
| 3995 | CTGTTTGGGCGAAGT | A573993H | +C*+T*+G*T*T*T*G*G*G*C*G*A*A*G*T*G*+ | 97042 | 19 |
| | GCTC | | C*+T*+C | | |
| 3996 | TGTTTGGGCGAAGTG | A573994H | +T*+G*+T*T*T*G*G*G*C*G*A*A*G*T*+G*+ | 97043 | 17 |
| | CT | | C*+T | | |
| 3997 | CTGTTTGGGCGAAGT | A573995H | +C*+T*+G*T*T*T*G*G*G*C*G*A*A*G*T*+G* | 97043 | 18 |
| | GCT | | +C*+T | | |
| 3998 | TCTGTTTGGGCGAAG | A573996H | +T*+C*+T*G*T*T*T*G*G*G*C*G*A*A*G*T*+ | 97043 | 19 |
| | TGCT | | G*+C*+T | | |
| 3999 | TCTGTTTGGGCGAAG | A573997H | +T*+C*+T*G*T*T*T*G*G*G*C*G*A*A*G*+T* | 97044 | 18 |
| | TGC | | +G*+C | | |
| 4000 | TTCTGTTTGGGCGAA | A573998H | +T*+T*+C*T*G*T*T*T*G*G*G*C*G*A*A*G*+ | 97044 | 19 |
| | GTGC | | T*+G*+C | | |
| 4001 | TTCTGTTTGGGCGAA | A573999H | +T*+T*+C*T*G*T*T*T*G*G*G*C*G*A*A*+G* | 97045 | 18 |
| | GTG | | +T*+G | | |
| 4002 | ATTCTGTTTGGGCGA | A574000H | +A*+T*+T*C*T*G*T*T*T*G*G*G*C*G*A*A*+ | 97045 | 19 |
| | AGTG | | G*+T*+G | | |
| 4003 | CATTCTGTTTGGGCG | A574001H | +C*+A*+T*T*C*T*G*T*T*T*G*G*G*C*G*A*+ | 97046 | 19 |
| | AAGT | | A*+G*+T | | |
| 4004 | ACATTCTGTTTGGGC | A574002H | +A*+C*+A*T*T*C*T*G*T*T*T*G*G*G*C*G*+ | 97047 | 19 |
| | GAAG | | A*+A*+G | | |
| 4005 | CAATACATTCTGTTT | A574003H | +C*+A*+A*T*A*C*A*T*T*C*T*G*T*T*T*G*+ | 97051 | 19 |
| | GGGC | | G*+G*+C | | |
| 4006 | CGCCAATACATTCTG | A574004H | +C*+G*+C*C*A*A*T*A*C*A*T*T*C*T*G*T*+ | 97054 | 19 |
| | TTTG | | T*+T*+G | | |
| 4007 | CGCCAATACATTCTG | A574005H | +C*+G*+C*C*A*A*T*A*C*A*T*T*C*T*G*+T* | 97055 | 18 |
| | TTT | | +T*+T | | |
| 4008 | TCGCCAATACATTCT | A574006H | +T*+C*+G*C*C*A*A*T*A*C*A*T*T*C*T*G*+ | 97055 | 19 |
| | GTTT | | T*+T*+T | | |
| 4009 | TCGCCAATACATTCT | A574007H | +T*+C*+G*C*C*A*A*T*A*C*A*T*T*C*T*+G* | 97056 | 18 |
| | GTT | | +T*+T | | |
| 4010 | GTCGCCAATACATTC | A574008H | +G*+T*+C*G*C*C*A*A*T*A*C*A*T*T*C*T*+ | 97056 | 19 |
| | TGTT | | G*+T*+T | | |
| 4011 | TCGCCAATACATTCT | A574009H | +T*+C*+G*C*C*A*A*T*A*C*A*T*T*C*+T*+G | 97057 | 17 |
| | GT | | *+T | | |
| 4012 | GTCGCCAATACATTC | A574010H | +G*+T*+C*G*C*C*A*A*T*A*C*A*T*T*C*+T* | 97057 | 18 |
| | TGT | | +G*+T | | |
| 4013 | TGTCGCCAATACATT | A574011H | +T*+G*+T*C*G*C*C*A*A*T*A*C*A*T*T*C*+ | 97057 | 19 |
| | CTGT | | T*+G*+T | | |
| 4014 | GTCGCCAATACATTC | A574012H | +G*+T*+C*G*C*C*A*A*T*A*C*A*T*T*+C*+T | 97058 | 17 |
| | TG | | *+G | | |
| 4015 | TGTCGCCAATACATT | A574013H | +T*+G*+T*C*G*C*C*A*A*T*A*C*A*T*T*+C* | 97058 | 18 |
| | CTG | | +T*+G | | |
| 4016 | TTGTCGCCAATACAT | A574014H | +T*+T*+G*T*C*G*C*C*A*A*T*A*C*A*T*T*+ | 97058 | 19 |
| | TCTG | | C*+T*+G | | |
| 4017 | TGTCGCCAATACATT | A574015H | +T*+G*+T*C*G*C*C*A*A*T*A*C*A*T*+T*+C | 97059 | 17 |
| | CT | | *+T | | |
| 4018 | TTGTCGCCAATACAT | A574016H | +T*+T*+G*T*C*G*C*C*A*A*T*A*C*A*T*+T* | 97059 | 18 |
| | TCT | | +C*+T | | |
| 4019 | CTTGTCGCCAATACA | A574017H | +C*+T*+T*G*T*C*G*C*C*A*A*T*A*C*A*T*+ | 97059 | 19 |
| | TTCT | | T*+C*+T | | |
| 4020 | TTGTCGCCAATACAT | A574018H | +T*+T*+G*T*C*G*C*C*A*A*T*A*C*A*+T*+T | 97060 | 17 |
| | TC | | *+C | | |
| 4021 | CTTGTCGCCAATACA | A574019H | +C*+T*+T*G*T*C*G*C*C*A*A*T*A*C*A*+T* | 97060 | 18 |
| | TTC | | +T*+C | | |
| 4022 | CCTTGTCGCCAATAC | A574020H | +C*+C*+T*T*G*T*C*G*C*C*A*A*T*A*C*A*+ | 97060 | 19 |
| | ATTC | | T*+T*+C | | |
| 4023 | CTTGTCGCCAATACA | A574021H | +C*+T*+T*G*T*C*G*C*C*A*A*T*A*C*+A*+T | 97061 | 17 |
| | TT | | *+T | | |
| 4024 | CCTTGTCGCCAATAC | A574022H | +C*+C*+T*T*G*T*C*G*C*C*A*A*T*A*C*+A* | 97061 | 18 |
| | ATT | | +T*+T | | |
| 4025 | GCCTTGTCGCCAATA | A574023H | +G*+C*+C*T*T*G*T*C*G*C*C*A*A*T*A*C*+ | 97061 | 19 |
| | CATT | | A*+T*+T | | |
| 4026 | CCTTGTCGCCAATAC | A574024H | +C*+C*+T*T*G*T*C*G*C*C*A*A*T*A*+C*+A | 97062 | 17 |
| | AT | | *+T | | |
| 4027 | GCCTTGTCGCCAATA | A574025H | +G*+C*+C*T*T*G*T*C*G*C*C*A*A*T*A*+C* | 97062 | 18 |
| | CAT | | +A*+T | | |
| 4028 | TGCCTTGTCGCCAAT | A574026H | +T*+G*+C*C*T*T*G*T*C*G*C*C*A*A*T*A*+ | 97062 | 19 |
| | ACAT | | C*+A*+T | | |
| 4029 | TCAGGAGACCAATTT | A574027H | +T*+C*+A*G*G*A*G*A*C*C*A*A*T*T*T*A*+ | 97084 | 19 |
| | AAGT | | A*+G*+T | | |
| 4030 | CTTCAGGAGACCAAT | A574028H | +C*+T*+T*C*A*G*G*A*G*A*C*C*A*A*T*T*+ | 97086 | 19 |
| | TTAA | | T*+A*+A | | |
| 4031 | GCTTCAGGAGACCAA | A574029H | +G*+C*+T*T*C*A*G*G*A*G*A*C*C*A*A*T*+ | 97087 | 19 |
| | TTTA | | T*+T*+A | | |
| 4032 | GCTTCAGGAGACCAA | A574030H | +G*+C*+T*T*C*A*G*G*A*G*A*C*C*A*A*+T* | 97088 | 18 |
| | TTT | | +T*+T | | |
| 4033 | AGCTTCAGGAGACCA | A574031H | +A*+G*+C*T*T*C*A*G*G*A*G*A*C*C*A*A*+ | 97088 | 19 |
| | ATTT | | T*+T*+T | | |
| 4034 | GCTTCAGGAGACCAA | A574032H | +G*+C*+T*T*C*A*G*G*A*G*A*C*C*A*+A*+T | 97089 | 17 |
| | TT | | *+T | | |
| 4035 | AGCTTCAGGAGACCA | A574033H | +A*+G*+C*T*T*C*A*G*G*A*G*A*C*C*A*+A* | 97089 | 18 |
| | ATT | | +T*+T | | |
| 4036 | AAGCTTCAGGAGACC | A574034H | +A*+A*+G*C*T*T*C*A*G*G*A*G*A*C*C*A*+ | 97089 | 19 |
| | AATT | | A*+T*+T | | |
| 4037 | GACAATAAGCTTCAG | A574035H | +G*+A*+C*A*A*T*A*A*G*C*T*T*C*A*G*G*+ | 97095 | 19 |
| | GAGA | | A*+G*+A | | |
| 4038 | GACAATAAGCTTCAG | A574036H | +G*+A*+C*A*A*T*A*A*G*C*T*T*C*A*G*+G* | 97096 | 18 |
| | GAG | | +A*+G | | |
| 4039 | AGACAATAAGCTTCA | A574037H | +A*+G*+A*C*A*A*T*A*A*G*C*T*T*C*A*G*+ | 97096 | 19 |
| | GGAG | | G*+A*+G | | |
| 4040 | CAGACAATAAGCTTC | A574038H | +C*+A*+G*A*C*A*A*T*A*A*G*C*T*T*C*A*+ | 97097 | 19 |
| | AGGA | | G*+G*+A | | |
| 4041 | CAGACAATAAGCTTC | A574039H | +C*+A*+G*A*C*A*A*T*A*A*G*C*T*T*C*+A* | 97098 | 18 |
| | AGG | | +G*+G | | |
| 4042 | CCAGACAATAAGCTT | A574040H | +C*+C*+A*G*A*C*A*A*T*A*A*G*C*T*T*C*+ | 97098 | 19 |
| | CAGG | | A*+G*+G | | |
| 4043 | CCAGACAATAAGCTT | A574041H | +C*+C*+A*G*A*C*A*A*T*A*A*G*C*T*T*+C* | 97099 | 18 |
| | CAG | | +A*+G | | |
| 4044 | ACCAGACAATAAGCT | A574042H | +A*+C*+C*A*G*A*C*A*A*T*A*A*G*C*T*T*+ | 97099 | 19 |
| | TCAG | | C*+A*+G | | |
| 4045 | ATCCTACCAGACAAT | A574043H | +A*+T*+C*C*T*A*C*C*A*G*A*C*A*A*T*A*+ | 97104 | 19 |
| | AAGC | | A*+G*+C | | |
| 4046 | CAGATCCTACCAGAC | A574044H | +C*+A*+G*A*T*C*C*T*A*C*C*A*G*A*C*A*+ | 97107 | 19 |
| | AATA | | A*+T*+A | | |
| 4047 | GACTTCAGATCCTAC | A574045H | +G*+A*+C*T*T*C*A*G*A*T*C*C*T*A*C*C*+ | 97112 | 19 |
| | CAGA | | A*+G*+A | | |
| 4048 | AAGACTTCAGATCCT | A574046H | +A*+A*+G*A*C*T*T*C*A*G*A*T*C*C*T*A*+ | 97114 | 19 |
| | ACCA | | C*+C*+A | | |
| 4049 | CTAAGACTTCAGATC | A574047H | +C*+T*+A*A*G*A*C*T*T*C*A*G*A*T*C*C*+ | 97116 | 19 |
| | CTAC | | T*+A*+C | | |
| 4050 | GTCTAAGACTTCAGA | A574048H | +G*+T*+C*T*A*A*G*A*C*T*T*C*A*G*A*T*+ | 97118 | 19 |
| | TCCT | | C*+C*+T | | |
| 4051 | TGTCTAAGACTTCAG | A574049H | +T*+G*+T*C*T*A*A*G*A*C*T*T*C*A*G*A*+ | 97119 | 19 |
| | ATCC | | T*+C*+C | | |
| 4052 | TGTCTAAGACTTCAG | A574050H | +T*+G*+T*C*T*A*A*G*A*C*T*T*C*A*G*+A* | 97120 | 18 |
| | ATC | | +T*+C | | |
| 4053 | TTGTCTAAGACTTCA | A574051H | +T*+T*+G*T*C*T*A*A*G*A*C*T*T*C*A*G*+ | 97120 | 19 |
| | GATC | | A*+T*+C | | |
| 4054 | TGATTGTCTAAGACT | A574052H | +T*+G*+A*T*T*G*T*C*T*A*A*G*A*C*T*T*+ | 97123 | 19 |
| | TCAG | | C*+A*+G | | |
| 4055 | TACAAGAAGGAACTG | A574053H | +T*+A*+C*A*A*G*A*A*G*G*A*A*C*T*G*T*+ | 97161 | 19 |
| | TAAT | | A*+A*+T | | |
| 4056 | TCTACAAGAAGGAAC | A574054H | +T*+C*+T*A*C*A*A*G*A*A*G*G*A*A*C*T*+ | 97163 | 19 |
| | TGTA | | G*+T*+A | | |
| 4057 | TTGGCCCAAAAGAAT | A574055H | +T*+T*+G*G*C*C*C*A*A*A*A*G*A*A*T*A*+ | 97324 | 19 |
| | ACAG | | C*+A*+G | | |
| 4058 | CTTGGCCCAAAAGAA | A574056H | +C*+T*+T*G*G*C*C*C*A*A*A*A*G*A*A*T*+ | 97325 | 19 |
| | TACA | | A*+C*+A | | |
| 4059 | CTTGGCCCAAAAGAA | A574057H | +C*+T*+T*G*G*C*C*C*A*A*A*A*G*A*A*+T* | 97326 | 18 |
| | TAC | | +A*+C | | |
| 4060 | ACTTGGCCCAAAAGA | A574058H | +A*+C*+T*T*G*G*C*C*C*A*A*A*A*G*A*A*+ | 97326 | 19 |
| | ATAC | | T*+A*+C | | |
| 4061 | AACTTGGCCCAAAAG | A574059H | +A*+A*+C*T*T*G*G*C*C*C*A*A*A*A*G*A*+ | 97327 | 19 |
| | AATA | | A*+T*+A | | |
| 4062 | CAACTTGGCCCAAAA | A574060H | +C*+A*+A*C*T*T*G*G*C*C*C*A*A*A*A*G*+ | 97328 | 19 |
| | GAAT | | A*+A*+T | | |
| 4063 | ACAACTTGGCCCAAA | A574061H | +A*+C*+A*A*C*T*T*G*G*C*C*C*A*A*A*A*+ | 97329 | 19 |
| | AGAA | | G*+A*+A | | |
| 4064 | GTCCACAACTTGGCC | A574062H | +G*+T*+C*C*A*C*A*A*C*T*T*G*G*C*C*C*+ | 97333 | 19 |
| | CAAA | | A*+A*+A | | |
| 4065 | GTGGTCCACAACTTG | A574063H | +G*+T*+G*G*T*C*C*A*C*A*A*C*T*T*G*G*+ | 97336 | 19 |
| | GCCC | | C*+C*+C | | |
| 4066 | CAATGTGGTCCACAA | A574064H | +C*+A*+A*T*G*T*G*G*T*C*C*A*C*A*A*C*+ | 97340 | 19 |
| | CTTG | | T*+T*+G | | |
| 4067 | ATCAATGTGGTCCAC | A574065H | +A*+T*+C*A*A*T*G*T*G*G*T*C*C*A*C*A*+ | 97342 | 19 |
| | AACT | | A*+C*+T | | |
| 4068 | ATCAATGTGGTCCAC | A574066H | +A*+T*+C*A*A*T*G*T*G*G*T*C*C*A*C*+A* | 97343 | 18 |
| | AAC | | +A*+C | | |
| 4069 | AATCAATGTGGTCCA | A574067H | +A*+A*+T*C*A*A*T*G*T*G*G*T*C*C*A*C*+ | 97343 | 19 |
| | CAAC | | A*+A*+C | | |
| 4070 | AGAGAATCAATGTGG | A574068H | +A*+G*+A*G*A*A*T*C*A*A*T*G*T*G*G*T*+ | 97347 | 19 |
| | TCCA | | C*+C*+A | | |
| 4071 | TGAGAGAATCAATGT | A574069H | +T*+G*+A*G*A*G*A*A*T*C*A*A*T*G*T*G*+ | 97349 | 19 |
| | GGTC | | G*+T*+C | | |
| 4072 | TCCAGCAACCCAGGA | A574070H | +T*+C*+C*A*G*C*A*A*C*C*C*A*G*G*A*A*+ | 97377 | 19 |
| | AACC | | A*+C*+C | | |
| 4073 | TCCAGCAACCCAGGA | A574071H | +T*+C*+C*A*G*C*A*A*C*C*C*A*G*G*A*+A* | 97378 | 18 |
| | AAC | | +A*+C | | |
| 4074 | CTCCAGCAACCCAGG | A574072H | +C*+T*+C*C*A*G*C*A*A*C*C*C*A*G*G*A*+ | 97378 | 19 |
| | AAAC | | A*+A*+C | | |
| 4075 | ATCTCCAGCAACCCA | A574073H | +A*+T*+C*T*C*C*A*G*C*A*A*C*C*C*A*G*+ | 97380 | 19 |
| | GGAA | | G*+A*+A | | |
| 4076 | ATCAATCTCCAGCAA | A574074H | +A*+T*+C*A*A*T*C*T*C*C*A*G*C*A*A*C*+ | 97384 | 19 |
| | CCCA | | C*+C*+A | | |
| 4077 | TATCAATCTCCAGCA | A574075H | +T*+A*+T*C*A*A*T*C*T*C*C*A*G*C*A*A*+ | 97385 | 19 |
| | ACCC | | C*+C*+C | | |
| 4078 | TATCAATCTCCAGCA | A574076H | +T*+A*+T*C*A*A*T*C*T*C*C*A*G*C*A*+A* | 97386 | 18 |
| | ACC | | +C*+C | | |
| 4079 | ATATCAATCTCCAGC | A574077H | +A*+T*+A*T*C*A*A*T*C*T*C*C*A*G*C*A*+ | 97386 | 19 |
| | AACC | | A*+C*+C | | |
| 4080 | TCACCACAAATATCA | A574078H | +T*+C*+A*C*C*A*C*A*A*A*T*A*T*C*A*A*+ | 97395 | 19 |
| | ATCT | | T*+C*+T | | |
| 4081 | TCACCACAAATATCA | A574079H | +T*+C*+A*C*C*A*C*A*A*A*T*A*T*C*A*+A* | 97396 | 18 |
| | ATC | | +T*+C | | |
| 4082 | TTCACCACAAATATC | A574080H | +T*+T*+C*A*C*C*A*C*A*A*A*T*A*T*C*A*+ | 97396 | 19 |
| | AATC | | A*+T*+C | | |
| 4083 | CTCCTTCACCACAAA | A574081H | +C*+T*+C*C*T*T*C*A*C*C*A*C*A*A*A*T*+ | 97400 | 19 |
| | TATC | | A*+T*+C | | |
| 4084 | TTCTCCTTCACCACA | A574082H | +T*+T*+C*T*C*C*T*T*C*A*C*C*A*C*A*A*+ | 97402 | 19 |
| | AATA | | A*+T*+A | | |
| 4085 | TTCTCCTTCACCACA | A574083H | +T*+T*+C*T*C*C*T*T*C*A*C*C*A*C*A*+A* | 97403 | 18 |
| | AAT | | +A*+T | | |
| 4086 | TTTCTCCTTCACCACA | A574084H | +T*+T*+T*C*T*C*C*T*T*C*A*C*C*A*C*A*+ | 97403 | 19 |
| | AAT | | A*+A*+T | | |
| 4087 | ACAGAGTTTCTCCTT | A574085H | +A*+C*+A*G*A*G*T*T*T*C*T*C*C*T*T*C*+ | 97409 | 19 |
| | CACC | | A*+C*+C | | |
| 4088 | AACAGAGTTTCTCCT | A574086H | +A*+A*+C*A*G*A*G*T*T*T*C*T*C*C*T*T*+ | 97410 | 19 |
| | TCAC | | C*+A*+C | | |
| 4089 | CAACAGAGTTTCTCC | A574087H | +C*+A*+A*C*A*G*A*G*T*T*T*C*T*C*C*T*+ | 97411 | 19 |
| | TTCA | | T*+C*+A | | |
| 4090 | TCAACAGAGTTTCTC | A574088H | +T*+C*+A*A*C*A*G*A*G*T*T*T*C*T*C*C*+ | 97412 | 19 |
| | CTTC | | T*+T*+C | | |
| 4091 | CCATTTCTTCAACAG | A574089H | +C*+C*+A*T*T*T*C*T*T*C*A*A*C*A*G*A*+ | 97420 | 19 |
| | AGTT | | G*+T*+T | | |
| 4092 | TAATGCCCATTTCTTC | A574090H | +T*+A*+A*T*G*C*C*C*A*T*T*T*C*T*T*C*+ | 97426 | 19 |
| | AAC | | A*+A*+C | | |
| 4093 | ATATAATGCCCATTT | A574091H | +A*+T*+A*T*A*A*T*G*C*C*C*A*T*T*T*C*+ | 97429 | 19 |
| | CTTC | | T*+T*+C | | |
| 4094 | CTTCACCATCATTAA | A574092H | +C*+T*+T*C*A*C*C*A*T*C*A*T*T*A*A*A*+ | 97448 | 19 |
| | AACT | | A*+C*+T | | |
| 4095 | ATCAAGTAAGATTTT | A574093H | +A*+T*+C*A*A*G*T*A*A*G*A*T*T*T*T*T*+ | 97471 | 19 |
| | TTGA | | T*+G*+A | | |
| 4096 | CATCAAGTAAGATTT | A574094H | +C*+A*+T*C*A*A*G*T*A*A*G*A*T*T*T*T*+ | 97472 | 19 |
| | TTTG | | T*+T*+G | | |
| 4097 | TCAAGTCATCAAGTA | A574095H | +T*+C*+A*A*G*T*C*A*T*C*A*A*G*T*A*A*+ | 97478 | 19 |
| | AGAT | | G*+A*+T | | |
| 4098 | ATCAAGTCATCAAGT | A574096H | +A*+T*+C*A*A*G*T*C*A*T*C*A*A*G*T*A*+ | 97479 | 19 |
| | AAGA | | A*+G*+A | | |
| 4099 | ATCAAGTCATCAAGT | A574097H | +A*+T*+C*A*A*G*T*C*A*T*C*A*A*G*T*+A* | 97480 | 18 |
| | AAG | | +A*+G | | |
| 4100 | CATCAAGTCATCAAG | A574098H | +C*+A*+T*C*A*A*G*T*C*A*T*C*A*A*G*T*+ | 97480 | 19 |
| | TAAG | | A*+A*+G | | |
| 4101 | TCATCAAGTCATCAA | A574099H | +T*+C*+A*T*C*A*A*G*T*C*A*T*C*A*A*G*+ | 97481 | 19 |
| | GTAA | | T*+A*+A | | |
| 4102 | GCTTTCTTCATCAAG | A574100H | +G*+C*+T*T*T*C*T*T*C*A*T*C*A*A*G*T*+ | 97488 | 19 |
| | TCAT | | C*+A*+T | | |
| 4103 | TCTGAATCTAGAATA | A574101Hi | +T*+C*+T*G*A*A*T*C*T*A*G*A*A*T*A*C*+ | 97682 | 19 |
| | CAAG | | A*+A*+G | | |
| 4104 | GCTGTTATTATATGG | A574102Hi | +G*+C*+T*G*T*T*A*T*T*A*T*A*T*G*G*C*+ | 97842 | 19 |
| | CAGA | | A*+G*+A | | |
| 4105 | TTGCCACATTCATGA | A574103Hi | +T*+T*+G*C*C*A*C*A*T*T*C*A*T*G*A*A*+ | 97864 | 19 |
| | AGCT | | G*+C*+T | | |
| 4106 | CATCTGACTAGAAAG | A574104Hi | +C*+A*+T*C*T*G*A*C*T*A*G*A*A*A*G*T*+ | 97896 | 19 |
| | TTAT | | T*+A*+T | | |
| 4107 | GACATCTGACTAGAA | A574105Hi | +G*+A*+C*A*T*C*T*G*A*C*T*A*G*A*A*A*+ | 97898 | 19 |
| | AGTT | | G*+T*+T | | |
| 4108 | CTACCAGACATCTGA | A574106Hi | +C*+T*+A*C*C*A*G*A*C*A*T*C*T*G*A*+C* | 97905 | 18 |
| | CTA | | +T*+A | | |
| 4109 | CAGACTACCAGACAT | A574107Hi | +C*+A*+G*A*C*T*A*C*C*A*G*A*C*A*T*C*+ | 97908 | 19 |
| | CTGA | | T*+G*+A | | |
| 4110 | GCAGACTACCAGACA | A574108Hi | +G*+C*+A*G*A*C*T*A*C*C*A*G*A*C*A*T*+ | 97909 | 19 |
| | TCTG | | C*+T*+G | | |
| 4111 | ACTGCTGCAGACTAC | A574109Hi | +A*+C*+T*G*C*T*G*C*A*G*A*C*T*A*C*C*+ | 97915 | 19 |
| | CAGA | | A*+G*+A | | |
| 4112 | GAACTGCTGCAGACT | A574110Hi | +G*+A*+A*C*T*G*C*T*G*C*A*G*A*C*T*A*+ | 97917 | 19 |
| | ACCA | | C*+C*+A | | |
| 4113 | TGAACTGCTGCAGAC | A574111Hi | +T*+G*+A*A*C*T*G*C*T*G*C*A*G*A*C*T*+ | 97918 | 19 |
| | TACC | | A*+C*+C | | |
| 4114 | TCTGAACTGCTGCAG | A574112Hi | +T*+C*+T*G*A*A*C*T*G*C*T*G*C*A*G*A*+ | 97920 | 19 |
| | ACTA | | C*+T*+A | | |
| 4115 | CTGAACTGCTGCAGA | A574113Hi | +C*+T*+G*A*A*C*T*G*C*T*G*C*A*G*A*+C* | 97920 | 18 |
| | CTA | | +T*+A | | |
| 4116 | ACGTTCACTTGTAGA | A574114Hi | +A*+C*+G*T*T*C*A*C*T*T*G*T*A*G*A*+A* | 97938 | 18 |
| | ATT | | +T*+T | | |
| 4117 | CTACGTTCACTTGTA | A574115Hi | +C*+T*+A*C*G*T*T*C*A*C*T*T*G*T*A*+G* | 97940 | 18 |
| | GAA | | +A*+A | | |
| 4118 | CCTACGTTCACTTGT | A574116Hi | +C*+C*+T*A*C*G*T*T*C*A*C*T*T*G*+T*+A | 97942 | 17 |
| | AG | | *+G | | |
| 4119 | TCCTACGTTCACTTG | A574117Hi | +T*+C*+C*T*A*C*G*T*T*C*A*C*T*T*G*+T* | 97942 | 18 |
| | TAG | | +A*+G | | |
| 4120 | TATTCCTACGTTCACT | A574118Hi | +T*+A*+T*T*C*C*T*A*C*G*T*T*C*A*C*T*+ | 97944 | 19 |
| | TGT | | T*+G*+T | | |
| 4121 | TATTCCTACGTTCACT | A574119Hi | +T*+A*+T*T*C*C*T*A*C*G*T*T*C*A*C*+T* | 97945 | 18 |
| | TG | | +T*+G | | |
| 4122 | AACAACATTTAAGGA | A574120Hi | +A*+A*+C*A*A*C*A*T*T*T*A*A*G*G*A*G*+ | 98114 | 19 |
| | GATA | | A*+T*+A | | |
| 4123 | GATTTACTAAGAGAT | A574121H | +G*+A*+T*T*T*A*C*T*A*A*G*A*G*A*T*+C* | 98164 | 18 |
| | CTC | | +T*+C | | |
| 4124 | GGATTTACTAAGAGA | A574122H | +G*+G*+A*T*T*T*A*C*T*A*A*G*A*G*A*T*+ | 98164 | 19 |
| | TCTC | | C*+T*+C | | |
| 4125 | GGTTGATCTGGATTT | A574123H | +G*+G*+T*T*G*A*T*C*T*G*G*A*T*T*T*A*+ | 98173 | 19 |
| | ACTA | | C*+T*+A | | |
| 4126 | TGGTTGATCTGGATT | A574124H | +T*+G*+G*T*T*G*A*T*C*T*G*G*A*T*T*T*+ | 98174 | 19 |
| | TACT | | A*+C*+T | | |
| 4127 | TGGTTGATCTGGATT | A574125H | +T*+G*+G*T*T*G*A*T*C*T*G*G*A*T*T*+T* | 98175 | 18 |
| | TAC | | +A*+C | | |
| 4128 | TTGGTTGATCTGGAT | A574126H | +T*+T*+G*G*T*T*G*A*T*C*T*G*G*A*T*T*+ | 98175 | 19 |
| | TTAC | | T*+A*+C | | |
| 4129 | CCTTGGTTGATCTGG | A574127H | +C*+C*+T*T*G*G*T*T*G*A*T*C*T*G*G*A*+ | 98177 | 19 |
| | ATTT | | T*+T*+T | | |
| 4130 | GCCTTGGTTGATCTG | A574128H | +G*+C*+C*T*T*G*G*T*T*G*A*T*C*T*G*G*+ | 98178 | 19 |
| | GATT | | A*+T*+T | | |
| 4131 | GCCTTGGTTGATCTG | A574129H | +G*+C*+C*T*T*G*G*T*T*G*A*T*C*T*G*+G* | 98179 | 18 |
| | GAT | | +A*+T | | |
| 4132 | AGCCTTGGTTGATCT | A574130H | +A*+G*+C*C*T*T*G*G*T*T*G*A*T*C*T*G*+ | 98179 | 19 |
| | GGAT | | G*+A*+T | | |
| 4133 | GAGCCTTGGTTGATC | A574131H | +G*+A*+G*C*C*T*T*G*G*T*T*G*A*T*C*T*+ | 98180 | 19 |
| | TGGA | | G*+G*+A | | |
| 4134 | GAGCCTTGGTTGATC | A574132H | +G*+A*+G*C*C*T*T*G*G*T*T*G*A*T*C*+T* | 98181 | 18 |
| | TGG | | +G*+G | | |
| 4135 | TGAGCCTTGGTTGAT | A574133H | +T*+G*+A*G*C*C*T*T*G*G*T*T*G*A*T*C*+ | 98181 | 19 |
| | CTGG | | T*+G*+G | | |
| 4136 | GTGAGCCTTGGTTGA | A574134H | +G*+T*+G*A*G*C*C*T*T*G*G*T*T*G*A*T*+ | 98182 | 19 |
| | TCTG | | C*+T*+G | | |
| 4137 | GTGAGCCTTGGTTGA | A574135H | +G*+T*+G*A*G*C*C*T*T*G*G*T*T*G*A*+T* | 98183 | 18 |
| | TCT | | +C*+T | | |
| 4138 | GGTGAGCCTTGGTTG | A574136H | +G*+G*+T*G*A*G*C*C*T*T*G*G*T*T*G*A*+ | 98183 | 19 |
| | ATCT | | T*+C*+T | | |
| 4139 | GGTGAGCCTTGGTTG | A574137H | +G*+G*+T*G*A*G*C*C*T*T*G*G*T*T*G*+A* | 98184 | 18 |
| | ATC | | +T*+C | | |
| 4140 | TGGTGAGCCTTGGTT | A574138H | +T*+G*+G*T*G*A*G*C*C*T*T*G*G*T*T*G*+ | 98184 | 19 |
| | GATC | | A*+T*+C | | |
| 4141 | AATGGTGAGCCTTGG | A574139H | +A*+A*+T*G*G*T*G*A*G*C*C*T*T*G*G*T*+ | 98186 | 19 |
| | TTGA | | T*+G*+A | | |
| 4142 | AATGGTGAGCCTTGG | A574140H | +A*+A*+T*G*G*T*G*A*G*C*C*T*T*G*G*+T* | 98187 | 18 |
| | TTG | | +T*+G | | |
| 4143 | GAATGGTGAGCCTTG | A574141H | +G*+A*+A*T*G*G*T*G*A*G*C*C*T*T*G*G*+ | 98187 | 19 |
| | GTTG | | T*+T*+G | | |
| 4144 | GAATGGTGAGCCTTG | A574142H | +G*+A*+A*T*G*G*T*G*A*G*C*C*T*T*G*+G* | 98188 | 18 |
| | GTT | | +T*+T | | |
| 4145 | GGAATGGTGAGCCTT | A574143H | +G*+G*+A*A*T*G*G*T*G*A*G*C*C*T*T*G*+ | 98188 | 19 |
| | GGTT | | G*+T*+T | | |
| 4146 | TGGAATGGTGAGCCT | A574144H | +T*+G*+G*A*A*T*G*G*T*G*A*G*C*C*T*T*+ | 98189 | 19 |
| | TGGT | | G*+G*+T | | |
| 4147 | TATTGGAATGGTGAG | A574145H | +T*+A*+T*T*G*G*A*A*T*G*G*T*G*A*G*C*+ | 98192 | 19 |
| | CCTT | | C*+T*+T | | |
| 4148 | TATTGGAATGGTGAG | A574146H | +T*+A*+T*T*G*G*A*A*T*G*G*T*G*A*G*+C* | 98193 | 18 |
| | CCT | | +C*+T | | |
| 4149 | ATATTGGAATGGTGA | A574147H | +A*+T*+A*T*T*G*G*A*A*T*G*G*T*G*A*G*+ | 98193 | 19 |
| | GCCT | | C*+C*+T | | |
| 4150 | CAATCTGAGATATTG | A574148H | +C*+A*+A*T*C*T*G*A*G*A*T*A*T*T*G*G*+ | 98202 | 19 |
| | GAAT | | A*+A*+T | | |
| 4151 | GGCAATCTGAGATAT | A574149H | +G*+G*+C*A*A*T*C*T*G*A*G*A*T*A*T*T*+ | 98204 | 19 |
| | TGGA | | G*+G*+A | | |
| 4152 | GGCAATCTGAGATAT | A574150H | +G*+G*+C*A*A*T*C*T*G*A*G*A*T*A*T*+T* | 98205 | 18 |
| | TGG | | +G*+G | | |
| 4153 | GGGCAATCTGAGATA | A574151H | +G*+G*+G*C*A*A*T*C*T*G*A*G*A*T*A*T*+ | 98205 | 19 |
| | TTGG | | T*+G*+G | | |
| 4154 | GGGCAATCTGAGATA | A574152H | +G*+G*+G*C*A*A*T*C*T*G*A*G*A*T*A*+T* | 98206 | 18 |
| | TTG | | +T*+G | | |
| 4155 | GGGGCAATCTGAGAT | A574153H | +G*+G*+G*G*C*A*A*T*C*T*G*A*G*A*T*A*+ | 98206 | 19 |
| | ATTG | | T*+T*+G | | |
| 4156 | GGGGCAATCTGAGAT | A574154H | +G*+G*+G*G*C*A*A*T*C*T*G*A*G*A*T*+A* | 98207 | 18 |
| | ATT | | +T*+T | | |
| 4157 | AGGGGCAATCTGAGA | A574155H | +A*+G*+G*G*G*C*A*A*T*C*T*G*A*G*A*T*+ | 98207 | 19 |
| | TATT | | A*+T*+T | | |
| 4158 | AGGGGCAATCTGAGA | A574156H | +A*+G*+G*G*G*C*A*A*T*C*T*G*A*G*A*+T* | 98208 | 18 |
| | TAT | | +A*+T | | |
| 4159 | CAGGGGCAATCTGAG | A574157H | +C*+A*+G*G*G*G*C*A*A*T*C*T*G*A*G*A* | 98208 | 19 |
| | ATAT | | +T*+A*+T | | |
| 4160 | GTCAGGGGCAATCTG | A574158H | +G*+T*+C*A*G*G*G*G*C*A*A*T*C*T*G*A*+ | 98210 | 19 |
| | AGAT | | G*+A*+T | | |
| 4161 | CAAGTCAGGGGCAAT | A574159H | +C*+A*+A*G*T*C*A*G*G*G*G*C*A*A*T*C*+ | 98213 | 19 |
| | CTGA | | T*+G*+A | | |
| 4162 | TCAAGTCAGGGGCAA | A574160H | +T*+C*+A*A*G*T*C*A*G*G*G*G*C*A*A*T*+ | 98214 | 19 |
| | TCTG | | C*+T*+G | | |
| 4163 | ATCAAGTCAGGGGCA | A574161H | +A*+T*+C*A*A*G*T*C*A*G*G*G*G*C*A*A*+ | 98215 | 19 |
| | ATCT | | T*+C*+T | | |
| 4164 | ATCAAGTCAGGGGCA | A574162H | +A*+T*+C*A*A*G*T*C*A*G*G*G*G*C*A*+A* | 98216 | 18 |
| | ATC | | +T*+C | | |
| 4165 | AATCAAGTCAGGGGC | A574163H | +A*+A*+T*C*A*A*G*T*C*A*G*G*G*G*C*A*+ | 98216 | 19 |
| | AATC | | A*+T*+C | | |
| 4166 | AATCAAGTCAGGGGC | A574164H | +A*+A*+T*C*A*A*G*T*C*A*G*G*G*G*C*+A* | 98217 | 18 |
| | AAT | | +A*+T | | |
| 4167 | AAATCAAGTCAGGGG | A574165H | +A*+A*+A*T*C*A*A*G*T*C*A*G*G*G*G*C*+ | 98217 | 19 |
| | CAAT | | A*+A*+T | | |
| 4168 | CAAAATCAAGTCAGG | A574166H | +C*+A*+A*A*A*T*C*A*A*G*T*C*A*G*G*G*+ | 98219 | 19 |
| | GGCA | | G*+C*+A | | |
| 4169 | CCAAAATCAAGTCAG | A574167H | +C*+C*+A*A*A*A*T*C*A*A*G*T*C*A*G*G*+ | 98220 | 19 |
| | GGGC | | G*+G*+C | | |
| 4170 | CAGCCAAAATCAAGT | A574168H | +C*+A*+G*C*C*A*A*A*A*T*C*A*A*G*T*C*+ | 98223 | 19 |
| | CAGG | | A*+G*+G | | |
| 4171 | GTCAGCCAAAATCAA | A574169H | +G*+T*+C*A*G*C*C*A*A*A*A*T*C*A*A*G*+ | 98225 | 19 |
| | GTCA | | T*+C*+A | | |
| 4172 | GGTCAGCCAAAATCA | A574170H | +G*+G*+T*C*A*G*C*C*A*A*A*A*T*C*A*A*+ | 98226 | 19 |
| | AGTC | | G*+T*+C | | |
| 4173 | GGTCAGCCAAAATCA | A574171H | +G*+G*+T*C*A*G*C*C*A*A*A*A*T*C*A*+A* | 98227 | 18 |
| | AGT | | +G*+T | | |
| 4174 | AGGTCAGCCAAAATC | A574172H | +A*+G*+G*T*C*A*G*C*C*A*A*A*A*T*C*A*+ | 98227 | 19 |
| | AAGT | | A*+G*+T | | |
| 4175 | CAGGTCAGCCAAAAT | A574173H | +C*+A*+G*G*T*C*A*G*C*C*A*A*A*A*T*C*+ | 98228 | 19 |
| | CAAG | | A*+A*+G | | |
| 4176 | GCAGGTCAGCCAAAA | A574174H | +G*+C*+A*G*G*T*C*A*G*C*C*A*A*A*A*T*+ | 98229 | 19 |
| | TCAA | | C*+A*+A | | |
| 4177 | GGCAGGTCAGCCAAA | A574175H | +G*+G*+C*A*G*G*T*C*A*G*C*C*A*A*A*A* | 98230 | 19 |
| | ATCA | | +T*+C*+A | | |
| 4178 | GGCAGGTCAGCCAAA | A574176H | +G*+G*+C*A*G*G*T*C*A*G*C*C*A*A*A*+A | 98231 | 18 |
| | ATC | | *+T*+C | | |
| 4179 | AGGCAGGTCAGCCAA | A574177H | +A*+G*+G*C*A*G*G*T*C*A*G*C*C*A*A*A* | 98231 | 19 |
| | AATC | | +A*+T*+C | | |
| 4180 | TAGGCAGGTCAGCCA | A574178H | +T*+A*+G*G*C*A*G*G*T*C*A*G*C*C*A*A*+ | 98232 | 19 |
| | AAAT | | A*+A*+T | | |
| 4181 | CTAGGCAGGTCAGCC | A574179H | +C*+T*+A*G*G*C*A*G*G*T*C*A*G*C*C*A*+ | 98233 | 19 |
| | AAAA | | A*+A*+A | | |
| 4182 | CTAGGCAGGTCAGCC | A574180H | +C*+T*+A*G*G*C*A*G*G*T*C*A*G*C*C*+A* | 98234 | 18 |
| | AAA | | +A*+A | | |
| 4183 | TCTAGGCAGGTCAGC | A574181H | +T*+C*+T*A*G*G*C*A*G*G*T*C*A*G*C*C*+ | 98234 | 19 |
| | CAAA | | A*+A*+A | | |
| 4184 | TTCTAGGCAGGTCAG | A574182H | +T*+T*+C*T*A*G*G*C*A*G*G*T*C*A*G*C*+ | 98235 | 19 |
| | CCAA | | C*+A*+A | | |
| 4185 | TTTCTAGGCAGGTCA | A574183H | +T*+T*+T*C*T*A*G*G*C*A*G*G*T*C*A*G*+ | 98236 | 19 |
| | GCCA | | C*+C*+A | | |
| 4186 | ATTTCTAGGCAGGTC | A574184H | +A*+T*+T*T*C*T*A*G*G*C*A*G*G*T*C*A*+ | 98237 | 19 |
| | AGCC | | G*+C*+C | | |
| 4187 | ATATTTCTAGGCAGG | A574185H | +A*+T*+A*T*T*T*C*T*A*G*G*C*A*G*G*T*+ | 98239 | 19 |
| | TCAG | | C*+A*+G | | |
| 4188 | ATAATATTTCTAGGC | A574186H | +A*+T*+A*A*T*A*T*T*T*C*T*A*G*G*C*A*+ | 98242 | 19 |
| | AGGT | | G*+G*+T | | |
| 4189 | CAACATAATATTTCTA | A574187H | +C*+A*+A*C*A*T*A*A*T*A*T*T*T*C*T*A*+ | 98246 | 19 |
| | GGC | | G*+G*+C | | |
| 4190 | GGAGCTTGTTCAAAT | A574188H | +G*+G*+A*G*C*T*T*G*T*T*C*A*A*A*T*T*+ | 98278 | 19 |
| | TCCA | | C*+C*+A | | |
| 4191 | ACTCTGGAGCTTGTT | A574189H | +A*+C*+T*C*T*G*G*A*G*C*T*T*G*T*T*C*+ | 98283 | 19 |
| | CAAA | | A*+A*+A | | |
| 4192 | AACTCTGGAGCTTGT | A574190H | +A*+A*+C*T*C*T*G*G*A*G*C*T*T*G*T*T*+ | 98284 | 19 |
| | TCAA | | C*+A*+A | | |
| 4193 | AAACTCTGGAGCTTG | A574191H | +A*+A*+A*C*T*C*T*G*G*A*G*C*T*T*G*T*+ | 98285 | 19 |
| | TTCA | | T*+C*+A | | |
| 4194 | AAACTCTGGAGCTTG | A574192H | +A*+A*+A*C*T*C*T*G*G*A*G*C*T*T*G*+T* | 98286 | 18 |
| | TTC | | +T*+C | | |
| 4195 | GAAACTCTGGAGCTT | A574193H | +G*+A*+A*A*C*T*C*T*G*G*A*G*C*T*T*G*+ | 98286 | 19 |
| | GTTC | | T*+T*+C | | |
| 4196 | TAGGAGAAACTCTGG | A574194H | +T*+A*+G*G*A*G*A*A*A*C*T*C*T*G*G*A*+ | 98291 | 19 |
| | AGCT | | G*+C*+T | | |
| 4197 | TAGGAGAAACTCTGG | A574195H | +T*+A*+G*G*A*G*A*A*A*C*T*C*T*G*G*+A* | 98292 | 18 |
| | AGC | | +G*+C | | |
| 4198 | CTAGGAGAAACTCTG | A574196H | +C*+T*+A*G*G*A*G*A*A*A*C*T*C*T*G*G*+ | 98292 | 19 |
| | GAGC | | A*+G*+C | | |
| 4199 | TTCTAAATTATAAGG | A574197Hi | +T*+T*+C*T*A*A*A*T*T*A*T*A*A*G*G*A*+ | 98355 | 19 |
| | AGAA | | G*+A*+A | | |
| 4200 | GATCCTAGTCTACAT | A574198Hi | +G*+A*+T*C*C*T*A*G*T*C*T*A*C*A*T*+G* | 98500 | 18 |
| | GAT | | +A*+T | | |
| 4201 | TGATCCTAGTCTACA | A574199Hi | +T*+G*+A*T*C*C*T*A*G*T*C*T*A*C*A*+T* | 98501 | 18 |
| | TGA | | +G*+A | | |
| 4202 | TTGATCCTAGTCTAC | A574200Hi | +T*+T*+G*A*T*C*C*T*A*G*T*C*T*A*C*+A* | 98502 | 18 |
| | ATG | | +T*+G | | |
| 4203 | TCCTCATCTTGATCCT | A574201Hi | +T*+C*+C*T*C*A*T*C*T*T*G*A*T*C*C*T*+ | 98509 | 19 |
| | AGT | | A*+G*+T | | |
| 4204 | TGGCAATTAAGGCAA | A574202Hi | +T*+G*+G*C*A*A*T*T*A*A*G*G*C*A*A*A*+ | 98571 | 19 |
| | ATAC | | T*+A*+C | | |
| 4205 | GGCAATTAAGGCAAA | A574203Hi | +G*+G*+C*A*A*T*T*A*A*G*G*C*A*A*A*+T* | 98571 | 18 |
| | TAC | | +A*+C | | |
| 4206 | CTGGCAATTAAGGCA | A574204Hi | +C*+T*+G*G*C*A*A*T*T*A*A*G*G*C*A*A*+ | 98572 | 19 |
| | AATA | | A*+T*+A | | |
| 4207 | ATGGCTGCTTCACTT | A574205Hi | +A*+T*+G*G*C*T*G*C*T*T*C*A*C*T*T*+A* | 98626 | 18 |
| | AAC | | +A*+C | | |
| 4208 | TGAGGTGTGCTTCCT | A574206Hi | +T*+G*+A*G*G*T*G*T*G*C*T*T*C*C*T*T*+ | 98666 | 19 |
| | TTAC | | T*+A*+C | | |
| 4209 | GAGGTGTGCTTCCTT | A574207Hi | +G*+A*+G*G*T*G*T*G*C*T*T*C*C*T*T*+T* | 98666 | 18 |
| | TAC | | +A*+C | | |
| 4210 | ACTGAGGTGTGCTTC | A574208Hi | +A*+C*+T*G*A*G*G*T*G*T*G*C*T*T*C*C*+ | 98668 | 19 |
| | CTTT | | T*+T*+T | | |
| 4211 | CACTGAGGTGTGCTT | A574209Hi | +C*+A*+C*T*G*A*G*G*T*G*T*G*C*T*T*C*+ | 98669 | 19 |
| | CCTT | | C*+T*+T | | |
| 4212 | ACTGAGGTGTGCTTC | A574210Hi | +A*+C*+T*G*A*G*G*T*G*T*G*C*T*T*C*+C* | 98669 | 18 |
| | CTT | | +T*+T | | |
| 4213 | ACACTGAGGTGTGCT | A574211Hi | +A*+C*+A*C*T*G*A*G*G*T*G*T*G*C*T*T*+ | 98670 | 19 |
| | TCCT | | C*+C*+T | | |
| 4214 | CTACACTGAGGTGTG | A574212Hi | +C*+T*+A*C*A*C*T*G*A*G*G*T*G*T*G*C*+ | 98672 | 19 |
| | CTTC | | T*+T*+C | | |
| 4215 | CTACACTGAGGTGTG | A574213Hi | +C*+T*+A*C*A*C*T*G*A*G*G*T*G*T*G*+C* | 98673 | 18 |
| | CTT | | +T*+T | | |
| 4216 | AATTCAGCATTGTCA | A574214Hi | +A*+A*+T*T*C*A*G*C*A*T*T*G*T*C*A*C*+ | 98714 | 19 |
| | CACT | | A*+C*+T | | |
| 4217 | CTATGAGAATTCAGC | A574215Hi | +C*+T*+A*T*G*A*G*A*A*T*T*C*A*G*C*A*+ | 98721 | 19 |
| | ATTG | | T*+T*+G | | |
| 4218 | ATGAATATTTCTATG | A574216Hi | +A*+T*+G*A*A*T*A*T*T*T*C*T*A*T*G*A*+ | 98731 | 19 |
| | AGAA | | G*+A*+A | | |
| 4219 | CTACATGAATTCAAT | A574217Hi | +C*+T*+A*C*A*T*G*A*A*T*T*C*A*A*T*G*+ | 98889 | 19 |
| | GATT | | A*+T*+T | | |
| 4220 | GTTACCATGACTTCA | A574218Hi | +G*+T*+T*A*C*C*A*T*G*A*C*T*T*C*A*C*+ | 98931 | 19 |
| | CCTT | | C*+T*+T | | |
| 4221 | CAGGAAGTCAAGATA | A574219Hi | +C*+A*+G*G*A*A*G*T*C*A*A*G*A*T*A*C*+ | 98968 | 19 |
| | CAAA | | A*+A*+A | | |
| 4222 | TAAATAGCTAAACTT | A574220Hi | +T*+A*+A*A*T*A*G*C*T*A*A*A*C*T*T*C*+ | 99319 | 19 |
| | CTCC | | T*+C*+C | | |
| 4223 | TTATGTCCTGACTGA | A574221Hi | +T*+T*+A*T*G*T*C*C*T*G*A*C*T*G*A*+C* | 99613 | 18 |
| | CAG | | +A*+G | | |
| 4224 | ATTATGTCCTGACTG | A574222Hi | +A*+T*+T*A*T*G*T*C*C*T*G*A*C*T*G*A*+ | 99613 | 19 |
| | ACAG | | C*+A*+G | | |
| 4225 | TTCCTGTATACCTTG | A574223Hi | +T*+T*+C*C*T*G*T*A*T*A*C*C*T*T*G*A*+ | 99833 | 19 |
| | AAGT | | A*+G*+T | | |
| 4226 | TCCTGTATACCTTGA | A574224Hi | +T*+C*+C*T*G*T*A*T*A*C*C*T*T*G*A*+A* | 99833 | 18 |
| | AGT | | +G*+T | | |
| 4227 | CTGGTACTTTCCTGT | A574225Hi | +C*+T*+G*G*T*A*C*T*T*T*C*C*T*G*T*A*+ | 99841 | 19 |
| | ATAC | | T*+A*+C | | |
| 4228 | GACGTTTCCACATAC | A574226Hi | +G*+A*+C*G*T*T*T*C*C*A*C*A*T*A*C*A*+ | 99970 | 19 |
| | ACTA | | C*+T*+A | | |
| 4229 | TGACGTTTCCACATA | A574227Hi | +T*+G*+A*C*G*T*T*T*C*C*A*C*A*T*A*C*+ | 99971 | 19 |
| | CACT | | A*+C*+T | | |
| 4230 | ATGACGTTTCCACAT | A574228Hi | +A*+T*+G*A*C*G*T*T*T*C*C*A*C*A*T*A*+ | 99972 | 19 |
| | ACAC | | C*+A*+C | | |
| 4231 | TGACGTTTCCACATA | A574229Hi | +T*+G*+A*C*G*T*T*T*C*C*A*C*A*T*A*+C* | 99972 | 18 |
| | CAC | | +A*+C | | |
| 4232 | AATGACGTTTCCACA | A574230Hi | +A*+A*+T*G*A*C*G*T*T*T*C*C*A*C*A*T*+ | 99973 | 19 |
| | TACA | | A*+C*+A | | |
| 4233 | AATGACGTTTCCACA | A574231Hi | +A*+A*+T*G*A*C*G*T*T*T*C*C*A*C*A*+T* | 99974 | 18 |
| | TAC | | +A*+C | | |
| 4234 | TTTGTAAATGACGTT | A574232Hi | +T*+T*+T*G*T*A*A*A*T*G*A*C*G*T*T*T*+ | 99979 | 19 |
| | TCCA | | C*+C*+A | | |
| 4235 | CTTTGTAAATGACGT | A574233Hi | +C*+T*+T*T*G*T*A*A*A*T*G*A*C*G*T*+T* | 99981 | 18 |
| | TTC | | +T*+C | | |
| 4236 | ACTTTGTAAATGACG | A574234Hi | +A*+C*+T*T*T*G*T*A*A*A*T*G*A*C*G*T*+ | 99981 | 19 |
| | TTTC | | T*+T*+C | | |
| 4237 | AGACTTTGTAAATGA | A574235Hi | +A*+G*+A*C*T*T*T*G*T*A*A*A*T*G*A*+C* | 99984 | 18 |
| | CGT | | +G*+T | | |
| 4238 | CAGACTTTGTAAATG | A574236Hi | +C*+A*+G*A*C*T*T*T*G*T*A*A*A*T*G*+A* | 99985 | 18 |
| | ACG | | +C*+G | | |
| 4239 | ACAGACTTTGTAAAT | A574237Hi | +A*+C*+A*G*A*C*T*T*T*G*T*A*A*A*T*G*+ | 99985 | 19 |
| | GACG | | A*+C*+G | | |
| 4240 | AACAGACTTTGTAAA | A574238Hi | +A*+A*+C*A*G*A*C*T*T*T*G*T*A*A*A*T*+ | 99986 | 19 |
| | TGAC | | G*+A*+C | | |
| 4241 | GCCAACTAAACAGAC | A574239Hi | +G*+C*+C*A*A*C*T*A*A*A*C*A*G*A*C*T*+ | 99994 | 19 |
| | TTTG | | T*+T*+G | | |
| 4242 | GGCCAACTAAACAGA | A574240Hi | +G*+G*+C*C*A*A*C*T*A*A*A*C*A*G*A*C*+ | 99995 | 19 |
| | CTTT | | T*+T*+T | | |
| 4243 | GGCCAACTAAACAGA | A574241Hi | +G*+G*+C*C*A*A*C*T*A*A*A*C*A*G*A*+C* | 99996 | 18 |
| | CTT | | +T*+T | | |
| 4244 | TCTAGACTGCTTTCT | A574242Hi | +T*+C*+T*A*G*A*C*T*G*C*T*T*T*C*T*G*+ | 100406 | 19 |
| | GTAA | | T*+A*+A | | |
| 4245 | TATCTAGACTGCTTT | A574243Hi | +T*+A*+T*C*T*A*G*A*C*T*G*C*T*T*T*C*+ | 100408 | 19 |
| | CTGT | | T*+G*+T | | |
| 4246 | ACTCCTATCTAGACT | A574244Hi | +A*+C*+T*C*C*T*A*T*C*T*A*G*A*C*T*+G* | 100414 | 18 |
| | GCT | | +C*+T | | |
| 4247 | TCTTTGGCAACATCC | A574245Hi | +T*+C*+T*T*T*G*G*C*A*A*C*A*T*C*C*+G* | 100590 | 18 |
| | GGA | | +G*+A | | |
| 4248 | ATCTTTGGCAACATC | A574246Hi | +A*+T*+C*T*T*T*G*G*C*A*A*C*A*T*+C*+C | 100592 | 17 |
| | CG | | *+G | | |
| 4249 | TATCTTTGGCAACAT | A574247Hi | +T*+A*+T*C*T*T*T*G*G*C*A*A*C*A*T*+C* | 100592 | 18 |
| | CCG | | +C*+G | | |
| 4250 | GAAGGATACACCTAA | A574248Hi | +G*+A*+A*G*G*A*T*A*C*A*C*C*T*A*A*+T* | 100641 | 18 |
| | TTT | | +T*+T | | |
| 4251 | ATATAAAGATGCTGG | A574249Hi | +A*+T*+A*T*A*A*A*G*A*T*G*C*T*G*G*A*+ | 100680 | 19 |
| | AGTC | | G*+T*+C | | |
| 4252 | TATAAAGATGCTGGA | A574250Hi | +T*+A*+T*A*A*A*G*A*T*G*C*T*G*G*A*+G* | 100680 | 18 |
| | GTC | | +T*+C | | |
| 4253 | AATATAAAGATGCTG | A574251Hi | +A*+A*+T*A*T*A*A*A*G*A*T*G*C*T*G*G*+ | 100681 | 19 |
| | GAGT | | A*+G*+T | | |
| 4254 | CTATTCATATCTATTA | A574252Hi | +C*+T*+A*T*T*C*A*T*A*T*C*T*A*T*T*A*+ | 100840 | 19 |
| | AGG | | A*+G*+G | | |
| 4255 | GTCTATTCATATCTAT | A574253Hi | +G*+T*+C*T*A*T*T*C*A*T*A*T*C*T*A*T*+ | 100842 | 19 |
| | TAA | | T*+A*+A | | |
| 4256 | AAGTCTATTCATATCT | A574254Hi | +A*+A*+G*T*C*T*A*T*T*C*A*T*A*T*C*T*+ | 100844 | 19 |
| | ATT | | A*+T*+T | | |
| 4257 | ATTGAAGGCAAGGAA | A574255Hi | +A*+T*+T*G*A*A*G*G*C*A*A*G*G*A*A*A*+ | 101017 | 19 |
| | ATTT | | T*+T*+T | | |
| 4258 | AATTGAAGGCAAGGA | A574256Hi | +A*+A*+T*T*G*A*A*G*G*C*A*A*G*G*A*A*+ | 101018 | 19 |
| | AATT | | A*+T*+T | | |
| 4259 | GATTGTCATGTCTTT | A574257Hi | +G*+A*+T*T*G*T*C*A*T*G*T*C*T*T*T*C*+ | 101103 | 19 |
| | CAGG | | A*+G*+G | | |
| 4260 | GGATTGTCATGTCTT | A574258Hi | +G*+G*+A*T*T*G*T*C*A*T*G*T*C*T*T*T*+ | 101104 | 19 |
| | TCAG | | C*+A*+G | | |
| 4261 | TGGATTGTCATGTCT | A574259Hi | +T*+G*+G*A*T*T*G*T*C*A*T*G*T*C*T*T*+ | 101105 | 19 |
| | TTCA | | T*+C*+A | | |
| 4262 | AGTATGGATTGTCAT | A574260Hi | +A*+G*+T*A*T*G*G*A*T*T*G*T*C*A*T*+G* | 101110 | 18 |
| | GTC | | +T*+C | | |
| 4263 | GGTCGGAGAGATGGA | A574261Hi | +G*+G*+T*C*G*G*A*G*A*G*A*T*G*G*A*+A | 101643 | 18 |
| | AAC | | *+A*+C | | |
| 4264 | GTCGGAGAGATGGAA | A574262Hi | +G*+T*+C*G*G*A*G*A*G*A*T*G*G*A*+A*+ | 101643 | 17 |
| | AC | | A*+C | | |
| 4265 | AGGATTGTTGGTATT | A574263Hi | +A*+G*+G*A*T*T*G*T*T*G*G*T*A*T*T*+G* | 101765 | 18 |
| | GAG | | +A*+G | | |
| 4266 | ACCATTAGTTAGGAT | A574264Hi | +A*+C*+C*A*T*T*A*G*T*T*A*G*G*A*T*+T* | 101775 | 18 |
| | TGT | | +G*+T | | |
| 4267 | TGGTATTCTCTCATTC | A574265Hi | +T*+G*+G*T*A*T*T*C*T*C*T*C*A*T*T*C*+ | 101824 | 19 |
| | TAT | | T*+A*+T | | |
| 4268 | TTGGTATTCTCTCATT | A574266Hi | +T*+T*+G*G*T*A*T*T*C*T*C*T*C*A*T*T*+ | 101825 | 19 |
| | CTA | | C*+T*+A | | |
| 4269 | ATCTTATCAGTCTTG | A574267Hi | +A*+T*+C*T*T*A*T*C*A*G*T*C*T*T*G*G*+ | 101837 | 19 |
| | GTAT | | T*+A*+T | | |
| 4270 | GCATCTTATCAGTCT | A574268Hi | +G*+C*+A*T*C*T*T*A*T*C*A*G*T*C*T*T*+ | 101839 | 19 |
| | TGGT | | G*+G*+T | | |
| 4271 | TATTTGCATCTTATCA | A574269Hi | +T*+A*+T*T*T*G*C*A*T*C*T*T*A*T*C*A*+ | 101844 | 19 |
| | GTC | | G*+T*+C | | |
| 4272 | GAAATTACACATCTA | A574270Hi | +G*+A*+A*A*T*T*A*C*A*C*A*T*C*T*A*T*+ | 102004 | 19 |
| | TGGA | | G*+G*+A | | |
| 4273 | GGTGAAATTACACAT | A574271Hi | +G*+G*+T*G*A*A*A*T*T*A*C*A*C*A*T*C*+ | 102007 | 19 |
| | CTAT | | T*+A*+T | | |
| 4274 | TGGATATAAGTGAAT | A574272Hi | +T*+G*+G*A*T*A*T*A*A*G*T*G*A*A*T*A*+ | 102064 | 19 |
| | ACAC | | C*+A*+C | | |
| 4275 | CTTGATGGATATAAG | A574273Hi | +C*+T*+T*G*A*T*G*G*A*T*A*T*A*A*G*T*+ | 102069 | 19 |
| | TGAA | | G*+A*+A | | |
| 4276 | GTCACTTGATGGATA | A574274Hi | +G*+T*+C*A*C*T*T*G*A*T*G*G*A*T*A*T*+ | 102073 | 19 |
| | TAAG | | A*+A*+G | | |
| 4277 | GAAATGTAGTCACTT | A574275Hi | +G*+A*+A*A*T*G*T*A*G*T*C*A*C*T*T*G*+ | 102081 | 19 |
| | GATG | | A*+T*+G | | |
| 4278 | TGCCTTGAAATGTAG | A574276Hi | +T*+G*+C*C*T*T*G*A*A*A*T*G*T*A*G*T*+ | 102087 | 19 |
| | TCAC | | C*+A*+C | | |
| 4279 | GTGCCTTGAAATGTA | A574277Hi | +G*+T*+G*C*C*T*T*G*A*A*A*T*G*T*A*G*+ | 102088 | 19 |
| | GTCA | | T*+C*+A | | |
| 4280 | GTACAATATTTATGG | A574278Hi | +G*+T*+A*C*A*A*T*A*T*T*T*A*T*G*G*T*+ | 102113 | 19 |
| | TTCT | | T*+C*+T | | |
| 4281 | GCAAATCCTGTTTGT | A574279Hi | +G*+C*+A*A*A*T*C*C*T*G*T*T*T*G*T*A*+ | 102126 | 19 |
| | ACAA | | C*+A*+A | | |
| 4282 | CTGAGCATGCTTTGT | A574280Hi | +C*+T*+G*A*G*C*A*T*G*C*T*T*T*G*T*A*+ | 102269 | 19 |
| | ACCT | | C*+C*+T | | |
| 4283 | CTGAGCATGCTTTGT | A574281Hi | +C*+T*+G*A*G*C*A*T*G*C*T*T*T*G*T*+A* | 102270 | 18 |
| | ACC | | +C*+C | | |
| 4284 | ATTTCCTGAGCATGC | A574282Hi | +A*+T*+T*T*C*C*T*G*A*G*C*A*T*G*C*T*+ | 102274 | 19 |
| | TTTG | | T*+T*+G | | |
| 4285 | AAGTGCTCTAGACAA | A574283Hi | +A*+A*+G*T*G*C*T*C*T*A*G*A*C*A*A*A*+ | 102304 | 19 |
| | ACCG | | C*+C*+G | | |
| 4286 | AGTGCTCTAGACAAA | A574284Hi | +A*+G*+T*G*C*T*C*T*A*G*A*C*A*A*A*+C* | 102304 | 18 |
| | CCG | | +C*+G | | |
| 4287 | ACAAGTGCTCTAGAC | A574285Hi | +A*+C*+A*A*G*T*G*C*T*C*T*A*G*A*C*A*+ | 102306 | 19 |
| | AAAC | | A*+A*+C | | |
| 4288 | AATTCAGCTCCATAG | A574286Hi | +A*+A*+T*T*C*A*G*C*T*C*C*A*T*A*G*T*+ | 102570 | 19 |
| | TCAT | | C*+A*+T | | |
| 4289 | CTAAATTCAGCTCCA | A574287Hi | +C*+T*+A*A*A*T*T*C*A*G*C*T*C*C*A*T*+ | 102573 | 19 |
| | TAGT | | A*+G*+T | | |
| 4290 | TCTAAATTCAGCTCC | A574288Hi | +T*+C*+T*A*A*A*T*T*C*A*G*C*T*C*C*A*+ | 102574 | 19 |
| | ATAG | | T*+A*+G | | |
| 4291 | TCATTCCACTTTGATT | A574289Hi | +T*+C*+A*T*T*C*C*A*C*T*T*T*G*A*T*T*+ | 102643 | 19 |
| | GTG | | G*+T*+G | | |
| 4292 | ATCATTCCACTTTGAT | A574290Hi | +A*+T*+C*A*T*T*C*C*A*C*T*T*T*G*A*T*+ | 102644 | 19 |
| | TGT | | T*+G*+T | | |
| 4293 | TCCTATCATTCCACTT | A574291Hi | +T*+C*+C*T*A*T*C*A*T*T*C*C*A*C*T*T*+ | 102648 | 19 |
| | TGA | | T*+G*+A | | |
| 4294 | GTTCCTTACTTCCAA | A574292Hi | +G*+T*+T*C*C*T*T*A*C*T*T*C*C*A*A*C*+ | 102693 | 19 |
| | CTCA | | T*+C*+A | | |
| 4295 | CTGAGTTCCTTACTT | A574293Hi | +C*+T*+G*A*G*T*T*C*C*T*T*A*C*T*T*C*+ | 102697 | 19 |
| | CCAA | | C*+A*+A | | |
| 4296 | TGAGTTCCTTACTTC | A574294Hi | +T*+G*+A*G*T*T*C*C*T*T*A*C*T*T*C*+C* | 102697 | 18 |
| | CAA | | +A*+A | | |
| 4297 | TTTCCACTACCACCA | A574295Hi | +T*+T*+T*C*C*A*C*T*A*C*C*A*C*C*A*C*+ | 102760 | 19 |
| | CACC | | A*+C*+C | | |
| 4298 | TCCATATAGCTGATG | A574296Hi | +T*+C*+C*A*T*A*T*A*G*C*T*G*A*T*G*A*+ | 102912 | 19 |
| | ATAA | | T*+A*+A | | |
| 4299 | TTCTACCTTCCATATA | A574297Hi | +T*+T*+C*T*A*C*C*T*T*C*C*A*T*A*T*A*+ | 102920 | 19 |
| | GCT | | G*+C*+T | | |
| 4300 | CAACTCAATCACAGC | A574298Hi | +C*+A*+A*C*T*C*A*A*T*C*A*C*A*G*C*C*+ | 102980 | 19 |
| | CTAT | | T*+A*+T | | |
| 4301 | CTTCCAACTCAATCA | A574299Hi | +C*+T*+T*C*C*A*A*C*T*C*A*A*T*C*A*C*+ | 102984 | 19 |
| | CAGC | | A*+G*+C | | |
| 4302 | TAGCCACCTCCTAAG | A574300Hi | +T*+A*+G*C*C*A*C*C*T*C*C*T*A*A*G*T*+ | 103047 | 19 |
| | TGCT | | G*+C*+T | | |
| 4303 | TCACTTATAGCCACC | A574301Hi | +T*+C*+A*C*T*T*A*T*A*G*C*C*A*C*C*T*+ | 103054 | 19 |
| | TCCT | | C*+C*+T | | |
| 4304 | TTCACTTATAGCCAC | A574302Hi | +T*+T*+C*A*C*T*T*A*T*A*G*C*C*A*C*C*+ | 103055 | 19 |
| | CTCC | | T*+C*+C | | |
| 4305 | TCACTTATAGCCACC | A574303Hi | +T*+C*+A*C*T*T*A*T*A*G*C*C*A*C*C*+T* | 103055 | 18 |
| | TCC | | +C*+C | | |
| 4306 | TATGTCATCCACTTCT | A574304Hi | +T*+A*+T*G*T*C*A*T*C*C*A*C*T*T*C*T*+ | 103112 | 19 |
| | GTG | | G*+T*+G | | |
| 4307 | TTATGTCATCCACTTC | A574305Hi | +T*+T*+A*T*G*T*C*A*T*C*C*A*C*T*T*C*+ | 103113 | 19 |
| | TGT | | T*+G*+T | | |
| 4308 | CCTGCTCAATTATGT | A574306Hi | +C*+C*+T*G*C*T*C*A*A*T*T*A*T*G*T*C*+ | 103122 | 19 |
| | CATC | | A*+T*+C | | |
| 4309 | ATCAAATCTCCTTTAA | A574307Hi | +A*+T*+C*A*A*A*T*C*T*C*C*T*T*T*A*A*+ | 103218 | 19 |
| | CAT | | C*+A*+T | | |
| 4310 | AATCAAATCTCCTTTA | A574308Hi | +A*+A*+T*C*A*A*A*T*C*T*C*C*T*T*T*A*+ | 103219 | 19 |
| | ACA | | A*+C*+A | | |
| 4311 | TTCATTGAATCAAAT | A574309Hi | +T*+T*+C*A*T*T*G*A*A*T*C*A*A*A*T*C*+ | 103226 | 19 |
| | CTCC | | T*+C*+C | | |
| 4312 | GTTTCATTGAATCAA | A574310Hi | +G*+T*+T*T*C*A*T*T*G*A*A*T*C*A*A*A*+ | 103228 | 19 |
| | ATCT | | T*+C*+T | | |
| 4313 | TGTTTCATTGAATCA | A574311Hi | +T*+G*+T*T*T*C*A*T*T*G*A*A*T*C*A*A*+ | 103229 | 19 |
| | AATC | | A*+T*+C | | |
| 4314 | GTTTCATTGAATCAA | A574312Hi | +G*+T*+T*T*C*A*T*T*G*A*A*T*C*A*A*+A* | 103229 | 18 |
| | ATC | | +T*+C | | |
| 4315 | AATACAAAGCACTTA | A574313Hi | +A*+A*+T*A*C*A*A*A*G*C*A*C*T*T*A*A*+ | 103332 | 19 |
| | AATT | | A*+T*+T | | |
| 4316 | AACTGATCCAAAACT | A574314H | +A*+A*+C*T*G*A*T*C*C*A*A*A*A*C*T*G*+ | 103368 | 19 |
| | GCCA | | C*+C*+A | | |
| 4317 | AAACTGATCCAAAAC | A574315H | +A*+A*+A*C*T*G*A*T*C*C*A*A*A*A*C*T*+ | 103369 | 19 |
| | TGCC | | G*+C*+C | | |
| 4318 | TCCTTCATAGGCTGC | A574316H | +T*+C*+C*T*T*C*A*T*A*G*G*C*T*G*C*+T* | 103390 | 18 |
| | TCG | | +C*+G | | |
| 4319 | CTCCTTCATAGGCTG | A574317H | +C*+T*+C*C*T*T*C*A*T*A*G*G*C*T*G*C*+ | 103390 | 19 |
| | CTCG | | T*+C*+G | | |
| 4320 | TCACAGCCACTTCTT | A574318H | +T*+C*+A*C*A*G*C*C*A*C*T*T*C*T*T*C*+ | 103405 | 19 |
| | CTCC | | T*+C*+C | | |
| 4321 | AATCTTCACAGCCAC | A574319H | +A*+A*+T*C*T*T*C*A*C*A*G*C*C*A*C*T*+ | 103410 | 19 |
| | TTCT | | T*+C*+T | | |
| 4322 | GCAGCTAGGAGTCTG | A574320Hi | +G*+C*+A*G*C*T*A*G*G*A*G*T*C*T*G*+A* | 103864 | 18 |
| | ACT | | +C*+T | | |
| 4323 | GCTAGGCTTCAAGGT | A574321Hi | +G*+C*+T*A*G*G*C*T*T*C*A*A*G*G*T*+C* | 103895 | 18 |
| | CTG | | +T*+G | | |
| 4324 | CTTGCTAGGCTTCAA | A574322Hi | +C*+T*+T*G*C*T*A*G*G*C*T*T*C*A*A*+G* | 103898 | 18 |
| | GGT | | +G*+T | | |
| 4325 | AAGTCAGGAGCTTGC | A574323Hi | +A*+A*+G*T*C*A*G*G*A*G*C*T*T*G*C*+T* | 103908 | 18 |
| | TAG | | +A*+G | | |
| 4326 | AGGTGCAAGCTTATC | A574324Hi | +A*+G*+G*T*G*C*A*A*G*C*T*T*A*T*+C*+A | 104100 | 17 |
| | AG | | *+G | | |
| 4327 | TCCATCTTTGTAGCA | A574325Hi | +T*+C*+C*A*T*C*T*T*T*G*T*A*G*C*A*+A* | 104161 | 18 |
| | AGT | | +G*+T | | |
| 4328 | GTAGGAAATAGGATA | A574326Hi | +G*+T*+A*G*G*A*A*A*T*A*G*G*A*T*A*+A* | 104191 | 18 |
| | AGC | | +G*+C | | |
| 4329 | AGTCAGTCCATGCTG | A574327Hi | +A*+G*+T*C*A*G*T*C*C*A*T*G*C*T*G*+T* | 104327 | 18 |
| | TCT | | +C*+T | | |
| 4330 | ATAGCAAGGAGTCAG | A574328Hi | +A*+T*+A*G*C*A*A*G*G*A*G*T*C*A*G*+T* | 104336 | 18 |
| | TCC | | +C*+C | | |
| 4331 | CTCATCTCACATAGC | A574329Hi | +C*+T*+C*A*T*C*T*C*A*C*A*T*A*G*C*+A* | 104346 | 18 |
| | AAG | | +A*+G | | |
| 4332 | GCAGTAAGTCATGTG | A574330Hi | +G*+C*+A*G*T*A*A*G*T*C*A*T*G*T*G*+C* | 105020 | 18 |
| | CTG | | +T*+G | | |
| 4333 | CTCTCCTGTTTAGAG | A574331Hi | +C*+T*+C*T*C*C*T*G*T*T*T*A*G*A*G*+C* | 105034 | 18 |
| | CAG | | +A*+G | | |
| 4334 | TCCATCTCTCCTGTTT | A574332Hi | +T*+C*+C*A*T*C*T*C*T*C*C*T*G*T*T*+T* | 105039 | 18 |
| | AG | | +A*+G | | |
| 4335 | ATCCATCTCTCCTGTT | A574333Hi | +A*+T*+C*C*A*T*C*T*C*T*C*C*T*G*T*+T* | 105040 | 18 |
| | TA | | +T*+A | | |
| 4336 | TAACTCATAGATTAA | A574334Hi | +T*+A*+A*C*T*C*A*T*A*G*A*T*T*A*A*+T* | 105077 | 18 |
| | TCA | | +C*+A | | |
| 4337 | CTAACTCATAGATTA | A574335Hi | +C*+T*+A*A*C*T*C*A*T*A*G*A*T*T*A*+A* | 105078 | 18 |
| | ATC | | +T*+C | | |
| 4338 | GTGGTAACTTGAACA | A574336Hi | +G*+T*+G*G*T*A*A*C*T*T*G*A*A*C*A*+A* | 106255 | 18 |
| | AGC | | +G*+C | | |
| 4339 | AGTGGTAACTTGAAC | A574337Hi | +A*+G*+T*G*G*T*A*A*C*T*T*G*A*A*C*+A* | 106256 | 18 |
| | AAG | | +A*+G | | |
| 4340 | GAAAGTGGTAACTTG | A574338Hi | +G*+A*+A*A*G*T*G*G*T*A*A*C*T*T*G*+A* | 106259 | 18 |
| | AAC | | +A*+C | | |
| 4341 | GCAAGTTATTGATCT | A574339Hi | +G*+C*+A*A*G*T*T*A*T*T*G*A*T*C*T*+A* | 106746 | 18 |
| | ATG | | +T*+G | | |
| 4342 | TGTAGGCCATTTGAT | A574340Hi | +T*+G*+T*A*G*G*C*C*A*T*T*T*G*A*T*+T* | 107017 | 18 |
| | TCC | | +C*+C | | |
| 4343 | GGTTATTATGTAGGC | A574341Hi | +G*+G*+T*T*A*T*T*A*T*G*T*A*G*G*C*+C* | 107025 | 18 |
| | CAT | | +A*+T | | |
| 4344 | ACTGTTAACTAACAG | A574342Hi | +A*+C*+T*G*T*T*A*A*C*T*A*A*C*A*G*+T* | 107313 | 18 |
| | TGT | | +G*+T | | |
| 4345 | GTTGAGTTCTGGTAT | A574343Hi | +G*+T*+T*G*A*G*T*T*C*T*G*G*T*A*T*+A* | 107464 | 18 |
| | AAG | | +A*+G | | |
| 4346 | AGTTGAGTTCTGGTA | A574344Hi | +A*+G*+T*T*G*A*G*T*T*C*T*G*G*T*A*+T* | 107465 | 18 |
| | TAA | | +A*+A | | |
| 4347 | GATCATATTTGCTTA | A574345Hi | +G*+A*+T*C*A*T*A*T*T*T*G*C*T*T*A*+G* | 108224 | 18 |
| | GTA | | +T*+A | | |
| 4348 | GACTGTGATCATATT | A574346Hi | +G*+A*+C*T*G*T*G*A*T*C*A*T*A*T*T*+T* | 108230 | 18 |
| | TGC | | +G*+C | | |
| 4349 | CAACCAGCTTCAAAG | A574347Hi | +C*+A*+A*C*C*A*G*C*T*T*C*A*A*A*G*+A* | 108360 | 18 |
| | ATG | | +T*+G | | |
| 4350 | TGCTTCTTAACAACC | A574348Hi | +T*+G*+C*T*T*C*T*T*A*A*C*A*A*C*C*+A* | 108370 | 18 |
| | AGC | | +G*+C | | |
| 4351 | CCTGGCTGCAACAAT | A574349Hi | +C*+C*+T*G*G*C*T*G*C*A*A*C*A*A*T*+C* | 108700 | 18 |
| | CTC | | +T*+C | | |
| 4352 | AATCAGCTGCAACTG | A574350Hi | +A*+A*+T*C*A*G*C*T*G*C*A*A*C*T*G*+G* | 109144 | 18 |
| | GCA | | +C*+A | | |
| 4353 | ACTAATCAGCTGCAA | A574351Hi | +A*+C*+T*A*A*T*C*A*G*C*T*G*C*A*A*+C* | 109147 | 18 |
| | CTG | | +T*+G | | |
| 4354 | ACTACTAATCAGCTG | A574352Hi | +A*+C*+T*A*C*T*A*A*T*C*A*G*C*T*G*+C* | 109150 | 18 |
| | CAA | | +A*+A | | |
| 4355 | AGCCACTACTAATCA | A574353Hi | +A*+G*+C*C*A*C*T*A*C*T*A*A*T*C*A*+G* | 109154 | 18 |
| | GCT | | +C*+T | | |
| 4356 | TATAGCCACTACTAA | A574354Hi | +T*+A*+T*A*G*C*C*A*C*T*A*C*T*A*A*+T* | 109157 | 18 |
| | TCA | | +C*+A | | |
| 4357 | TCTCTATAGCCACTA | A574355Hi | +T*+C*+T*C*T*A*T*A*G*C*C*A*C*T*A*+C* | 109161 | 18 |
| | CTA | | +T*+A | | |
| 4358 | GACTGGATCATAGCA | A574356Hi | +G*+A*+C*T*G*G*A*T*C*A*T*A*G*C*A*+A* | 109849 | 18 |
| | AAC | | +A*+C | | |
| 4359 | GGAGGTGGCAAAGCA | A574357H | +G*+G*+A*G*G*T*G*G*C*A*A*A*G*C*A*C* | 109977 | 19 |
| | CCAC | | +C*+A*+C | | |
| 4360 | GGGTGGTGGAGGTG | A574358H | +G*+G*+G*T*G*G*T*G*G*A*G*G*T*G*G*C* | 109984 | 19 |
| | GCAAA | | +A*+A*+A | | |
| 4361 | TCAAACTGGGGTGGT | A574359H | +T*+C*+A*A*A*C*T*G*G*G*G*T*G*G*T*G*+ | 109992 | 19 |
| | GGAG | | G*+A*+G | | |
| 4362 | ATCAAACTGGGGTGG | A574360H | +A*+T*+C*A*A*A*C*T*G*G*G*G*T*G*G*T*+ | 109993 | 19 |
| | TGGA | | G*+G*+A | | |
| 4363 | TATCAAACTGGGGTG | A574361H | +T*+A*+T*C*A*A*A*C*T*G*G*G*G*T*G*G*+ | 109994 | 19 |
| | GTGG | | T*+G*+G | | |
| 4364 | GATATCAAACTGGGG | A574362H | +G*+A*+T*A*T*C*A*A*A*C*T*G*G*G*G*T*+ | 109996 | 19 |
| | TGGT | | G*+G*+T | | |
| 4365 | CCAGCAAAGATATCA | A574363H | +C*+C*+A*G*C*A*A*A*G*A*T*A*T*C*A*A*+ | 110004 | 19 |
| | AACT | | A*+C*+T | | |
| 4366 | GCTGCCAGCAAAGAT | A574364H | +G*+C*+T*G*C*C*A*G*C*A*A*A*G*A*T*A*+ | 110008 | 19 |
| | ATCA | | T*+C*+A | | |
| 4367 | GCTGCCAGCAAAGAT | A574365H | +G*+C*+T*G*C*C*A*G*C*A*A*A*G*A*T*+A* | 110009 | 18 |
| | ATC | | +T*+C | | |
| 4368 | AGCTGCCAGCAAAGA | A574366H | +A*+G*+C*T*G*C*C*A*G*C*A*A*A*G*A*T*+ | 110009 | 19 |
| | TATC | | A*+T*+C | | |
| 4369 | CAGCTGCCAGCAAAG | A574367H | +C*+A*+G*C*T*G*C*C*A*G*C*A*A*A*G*A*+ | 110010 | 19 |
| | ATAT | | T*+A*+T | | |
| 4370 | ACCAACATCCGGGGA | A574368H | +A*+C*+C*A*A*C*A*T*C*C*G*G*G*G*A*C*+ | 110032 | 19 |
| | CGAA | | G*+A*+A | | |
| 4371 | ACCAACATCCGGGGA | A574369H | +A*+C*+C*A*A*C*A*T*C*C*G*G*G*G*A*+C* | 110033 | 18 |
| | CGA | | +G*+A | | |
| 4372 | CACCAACATCCGGGG | A574370H | +C*+A*+C*C*A*A*C*A*T*C*C*G*G*G*G*A*+ | 110033 | 19 |
| | ACGA | | C*+G*+A | | |
| 4373 | ACCAACATCCGGGGA | A574371H | +A*+C*+C*A*A*C*A*T*C*C*G*G*G*G*+A*+ | 110034 | 17 |
| | CG | | C*+G | | |
| 4374 | CACCAACATCCGGGG | A574372H | +C*+A*+C*C*A*A*C*A*T*C*C*G*G*G*G*+A* | 110034 | 18 |
| | ACG | | +C*+G | | |
| 4375 | TCACCAACATCCGGG | A574373H | +T*+C*+A*C*C*A*A*C*A*T*C*C*G*G*G*G*+ | 110034 | 19 |
| | GACG | | A*+C*+G | | |
| 4376 | ATCACCAACATCCGG | A574374H | +A*+T*+C*A*C*C*A*A*C*A*T*C*C*G*G*G*+ | 110035 | 19 |
| | GGAC | | G*+A*+C | | |
| 4377 | CTCCATCACCAACAT | A574375H | +C*+T*+C*C*A*T*C*A*C*C*A*A*C*A*T*C*+ | 110039 | 19 |
| | CCGG | | C*+G*+G | | |
| 4378 | CTCCATCACCAACAT | A574376H | +C*+T*+C*C*A*T*C*A*C*C*A*A*C*A*T*+C* | 110040 | 18 |
| | CCG | | +C*+G | | |
| 4379 | ACTCCATCACCAACA | A574377H | +A*+C*+T*C*C*A*T*C*A*C*C*A*A*C*A*T*+ | 110040 | 19 |
| | TCCG | | C*+C*+G | | |
| 4380 | GGCTAACTCCATCAC | A574378H | +G*+G*+C*T*A*A*C*T*C*C*A*T*C*A*C*C*+ | 110045 | 19 |
| | CAAC | | A*+A*+C | | |
| 4381 | GAGGCTAACTCCATC | A574379H | +G*+A*+G*G*C*T*A*A*C*T*C*C*A*T*C*A*+ | 110047 | 19 |
| | ACCA | | C*+C*+A | | |
| 4382 | GGAGGCTAACTCCAT | A574380H | +G*+G*+A*G*G*C*T*A*A*C*T*C*C*A*T*C*+ | 110048 | 19 |
| | CACC | | A*+C*+C | | |
| 4383 | TGGAGGCTAACTCCA | A574381H | +T*+G*+G*A*G*G*C*T*A*A*C*T*C*C*A*T*+ | 110049 | 19 |
| | TCAC | | C*+A*+C | | |
| 4384 | TTGGAGGCTAACTCC | A574382H | +T*+T*+G*G*A*G*G*C*T*A*A*C*T*C*C*A*+ | 110050 | 19 |
| | ATCA | | T*+C*+A | | |
| 4385 | CCTTGGAGGCTAACT | A574383H | +C*+C*+T*T*G*G*A*G*G*C*T*A*A*C*T*C*+ | 110052 | 19 |
| | CCAT | | C*+A*+T | | |
| 4386 | AACCCTTGGAGGCTA | A574384H | +A*+A*+C*C*C*T*T*G*G*A*G*G*C*T*A*A*+ | 110055 | 19 |
| | ACTC | | C*+T*+C | | |
| 4387 | GAACCCTTGGAGGCT | A574385H | +G*+A*+A*C*C*C*T*T*G*G*A*G*G*C*T*A*+ | 110056 | 19 |
| | AACT | | A*+C*+T | | |
| 4388 | GAACCCTTGGAGGCT | A574386H | +G*+A*+A*C*C*C*T*T*G*G*A*G*G*C*T*+A* | 110057 | 18 |
| | AAC | | +A*+C | | |
| 4389 | GGAACCCTTGGAGGC | A574387H | +G*+G*+A*A*C*C*C*T*T*G*G*A*G*G*C*T*+ | 110057 | 19 |
| | TAAC | | A*+A*+C | | |
| 4390 | GGAACCCTTGGAGGC | A574388H | +G*+G*+A*A*C*C*C*T*T*G*G*A*G*G*C*+T* | 110058 | 18 |
| | TAA | | +A*+A | | |
| 4391 | AGGAACCCTTGGAGG | A574389H | +A*+G*+G*A*A*C*C*C*T*T*G*G*A*G*G*C* | 110058 | 19 |
| | CTAA | | +T*+A*+A | | |
| 4392 | AGGAACCCTTGGAGG | A574390H | +A*+G*+G*A*A*C*C*C*T*T*G*G*A*G*G*+C | 110059 | 18 |
| | CTA | | *+T*+A | | |
| 4393 | AAGGAACCCTTGGAG | A574391H | +A*+A*+G*G*A*A*C*C*C*T*T*G*G*A*G*G* | 110059 | 19 |
| | GCTA | | +C*+T*+A | | |
| 4394 | CAAGGAACCCTTGGA | A574392H | +C*+A*+A*G*G*A*A*C*C*C*T*T*G*G*A*G*+ | 110060 | 19 |
| | GGCT | | G*+C*+T | | |
| 4395 | CCAAGGAACCCTTGG | A574393H | +C*+C*+A*A*G*G*A*A*C*C*C*T*T*G*G*A*+ | 110061 | 19 |
| | AGGC | | G*+G*+C | | |
| 4396 | GATCCAAGGAACCCT | A574394H | +G*+A*+T*C*C*A*A*G*G*A*A*C*C*C*T*T*+ | 110064 | 19 |
| | TGGA | | G*+G*+A | | |
| 4397 | CGATCCAAGGAACCC | A574395H | +C*+G*+A*T*C*C*A*A*G*G*A*A*C*C*C*T*+ | 110065 | 19 |
| | TTGG | | T*+G*+G | | |
| 4398 | CGATCCAAGGAACCC | A574396H | +C*+G*+A*T*C*C*A*A*G*G*A*A*C*C*C*+T* | 110066 | 18 |
| | TTG | | +T*+G | | |
| 4399 | GCGATCCAAGGAACC | A574397H | +G*+C*+G*A*T*C*C*A*A*G*G*A*A*C*C*C*+ | 110066 | 19 |
| | CTTG | | T*+T*+G | | |
| 4400 | GCGATCCAAGGAACC | A574398H | +G*+C*+G*A*T*C*C*A*A*G*G*A*A*C*C*+C* | 110067 | 18 |
| | CTT | | +T*+T | | |
| 4401 | GGCGATCCAAGGAAC | A574399H | +G*+G*+C*G*A*T*C*C*A*A*G*G*A*A*C*C* | 110067 | 19 |
| | CCTT | | +C*+T*+T | | |
| 4402 | AGGCGATCCAAGGAA | A574400H | +A*+G*+G*C*G*A*T*C*C*A*A*G*G*A*A*C* | 110068 | 19 |
| | CCCT | | +C*+C*+T | | |
| 4403 | CAGGCGATCCAAGGA | A574401H | +C*+A*+G*G*C*G*A*T*C*C*A*A*G*G*A*A* | 110069 | 19 |
| | ACCC | | +C*+C*+C | | |
| 4404 | CAGGCGATCCAAGGA | A574402H | +C*+A*+G*G*C*G*A*T*C*C*A*A*G*G*A*+A | 110070 | 18 |
| | ACC | | *+C*+C | | |
| 4405 | GCAGGCGATCCAAGG | A574403H | +G*+C*+A*G*G*C*G*A*T*C*C*A*A*G*G*A* | 110070 | 19 |
| | AACC | | +A*+C*+C | | |
| 4406 | GCAGGCGATCCAAGG | A574404H | +G*+C*+A*G*G*C*G*A*T*C*C*A*A*G*G*+A | 110071 | 18 |
| | AAC | | *+A*+C | | |
| 4407 | AGCAGGCGATCCAAG | A574405H | +A*+G*+C*A*G*G*C*G*A*T*C*C*A*A*G*G* | 110071 | 19 |
| | GAAC | | +A*+A*+C | | |
| 4408 | GCAGGCGATCCAAGG | A574406H | +G*+C*+A*G*G*C*G*A*T*C*C*A*A*G*+G*+ | 110072 | 17 |
| | AA | | A*+A | | |
| 4409 | AGCAGGCGATCCAAG | A574407H | +A*+G*+C*A*G*G*C*G*A*T*C*C*A*A*G*+G | 110072 | 18 |
| | GAA | | *+A*+A | | |
| 4410 | AAGCAGGCGATCCAA | A574408H | +A*+A*+G*C*A*G*G*C*G*A*T*C*C*A*A*G* | 110072 | 19 |
| | GGAA | | +G*+A*+A | | |
| 4411 | GAAGCAGGCGATCCA | A574409H | +G*+A*+A*G*C*A*G*G*C*G*A*T*C*C*A*A* | 110073 | 19 |
| | AGGA | | +G*+G*+A | | |
| 4412 | GAAGCAGGCGATCCA | A574410H | +G*+A*+A*G*C*A*G*G*C*G*A*T*C*C*A*+A | 110074 | 18 |
| | AGG | | *+G*+G | | |
| 4413 | TGAAGCAGGCGATCC | A574411H | +T*+G*+A*A*G*C*A*G*G*C*G*A*T*C*C*A*+ | 110074 | 19 |
| | AAGG | | A*+G*+G | | |
| 4414 | TGAAGCAGGCGATCC | A574412H | +T*+G*+A*A*G*C*A*G*G*C*G*A*T*C*C*+A* | 110075 | 18 |
| | AAG | | +A*+G | | |
| 4415 | CTGAAGCAGGCGATC | A574413H | +C*+T*+G*A*A*G*C*A*G*G*C*G*A*T*C*C*+ | 110075 | 19 |
| | CAAG | | A*+A*+G | | |
| 4416 | TGAAGCAGGCGATCC | A574414H | +T*+G*+A*A*G*C*A*G*G*C*G*A*T*C*+C*+ | 110076 | 17 |
| | AA | | A*+A | | |
| 4417 | CTGAAGCAGGCGATC | A574415H | +C*+T*+G*A*A*G*C*A*G*G*C*G*A*T*C*+C* | 110076 | 18 |
| | CAA | | +A*+A | | |
| 4418 | GCTGAAGCAGGCGAT | A574416H | +G*+C*+T*G*A*A*G*C*A*G*G*C*G*A*T*C* | 110076 | 19 |
| | CCAA | | +C*+A*+A | | |
| 4419 | GCTGAAGCAGGCGAT | A574417H | +G*+C*+T*G*A*A*G*C*A*G*G*C*G*A*T*+C | 110077 | 18 |
| | CCA | | *+C*+A | | |
| 4420 | TGCTGAAGCAGGCGA | A574418H | +T*+G*+C*T*G*A*A*G*C*A*G*G*C*G*A*T*+ | 110077 | 19 |
| | TCCA | | C*+C*+A | | |
| 4421 | CCTGCTGAAGCAGGC | A574419H | +C*+C*+T*G*C*T*G*A*A*G*C*A*G*G*C*G* | 110079 | 19 |
| | GATC | | +A*+T*+C | | |
| 4422 | TCCTGCTGAAGCAGG | A574420H | +T*+C*+C*T*G*C*T*G*A*A*G*C*A*G*G*C*+ | 110080 | 19 |
| | CGAT | | G*+A*+T | | |
| 4423 | TCCTGCTGAAGCAGG | A574421H | +T*+C*+C*T*G*C*T*G*A*A*G*C*A*G*G*+C* | 110081 | 18 |
| | CGA | | +G*+A | | |
| 4424 | GTCCTGCTGAAGCAG | A574422H | +G*+T*+C*C*T*G*C*T*G*A*A*G*C*A*G*G*+ | 110081 | 19 |
| | GCGA | | C*+G*+A | | |
| 4425 | GTCCTGCTGAAGCAG | A574423H | +G*+T*+C*C*T*G*C*T*G*A*A*G*C*A*G*+G* | 110082 | 18 |
| | GCG | | +C*+G | | |
| 4426 | TGTCCTGCTGAAGCA | A574424H | +T*+G*+T*C*C*T*G*C*T*G*A*A*G*C*A*G*+ | 110082 | 19 |
| | GGCG | | G*+C*+G | | |
| 4427 | TTGTCCTGCTGAAGC | A574425H | +T*+T*+G*T*C*C*T*G*C*T*G*A*A*G*C*A*+ | 110083 | 19 |
| | AGGC | | G*+G*+C | | |
| 4428 | TTGTCCTGCTGAAGC | A574426H | +T*+T*+G*T*C*C*T*G*C*T*G*A*A*G*C*+A* | 110084 | 18 |
| | AGG | | +G*+G | | |
| 4429 | TTTGTCCTGCTGAAG | A574427H | +T*+T*+T*G*T*C*C*T*G*C*T*G*A*A*G*C*+ | 110084 | 19 |
| | CAGG | | A*+G*+G | | |
| 4430 | TGGCTTTGTCCTGCT | A574428H | +T*+G*+G*C*T*T*T*G*T*C*C*T*G*C*T*G*+ | 110088 | 19 |
| | GAAG | | A*+A*+G | | |
| 4431 | CTGGCTTTGTCCTGC | A574429H | +C*+T*+G*G*C*T*T*T*G*T*C*C*T*G*C*T*+ | 110089 | 19 |
| | TGAA | | G*+A*+A | | |
| 4432 | TAGTGAGGCTGGCTT | A574430H | +T*+A*+G*T*G*A*G*G*C*T*G*G*C*T*T*T*+ | 110097 | 19 |
| | TGTC | | G*+T*+C | | |
| 4433 | CTAGTGAGGCTGGCT | A574431H | +C*+T*+A*G*T*G*A*G*G*C*T*G*G*C*T*T*+ | 110098 | 19 |
| | TTGT | | T*+G*+T | | |
| 4434 | TCTAGTGAGGCTGGC | A574432H | +T*+C*+T*A*G*T*G*A*G*G*C*T*G*G*C*T*+ | 110099 | 19 |
| | TTTG | | T*+T*+G | | |
| 4435 | TTCTAGTGAGGCTGG | A574433H | +T*+T*+C*T*A*G*T*G*A*G*G*C*T*G*G*C*+ | 110100 | 19 |
| | CTTT | | T*+T*+T | | |
| 4436 | GTTCTAGTGAGGCTG | A574434H | +G*+T*+T*C*T*A*G*T*G*A*G*G*C*T*G*G*+ | 110101 | 19 |
| | GCTT | | C*+T*+T | | |
| 4437 | GGTTCTAGTGAGGCT | A574435H | +G*+G*+T*T*C*T*A*G*T*G*A*G*G*C*T*G*+ | 110102 | 19 |
| | GGCT | | G*+C*+T | | |
| 4438 | GGTTCTAGTGAGGCT | A574436H | +G*+G*+T*T*C*T*A*G*T*G*A*G*G*C*T*+G* | 110103 | 18 |
| | GGC | | +G*+C | | |
| 4439 | GGGTTCTAGTGAGGC | A574437H | +G*+G*+G*T*T*C*T*A*G*T*G*A*G*G*C*T*+ | 110103 | 19 |
| | TGGC | | G*+G*+C | | |
| 4440 | GGGTTCTAGTGAGGC | A574438H | +G*+G*+G*T*T*C*T*A*G*T*G*A*G*G*C*+T* | 110104 | 18 |
| | TGG | | +G*+G | | |
| 4441 | AGGGTTCTAGTGAGG | A574439H | +A*+G*+G*G*T*T*C*T*A*G*T*G*A*G*G*C*+ | 110104 | 19 |
| | CTGG | | T*+G*+G | | |
| 4442 | TAGGGTTCTAGTGAG | A574440H | +T*+A*+G*G*G*T*T*C*T*A*G*T*G*A*G*G*+ | 110105 | 19 |
| | GCTG | | C*+T*+G | | |
| 4443 | GCTGTAGGGTTCTAG | A574441H | +G*+C*+T*G*T*A*G*G*G*T*T*C*T*A*G*T*+ | 110109 | 19 |
| | TGAG | | G*+A*+G | | |
| 4444 | GCTGTAGGGTTCTAG | A574442H | +G*+C*+T*G*T*A*G*G*G*T*T*C*T*A*G*+T* | 110110 | 18 |
| | TGA | | +G*+A | | |
| 4445 | TGCTGTAGGGTTCTA | A574443H | +T*+G*+C*T*G*T*A*G*G*G*T*T*C*T*A*G*+ | 110110 | 19 |
| | GTGA | | T*+G*+A | | |
| 4446 | GTGCTGTAGGGTTCT | A574444H | +G*+T*+G*C*T*G*T*A*G*G*G*T*T*C*T*A*+ | 110111 | 19 |
| | AGTG | | G*+T*+G | | |
| 4447 | CCTGTGCTGTAGGGT | A574445H | +C*+C*+T*G*T*G*C*T*G*T*A*G*G*G*T*T*+ | 110114 | 19 |
| | TCTA | | C*+T*+A | | |
| 4448 | CCTGTGCTGTAGGGT | A574446H | +C*+C*+T*G*T*G*C*T*G*T*A*G*G*G*T*+T* | 110115 | 18 |
| | TCT | | +C*+T | | |
| 4449 | TCCTGTGCTGTAGGG | A574447H | +T*+C*+C*T*G*T*G*C*T*G*T*A*G*G*G*T*+ | 110115 | 19 |
| | TTCT | | T*+C*+T | | |
| 4450 | TCCTGTGCTGTAGGG | A574448H | +T*+C*+C*T*G*T*G*C*T*G*T*A*G*G*G*+T* | 110116 | 18 |
| | TTC | | +T*+C | | |
| 4451 | ATCCTGTGCTGTAGG | A574449H | +A*+T*+C*C*T*G*T*G*C*T*G*T*A*G*G*G*+ | 110116 | 19 |
| | GTTC | | T*+T*+C | | |
| 4452 | CAATCCTGTGCTGTA | A574450H | +C*+A*+A*T*C*C*T*G*T*G*C*T*G*T*A*G*+ | 110118 | 19 |
| | GGGT | | G*+G*+T | | |
| 4453 | GCAATCCTGTGCTGT | A574451H | +G*+C*+A*A*T*C*C*T*G*T*G*C*T*G*T*A*+ | 110119 | 19 |
| | AGGG | | G*+G*+G | | |
| 4454 | TGCAATCCTGTGCTG | A574452H | +T*+G*+C*A*A*T*C*C*T*G*T*G*C*T*G*T*+ | 110120 | 19 |
| | TAGG | | A*+G*+G | | |
| 4455 | TGCAATCCTGTGCTG | A574453H | +T*+G*+C*A*A*T*C*C*T*G*T*G*C*T*G*+T* | 110121 | 18 |
| | TAG | | +A*+G | | |
| 4456 | GTGCAATCCTGTGCT | A574454H | +G*+T*+G*C*A*A*T*C*C*T*G*T*G*C*T*G*+ | 110121 | 19 |
| | GTAG | | T*+A*+G | | |
| 4457 | GTGCAATCCTGTGCT | A574455H | +G*+T*+G*C*A*A*T*C*C*T*G*T*G*C*T*+G* | 110122 | 18 |
| | GTA | | +T*+A | | |
| 4458 | AGTGCAATCCTGTGC | A574456H | +A*+G*+T*G*C*A*A*T*C*C*T*G*T*G*C*T*+ | 110122 | 19 |
| | TGTA | | G*+T*+A | | |
| 4459 | GGAGTGCAATCCTGT | A574457H | +G*+G*+A*G*T*G*C*A*A*T*C*C*T*G*T*G*+ | 110124 | 19 |
| | GCTG | | C*+T*+G | | |
| 4460 | GGAGTGCAATCCTGT | A574458H | +G*+G*+A*G*T*G*C*A*A*T*C*C*T*G*T*+G* | 110125 | 18 |
| | GCT | | +C*+T | | |
| 4461 | TGGAGTGCAATCCTG | A574459H | +T*+G*+G*A*G*T*G*C*A*A*T*C*C*T*G*T*+ | 110125 | 19 |
| | TGCT | | G*+C*+T | | |
| 4462 | TTACTACTTGCTATG | A574460Hi | +T*+T*+A*C*T*A*C*T*T*G*C*T*A*T*G*+A* | 110351 | 18 |
| | AGG | | +G*+G | | |
| 4463 | GAAGGCAGATCAGTA | A574461Hi | +G*+A*+A*G*G*C*A*G*A*T*C*A*G*T*A*+C* | 110416 | 18 |
| | CAG | | +A*+G | | |
| 4464 | GGAATCATGTAATTG | A574462Hi | +G*+G*+A*A*T*C*A*T*G*T*A*A*T*T*G*+A* | 110475 | 18 |
| | ACA | | +C*+A | | |
| 4465 | TATGGACTCATCTTT | A574463Hi | +T*+A*+T*G*G*A*C*T*C*A*T*C*T*T*T*+C* | 110497 | 18 |
| | CCT | | +C*+T | | |
| 4466 | GAGAAGTATGGACTC | A574464Hi | +G*+A*+G*A*A*G*T*A*T*G*G*A*C*T*C*+A* | 110503 | 18 |
| | ATC | | +T*+C | | |
| 4467 | TGAGAAGTATGGACT | A574465Hi | +T*+G*+A*G*A*A*G*T*A*T*G*G*A*C*T*+C* | 110504 | 18 |
| | CAT | | +A*+T | | |
| 4468 | CCTTGAGAAGTATGG | A574466Hi | +C*+C*+T*T*G*A*G*A*A*G*T*A*T*G*G*+A* | 110507 | 18 |
| | ACT | | +C*+T | | |
| 4469 | TTAGATTCTGATGGC | A574467Hi | +T*+T*+A*G*A*T*T*C*T*G*A*T*G*G*C*+A* | 111419 | 18 |
| | AAG | | +A*+G | | |
| 4470 | TACTTAGATTCTGAT | A574468Hi | +T*+A*+C*T*T*A*G*A*T*T*C*T*G*A*T*+G* | 111422 | 18 |
| | GGC | | +G*+C | | |
| 4471 | GTACTTAGATTCTGA | A574469Hi | +G*+T*+A*C*T*T*A*G*A*T*T*C*T*G*A*+T* | 111423 | 18 |
| | TGG | | +G*+G | | |
| 4472 | AAGGCATATTCATAG | A574470Hi | +A*+A*+G*G*C*A*T*A*T*T*C*A*T*A*G*+C* | 111625 | 18 |
| | CAT | | +A*+T | | |
| 4473 | GAGTTAGTTAAATGA | A574471Hi | +G*+A*+G*T*T*A*G*T*T*A*A*A*T*G*A*+A* | 111838 | 18 |
| | AGG | | +G*+G | | |
| 4474 | GGTGTTTCGAACAGT | A574472Hi | +G*+G*+T*G*T*T*T*C*G*A*A*C*A*G*T*+G* | 111932 | 18 |
| | GTT | | +T*+T | | |
| 4475 | GGTGTTTCGAACAGT | A574473Hi | +G*+G*+T*G*T*T*T*C*G*A*A*C*A*G*+T*+G | 111933 | 17 |
| | GT | | *+T | | |
| 4476 | TGCTGGTAGAAGATT | A574474Hi | +T*+G*+C*T*G*G*T*A*G*A*A*G*A*T*T*+G* | 112135 | 18 |
| | GAT | | +A*+T | | |
| 4477 | GTGCTGGTAGAAGAT | A574475Hi | +G*+T*+G*C*T*G*G*T*A*G*A*A*G*A*T*+T* | 112136 | 18 |
| | TGA | | +G*+A | | |
| 4478 | TGCTGGTAGAAGATT | A574476Hi | +T*+G*+C*T*G*G*T*A*G*A*A*G*A*T*+T*+G | 112136 | 17 |
| | GA | | *+A | | |
| 4479 | AGTGCTGGTAGAAGA | A574477Hi | +A*+G*+T*G*C*T*G*G*T*A*G*A*A*G*A*+T* | 112137 | 18 |
| | TTG | | +T*+G | | |
| 4480 | TAGTGCTGGTAGAAG | A574478Hi | +T*+A*+G*T*G*C*T*G*G*T*A*G*A*A*G*+A* | 112138 | 18 |
| | ATT | | +T*+T | | |
| 4481 | TTAGTGCTGGTAGAA | A574479Hi | +T*+T*+A*G*T*G*C*T*G*G*T*A*G*A*A*+G* | 112139 | 18 |
| | GAT | | +A*+T | | |
| 4482 | TCACTCTAAACAGCT | A574480Hi | +T*+C*+A*C*T*C*T*A*A*A*C*A*G*C*T*+C* | 112250 | 18 |
| | CCA | | +C*+A | | |
| 4483 | GTCACTCTAAACAGC | A574481Hi | +G*+T*+C*A*C*T*C*T*A*A*A*C*A*G*C*+T* | 112251 | 18 |
| | TCC | | +C*+C | | |
| 4484 | GAGCATGTAACGTGA | A574482Hi | +G*+A*+G*C*A*T*G*T*A*A*C*G*T*G*+A*+ | 112282 | 17 |
| | AG | | A*+G | | |
| 4485 | TACCAAGGCAGTACA | A574483Hi | +T*+A*+C*C*A*A*G*G*C*A*G*T*A*C*A*+T* | 112448 | 18 |
| | TTC | | +T*+C | | |
| 4486 | TGATGTACCAAGGCA | A574484Hi | +T*+G*+A*T*G*T*A*C*C*A*A*G*G*C*A*+G* | 112453 | 18 |
| | GTA | | +T*+A | | |
| 4487 | GTGATGTACCAAGGC | A574485Hi | +G*+T*+G*A*T*G*T*A*C*C*A*A*G*G*C*+A* | 112454 | 18 |
| | AGT | | +G*+T | | |
| 4488 | GGTGATGTACCAAGG | A574486Hi | +G*+G*+T*G*A*T*G*T*A*C*C*A*A*G*G*+C* | 112455 | 18 |
| | CAG | | +A*+G | | |
| 4489 | AGCTAACACTTCATG | A574487Hi | +A*+G*+C*T*A*A*C*A*C*T*T*C*A*T*G*+A* | 112993 | 18 |
| | ACA | | +C*+A | | |
| 4490 | GTGTCACAATCCAGA | A574488Hi | +G*+T*+G*T*C*A*C*A*A*T*C*C*A*G*A*+A* | 113245 | 18 |
| | AGG | | +G*+G | | |
| 4491 | GCCAGTCATTTAGTA | A574489Hi | +G*+C*+C*A*G*T*C*A*T*T*T*A*G*T*A*+T* | 113295 | 18 |
| | TAC | | +A*+C | | |
| 4492 | GGCCAGTCATTTAGT | A574490Hi | +G*+G*+C*C*A*G*T*C*A*T*T*T*A*G*T*+A* | 113296 | 18 |
| | ATA | | +T*+A | | |
| 4493 | ATGTACTAGGTATCT | A574491Hi | +A*+T*+G*T*A*C*T*A*G*G*T*A*T*C*T*+A* | 113316 | 18 |
| | ATG | | +T*+G | | |
| 4494 | GCTTAAAGTAAACTT | A574492Hi | +G*+C*+T*T*A*A*A*G*T*A*A*A*C*T*T*+T* | 113389 | 18 |
| | TAT | | +A*+T | | |
| 4495 | TCATAGCTTAAAGTA | A574493Hi | +T*+C*+A*T*A*G*C*T*T*A*A*A*G*T*A*+A* | 113394 | 18 |
| | AAC | | +A*+C | | |
| 4496 | AACCATGACTATTGT | A574494Hi | +A*+A*+C*C*A*T*G*A*C*T*A*T*T*G*T*+C* | 113502 | 18 |
| | CAA | | +A*+A | | |
| 4497 | TAATCTTAAGAAGTG | A574495Hi | +T*+A*+A*T*C*T*T*A*A*G*A*A*G*T*G*+C* | 113619 | 18 |
| | CAG | | +A*+G | | |
| 4498 | AGATAATATCTTGGT | A574496Hi | +A*+G*+A*T*A*A*T*A*T*C*T*T*G*G*T*+T* | 113660 | 18 |
| | TAG | | +A*+G | | |
| 4499 | ATACTTTCCAAAGGC | A574497Hi | +A*+T*+A*C*T*T*T*C*C*A*A*A*G*G*C*+T* | 113722 | 18 |
| | TAA | | +A*+A | | |
| 4500 | CCAAATCCTTTATAA | A574498Hi | +C*+C*+A*A*A*T*C*C*T*T*T*A*T*A*A*+G* | 113782 | 18 |
| | GAC | | +A*+C | | |
| 4501 | CCTTCACAGTAAGTA | A574499Hi | +C*+C*+T*T*C*A*C*A*G*T*A*A*G*T*A*+A* | 113927 | 18 |
| | ATG | | +T*+G | | |
| 4502 | TGCTTATTCGTGACT | A574500Hi | +T*+G*+C*T*T*A*T*T*C*G*T*G*A*C*T*+G* | 114215 | 18 |
| | GTT | | +T*+T | | |
| 4503 | TTGCTTATTCGTGAC | A574501Hi | +T*+T*+G*C*T*T*A*T*T*C*G*T*G*A*C*+T* | 114216 | 18 |
| | TGT | | +G*+T | | |
| 4504 | TGCTTATTCGTGACT | A574502Hi | +T*+G*+C*T*T*A*T*T*C*G*T*G*A*C*+T*+G | 114216 | 17 |
| | GT | | *+T | | |
| 4505 | TTTGCTTATTCGTGA | A574503Hi | +T*+T*+T*G*C*T*T*A*T*T*C*G*T*G*A*+C* | 114217 | 18 |
| | CTG | | +T*+G | | |
| 4506 | CTTTGCTTATTCGTG | A574504Hi | +C*+T*+T*T*G*C*T*T*A*T*T*C*G*T*+G*+A | 114219 | 17 |
| | AC | | *+C | | |
| 4507 | ATGATGTCAAATTGA | A574505Hi | +A*+T*+G*A*T*G*T*C*A*A*A*T*T*G*A*+C* | 114321 | 18 |
| | CTA | | +T*+A | | |
| 4508 | GTCTTATCCATTCTG | A574506Hi | +G*+T*+C*T*T*A*T*C*C*A*T*T*C*T*G*+G* | 114435 | 18 |
| | GAA | | +A*+A | | |
| 4509 | GGTCTTATCCATTCT | A574507Hi | +G*+G*+T*C*T*T*A*T*C*C*A*T*T*C*T*+G* | 114436 | 18 |
| | GGA | | +G*+A | | |
| 4510 | CTGTTTGAGTATATA | A574508Hi | +C*+T*+G*T*T*T*G*A*G*T*A*T*A*T*A*+A* | 114678 | 18 |
| | ATT | | +T*+T | | |
| 4511 | GGTATATAATCATGG | A574509H | +G*+G*+T*A*T*A*T*A*A*T*C*A*T*G*G*C*+ | 115308 | 19 |
| | CTGA | | T*+G*+A | | |
| 4512 | GGTATATAATCATGG | A574510H | +G*+G*+T*A*T*A*T*A*A*T*C*A*T*G*G*+C* | 115309 | 18 |
| | CTG | | +T*+G | | |
| 4513 | CGGTATATAATCATG | A574511H | +C*+G*+G*T*A*T*A*T*A*A*T*C*A*T*G*G*+ | 115309 | 19 |
| | GCTG | | C*+T*+G | | |
| 4514 | CGGTATATAATCATG | A574512H | +C*+G*+G*T*A*T*A*T*A*A*T*C*A*T*G*+G* | 115310 | 18 |
| | GCT | | +C*+T | | |
| 4515 | TCGGTATATAATCAT | A574513H | +T*+C*+G*G*T*A*T*A*T*A*A*T*C*A*T*G*+ | 115310 | 19 |
| | GGCT | | G*+C*+T | | |
| 4516 | TCGGTATATAATCAT | A574514H | +T*+C*+G*G*T*A*T*A*T*A*A*T*C*A*T*+G* | 115311 | 18 |
| | GGC | | +G*+C | | |
| 4517 | CTCGGTATATAATCA | A574515H | +C*+T*+C*G*G*T*A*T*A*T*A*A*T*C*A*T*+ | 115311 | 19 |
| | TGGC | | G*+G*+C | | |
| 4518 | TCTCGGTATATAATC | A574516H | +T*+C*+T*C*G*G*T*A*T*A*T*A*A*T*C*A*+ | 115312 | 19 |
| | ATGG | | T*+G*+G | | |
| 4519 | TCTCGGTATATAATC | A574517H | +T*+C*+T*C*G*G*T*A*T*A*T*A*A*T*C*+A* | 115313 | 18 |
| | ATG | | +T*+G | | |
| 4520 | GTCTCGGTATATAAT | A574518H | +G*+T*+C*T*C*G*G*T*A*T*A*T*A*A*T*C*+ | 115313 | 19 |
| | CATG | | A*+T*+G | | |
| 4521 | AAAAGCAGCACATTG | A574519H | +A*+A*+A*A*G*C*A*G*C*A*C*A*T*T*G*T*+ | 115339 | 19 |
| | TGGG | | G*+G*+G | | |
| 4522 | GAAAAGCAGCACATT | A574520H | +G*+A*+A*A*A*G*C*A*G*C*A*C*A*T*T*G*+ | 115340 | 19 |
| | GTGG | | T*+G*+G | | |
| 4523 | GTGAAAAGCAGCACA | A574521H | +G*+T*+G*A*A*A*A*G*C*A*G*C*A*C*A*T*+ | 115342 | 19 |
| | TTGT | | T*+G*+T | | |
| 4524 | GATACAGTGTGAAAA | A574522H | +G*+A*+T*A*C*A*G*T*G*T*G*A*A*A*A*G*+ | 115350 | 19 |
| | GCAG | | C*+A*+G | | |
| 4525 | TGGGATACAGTGTGA | A574523H | +T*+G*+G*G*A*T*A*C*A*G*T*G*T*G*A*A*+ | 115353 | 19 |
| | AAAG | | A*+A*+G | | |
| 4526 | CATTGGGATACAGTG | A574524H | +C*+A*+T*T*G*G*G*A*T*A*C*A*G*T*G*T*+ | 115356 | 19 |
| | TGAA | | G*+A*+A | | |
| 4527 | GCATTGGGATACAGT | A574525H | +G*+C*+A*T*T*G*G*G*A*T*A*C*A*G*T*G*+ | 115357 | 19 |
| | GTGA | | T*+G*+A | | |
| 4528 | GCATTGGGATACAGT | A574526H | +G*+C*+A*T*T*G*G*G*A*T*A*C*A*G*T*+G* | 115358 | 18 |
| | GTG | | +T*+G | | |
| 4529 | AGCATTGGGATACAG | A574527H | +A*+G*+C*A*T*T*G*G*G*A*T*A*C*A*G*T*+ | 115358 | 19 |
| | TGTG | | G*+T*+G | | |
| 4530 | AGCATTGGGATACAG | A574528H | +A*+G*+C*A*T*T*G*G*G*A*T*A*C*A*G*+T* | 115359 | 18 |
| | TGT | | +G*+T | | |
| 4531 | CAGCATTGGGATACA | A574529H | +C*+A*+G*C*A*T*T*G*G*G*A*T*A*C*A*G*+ | 115359 | 19 |
| | GTGT | | T*+G*+T | | |
| 4532 | GCAGCATTGGGATAC | A574530H | +G*+C*+A*G*C*A*T*T*G*G*G*A*T*A*C*A*+ | 115360 | 19 |
| | AGTG | | G*+T*+G | | |
| 4533 | GCAGCATTGGGATAC | A574531H | +G*+C*+A*G*C*A*T*T*G*G*G*A*T*A*C*+A* | 115361 | 18 |
| | AGT | | +G*+T | | |
| 4534 | GGCAGCATTGGGATA | A574532H | +G*+G*+C*A*G*C*A*T*T*G*G*G*A*T*A*C*+ | 115361 | 19 |
| | CAGT | | A*+G*+T | | |
| 4535 | GGCAGCATTGGGATA | A574533H | +G*+G*+C*A*G*C*A*T*T*G*G*G*A*T*A*+C* | 115362 | 18 |
| | CAG | | +A*+G | | |
| 4536 | TGGCAGCATTGGGAT | A574534H | +T*+G*+G*C*A*G*C*A*T*T*G*G*G*A*T*A*+ | 115362 | 19 |
| | ACAG | | C*+A*+G | | |
| 4537 | TGGCAGCATTGGGAT | A574535H | +T*+G*+G*C*A*G*C*A*T*T*G*G*G*A*T*+A* | 115363 | 18 |
| | ACA | | +C*+A | | |
| 4538 | ATGGCAGCATTGGGA | A574536H | +A*+T*+G*G*C*A*G*C*A*T*T*G*G*G*A*T*+ | 115363 | 19 |
| | TACA | | A*+C*+A | | |
| 4539 | TGGCAGCATTGGGAT | A574537H | +T*+G*+G*C*A*G*C*A*T*T*G*G*G*A*+T*+ | 115364 | 17 |
| | AC | | A*+C | | |
| 4540 | ATGGCAGCATTGGGA | A574538H | +A*+T*+G*G*C*A*G*C*A*T*T*G*G*G*A*+T* | 115364 | 18 |
| | TAC | | +A*+C | | |
| 4541 | GATGGCAGCATTGGG | A574539H | +G*+A*+T*G*G*C*A*G*C*A*T*T*G*G*G*A* | 115364 | 19 |
| | ATAC | | +T*+A*+C | | |
| 4542 | TGATGGCAGCATTGG | A574540H | +T*+G*+A*T*G*G*C*A*G*C*A*T*T*G*G*G*+ | 115365 | 19 |
| | GATA | | A*+T*+A | | |
| 4543 | TGATGGCAGCATTGG | A574541H | +T*+G*+A*T*G*G*C*A*G*C*A*T*T*G*G*+G* | 115366 | 18 |
| | GAT | | +A*+T | | |
| 4544 | ATGATGGCAGCATTG | A574542H | +A*+T*+G*A*T*G*G*C*A*G*C*A*T*T*G*G*+ | 115366 | 19 |
| | GGAT | | G*+A*+T | | |
| 4545 | AATGATGGCAGCATT | A574543H | +A*+A*+T*G*A*T*G*G*C*A*G*C*A*T*T*G*+ | 115367 | 19 |
| | GGGA | | G*+G*+A | | |
| 4546 | CTTTGCAATGATGGC | A574544H | +C*+T*+T*T*G*C*A*A*T*G*A*T*G*G*C*A*+ | 115373 | 19 |
| | AGCA | | G*+C*+A | | |
| 4547 | CTTTGCAATGATGGC | A574545H | +C*+T*+T*T*G*C*A*A*T*G*A*T*G*G*C*+A* | 115374 | 18 |
| | AGC | | +G*+C | | |
| 4548 | TCTTTGCAATGATGG | A574546H | +T*+C*+T*T*T*G*C*A*A*T*G*A*T*G*G*C*+ | 115374 | 19 |
| | CAGC | | A*+G*+C | | |
| 4549 | CAATCTTTGCAATGA | A574547H | +C*+A*+A*T*C*T*T*T*G*C*A*A*T*G*A*T*+ | 115377 | 19 |
| | TGGC | | G*+G*+C | | |
| 4550 | GTCAGCAATCTTTGC | A574548H | +G*+T*+C*A*G*C*A*A*T*C*T*T*T*G*C*A*+ | 115382 | 19 |
| | AATG | | A*+T*+G | | |
| 4551 | GTAGTCAGCAATCTT | A574549H | +G*+T*+A*G*T*C*A*G*C*A*A*T*C*T*T*T*+ | 115385 | 19 |
| | TGCA | | G*+C*+A | | |
| 4552 | CGTAGTCAGCAATCT | A574550H | +C*+G*+T*A*G*T*C*A*G*C*A*A*T*C*T*T*+ | 115386 | 19 |
| | TTGC | | T*+G*+C | | |
| 4553 | CCGTAGTCAGCAATC | A574551H | +C*+C*+G*T*A*G*T*C*A*G*C*A*A*T*C*T*+ | 115387 | 19 |
| | TTTG | | T*+T*+G | | |
| 4554 | CCGTAGTCAGCAATC | A574552H | +C*+C*+G*T*A*G*T*C*A*G*C*A*A*T*C*+T* | 115388 | 18 |
| | TTT | | +T*+T | | |
| 4555 | GCCGTAGTCAGCAAT | A574553H | +G*+C*+C*G*T*A*G*T*C*A*G*C*A*A*T*C*+ | 115388 | 19 |
| | CTTT | | T*+T*+T | | |
| 4556 | TGCCGTAGTCAGCAA | A574554H | +T*+G*+C*C*G*T*A*G*T*C*A*G*C*A*A*T*+ | 115389 | 19 |
| | TCTT | | C*+T*+T | | |
| 4557 | TGCCGTAGTCAGCAA | A574555H | +T*+G*+C*C*G*T*A*G*T*C*A*G*C*A*A*+T* | 115390 | 18 |
| | TCT | | +C*+T | | |
| 4558 | ATGCCGTAGTCAGCA | A574556H | +A*+T*+G*C*C*G*T*A*G*T*C*A*G*C*A*A*+ | 115390 | 19 |
| | ATCT | | T*+C*+T | | |
| 4559 | GCAATGCCGTAGTCA | A574557H | +G*+C*+A*A*T*G*C*C*G*T*A*G*T*C*A*G*+ | 115393 | 19 |
| | GCAA | | C*+A*+A | | |
| 4560 | GAGCAATGCCGTAGT | A574558H | +G*+A*+G*C*A*A*T*G*C*C*G*T*A*G*T*C*+ | 115395 | 19 |
| | CAGC | | A*+G*+C | | |
| 4561 | GAGCAATGCCGTAGT | A574559H | +G*+A*+G*C*A*A*T*G*C*C*G*T*A*G*T*+C* | 115396 | 18 |
| | CAG | | +A*+G | | |
| 4562 | TGAGCAATGCCGTAG | A574560H | +T*+G*+A*G*C*A*A*T*G*C*C*G*T*A*G*T*+ | 115396 | 19 |
| | TCAG | | C*+A*+G | | |
| 4563 | GAGCAATGCCGTAGT | A574561H | +G*+A*+G*C*A*A*T*G*C*C*G*T*A*G*+T*+ | 115397 | 17 |
| | CA | | C*+A | | |
| 4564 | TGAGCAATGCCGTAG | A574562H | +T*+G*+A*G*C*A*A*T*G*C*C*G*T*A*G*+T* | 115397 | 18 |
| | TCA | | +C*+A | | |
| 4565 | CTGAGCAATGCCGTA | A574563H | +C*+T*+G*A*G*C*A*A*T*G*C*C*G*T*A*G*+ | 115397 | 19 |
| | GTCA | | T*+C*+A | | |
| 4566 | TGAGCAATGCCGTAG | A574564H | +T*+G*+A*G*C*A*A*T*G*C*C*G*T*A*+G*+T | 115398 | 17 |
| | TC | | *+C | | |
| 4567 | CTGAGCAATGCCGTA | A574565H | +C*+T*+G*A*G*C*A*A*T*G*C*C*G*T*A*+G* | 115398 | 18 |
| | GTC | | +T*+C | | |
| 4568 | ACTGAGCAATGCCGT | A574566H | +A*+C*+T*G*A*G*C*A*A*T*G*C*C*G*T*A*+ | 115398 | 19 |
| | AGTC | | G*+T*+C | | |
| 4569 | AGTACTGAGCAATGC | A574567H | +A*+G*+T*A*C*T*G*A*G*C*A*A*T*G*C*C*+ | 115401 | 19 |
| | CGTA | | G*+T*+A | | |
| 4570 | AGTACTGAGCAATGC | A574568H | +A*+G*+T*A*C*T*G*A*G*C*A*A*T*G*C*+C* | 115402 | 18 |
| | CGT | | +G*+T | | |
| 4571 | CAGTACTGAGCAATG | A574569H | +C*+A*+G*T*A*C*T*G*A*G*C*A*A*T*G*C*+ | 115402 | 19 |
| | CCGT | | C*+G*+T | | |
| 4572 | AGTACTGAGCAATGC | A574570H | +A*+G*+T*A*C*T*G*A*G*C*A*A*T*G*+C*+C | 115403 | 17 |
| | CG | | *+G | | |
| 4573 | CAGTACTGAGCAATG | A574571H | +C*+A*+G*T*A*C*T*G*A*G*C*A*A*T*G*+C* | 115403 | 18 |
| | CCG | | +C*+G | | |
| 4574 | GCAGTACTGAGCAAT | A574572H | +G*+C*+A*G*T*A*C*T*G*A*G*C*A*A*T*G*+ | 115403 | 19 |
| | GCCG | | C*+C*+G | | |
| 4575 | AGCAGTACTGAGCAA | A574573H | +A*+G*+C*A*G*T*A*C*T*G*A*G*C*A*A*T*+ | 115404 | 19 |
| | TGCC | | G*+C*+C | | |
| 4576 | CCCCATTCTACAGCA | A574574H | +C*+C*+C*C*A*T*T*C*T*A*C*A*G*C*A*G*+ | 115415 | 19 |
| | GTAC | | T*+A*+C | | |
| 4577 | TCCCCATTCTACAGC | A574575H | +T*+C*+C*C*C*A*T*T*C*T*A*C*A*G*C*A*+ | 115416 | 19 |
| | AGTA | | G*+T*+A | | |
| 4578 | GGTTATCCATCCTGA | A574576Hi | +G*+G*+T*T*A*T*C*C*A*T*C*C*T*G*A*+A* | 115489 | 18 |
| | AGA | | +G*+A | | |
| 4579 | GGTCAGTGGTTATCC | A574577Hi | +G*+G*+T*C*A*G*T*G*G*T*T*A*T*C*C*+A* | 115496 | 18 |
| | ATC | | +T*+C | | |
| 4580 | AGGTCAGTGGTTATC | A574578Hi | +A*+G*+G*T*C*A*G*T*G*G*T*T*A*T*C*+C* | 115497 | 18 |
| | CAT | | +A*+T | | |
| 4581 | TGAGGTCAGTGGTTA | A574579Hi | +T*+G*+A*G*G*T*C*A*G*T*G*G*T*T*A*+T* | 115499 | 18 |
| | TCC | | +C*+C | | |
| 4582 | CTTCTGAACTCACAT | A574580Hi | +C*+T*+T*C*T*G*A*A*C*T*C*A*C*A*T*+C* | 115515 | 18 |
| | CTG | | +T*+G | | |
| 4583 | GAATTTGATTTGCCT | A574581Hi | +G*+A*+A*T*T*T*G*A*T*T*T*G*C*C*T*+C* | 115797 | 18 |
| | CAC | | +A*+C | | |
| 4584 | GAGACAACATGTCTG | A574582Hi | +G*+A*+G*A*C*A*A*C*A*T*G*T*C*T*G*+T* | 116122 | 18 |
| | TAC | | +A*+C | | |
| 4585 | TTAATTGAGTTGCAA | A574583Hi | +T*+T*+A*A*T*T*G*A*G*T*T*G*C*A*A*+T* | 116278 | 18 |
| | TCA | | +C*+A | | |
| 4586 | GGTGATTTAATTGAG | A574584Hi | +G*+G*+T*G*A*T*T*T*A*A*T*T*G*A*G*+T* | 116284 | 18 |
| | TTG | | +T*+G | | |
| 4587 | TCAGCCAGAACATAC | A574585Hi | +T*+C*+A*G*C*C*A*G*A*A*C*A*T*A*C*+A* | 116513 | 18 |
| | ACT | | +C*+T | | |
| 4588 | GGCCAATTACTGAGT | A574586Hi | +G*+G*+C*C*A*A*T*T*A*C*T*G*A*G*T*+G* | 116725 | 18 |
| | GAT | | +A*+T | | |
| 4589 | GCCAAATATCTCTGG | A574587Hi | +G*+C*+C*A*A*A*T*A*T*C*T*C*T*G*G*+A* | 116773 | 18 |
| | ATC | | +T*+C | | |
| 4590 | GTCCAGTCCACCTTC | A574588Hi | +G*+T*+C*C*A*G*T*C*C*A*C*C*T*T*C*+A* | 117035 | 18 |
| | ATG | | +T*+G | | |
| 4591 | GAAACTGAGTGGTCC | A574589Hi | +G*+A*+A*A*C*T*G*A*G*T*G*G*T*C*C*+A* | 117046 | 18 |
| | AGT | | +G*+T | | |
| 4592 | ATGAGTCTGAACCAA | A574590Hi | +A*+T*+G*A*G*T*C*T*G*A*A*C*C*A*A*+G* | 117238 | 18 |
| | GGT | | +G*+T | | |
| 4593 | GCTTGGTATGAAGAA | A574591Hi | +G*+C*+T*T*G*G*T*A*T*G*A*A*G*A*A*+T* | 117323 | 18 |
| | TCA | | +C*+A | | |
| 4594 | CATCGCTCATGCTCT | A574592Hi | +C*+A*+T*C*G*C*T*C*A*T*G*C*T*C*T*+G* | 117897 | 18 |
| | GGA | | +G*+A | | |
| 4595 | GCACATCGCTCATGC | A574593Hi | +G*+C*+A*C*A*T*C*G*C*T*C*A*T*G*C*+T* | 117900 | 18 |
| | TCT | | +C*+T | | |
| 4596 | GCTATCACAGTGCTG | A574594Hi | +G*+C*+T*A*T*C*A*C*A*G*T*G*C*T*G*+G* | 118115 | 18 |
| | GAC | | +A*+C | | |
| 4597 | CCAAGTTATTTAAAG | A574595Hi | +C*+C*+A*A*G*T*T*A*T*T*T*A*A*A*G*+G* | 118227 | 18 |
| | GTG | | +T*+G | | |
| 4598 | GTAGAGTATGTTCCA | A574596Hi | +G*+T*+A*G*A*G*T*A*T*G*T*T*C*C*A*+T* | 118473 | 18 |
| | TAA | | +A*+A | | |
| 4599 | AGTAGAGTATGTTCC | A574597Hi | +A*+G*+T*A*G*A*G*T*A*T*G*T*T*C*C*+A* | 118474 | 18 |
| | ATA | | +T*+A | | |
| 4600 | ATGAGTAGAGTATGT | A574598Hi | +A*+T*+G*A*G*T*A*G*A*G*T*A*T*G*T*+T* | 118477 | 18 |
| | TCC | | +C*+C | | |
| 4601 | GTTCTTGATTGCACT | A574599Hi | +G*+T*+T*C*T*T*G*A*T*T*G*C*A*C*T*+A* | 118849 | 18 |
| | AAC | | +A*+C | | |
| 4602 | CTAGTGTTCTTGATT | A574600Hi | +C*+T*+A*G*T*G*T*T*C*T*T*G*A*T*T*+G* | 118854 | 18 |
| | GCA | | +C*+A | | |
| 4603 | CTAGTGTTCTTGATT | A574601Hi | +C*+T*+A*G*T*G*T*T*C*T*T*G*A*T*+T*+G | 118855 | 17 |
| | GC | | *+C | | |
| 4604 | GCAAAGTCAAGAGTA | A574602Hi | +G*+C*+A*A*A*G*T*C*A*A*G*A*G*T*A*+C* | 119475 | 18 |
| | CTT | | +T*+T | | |
| 4605 | AGCTCAGCAGTCCTC | A574603Hi | +A*+G*+C*T*C*A*G*C*A*G*T*C*C*T*C*+A* | 120017 | 18 |
| | ACC | | +C*+C | | |
| 4606 | AATTCATAGCCTTCA | A574604Hi | +A*+A*+T*T*C*A*T*A*G*C*C*T*T*C*A*+A* | 120095 | 18 |
| | AGG | | +G*+G | | |
| 4607 | CTTTCTAAGATGATA | A574605Hi | +C*+T*+T*T*C*T*A*A*G*A*T*G*A*T*A*+T* | 120966 | 18 |
| | TGC | | +G*+C | | |
| 4608 | GATCACTTTCTAAGA | A574606Hi | +G*+A*+T*C*A*C*T*T*T*C*T*A*A*G*A*+T* | 120971 | 18 |
| | TGA | | +G*+A | | |
| 4609 | TTCTAACACTCTCATT | A574607Hi | +T*+T*+C*T*A*A*C*A*C*T*C*T*C*A*T*+T* | 121272 | 18 |
| | AT | | +A*+T | | |
| 4610 | TATTTCCAGGACAGT | A574608Hi | +T*+A*+T*T*T*C*C*A*G*G*A*C*A*G*T*+G* | 121361 | 18 |
| | GTT | | +T*+T | | |
| 4611 | GGATAAACATTCAAT | A574609Hi | +G*+G*+A*T*A*A*A*C*A*T*T*C*A*A*T*+G* | 121461 | 18 |
| | GGT | | +G*+T | | |
| 4612 | GTGGAGTTCTAACAG | A574610Hi | +G*+T*+G*G*A*G*T*T*C*T*A*A*C*A*G*+C* | 122179 | 18 |
| | CAA | | +A*+A | | |
| 4613 | CATTCCAGGATAATG | A574611Hi | +C*+A*+T*T*C*C*A*G*G*A*T*A*A*T*G*+A* | 122235 | 18 |
| | AGT | | +G*+T | | |
| 4614 | AGGTTTATCCAATTA | A574612Hi | +A*+G*+G*T*T*T*A*T*C*C*A*A*T*T*A*+A* | 122605 | 18 |
| | ATA | | +T*+A | | |
| 4615 | TGGTTTGTGCACATT | A574613Hi | +T*+G*+G*T*T*T*G*T*G*C*A*C*A*T*T*+T* | 123023 | 18 |
| | TGT | | +G*+T | | |
| 4616 | CACAGATGGTTTGTG | A574614Hi | +C*+A*+C*A*G*A*T*G*G*T*T*T*G*T*G*+C* | 123029 | 18 |
| | CAC | | +A*+C | | |
| 4617 | ATGTCCTCACAGATG | A574615Hi | +A*+T*+G*T*C*C*T*C*A*C*A*G*A*T*G*+G* | 123036 | 18 |
| | GTT | | +T*+T | | |
| 4618 | TATGTCCTCACAGAT | A574616Hi | +T*+A*+T*G*T*C*C*T*C*A*C*A*G*A*T*+G* | 123037 | 18 |
| | GGT | | +G*+T | | |
| 4619 | TTGATCTCCACTAAG | A574617Hi | +T*+T*+G*A*T*C*T*C*C*A*C*T*A*A*+G*+A | 123230 | 17 |
| | AG | | *+G | | |
| 4620 | TAAGCCTAAGACTGG | A574618Hi | +T*+A*+A*G*C*C*T*A*A*G*A*C*T*G*G*+T* | 124361 | 18 |
| | TTA | | +T*+A | | |
| 4621 | GCTAAGCCTAAGACT | A574619Hi | +G*+C*+T*A*A*G*C*C*T*A*A*G*A*C*T*+G* | 124363 | 18 |
| | GGT | | +G*+T | | |
| 4622 | TGGCACACAATTGGC | A574620Hi | +T*+G*+G*C*A*C*A*C*A*A*T*T*G*G*C*+T* | 124651 | 18 |
| | TGA | | +G*+A | | |
| 4623 | GTGGCACACAATTGG | A574621Hi | +G*+T*+G*G*C*A*C*A*C*A*A*T*T*G*G*+C* | 124652 | 18 |
| | CTG | | +T*+G | | |
| 4624 | AGACAGTCTTAGTAA | A574622Hi | +A*+G*+A*C*A*G*T*C*T*T*A*G*T*A*A*+A* | 125199 | 18 |
| | AGG | | +G*+G | | |
| 4625 | TACTCTTGGATGCTT | A574623Hi | +T*+A*+C*T*C*T*T*G*G*A*T*G*C*T*T*+T* | 125261 | 18 |
| | TGT | | +G*+T | | |
| 4626 | GTATACTCTTGGATG | A574624Hi | +G*+T*+A*T*A*C*T*C*T*T*G*G*A*T*G*+C* | 125264 | 18 |
| | CTT | | +T*+T | | |
| 4627 | GACATTTGTCTGCCA | A574625Hi | +G*+A*+C*A*T*T*T*G*T*C*T*G*C*C*A*+A* | 125302 | 18 |
| | AGA | | +G*+A | | |
| 4628 | GCAAATCTTCCAACT | A574626Hi | +G*+C*+A*A*A*T*C*T*T*C*C*A*A*C*T*+A* | 125469 | 18 |
| | AAA | | +A*+A | | |
| 4629 | CTTTCAGATAATATG | A574627Hi | +C*+T*+T*T*C*A*G*A*T*A*A*T*A*T*G*+G* | 125502 | 18 |
| | GTG | | +T*+G | | |
| 4630 | TAGTTGTACTTGACT | A574628Hi | +T*+A*+G*T*T*G*T*A*C*T*T*G*A*C*T*+A* | 125644 | 18 |
| | ATT | | +T*+T | | |
| 4631 | TCCTGTCTTGCCTAG | A574629Hi | +T*+C*+C*T*G*T*C*T*T*G*C*C*T*A*G*+T* | 125924 | 18 |
| | TAT | | +A*+T | | |
| 4632 | GTAGTTGAAAGGAAC | A574630Hi | +G*+T*+A*G*T*T*G*A*A*A*G*G*A*A*C*+A* | 126151 | 18 |
| | AGG | | +G*+G | | |
| 4633 | TCTAGTAGTTGAAAG | A574631Hi | +T*+C*+T*A*G*T*A*G*T*T*G*A*A*A*G*+G* | 126155 | 18 |
| | GAA | | +A*+A | | |
| 4634 | GAGCTTCTAGTAGTT | A574632Hi | +G*+A*+G*C*T*T*C*T*A*G*T*A*G*T*T*+G* | 126160 | 18 |
| | GAA | | +A*+A | | |
| 4635 | GTTGAGCTTCTAGTA | A574633Hi | +G*+T*+T*G*A*G*C*T*T*C*T*A*G*T*A*+G* | 126163 | 18 |
| | GTT | | +T*+T | | |
| 4636 | GTTGAGCTTCTAGTA | A574634Hi | +G*+T*+T*G*A*G*C*T*T*C*T*A*G*T*+A*+G | 126164 | 17 |
| | GT | | *+T | | |
| 4637 | AGTTGAGCTTCTAGT | A574635Hi | +A*+G*+T*T*G*A*G*C*T*T*C*T*A*G*T*+A* | 126164 | 18 |
| | AGT | | +G*+T | | |
| 4638 | TGTTATAGCTCTGTC | A574636Hi | +T*+G*+T*T*A*T*A*G*C*T*C*T*G*T*C*+A* | 126447 | 18 |
| | AAG | | +A*+G | | |
| 4639 | GCAGATTGCAGCCAA | A574637Hi | +G*+C*+A*G*A*T*T*G*C*A*G*C*C*A*A*+T* | 127160 | 18 |
| | TGA | | +G*+A | | |
| 4640 | AGCAGATTGCAGCCA | A574638Hi | +A*+G*+C*A*G*A*T*T*G*C*A*G*C*C*A*+A* | 127161 | 18 |
| | ATG | | +T*+G | | |
| 4641 | CAGCAGATTGCAGCC | A574639Hi | +C*+A*+G*C*A*G*A*T*T*G*C*A*G*C*C*+A* | 127162 | 18 |
| | AAT | | +A*+T | | |
| 4642 | TTGGCTACTATAGTT | A574640Hi | +T*+T*+G*G*C*T*A*C*T*A*T*A*G*T*T*+C* | 127267 | 18 |
| | CAG | | +A*+G | | |
| 4643 | GTTGGCTACTATAGT | A574641Hi | +G*+T*+T*G*G*C*T*A*C*T*A*T*A*G*T*+T* | 127268 | 18 |
| | TCA | | +C*+A | | |
| 4644 | ATATTCTACTATCAG | A574642H | +A*+T*+A*T*T*C*T*A*C*T*A*T*C*A*G*C*+ | 129306 | 19 |
| | CAAC | | A*+A*+C | | |
| 4645 | GCACAATATTCTACT | A574643H | +G*+C*+A*C*A*A*T*A*T*T*C*T*A*C*T*A*+ | 129311 | 19 |
| | ATCA | | T*+C*+A | | |
| 4646 | GCACAATATTCTACT | A574644H | +G*+C*+A*C*A*A*T*A*T*T*C*T*A*C*T*+A* | 129312 | 18 |
| | ATC | | +T*+C | | |
| 4647 | AGCACAATATTCTAC | A574645H | +A*+G*+C*A*C*A*A*T*A*T*T*C*T*A*C*T*+ | 129312 | 19 |
| | TATC | | A*+T*+C | | |
| 4648 | AAGCACAATATTCTA | A574646H | +A*+A*+G*C*A*C*A*A*T*A*T*T*C*T*A*C*+ | 129313 | 19 |
| | CTAT | | T*+A*+T | | |
| 4649 | TAAGCACAATATTCT | A574647H | +T*+A*+A*G*C*A*C*A*A*T*A*T*T*C*T*A*+ | 129314 | 19 |
| | ACTA | | C*+T*+A | | |
| 4650 | CTAAGCACAATATTC | A574648H | +C*+T*+A*A*G*C*A*C*A*A*T*A*T*T*C*T*+ | 129315 | 19 |
| | TACT | | A*+C*+T | | |
| 4651 | CTAAGCACAATATTC | A574649H | +C*+T*+A*A*G*C*A*C*A*A*T*A*T*T*C*+T* | 129316 | 18 |
| | TAC | | +A*+C | | |
| 4652 | GCTAAGCACAATATT | A574650H | +G*+C*+T*A*A*G*C*A*C*A*A*T*A*T*T*C*+ | 129316 | 19 |
| | CTAC | | T*+A*+C | | |
| 4653 | AGGCTAAGCACAATA | A574651H | +A*+G*+G*C*T*A*A*G*C*A*C*A*A*T*A*T*+ | 129318 | 19 |
| | TTCT | | T*+C*+T | | |
| 4654 | AGGCTAAGCACAATA | A574652H | +A*+G*+G*C*T*A*A*G*C*A*C*A*A*T*A*+T* | 129319 | 18 |
| | TTC | | +T*+C | | |
| 4655 | AAGGCTAAGCACAAT | A574653H | +A*+A*+G*G*C*T*A*A*G*C*A*C*A*A*T*A*+ | 129319 | 19 |
| | ATTC | | T*+T*+C | | |
| 4656 | CAAGGCTAAGCACAA | A574654H | +C*+A*+A*G*G*C*T*A*A*G*C*A*C*A*A*T*+ | 129320 | 19 |
| | TATT | | A*+T*+T | | |
| 4657 | CCAAGGCTAAGCACA | A574655H | +C*+C*+A*A*G*G*C*T*A*A*G*C*A*C*A*A*+ | 129321 | 19 |
| | ATAT | | T*+A*+T | | |
| 4658 | ACCAAGGCTAAGCAC | A574656H | +A*+C*+C*A*A*G*G*C*T*A*A*G*C*A*C*A*+ | 129322 | 19 |
| | AATA | | A*+T*+A | | |
| 4659 | ACCAAGGCTAAGCAC | A574657H | +A*+C*+C*A*A*G*G*C*T*A*A*G*C*A*C*+A* | 129323 | 18 |
| | AAT | | +A*+T | | |
| 4660 | CACCAAGGCTAAGCA | A574658H | +C*+A*+C*C*A*A*G*G*C*T*A*A*G*C*A*C*+ | 129323 | 19 |
| | CAAT | | A*+A*+T | | |
| 4661 | CACCAAGGCTAAGCA | A574659H | +C*+A*+C*C*A*A*G*G*C*T*A*A*G*C*A*+C* | 129324 | 18 |
| | CAA | | +A*+A | | |
| 4662 | GCACCAAGGCTAAGC | A574660H | +G*+C*+A*C*C*A*A*G*G*C*T*A*A*G*C*A*+ | 129324 | 19 |
| | ACAA | | C*+A*+A | | |
| 4663 | GCACCAAGGCTAAGC | A574661H | +G*+C*+A*C*C*A*A*G*G*C*T*A*A*G*C*+A* | 129325 | 18 |
| | ACA | | +C*+A | | |
| 4664 | TGCACCAAGGCTAAG | A574662H | +T*+G*+C*A*C*C*A*A*G*G*C*T*A*A*G*C*+ | 129325 | 19 |
| | CACA | | A*+C*+A | | |
| 4665 | TGCACCAAGGCTAAG | A574663H | +T*+G*+C*A*C*C*A*A*G*G*C*T*A*A*G*+C* | 129326 | 18 |
| | CAC | | +A*+C | | |
| 4666 | ATGCACCAAGGCTAA | A574664H | +A*+T*+G*C*A*C*C*A*A*G*G*C*T*A*A*G*+ | 129326 | 19 |
| | GCAC | | C*+A*+C | | |
| 4667 | GATGCACCAAGGCTA | A574665H | +G*+A*+T*G*C*A*C*C*A*A*G*G*C*T*A*A*+ | 129327 | 19 |
| | AGCA | | G*+C*+A | | |
| 4668 | GATGCACCAAGGCTA | A574666H | +G*+A*+T*G*C*A*C*C*A*A*G*G*C*T*A*+A* | 129328 | 18 |
| | AGC | | +G*+C | | |
| 4669 | AGATGCACCAAGGCT | A574667H | +A*+G*+A*T*G*C*A*C*C*A*A*G*G*C*T*A*+ | 129328 | 19 |
| | AAGC | | A*+G*+C | | |
| 4670 | AGATGCACCAAGGCT | A574668H | +A*+G*+A*T*G*C*A*C*C*A*A*G*G*C*T*+A* | 129329 | 18 |
| | AAG | | +A*+G | | |
| 4671 | AAGATGCACCAAGGC | A574669H | +A*+A*+G*A*T*G*C*A*C*C*A*A*G*G*C*T*+ | 129329 | 19 |
| | TAAG | | A*+A*+G | | |
| 4672 | AAGATGCACCAAGGC | A574670H | +A*+A*+G*A*T*G*C*A*C*C*A*A*G*G*C*+T* | 129330 | 18 |
| | TAA | | +A*+A | | |
| 4673 | GAAGATGCACCAAGG | A574671H | +G*+A*+A*G*A*T*G*C*A*C*C*A*A*G*G*C* | 129330 | 19 |
| | CTAA | | +T*+A*+A | | |
| 4674 | GGAAGATGCACCAAG | A574672H | +G*+G*+A*A*G*A*T*G*C*A*C*C*A*A*G*G* | 129331 | 19 |
| | GCTA | | +C*+T*+A | | |
| 4675 | CAGGAAGATGCACCA | A574673H | +C*+A*+G*G*A*A*G*A*T*G*C*A*C*C*A*A*+ | 129333 | 19 |
| | AGGC | | G*+G*+C | | |
| 4676 | AACAGGAAGATGCAC | A574674H | +A*+A*+C*A*G*G*A*A*G*A*T*G*C*A*C*C*+ | 129335 | 19 |
| | CAAG | | A*+A*+G | | |
| 4677 | GAGTACCAGACTGTG | A574675H | +G*+A*+G*T*A*C*C*A*G*A*C*T*G*T*G*T*+ | 129378 | 19 |
| | TCCC | | C*+C*+C | | |
| 4678 | AGAGTACCAGACTGT | A574676H | +A*+G*+A*G*T*A*C*C*A*G*A*C*T*G*T*G*+ | 129379 | 19 |
| | GTCC | | T*+C*+C | | |
| 4679 | AGAGTACCAGACTGT | A574677H | +A*+G*+A*G*T*A*C*C*A*G*A*C*T*G*T*+G* | 129380 | 18 |
| | GTC | | +T*+C | | |
| 4680 | GAGAGTACCAGACTG | A574678H | +G*+A*+G*A*G*T*A*C*C*A*G*A*C*T*G*T*+ | 129380 | 19 |
| | TGTC | | G*+T*+C | | |
| 4681 | GGAGAGTACCAGACT | A574679H | +G*+G*+A*G*A*G*T*A*C*C*A*G*A*C*T*G* | 129381 | 19 |
| | GTGT | | +T*+G*+T | | |
| 4682 | CAGGAGAGTACCAGA | A574680H | +C*+A*+G*G*A*G*A*G*T*A*C*C*A*G*A*C* | 129383 | 19 |
| | CTGT | | +T*+G*+T | | |
| 4683 | CAGGAGAGTACCAGA | A574681H | +C*+A*+G*G*A*G*A*G*T*A*C*C*A*G*A*+C | 129384 | 18 |
| | CTG | | *+T*+G | | |
| 4684 | CCAGGAGAGTACCAG | A574682H | +C*+C*+A*G*G*A*G*A*G*T*A*C*C*A*G*A* | 129384 | 19 |
| | ACTG | | +C*+T*+G | | |
| 4685 | TGACCAGGAGAGTAC | A574683H | +T*+G*+A*C*C*A*G*G*A*G*A*G*T*A*C*C*+ | 129387 | 19 |
| | CAGA | | A*+G*+A | | |
| 4686 | TTGATGACCAGGAGA | A574684H | +T*+T*+G*A*T*G*A*C*C*A*G*G*A*G*A*G*+ | 129391 | 19 |
| | GTAC | | T*+A*+C | | |
| 4687 | TATTGATGACCAGGA | A574685H | +T*+A*+T*T*G*A*T*G*A*C*C*A*G*G*A*G*+ | 129393 | 19 |
| | GAGT | | A*+G*+T | | |
| 4688 | GGTATTGATGACCAG | A574686H | +G*+G*+T*A*T*T*G*A*T*G*A*C*C*A*G*G*+ | 129395 | 19 |
| | GAGA | | A*+G*+A | | |
| 4689 | GGTATTGATGACCAG | A574687H | +G*+G*+T*A*T*T*G*A*T*G*A*C*C*A*G*+G* | 129396 | 18 |
| | GAG | | +A*+G | | |
| 4690 | CGGTATTGATGACCA | A574688H | +C*+G*+G*T*A*T*T*G*A*T*G*A*C*C*A*G*+ | 129396 | 19 |
| | GGAG | | G*+A*+G | | |
| 4691 | TCGGTATTGATGACC | A574689H | +T*+C*+G*G*T*A*T*T*G*A*T*G*A*C*C*A*+ | 129397 | 19 |
| | AGGA | | G*+G*+A | | |
| 4692 | TTCGGTATTGATGAC | A574690H | +T*+T*+C*G*G*T*A*T*T*G*A*T*G*A*C*C*+ | 129398 | 19 |
| | CAGG | | A*+G*+G | | |
| 4693 | TCTTCGGTATTGATG | A574691H | +T*+C*+T*T*C*G*G*T*A*T*T*G*A*T*G*A*+ | 129400 | 19 |
| | ACCA | | C*+C*+A | | |
| 4694 | TCTTCGGTATTGATG | A574692H | +T*+C*+T*T*C*G*G*T*A*T*T*G*A*T*G*+A* | 129401 | 18 |
| | ACC | | +C*+C | | |
| 4695 | ATCTTCGGTATTGAT | A574693H | +A*+T*+C*T*T*C*G*G*T*A*T*T*G*A*T*G*+ | 129401 | 19 |
| | GACC | | A*+C*+C | | |
| 4696 | ATCTTCGGTATTGAT | A574694H | +A*+T*+C*T*T*C*G*G*T*A*T*T*G*A*T*+G* | 129402 | 18 |
| | GAC | | +A*+C | | |
| 4697 | CATCTTCGGTATTGA | A574695H | +C*+A*+T*C*T*T*C*G*G*T*A*T*T*G*A*T*+ | 129402 | 19 |
| | TGAC | | G*+A*+C | | |
| 4698 | CATCTTCGGTATTGA | A574696H | +C*+A*+T*C*T*T*C*G*G*T*A*T*T*G*A*+T* | 129403 | 18 |
| | TGA | | +G*+A | | |
| 4699 | CCATCTTCGGTATTG | A574697H | +C*+C*+A*T*C*T*T*C*G*G*T*A*T*T*G*A*+ | 129403 | 19 |
| | ATGA | | T*+G*+A | | |
| 4700 | CCATCTTCGGTATTG | A574698H | +C*+C*+A*T*C*T*T*C*G*G*T*A*T*T*G*+A* | 129404 | 18 |
| | ATG | | +T*+G | | |
| 4701 | CCCATCTTCGGTATT | A574699H | +C*+C*+C*A*T*C*T*T*C*G*G*T*A*T*T*G*+ | 129404 | 19 |
| | GATG | | A*+T*+G | | |
| 4702 | CCCATCTTCGGTATT | A574700H | +C*+C*+C*A*T*C*T*T*C*G*G*T*A*T*T*+G* | 129405 | 18 |
| | GAT | | +A*+T | | |
| 4703 | TCCCATCTTCGGTAT | A574701H | +T*+C*+C*C*A*T*C*T*T*C*G*G*T*A*T*T*+ | 129405 | 19 |
| | TGAT | | G*+A*+T | | |
| 4704 | CCCATCTTCGGTATT | A574702H | +C*+C*+C*A*T*C*T*T*C*G*G*T*A*T*+T*+G | 129406 | 17 |
| | GA | | *+A | | |
| 4705 | TCCCATCTTCGGTAT | A574703H | +T*+C*+C*C*A*T*C*T*T*C*G*G*T*A*T*+T* | 129406 | 18 |
| | TGA | | +G*+A | | |
| 4706 | TTCCCATCTTCGGTA | A574704H | +T*+T*+C*C*C*A*T*C*T*T*C*G*G*T*A*T*+ | 129406 | 19 |
| | TTGA | | T*+G*+A | | |
| 4707 | TTTCCCATCTTCGGT | A574705H | +T*+T*+T*C*C*C*A*T*C*T*T*C*G*G*T*A*+ | 129407 | 19 |
| | ATTG | | T*+T*+G | | |
| 4708 | TTTTCCCATCTTCGGT | A574706H | +T*+T*+T*T*C*C*C*A*T*C*T*T*C*G*G*T*+ | 129408 | 19 |
| | ATT | | A*+T*+T | | |
| 4709 | TTTTCCCATCTTCGGT | A574707H | +T*+T*+T*T*C*C*C*A*T*C*T*T*C*G*G*+T* | 129409 | 18 |
| | AT | | +A*+T | | |
| 4710 | TTTTTCCCATCTTCGG | A574708H | +T*+T*+T*T*T*C*C*C*A*T*C*T*T*C*G*G*+ | 129409 | 19 |
| | TAT | | T*+A*+T | | |
| 4711 | CTTTTTCCCATCTTCG | A574709H | +C*+T*+T*T*T*T*C*C*C*A*T*C*T*T*C*G*+ | 129410 | 19 |
| | GTA | | G*+T*+A | | |
| 4712 | TCTTTTTCCCATCTTC | A574710H | +T*+C*+T*T*T*T*T*C*C*C*A*T*C*T*T*C*+ | 129411 | 19 |
| | GGT | | G*+G*+T | | |
| 4713 | TTCTAGGGTATGTCT | A574711H | +T*+T*+C*T*A*G*G*G*T*A*T*G*T*C*T*C*+ | 129425 | 19 |
| | CTTT | | T*+T*+T | | |
| 4714 | TTTCTAGGGTATGTC | A574712H | +T*+T*+T*C*T*A*G*G*G*T*A*T*G*T*C*T*+ | 129426 | 19 |
| | TCTT | | C*+T*+T | | |
| 4715 | CATCTTTTCTAGGGT | A574713H | +C*+A*+T*C*T*T*T*T*C*T*A*G*G*G*T*A*+ | 129431 | 19 |
| | ATGT | | T*+G*+T | | |
| 4716 | TCATCTTTTCTAGGG | A574714H | +T*+C*+A*T*C*T*T*T*T*C*T*A*G*G*G*T*+ | 129432 | 19 |
| | TATG | | A*+T*+G | | |
| 4717 | GTCATCTTTTCTAGG | A574715H | +G*+T*+C*A*T*C*T*T*T*T*C*T*A*G*G*G*+ | 129433 | 19 |
| | GTAT | | T*+A*+T | | |
| 4718 | TTGCTTGGAAAAGGA | A574716H | +T*+T*+G*C*T*T*G*G*A*A*A*A*G*G*A*A*+ | 129476 | 19 |
| | ATTG | | T*+T*+G | | |
| 4719 | GTGACCTTCCACAAT | A574717Hi | +G*+T*+G*A*C*C*T*T*C*C*A*C*A*A*T*+T* | 129628 | 18 |
| | TCC | | +C*+C | | |
| 4720 | GAGGTAATACCAGGA | A574718Hi | +G*+A*+G*G*T*A*A*T*A*C*C*A*G*G*A*+G | 129730 | 18 |
| | GCA | | *+C*+A | | |
| 4721 | GTTGAGGTAATACCA | A574719Hi | +G*+T*+T*G*A*G*G*T*A*A*T*A*C*C*A*+G* | 129733 | 18 |
| | GGA | | +G*+A | | |
| 4722 | CACAGTTGAGGTAAT | A574720Hi | +C*+A*+C*A*G*T*T*G*A*G*G*T*A*A*T*+A* | 129737 | 18 |
| | ACC | | +C*+C | | |
| 4723 | TGTTTAAGCCTGGCA | A574721Hi | +T*+G*+T*T*T*A*A*G*C*C*T*G*G*C*A*+C* | 129878 | 18 |
| | CAC | | +A*+C | | |
| 4724 | TCCGAAACTGCCAGT | A574722Hi | +T*+C*+C*G*A*A*A*C*T*G*C*C*A*G*T*+G* | 130190 | 18 |
| | GGA | | +G*+A | | |
| 4725 | TCAACTCCGAAACTG | A574723Hi | +T*+C*+A*A*C*T*C*C*G*A*A*A*C*T*G*+C* | 130195 | 18 |
| | CCA | | +C*+A | | |
| 4726 | CACATTGACAACTGC | A574724Hi | +C*+A*+C*A*T*T*G*A*C*A*A*C*T*G*C*+T* | 130403 | 18 |
| | TCC | | +C*+C | | |
| 4727 | CACATTGACAACTGC | A574725Hi | +C*+A*+C*A*T*T*G*A*C*A*A*C*T*G*+C*+T | 130404 | 17 |
| | TC | | *+C | | |
| 4728 | TCACATTGACAACTG | A574726Hi | +T*+C*+A*C*A*T*T*G*A*C*A*A*C*T*G*+C* | 130404 | 18 |
| | CTC | | +T*+C | | |
| 4729 | CGGTTCCAACCAAAA | A574727H | +C*+G*+G*T*T*C*C*A*A*C*C*A*A*A*A*G*+ | 131132 | 19 |
| | GAAA | | A*+A*+A | | |
| 4730 | CGGTTCCAACCAAAA | A574728H | +C*+G*+G*T*T*C*C*A*A*C*C*A*A*A*A*+G* | 131133 | 18 |
| | GAA | | +A*+A | | |
| 4731 | GCGGTTCCAACCAAA | A574729H | +G*+C*+G*G*T*T*C*C*A*A*C*C*A*A*A*A*+ | 131133 | 19 |
| | AGAA | | G*+A*+A | | |
| 4732 | GCGGTTCCAACCAAA | A574730H | +G*+C*+G*G*T*T*C*C*A*A*C*C*A*A*A*+A* | 131134 | 18 |
| | AGA | | +G*+A | | |
| 4733 | AGCGGTTCCAACCAA | A574731H | +A*+G*+C*G*G*T*T*C*C*A*A*C*C*A*A*A*+ | 131134 | 19 |
| | AAGA | | A*+G*+A | | |
| 4734 | AGCGGTTCCAACCAA | A574732H | +A*+G*+C*G*G*T*T*C*C*A*A*C*C*A*A*+A* | 131135 | 18 |
| | AAG | | +A*+G | | |
| 4735 | CAGCGGTTCCAACCA | A574733H | +C*+A*+G*C*G*G*T*T*C*C*A*A*C*C*A*A*+ | 131135 | 19 |
| | AAAG | | A*+A*+G | | |
| 4736 | TCAGCGGTTCCAACC | A574734H | +T*+C*+A*G*C*G*G*T*T*C*C*A*A*C*C*A*+ | 131136 | 19 |
| | AAAA | | A*+A*+A | | |
| 4737 | TCAGCGGTTCCAACC | A574735H | +T*+C*+A*G*C*G*G*T*T*C*C*A*A*C*C*+A* | 131137 | 18 |
| | AAA | | +A*+A | | |
| 4738 | ATCAGCGGTTCCAAC | A574736H | +A*+T*+C*A*G*C*G*G*T*T*C*C*A*A*C*C*+ | 131137 | 19 |
| | CAAA | | A*+A*+A | | |
| 4739 | TCAGCGGTTCCAACC | A574737H | +T*+C*+A*G*C*G*G*T*T*C*C*A*A*C*+C*+A | 131138 | 17 |
| | AA | | *+A | | |
| 4740 | ATCAGCGGTTCCAAC | A574738H | +A*+T*+C*A*G*C*G*G*T*T*C*C*A*A*C*+C* | 131138 | 18 |
| | CAA | | +A*+A | | |
| 4741 | CATCAGCGGTTCCAA | A574739H | +C*+A*+T*C*A*G*C*G*G*T*T*C*C*A*A*C*+ | 131138 | 19 |
| | CCAA | | C*+A*+A | | |
| 4742 | CATCAGCGGTTCCAA | A574740H | +C*+A*+T*C*A*G*C*G*G*T*T*C*C*A*A*+C* | 131139 | 18 |
| | CCA | | +C*+A | | |
| 4743 | CCATCAGCGGTTCCA | A574741H | +C*+C*+A*T*C*A*G*C*G*G*T*T*C*C*A*A*+ | 131139 | 19 |
| | ACCA | | C*+C*+A | | |
| 4744 | GCCATCAGCGGTTCC | A574742H | +G*+C*+C*A*T*C*A*G*C*G*G*T*T*C*C*A*+ | 131140 | 19 |
| | AACC | | A*+C*+C | | |
| 4745 | GCCATCAGCGGTTCC | A574743H | +G*+C*+C*A*T*C*A*G*C*G*G*T*T*C*C*+A* | 131141 | 18 |
| | AAC | | +A*+C | | |
| 4746 | TGCCATCAGCGGTTC | A574744H | +T*+G*+C*C*A*T*C*A*G*C*G*G*T*T*C*C*+ | 131141 | 19 |
| | CAAC | | A*+A*+C | | |
| 4747 | GCCATCAGCGGTTCC | A574745H | +G*+C*+C*A*T*C*A*G*C*G*G*T*T*C*+C*+A | 131142 | 17 |
| | AA | | *+A | | |
| 4748 | TGCCATCAGCGGTTC | A574746H | +T*+G*+C*C*A*T*C*A*G*C*G*G*T*T*C*+C* | 131142 | 18 |
| | CAA | | +A*+A | | |
| 4749 | TTGCCATCAGCGGTT | A574747H | +T*+T*+G*C*C*A*T*C*A*G*C*G*G*T*T*C*+ | 131142 | 19 |
| | CCAA | | C*+A*+A | | |
| 4750 | TGCCATCAGCGGTTC | A574748H | +T*+G*+C*C*A*T*C*A*G*C*G*G*T*T*+C*+C | 131143 | 17 |
| | CA | | *+A | | |
| 4751 | TTGCCATCAGCGGTT | A574749H | +T*+T*+G*C*C*A*T*C*A*G*C*G*G*T*T*+C* | 131143 | 18 |
| | CCA | | +C*+A | | |
| 4752 | AATTTAGATGTAGAA | A574750Hi | +A*+A*+T*T*T*A*G*A*T*G*T*A*G*A*A*T*+ | 131204 | 19 |
| | TGCA | | G*+C*+A | | |
| 4753 | AATTTATGATTCTTGC | A574751Hi | +A*+A*+T*T*T*A*T*G*A*T*T*C*T*T*G*C*+ | 131285 | 19 |
| | TCT | | T*+C*+T | | |
| 4754 | CATAATTTATGATTCT | A574752Hi | +C*+A*+T*A*A*T*T*T*A*T*G*A*T*T*C*T*+ | 131288 | 19 |
| | TGC | | T*+G*+C | | |
| 4755 | ATACAGTCAATCAGC | A574753Hi | +A*+T*+A*C*A*G*T*C*A*A*T*C*A*G*C*A*+ | 131359 | 19 |
| | AAGT | | A*+G*+T | | |
| 4756 | AATATACAGTCAATC | A574754Hi | +A*+A*+T*A*T*A*C*A*G*T*C*A*A*T*C*A*+ | 131362 | 19 |
| | AGCA | | G*+C*+A | | |
| 4757 | AAATATACAGTCAAT | A574755Hi | +A*+A*+A*T*A*T*A*C*A*G*T*C*A*A*T*C*+ | 131363 | 19 |
| | CAGC | | A*+G*+C | | |
| 4758 | AATATACAGTCAATC | A574756Hi | +A*+A*+T*A*T*A*C*A*G*T*C*A*A*T*C*+A* | 131363 | 18 |
| | AGC | | +G*+C | | |
| 4759 | AAATATACAGTCAAT | A574757Hi | +A*+A*+A*T*A*T*A*C*A*G*T*C*A*A*T*+C* | 131364 | 18 |
| | CAG | | +A*+G | | |
| 4760 | TCTTAGTTCATTCATA | A574758Hi | +T*+C*+T*T*A*G*T*T*C*A*T*T*C*A*T*A*+ | 131381 | 19 |
| | TAA | | T*+A*+A | | |
| 4761 | ATCAGATGTCATTTA | A574759Hi | +A*+T*+C*A*G*A*T*G*T*C*A*T*T*T*A*T*+ | 131405 | 19 |
| | TCAT | | C*+A*+T | | |
| 4762 | ATTATCAATCAGATG | A574760Hi | +A*+T*+T*A*T*C*A*A*T*C*A*G*A*T*G*T*+ | 131412 | 19 |
| | TCAT | | C*+A*+T | | |
| 4763 | ATATTTATAAGGAAT | A574761Hi | +A*+T*+A*T*T*T*A*T*A*A*G*G*A*A*T*T*+ | 131472 | 19 |
| | TATC | | A*+T*+C | | |
| 4764 | GATTTCTTCAATGCTT | A574762Hi | +G*+A*+T*T*T*C*T*T*C*A*A*T*G*C*T*T*+ | 131551 | 19 |
| | AAG | | A*+A*+G | | |
| 4765 | ATGATTTCTTCAATG | A574763Hi | +A*+T*+G*A*T*T*T*C*T*T*C*A*A*T*G*C*+ | 131553 | 19 |
| | CTTA | | T*+T*+A | | |
| 4766 | GTGAATAGATGGAAC | A574764Hi | +G*+T*+G*A*A*T*A*G*A*T*G*G*A*A*C*+C* | 131641 | 18 |
| | CAA | | +A*+A | | |
| 4767 | AGTGAATAGATGGAA | A574765Hi | +A*+G*+T*G*A*A*T*A*G*A*T*G*G*A*A*+C* | 131642 | 18 |
| | CCA | | +C*+A | | |
| 4768 | GAGTGAATAGATGGA | A574766Hi | +G*+A*+G*T*G*A*A*T*A*G*A*T*G*G*A*+A* | 131643 | 18 |
| | ACC | | +C*+C | | |
| 4769 | TTATTGTTACATTCAA | A574767Hi | +T*+T*+A*T*T*G*T*T*A*C*A*T*T*C*A*A*+ | 131778 | 19 |
| | TCT | | T*+C*+T | | |
| 4770 | TACTCGTGAAGCTGA | A574768Hi | +T*+A*+C*T*C*G*T*G*A*A*G*C*T*G*A*T*+ | 132888 | 19 |
| | TGTT | | G*+T*+T | | |
| 4771 | ACTCGTGAAGCTGAT | A574769Hi | +A*+C*+T*C*G*T*G*A*A*G*C*T*G*A*T*+G* | 132888 | 18 |
| | GTT | | +T*+T | | |
| 4772 | CCATCTTAAATGATT | A574770Hi | +C*+C*+A*T*C*T*T*A*A*A*T*G*A*T*T*A*+ | 134155 | 19 |
| | AGAG | | G*+A*+G | | |
| 4773 | CAAATTGAGTATCCA | A574771Hi | +C*+A*+A*A*T*T*G*A*G*T*A*T*C*C*A*C*+ | 134214 | 19 |
| | CAGA | | A*+G*+A | | |
| 4774 | CAAATTGAGTATCCA | A574772Hi | +C*+A*+A*A*T*T*G*A*G*T*A*T*C*C*A*+C* | 134215 | 18 |
| | CAG | | +A*+G | | |
| 4775 | AGTATCTTCAAAGGA | A574773H | +A*+G*+T*A*T*C*T*T*C*A*A*A*G*G*A*G*+ | 134274 | 19 |
| | GCAG | | C*+A*+G | | |
| 4776 | TATTTAGTATCTTCAA | A574774H | +T*+A*+T*T*T*A*G*T*A*T*C*T*T*C*A*A*+ | 134279 | 19 |
| | AGG | | A*+G*+G | | |
| 4777 | TCCTATATTTAGTATC | A574775H | +T*+C*+C*T*A*T*A*T*T*T*A*G*T*A*T*C*+ | 134284 | 19 |
| | TTC | | T*+T*+C | | |
| 4778 | ATGGAGTACTGACAT | A574776H | +A*+T*+G*G*A*G*T*A*C*T*G*A*C*A*T*T*+ | 134300 | 19 |
| | TTCC | | T*+C*+C | | |
| 4779 | AATGGAGTACTGACA | A574777H | +A*+A*+T*G*G*A*G*T*A*C*T*G*A*C*A*T*+ | 134301 | 19 |
| | TTTC | | T*+T*+C | | |
| 4780 | ACACATCAATGGAGT | A574778H | +A*+C*+A*C*A*T*C*A*A*T*G*G*A*G*T*A*+ | 134308 | 19 |
| | ACTG | | C*+T*+G | | |
| 4781 | AACACATCAATGGAG | A574779H | +A*+A*+C*A*C*A*T*C*A*A*T*G*G*A*G*T*+ | 134309 | 19 |
| | TACT | | A*+C*+T | | |
| 4782 | CTCAAACACATCAAT | A574780H | +C*+T*+C*A*A*A*C*A*C*A*T*C*A*A*T*G*+ | 134313 | 19 |
| | GGAG | | G*+A*+G | | |
| 4783 | TCACTCAAACACATC | A574781H | +T*+C*+A*C*T*C*A*A*A*C*A*C*A*T*C*A*+ | 134316 | 19 |
| | AATG | | A*+T*+G | | |
| 4784 | TGGATTCACTCAAAC | A574782H | +T*+G*+G*A*T*T*C*A*C*T*C*A*A*A*C*A*+ | 134321 | 19 |
| | ACAT | | C*+A*+T | | |
| 4785 | GTGGATTCACTCAAA | A574783H | +G*+T*+G*G*A*T*T*C*A*C*T*C*A*A*A*C*+ | 134322 | 19 |
| | CACA | | A*+C*+A | | |
| 4786 | TGTGGATTCACTCAA | A574784H | +T*+G*+T*G*G*A*T*T*C*A*C*T*C*A*A*A*+ | 134323 | 19 |
| | ACAC | | C*+A*+C | | |
| 4787 | TTGTGGATTCACTCA | A574785H | +T*+T*+G*T*G*G*A*T*T*C*A*C*T*C*A*A*+ | 134324 | 19 |
| | AACA | | A*+C*+A | | |
| 4788 | TTGTGGATTCACTCA | A574786H | +T*+T*+G*T*G*G*A*T*T*C*A*C*T*C*A*+A* | 134325 | 18 |
| | AAC | | +A*+C | | |
| 4789 | TTTGTGGATTCACTC | A574787H | +T*+T*+T*G*T*G*G*A*T*T*C*A*C*T*C*A*+ | 134325 | 19 |
| | AAAC | | A*+A*+C | | |
| 4790 | GTTGAATTTGTGGAT | A574788H | +G*+T*+T*G*A*A*T*T*T*G*T*G*G*A*T*T*+ | 134331 | 19 |
| | TCAC | | C*+A*+C | | |
| 4791 | CTCCCCACATTACAT | A574789H | +C*+T*+C*C*C*C*A*C*A*T*T*A*C*A*T*T*+ | 134354 | 19 |
| | TTCT | | T*+C*+T | | |
| 4792 | TCCTCCCCACATTAC | A574790H | +T*+C*+C*T*C*C*C*C*A*C*A*T*T*A*C*A*+ | 134356 | 19 |
| | ATTT | | T*+T*+T | | |
| 4793 | ATCCTCCCCACATTA | A574791H | +A*+T*+C*C*T*C*C*C*C*A*C*A*T*T*A*C*+ | 134357 | 19 |
| | CATT | | A*+T*+T | | |
| 4794 | ATCCTCCCCACATTA | A574792H | +A*+T*+C*C*T*C*C*C*C*A*C*A*T*T*A*+C* | 134358 | 18 |
| | CAT | | +A*+T | | |
| 4795 | CATCCTCCCCACATT | A574793H | +C*+A*+T*C*C*T*C*C*C*C*A*C*A*T*T*A*+ | 134358 | 19 |
| | ACAT | | C*+A*+T | | |
| 4796 | ACATCCTCCCCACAT | A574794H | +A*+C*+A*T*C*C*T*C*C*C*C*A*C*A*T*T*+ | 134359 | 19 |
| | TACA | | A*+C*+A | | |
| 4797 | ACATCCTCCCCACAT | A574795H | +A*+C*+A*T*C*C*T*C*C*C*C*A*C*A*T*+T* | 134360 | 18 |
| | TAC | | +A*+C | | |
| 4798 | CACATCCTCCCCACA | A574796H | +C*+A*+C*A*T*C*C*T*C*C*C*C*A*C*A*T*+ | 134360 | 19 |
| | TTAC | | T*+A*+C | | |
| 4799 | TTTGTGCCACATCCT | A574797H | +T*+T*+T*G*T*G*C*C*A*C*A*T*C*C*T*C*+ | 134367 | 19 |
| | CCCC | | C*+C*+C | | |
| 4800 | AAATCTTTGTGCCAC | A574798H | +A*+A*+A*T*C*T*T*T*G*T*G*C*C*A*C*A*+ | 134372 | 19 |
| | ATCC | | T*+C*+C | | |
| 4801 | AAAATCTTTGTGCCA | A574799H | +A*+A*+A*A*T*C*T*T*T*G*T*G*C*C*A*C*+ | 134373 | 19 |
| | CATC | | A*+T*+C | | |
| 4802 | GAAATCATTAGAAAA | A574800H | +G*+A*+A*A*T*C*A*T*T*A*G*A*A*A*A*G*+ | 134392 | 19 |
| | GGAG | | G*+A*+G | | |
| 4803 | GGTGAAATCATTAGA | A574801H | +G*+G*+T*G*A*A*A*T*C*A*T*T*A*G*A*A*+ | 134395 | 19 |
| | AAAG | | A*+A*+G | | |
| 4804 | AATGGTGAAATCATT | A574802H | +A*+A*+T*G*G*T*G*A*A*A*T*C*A*T*T*A*+ | 134398 | 19 |
| | AGAA | | G*+A*+A | | |
| 4805 | AATGGTGAAATCATT | A574803H | +A*+A*+T*G*G*T*G*A*A*A*T*C*A*T*T*+A* | 134399 | 18 |
| | AGA | | +G*+A | | |
| 4806 | GAATGGTGAAATCAT | A574804H | +G*+A*+A*T*G*G*T*G*A*A*A*T*C*A*T*T*+ | 134399 | 19 |
| | TAGA | | A*+G*+A | | |
| 4807 | TCTGAATGGTGAAAT | A574805H | +T*+C*+T*G*A*A*T*G*G*T*G*A*A*A*T*C*+ | 134402 | 19 |
| | CATT | | A*+T*+T | | |
| 4808 | TCTCAATGAGTTTCT | A574806H | +T*+C*+T*C*A*A*T*G*A*G*T*T*T*C*T*G*+ | 134414 | 19 |
| | GAAT | | A*+A*+T | | |
| 4809 | GTCTCAATGAGTTTC | A574807H | +G*+T*+C*T*C*A*A*T*G*A*G*T*T*T*C*T*+ | 134415 | 19 |
| | TGAA | | G*+A*+A | | |
| 4810 | CTTGTCTCAATGAGT | A574808H | +C*+T*+T*G*T*C*T*C*A*A*T*G*A*G*T*T*+ | 134418 | 19 |
| | TTCT | | T*+C*+T | | |
| 4811 | CTTGTCTCAATGAGT | A574809H | +C*+T*+T*G*T*C*T*C*A*A*T*G*A*G*T*+T* | 134419 | 18 |
| | TTC | | +T*+C | | |
| 4812 | TCTTGTCTCAATGAG | A574810H | +T*+C*+T*T*G*T*C*T*C*A*A*T*G*A*G*T*+ | 134419 | 19 |
| | TTTC | | T*+T*+C | | |
| 4813 | TTCTTGTCTCAATGA | A574811H | +T*+T*+C*T*T*G*T*C*T*C*A*A*T*G*A*G*+ | 134420 | 19 |
| | GTTT | | T*+T*+T | | |
| 4814 | TTCTTGTCTCAATGA | A574812H | +T*+T*+C*T*T*G*T*C*T*C*A*A*T*G*A*+G* | 134421 | 18 |
| | GTT | | +T*+T | | |
| 4815 | GTTCTTGTCTCAATG | A574813H | +G*+T*+T*C*T*T*G*T*C*T*C*A*A*T*G*A*+ | 134421 | 19 |
| | AGTT | | G*+T*+T | | |
| 4816 | GTTCTTGTCTCAATG | A574814H | +G*+T*+T*C*T*T*G*T*C*T*C*A*A*T*G*+A* | 134422 | 18 |
| | AGT | | +G*+T | | |
| 4817 | TGTTCTTGTCTCAAT | A574815H | +T*+G*+T*T*C*T*T*G*T*C*T*C*A*A*T*G*+ | 134422 | 19 |
| | GAGT | | A*+G*+T | | |
| 4818 | TTGTTCTTGTCTCAAT | A574816H | +T*+T*+G*T*T*C*T*T*G*T*C*T*C*A*A*T*+ | 134423 | 19 |
| | GAG | | G*+A*+G | | |
| 4819 | GCTTGTTCTTGTCTC | A574817H | +G*+C*+T*T*G*T*T*C*T*T*G*T*C*T*C*A*+ | 134425 | 19 |
| | AATG | | A*+T*+G | | |
| 4820 | GGCTTGTTCTTGTCT | A574818H | +G*+G*+C*T*T*G*T*T*C*T*T*G*T*C*T*C*+ | 134426 | 19 |
| | CAAT | | A*+A*+T | | |
| 4821 | GTTGGCTTGTTCTTG | A574819H | +G*+T*+T*G*G*C*T*T*G*T*T*C*T*T*G*T*+ | 134429 | 19 |
| | TCTC | | C*+T*+C | | |
| 4822 | GTTGGCTTGTTCTTG | A574820H | +G*+T*+T*G*G*C*T*T*G*T*T*C*T*T*G*+T* | 134430 | 18 |
| | TCT | | +C*+T | | |
| 4823 | AGTTGGCTTGTTCTT | A574821H | +A*+G*+T*T*G*G*C*T*T*G*T*T*C*T*T*G*+ | 134430 | 19 |
| | GTCT | | T*+C*+T | | |
| 4824 | AGTTGGCTTGTTCTT | A574822H | +A*+G*+T*T*G*G*C*T*T*G*T*T*C*T*T*+G* | 134431 | 18 |
| | GTC | | +T*+C | | |
| 4825 | TTATGAATTATCAAC | A574823Hi | +T*+T*+A*T*G*A*A*T*T*A*T*C*A*A*C*A*+ | 134519 | 19 |
| | AGTA | | G*+T*+A | | |
| 4826 | TTCCTTATGAATTATC | A574824Hi | +T*+T*+C*C*T*T*A*T*G*A*A*T*T*A*T*C*+ | 134523 | 19 |
| | AAC | | A*+A*+C | | |
| 4827 | AAGATCTTCCTTATG | A574825Hi | +A*+A*+G*A*T*C*T*T*C*C*T*T*A*T*G*A*+ | 134529 | 19 |
| | AATT | | A*+T*+T | | |
| 4828 | ATACTACTAAGATTA | A574826Hi | +A*+T*+A*C*T*A*C*T*A*A*G*A*T*T*A*C*+ | 134683 | 19 |
| | CAAT | | A*+A*+T | | |
| 4829 | AATACTACTAAGATT | A574827Hi | +A*+A*+T*A*C*T*A*C*T*A*A*G*A*T*T*A*+ | 134684 | 19 |
| | ACAA | | C*+A*+A | | |
| 4830 | GTCTTCCAATTTCAT | A574828Hi | +G*+T*+C*T*T*C*C*A*A*T*T*T*C*A*T*G*+ | 134750 | 19 |
| | GTCT | | T*+C*+T | | |
| 4831 | TGTCTTCCAATTTCAT | A574829Hi | +T*+G*+T*C*T*T*C*C*A*A*T*T*T*C*A*T*+ | 134751 | 19 |
| | GTC | | G*+T*+C | | |
| 4832 | TATCTTTCCTACAATC | A574830Hi | +T*+A*+T*C*T*T*T*C*C*T*A*C*A*A*T*C*+ | 134835 | 19 |
| | AAT | | A*+A*+T | | |
| 4833 | CTATCTTTCCTACAAT | A574831Hi | +C*+T*+A*T*C*T*T*T*C*C*T*A*C*A*A*T*+ | 134836 | 19 |
| | CAA | | C*+A*+A | | |
| 4834 | AATACTTGCTGTCTA | A574832Hi | +A*+A*+T*A*C*T*T*G*C*T*G*T*C*T*A*+T* | 134849 | 18 |
| | TCT | | +C*+T | | |
| 4835 | AGAATACTTGCTGTC | A574833Hi | +A*+G*+A*A*T*A*C*T*T*G*C*T*G*T*C*T*+ | 134850 | 19 |
| | TATC | | A*+T*+C | | |
| 4836 | CAAGAATACTTGCTG | A574834Hi | +C*+A*+A*G*A*A*T*A*C*T*T*G*C*T*G*T*+ | 134852 | 19 |
| | TCTA | | C*+T*+A | | |
| 4837 | GCAAGAATACTTGCT | A574835Hi | +G*+C*+A*A*G*A*A*T*A*C*T*T*G*C*T*G*+ | 134853 | 19 |
| | GTCT | | T*+C*+T | | |
| 4838 | GACAAGCAAGAATAC | A574836Hi | +G*+A*+C*A*A*G*C*A*A*G*A*A*T*A*C*T*+ | 134858 | 19 |
| | TTGC | | T*+G*+C | | |
| 4839 | GAAACTGGTATCACT | A574837Hi | +G*+A*+A*A*C*T*G*G*T*A*T*C*A*C*T*A*+ | 134926 | 19 |
| | AACT | | A*+C*+T | | |
| 4840 | TTGAAACTGGTATCA | A574838Hi | +T*+T*+G*A*A*A*C*T*G*G*T*A*T*C*A*C*+ | 134928 | 19 |
| | CTAA | | T*+A*+A | | |
| 4841 | TGAAACTGGTATCAC | A574839Hi | +T*+G*+A*A*A*C*T*G*G*T*A*T*C*A*C*+T* | 134928 | 18 |
| | TAA | | +A*+A | | |
| 4842 | GTTTGAAACTGGTAT | A574840Hi | +G*+T*+T*T*G*A*A*A*C*T*G*G*T*A*T*C*+ | 134930 | 19 |
| | CACT | | A*+C*+T | | |
| 4843 | TGTTTGAAACTGGTA | A574841Hi | +T*+G*+T*T*T*G*A*A*A*C*T*G*G*T*A*T*+ | 134931 | 19 |
| | TCAC | | C*+A*+C | | |
| 4844 | GTTTGAAACTGGTAT | A574842Hi | +G*+T*+T*T*G*A*A*A*C*T*G*G*T*A*T*+C* | 134931 | 18 |
| | CAC | | +A*+C | | |
| 4845 | CAAACTTGAAAGAGG | A574843Hi | +C*+A*+A*A*C*T*T*G*A*A*A*G*A*G*G*T*+ | 134972 | 19 |
| | TTTA | | T*+T*+A | | |
| 4846 | CAATAGATGGCAAAG | A574844Hi | +C*+A*+A*T*A*G*A*T*G*G*C*A*A*A*G*C*+ | 135103 | 19 |
| | CAAT | | A*+A*+T | | |
| 4847 | TGACTGAAGATGTGA | A574845Hi | +T*+G*+A*C*T*G*A*A*G*A*T*G*T*G*A*A*+ | 135144 | 19 |
| | AAGG | | A*+G*+G | | |
| 4848 | AGATGACTGAAGATG | A574846Hi | +A*+G*+A*T*G*A*C*T*G*A*A*G*A*T*G*T*+ | 135147 | 19 |
| | TGAA | | G*+A*+A | | |
| 4849 | CCTCAGGTAAGAGAT | A574847Hi | +C*+C*+T*C*A*G*G*T*A*A*G*A*G*A*T*G*+ | 135158 | 19 |
| | GACT | | A*+C*+T | | |
| 4850 | ATCCTCAGGTAAGAG | A574848Hi | +A*+T*+C*C*T*C*A*G*G*T*A*A*G*A*G*A*+ | 135160 | 19 |
| | ATGA | | T*+G*+A | | |
| 4851 | AATCCTCAGGTAAGA | A574849Hi | +A*+A*+T*C*C*T*C*A*G*G*T*A*A*G*A*G*+ | 135161 | 19 |
| | GATG | | A*+T*+G | | |
| 4852 | ACAATCCTCAGGTAA | A574850Hi | +A*+C*+A*A*T*C*C*T*C*A*G*G*T*A*A*G*+ | 135163 | 19 |
| | GAGA | | A*+G*+A | | |
| 4853 | AGAATATTACAATCC | A574851Hi | +A*+G*+A*A*T*A*T*T*A*C*A*A*T*C*C*T*+ | 135171 | 19 |
| | TCAG | | C*+A*+G | | |
| 4854 | GGATTTGTGACTCAA | A574852Hi | +G*+G*+A*T*T*T*G*T*G*A*C*T*C*A*A*A*+ | 135307 | 19 |
| | AGCT | | G*+C*+T | | |
| 4855 | ACTGAGAACAGTGAG | A574853Hi | +A*+C*+T*G*A*G*A*A*C*A*G*T*G*A*G*A*+ | 135461 | 19 |
| | AAGT | | A*+G*+T | | |
| 4856 | TGTTAATGTGGCCAC | A574854Hi | +T*+G*+T*T*A*A*T*G*T*G*G*C*C*A*C*A*+ | 135511 | 19 |
| | AGAT | | G*+A*+T | | |
| 4857 | AACTGGATGCTCTTG | A574855Hi | +A*+A*+C*T*G*G*A*T*G*C*T*C*T*T*G*T*+ | 135524 | 19 |
| | TTAA | | T*+A*+A | | |
| 4858 | GAACTGGATGCTCTT | A574856Hi | +G*+A*+A*C*T*G*G*A*T*G*C*T*C*T*T*G*+ | 135525 | 19 |
| | GTTA | | T*+T*+A | | |
| 4859 | GAGAACTGGATGCTC | A574857Hi | +G*+A*+G*A*A*C*T*G*G*A*T*G*C*T*C*T*+ | 135527 | 19 |
| | TTGT | | T*+G*+T | | |
| 4860 | TGAGAACTGGATGCT | A574858Hi | +T*+G*+A*G*A*A*C*T*G*G*A*T*G*C*T*C*+ | 135528 | 19 |
| | CTTG | | T*+T*+G | | |
| 4861 | GCATCAGATTGATGT | A574859Hi | +G*+C*+A*T*C*A*G*A*T*T*G*A*T*G*T*+A* | 135609 | 18 |
| | ACA | | +C*+A | | |
| 4862 | TGCATCAGATTGATG | A574860Hi | +T*+G*+C*A*T*C*A*G*A*T*T*G*A*T*G*+T* | 135610 | 18 |
| | TAC | | +A*+C | | |
| 4863 | ATTGCATCAGATTGA | A574861Hi | +A*+T*+T*G*C*A*T*C*A*G*A*T*T*G*A*+T* | 135612 | 18 |
| | TGT | | +G*+T | | |
| 4864 | GCAAGCACTGAATTA | A574862Hi | +G*+C*+A*A*G*C*A*C*T*G*A*A*T*T*A*T*+ | 135710 | 19 |
| | TGAA | | G*+A*+A | | |
| 4865 | GCAAGCACTGAATTA | A574863Hi | +G*+C*+A*A*G*C*A*C*T*G*A*A*T*T*A*+T* | 135711 | 18 |
| | TGA | | +G*+A | | |
| 4866 | CTGAGTCAATTTCAG | A574864Hi | +C*+T*+G*A*G*T*C*A*A*T*T*T*C*A*G*T*+ | 135780 | 19 |
| | TTTG | | T*+T*+G | | |
| 4867 | GATCTGAGTCAATTT | A574865Hi | +G*+A*+T*C*T*G*A*G*T*C*A*A*T*T*T*C*+ | 135783 | 19 |
| | CAGT | | A*+G*+T | | |
| 4868 | AGATCTGAGTCAATT | A574866Hi | +A*+G*+A*T*C*T*G*A*G*T*C*A*A*T*T*T*+ | 135784 | 19 |
| | TCAG | | C*+A*+G | | |
| 4869 | GAGATCTGAGTCAAT | A574867Hi | +G*+A*+G*A*T*C*T*G*A*G*T*C*A*A*T*T*+ | 135785 | 19 |
| | TTCA | | T*+C*+A | | |
| 4870 | CTTAGAGATCTGAGT | A574868Hi | +C*+T*+T*A*G*A*G*A*T*C*T*G*A*G*T*C*+ | 135789 | 19 |
| | CAAT | | A*+A*+T | | |
| 4871 | CTGCCACTCAGAGCA | A574869Hi | +C*+T*+G*C*C*A*C*T*C*A*G*A*G*C*A*A*+ | 135903 | 19 |
| | AGTG | | G*+T*+G | | |
| 4872 | CTAACCTGCCACTCA | A574870Hi | +C*+T*+A*A*C*C*T*G*C*C*A*C*T*C*A*G*+ | 135908 | 19 |
| | GAGC | | A*+G*+C | | |
| 4873 | CTAACCTGCCACTCA | A574871Hi | +C*+T*+A*A*C*C*T*G*C*C*A*C*T*C*A*+G* | 135909 | 18 |
| | GAG | | +A*+G | | |
| 4874 | CCTAACCTGCCACTC | A574872Hi | +C*+C*+T*A*A*C*C*T*G*C*C*A*C*T*C*A*+ | 135909 | 19 |
| | AGAG | | G*+A*+G | | |
| 4875 | AGCTACCTAGTGGTT | A574873Hi | +A*+G*+C*T*A*C*C*T*A*G*T*G*G*T*T*T*+ | 136044 | 19 |
| | TCTA | | C*+T*+A | | |
| 4876 | GCTACCTAGTGGTTT | A574874Hi | +G*+C*+T*A*C*C*T*A*G*T*G*G*T*T*T*+C* | 136044 | 18 |
| | CTA | | +T*+A | | |
| 4877 | GCAGTATATCTTAAC | A574875Hi | +G*+C*+A*G*T*A*T*A*T*C*T*T*A*A*C*+C* | 136177 | 18 |
| | CTC | | +T*+C | | |
| 4878 | GCAGTATATCTTAAC | A574876Hi | +G*+C*+A*G*T*A*T*A*T*C*T*T*A*A*+C*+C | 136178 | 17 |
| | CT | | *+T | | |
| 4879 | ATGTTAACCACATAC | A574877Hi | +A*+T*+G*T*T*A*A*C*C*A*C*A*T*A*C*T*+ | 136260 | 19 |
| | TAGT | | A*+G*+T | | |
| 4880 | TGTTAACCACATACT | A574878Hi | +T*+G*+T*T*A*A*C*C*A*C*A*T*A*C*T*+A* | 136260 | 18 |
| | AGT | | +G*+T | | |
| 4881 | AATGTTAACCACATA | A574879Hi | +A*+A*+T*G*T*T*A*A*C*C*A*C*A*T*A*C*+ | 136261 | 19 |
| | CTAG | | T*+A*+G | | |
| 4882 | CTCTCTAGTAAAGCA | A574880Hi | +C*+T*+C*T*C*T*A*G*T*A*A*A*G*C*A*+T* | 136655 | 18 |
| | TCT | | +C*+T | | |
| 4883 | GCTCTCTAGTAAAGC | A574881Hi | +G*+C*+T*C*T*C*T*A*G*T*A*A*A*G*C*+A* | 136656 | 18 |
| | ATC | | +T*+C | | |
| 4884 | GATGCTCTCTAGTAA | A574882Hi | +G*+A*+T*G*C*T*C*T*C*T*A*G*T*A*A*+A* | 136659 | 18 |
| | AGC | | +G*+C | | |
| 4885 | TATGGATGCTCTCTA | A574883Hi | +T*+A*+T*G*G*A*T*G*C*T*C*T*C*T*A*+G* | 136663 | 18 |
| | GTA | | +T*+A | | |
| 4886 | ATCTTTATGGATGCT | A574884Hi | +A*+T*+C*T*T*T*A*T*G*G*A*T*G*C*T*C*+ | 136667 | 19 |
| | CTCT | | T*+C*+T | | |
| 4887 | CTTGATCTTTATGGA | A574885Hi | +C*+T*+T*G*A*T*C*T*T*T*A*T*G*G*A*+T* | 136672 | 18 |
| | TGC | | +G*+C | | |
| 4888 | GAGAGCTTGATCTTT | A574886Hi | +G*+A*+G*A*G*C*T*T*G*A*T*C*T*T*T*A*+ | 136676 | 19 |
| | ATGG | | T*+G*+G | | |
| 4889 | TGAGAGCTTGATCTT | A574887Hi | +T*+G*+A*G*A*G*C*T*T*G*A*T*C*T*T*+T* | 136678 | 18 |
| | TAT | | +A*+T | | |
| 4890 | ATCTTTGAGAGCTTG | A574888Hi | +A*+T*+C*T*T*T*G*A*G*A*G*C*T*T*G*A*+ | 136682 | 19 |
| | ATCT | | T*+C*+T | | |
| 4891 | ATCTTTGAGAGCTTG | A574889Hi | +A*+T*+C*T*T*T*G*A*G*A*G*C*T*T*G*+A* | 136683 | 18 |
| | ATC | | +T*+C | | |
| 4892 | TCAGAAGAGAGCAAA | A574890Hi | +T*+C*+A*G*A*A*G*A*G*A*G*C*A*A*A*T*+ | 136752 | 19 |
| | TATT | | A*+T*+T | | |
| 4893 | ACTTGTTCAGAAGAG | A574891Hi | +A*+C*+T*T*G*T*T*C*A*G*A*A*G*A*G*+A* | 136759 | 18 |
| | AGC | | +G*+C | | |
| 4894 | GATACTTGTTCAGAA | A574892Hi | +G*+A*+T*A*C*T*T*G*T*T*C*A*G*A*A*G*+ | 136761 | 19 |
| | GAGA | | A*+G*+A | | |
| 4895 | CACAGGAGATACTTG | A574893Hi | +C*+A*+C*A*G*G*A*G*A*T*A*C*T*T*G*T*+ | 136768 | 19 |
| | TTCA | | T*+C*+A | | |
| 4896 | AGGCCACAGGAGATA | A574894Hi | +A*+G*+G*C*C*A*C*A*G*G*A*G*A*T*A*C* | 136772 | 19 |
| | CTTG | | +T*+T*+G | | |
| 4897 | TGCCTCTCAGTCGGT | A574895Hi | +T*+G*+C*C*T*C*T*C*A*G*T*C*G*G*T*G*+ | 136869 | 19 |
| | GTGT | | T*+G*+T | | |
| 4898 | GCCTCTCAGTCGGTG | A574896Hi | +G*+C*+C*T*C*T*C*A*G*T*C*G*G*T*G*+T* | 136869 | 18 |
| | TGT | | +G*+T | | |
| 4899 | TGCCTCTCAGTCGGT | A574897Hi | +T*+G*+C*C*T*C*T*C*A*G*T*C*G*G*T*+G* | 136870 | 18 |
| | GTG | | +T*+G | | |
| 4900 | GCCTCTCAGTCGGTG | A574898Hi | +G*+C*+C*T*C*T*C*A*G*T*C*G*G*T*+G*+T | 136870 | 17 |
| | TG | | *+G | | |
| 4901 | ATGCCTCTCAGTCGG | A574899Hi | +A*+T*+G*C*C*T*C*T*C*A*G*T*C*G*G*+T* | 136871 | 18 |
| | TGT | | +G*+T | | |
| 4902 | GCAGTCATCTTTCAT | A574900Hi | +G*+C*+A*G*T*C*A*T*C*T*T*T*C*A*T*+T* | 136894 | 18 |
| | TAC | | +A*+C | | |
| 4903 | GGCAGTCATCTTTCA | A574901Hi | +G*+G*+C*A*G*T*C*A*T*C*T*T*T*C*A*T*+ | 136894 | 19 |
| | TTAC | | T*+A*+C | | |
| 4904 | TGCAGACCTAGAACT | A574902Hi | +T*+G*+C*A*G*A*C*C*T*A*G*A*A*C*T*+C* | 136942 | 18 |
| | CCA | | +C*+A | | |
| 4905 | TGTTGCAGACCTAGA | A574903Hi | +T*+G*+T*T*G*C*A*G*A*C*C*T*A*G*A*A*+ | 136944 | 19 |
| | ACTC | | C*+T*+C | | |
| 4906 | AACATGTTGCAGACC | A574904Hi | +A*+A*+C*A*T*G*T*T*G*C*A*G*A*C*C*+T* | 136949 | 18 |
| | TAG | | +A*+G | | |
| 4907 | ATGATGTGTTGTCAG | A574905Hi | +A*+T*+G*A*T*G*T*G*T*T*G*T*C*A*G*A*+ | 137224 | 19 |
| | AGAG | | G*+A*+G | | |
| 4908 | ATATGATGTGTTGTC | A574906Hi | +A*+T*+A*T*G*A*T*G*T*G*T*T*G*T*C*A*+ | 137226 | 19 |
| | AGAG | | G*+A*+G | | |
| 4909 | TATGATGTGTTGTCA | A574907Hi | +T*+A*+T*G*A*T*G*T*G*T*T*G*T*C*A*+G* | 137226 | 18 |
| | GAG | | +A*+G | | |
| 4910 | AATATGATGTGTTGT | A574908Hi | +A*+A*+T*A*T*G*A*T*G*T*G*T*T*G*T*C*+ | 137227 | 19 |
| | CAGA | | A*+G*+A | | |
| 4911 | TAAATCAGATAGATA | A574909Hi | +T*+A*+A*A*T*C*A*G*A*T*A*G*A*T*A*A*+ | 137343 | 19 |
| | ACAT | | C*+A*+T | | |
| 4912 | TAAATCAGATAGATA | A574910Hi | +T*+A*+A*A*T*C*A*G*A*T*A*G*A*T*A*+A* | 137344 | 18 |
| | ACA | | +C*+A | | |
| 4913 | TTAAATCAGATAGAT | A574911Hi | +T*+T*+A*A*A*T*C*A*G*A*T*A*G*A*T*A*+ | 137344 | 19 |
| | AACA | | A*+C*+A | | |
| 4914 | TTTAAATCAGATAGA | A574912Hi | +T*+T*+T*A*A*A*T*C*A*G*A*T*A*G*A*T*+ | 137345 | 19 |
| | TAAC | | A*+A*+C | | |
| 4915 | GCATTTAAATCAGAT | A574913Hi | +G*+C*+A*T*T*T*A*A*A*T*C*A*G*A*T*A*+ | 137348 | 19 |
| | AGAT | | G*+A*+T | | |
| 4916 | GTCACTGTCATATTA | A574914Hi | +G*+T*+C*A*C*T*G*T*C*A*T*A*T*T*A*T*+ | 137592 | 19 |
| | TAGA | | A*+G*+A | | |
| 4917 | TGTCACTGTCATATT | A574915Hi | +T*+G*+T*C*A*C*T*G*T*C*A*T*A*T*T*A*+ | 137593 | 19 |
| | ATAG | | T*+A*+G | | |
| 4918 | TGGTGTCACTGTCAT | A574916Hi | +T*+G*+G*T*G*T*C*A*C*T*G*T*C*A*T*A*+ | 137596 | 19 |
| | ATTA | | T*+T*+A | | |
| 4919 | TCACAAACAGATTGG | A574917Hi | +T*+C*+A*C*A*A*A*C*A*G*A*T*T*G*G*C*+ | 137734 | 19 |
| | CTAC | | T*+A*+C | | |
| 4920 | TTTCACAAACAGATT | A574918Hi | +T*+T*+T*C*A*C*A*A*A*C*A*G*A*T*T*G*+ | 137736 | 19 |
| | GGCT | | G*+C*+T | | |
| 4921 | GTACATATTTCACAA | A574919Hi | +G*+T*+A*C*A*T*A*T*T*T*C*A*C*A*A*A*+ | 137743 | 19 |
| | ACAG | | C*+A*+G | | |
| 4922 | TAGTTGCAGTGTCTT | A574920Hi | +T*+A*+G*T*T*G*C*A*G*T*G*T*C*T*T*C*+ | 137936 | 19 |
| | CAAA | | A*+A*+A | | |
| 4923 | TTAGTTGCAGTGTCT | A574921Hi | +T*+T*+A*G*T*T*G*C*A*G*T*G*T*C*T*T*+ | 137937 | 19 |
| | TCAA | | C*+A*+A | | |
| 4924 | TTTAGTTGCAGTGTC | A574922Hi | +T*+T*+T*A*G*T*T*G*C*A*G*T*G*T*C*T*+ | 137938 | 19 |
| | TTCA | | T*+C*+A | | |
| 4925 | ATTTAGTTGCAGTGT | A574923Hi | +A*+T*+T*T*A*G*T*T*G*C*A*G*T*G*T*C*+ | 137939 | 19 |
| | CTTC | | T*+T*+C | | |
| 4926 | AAAGATTATGTCCAA | A574924Hi | +A*+A*+A*G*A*T*T*A*T*G*T*C*C*A*A*T*+ | 138151 | 19 |
| | TGTT | | G*+T*+T | | |
| 4927 | TATATAACATGCTTT | A574925Hi | +T*+A*+T*A*T*A*A*C*A*T*G*C*T*T*T*G*+ | 138219 | 19 |
| | GGTA | | G*+T*+A | | |
| 4928 | ATATATAACATGCTTT | A574926Hi | +A*+T*+A*T*A*T*A*A*C*A*T*G*C*T*T*T*+ | 138220 | 19 |
| | GGT | | G*+G*+T | | |
| 4929 | TATATAACATGCTTT | A574927Hi | +T*+A*+T*A*T*A*A*C*A*T*G*C*T*T*T*+G* | 138220 | 18 |
| | GGT | | +G*+T | | |
| 4930 | GCTAGACTATTGCAA | A574928Hi | +G*+C*+T*A*G*A*C*T*A*T*T*G*C*A*A*T*+ | 138270 | 19 |
| | TTAT | | T*+A*+T | | |
| 4931 | CTAGACTATTGCAAT | A574929Hi | +C*+T*+A*G*A*C*T*A*T*T*G*C*A*A*T*+T* | 138270 | 18 |
| | TAT | | +A*+T | | |
| 4932 | AGCTAGACTATTGCA | A574930Hi | +A*+G*+C*T*A*G*A*C*T*A*T*T*G*C*A*A*+ | 138271 | 19 |
| | ATTA | | T*+T*+A | | |
| 4933 | GCTAGACTATTGCAA | A574931Hi | +G*+C*+T*A*G*A*C*T*A*T*T*G*C*A*A*+T* | 138271 | 18 |
| | TTA | | +T*+A | | |
| 4934 | AAACAAGCTAGACTA | A574932Hi | +A*+A*+A*C*A*A*G*C*T*A*G*A*C*T*A*T*+ | 138276 | 19 |
| | TTGC | | T*+G*+C | | |
| 4935 | TAAACAAGCTAGACT | A574933Hi | +T*+A*+A*A*C*A*A*G*C*T*A*G*A*C*T*A*+ | 138277 | 19 |
| | ATTG | | T*+T*+G | | |
| 4936 | CTAAACAAGCTAGAC | A574934Hi | +C*+T*+A*A*A*C*A*A*G*C*T*A*G*A*C*T*+ | 138278 | 19 |
| | TATT | | A*+T*+T | | |
| 4937 | CTAAACAAGCTAGAC | A574935Hi | +C*+T*+A*A*A*C*A*A*G*C*T*A*G*A*C*+T* | 138279 | 18 |
| | TAT | | +A*+T | | |
| 4938 | CTAAACAAGCTAGAC | A574936Hi | +C*+T*+A*A*A*C*A*A*G*C*T*A*G*A*+C*+T | 138280 | 17 |
| | TA | | *+A | | |
| 4939 | ACTAAACAAGCTAGA | A574937Hi | +A*+C*+T*A*A*A*C*A*A*G*C*T*A*G*A*+C* | 138280 | 18 |
| | CTA | | +T*+A | | |
| 4940 | AACTAAACAAGCTAG | A574938Hi | +A*+A*+C*T*A*A*A*C*A*A*G*C*T*A*G*+A* | 138281 | 18 |
| | ACT | | +C*+T | | |
| 4941 | AAATTTCTACGTGTA | A574939Hi | +A*+A*+A*T*T*T*C*T*A*C*G*T*G*T*A*A*+ | 138339 | 19 |
| | ATAC | | T*+A*+C | | |
| 4942 | AATTTCTACGTGTAA | A574940Hi | +A*+A*+T*T*T*C*T*A*C*G*T*G*T*A*A*+T* | 138339 | 18 |
| | TAC | | +A*+C | | |
| 4943 | AGTCATCACTATTTG | A574941Hi | +A*+G*+T*C*A*T*C*A*C*T*A*T*T*T*G*+T* | 138366 | 18 |
| | TTT | | +T*+T | | |
| 4944 | AAGTCATCACTATTT | A574942Hi | +A*+A*+G*T*C*A*T*C*A*C*T*A*T*T*T*G*+ | 138366 | 19 |
| | GTTT | | T*+T*+T | | |
| 4945 | GTTGGAATCACTGAA | A574943H | +G*+T*+T*G*G*A*A*T*C*A*C*T*G*A*A*A*+ | 138418 | 19 |
| | AGCT | | G*+C*+T | | |
| 4946 | TGTTGGAATCACTGA | A574944H | +T*+G*+T*T*G*G*A*A*T*C*A*C*T*G*A*A*+ | 138419 | 19 |
| | AAGC | | A*+G*+C | | |
| 4947 | GTGTCTACCACCACT | A574945H | +G*+T*+G*T*C*T*A*C*C*A*C*C*A*C*T*G*+ | 138441 | 19 |
| | GTTA | | T*+T*+A | | |
| 4948 | GTGTCTACCACCACT | A574946H | +G*+T*+G*T*C*T*A*C*C*A*C*C*A*C*T*+G* | 138442 | 18 |
| | GTT | | +T*+T | | |
| 4949 | AGTGTCTACCACCAC | A574947H | +A*+G*+T*G*T*C*T*A*C*C*A*C*C*A*C*T*+ | 138442 | 19 |
| | TGTT | | G*+T*+T | | |
| 4950 | TCCACAACAGGGCTA | A574948H | +T*+C*+C*A*C*A*A*C*A*G*G*G*C*T*A*T*+ | 138480 | 19 |
| | TTTT | | T*+T*+T | | |
| 4951 | CTTCCACAACAGGGC | A574949H | +C*+T*+T*C*C*A*C*A*A*C*A*G*G*G*C*T*+ | 138482 | 19 |
| | TATT | | A*+T*+T | | |
| 4952 | ACTTCCACAACAGGG | A574950H | +A*+C*+T*T*C*C*A*C*A*A*C*A*G*G*G*C*+ | 138483 | 19 |
| | CTAT | | T*+A*+T | | |
| 4953 | ACTTCCACAACAGGG | A574951H | +A*+C*+T*T*C*C*A*C*A*A*C*A*G*G*G*+C* | 138484 | 18 |
| | CTA | | +T*+A | | |
| 4954 | CACTTCCACAACAGG | A574952H | +C*+A*+C*T*T*C*C*A*C*A*A*C*A*G*G*G*+ | 138484 | 19 |
| | GCTA | | C*+T*+A | | |
| 4955 | CCCACACTTCCACAA | A574953H | +C*+C*+C*A*C*A*C*T*T*C*C*A*C*A*A*C*+ | 138488 | 19 |
| | CAGG | | A*+G*+G | | |
| 4956 | TCCCACACTTCCACA | A574954H | +T*+C*+C*C*A*C*A*C*T*T*C*C*A*C*A*A*+ | 138489 | 19 |
| | ACAG | | C*+A*+G | | |
| 4957 | TATCCCACACTTCCA | A574955H | +T*+A*+T*C*C*C*A*C*A*C*T*T*C*C*A*C*+ | 138491 | 19 |
| | CAAC | | A*+A*+C | | |
| 4958 | TATCCCACACTTCCA | A574956H | +T*+A*+T*C*C*C*A*C*A*C*T*T*C*C*A*+C* | 138492 | 18 |
| | CAA | | +A*+A | | |
| 4959 | TTATCCCACACTTCC | A574957H | +T*+T*+A*T*C*C*C*A*C*A*C*T*T*C*C*A*+ | 138492 | 19 |
| | ACAA | | C*+A*+A | | |
| 4960 | TCTTATCCCACACTTC | A574958H | +T*+C*+T*T*A*T*C*C*C*A*C*A*C*T*T*C*+ | 138494 | 19 |
| | CAC | | C*+A*+C | | |
| 4961 | GTTTTCTTATCCCACA | A574959H | +G*+T*+T*T*T*C*T*T*A*T*C*C*C*A*C*A*+ | 138498 | 19 |
| | CTT | | C*+T*+T | | |
| 4962 | AGTTTTCTTATCCCAC | A574960H | +A*+G*+T*T*T*T*C*T*T*A*T*C*C*C*A*C*+ | 138499 | 19 |
| | ACT | | A*+C*+T | | |
| 4963 | CATGAAGTGCAAAGA | A574961Hi | +C*+A*+T*G*A*A*G*T*G*C*A*A*A*G*A*T*+ | 138595 | 19 |
| | TAAT | | A*+A*+T | | |
| 4964 | CACATGAAGTGCAAA | A574962Hi | +C*+A*+C*A*T*G*A*A*G*T*G*C*A*A*A*G*+ | 138597 | 19 |
| | GATA | | A*+T*+A | | |
| 4965 | GGTGCTGAGAAGCAT | A574963Hi | +G*+G*+T*G*C*T*G*A*G*A*A*G*C*A*T*T*+ | 138686 | 19 |
| | TACA | | A*+C*+A | | |
| 4966 | TGGTGCTGAGAAGCA | A574964Hi | +T*+G*+G*T*G*C*T*G*A*G*A*A*G*C*A*T*+ | 138687 | 19 |
| | TTAC | | T*+A*+C | | |
| 4967 | TGGTGCTGAGAAGCA | A574965Hi | +T*+G*+G*T*G*C*T*G*A*G*A*A*G*C*A*+T* | 138688 | 18 |
| | TTA | | +T*+A | | |
| 4968 | TATATTTGACACTGT | A574966Hi | +T*+A*+T*A*T*T*T*G*A*C*A*C*T*G*T*+G* | 138864 | 18 |
| | GAC | | +A*+C | | |
| 4969 | GACTAAGCACTATGC | A574967Hi | +G*+A*+C*T*A*A*G*C*A*C*T*A*T*G*C*+T* | 139017 | 18 |
| | TCT | | +C*+T | | |
| 4970 | GACTAAGCACTATGC | A574968Hi | +G*+A*+C*T*A*A*G*C*A*C*T*A*T*G*+C*+T | 139018 | 17 |
| | TC | | *+C | | |
| 4971 | TCAGACTAAGCACTA | A574969Hi | +T*+C*+A*G*A*C*T*A*A*G*C*A*C*T*A*T*+ | 139019 | 19 |
| | TGCT | | G*+C*+T | | |
| 4972 | CATCAGACTAAGCAC | A574970Hi | +C*+A*+T*C*A*G*A*C*T*A*A*G*C*A*C*T*+ | 139021 | 19 |
| | TATG | | A*+T*+G | | |
| 4973 | GCATCAGACTAAGCA | A574971Hi | +G*+C*+A*T*C*A*G*A*C*T*A*A*G*C*A*+C* | 139023 | 18 |
| | CTA | | +T*+A | | |
| 4974 | GGCTGCATCAGACTA | A574972Hi | +G*+G*+C*T*G*C*A*T*C*A*G*A*C*T*A*A*+ | 139026 | 19 |
| | AGCA | | G*+C*+A | | |
| 4975 | TGTGCTTGCTATGGA | A574973Hi | +T*+G*+T*G*C*T*T*G*C*T*A*T*G*G*A*T*+ | 139054 | 19 |
| | TTTG | | T*+T*+G | | |
| 4976 | AATGTGCTTGCTATG | A574974Hi | +A*+A*+T*G*T*G*C*T*T*G*C*T*A*T*G*+G* | 139057 | 18 |
| | GAT | | +A*+T | | |
| 4977 | GAATGTGCTTGCTAT | A574975Hi | +G*+A*+A*T*G*T*G*C*T*T*G*C*T*A*T*+G* | 139058 | 18 |
| | GGA | | +G*+A | | |
| 4978 | GCACAGAAAGAATGT | A574976Hi | +G*+C*+A*C*A*G*A*A*A*G*A*A*T*G*T*G*+ | 139066 | 19 |
| | GCTT | | C*+T*+T | | |
| 4979 | ACCAGCACAGAAAGA | A574977Hi | +A*+C*+C*A*G*C*A*C*A*G*A*A*A*G*A*A*+ | 139070 | 19 |
| | ATGT | | T*+G*+T | | |
| 4980 | GCTAGCAAATAAGCT | A574978Hi | +G*+C*+T*A*G*C*A*A*A*T*A*A*G*C*T*T*+ | 139108 | 19 |
| | TGAA | | G*+A*+A | | |
| 4981 | TGGCTAGCAAATAAG | A574979Hi | +T*+G*+G*C*T*A*G*C*A*A*A*T*A*A*G*+C* | 139111 | 18 |
| | CTT | | +T*+T | | |
| 4982 | AAGTGGCTAGCAAAT | A574980Hi | +A*+A*+G*T*G*G*C*T*A*G*C*A*A*A*T*A*+ | 139113 | 19 |
| | AAGC | | A*+G*+C | | |
| 4983 | ATCAAGGAGACCTGT | A574981Hi | +A*+T*+C*A*A*G*G*A*G*A*C*C*T*G*T*+T* | 139302 | 18 |
| | TGG | | +G*+G | | |
| 4984 | CCTTTATCAAGGAGA | A574982Hi | +C*+C*+T*T*T*A*T*C*A*A*G*G*A*G*A*C*+ | 139306 | 19 |
| | CCTG | | C*+T*+G | | |
| 4985 | AAAGAATCCTAATCG | A574983Hi | +A*+A*+A*G*A*A*T*C*C*T*A*A*T*C*G*+T* | 139348 | 18 |
| | TTT | | +T*+T | | |
| 4986 | CAGAACCAGTGAATA | A574984Hi | +C*+A*+G*A*A*C*C*A*G*T*G*A*A*T*A*G*+ | 139398 | 19 |
| | GTCT | | T*+C*+T | | |
| 4987 | CAGAACCAGTGAATA | A574985Hi | +C*+A*+G*A*A*C*C*A*G*T*G*A*A*T*A*+G* | 139399 | 18 |
| | GTC | | +T*+C | | |
| 4988 | CCAGAACCAGTGAAT | A574986Hi | +C*+C*+A*G*A*A*C*C*A*G*T*G*A*A*T*+A* | 139400 | 18 |
| | AGT | | +G*+T | | |
| 4989 | GGAAATTATGAATAA | A574987Hi | +G*+G*+A*A*A*T*T*A*T*G*A*A*T*A*A*G*+ | 139600 | 19 |
| | GAGG | | A*+G*+G | | |
| 4990 | CTTTACTTGAAGCAT | A574988Hi | +C*+T*+T*T*A*C*T*T*G*A*A*G*C*A*T*A*+ | 139650 | 19 |
| | AAAT | | A*+A*+T | | |
| 4991 | TTTGTGTTTTGATTCC | A574989H | +T*+T*+T*G*T*G*T*T*T*T*G*A*T*T*C*C*+ | 139867 | 19 |
| | TTG | | T*+T*+G | | |
| 4992 | AGACATTTTGTGTTTT | A574990H | +A*+G*+A*C*A*T*T*T*T*G*T*G*T*T*T*T*+ | 139873 | 19 |
| | GAT | | G*+A*+T | | |
| 4993 | GAATAAGACATTTTG | A574991H | +G*+A*+A*T*A*A*G*A*C*A*T*T*T*T*G*T*+ | 139878 | 19 |
| | TGTT | | G*+T*+T | | |
| 4994 | AGAATAAGACATTTT | A574992H | +A*+G*+A*A*T*A*A*G*A*C*A*T*T*T*T*G*+ | 139879 | 19 |
| | GTGT | | T*+G*+T | | |
| 4995 | CCAGAATAAGACATT | A574993H | +C*+C*+A*G*A*A*T*A*A*G*A*C*A*T*T*T*+ | 139881 | 19 |
| | TTGT | | T*+G*+T | | |
| 4996 | CTCTCCCAGAATAAG | A574994H | +C*+T*+C*T*C*C*C*A*G*A*A*T*A*A*G*A*+ | 139886 | 19 |
| | ACAT | | C*+A*+T | | |
| 4997 | TTTCACTCTCCCAGA | A574995H | +T*+T*+T*C*A*C*T*C*T*C*C*C*A*G*A*A*+ | 139891 | 19 |
| | ATAA | | T*+A*+A | | |
| 4998 | GTGTTCTTCTGAAGG | A574996H | +G*+T*+G*T*T*C*T*T*C*T*G*A*A*G*G*C*+ | 139914 | 19 |
| | CAGA | | A*+G*+A | | |
| 4999 | GTGTTCTTCTGAAGG | A574997H | +G*+T*+G*T*T*C*T*T*C*T*G*A*A*G*G*+C* | 139915 | 18 |
| | CAG | | +A*+G | | |
| 5000 | AGTGTTCTTCTGAAG | A574998H | +A*+G*+T*G*T*T*C*T*T*C*T*G*A*A*G*G*+ | 139915 | 19 |
| | GCAG | | C*+A*+G | | |
| 5001 | AGAGCAGTGTTCTTC | A574999H | +A*+G*+A*G*C*A*G*T*G*T*T*C*T*T*C*T*+ | 139920 | 19 |
| | TGAA | | G*+A*+A | | |
| 5002 | AAGAGCAGTGTTCTT | A575000H | +A*+A*+G*A*G*C*A*G*T*G*T*T*C*T*T*C*+ | 139921 | 19 |
| | CTGA | | T*+G*+A | | |
| 5003 | AAAGAGCAGTGTTCT | A575001H | +A*+A*+A*G*A*G*C*A*G*T*G*T*T*C*T*T*+ | 139922 | 19 |
| | TCTG | | C*+T*+G | | |
| 5004 | CCAAAGAGCAGTGTT | A575002H | +C*+C*+A*A*A*G*A*G*C*A*G*T*G*T*T*C*+ | 139924 | 19 |
| | CTTC | | T*+T*+C | | |
| 5005 | TATCCAAAGAGCAGT | A575003H | +T*+A*+T*C*C*A*A*A*G*A*G*C*A*G*T*G*+ | 139927 | 19 |
| | GTTC | | T*+T*+C | | |
| 5006 | CTATCCAAAGAGCAG | A575004H | +C*+T*+A*T*C*C*A*A*A*G*A*G*C*A*G*T*+ | 139928 | 19 |
| | TGTT | | G*+T*+T | | |
| 5007 | TTCCTATCCAAAGAG | A575005H | +T*+T*+C*C*T*A*T*C*C*A*A*A*G*A*G*C*+ | 139931 | 19 |
| | CAGT | | A*+G*+T | | |
| 5008 | GTTCCTATCCAAAGA | A575006H | +G*+T*+T*C*C*T*A*T*C*C*A*A*A*G*A*G*+ | 139932 | 19 |
| | GCAG | | C*+A*+G | | |
| 5009 | TCCAGTTCCTATCCA | A575007H | +T*+C*+C*A*G*T*T*C*C*T*A*T*C*C*A*A*+ | 139936 | 19 |
| | AAGA | | A*+G*+A | | |
| 5010 | CTCCAGTTCCTATCC | A575008H | +C*+T*+C*C*A*G*T*T*C*C*T*A*T*C*C*A*+ | 139937 | 19 |
| | AAAG | | A*+A*+G | | |
| 5011 | TCCTCCAGTTCCTAT | A575009H | +T*+C*+C*T*C*C*A*G*T*T*C*C*T*A*T*C*+ | 139939 | 19 |
| | CCAA | | C*+A*+A | | |
| 5012 | GCCTCCTCCAGTTCC | A575010H | +G*+C*+C*T*C*C*T*C*C*A*G*T*T*C*C*T*+ | 139942 | 19 |
| | TATC | | A*+T*+C | | |
| 5013 | GGCCTCCTCCAGTTC | A575011H | +G*+G*+C*C*T*C*C*T*C*C*A*G*T*T*C*C*+ | 139943 | 19 |
| | CTAT | | T*+A*+T | | |
| 5014 | GGCCTCCTCCAGTTC | A575012H | +G*+G*+C*C*T*C*C*T*C*C*A*G*T*T*C*+C* | 139944 | 18 |
| | CTA | | +T*+A | | |
| 5015 | TGGCCTCCTCCAGTT | A575013H | +T*+G*+G*C*C*T*C*C*T*C*C*A*G*T*T*C*+ | 139944 | 19 |
| | CCTA | | C*+T*+A | | |
| 5016 | AATATGGCCTCCTCC | A575014H | +A*+A*+T*A*T*G*G*C*C*T*C*C*T*C*C*A*+ | 139948 | 19 |
| | AGTT | | G*+T*+T | | |
| 5017 | AAATATGGCCTCCTC | A575015H | +A*+A*+A*T*A*T*G*G*C*C*T*C*C*T*C*C*+ | 139949 | 19 |
| | CAGT | | A*+G*+T | | |
| 5018 | TAAAATATGGCCTCC | A575016H | +T*+A*+A*A*A*T*A*T*G*G*C*C*T*C*C*T*+ | 139951 | 19 |
| | TCCA | | C*+C*+A | | |
| 5019 | GTAAAATATGGCCTC | A575017H | +G*+T*+A*A*A*A*T*A*T*G*G*C*C*T*C*C*+ | 139952 | 19 |
| | CTCC | | T*+C*+C | | |
| 5020 | GTAAAATATGGCCTC | A575018H | +G*+T*+A*A*A*A*T*A*T*G*G*C*C*T*C*+C* | 139953 | 18 |
| | CTC | | +T*+C | | |
| 5021 | AGTAAAATATGGCCT | A575019H | +A*+G*+T*A*A*A*A*T*A*T*G*G*C*C*T*C*+ | 139953 | 19 |
| | CCTC | | C*+T*+C | | |
| 5022 | GAGTAAAATATGGCC | A575020H | +G*+A*+G*T*A*A*A*A*T*A*T*G*G*C*C*T*+ | 139954 | 19 |
| | TCCT | | C*+C*+T | | |
| 5023 | GACGAGTTGAAAGAT | A575021H | +G*+A*+C*G*A*G*T*T*G*A*A*A*G*A*T*+C* | 139974 | 18 |
| | CCA | | +C*+A | | |
| 5024 | CGACGAGTTGAAAGA | A575022H | +C*+G*+A*C*G*A*G*T*T*G*A*A*A*G*A*T*+ | 139974 | 19 |
| | TCCA | | C*+C*+A | | |
| 5025 | GACGAGTTGAAAGAT | A575023H | +G*+A*+C*G*A*G*T*T*G*A*A*A*G*A*+T*+ | 139975 | 17 |
| | CC | | C*+C | | |
| 5026 | CGACGAGTTGAAAGA | A575024H | +C*+G*+A*C*G*A*G*T*T*G*A*A*A*G*A*+T* | 139975 | 18 |
| | TCC | | +C*+C | | |
| 5027 | TCGACGAGTTGAAAG | A575025H | +T*+C*+G*A*C*G*A*G*T*T*G*A*A*A*G*A*+ | 139975 | 19 |
| | ATCC | | T*+C*+C | | |
| 5028 | CGACGAGTTGAAAGA | A575026H | +C*+G*+A*C*G*A*G*T*T*G*A*A*A*G*+A*+ | 139976 | 17 |
| | TC | | T*+C | | |
| 5029 | TCGACGAGTTGAAAG | A575027H | +T*+C*+G*A*C*G*A*G*T*T*G*A*A*A*G*+A* | 139976 | 18 |
| | ATC | | +T*+C | | |
| 5030 | TCATGACTCTGACCG | A575028H | +T*+C*+A*T*G*A*C*T*C*T*G*A*C*C*G*+A* | 140022 | 18 |
| | AAT | | +A*+T | | |
| 5031 | ATCATGACTCTGACC | A575029H | +A*+T*+C*A*T*G*A*C*T*C*T*G*A*C*C*G*+ | 140022 | 19 |
| | GAAT | | A*+A*+T | | |
| 5032 | TCATGACTCTGACCG | A575030H | +T*+C*+A*T*G*A*C*T*C*T*G*A*C*C*+G*+A | 140023 | 17 |
| | AA | | *+A | | |
| 5033 | ATCATGACTCTGACC | A575031H | +A*+T*+C*A*T*G*A*C*T*C*T*G*A*C*C*+G* | 140023 | 18 |
| | GAA | | +A*+A | | |
| 5034 | CATCATGACTCTGAC | A575032H | +C*+A*+T*C*A*T*G*A*C*T*C*T*G*A*C*C*+ | 140023 | 19 |
| | CGAA | | G*+A*+A | | |
| 5035 | CATCATGACTCTGAC | A575033H | +C*+A*+T*C*A*T*G*A*C*T*C*T*G*A*C*+C* | 140024 | 18 |
| | CGA | | +G*+A | | |
| 5036 | TCATCATGACTCTGA | A575034H | +T*+C*+A*T*C*A*T*G*A*C*T*C*T*G*A*C*+ | 140024 | 19 |
| | CCGA | | C*+G*+A | | |
| 5037 | TCATCATGACTCTGA | A575035H | +T*+C*+A*T*C*A*T*G*A*C*T*C*T*G*A*+C* | 140025 | 18 |
| | CCG | | +C*+G | | |
| 5038 | GTCATCATGACTCTG | A575036H | +G*+T*+C*A*T*C*A*T*G*A*C*T*C*T*G*A*+ | 140025 | 19 |
| | ACCG | | C*+C*+G | | |
| 5039 | TGTCATCATGACTCT | A575037H | +T*+G*+T*C*A*T*C*A*T*G*A*C*T*C*T*G*+ | 140026 | 19 |
| | GACC | | A*+C*+C | | |
| 5040 | GCTGTCATCATGACT | A575038H | +G*+C*+T*G*T*C*A*T*C*A*T*G*A*C*T*C*+ | 140028 | 19 |
| | CTGA | | T*+G*+A | | |
| 5041 | AGCTGTGCTGTCATC | A575039H | +A*+G*+C*T*G*T*G*C*T*G*T*C*A*T*C*A*+ | 140034 | 19 |
| | ATGA | | T*+G*+A | | |
| 5042 | CTAAAGGAAAGAAAC | A575040Hi | +C*+T*+A*A*A*G*G*A*A*A*G*A*A*A*C*T*+ | 140055 | 19 |
| | TTGC | | T*+G*+C | | |
| 5043 | TATAAGACTTAGAGA | A575041Hi | +T*+A*+T*A*A*G*A*C*T*T*A*G*A*G*A*A*+ | 140083 | 19 |
| | ATAT | | T*+A*+T | | |
| 5044 | TTATAAGACTTAGAG | A575042Hi | +T*+T*+A*T*A*A*G*A*C*T*T*A*G*A*G*A*+ | 140084 | 19 |
| | AATA | | A*+T*+A | | |
| 5045 | TTTATAAGACTTAGA | A575043Hi | +T*+T*+T*A*T*A*A*G*A*C*T*T*A*G*A*G*+ | 140085 | 19 |
| | GAAT | | A*+A*+T | | |
| 5046 | TGCAGTAATCTGCTT | A575044Hi | +T*+G*+C*A*G*T*A*A*T*C*T*G*C*T*T*C*+ | 140254 | 19 |
| | CTCT | | T*+C*+T | | |
| 5047 | GCAGTAATCTGCTTC | A575045Hi | +G*+C*+A*G*T*A*A*T*C*T*G*C*T*T*C*+T* | 140254 | 18 |
| | TCT | | +C*+T | | |
| 5048 | TGCAGTAATCTGCTT | A575046Hi | +T*+G*+C*A*G*T*A*A*T*C*T*G*C*T*T*+C* | 140255 | 18 |
| | CTC | | +T*+C | | |
| 5049 | AGCTGCAGTAATCTG | A575047Hi | +A*+G*+C*T*G*C*A*G*T*A*A*T*C*T*G*C*+ | 140257 | 19 |
| | CTTC | | T*+T*+C | | |
| 5050 | GCTGCAGTAATCTGC | A575048Hi | +G*+C*+T*G*C*A*G*T*A*A*T*C*T*G*C*+T* | 140257 | 18 |
| | TTC | | +T*+C | | |
| 5051 | TCAGCTGCAGTAATC | A575049Hi | +T*+C*+A*G*C*T*G*C*A*G*T*A*A*T*C*T*+ | 140259 | 19 |
| | TGCT | | G*+C*+T | | |
| 5052 | GTTCAGCTGCAGTAA | A575050Hi | +G*+T*+T*C*A*G*C*T*G*C*A*G*T*A*A*T*+ | 140261 | 19 |
| | TCTG | | C*+T*+G | | |
| 5053 | TGTTCAGCTGCAGTA | A575051Hi | +T*+G*+T*T*C*A*G*C*T*G*C*A*G*T*A*A*+ | 140262 | 19 |
| | ATCT | | T*+C*+T | | |
| 5054 | GTGTTCAGCTGCAGT | A575052Hi | +G*+T*+G*T*T*C*A*G*C*T*G*C*A*G*T*+A* | 140264 | 18 |
| | AAT | | +A*+T | | |
| 5055 | GGCTAGACTATGATT | A575053Hi | +G*+G*+C*T*A*G*A*C*T*A*T*G*A*T*T*+A* | 140393 | 18 |
| | AAA | | +A*+A | | |
| 5056 | GAGGCTAGACTATGA | A575054Hi | +G*+A*+G*G*C*T*A*G*A*C*T*A*T*G*A*T*+ | 140394 | 19 |
| | TTAA | | T*+A*+A | | |
| 5057 | GAGGCTAGACTATGA | A575055Hi | +G*+A*+G*G*C*T*A*G*A*C*T*A*T*G*A*+T* | 140395 | 18 |
| | TTA | | +T*+A | | |
| 5058 | ATGAGATTCTTTAAC | A575056Hi | +A*+T*+G*A*G*A*T*T*C*T*T*T*A*A*C*A*+ | 140416 | 19 |
| | AAGC | | A*+G*+C | | |
| 5059 | GCTTGTGTGGACATG | A575057Hi | +G*+C*+T*T*G*T*G*T*G*G*A*C*A*T*G*T*+ | 140901 | 19 |
| | TATC | | A*+T*+C | | |
| 5060 | CTGCTTGTGTGGACA | A575058Hi | +C*+T*+G*C*T*T*G*T*G*T*G*G*A*C*A*T*+ | 140903 | 19 |
| | TGTA | | G*+T*+A | | |
| 5061 | TCTGCTTGTGTGGAC | A575059Hi | +T*+C*+T*G*C*T*T*G*T*G*T*G*G*A*C*A*+ | 140904 | 19 |
| | ATGT | | T*+G*+T | | |
| 5062 | TTTGGTAGAATAAGA | A575060Hi | +T*+T*+T*G*G*T*A*G*A*A*T*A*A*G*A*C*+ | 140951 | 19 |
| | CTAC | | T*+A*+C | | |
| 5063 | CTTTGGTAGAATAAG | A575061Hi | +C*+T*+T*T*G*G*T*A*G*A*A*T*A*A*G*A*+ | 140952 | 19 |
| | ACTA | | C*+T*+A | | |
| 5064 | ACAACTTTGGTAGAA | A575062Hi | +A*+C*+A*A*C*T*T*T*G*G*T*A*G*A*A*T*+ | 140956 | 19 |
| | TAAG | | A*+A*+G | | |
| 5065 | CAACAACTTTGGTAG | A575063Hi | +C*+A*+A*C*A*A*C*T*T*T*G*G*T*A*G*+A* | 140959 | 18 |
| | AAT | | +A*+T | | |
| 5066 | TGCAACAACTTTGGT | A575064Hi | +T*+G*+C*A*A*C*A*A*C*T*T*T*G*G*T*A*+ | 140960 | 19 |
| | AGAA | | G*+A*+A | | |
| 5067 | AGAGAATGCAACAAC | A575065Hi | +A*+G*+A*G*A*A*T*G*C*A*A*C*A*A*C*T*+ | 140966 | 19 |
| | TTTG | | T*+T*+G | | |
| 5068 | CAACTAGAGAATGCA | A575066Hi | +C*+A*+A*C*T*A*G*A*G*A*A*T*G*C*A*A*+ | 140971 | 19 |
| | ACAA | | C*+A*+A | | |
| 5069 | CAACTAGAGAATGCA | A575067Hi | +C*+A*+A*C*T*A*G*A*G*A*A*T*G*C*A*+A* | 140972 | 18 |
| | ACA | | +C*+A | | |
| 5070 | AATTCAACTAGAGAA | A575068Hi | +A*+A*+T*T*C*A*A*C*T*A*G*A*G*A*A*T*+ | 140975 | 19 |
| | TGCA | | G*+C*+A | | |
| 5071 | CATACAATTCAACTA | A575069Hi | +C*+A*+T*A*C*A*A*T*T*C*A*A*C*T*A*G*+ | 140980 | 19 |
| | GAGA | | A*+G*+A | | |
| 5072 | TACATACAATTCAAC | A575070Hi | +T*+A*+C*A*T*A*C*A*A*T*T*C*A*A*C*T*+ | 140982 | 19 |
| | TAGA | | A*+G*+A | | |
| 5073 | GTACATACAATTCAA | A575071Hi | +G*+T*+A*C*A*T*A*C*A*A*T*T*C*A*A*C*+ | 140983 | 19 |
| | CTAG | | T*+A*+G | | |
| 5074 | CCAAAGTACATACAA | A575072Hi | +C*+C*+A*A*A*G*T*A*C*A*T*A*C*A*A*T*+ | 140988 | 19 |
| | TTCA | | T*+C*+A | | |
| 5075 | CTTCAGTTATAAGCA | A575073Hi | +C*+T*+T*C*A*G*T*T*A*T*A*A*G*C*A*A*+ | 141016 | 19 |
| | AGCT | | G*+C*+T | | |
| 5076 | TTCAGTTATAAGCAA | A575074Hi | +T*+T*+C*A*G*T*T*A*T*A*A*G*C*A*A*+G* | 141016 | 18 |
| | GCT | | +C*+T | | |
| 5077 | ACTTCAGTTATAAGC | A575075Hi | +A*+C*+T*T*C*A*G*T*T*A*T*A*A*G*C*A*+ | 141017 | 19 |
| | AAGC | | A*+G*+C | | |
| 5078 | CTTCAGTTATAAGCA | A575076Hi | +C*+T*+T*C*A*G*T*T*A*T*A*A*G*C*A*+A* | 141017 | 18 |
| | AGC | | +G*+C | | |
| 5079 | AACTTCAGTTATAAG | A575077Hi | +A*+A*+C*T*T*C*A*G*T*T*A*T*A*A*G*C*+ | 141018 | 19 |
| | CAAG | | A*+A*+G | | |
| 5080 | TAACTTCAGTTATAA | A575078Hi | +T*+A*+A*C*T*T*C*A*G*T*T*A*T*A*A*G*+ | 141019 | 19 |
| | GCAA | | C*+A*+A | | |
| 5081 | CCACTATAACTTCAG | A575079Hi | +C*+C*+A*C*T*A*T*A*A*C*T*T*C*A*G*T*+ | 141025 | 19 |
| | TTAT | | T*+A*+T | | |
| 5082 | TGGAGGCAGGAGAG | A575080Hi | +T*+G*+G*A*G*G*C*A*G*G*A*G*A*G*G*T* | 141126 | 19 |
| | GTACA | | +A*+C*+A | | |
| 5083 | AATGTGGTAGATGAT | A575081Hi | +A*+A*+T*G*T*G*G*T*A*G*A*T*G*A*T*G*+ | 141140 | 19 |
| | GGAG | | G*+A*+G | | |
| 5084 | GATCTGGTAGGAATG | A575082Hi | +G*+A*+T*C*T*G*G*T*A*G*G*A*A*T*G*T*+ | 141151 | 19 |
| | TGGT | | G*+G*+T | | |
| 5085 | GAAGAACTGGATGTT | A575083Hi | +G*+A*+A*G*A*A*C*T*G*G*A*T*G*T*T*C*+ | 141181 | 19 |
| | CATC | | A*+T*+C | | |
| 5086 | AGAAGAACTGGATGT | A575084Hi | +A*+G*+A*A*G*A*A*C*T*G*G*A*T*G*T*T*+ | 141182 | 19 |
| | TCAT | | C*+A*+T | | |
| 5087 | CTTAGATGGATGGAT | A575085Hi | +C*+T*+T*A*G*A*T*G*G*A*T*G*G*A*T*A*+ | 141604 | 19 |
| | ATAC | | T*+A*+C | | |
| 5088 | GGACACTTAGATGGA | A575086Hi | +G*+G*+A*C*A*C*T*T*A*G*A*T*G*G*A*T*+ | 141609 | 19 |
| | TGGA | | G*+G*+A | | |
| 5089 | AGTATTTAGATGGAC | A575087Hi | +A*+G*+T*A*T*T*T*A*G*A*T*G*G*A*C*A*+ | 141620 | 19 |
| | ACTT | | C*+T*+T | | |
| 5090 | ATGAGTATTTAGATG | A575088Hi | +A*+T*+G*A*G*T*A*T*T*T*A*G*A*T*G*G*+ | 141623 | 19 |
| | GACA | | A*+C*+A | | |
| 5091 | ATGAGTATTTAGATG | A575089Hi | +A*+T*+G*A*G*T*A*T*T*T*A*G*A*T*G*+G* | 141624 | 18 |
| | GAC | | +A*+C | | |
| 5092 | TATGAGTATTTAGAT | A575090Hi | +T*+A*+T*G*A*G*T*A*T*T*T*A*G*A*T*+G* | 141625 | 18 |
| | GGA | | +G*+A | | |
| 5093 | TAAGGTACAAATATG | A575091Hi | +T*+A*+A*G*G*T*A*C*A*A*A*T*A*T*G*A*+ | 141654 | 19 |
| | AGTA | | G*+T*+A | | |
| 5094 | AGTTCTTTGGATATG | A575092Hi | +A*+G*+T*T*C*T*T*T*G*G*A*T*A*T*G*G*+ | 141670 | 19 |
| | GTAA | | T*+A*+A | | |
| 5095 | AATTATGTGAGTCTG | A575093Hi | +A*+A*+T*T*A*T*G*T*G*A*G*T*C*T*G*A*+ | 141778 | 19 |
| | AGAA | | G*+A*+A | | |
| 5096 | AATTATGTGAGTCTG | A575094Hi | +A*+A*+T*T*A*T*G*T*G*A*G*T*C*T*G*+A* | 141779 | 18 |
| | AGA | | +G*+A | | |
| 5097 | GATCCACTCCTTGCT | A575095Hi | +G*+A*+T*C*C*A*C*T*C*C*T*T*G*C*T*G*+ | 141802 | 19 |
| | GCAA | | C*+A*+A | | |
| 5098 | ATCCACTCCTTGCTG | A575096Hi | +A*+T*+C*C*A*C*T*C*C*T*T*G*C*T*G*+C* | 141802 | 18 |
| | CAA | | +A*+A | | |
| 5099 | TGATCCACTCCTTGC | A575097Hi | +T*+G*+A*T*C*C*A*C*T*C*C*T*T*G*C*T*+ | 141803 | 19 |
| | TGCA | | G*+C*+A | | |
| 5100 | TGTGATCCACTCCTT | A575098Hi | +T*+G*+T*G*A*T*C*C*A*C*T*C*C*T*T*G*+ | 141805 | 19 |
| | GCTG | | C*+T*+G | | |
| 5101 | TCTGTGATCCACTCC | A575099Hi | +T*+C*+T*G*T*G*A*T*C*C*A*C*T*C*C*T*+ | 141807 | 19 |
| | TTGC | | T*+G*+C | | |
| 5102 | AATCTGTGATCCACT | A575100Hi | +A*+A*+T*C*T*G*T*G*A*T*C*C*A*C*T*+C* | 141810 | 18 |
| | CCT | | +C*+T | | |
| 5103 | ATACCTTGGAACTGA | A575101Hi | +A*+T*+A*C*C*T*T*G*G*A*A*C*T*G*A*A*+ | 141898 | 19 |
| | ATTC | | T*+T*+C | | |
| 5104 | CACAAATACCTTGGA | A575102Hi | +C*+A*+C*A*A*A*T*A*C*C*T*T*G*G*A*A*+ | 141903 | 19 |
| | ACTG | | C*+T*+G | | |
| 5105 | ACACAAATACCTTGG | A575103Hi | +A*+C*+A*C*A*A*A*T*A*C*C*T*T*G*G*A*+ | 141904 | 19 |
| | AACT | | A*+C*+T | | |
| 5106 | GACACAAATACCTTG | A575104Hi | +G*+A*+C*A*C*A*A*A*T*A*C*C*T*T*G*G*+ | 141905 | 19 |
| | GAAC | | A*+A*+C | | |
| 5107 | CCAATACCAGCATGA | A575105H | +C*+C*+A*A*T*A*C*C*A*G*C*A*T*G*A*C*+ | 142021 | 19 |
| | CATT | | A*+T*+T | | |
| 5108 | CCCAATACCAGCATG | A575106H | +C*+C*+C*A*A*T*A*C*C*A*G*C*A*T*G*A*+ | 142022 | 19 |
| | ACAT | | C*+A*+T | | |
| 5109 | CCCAATACCAGCATG | A575107H | +C*+C*+C*A*A*T*A*C*C*A*G*C*A*T*G*+A* | 142023 | 18 |
| | ACA | | +C*+A | | |
| 5110 | GCCCAATACCAGCAT | A575108H | +G*+C*+C*C*A*A*T*A*C*C*A*G*C*A*T*G*+ | 142023 | 19 |
| | GACA | | A*+C*+A | | |
| 5111 | TAGCCCAATACCAGC | A575109H | +T*+A*+G*C*C*C*A*A*T*A*C*C*A*G*C*A*+ | 142025 | 19 |
| | ATGA | | T*+G*+A | | |
| 5112 | CTGATTATACCATAT | A575110Hi | +C*+T*+G*A*T*T*A*T*A*C*C*A*T*A*T*G*+ | 142171 | 19 |
| | GTAA | | T*+A*+A | | |
| 5113 | CACTTCATATCACTT | A575111Hi | +C*+A*+C*T*T*C*A*T*A*T*C*A*C*T*T*G*+ | 142243 | 19 |
| | GTAA | | T*+A*+A | | |
| 5114 | TAACACTTCATATCA | A575112Hi | +T*+A*+A*C*A*C*T*T*C*A*T*A*T*C*A*C*+ | 142246 | 19 |
| | CTTG | | T*+T*+G | | |
| 5115 | GATTGACACCTCTAC | A575113Hi | +G*+A*+T*T*G*A*C*A*C*C*T*C*T*A*C*T*+ | 142283 | 19 |
| | TTCA | | T*+C*+A | | |
| 5116 | GCTAGTGATTGACAC | A575114Hi | +G*+C*+T*A*G*T*G*A*T*T*G*A*C*A*C*+C* | 142290 | 18 |
| | CTC | | +T*+C | | |
| 5117 | TGAGCTAGTGATTGA | A575115Hi | +T*+G*+A*G*C*T*A*G*T*G*A*T*T*G*A*C*+ | 142292 | 19 |
| | CACC | | A*+C*+C | | |
| 5118 | GAGCTAGTGATTGAC | A575116Hi | +G*+A*+G*C*T*A*G*T*G*A*T*T*G*A*C*+A* | 142292 | 18 |
| | ACC | | +C*+C | | |
| 5119 | TTGAGCTAGTGATTG | A575117Hi | +T*+T*+G*A*G*C*T*A*G*T*G*A*T*T*G*A*+ | 142293 | 19 |
| | ACAC | | C*+A*+C | | |
| 5120 | TAAGGTTGAGCTAGT | A575118Hi | +T*+A*+A*G*G*T*T*G*A*G*C*T*A*G*T*G*+ | 142298 | 19 |
| | GATT | | A*+T*+T | | |
| 5121 | TAAGGTTGAGCTAGT | A575119Hi | +T*+A*+A*G*G*T*T*G*A*G*C*T*A*G*T*+G* | 142299 | 18 |
| | GAT | | +A*+T | | |
| 5122 | TAAGGTTGAGCTAGT | A575120Hi | +T*+A*+A*G*G*T*T*G*A*G*C*T*A*G*+T*+G | 142300 | 17 |
| | GA | | *+A | | |
| 5123 | TTAAGGTTGAGCTAG | A575121Hi | +T*+T*+A*A*G*G*T*T*G*A*G*C*T*A*G*+T* | 142300 | 18 |
| | TGA | | +G*+A | | |
| 5124 | GTCAAGCAAGATTGT | A575122H | +G*+T*+C*A*A*G*C*A*A*G*A*T*T*G*T*A*+ | 142648 | 19 |
| | ATCT | | T*+C*+T | | |
| 5125 | GTCAAGCAAGATTGT | A575123H | +G*+T*+C*A*A*G*C*A*A*G*A*T*T*G*T*+A* | 142649 | 18 |
| | ATC | | +T*+C | | |
| 5126 | TGTGGAAGATTGATG | A575124H | +T*+G*+T*G*G*A*A*G*A*T*T*G*A*T*G*T*+ | 142672 | 19 |
| | TCCC | | C*+C*+C | | |
| 5127 | TGTGGAAGATTGATG | A575125H | +T*+G*+T*G*G*A*A*G*A*T*T*G*A*T*G*+T* | 142673 | 18 |
| | TCC | | +C*+C | | |
| 5128 | ATGTGGAAGATTGAT | A575126H | +A*+T*+G*T*G*G*A*A*G*A*T*T*G*A*T*G*+ | 142673 | 19 |
| | GTCC | | T*+C*+C | | |
| 5129 | TCATGTGGAAGATTG | A575127H | +T*+C*+A*T*G*T*G*G*A*A*G*A*T*T*G*A*+ | 142675 | 19 |
| | ATGT | | T*+G*+T | | |
| 5130 | TCATGTGGAAGATTG | A575128H | +T*+C*+A*T*G*T*G*G*A*A*G*A*T*T*G*+A* | 142676 | 18 |
| | ATG | | +T*+G | | |
| 5131 | TTCATGTGGAAGATT | A575129H | +T*+T*+C*A*T*G*T*G*G*A*A*G*A*T*T*G*+ | 142676 | 19 |
| | GATG | | A*+T*+G | | |
| 5132 | CTTCATGTGGAAGAT | A575130H | +C*+T*+T*C*A*T*G*T*G*G*A*A*G*A*T*T*+ | 142677 | 19 |
| | TGAT | | G*+A*+T | | |
| 5133 | GCACTTCATGTGGAA | A575131H | +G*+C*+A*C*T*T*C*A*T*G*T*G*G*A*A*G*+ | 142680 | 19 |
| | GATT | | A*+T*+T | | |
| 5134 | GCACTTCATGTGGAA | A575132H | +G*+C*+A*C*T*T*C*A*T*G*T*G*G*A*A*+G* | 142681 | 18 |
| | GAT | | +A*+T | | |
| 5135 | TGCACTTCATGTGGA | A575133H | +T*+G*+C*A*C*T*T*C*A*T*G*T*G*G*A*A*+ | 142681 | 19 |
| | AGAT | | G*+A*+T | | |
| 5136 | TGCACTTCATGTGGA | A575134H | +T*+G*+C*A*C*T*T*C*A*T*G*T*G*G*A*+A* | 142682 | 18 |
| | AGA | | +G*+A | | |
| 5137 | TTGCACTTCATGTGG | A575135H | +T*+T*+G*C*A*C*T*T*C*A*T*G*T*G*G*A*+ | 142682 | 19 |
| | AAGA | | A*+G*+A | | |
| 5138 | AAATTTTGCACTTCAT | A575136H | +A*+A*+A*T*T*T*T*G*C*A*C*T*T*C*A*T*+ | 142687 | 19 |
| | GTG | | G*+T*+G | | |
| 5139 | CTAAATTTTGCACTTC | A575137H | +C*+T*+A*A*A*T*T*T*T*G*C*A*C*T*T*C*+ | 142689 | 19 |
| | ATG | | A*+T*+G | | |
| 5140 | TTCAGCTAATTCTTTT | A575138H | +T*+T*+C*A*G*C*T*A*A*T*T*C*T*T*T*T*+ | 142724 | 19 |
| | CTC | | C*+T*+C | | |
| 5141 | TTCCTATCCAAAGAC | A575139H | +T*+T*+C*C*T*A*T*C*C*A*A*A*G*A*C*A*+ | 142782 | 19 |
| | AATT | | A*+T*+T | | |
| 5142 | CAAAAAATGTGAGTA | A575140H | +C*+A*+A*A*A*A*A*T*G*T*G*A*G*T*A*C*+ | 142851 | 19 |
| | CCCT | | C*+C*+T | | |
| 5143 | TGAGTACTTAAATTA | A575141H | +T*+G*+A*G*T*A*C*T*T*A*A*A*T*T*A*G*+ | 143036 | 19 |
| | GGTA | | G*+T*+A | | |
| 5144 | TGAGTACTTAAATTA | A575142H | +T*+G*+A*G*T*A*C*T*T*A*A*A*T*T*A*+G* | 143037 | 18 |
| | GGT | | +G*+T | | |
| 5145 | ATGAGTACTTAAATT | A575143H | +A*+T*+G*A*G*T*A*C*T*T*A*A*A*T*T*A*+ | 143037 | 19 |
| | AGGT | | G*+G*+T | | |
| 5146 | GTATGAGTACTTAAA | A575144H | +G*+T*+A*T*G*A*G*T*A*C*T*T*A*A*A*T*+ | 143039 | 19 |
| | TTAG | | T*+A*+G | | |
| 5147 | GTATGAGTACTTAAA | A575145H | +G*+T*+A*T*G*A*G*T*A*C*T*T*A*A*A*+T* | 143040 | 18 |
| | TTA | | +T*+A | | |
| 5148 | AGTATGAGTACTTAA | A575146H | +A*+G*+T*A*T*G*A*G*T*A*C*T*T*A*A*A*+ | 143040 | 19 |
| | ATTA | | T*+T*+A | | |
| 5149 | TTAGTATGAGTACTT | A575147H | +T*+T*+A*G*T*A*T*G*A*G*T*A*C*T*T*A*+ | 143042 | 19 |
| | AAAT | | A*+A*+T | | |
| 5150 | TTTAGTATGAGTACT | A575148H | +T*+T*+T*A*G*T*A*T*G*A*G*T*A*C*T*T*+ | 143043 | 19 |
| | TAAA | | A*+A*+A | | |
| 5151 | TTTAGTATGAGTACT | A575149H | +T*+T*+T*A*G*T*A*T*G*A*G*T*A*C*T*+T* | 143044 | 18 |
| | TAA | | +A*+A | | |
| 5152 | TTTTAGTATGAGTAC | A575150H | +T*+T*+T*T*A*G*T*A*T*G*A*G*T*A*C*T*+ | 143044 | 19 |
| | TTAA | | T*+A*+A | | |
| 5153 | TTTTAGTATGAGTAC | A575151H | +T*+T*+T*T*A*G*T*A*T*G*A*G*T*A*C*+T* | 143045 | 18 |
| | TTA | | +T*+A | | |
| 5154 | ATTTTAGTATGAGTA | A575152H | +A*+T*+T*T*T*A*G*T*A*T*G*A*G*T*A*C*+ | 143045 | 19 |
| | CTTA | | T*+T*+A | | |
| 5155 | AATTTTAGTATGAGT | A575153H | +A*+A*+T*T*T*T*A*G*T*A*T*G*A*G*T*A*+ | 143046 | 19 |
| | ACTT | | C*+T*+T | | |
| 5156 | TAAATTTTAGTATGA | A575154H | +T*+A*+A*A*T*T*T*T*A*G*T*A*T*G*A*G*+ | 143048 | 19 |
| | GTAC | | T*+A*+C | | |
| 5157 | CGGCCTTATAAATTT | A575155H | +C*+G*+G*C*C*T*T*A*T*A*A*A*T*T*T*T*+ | 143056 | 19 |
| | TAGT | | A*+G*+T | | |
| 5158 | TATCGGCCTTATAAA | A575156H | +T*+A*+T*C*G*G*C*C*T*T*A*T*A*A*A*T*+ | 143059 | 19 |
| | TTTT | | T*+T*+T | | |
| 5159 | TATCGGCCTTATAAA | A575157H | +T*+A*+T*C*G*G*C*C*T*T*A*T*A*A*A*+T* | 143060 | 18 |
| | TTT | | +T*+T | | |
| 5160 | TTATCGGCCTTATAA | A575158H | +T*+T*+A*T*C*G*G*C*C*T*T*A*T*A*A*A*+ | 143060 | 19 |
| | ATTT | | T*+T*+T | | |
| 5161 | ATTATCGGCCTTATA | A575159H | +A*+T*+T*A*T*C*G*G*C*C*T*T*A*T*A*A*+ | 143061 | 19 |
| | AATT | | A*+T*+T | | |
| 5162 | ATTATCGGCCTTATA | A575160H | +A*+T*+T*A*T*C*G*G*C*C*T*T*A*T*A*+A* | 143062 | 18 |
| | AAT | | +A*+T | | |
| 5163 | AATTATCGGCCTTAT | A575161H | +A*+A*+T*T*A*T*C*G*G*C*C*T*T*A*T*A*+ | 143062 | 19 |
| | AAAT | | A*+A*+T | | |
| 5164 | AATTATCGGCCTTAT | A575162H | +A*+A*+T*T*A*T*C*G*G*C*C*T*T*A*T*+A* | 143063 | 18 |
| | AAA | | +A*+A | | |
| 5165 | AAATTATCGGCCTTA | A575163H | +A*+A*+A*T*T*A*T*C*G*G*C*C*T*T*A*T*+ | 143063 | 19 |
| | TAAA | | A*+A*+A | | |
| 5166 | AATTATCGGCCTTAT | A575164H | +A*+A*+T*T*A*T*C*G*G*C*C*T*T*A*+T*+A | 143064 | 17 |
| | AA | | *+A | | |
| 5167 | AAATTATCGGCCTTA | A575165H | +A*+A*+A*T*T*A*T*C*G*G*C*C*T*T*A*+T* | 143064 | 18 |
| | TAA | | +A*+A | | |
| 5168 | AAAATTATCGGCCTT | A575166H | +A*+A*+A*A*T*T*A*T*C*G*G*C*C*T*T*A*+ | 143064 | 19 |
| | ATAA | | T*+A*+A | | |
| 5169 | CAAAAAATTATCGGC | A575167H | +C*+A*+A*A*A*A*A*T*T*A*T*C*G*G*C*C*+ | 143067 | 19 |
| | CTTA | | T*+T*+A | | |
| 5170 | GGAAACTGTCTTAAT | A575168H | +G*+G*+A*A*A*C*T*G*T*C*T*T*A*A*T*A*+ | 143185 | 19 |
| | ATTT | | T*+T*+T | | |
| 5171 | GGGAAACTGTCTTAA | A575169H | +G*+G*+G*A*A*A*C*T*G*T*C*T*T*A*A*T*+ | 143186 | 19 |
| | TATT | | A*+T*+T | | |
| 5172 | CATATATGATAGTGC | A575170H | +C*+A*+T*A*T*A*T*G*A*T*A*G*T*G*C*A*+ | 143235 | 19 |
| | ATTT | | T*+T*+T | | |
| 5173 | AAGCATATATGATAG | A575171H | +A*+A*+G*C*A*T*A*T*A*T*G*A*T*A*G*T*+ | 143238 | 19 |
| | TGCA | | G*+C*+A | | |
| 5174 | CAAGCATATATGATA | A575172H | +C*+A*+A*G*C*A*T*A*T*A*T*G*A*T*A*G*+ | 143239 | 19 |
| | GTGC | | T*+G*+C | | |
| 5175 | TTCACATACCAACAA | A575173H | +T*+T*+C*A*C*A*T*A*C*C*A*A*C*A*A*A*+ | 143289 | 19 |
| | AGGA | | G*+G*+A | | |
| 5176 | TTCACATACCAACAA | A575174H | +T*+T*+C*A*C*A*T*A*C*C*A*A*C*A*A*+A* | 143290 | 18 |
| | AGG | | +G*+G | | |
| 5177 | ATTCACATACCAACA | A575175H | +A*+T*+T*C*A*C*A*T*A*C*C*A*A*C*A*A*+ | 143290 | 19 |
| | AAGG | | A*+G*+G | | |
| 5178 | GTTGTGTAAGATGCA | A575176H | +G*+T*+T*G*T*G*T*A*A*G*A*T*G*C*A*C*+ | 143329 | 19 |
| | CTGT | | T*+G*+T | | |
| 5179 | GAAGTTGTGTAAGAT | A575177H | +G*+A*+A*G*T*T*G*T*G*T*A*A*G*A*T*G*+ | 143332 | 19 |
| | GCAC | | C*+A*+C | | |
| 5180 | AGTGAAGTTGTGTAA | A575178H | +A*+G*+T*G*A*A*G*T*T*G*T*G*T*A*A*G*+ | 143335 | 19 |
| | GATG | | A*+T*+G | | |
| 5181 | AGTACTTTTAATGGA | A575179H | +A*+G*+T*A*C*T*T*T*T*A*A*T*G*G*A*G*+ | 143366 | 19 |
| | GTTT | | T*+T*+T | | |
| 5182 | GACAGTATGTCATGT | A575180H | +G*+A*+C*A*G*T*A*T*G*T*C*A*T*G*T*T*+ | 143390 | 19 |
| | TTTT | | T*+T*+T | | |
| 5183 | TTTGACAGTATGTCA | A575181H | +T*+T*+T*G*A*C*A*G*T*A*T*G*T*C*A*T*+ | 143393 | 19 |
| | TGTT | | G*+T*+T | | |
| 5184 | ACTTTGACAGTATGT | A575182H | +A*+C*+T*T*T*G*A*C*A*G*T*A*T*G*T*C*+ | 143395 | 19 |
| | CATG | | A*+T*+G | | |
| 5185 | GACTTTGACAGTATG | A575183H | +G*+A*+C*T*T*T*G*A*C*A*G*T*A*T*G*T*+ | 143396 | 19 |
| | TCAT | | C*+A*+T | | |
| 5186 | GGACTTTGACAGTAT | A575184H | +G*+G*+A*C*T*T*T*G*A*C*A*G*T*A*T*G*+ | 143397 | 19 |
| | GTCA | | T*+C*+A | | |
| 5187 | GGACTTTGACAGTAT | A575185H | +G*+G*+A*C*T*T*T*G*A*C*A*G*T*A*T*+G* | 143398 | 18 |
| | GTC | | +T*+C | | |
| 5188 | GAGGACTTTGACAGT | A575186H | +G*+A*+G*G*A*C*T*T*T*G*A*C*A*G*T*A*+ | 143399 | 19 |
| | ATGT | | T*+G*+T | | |
| 5189 | ATGAGGACTTTGACA | A575187H | +A*+T*+G*A*G*G*A*C*T*T*T*G*A*C*A*G*+ | 143401 | 19 |
| | GTAT | | T*+A*+T | | |
| 5190 | ATATGAGGACTTTGA | A575188H | +A*+T*+A*T*G*A*G*G*A*C*T*T*T*G*A*C*+ | 143403 | 19 |
| | CAGT | | A*+G*+T | | |
| 5191 | GATATGAGGACTTTG | A575189H | +G*+A*+T*A*T*G*A*G*G*A*C*T*T*T*G*A*+ | 143404 | 19 |
| | ACAG | | C*+A*+G | | |
| 5192 | CCTAGATATGAGGAC | A575190H | +C*+C*+T*A*G*A*T*A*T*G*A*G*G*A*C*T*+ | 143408 | 19 |
| | TTTG | | T*+T*+G | | |
| 5193 | TCCTAGATATGAGGA | A575191H | +T*+C*+C*T*A*G*A*T*A*T*G*A*G*G*A*C*+ | 143409 | 19 |
| | CTTT | | T*+T*+T | | |
| 5194 | TCCTAGATATGAGGA | A575192H | +T*+C*+C*T*A*G*A*T*A*T*G*A*G*G*A*+C* | 143410 | 18 |
| | CTT | | +T*+T | | |
| 5195 | GTATTTACACTAATTA | A575193H | +G*+T*+A*T*T*T*A*C*A*C*T*A*A*T*T*A*+ | 143531 | 19 |
| | TAC | | T*+A*+C | | |
| 5196 | ACACTGTATTTACAC | A575194H | +A*+C*+A*C*T*G*T*A*T*T*T*A*C*A*C*T*+ | 143536 | 19 |
| | TAAT | | A*+A*+T | | |
| 5197 | GAAGGACTGAACACT | A575195H | +G*+A*+A*G*G*A*C*T*G*A*A*C*A*C*T*G*+ | 143546 | 19 |
| | GTAT | | T*+A*+T | | |
| 5198 | GAAGGACTGAACACT | A575196H | +G*+A*+A*G*G*A*C*T*G*A*A*C*A*C*T*+G* | 143547 | 18 |
| | GTA | | +T*+A | | |
| 5199 | TGAAGGACTGAACAC | A575197H | +T*+G*+A*A*G*G*A*C*T*G*A*A*C*A*C*T*+ | 143547 | 19 |
| | TGTA | | G*+T*+A | | |
| 5200 | TTGAAGGACTGAACA | A575198H | +T*+T*+G*A*A*G*G*A*C*T*G*A*A*C*A*C*+ | 143548 | 19 |
| | CTGT | | T*+G*+T | | |
| 5201 | TTGAAGGACTGAACA | A575199H | +T*+T*+G*A*A*G*G*A*C*T*G*A*A*C*A*+C* | 143549 | 18 |
| | CTG | | +T*+G | | |
| 5202 | CTTGAAGGACTGAAC | A575200H | +C*+T*+T*G*A*A*G*G*A*C*T*G*A*A*C*A*+ | 143549 | 19 |
| | ACTG | | C*+T*+G | | |
| 5203 | TCACTTGAAGGACTG | A575201H | +T*+C*+A*C*T*T*G*A*A*G*G*A*C*T*G*A*+ | 143552 | 19 |
| | AACA | | A*+C*+A | | |
| 5204 | TCACTTGAAGGACTG | A575202H | +T*+C*+A*C*T*T*G*A*A*G*G*A*C*T*G*+A* | 143553 | 18 |
| | AAC | | +A*+C | | |
| 5205 | ATCACTTGAAGGACT | A575203H | +A*+T*+C*A*C*T*T*G*A*A*G*G*A*C*T*G*+ | 143553 | 19 |
| | GAAC | | A*+A*+C | | |
| 5206 | ATATCACTTGAAGGA | A575204H | +A*+T*+A*T*C*A*C*T*T*G*A*A*G*G*A*C*+ | 143555 | 19 |
| | CTGA | | T*+G*+A | | |
| 5207 | AATATCACTTGAAGG | A575205H | +A*+A*+T*A*T*C*A*C*T*T*G*A*A*G*G*A*+ | 143556 | 19 |
| | ACTG | | C*+T*+G | | |
| 5208 | AAATATCACTTGAAG | A575206H | +A*+A*+A*T*A*T*C*A*C*T*T*G*A*A*G*G*+ | 143557 | 19 |
| | GACT | | A*+C*+T | | |
| 5209 | AGATGAATATAAGCA | A575207H | +A*+G*+A*T*G*A*A*T*A*T*A*A*G*C*A*T*+ | 143626 | 19 |
| | TTAG | | T*+A*+G | | |
| 5210 | AAGATGAATATAAGC | A575208H | +A*+A*+G*A*T*G*A*A*T*A*T*A*A*G*C*A*+ | 143627 | 19 |
| | ATTA | | T*+T*+A | | |
| 5211 | GGAAAAGATGAATAT | A575209H | +G*+G*+A*A*A*A*G*A*T*G*A*A*T*A*T*A*+ | 143631 | 19 |
| | AAGC | | A*+G*+C | | |
| 5212 | CATCACAAATTTAGG | A575210H | +C*+A*+T*C*A*C*A*A*A*T*T*T*A*G*G*G*+ | 143645 | 19 |
| | GAAA | | A*+A*+A | | |
| 5213 | GCATCACAAATTTAG | A575211H | +G*+C*+A*T*C*A*C*A*A*A*T*T*T*A*G*G*+ | 143646 | 19 |
| | GGAA | | G*+A*+A | | |
| 5214 | CAGCATCACAAATTT | A575212H | +C*+A*+G*C*A*T*C*A*C*A*A*A*T*T*T*A*+ | 143648 | 19 |
| | AGGG | | G*+G*+G | | |
| 5215 | GCAGCATCACAAATT | A575213H | +G*+C*+A*G*C*A*T*C*A*C*A*A*A*T*T*T*+ | 143649 | 19 |
| | TAGG | | A*+G*+G | | |
| 5216 | TGCAGCATCACAAAT | A575214H | +T*+G*+C*A*G*C*A*T*C*A*C*A*A*A*T*T*+ | 143650 | 19 |
| | TTAG | | T*+A*+G | | |
| 5217 | CTGCAGCATCACAAA | A575215H | +C*+T*+G*C*A*G*C*A*T*C*A*C*A*A*A*T*+ | 143651 | 19 |
| | TTTA | | T*+T*+A | | |
| 5218 | GATCTGCAGCATCAC | A575216H | +G*+A*+T*C*T*G*C*A*G*C*A*T*C*A*C*A*+ | 143654 | 19 |
| | AAAT | | A*+A*+T | | |
| 5219 | AGGATCTGCAGCATC | A575217H | +A*+G*+G*A*T*C*T*G*C*A*G*C*A*T*C*A*+ | 143656 | 19 |
| | ACAA | | C*+A*+A | | |
| 5220 | AGGATCTGCAGCATC | A575218H | +A*+G*+G*A*T*C*T*G*C*A*G*C*A*T*C*+A* | 143657 | 18 |
| | ACA | | +C*+A | | |
| 5221 | TAGGATCTGCAGCAT | A575219H | +T*+A*+G*G*A*T*C*T*G*C*A*G*C*A*T*C*+ | 143657 | 19 |
| | CACA | | A*+C*+A | | |
| 5222 | TAGGATCTGCAGCAT | A575220H | +T*+A*+G*G*A*T*C*T*G*C*A*G*C*A*T*+C* | 143658 | 18 |
| | CAC | | +A*+C | | |
| 5223 | GTAGGATCTGCAGCA | A575221H | +G*+T*+A*G*G*A*T*C*T*G*C*A*G*C*A*T*+ | 143658 | 19 |
| | TCAC | | C*+A*+C | | |
| 5224 | ATGTAGGATCTGCAG | A575222H | +A*+T*+G*T*A*G*G*A*T*C*T*G*C*A*G*C*+ | 143660 | 19 |
| | CATC | | A*+T*+C | | |
| 5225 | ATGATGTAGGATCTG | A575223H | +A*+T*+G*A*T*G*T*A*G*G*A*T*C*T*G*C*+ | 143663 | 19 |
| | CAGC | | A*+G*+C | | |
| 5226 | GCTGTGGAATTCTAT | A575224H | +G*+C*+T*G*T*G*G*A*A*T*T*C*T*A*T*A*+ | 143709 | 19 |
| | AATT | | A*+T*+T | | |
| 5227 | GGAGCTGTGGAATTC | A575225H | +G*+G*+A*G*C*T*G*T*G*G*A*A*T*T*C*T*+ | 143712 | 19 |
| | TATA | | A*+T*+A | | |
| 5228 | GGTAGGAGCTGTGGA | A575226H | +G*+G*+T*A*G*G*A*G*C*T*G*T*G*G*A*A* | 143716 | 19 |
| | ATTC | | +T*+T*+C | | |
| 5229 | TGGTAGGAGCTGTGG | A575227H | +T*+G*+G*T*A*G*G*A*G*C*T*G*T*G*G*A* | 143717 | 19 |
| | AATT | | +A*+T*+T | | |
| 5230 | CTTGGTAGGAGCTGT | A575228H | +C*+T*+T*G*G*T*A*G*G*A*G*C*T*G*T*G*+ | 143719 | 19 |
| | GGAA | | G*+A*+A | | |
| 5231 | TCTTGGTAGGAGCTG | A575229H | +T*+C*+T*T*G*G*T*A*G*G*A*G*C*T*G*T*+ | 143720 | 19 |
| | TGGA | | G*+G*+A | | |
| 5232 | GTCTTGGTAGGAGCT | A575230H | +G*+T*+C*T*T*G*G*T*A*G*G*A*G*C*T*G*+ | 143721 | 19 |
| | GTGG | | T*+G*+G | | |
| 5233 | TGGTCTTGGTAGGAG | A575231H | +T*+G*+G*T*C*T*T*G*G*T*A*G*G*A*G*C*+ | 143723 | 19 |
| | CTGT | | T*+G*+T | | |
| 5234 | ATGGTCTTGGTAGGA | A575232H | +A*+T*+G*G*T*C*T*T*G*G*T*A*G*G*A*G*+ | 143724 | 19 |
| | GCTG | | C*+T*+G | | |
| 5235 | CATGGTCTTGGTAGG | A575233H | +C*+A*+T*G*G*T*C*T*T*G*G*T*A*G*G*A*+ | 143725 | 19 |
| | AGCT | | G*+C*+T | | |
| 5236 | TCATGGTCTTGGTAG | A575234H | +T*+C*+A*T*G*G*T*C*T*T*G*G*T*A*G*G*+ | 143726 | 19 |
| | GAGC | | A*+G*+C | | |
| 5237 | CTCATGGTCTTGGTA | A575235H | +C*+T*+C*A*T*G*G*T*C*T*T*G*G*T*A*G*+ | 143727 | 19 |
| | GGAG | | G*+A*+G | | |
| 5238 | CTCATGGTCTTGGTA | A575236H | +C*+T*+C*A*T*G*G*T*C*T*T*G*G*T*A*+G* | 143728 | 18 |
| | GGA | | +G*+A | | |
| 5239 | CCTCATGGTCTTGGT | A575237H | +C*+C*+T*C*A*T*G*G*T*C*T*T*G*G*T*A*+ | 143728 | 19 |
| | AGGA | | G*+G*+A | | |
| 5240 | TCCTCATGGTCTTGG | A575238H | +T*+C*+C*T*C*A*T*G*G*T*C*T*T*G*G*T*+ | 143729 | 19 |
| | TAGG | | A*+G*+G | | |
| 5241 | CAGCAACTGAAAAGT | A575239H | +C*+A*+G*C*A*A*C*T*G*A*A*A*A*G*T*G*+ | 143756 | 19 |
| | GTTA | | T*+T*+A | | |
| 5242 | TCTCCTTCAGCAACT | A575240H | +T*+C*+T*C*C*T*T*C*A*G*C*A*A*C*T*G*+ | 143763 | 19 |
| | GAAA | | A*+A*+A | | |
| 5243 | CCATCATAACTAAAG | A575241H | +C*+C*+A*T*C*A*T*A*A*C*T*A*A*A*G*+C* | 143785 | 18 |
| | CTC | | +T*+C | | |
| 5244 | TCCATCATAACTAAA | A575242H | +T*+C*+C*A*T*C*A*T*A*A*C*T*A*A*A*G*+ | 143785 | 19 |
| | GCTC | | C*+T*+C | | |
| 5245 | ATCCATCATAACTAA | A575243H | +A*+T*+C*C*A*T*C*A*T*A*A*C*T*A*A*A*+ | 143786 | 19 |
| | AGCT | | G*+C*+T | | |
| 5246 | TATCCATCATAACTA | A575244H | +T*+A*+T*C*C*A*T*C*A*T*A*A*C*T*A*A*+ | 143787 | 19 |
| | AAGC | | A*+G*+C | | |
| 5247 | TTATCCATCATAACTA | A575245H | +T*+T*+A*T*C*C*A*T*C*A*T*A*A*C*T*A*+ | 143788 | 19 |
| | AAG | | A*+A*+G | | |
| 5248 | TATTTTTATCCATCAT | A575246H | +T*+A*+T*T*T*T*T*A*T*C*C*A*T*C*A*T*+ | 143793 | 19 |
| | AAC | | A*+A*+C | | |
| 5249 | CCTAGGGTGGCAGAT | A575247H | +C*+C*+T*A*G*G*G*T*G*G*C*A*G*A*T*A*+ | 143807 | 19 |
| | ATTT | | T*+T*+T | | |
| 5250 | TGGAAGCCTAGGGTG | A575248H | +T*+G*+G*A*A*G*C*C*T*A*G*G*G*T*G*G* | 143813 | 19 |
| | GCAG | | +C*+A*+G | | |
| 5251 | GCTATGTAAACAATT | A575249H | +G*+C*+T*A*T*G*T*A*A*A*C*A*A*T*T*T*+ | 143839 | 19 |
| | TAAG | | A*+A*+G | | |
| 5252 | AGCTATGTAAACAAT | A575250H | +A*+G*+C*T*A*T*G*T*A*A*A*C*A*A*T*T*+ | 143840 | 19 |
| | TTAA | | T*+A*+A | | |
| 5253 | AAGCTATGTAAACAA | A575251H | +A*+A*+G*C*T*A*T*G*T*A*A*A*C*A*A*T*+ | 143841 | 19 |
| | TTTA | | T*+T*+A | | |
| 5254 | GTAAGCTATGTAAAC | A575252H | +G*+T*+A*A*G*C*T*A*T*G*T*A*A*A*C*A*+ | 143843 | 19 |
| | AATT | | A*+T*+T | | |
| 5255 | GGTAAGCTATGTAAA | A575253H | +G*+G*+T*A*A*G*C*T*A*T*G*T*A*A*A*C*+ | 143844 | 19 |
| | CAAT | | A*+A*+T | | |
| 5256 | TGGTAAGCTATGTAA | A575254H | +T*+G*+G*T*A*A*G*C*T*A*T*G*T*A*A*A*+ | 143845 | 19 |
| | ACAA | | C*+A*+A | | |
| 5257 | TGTGGTAAGCTATGT | A575255H | +T*+G*+T*G*G*T*A*A*G*C*T*A*T*G*T*A*+ | 143847 | 19 |
| | AAAC | | A*+A*+C | | |
| 5258 | TATTTGGCCCTGATA | A575256H | +T*+A*+T*T*T*G*G*C*C*C*T*G*A*T*A*C*+ | 143869 | 19 |
| | CTCC | | T*+C*+C | | |
| 5259 | TAGGTATTTGGCCCT | A575257H | +T*+A*+G*G*T*A*T*T*T*G*G*C*C*C*T*G*+ | 143873 | 19 |
| | GATA | | A*+T*+A | | |
| 5260 | ACATAGGTATTTGGC | A575258H | +A*+C*+A*T*A*G*G*T*A*T*T*T*G*G*C*C*+ | 143876 | 19 |
| | CCTG | | C*+T*+G | | |
| 5261 | ACATAGGTATTTGGC | A575259H | +A*+C*+A*T*A*G*G*T*A*T*T*T*G*G*C*+C* | 143877 | 18 |
| | CCT | | +C*+T | | |
| 5262 | TACATAGGTATTTGG | A575260H | +T*+A*+C*A*T*A*G*G*T*A*T*T*T*G*G*C*+ | 143877 | 19 |
| | CCCT | | C*+C*+T | | |
| 5263 | TACATAGGTATTTGG | A575261H | +T*+A*+C*A*T*A*G*G*T*A*T*T*T*G*G*+C* | 143878 | 18 |
| | CCC | | +C*+C | | |
| 5264 | TTACATAGGTATTTG | A575262H | +T*+T*+A*C*A*T*A*G*G*T*A*T*T*T*G*G*+ | 143878 | 19 |
| | GCCC | | C*+C*+C | | |
| 5265 | TTACATAGGTATTTG | A575263H | +T*+T*+A*C*A*T*A*G*G*T*A*T*T*T*G*+G* | 143879 | 18 |
| | GCC | | +C*+C | | |
| 5266 | ATTACATAGGTATTT | A575264H | +A*+T*+T*A*C*A*T*A*G*G*T*A*T*T*T*G*+ | 143879 | 19 |
| | GGCC | | G*+C*+C | | |
| 5267 | ATTACATAGGTATTT | A575265H | +A*+T*+T*A*C*A*T*A*G*G*T*A*T*T*T*+G* | 143880 | 18 |
| | GGC | | +G*+C | | |
| 5268 | TATTACATAGGTATTT | A575266H | +T*+A*+T*T*A*C*A*T*A*G*G*T*A*T*T*T*+ | 143880 | 19 |
| | GGC | | G*+G*+C | | |
| 5269 | CTCAAATTATTACATA | A575267H | +C*+T*+C*A*A*A*T*T*A*T*T*A*C*A*T*A*+ | 143887 | 19 |
| | GGT | | G*+G*+T | | |
| 5270 | CCTCAAATTATTACAT | A575268H | +C*+C*+T*C*A*A*A*T*T*A*T*T*A*C*A*T*+ | 143888 | 19 |
| | AGG | | A*+G*+G | | |
| 5271 | CAGGGCCAAAGAATT | A575269H | +C*+A*+G*G*G*C*C*A*A*A*G*A*A*T*T*T*+ | 143935 | 19 |
| | TACC | | A*+C*+C | | |
| 5272 | TCAGGGCCAAAGAAT | A575270H | +T*+C*+A*G*G*G*C*C*A*A*A*G*A*A*T*T*+ | 143936 | 19 |
| | TTAC | | T*+A*+C | | |
| 5273 | GGTCAGGGCCAAAGA | A575271H | +G*+G*+T*C*A*G*G*G*C*C*A*A*A*G*A*A* | 143938 | 19 |
| | ATTT | | +T*+T*+T | | |
| 5274 | TGGTCAGGGCCAAAG | A575272H | +T*+G*+G*T*C*A*G*G*G*C*C*A*A*A*G*A* | 143939 | 19 |
| | AATT | | +A*+T*+T | | |
| 5275 | CTGGTCAGGGCCAAA | A575273H | +C*+T*+G*G*T*C*A*G*G*G*C*C*A*A*A*G* | 143940 | 19 |
| | GAAT | | +A*+A*+T | | |
| 5276 | TACTGGTCAGGGCCA | A575274H | +T*+A*+C*T*G*G*T*C*A*G*G*G*C*C*A*A*+ | 143942 | 19 |
| | AAGA | | A*+G*+A | | |
| 5277 | ATGAATACTGGTCAG | A575275H | +A*+T*+G*A*A*T*A*C*T*G*G*T*C*A*G*+G* | 143948 | 18 |
| | GGC | | +G*+C | | |
| 5278 | AATGAATACTGGTCA | A575276H | +A*+A*+T*G*A*A*T*A*C*T*G*G*T*C*A*G*+ | 143948 | 19 |
| | GGGC | | G*+G*+C | | |
| 5279 | GTCCTATCACAGGGA | A575277H | +G*+T*+C*C*T*A*T*C*A*C*A*G*G*G*A*+A* | 143976 | 18 |
| | ATT | | +T*+T | | |
| 5280 | TGTCCTATCACAGGG | A575278H | +T*+G*+T*C*C*T*A*T*C*A*C*A*G*G*G*A*+ | 143976 | 19 |
| | AATT | | A*+T*+T | | |
| 5281 | TTGTCCTATCACAGG | A575279H | +T*+T*+G*T*C*C*T*A*T*C*A*C*A*G*G*G*+ | 143977 | 19 |
| | GAAT | | A*+A*+T | | |
| 5282 | GTTGTCCTATCACAG | A575280H | +G*+T*+T*G*T*C*C*T*A*T*C*A*C*A*G*G*+ | 143978 | 19 |
| | GGAA | | G*+A*+A | | |
| 5283 | GTTGTCCTATCACAG | A575281H | +G*+T*+T*G*T*C*C*T*A*T*C*A*C*A*G*+G* | 143979 | 18 |
| | GGA | | +G*+A | | |
| 5284 | AGTTGTCCTATCACA | A575282H | +A*+G*+T*T*G*T*C*C*T*A*T*C*A*C*A*G*+ | 143979 | 19 |
| | GGGA | | G*+G*+A | | |
| 5285 | AGTTGTCCTATCACA | A575283H | +A*+G*+T*T*G*T*C*C*T*A*T*C*A*C*A*+G* | 143980 | 18 |
| | GGG | | +G*+G | | |
| 5286 | TAGTTGTCCTATCAC | A575284H | +T*+A*+G*T*T*G*T*C*C*T*A*T*C*A*C*A*+ | 143980 | 19 |
| | AGGG | | G*+G*+G | | |
| 5287 | ACTAGTTGTCCTATC | A575285H | +A*+C*+T*A*G*T*T*G*T*C*C*T*A*T*C*A*+ | 143982 | 19 |
| | ACAG | | C*+A*+G | | |
| 5288 | ACTAGTTGTCCTATC | A575286H | +A*+C*+T*A*G*T*T*G*T*C*C*T*A*T*C*+A* | 143983 | 18 |
| | ACA | | +C*+A | | |
| 5289 | AAATGCCATAAGTTC | A575287H | +A*+A*+A*T*G*C*C*A*T*A*A*G*T*T*C*T*+ | 144013 | 19 |
| | TGAG | | G*+A*+G | | |
| 5290 | CACTGGAACAAAGAG | A575288H | +C*+A*+C*T*G*G*A*A*C*A*A*A*G*A*G*C*+ | 144161 | 19 |
| | CTCT | | T*+C*+T | | |
| 5291 | AACACTGGAACAAAG | A575289H | +A*+A*+C*A*C*T*G*G*A*A*C*A*A*A*G*A*+ | 144163 | 19 |
| | AGCT | | G*+C*+T | | |
| 5292 | TAACACTGGAACAAA | A575290H | +T*+A*+A*C*A*C*T*G*G*A*A*C*A*A*A*G*+ | 144164 | 19 |
| | GAGC | | A*+G*+C | | |
| 5293 | ATAACACTGGAACAA | A575291H | +A*+T*+A*A*C*A*C*T*G*G*A*A*C*A*A*A*+ | 144165 | 19 |
| | AGAG | | G*+A*+G | | |
| 5294 | AAAGAGATAACACTG | A575292H | +A*+A*+A*G*A*G*A*T*A*A*C*A*C*T*G*G*+ | 144171 | 19 |
| | GAAC | | A*+A*+C | | |
| 5295 | TGAAAGAGATAACAC | A575293H | +T*+G*+A*A*A*G*A*G*A*T*A*A*C*A*C*T*+ | 144173 | 19 |
| | TGGA | | G*+G*+A | | |
| 5296 | CAGCATCATGCAAGC | A575294Hi | +C*+A*+G*C*A*T*C*A*T*G*C*A*A*G*C*A*+ | 78525 | 19 |
| | AGCA | | G*+C*+A | 84782 | |
| 5297 | GTTTGAAGGAATAGC | A575295Hi | +G*+T*+T*T*G*A*A*G*G*A*A*T*A*G*C*+T* | 78478 | 18 |
| | TGA | | +G*+A | 84735 | |
| 5298 | CAGCATCATGCAAGC | A575296Hi | +C*+A*+G*C*A*T*C*A*T*G*C*A*A*G*C*+A* | 78526 | 18 |
| | AGC | | +G*+C | 84783 | |
| 5299 | GCAGCATCATGCAAG | A575297Hi | +G*+C*+A*G*C*A*T*C*A*T*G*C*A*A*G*+C* | 78527 | 18 |
| | CAG | | +A*+G | 84784 | |
| 5300 | TGCAGCATCATGCAA | A575298Hi | +T*+G*+C*A*G*C*A*T*C*A*T*G*C*A*A*G*+ | 78527 | 19 |
| | GCAG | | C*+A*+G | 84784 | |
| 5301 | TTGCAGCATCATGCA | A575299Hi | +T*+T*+G*C*A*G*C*A*T*C*A*T*G*C*A*A*+ | 78528 | 19 |
| | AGCA | | G*+C*+A | 84785 | |
| 5302 | | Neg1 | +C*+G*+T*T*T*A*G*G*C*T*A*T*G*T*A*+C* | | 18 |
| | | | +T*+T | | |
| 5303 | | R01002 | +T*+A*+C*G*C*G*C*G*G*T*T*G*T*+T*+T*+ | | 16 |
| | | | A | | |
| 5304 | | R01011 | +G*+A*+T*C*A*T*T*C*G*C*G*G*A*C*+A*+A | | 17 |
| | | | *+C | | |
| 5305 | | R01025 | +A*+T*+A*T*G*T*G*T*C*G*C*G*A*T*T*G*+ | | 19 |
| | | | G*+A*+G | | |

The ASOs of the present invention hybridize, for example, with hybridizing active regions. Such regions comprise or consist, for example, of positions 1 to 500, 501 to 1000, 1001 to 1500, 1501 to 2000, 2001 to 2500, 3501 to 4000, 4001 to 4500, 4501 to 5000, 5001 to 5500, 5001 to 6000, 6001 to 6500, 6501 to 7000, 7001 to 7500, 7501 to 8000, 8001 to 8500, 8501 to 9000, 9001 to 10000, 10501 to 11000, 11001 to 11500, 11501 to 12000, 12001 to 12500, 12501 to 13000, 13001 to 13500, 13501 to 1400, 14501 to 15000, 15001 to 15500, 15501 to 16000, 16001 to 16500, 16501 to 17000, 17001 to 17500, 17501 to 18000, 18001 to 18500, 18501 to 19000, 19001 to 19500, 20001 to 20500, 20501 to 21000, 21001 to 21500, 21501 to 22000, 22001 to 22500, 23001 to 23500, 23501 to 24000, 24001 to 24500, 24501 to 25000, 25001 to 25500, 26001 to 26500, 26501 to 27000, 27001 to 27500, 27501 to 28000, 28001 to 28500, 29001 to 29500, 29501 to 30000, 30001 to 30500, 30501 to 31000, 31001 to 31500, 32001 to 32500, 33001 to 33500, 33501 to 34000, 34001 to 34500, 34501 to 35000, 35001 to 35500, 35501 to 36000, 36001 to 36500, 36501 to 37000, 37001 to 37500, 37501 to 38000, 38001 to 38500, 38501 to 39000, 39001 to 39500, 39501 to 40000, 40001 to 40500, 40501 to 41000, 42001 to 42500, 42501 to 43000, 43001 to 43500, 43501 to 44000, 44001 to 44500, 44501 to 45000, 45001 to 45500, 45501 to 46000, 46001 to 46500, 46501 to 47000, 47001 to 47500, 47501 to 48000, 48001 to 48500, 48501 to 49000, 49001 to 49500, 49501 to 50000, 50001 to 50500, 50501 to 51000, 51001 to 51500, 51501 to 52000, 52001 to 52500, 52501 to 53000, 53001 to 53500, 53501 to 54000, 54001 to 54500, 54501 to 55000, 55001 to 55500, 56001 to 56500, 56501 to 57000, 57001 to 57500, 57501 to 58000, 58001 to 58500, 58501 to 59000, 59001 to 59500, 59501 to 60000, 60001 to 60500, 60501 to 61000, 61001 to 61500, 61501 to 62000, 62001 to 62500, 62501 to 63000, 63001 to 63500, 63501 to 64000, 64001 to 64500, 64501 to 65000, 65001 to 65500, 65501 to 66000, 66001 to 66500, 66501 to 67000, 67001 to 67500, 67501 to 68000, 68001 to 68500, 68501 to 69000, 69001 to 69500, 69501 to 70000, 70001 to 70500, 70501 to 71000, 71001 to 71500, 71501 to 72000, 72001 to 72500, 72501 to 73000, 73001 to 73500, 73501 to 74000, 74001 to 74500, 74501 to 75000, 75001 to 75500, 75501 to 76000, 76001 to 76500, 76501 to 77000, 77001 to 77500, 77501 to 78000, 78001 to 78500, 78501 to 79000, 79001 to 79500, 79501 to 80000, 80001 to 80500, 80501 to 81000, 81001 to 81500, 81501 to 82000, 82001 to 82500, 82501 to 83000, 83001 to 83500, 83501 to 84000, 84001 to 84500, 84501 to 85000, 85001 to 85500, 85501 to 86000, 86001 to 86500, 86501 to 87000, 87001 to 87500, 87501 to 88000, 88001 to 88500, 88501 to 89000, 89001 to 89500, 89501 to 90000, 90001 to 90500, 90501 to 91000, 91001 to 91500, 91501 to 92000, 92001 to 92500, 92501 to 93000, 93001 to 93500, 93501 to 94000, 94001 to 94500, 94501 to 95000, 95001 to 95500, 95501 to 96000, 96001 to 96500, 96501 to 97000, 97001 to 97500, 97501 to 98000, 98001 to 98500, 98501 to 99000, 99001 to 99500, 99501 to 100000, 100001 to 100500, 100501 to 101000, 101001 to 101500, 101501 to 102000, 102001 to 102500, 102501 to 103000, 103001 to 103500, 103501 to 104000, 104001 to 104500, 105001 to 105500, 105501 to 106000, 106001 to 106500, 106501 to 107000, 107001 to 107500, 108001 to 108500, 108501 to 109000, 109001 to 109500, 109501 to 110000, 110001 to 110500, 110501 to 111000, 111001 to 111500, 111501 to 112000, 112001 to 112500, 112501 to 113000, 113001 to 113500, 113501 to 114000, 114001 to 114500, 114501 to 115000, 115001 to 115500, 115501 to 116000, 116001 to 116500, 116501 to 117000, 117001 to 117500, 117501 to 118000, 118001 to 118500, 118501 to 119000, 119001 to 119500, 119501 to 120000, 120001 to 120500, 120501 to 121000, 121001 to 121500, 122001 to 122500, 122501 to 123000, 123001 to 123500, 124001 to 124500, 124501 to 125000, 125001 to 126000, 126001 to 126500, 127001 to 127500, 129001 to 129500, 129501 to 130000, 130001 to 130500, 131001 to 131500, 131501 to 132000, 132501 to 133000, 134001 to 134500, 134501 to 135000, 135001 to 135500, 135501 to 136000, 136001 to 136500, 136501 to 137000, 137001 to 137500, 137501 to 138000, 138001 to 138500, 138501 to 139000, 139001 to 139500, 139501 to 140000, 140001 to 140500, 140501 to 141000, 141001 to 141500, 141501 to 142000, 142001 to 142500, 142501 to 143000, 143001 to 143500, 143501 to 144000, or 144001 to 144173 of SEQ ID NO. 2. ASOs hybridizing in these regions are shown in the following Table 2:

| Regionon Seq ID No. 2/ | Position on | |
|---|---|---|
| ASO name | Seq ID No 2 | SEQ ID NO. |
| 1-500 | | |
| A57250H | 228 | 252 |
| A57251H | 229 | 253 |
| A57261H | 56 | 263 |
| A57262H | 57 | 264 |
| A57263H | 57 | 265 |
| A57264H | 59 | 266 |
| A57265H | 62 | 267 |
| A57266H | 63 | 268 |
| A57267H | 63 | 269 |
| A57268H | 64 | 270 |
| A57269H | 64 | 271 |
| A57270H | 65 | 272 |
| A57271H | 67 | 273 |
| A57272H | 68 | 274 |
| A57273H | 68 | 275 |
| A57274H | 69 | 276 |
| A57275H | 70 | 277 |
| A57276H | 71 | 278 |
| A57277H | 71 | 279 |
| A57278H | 72 | 280 |
| A57279H | 72 | 281 |
| A57280H | 73 | 282 |
| A57281H | 74 | 283 |
| A57282H | 74 | 284 |
| A57283H | 75 | 285 |
| A57284H | 75 | 286 |
| A57285H | 75 | 287 |
| A57286H | 76 | 288 |
| A57287H | 77 | 289 |
| A57288H | 78 | 290 |
| A57289H | 78 | 291 |
| A57290H | 79 | 292 |
| A57291H | 79 | 293 |
| A57292H | 79 | 294 |
| A57293H | 80 | 295 |
| A57294H | 80 | 296 |
| A57295H | 80 | 297 |
| A57296H | 81 | 298 |
| A57297H | 105 | 299 |
| A57298H | 106 | 300 |
| A57299H | 106 | 301 |
| A57300H | 107 | 302 |
| A57301H | 107 | 303 |
| A57302H | 108 | 304 |
| A57303H | 108 | 305 |
| A57304H | 110 | 306 |
| A57305H | 111 | 307 |
| A57306H | 131 | 308 |
| A57307H | 133 | 309 |
| A57308H | 134 | 310 |
| A57309H | 134 | 311 |
| A57310H | 136 | 312 |
| A57311H | 138 | 313 |
| A57312H | 140 | 314 |
| A57313H | 142 | 315 |
| A57314H | 166 | 316 |
| A57315H | 167 | 317 |
| A57316H | 167 | 318 |
| A57317H | 168 | 319 |
| A57318H | 168 | 320 |
| A57319H | 168 | 321 |
| A57320H | 169 | 322 |
| A57321H | 170 | 323 |
| A57322H | 170 | 324 |
| A57323H | 171 | 325 |
| A57324H | 171 | 326 |
| A57325H | 180 | 327 |
| A57326H | 185 | 328 |
| A57327H | 186 | 329 |
| A57328H | 186 | 330 |
| A57329H | 187 | 331 |
| A57330H | 187 | 332 |
| A57331H | 188 | 333 |
| A57332H | 188 | 334 |
| A57333H | 189 | 335 |
| A57334H | 189 | 336 |
| A57335H | 189 | 337 |
| A57336H | 190 | 338 |
| A57337H | 190 | 339 |
| A57338H | 191 | 340 |
| A57339H | 191 | 341 |
| A57340H | 193 | 342 |
| A57341H | 194 | 343 |
| A57342H | 195 | 344 |
| A57343H | 199 | 345 |
| A57344H | 200 | 346 |
| A57345H | 225 | 347 |
| A57346H | 226 | 348 |
| A57347H | 226 | 349 |
| A57348H | 227 | 350 |
| A57349H | 228 | 351 |
| A57350H | 229 | 352 |
| A57351H | 229 | 353 |
| A57352H | 230 | 354 |
| A57353H | 230 | 355 |
| A57354H | 230 | 356 |
| A57355H | 231 | 357 |
| A57356H | 232 | 358 |
| A57357H | 232 | 359 |
| A57358H | 233 | 360 |
| A57359H | 233 | 361 |
| A57360H | 233 | 362 |
| A57361H | 234 | 363 |
| A57362H | 234 | 364 |
| A57363H | 235 | 365 |
| A57364H | 239 | 366 |
| A57365H | 240 | 367 |
| A57366H | 240 | 368 |
| A57367H | 241 | 369 |
| A57368H | 241 | 370 |
| A57369H | 248 | 371 |
| A57370H | 251 | 372 |
| A57371H | 253 | 373 |
| A57372H | 254 | 374 |
| A57373H | 256 | 375 |
| A57374H | 257 | 376 |
| A57375H | 258 | 377 |
| A57376H | 259 | 378 |
| A57377H | 260 | 379 |
| A57378H | 261 | 380 |
| A57379H | 262 | 381 |
| A57380H | 263 | 382 |
| A57381H | 263 | 383 |
| A57382H | 264 | 384 |
| A57383H | 264 | 385 |
| A57384H | 264 | 386 |
| A57385H | 265 | 387 |
| A57386H | 265 | 388 |
| A57387H | 265 | 389 |
| A57388H | 266 | 390 |
| A57389H | 266 | 391 |
| A57390H | 266 | 392 |
| A57391H | 267 | 393 |
| A57392Hi | 451 | 394 |
| 501-1000 | | |
| A57001Hi | 509 | 3 |
| A57002Hi | 681 | 4 |
| A57393Hi | 508 | 395 |
| A57394Hi | 516 | 396 |
| A57395H | 560 | 397 |
| A57396H | 562 | 398 |
| A57397H | 576 | 399 |
| A57398H | 577 | 400 |
| A57399H | 587 | 401 |
| A57400H | 588 | 402 |
| A57401H | 588 | 403 |
| A57402H | 589 | 404 |
| A57403H | 589 | 405 |
| A57404H | 590 | 406 |
| A57405H | 591 | 407 |
| A57406H | 591 | 408 |
| A57407H | 592 | 409 |
| A57408H | 592 | 410 |
| A57409H | 593 | 411 |
| A57410H | 594 | 412 |
| A57411H | 594 | 413 |
| A57412H | 595 | 414 |
| A57413H | 595 | 415 |
| A57414H | 596 | 416 |
| A57415H | 597 | 417 |
| A57416H | 597 | 418 |
| A57417H | 598 | 419 |
| A57418H | 598 | 420 |
| A57419H | 598 | 421 |
| A57420H | 599 | 422 |
| A57421H | 618 | 423 |
| A57422H | 618 | 424 |
| A57423H | 618 | 425 |
| A57424H | 618 | 426 |
| A57425H | 619 | 427 |
| A57426H | 619 | 428 |
| A57427H | 619 | 429 |
| A57428H | 620 | 430 |
| A57429H | 620 | 431 |
| A57430H | 621 | 432 |
| A57431H | 621 | 433 |
| A57432H | 621 | 434 |
| A57433H | 622 | 435 |
| A57434H | 622 | 436 |
| A57435H | 622 | 437 |
| A57436H | 623 | 438 |
| A57437H | 623 | 439 |
| A57438H | 624 | 440 |
| A57439H | 624 | 441 |
| A57440H | 624 | 442 |
| A57441H | 625 | 443 |
| A57442H | 625 | 444 |
| A57443H | 625 | 445 |
| A57444H | 626 | 446 |
| A57445H | 626 | 447 |
| A57446H | 626 | 448 |
| A57447H | 627 | 449 |
| A57448H | 627 | 450 |
| A57449H | 628 | 451 |
| A57450Hi | 683 | 452 |
| A57451Hi | 746 | 453 |
| A57452Hi | 747 | 454 |
| A57453Hi | 747 | 455 |
| 1001-1500 | | |
| A57003Hi | 1112 | 5 |
| A57004Hi | 1142 | 6 |
| A57005Hi | 1299 | 7 |
| A57006Hi | 1459 | 8 |
| A57454Hi | 1074 | 456 |
| A57455Hi | 1074 | 457 |
| A57456Hi | 1078 | 458 |
| A57457Hi | 1298 | 459 |
| A57458Hi | 1303 | 460 |
| A57459Hi | 1307 | 461 |
| A57460Hi | 1397 | 462 |
| A57461Hi | 1398 | 463 |
| A57462Hi | 1399 | 464 |
| A57463Hi | 1399 | 465 |
| A57464Hi | 1404 | 466 |
| A57465Hi | 1452 | 467 |
| A57466Hi | 1453 | 468 |
| A57467Hi | 1454 | 469 |
| A57468Hi | 1455 | 470 |
| A57469Hi | 1455 | 471 |
| A57470Hi | 1456 | 472 |
| A57471Hi | 1465 | 473 |
| A57472Hi | 1466 | 474 |
| 1501-2000 | | |
| A57007Hi | 1718 | 9 |
| A57008Hi | 1720 | 10 |
| A57009Hi | 1749 | 11 |
| A57010Hi | 1773 | 12 |
| A57473Hi | 1719 | 475 |
| A57474Hi | 1723 | 476 |
| A57475Hi | 1726 | 477 |
| A57476Hi | 1748 | 478 |
| A57477Hi | 1749 | 479 |
| A57478Hi | 1753 | 480 |
| A57479Hi | 1754 | 481 |
| A57480Hi | 1803 | 482 |
| A57481Hi | 1806 | 483 |
| A57482Hi | 1807 | 484 |
| A57483Hi | 1808 | 485 |
| A57484Hi | 1812 | 486 |
| 4862001-2500 | | |
| A57485Hi | 2065 | 487 |
| 3501-4000 | 3912 | 488 |
| A57486Hi | | |
| A57487Hi | 3917 | 489 |
| A57488Hi | 3918 | 490 |
| A57489Hi | 3967 | 491 |
| A57490Hi | 3973 | 492 |
| A57491Hi | 3977 | 493 |
| A57492Hi | 3978 | 494 |
| A57493Hi | 3980 | 495 |
| A57494Hi | 3980 | 496 |
| A57495Hi | 3982 | 497 |
| A57496Hi | 3983 | 498 |
| 4001-4500 | | |
| A57011Hi | 4070 | 13 |
| A57012Hi | 4195 | 14 |
| A57497Hi | 4070 | 499 |
| A57498Hi | 4193 | 500 |
| A57499Hi | 4201 | 501 |
| A57500Hi | 4346 | 502 |
| A57501Hi | 4355 | 503 |
| A57502Hi | 4358 | 504 |
| A57503Hi | 4366 | 505 |
| A57504Hi | 4368 | 506 |
| A57505Hi | 4440 | 507 |
| 4501-5000 | | |
| A57013Hi | 4829 | 15 |
| A57014Hi | 4939 | 16 |
| A57015Hi | 4940 | 17 |
| A57506Hi | 4512 | 508 |
| A57507Hi | 4636 | 509 |
| A57508Hi | 4682 | 510 |
| A57509Hi | 4683 | 511 |
| A57510Hi | 4684 | 512 |
| A57511Hi | 4757 | 513 |
| A57512Hi | 4770 | 514 |
| A57513Hi | 4806 | 515 |
| A57514Hi | 4828 | 516 |
| A57515Hi | 4937 | 517 |
| A57516Hi | 4941 | 518 |
| A57517Hi | 4989 | 519 |
| A57518Hi | 4990 | 520 |
| A57519Hi | 4991 | 521 |
| A57520Hi | 4994 | 522 |
| 5001-5500 | | |
| A57016Hi | 5077 | 18 |
| A57017Hi | 5101 | 19 |
| A57018Hi | 5104 | 20 |
| A57019Hi | 5110 | 21 |
| A57020Hi | 5140 | 22 |
| A57240Hi | 5111 | 242 |
| A57521Hi | 5075 | 523 |
| A57522Hi | 5077 | 524 |
| A57523Hi | 5078 | 525 |
| A57524Hi | 5102 | 526 |
| A57525Hi | 5105 | 527 |
| A57526Hi | 5105 | 528 |
| A57527Hi | 5105 | 529 |
| A57528Hi | 5106 | 530 |
| A57529Hi | 5107 | 531 |
| A57530Hi | 5107 | 532 |
| A57531Hi | 5107 | 533 |
| A57532Hi | 5111 | 534 |
| A57533Hi | 5114 | 535 |
| A57534Hi | 5115 | 536 |
| A57535Hi | 5115 | 537 |
| A57536Hi | 5116 | 538 |
| A57537Hi | 5117 | 539 |
| A57538Hi | 5331 | 540 |
| A57539Hi | 5341 | 541 |
| A57540Hi | 5375 | 542 |
| A57541Hi | 5380 | 543 |
| A57542Hi | 5404 | 544 |
| A57543Hi | 5405 | 545 |
| A57544Hi | 5414 | 546 |
| A57545Hi | 5469 | 547 |
| A57546Hi | 5471 | 548 |
| A57547Hi | 5472 | 549 |
| A57548Hi | 5473 | 550 |
| 5501-6000 | | |
| A57021Hi | 5827 | 23 |
| A57022Hi | 5870 | 24 |
| A57023Hi | 5996 | 25 |
| A57549Hi | 5547 | 551 |
| A57550Hi | 5552 | 552 |
| A57551Hi | 5554 | 553 |
| A57552Hi | 5577 | 554 |
| A57553Hi | 5578 | 555 |
| A57554Hi | 5608 | 556 |
| A57555Hi | 5610 | 557 |
| A57556Hi | 5689 | 558 |
| A57557Hi | 5690 | 559 |
| A57558Hi | 5690 | 560 |
| A57559Hi | 5691 | 561 |
| A57560Hi | 5692 | 562 |
| A57561Hi | 5695 | 563 |
| A57562Hi | 5696 | 564 |
| A57563Hi | 5704 | 565 |
| A57564Hi | 5801 | 566 |
| A57565Hi | 5849 | 567 |
| A57566Hi | 5849 | 568 |
| A57567Hi | 5850 | 569 |
| A57568Hi | 5858 | 570 |
| A57569Hi | 5859 | 571 |
| A57570Hi | 5863 | 572 |
| A57571Hi | 5865 | 573 |
| A57572Hi | 5868 | 574 |
| A57573Hi | 5914 | 575 |
| A57574Hi | 5917 | 576 |
| A57575Hi | 5923 | 577 |
| A57576Hi | 5924 | 578 |
| A57577Hi | 5925 | 579 |
| A57578Hi | 5925 | 580 |
| A57579Hi | 5926 | 581 |
| A57580Hi | 5926 | 582 |
| A57581Hi | 5954 | 583 |
| A57582Hi | 5967 | 584 |
| A57583Hi | 5997 | 585 |
| A57584Hi | 5997 | 586 |
| A57585Hi | 5999 | 587 |
| 6001-6500 | | |
| A57586Hi | 6001 | 588 |
| A57587Hi | 6007 | 589 |
| A57588Hi | 6085 | 590 |
| A57589Hi | 6095 | 591 |
| A57590Hi | 6096 | 592 |
| A57591Hi | 6127 | 593 |
| A57592Hi | 6129 | 594 |
| A57593Hi | 6130 | 595 |
| 6501-7000 | | |
| A57024Hi | 6644 | 26 |
| A57594Hi | 6639 | 596 |
| A57595Hi | 6643 | 597 |
| A57596Hi | 6644 | 598 |
| A57597Hi | 6650 | 599 |
| 7001-7500 | | |
| A57598Hi | 7079 | 600 |
| A57599Hi | 7080 | 601 |
| A57600Hi | 7201 | 602 |
| A57601H | 7282 | 603 |
| A57602H | 7290 | 604 |
| A57603H | 7320 | 605 |
| A57604H | 7321 | 606 |
| A57605H | 7324 | 607 |
| A57606H | 7325 | 608 |
| A57607H | 7326 | 609 |
| A57608H | 7327 | 610 |
| A57609H | 7328 | 611 |
| A57610H | 7329 | 612 |
| A57611H | 7329 | 613 |
| A57612H | 7334 | 614 |
| A57613H | 7339 | 615 |
| A57614H | 7345 | 616 |
| A57615H | 7346 | 617 |
| A57616H | 7349 | 618 |
| A57617H | 7350 | 619 |
| A57618H | 7352 | 620 |
| A57619H | 7353 | 621 |
| A57620H | 7355 | 622 |
| A57621H | 7356 | 623 |
| A57622H | 7357 | 624 |
| A57623H | 7358 | 625 |
| A57624H | 7365 | 626 |
| A57625H | 7366 | 627 |
| A57626Hi | 7390 | 628 |
| A57627Hi | 7467 | 629 |
| A57628Hi | 7467 | 630 |
| A57629Hi | 7468 | 631 |
| A57630Hi | 7468 | 632 |
| 7501-8000 | | |
| A57025Hi | 7773 | 27 |
| A57026Hi | 7944 | 28 |
| A57631Hi | 7516 | 633 |
| A57632Hi | 7528 | 634 |
| A57633Hi | 7540 | 635 |
| A57634Hi | 7543 | 636 |
| A57635Hi | 7554 | 637 |
| A57636Hi | 7556 | 638 |
| A57637Hi | 7560 | 639 |
| A57638Hi | 7583 | 640 |
| A57639Hi | 7584 | 641 |
| A57640Hi | 7774 | 642 |
| A57641Hi | 7775 | 643 |
| A57642Hi | 7945 | 644 |
| A57643Hi | 7946 | 645 |
| A57644Hi | 7974 | 646 |
| A57645Hi | 7975 | 647 |
| A57646Hi | 7976 | 648 |
| 8001-8500 | | |
| A57647Hi | 8480 | 649 |
| A57648Hi | 8481 | 650 |
| 8501-9000 | | |
| A57649Hi | 8530 | 651 |
| A57650Hi | 8534 | 652 |
| A57651Hi | 8619 | 653 |
| A57652Hi | 8705 | 654 |
| A57653Hi | 8707 | 655 |
| A57654Hi | 8713 | 656 |
| A57655Hi | 8714 | 657 |
| A57656Hi | 8716 | 658 |
| A57657Hi | 8748 | 659 |
| A57658Hi | 8749 | 660 |
| A57659Hi | 8749 | 661 |
| A57660Hi | 8751 | 662 |
| A57661Hi | 8843 | 663 |
| A57662Hi | 8922 | 664 |
| 9001-9500 | | |
| A57027Hi | 9182 | 29 |
| A57028Hi | 9185 | 30 |
| A57663Hi | 9181 | 665 |
| A57664Hi | 9278 | 666 |
| 9501-10000 | | |
| A57029Hi | 9717 | 31 |
| A57665Hi | 9718 | 667 |
| A57666Hi | 9719 | 668 |
| A57667Hi | 9761 | 669 |
| A57668Hi | 9763 | 670 |
| A57669Hi | 9764 | 671 |
| A57670Hi | 9765 | 672 |
| A57671Hi | 9765 | 673 |
| A57672Hi | 9767 | 674 |
| A57673Hi | 9804 | 675 |
| A57674Hi | 9807 | 676 |
| A57675Hi | 9808 | 677 |
| A57676Hi | 9808 | 678 |
| 10501-11000 | | |
| A57677Hi | 10519 | 679 |
| A57678H | 10642 | 680 |
| A57679H | 10643 | 681 |
| A57680H | 10644 | 682 |
| A57681H | 10644 | 683 |
| A57682H | 10645 | 684 |
| A57683H | 10645 | 685 |
| A57684H | 10646 | 686 |
| A57685H | 10646 | 687 |
| A57686H | 10649 | 688 |
| A57687H | 10651 | 689 |
| A57688H | 10652 | 690 |
| A57689H | 10659 | 691 |
| A57690H | 10660 | 692 |
| A57691H | 10660 | 693 |
| A57692H | 10661 | 694 |
| A57693H | 10680 | 695 |
| A57694H | 10680 | 696 |
| A57695H | 10681 | 697 |
| A57696H | 10682 | 698 |
| A57697H | 10685 | 699 |
| A57698H | 10687 | 700 |
| A57699H | 10688 | 701 |
| A57700H | 10689 | 702 |
| A57701H | 10690 | 703 |
| A57702H | 10691 | 704 |
| A57703H | 10692 | 705 |
| A57704H | 10693 | 706 |
| A57705Hi | 10837 | 707 |
| 11001-11500 | | |
| A57030Hi | 11171 | 32 |
| A57031Hi | 11174 | 33 |
| A57706Hi | 11171 | 708 |
| A57707Hi | 11173 | 709 |
| A57708Hi | 11233 | 710 |
| A57709Hi | 11234 | 711 |
| A57710Hi | 11234 | 712 |
| A57711Hi | 11237 | 713 |
| A57712Hi | 11238 | 714 |
| A57713Hi | 11238 | 715 |
| A57714Hi | 11239 | 716 |
| A57715Hi | 11239 | 717 |
| A57716Hi | 11240 | 718 |
| A57717Hi | 11241 | 719 |
| A57718Hi | 11242 | 720 |
| A57719Hi | 11393 | 721 |
| A57720Hi | 11396 | 722 |
| A57721Hi | 11397 | 723 |
| A57722Hi | 11397 | 724 |
| A57723Hi | 11398 | 725 |
| A57724Hi | 11460 | 726 |
| A57725Hi | 11489 | 727 |
| 11501-12000 | | |
| A57032Hi | 11731 | 34 |
| A57726Hi | 11573 | 728 |
| A57727Hi | 11689 | 729 |
| A57728Hi | 11689 | 730 |
| A57729Hi | 11691 | 731 |
| A57730Hi | 11732 | 732 |
| A57731Hi | 11733 | 733 |
| A57732Hi | 11959 | 734 |
| A57733Hi | 11959 | 735 |
| A57734Hi | 11960 | 736 |
| A57735Hi | 11962 | 737 |
| 12001-12500 | | |
| A57033Hi | 12157 | 35 |
| A57034Hi | 12449 | 36 |
| A57035Hi | 12455 | 37 |
| A57736Hi | 12012 | 738 |
| A57737Hi | 12146 | 739 |
| A57738Hi | 12148 | 740 |
| A57739Hi | 12152 | 741 |
| A57740Hi | 12153 | 742 |
| A57741Hi | 12189 | 743 |
| A57742Hi | 12191 | 744 |
| A57743Hi | 12193 | 745 |
| A57744Hi | 12194 | 746 |
| A57745Hi | 12382 | 747 |
| A57746Hi | 12383 | 748 |
| A57747Hi | 12404 | 749 |
| A57748Hi | 12408 | 750 |
| A57749Hi | 12409 | 751 |
| A57750Hi | 12410 | 752 |
| A57751Hi | 12411 | 753 |
| A57752Hi | 12414 | 754 |
| A57753Hi | 12457 | 755 |
| 12501-13000 | | |
| A57036Hi | 12590 | 38 |
| A57037Hi | 12652 | 39 |
| A57038Hi | 12668 | 40 |
| A57754Hi | 12586 | 756 |
| A57755Hi | 12587 | 757 |
| A57756Hi | 12589 | 758 |
| A57757Hi | 12653 | 759 |
| A57758Hi | 12654 | 760 |
| A57759Hi | 12655 | 761 |
| A57760Hi | 12655 | 762 |
| A57761Hi | 12660 | 763 |
| A57762Hi | 12661 | 764 |
| A57763Hi | 12669 | 765 |
| A57764Hi | 12770 | 766 |
| A57765Hi | 12773 | 767 |
| A57766Hi | 12774 | 768 |
| A57767Hi | 12779 | 769 |
| A57768Hi | 12785 | 770 |
| 13001-13500 | | |
| A57769H | 13017 | 771 |
| A57770H | 13019 | 772 |
| A57771H | 13022 | 773 |
| A57772H | 13023 | 774 |
| A57773H | 13023 | 775 |
| A57774H | 13027 | 776 |
| A57775H | 13030 | 777 |
| A57776H | 13031 | 778 |
| A57777H | 13032 | 779 |
| A57778H | 13032 | 780 |
| A57779H | 13036 | 781 |
| A57780H | 13037 | 782 |
| A57781H | 13039 | 783 |
| A57782H | 13040 | 784 |
| A57783H | 13041 | 785 |
| A57784H | 13041 | 786 |
| A57785H | 13042 | 787 |
| A57786H | 13043 | 788 |
| A57787H | 13064 | 789 |
| A57788H | 13068 | 790 |
| A57789H | 13071 | 791 |
| A57790H | 13072 | 792 |
| A57791H | 13072 | 793 |
| A57792Hi | 13349 | 794 |
| A57793Hi | 13350 | 795 |
| A57794Hi | 13350 | 796 |
| A57795Hi | 13351 | 797 |
| A57796Hi | 13351 | 798 |
| A57797Hi | 13351 | 799 |
| A57798Hi | 13414 | 800 |
| A57799Hi | 13414 | 801 |
| A57800Hi | 13417 | 802 |
| A57801Hi | 13417 | 803 |
| A57802Hi | 13438 | 804 |
| 13501-14000 | | |
| A57039Hi | 13583 | 41 |
| A57040Hi | 13591 | 42 |
| A57041Hi | 13785 | 43 |
| A57803Hi | 13591 | 805 |
| A57804Hi | 13601 | 806 |
| A57805Hi | 13602 | 807 |
| A57806Hi | 13636 | 808 |
| A57807Hi | 13641 | 809 |
| A57808Hi | 13642 | 810 |
| A57809Hi | 13674 | 811 |
| A57810Hi | 13675 | 812 |
| A57811Hi | 13677 | 813 |
| A57812Hi | 13679 | 814 |
| A57813Hi | 13680 | 815 |
| A57814Hi | 13682 | 816 |
| A57815Hi | 13786 | 817 |
| 14501-15000 | | |
| A57816Hi | 14813 | 818 |
| A57817Hi | 14861 | 819 |
| A57818Hi | 14931 | 820 |
| A57819Hi | 14934 | 821 |
| A57820Hi | 14935 | 822 |
| A57821Hi | 14939 | 823 |
| A57822Hi | 14939 | 824 |
| A57823Hi | 14940 | 825 |
| A57824Hi | 14944 | 826 |
| A57825Hi | 14945 | 827 |
| 15001-15500 | | |
| A57042Hi | 15055 | 44 |
| A57043Hi | 15065 | 45 |
| A57044Hi | 15067 | 46 |
| A57826Hi | 15029 | 828 |
| A57827Hi | 15031 | 829 |
| A57828Hi | 15035 | 830 |
| A57829Hi | 15035 | 831 |
| A57830Hi | 15036 | 832 |
| A57831Hi | 15040 | 833 |
| A57832Hi | 15040 | 834 |
| A57833Hi | 15043 | 835 |
| A57834Hi | 15045 | 836 |
| A57835Hi | 15054 | 837 |
| A57836Hi | 15056 | 838 |
| A57837Hi | 15066 | 839 |
| A57838Hi | 15066 | 840 |
| A57839Hi | 15067 | 841 |
| A57840Hi | 15067 | 842 |
| A57841Hi | 15068 | 843 |
| A57842Hi | 15068 | 844 |
| A57843Hi | 15096 | 845 |
| A57844Hi | 15099 | 846 |
| A57845Hi | 15153 | 847 |
| A57846Hi | 15156 | 848 |
| 15501-16000 | | |
| A57045Hi | 15852 | 47 |
| A57847H | 15509 | 849 |
| A57848H | 15510 | 850 |
| A57849H | 15511 | 851 |
| A57850H | 15511 | 852 |
| A57851H | 15512 | 853 |
| A57852H | 15513 | 854 |
| A57853H | 15515 | 855 |
| A57854H | 15525 | 856 |
| A57855H | 15529 | 857 |
| A57856H | 15530 | 858 |
| A57857H | 15531 | 859 |
| A57858H | 15531 | 860 |
| A57859H | 15534 | 861 |
| A57860H | 15535 | 862 |
| A57861H | 15535 | 863 |
| A57862H | 15536 | 864 |
| A57863H | 15536 | 865 |
| A57864H | 15536 | 866 |
| A57865H | 15538 | 867 |
| A57866H | 15539 | 868 |
| A57867H | 15539 | 869 |
| A57868H | 15541 | 870 |
| A57869H | 15544 | 871 |
| A57870H | 15569 | 872 |
| A57871H | 15571 | 873 |
| A57872H | 15592 | 874 |
| A57873H | 15595 | 875 |
| A57874Hi | 15781 | 876 |
| A57875Hi | 15851 | 877 |
| A57876Hi | 15852 | 878 |
| 16001-16500 | | |
| A57046Hi | 16213 | 48 |
| A57877Hi | 16085 | 879 |
| A57878Hi | 16086 | 880 |
| A57879Hi | 16086 | 881 |
| A57880Hi | 16087 | 882 |
| A57881Hi | 16091 | 883 |
| A57882Hi | 16094 | 884 |
| A57883Hi | 16095 | 885 |
| A57884Hi | 16095 | 886 |
| A57885Hi | 16097 | 887 |
| A57886Hi | 16098 | 888 |
| A57887Hi | 16098 | 889 |
| A57888Hi | 16099 | 890 |
| A57889Hi | 16211 | 891 |
| A57890Hi | 16212 | 892 |
| A57891Hi | 16261 | 893 |
| A57892Hi | 16262 | 894 |
| A57893Hi | 16271 | 895 |
| 16501-17000 | | |
| A57047Hi | 16735 | 49 |
| A57894Hi | 16548 | 896 |
| A57895Hi | 16558 | 897 |
| A57896Hi | 16588 | 898 |
| A57897Hi | 16661 | 899 |
| A57898Hi | 16663 | 900 |
| A57899Hi | 16707 | 901 |
| A57900Hi | 16741 | 902 |
| A57901Hi | 16857 | 903 |
| A57902Hi | 16895 | 904 |
| A57903Hi | 16901 | 905 |
| A57904Hi | 16902 | 906 |
| 17001-17500 | | |
| A57905Hi | 17181 | 907 |
| A57906Hi | 17181 | 908 |
| A57907Hi | 17355 | 909 |
| 17501-18000 | | |
| A57908Hi | 17548 | 910 |
| A57909Hi | 17716 | 911 |
| A57910Hi | 17716 | 912 |
| A57911Hi | 17717 | 913 |
| 18001-18500 | | |
| A57048Hi | 18310 | 50 |
| A57912Hi | 18299 | 914 |
| A57913Hi | 18300 | 915 |
| A57914Hi | 18301 | 916 |
| A57915Hi | 18308 | 917 |
| A57916Hi | 18421 | 918 |
| A57917Hi | 18422 | 919 |
| A57918Hi | 18447 | 920 |
| A57919Hi | 18449 | 921 |
| A57920Hi | 18451 | 922 |
| 18501-19000 | | |
| A57049Hi | 18767 | 51 |
| A57921Hi | 18521 | 923 |
| A57922H | 18559 | 924 |
| A57923H | 18560 | 925 |
| A57924H | 18561 | 926 |
| A57925H | 18561 | 927 |
| A57926H | 18564 | 928 |
| A57927H | 18567 | 929 |
| A57928H | 18568 | 930 |
| A57929H | 18571 | 931 |
| A57930H | 18572 | 932 |
| A57931H | 18573 | 933 |
| A57932H | 18574 | 934 |
| A57933H | 18576 | 935 |
| A57934H | 18577 | 936 |
| A57935H | 18578 | 937 |
| A57936H | 18579 | 938 |
| A57937H | 18582 | 939 |
| A57938H | 18588 | 940 |
| A57939H | 18593 | 941 |
| A57940H | 18595 | 942 |
| A57941H | 18596 | 943 |
| A57942H | 18596 | 944 |
| A57943H | 18597 | 945 |
| A57944H | 18597 | 946 |
| A57945H | 18600 | 947 |
| A57946H | 18601 | 948 |
| A57947H | 18602 | 949 |
| A57948H | 18608 | 950 |
| A57949H | 18609 | 951 |
| A57950H | 18610 | 952 |
| A57951H | 18612 | 953 |
| A57952H | 18649 | 954 |
| A57953H | 18650 | 955 |
| A57954H | 18650 | 956 |
| A57955H | 18652 | 957 |
| A57956H | 18656 | 958 |
| A57957H | 18661 | 959 |
| A57958H | 18662 | 960 |
| A57959H | 18662 | 961 |
| A57960H | 18666 | 962 |
| A57961H | 18667 | 963 |
| A57962Hi | 18769 | 964 |
| A57963Hi | 18771 | 965 |
| 19001-19500 | | |
| A57964Hi | 19145 | 966 |
| A57965Hi | 19145 | 967 |
| A57966Hi | 19146 | 968 |
| A57967Hi | 19147 | 969 |
| A57968Hi | 19151 | 970 |
| A57969Hi | 19152 | 971 |
| A57970Hi | 19442 | 972 |
| 20001-20500 | | |
| A57050Hi | 20304 | 52 |
| A57971Hi | 20096 | 973 |
| A57972Hi | 20266 | 974 |
| A57973Hi | 20271 | 975 |
| A57974Hi | 20273 | 976 |
| A57975Hi | 20296 | 977 |
| A57976Hi | 20303 | 978 |
| A57977Hi | 20386 | 979 |
| A57978Hi | 20387 | 980 |
| A57979Hi | 20391 | 981 |
| 20501-21000 | | |
| A57051Hi | 20604 | 53 |
| A57052Hi | 20895 | 54 |
| A57053Hi | 20900 | 55 |
| A57980Hi | 20509 | 982 |
| A57981Hi | 20510 | 983 |
| A57982Hi | 20510 | 984 |
| A57983Hi | 20812 | 985 |
| A57984Hi | 20899 | 986 |
| A57985Hi | 20900 | 987 |
| 21001-21500 | | |
| A57986Hi | 21288 | 988 |
| A57987Hi | 21292 | 989 |
| A57988Hi | 21330 | 990 |
| A57989Hi | 21331 | 991 |
| A57990Hi | 21495 | 992 |
| 21501-22000 | | |
| A57054Hi | 21645 | 56 |
| A57055Hi | 21650 | 57 |
| A571000Hi | 21566 | 1002 |
| A571001Hi | 21640 | 1003 |
| A571002Hi | 21641 | 1004 |
| A571003Hi | 21644 | 1005 |
| A571004Hi | 21647 | 1006 |
| A571005Hi | 21649 | 1007 |
| A571006Hi | 21650 | 1008 |
| A571007Hi | 21658 | 1009 |
| A571008Hi | 21689 | 1010 |
| A571009Hi | 21763 | 1011 |
| A571010Hi | 21767 | 1012 |
| A571011Hi | 21839 | 1013 |
| A571012Hi | 21840 | 1014 |
| A571013Hi | 21845 | 1015 |
| A571014Hi | 21845 | 1016 |
| A571015Hi | 21924 | 1017 |
| A57991Hi | 21551 | 993 |
| A57992Hi | 21551 | 994 |
| A57993Hi | 21554 | 995 |
| A57994Hi | 21555 | 996 |
| A57995Hi | 21556 | 997 |
| A57996Hi | 21557 | 998 |
| A57997Hi | 21562 | 999 |
| A57998Hi | 21563 | 1000 |
| A57999Hi | 21563 | 1001 |
| 22001-22500 | | |
| A57056Hi | 22023 | 58 |
| A571016Hi | 22024 | 1018 |
| A571017Hi | 22053 | 1019 |
| A571018Hi | 22170 | 1020 |
| A571019Hi | 22171 | 1021 |
| 23001-23500 | | |
| A571020Hi | 23204 | 1022 |
| A571021Hi | 23205 | 1023 |
| A571022Hi | 23207 | 1024 |
| A571023Hi | 23207 | 1025 |
| A571024Hi | 23218 | 1026 |
| A571025Hi | 23306 | 1027 |
| A571026Hi | 23308 | 1028 |
| A571027Hi | 23310 | 1029 |
| 23501-24000 | | |
| A57057Hi | 23835 | 59 |
| A571028Hi | 23833 | 1030 |
| A571029Hi | 23834 | 1031 |
| A571030Hi | 23836 | 1032 |
| A571031Hi | 23836 | 1033 |
| A571032Hi | 23951 | 1034 |
| A571033Hi | 23952 | 1035 |
| A571034Hi | 23952 | 1036 |
| A571035Hi | 23953 | 1037 |
| A571036Hi | 23953 | 1038 |
| A571037Hi | 23954 | 1039 |
| A571038Hi | 23955 | 1040 |
| A571039Hi | 23955 | 1041 |
| A571040Hi | 23956 | 1042 |
| A571041Hi | 23956 | 1043 |
| A571042Hi | 23991 | 1044 |
| A571043Hi | 23993 | 1045 |
| A571044Hi | 23994 | 1046 |
| A571045Hi | 23996 | 1047 |
| A571046Hi | 23997 | 1048 |
| A571047Hi | 23999 | 1049 |
| A571048Hi | 24000 | 1050 |
| 24001-24500 | | |
| A57058Hi | 24080 | 60 |
| A57059Hi | 24081 | 61 |
| A57060Hi | 24338 | 62 |
| A571049Hi | 24001 | 1051 |
| A571050Hi | 24001 | 1052 |
| A571051Hi | 24071 | 1053 |
| A571052Hi | 24074 | 1054 |
| A571053Hi | 24080 | 1055 |
| A571054Hi | 24087 | 1056 |
| A571055Hi | 24087 | 1057 |
| A571056Hi | 24217 | 1058 |
| A571057Hi | 24245 | 1059 |
| A571058Hi | 24302 | 1060 |
| A571059Hi | 24336 | 1061 |
| A571060Hi | 24337 | 1062 |
| A571061Hi | 24342 | 1063 |
| A571062Hi | 24395 | 1064 |
| 24501-25000 | | |
| A57061Hi | 24615 | 63 |
| A57062Hi | 24618 | 64 |
| A571063Hi | 24768 | 1065 |
| A571064Hi | 24779 | 1066 |
| A571065H | 24841 | 1067 |
| A571066H | 24842 | 1068 |
| A571067H | 24842 | 1069 |
| A571068H | 24843 | 1070 |
| A571069H | 24843 | 1071 |
| A571070H | 24843 | 1072 |
| A571071H | 24844 | 1073 |
| A571072H | 24846 | 1074 |
| A571073H | 24848 | 1075 |
| A571074H | 24848 | 1076 |
| A571075H | 24849 | 1077 |
| A571076H | 24849 | 1078 |
| A571077H | 24850 | 1079 |
| A571078H | 24851 | 1080 |
| A571079H | 24851 | 1081 |
| A571080H | 24852 | 1082 |
| A571081H | 24871 | 1083 |
| A571082H | 24922 | 1084 |
| A571083H | 24923 | 1085 |
| A571084H | 24923 | 1086 |
| A571085H | 24925 | 1087 |
| A571086H | 24926 | 1088 |
| A571087H | 24926 | 1089 |
| A57252H | 24847 | 254 |
| A57253H | 24847 | 255 |
| A57254H | 24849 | 256 |
| 25001-25500 | | |
| A571088Hi | 25007 | 1090 |
| A571089Hi | 25008 | 1091 |
| A571090Hi | 25009 | 1092 |
| A571091Hi | 25009 | 1093 |
| A571092Hi | 25010 | 1094 |
| A571093Hi | 25012 | 1095 |
| A571094Hi | 25014 | 1096 |
| A571095Hi | 25020 | 1097 |
| A571096Hi | 25143 | 1098 |
| A571097Hi | 25156 | 1099 |
| A571098Hi | 25166 | 1100 |
| 26001-26500 | | |
| A571099Hi | 26097 | 1101 |
| A571100Hi | 26122 | 1102 |
| A571101Hi | 26122 | 1103 |
| A571102Hi | 26125 | 1104 |
| A571103Hi | 26126 | 1105 |
| A571104Hi | 26129 | 1106 |
| A571105Hi | 26166 | 1107 |
| A571106Hi | 26170 | 1108 |
| A571107Hi | 26171 | 1109 |
| A571108H | 26250 | 1110 |
| A571109H | 26280 | 1111 |
| A571110H | 26282 | 1112 |
| A571111H | 26318 | 1113 |
| A571112H | 26320 | 1114 |
| A571113H | 26323 | 1115 |
| A571114H | 26325 | 1116 |
| A571115H | 26326 | 1117 |
| A571116H | 26346 | 1118 |
| A571117H | 26347 | 1119 |
| A571118H | 26347 | 1120 |
| A571119H | 26348 | 1121 |
| A571120H | 26348 | 1122 |
| A571121H | 26349 | 1123 |
| A571122H | 26350 | 1124 |
| A571123H | 26350 | 1125 |
| A571124H | 26355 | 1126 |
| A571125H | 26357 | 1127 |
| A571126H | 26358 | 1128 |
| A571127H | 26358 | 1129 |
| A571128H | 26359 | 1130 |
| A571129H | 26359 | 1131 |
| A571130H | 26359 | 1132 |
| A571131Hi | 26386 | 1133 |
| A571132H | 26477 | 1134 |
| A571133H | 26478 | 1135 |
| A571134H | 26479 | 1136 |
| A571135H | 26479 | 1137 |
| A571136H | 26482 | 1138 |
| A571137H | 26483 | 1139 |
| A571138H | 26487 | 1140 |
| A571139H | 26490 | 1141 |
| A571140H | 26493 | 1142 |
| A571141H | 26494 | 1143 |
| A571142H | 26495 | 1144 |
| A571143H | 26496 | 1145 |
| A571144H | 26497 | 1146 |
| A571145H | 26499 | 1147 |
| 26501-27000 | | |
| A57063Hi | 26745 | 65 |
| A57064Hi | 26913 | 66 |
| A57065Hi | 26978 | 67 |
| A571146H | 26503 | 1148 |
| A571147H | 26505 | 1149 |
| A571148H | 26514 | 1150 |
| A571149H | 26515 | 1151 |
| A571150H | 26516 | 1152 |
| A571151H | 26517 | 1153 |
| A571152H | 26520 | 1154 |
| A571153H | 26521 | 1155 |
| A571154H | 26530 | 1156 |
| A571155Hi | 26746 | 1157 |
| A571156Hi | 26749 | 1158 |
| A571157Hi | 26861 | 1159 |
| A571158Hi | 26865 | 1160 |
| A571159Hi | 26902 | 1161 |
| A571160Hi | 26903 | 1162 |
| A571161Hi | 26908 | 1163 |
| A571162Hi | 26909 | 1164 |
| A571163Hi | 26910 | 1165 |
| A571164Hi | 26911 | 1166 |
| A571165Hi | 26912 | 1167 |
| A571166Hi | 26912 | 1168 |
| A571167Hi | 26914 | 1169 |
| A571168Hi | 26975 | 1170 |
| A571169Hi | 26976 | 1171 |
| A571170Hi | 26978 | 1172 |
| A571171Hi | 26979 | 1173 |
| A571172Hi | 26980 | 1174 |
| A571173Hi | 26984 | 1175 |
| A571174Hi | 26984 | 1176 |
| A571175Hi | 26986 | 1177 |
| 27001-27500 | | |
| A57066Hi | 27011 | 68 |
| A57067Hi | 27238 | 69 |
| A575301Hi | 27238 | 69 |
| A575302Hi | 27238 | 69 |
| A575304Hi | 27238 | 69 |
| A575307Hi | 27238 | 69 |
| A575308Hi | 27238 | 69 |
| A575309Hi | 27238 | 69 |
| A575310Hi | 27238 | 69 |
| A575312Hi | 27238 | 69 |
| A575313Hi | 27238 | 69 |
| A57068Hi | 27406 | 70 |
| A571176Hi | 27012 | 1178 |
| A571177Hi | 27053 | 1179 |
| A571178Hi | 27055 | 1180 |
| A571179Hi | 27057 | 1181 |
| A571180Hi | 27058 | 1182 |
| A571181Hi | 27121 | 1183 |
| A571182Hi | 27122 | 1184 |
| A571183Hi | 27239 | 1185 |
| A571184Hi | 27383 | 1186 |
| A571185Hi | 27383 | 1187 |
| A571186Hi | 27385 | 1188 |
| A571187Hi | 27386 | 1189 |
| A571188Hi | 27387 | 1190 |
| A571189Hi | 27387 | 1191 |
| A571190Hi | 27388 | 1192 |
| A571191Hi | 27388 | 1193 |
| A571192Hi | 27395 | 1194 |
| A571193Hi | 27405 | 1195 |
| 27501-28000 | | |
| A571194Hi | 27646 | 1196 |
| A571195Hi | 27650 | 1197 |
| A571196Hi | 27651 | 1198 |
| A571197Hi | 27819 | 1199 |
| A571198H | 27917 | 1200 |
| A571199H | 27918 | 1201 |
| A571200H | 27919 | 1202 |
| A571201H | 27919 | 1203 |
| A571202H | 27920 | 1204 |
| A571203H | 27920 | 1205 |
| A571204H | 27921 | 1206 |
| A571205H | 27922 | 1207 |
| A571206H | 27922 | 1208 |
| A571207H | 27960 | 1209 |
| A571208H | 27961 | 1210 |
| A571209H | 27961 | 1211 |
| 28001-28500 | | |
| A571210Hi | 28236 | 1212 |
| A571211Hi | 28237 | 1213 |
| 29001-29500 | | |
| A57069Hi | 29066 | 71 |
| A571212Hi | 29068 | 1214 |
| A571213Hi | 29069 | 1215 |
| A571214Hi | 29069 | 1216 |
| A571215Hi | 29073 | 1217 |
| A571216Hi | 29113 | 1218 |
| A571217Hi | 29134 | 1219 |
| A571218Hi | 29214 | 1220 |
| A571219Hi | 29285 | 1221 |
| A571220Hi | 29286 | 1222 |
| A571221Hi | 29434 | 1223 |
| A571222Hi | 29434 | 1224 |
| A571223Hi | 29435 | 1225 |
| A571224Hi | 29435 | 1226 |
| 29501-30000 | | |
| A571225Hi | 29502 | 1227 |
| A571226Hi | 29604 | 1228 |
| A571227Hi | 29607 | 1229 |
| A571228Hi | 29640 | 1230 |
| A571229Hi | 29641 | 1231 |
| A571230Hi | 29645 | 1232 |
| A571231Hi | 29649 | 1233 |
| A571232Hi | 29691 | 1234 |
| A571233Hi | 29691 | 1235 |
| A571234Hi | 29695 | 1236 |
| A571235Hi | 29696 | 1237 |
| A571236Hi | 29697 | 1238 |
| A571237Hi | 29722 | 1239 |
| A571238Hi | 29728 | 1240 |
| A571239Hi | 29729 | 1241 |
| A571240Hi | 29848 | 1242 |
| A571241Hi | 29849 | 1243 |
| A571242Hi | 29856 | 1244 |
| A571243Hi | 29862 | 1245 |
| A571244Hi | 29874 | 1246 |
| A571245Hi | 29875 | 1247 |
| A571246Hi | 29876 | 1248 |
| A571247Hi | 29877 | 1249 |
| A571248Hi | 29878 | 1250 |
| A571249Hi | 29920 | 1251 |
| 30001-30500 | | |
| A57070Hi | 30217 | 72 |
| A571250Hi | 30288 | 1252 |
| A571251Hi | 30289 | 1253 |
| A571252Hi | 30290 | 1254 |
| A571253Hi | 30291 | 1255 |
| A571254Hi | 30292 | 1256 |
| A571255Hi | 30293 | 1257 |
| A571256Hi | 30294 | 1258 |
| A571257Hi | 30294 | 1259 |
| A571258Hi | 30295 | 1260 |
| A571259Hi | 30295 | 1261 |
| A571260Hi | 30296 | 1262 |
| A571261Hi | 30296 | 1263 |
| A571262Hi | 30298 | 1264 |
| A571263Hi | 30299 | 1265 |
| A571264Hi | 30299 | 1266 |
| A571265Hi | 30300 | 1267 |
| A571266Hi | 30323 | 1268 |
| A571267Hi | 30324 | 1269 |
| A571268Hi | 30472 | 1270 |
| A571269Hi | 30498 | 1271 |
| A571270Hi | 30498 | 1272 |
| A57241Hi | 30286 | 243 |
| 30501-31000 | | |
| A571271Hi | 30536 | 1273 |
| A571272Hi | 30546 | 1274 |
| A571273Hi | 30770 | 1275 |
| A571274Hi | 30770 | 1276 |
| A571275Hi | 30772 | 1277 |
| A571276Hi | 30773 | 1278 |
| 31001-31500 | | |
| A57071Hi | 31035 | 73 |
| A571277Hi | 31023 | 1279 |
| A571278Hi | 31030 | 1280 |
| A571279Hi | 31034 | 1281 |
| A571280Hi | 31038 | 1282 |
| A571281Hi | 31046 | 1283 |
| A571282Hi | 31082 | 1284 |
| A571283Hi | 31103 | 1285 |
| A571284Hi | 31104 | 1286 |
| A571285Hi | 31131 | 1287 |
| A571286Hi | 31134 | 1288 |
| 32001-32500 | | |
| A57072Hi | 32398 | 74 |
| A571287H | 32292 | 1289 |
| A571288H | 32294 | 1290 |
| A571289H | 32296 | 1291 |
| A571290H | 32297 | 1292 |
| A571291H | 32301 | 1293 |
| A571292H | 32305 | 1294 |
| A571293H | 32307 | 1295 |
| A571294H | 32308 | 1296 |
| A571295H | 32312 | 1297 |
| A571296H | 32323 | 1298 |
| A571297H | 32329 | 1299 |
| A571298H | 32330 | 1300 |
| A571299H | 32331 | 1301 |
| A571300H | 32347 | 1302 |
| A571301H | 32351 | 1303 |
| A571302H | 32360 | 1304 |
| A571303H | 32363 | 1305 |
| A571304Hi | 32392 | 1306 |
| A571305Hi | 32405 | 1307 |
| A571306Hi | 32423 | 1308 |
| A571307Hi | 32424 | 1309 |
| A571308Hi | 32424 | 1310 |
| A571309Hi | 32443 | 1311 |
| 32501-33000 | | |
| A57073Hi | 32529 | 75 |
| A57074Hi | 32569 | 76 |
| A57075Hi | 32611 | 77 |
| A571310Hi | 32510 | 1312 |
| A571311Hi | 32511 | 1313 |
| A571312Hi | 32518 | 1314 |
| A571313Hi | 32518 | 1315 |
| A571314Hi | 32519 | 1316 |
| A571315Hi | 32520 | 1317 |
| A571316Hi | 32521 | 1318 |
| A571317Hi | 32522 | 1319 |
| A571318Hi | 32528 | 1320 |
| A571319Hi | 32545 | 1321 |
| A571320Hi | 32560 | 1322 |
| A571321Hi | 32565 | 1323 |
| A571322Hi | 32565 | 1324 |
| A571323Hi | 32566 | 1325 |
| A571324Hi | 32570 | 1326 |
| A571325Hi | 32571 | 1327 |
| A571326Hi | 32577 | 1328 |
| A571327Hi | 32580 | 1329 |
| A571328Hi | 32608 | 1330 |
| A571329Hi | 32641 | 1331 |
| A571330Hi | 32738 | 1332 |
| A571331Hi | 32738 | 1333 |
| A571332Hi | 32743 | 1334 |
| A571333Hi | 32823 | 1335 |
| A571334Hi | 32823 | 1336 |
| A571335Hi | 32824 | 1337 |
| A571336Hi | 32826 | 1338 |
| A571337Hi | 32874 | 1339 |
| A571338Hi | 32875 | 1340 |
| A57242Hi | 32561 | 244 |
| A57243Hi | 32564 | 245 |
| A57244Hi | 32579 | 246 |
| 33001-33500 | | |
| A57076Hi | 33116 | 78 |
| A57077Hi | 33120 | 79 |
| A571339Hi | 33039 | 1341 |
| A571340Hi | 33039 | 1342 |
| A571341Hi | 33040 | 1343 |
| A571342Hi | 33047 | 1344 |
| A571343Hi | 33050 | 1345 |
| A571344Hi | 33051 | 1346 |
| A571345Hi | 33051 | 1347 |
| A571346Hi | 33108 | 1348 |
| A571347Hi | 33121 | 1349 |
| A571348Hi | 33130 | 1350 |
| A571349Hi | 33168 | 1351 |
| A571350Hi | 33281 | 1352 |
| A571351Hi | 33283 | 1353 |
| A571352Hi | 33476 | 1354 |
| A571353Hi | 33478 | 1355 |
| A571354Hi | 33478 | 1356 |
| A571355Hi | 33480 | 1357 |
| A571356Hi | 33481 | 1358 |
| A571357Hi | 33481 | 1359 |
| A571358Hi | 33487 | 1360 |
| A571359Hi | 33492 | 1361 |
| A571360Hi | 33493 | 1362 |
| A571361Hi | 33494 | 1363 |
| 33501-34000 | | |
| A571362Hi | 33653 | 1364 |
| A571363Hi | 33803 | 1365 |
| A571364Hi | 33806 | 1366 |
| A571365Hi | 33807 | 1367 |
| A571366Hi | 33813 | 1368 |
| A571367Hi | 33814 | 1369 |
| A571368Hi | 33815 | 1370 |
| A571369Hi | 33837 | 1371 |
| A571370Hi | 33885 | 1372 |
| A571371Hi | 33886 | 1373 |
| A571372Hi | 33887 | 1374 |
| A571373Hi | 33890 | 1375 |
| A571374Hi | 33891 | 1376 |
| A571375Hi | 33895 | 1377 |
| A571376Hi | 33895 | 1378 |
| A571377Hi | 33920 | 1379 |
| A571378Hi | 33921 | 1380 |
| A571379Hi | 33923 | 1381 |
| A571380Hi | 33953 | 1382 |
| 34001-34500 | | |
| A57078Hi | 34021 | 80 |
| A57079Hi | 34026 | 81 |
| A57080Hi | 34038 | 82 |
| A571381Hi | 34024 | 1383 |
| A571382Hi | 34029 | 1384 |
| A571383Hi | 34110 | 1385 |
| A571384Hi | 34141 | 1386 |
| A571385Hi | 34142 | 1387 |
| A571386Hi | 34297 | 1388 |
| A571387Hi | 34417 | 1389 |
| A571388Hi | 34418 | 1390 |
| A571389Hi | 34427 | 1391 |
| 34501-35000 | | |
| A57081Hi | 34942 | 83 |
| A571390H | 34543 | 1392 |
| A571391H | 34543 | 1393 |
| A571392H | 34545 | 1394 |
| A571393H | 34546 | 1395 |
| A571394H | 34546 | 1396 |
| A571395H | 34548 | 1397 |
| A571396H | 34550 | 1398 |
| A571397H | 34551 | 1399 |
| A571398H | 34555 | 1400 |
| A571399H | 34557 | 1401 |
| A571400H | 34558 | 1402 |
| A571401H | 34559 | 1403 |
| A571402H | 34560 | 1404 |
| A571403H | 34563 | 1405 |
| A571404H | 34564 | 1406 |
| A571405H | 34565 | 1407 |
| A571406H | 34566 | 1408 |
| A571407H | 34566 | 1409 |
| A571408H | 34567 | 1410 |
| A571409H | 34567 | 1411 |
| A571410H | 34568 | 1412 |
| A571411H | 34568 | 1413 |
| A571412H | 34568 | 1414 |
| A571413H | 34569 | 1415 |
| A571414H | 34569 | 1416 |
| A571415H | 34569 | 1417 |
| A571416H | 34570 | 1418 |
| A571417H | 34570 | 1419 |
| A571418H | 34570 | 1420 |
| A571419H | 34571 | 1421 |
| A571420H | 34571 | 1422 |
| A571421H | 34571 | 1423 |
| A571422H | 34571 | 1424 |
| A571423H | 34572 | 1425 |
| A571424H | 34572 | 1426 |
| A571425H | 34573 | 1427 |
| A571426H | 34573 | 1428 |
| A571427H | 34573 | 1429 |
| A571428H | 34574 | 1430 |
| A571429H | 34574 | 1431 |
| A571430H | 34574 | 1432 |
| A571431H | 34574 | 1433 |
| A571432H | 34575 | 1434 |
| A571433H | 34575 | 1435 |
| A571434H | 34575 | 1436 |
| A571435H | 34576 | 1437 |
| A571436H | 34576 | 1438 |
| A571437H | 34576 | 1439 |
| A571438H | 34577 | 1440 |
| A571439Hi | 34610 | 1441 |
| A571440Hi | 34611 | 1442 |
| A571441Hi | 34943 | 1443 |
| A571442Hi | 34943 | 1444 |
| A57255H | 34567 | 257 |
| 35001-35500 | | |
| A571443Hi | 35351 | 1445 |
| A571444Hi | 35392 | 1446 |
| 35501-36000 | | |
| A571445Hi | 35637 | 1447 |
| A571446Hi | 35800 | 1448 |
| A571447Hi | 35928 | 1449 |
| A571448Hi | 35930 | 1450 |
| 36001-36500 | | |
| A57082Hi | 36460 | 84 |
| A571449Hi | 36017 | 1451 |
| A571450Hi | 36019 | 1452 |
| A571451Hi | 36103 | 1453 |
| A571452Hi | 36238 | 1454 |
| A571453Hi | 36239 | 1455 |
| A571454Hi | 36242 | 1456 |
| A571455Hi | 36462 | 1457 |
| A571456Hi | 36463 | 1458 |
| 36501-37000 | | |
| A57083Hi | 36843 | 85 |
| A57084Hi | 36848 | 86 |
| A571457Hi | 36508 | 1459 |
| A571458Hi | 36510 | 1460 |
| A571459Hi | 36511 | 1461 |
| A571460Hi | 36512 | 1462 |
| A571461Hi | 36513 | 1463 |
| A571462Hi | 36539 | 1464 |
| A571463Hi | 36541 | 1465 |
| A571464Hi | 36542 | 1466 |
| A571465Hi | 36616 | 1467 |
| A571466Hi | 36620 | 1468 |
| A571467Hi | 36746 | 1469 |
| A571468Hi | 36843 | 1470 |
| A571469Hi | 36846 | 1471 |
| A571470Hi | 36850 | 1472 |
| 37001-37500 | | |
| A57085Hi | 37013 | 87 |
| A571471Hi | 37398 | 1473 |
| A571472Hi | 37401 | 1474 |
| A571473Hi | 37402 | 1475 |
| A571474Hi | 37405 | 1476 |
| A571475Hi | 37406 | 1477 |
| A571476Hi | 37408 | 1478 |
| A571477Hi | 37408 | 1479 |
| A571478Hi | 37410 | 1480 |
| A571479Hi | 37412 | 1481 |
| A571480Hi | 37444 | 1482 |
| A571481Hi | 37495 | 1483 |
| A571482Hi | 37496 | 1484 |
| A571483Hi | 37497 | 1485 |
| A571484Hi | 37498 | 1486 |
| 37501-38000 | | |
| A57086Hi | 37688 | 88 |
| A57087Hi | 37896 | 89 |
| A571485Hi | 37501 | 1487 |
| A571486Hi | 37510 | 1488 |
| A571487Hi | 37510 | 1489 |
| A571488Hi | 37522 | 1490 |
| A571489Hi | 37647 | 1491 |
| A571490Hi | 37648 | 1492 |
| A571491Hi | 37653 | 1493 |
| A571492Hi | 37686 | 1494 |
| A571493Hi | 37687 | 1495 |
| A571494Hi | 37691 | 1496 |
| A571495Hi | 37738 | 1497 |
| A571496Hi | 37739 | 1498 |
| A571497Hi | 37803 | 1499 |
| A571498Hi | 37804 | 1500 |
| A571499Hi | 37893 | 1501 |
| A571500Hi | 37896 | 1502 |
| A571501Hi | 37925 | 1503 |
| A571502Hi | 37928 | 1504 |
| A571503Hi | 37953 | 1505 |
| A571504Hi | 37954 | 1506 |
| 38001-38500 | | |
| A57088Hi | 38085 | 90 |
| A571505Hi | 38002 | 1507 |
| A571506Hi | 38365 | 1508 |
| A571507Hi | 38365 | 1509 |
| A571508Hi | 38472 | 1510 |
| A571509Hi | 38474 | 1511 |
| A571510Hi | 38481 | 1512 |
| A571511Hi | 38482 | 1513 |
| A571512Hi | 38484 | 1514 |
| A571513Hi | 38485 | 1515 |
| A571514Hi | 38486 | 1516 |
| 38501-39000 | | |
| A571515Hi | 38564 | 1517 |
| A571516Hi | 38566 | 1518 |
| A571517Hi | 38728 | 1519 |
| A571518Hi | 38729 | 1520 |
| A571519Hi | 38734 | 1521 |
| A571520Hi | 38735 | 1522 |
| A571521H | 38793 | 1523 |
| A571522H | 38841 | 1524 |
| A571523H | 38852 | 1525 |
| A571524H | 38855 | 1526 |
| A571525H | 38857 | 1527 |
| A571526H | 38859 | 1528 |
| A571527H | 38863 | 1529 |
| A571528H | 38864 | 1530 |
| A571529H | 38865 | 1531 |
| A571530H | 38866 | 1532 |
| A571531H | 38871 | 1533 |
| A571532H | 38872 | 1534 |
| A571533H | 38874 | 1535 |
| A571534H | 38875 | 1536 |
| A571535H | 38875 | 1537 |
| A571536H | 38876 | 1538 |
| A571537H | 38876 | 1539 |
| A571538H | 38876 | 1540 |
| A571539H | 38877 | 1541 |
| A571540H | 38877 | 1542 |
| A571541H | 38878 | 1543 |
| A571542H | 38879 | 1544 |
| A571543H | 38882 | 1545 |
| A571544H | 38883 | 1546 |
| A571545H | 38883 | 1547 |
| A571546H | 38884 | 1548 |
| A571547H | 38885 | 1549 |
| A571548H | 38885 | 1550 |
| A571549H | 38887 | 1551 |
| A571550H | 38888 | 1552 |
| 39001-39500 | | |
| A57089Hi | 39087 | 91 |
| A571551Hi | 39080 | 1553 |
| A571552Hi | 39081 | 1554 |
| A571553Hi | 39152 | 1555 |
| 39501-40000 | | |
| A571554Hi | 39709 | 1556 |
| A571555Hi | 39907 | 1557 |
| A571556Hi | 39983 | 1558 |
| 40001-40500 | | |
| A57090Hi | 40151 | 92 |
| A571557Hi | 40024 | 1559 |
| A571558Hi | 40096 | 1560 |
| A571559Hi | 40099 | 1561 |
| A571560Hi | 40102 | 1562 |
| A571561Hi | 40119 | 1563 |
| A571562Hi | 40150 | 1564 |
| A571563Hi | 40153 | 1565 |
| A571564Hi | 40153 | 1566 |
| A571565Hi | 40158 | 1567 |
| A571566Hi | 40159 | 1568 |
| A571567Hi | 40356 | 1569 |
| A571568Hi | 40357 | 1570 |
| A571569Hi | 40358 | 1571 |
| 40501-41000 | | |
| A571570Hi | 40556 | 1572 |
| 42001-42500 | | |
| A57091Hi | 42242 | 93 |
| A571571Hi | 42238 | 1573 |
| A571572Hi | 42240 | 1574 |
| A571573Hi | 42241 | 1575 |
| A571574Hi | 42242 | 1576 |
| A571575Hi | 42247 | 1577 |
| A571576Hi | 42247 | 1578 |
| A571577Hi | 42248 | 1579 |
| A571578Hi | 42249 | 1580 |
| A571579Hi | 42249 | 1581 |
| A571580Hi | 42250 | 1582 |
| A571581Hi | 42250 | 1583 |
| A571582Hi | 42369 | 1584 |
| A571583Hi | 42372 | 1585 |
| A571584Hi | 42429 | 1586 |
| 42501-43000 | | |
| A57092Hi | 42832 | 94 |
| A57093Hi | 42880 | 95 |
| A57094Hi | 42922 | 96 |
| A57095Hi | 42952 | 97 |
| A57096Hi | 42954 | 98 |
| A57097Hi | 42958 | 99 |
| A571585Hi | 42505 | 1587 |
| A571586Hi | 42535 | 1588 |
| A571587Hi | 42535 | 1589 |
| A571588Hi | 42536 | 1590 |
| A571589Hi | 42570 | 1591 |
| A571590Hi | 42571 | 1592 |
| A571591Hi | 42650 | 1593 |
| A571592Hi | 42650 | 1594 |
| A571593Hi | 42650 | 1595 |
| A571594Hi | 42833 | 1596 |
| A571595Hi | 42833 | 1597 |
| A571596Hi | 42838 | 1598 |
| A571597Hi | 42880 | 1599 |
| A571598Hi | 42881 | 1600 |
| A571599Hi | 42882 | 1601 |
| A571600Hi | 42925 | 1602 |
| A571601Hi | 42941 | 1603 |
| A571602Hi | 42955 | 1604 |
| A571603Hi | 42956 | 1605 |
| A571604Hi | 42957 | 1606 |
| A571605Hi | 42958 | 1607 |
| A571606Hi | 42968 | 1608 |
| 43001-43500 | | |
| A57098Hi | 43090 | 100 |
| A57099Hi | 43259 | 101 |
| A57100Hi | 43263 | 102 |
| A571607Hi | 43022 | 1609 |
| A571608Hi | 43023 | 1610 |
| A571609Hi | 43076 | 1611 |
| A571610Hi | 43077 | 1612 |
| A571611Hi | 43079 | 1613 |
| A571612Hi | 43083 | 1614 |
| A571613Hi | 43084 | 1615 |
| A571614Hi | 43084 | 1616 |
| A571615Hi | 43085 | 1617 |
| A571616Hi | 43088 | 1618 |
| A571617Hi | 43089 | 1619 |
| A571618Hi | 43089 | 1620 |
| A571619Hi | 43129 | 1621 |
| A571620Hi | 43131 | 1622 |
| A571621Hi | 43135 | 1623 |
| A571622Hi | 43141 | 1624 |
| A571623Hi | 43142 | 1625 |
| A571624Hi | 43170 | 1626 |
| A571625Hi | 43265 | 1627 |
| A571626Hi | 43267 | 1628 |
| 43501-44000 | | |
| A57101Hi | 43904 | 103 |
| A571627Hi | 43584 | 1629 |
| A571628Hi | 43627 | 1630 |
| A571629Hi | 43634 | 1631 |
| A571630Hi | 43714 | 1632 |
| A571631Hi | 43714 | 1633 |
| A571632Hi | 43717 | 1634 |
| A571633Hi | 43772 | 1635 |
| A571634Hi | 43773 | 1636 |
| A571635Hi | 43849 | 1637 |
| A571636Hi | 43852 | 1638 |
| A571637Hi | 43857 | 1639 |
| A571638Hi | 43858 | 1640 |
| A571639Hi | 43859 | 1641 |
| A571640Hi | 43859 | 1642 |
| A571641Hi | 43896 | 1643 |
| A571642Hi | 43898 | 1644 |
| A571643Hi | 43902 | 1645 |
| A571644Hi | 43904 | 1646 |
| A571645Hi | 43905 | 1647 |
| A571646Hi | 43906 | 1648 |
| A571647Hi | 43909 | 1649 |
| A571648Hi | 43910 | 1650 |
| A571649Hi | 43911 | 1651 |
| A571650Hi | 43984 | 1652 |
| A571651Hi | 43986 | 1653 |
| 44001-44500 | | |
| A57102Hi | 44172 | 104 |
| A57103Hi | 44337 | 105 |
| A571652Hi | 44053 | 1654 |
| A571653Hi | 44054 | 1655 |
| A571654Hi | 44055 | 1656 |
| A571655Hi | 44057 | 1657 |
| A571656Hi | 44138 | 1658 |
| A571657Hi | 44139 | 1659 |
| A571658Hi | 44268 | 1660 |
| A571659Hi | 44270 | 1661 |
| A571660Hi | 44340 | 1662 |
| A571661Hi | 44492 | 1663 |
| A571662Hi | 44493 | 1664 |
| A571663Hi | 44494 | 1665 |
| A571664Hi | 44494 | 1666 |
| A571665Hi | 44495 | 1667 |
| A571666Hi | 44495 | 1668 |
| 44501-45000 | | |
| A57104Hi | 44934 | 106 |
| A571667Hi | 44513 | 1669 |
| A571668Hi | 44516 | 1670 |
| A571669Hi | 44522 | 1671 |
| A571670Hi | 44523 | 1672 |
| A571671Hi | 44523 | 1673 |
| A571672Hi | 44524 | 1674 |
| A571673Hi | 44525 | 1675 |
| A571674Hi | 44525 | 1676 |
| A571675Hi | 44535 | 1677 |
| A571676Hi | 44593 | 1678 |
| A571677Hi | 44643 | 1679 |
| A571678Hi | 44650 | 1680 |
| A571679Hi | 44655 | 1681 |
| A571680Hi | 44657 | 1682 |
| A571681Hi | 44704 | 1683 |
| A571682Hi | 44708 | 1684 |
| A571683Hi | 44713 | 1685 |
| A571684Hi | 44714 | 1686 |
| A571685Hi | 44930 | 1687 |
| A571686Hi | 44934 | 1688 |
| 45001-45500 | | |
| A571687Hi | 45010 | 1689 |
| A571688Hi | 45011 | 1690 |
| A571689Hi | 45011 | 1691 |
| A571690Hi | 45368 | 1692 |
| A571691Hi | 45370 | 1693 |
| A571692Hi | 45371 | 1694 |
| A571693Hi | 45377 | 1695 |
| A571694Hi | 45378 | 1696 |
| 45501-46000 | | |
| A57105Hi | 45910 | 107 |
| A571695Hi | 45576 | 1697 |
| A571696Hi | 45577 | 1698 |
| A571697Hi | 45732 | 1699 |
| A571698Hi | 45817 | 1700 |
| A571699Hi | 45916 | 1701 |
| A571700Hi | 45921 | 1702 |
| 46001-46500 | | |
| A571701Hi | 46128 | 1703 |
| A571702Hi | 46129 | 1704 |
| A571703Hi | 46130 | 1705 |
| A571704Hi | 46138 | 1706 |
| A571705Hi | 46358 | 1707 |
| A571706Hi | 46362 | 1708 |
| A571707Hi | 46362 | 1709 |
| A571708Hi | 46363 | 1710 |
| A571709Hi | 46364 | 1711 |
| A571710Hi | 46365 | 1712 |
| A571711Hi | 46366 | 1713 |
| A571712Hi | 46496 | 1714 |
| 46501-47000 | | |
| A571713Hi | 46501 | 1715 |
| A571714Hi | 46502 | 1716 |
| A571715Hi | 46506 | 1717 |
| A571716Hi | 46507 | 1718 |
| A571717Hi | 46507 | 1719 |
| A571718Hi | 46514 | 1720 |
| A571719Hi | 46515 | 1721 |
| A571720Hi | 46515 | 1722 |
| A571721Hi | 46518 | 1723 |
| A571722Hi | 46519 | 1724 |
| A571723Hi | 46525 | 1725 |
| A571724Hi | 46528 | 1726 |
| A571725Hi | 46549 | 1727 |
| A571726Hi | 46552 | 1728 |
| A571727Hi | 46888 | 1729 |
| A571728Hi | 46988 | 1730 |
| A571729Hi | 46989 | 1731 |
| 47001-47500 | | |
| A57106Hi | 47251 | 108 |
| A571730Hi | 47247 | 1732 |
| A571731Hi | 47256 | 1733 |
| A571732Hi | 47371 | 1734 |
| A571733Hi | 47487 | 1735 |
| A571734Hi | 47488 | 1736 |
| 47501-48000 | | |
| A571735Hi | 47561 | 1737 |
| A571736Hi | 47757 | 1738 |
| A571737Hi | 47973 | 1739 |
| A571738Hi | 47976 | 1740 |
| 48001-48500 | | |
| A57107Hi | 48387 | 109 |
| A571739Hi | 48124 | 1741 |
| A571740Hi | 48233 | 1742 |
| A571741Hi | 48249 | 1743 |
| A571742Hi | 48252 | 1744 |
| A571743Hi | 48291 | 1745 |
| A571744Hi | 48292 | 1746 |
| A571745Hi | 48292 | 1747 |
| A571746Hi | 48293 | 1748 |
| A571747Hi | 48294 | 1749 |
| A571748Hi | 48296 | 1750 |
| A571749Hi | 48297 | 1751 |
| A571750Hi | 48297 | 1752 |
| A571751Hi | 48299 | 1753 |
| A571752Hi | 48320 | 1754 |
| A571753Hi | 48383 | 1755 |
| A571754Hi | 48384 | 1756 |
| A571755Hi | 48384 | 1757 |
| A571756Hi | 48386 | 1758 |
| A571757Hi | 48387 | 1759 |
| A571758Hi | 48388 | 1760 |
| A571759Hi | 48397 | 1761 |
| A571760Hi | 48479 | 1762 |
| 48501-49000 | | |
| A57108Hi | 48522 | 110 |
| A57109Hi | 48530 | 111 |
| A57110Hi | 48605 | 112 |
| A57111Hi | 48638 | 113 |
| A57112Hi | 48812 | 114 |
| A57113Hi | 48813 | 115 |
| A57114Hi | 48851 | 116 |
| A57115Hi | 48852 | 117 |
| A57116Hi | 48933 | 118 |
| A57117Hi | 48934 | 119 |
| A571761Hi | 48527 | 1763 |
| A571762Hi | 48527 | 1764 |
| A571763Hi | 48529 | 1765 |
| A571764Hi | 48536 | 1766 |
| A571765Hi | 48556 | 1767 |
| A571766Hi | 48606 | 1768 |
| A571767Hi | 48619 | 1769 |
| A571768Hi | 48620 | 1770 |
| A571769Hi | 48621 | 1771 |
| A571770Hi | 48621 | 1772 |
| A571771Hi | 48622 | 1773 |
| A571772Hi | 48623 | 1774 |
| A571773Hi | 48624 | 1775 |
| A571774Hi | 48624 | 1776 |
| A571775Hi | 48625 | 1777 |
| A571776Hi | 48628 | 1778 |
| A571777Hi | 48629 | 1779 |
| A571778Hi | 48630 | 1780 |
| A571779Hi | 48630 | 1781 |
| A571780Hi | 48635 | 1782 |
| A571781Hi | 48636 | 1783 |
| A571782Hi | 48636 | 1784 |
| A571783Hi | 48637 | 1785 |
| A571784Hi | 48638 | 1786 |
| A571785Hi | 48639 | 1787 |
| A571786Hi | 48640 | 1788 |
| A571787Hi | 48640 | 1789 |
| A571788Hi | 48641 | 1790 |
| A571789Hi | 48642 | 1791 |
| A571790Hi | 48646 | 1792 |
| A571791Hi | 48807 | 1793 |
| A571792Hi | 48848 | 1794 |
| A57245Hi | 48546 | 247 |
| 49001-49500 | | |
| A571793Hi | 49060 | 1795 |
| A571794Hi | 49064 | 1796 |
| A571795Hi | 49065 | 1797 |
| A571796Hi | 49066 | 1798 |
| A571797Hi | 49068 | 1799 |
| A571798Hi | 49069 | 1800 |
| A571799Hi | 49316 | 1801 |
| A571800Hi | 49412 | 1802 |
| 49501-50000 | | |
| A57118Hi | 49537 | 120 |
| A57119Hi | 49998 | 121 |
| A571801Hi | 49515 | 1803 |
| A571802Hi | 49518 | 1804 |
| A571803Hi | 49519 | 1805 |
| A571804Hi | 49520 | 1806 |
| A571805Hi | 49524 | 1807 |
| A571806Hi | 49525 | 1808 |
| A571807Hi | 49525 | 1809 |
| A571808Hi | 49526 | 1810 |
| A571809Hi | 49528 | 1811 |
| A571810H | 49596 | 1812 |
| A571811H | 49604 | 1813 |
| A571812H | 49609 | 1814 |
| A571813H | 49699 | 1815 |
| A571814H | 49700 | 1816 |
| A571815H | 49702 | 1817 |
| A571816H | 49705 | 1818 |
| A571817H | 49706 | 1819 |
| A571818H | 49708 | 1820 |
| A571819H | 49712 | 1821 |
| A571820H | 49713 | 1822 |
| A571821H | 49861 | 1823 |
| A571822H | 49863 | 1824 |
| A571823H | 49869 | 1825 |
| A571824H | 49870 | 1826 |
| A571825H | 49871 | 1827 |
| A571826H | 49871 | 1828 |
| A571827H | 49872 | 1829 |
| A571828H | 49872 | 1830 |
| A571829H | 49916 | 1831 |
| A571830H | 49916 | 1832 |
| A571831H | 49916 | 1833 |
| A571832H | 49917 | 1834 |
| A571833H | 49917 | 1835 |
| A571834H | 49918 | 1836 |
| A571835H | 49918 | 1837 |
| A571836H | 49919 | 1838 |
| A571837H | 49920 | 1839 |
| A571838H | 49921 | 1840 |
| A571839H | 49921 | 1841 |
| A571840H | 49922 | 1842 |
| A571841H | 49922 | 1843 |
| A571842H | 49927 | 1844 |
| A571843H | 49930 | 1845 |
| A571844H | 49952 | 1846 |
| A571845H | 49953 | 1847 |
| A571846H | 49954 | 1848 |
| A571847H | 49954 | 1849 |
| A571848H | 49955 | 1850 |
| A571849H | 49955 | 1851 |
| A571850H | 49956 | 1852 |
| A571851H | 49956 | 1853 |
| A571852H | 49957 | 1854 |
| A571853H | 49957 | 1855 |
| A571854H | 49957 | 1856 |
| A571855H | 49958 | 1857 |
| A571856H | 49958 | 1858 |
| A571857H | 49959 | 1859 |
| A571858H | 49960 | 1860 |
| A571859H | 49961 | 1861 |
| A571860H | 49961 | 1862 |
| A571861H | 49962 | 1863 |
| A571862H | 49962 | 1864 |
| A571863H | 49963 | 1865 |
| A571864H | 49964 | 1866 |
| A571865H | 49964 | 1867 |
| A571866H | 49965 | 1868 |
| A571867H | 49967 | 1869 |
| A571868Hi | 49998 | 1870 |
| 50001-50500 | | |
| A571869Hi | 50151 | 1871 |
| A571870Hi | 50160 | 1872 |
| A571871Hi | 50188 | 1873 |
| A571872Hi | 50189 | 1874 |
| 50501-51000 | | |
| A571873Hi | 50587 | 1875 |
| A571874Hi | 50590 | 1876 |
| A571875Hi | 50937 | 1877 |
| A571876Hi | 50938 | 1878 |
| A571877Hi | 50940 | 1879 |
| A571878Hi | 50941 | 1880 |
| A571879Hi | 50942 | 1881 |
| A571880Hi | 50942 | 1882 |
| A571881Hi | 50971 | 1883 |
| 51001-51500 | | |
| A57120Hi | 51201 | 122 |
| A571882Hi | 51097 | 1884 |
| 51501-52000 | | |
| A571883Hi | 51727 | 1885 |
| A571884Hi | 51728 | 1886 |
| A571885Hi | 51745 | 1887 |
| A571886Hi | 51746 | 1888 |
| A571887Hi | 51747 | 1889 |
| A571888Hi | 51748 | 1890 |
| A571889Hi | 51757 | 1891 |
| 52001-52500 | | |
| A57121Hi | 52279 | 123 |
| A571890Hi | 52275 | 1892 |
| A571891Hi | 52275 | 1893 |
| A571892Hi | 52461 | 1894 |
| A571893Hi | 52466 | 1895 |
| 52501-53000 | | |
| A571894Hi | 52554 | 1896 |
| A571895Hi | 52555 | 1897 |
| A571896Hi | 52590 | 1898 |
| A571897Hi | 52599 | 1899 |
| A571898Hi | 52705 | 1900 |
| A571899Hi | 52772 | 1901 |
| A571900Hi | 52777 | 1902 |
| A571901Hi | 52778 | 1903 |
| A571902Hi | 52780 | 1904 |
| A571903Hi | 52783 | 1905 |
| A571904Hi | 52786 | 1906 |
| A571905Hi | 52790 | 1907 |
| A571906Hi | 52791 | 1908 |
| A571907Hi | 52794 | 1909 |
| A571908Hi | 52796 | 1910 |
| A571909Hi | 52798 | 1911 |
| A571910Hi | 52801 | 1912 |
| A571911Hi | 52819 | 1913 |
| A571912H | 52892 | 1914 |
| A571913H | 52893 | 1915 |
| A571914H | 52895 | 1916 |
| A571915H | 52896 | 1917 |
| A571916H | 52897 | 1918 |
| A571917H | 52899 | 1919 |
| A571918H | 52900 | 1920 |
| A571919H | 52900 | 1921 |
| A571920H | 52909 | 1922 |
| A571921H | 52910 | 1923 |
| A571922H | 52910 | 1924 |
| A571923H | 52911 | 1925 |
| A571924H | 52912 | 1926 |
| A571925H | 52912 | 1927 |
| A571926H | 52913 | 1928 |
| A571927H | 52914 | 1929 |
| A571928H | 52915 | 1930 |
| A571929H | 52915 | 1931 |
| A571930H | 52916 | 1932 |
| A571931H | 52916 | 1933 |
| A571932H | 52922 | 1934 |
| A571933H | 52923 | 1935 |
| A571934H | 52924 | 1936 |
| A571935H | 52924 | 1937 |
| A571936H | 52925 | 1938 |
| A571937H | 52932 | 1939 |
| A571938H | 52934 | 1940 |
| A571939H | 52935 | 1941 |
| A571940H | 52937 | 1942 |
| A571941H | 52997 | 1943 |
| 53001-53500 | | |
| A571942H | 53002 | 1944 |
| A571943Hi | 53037 | 1945 |
| A571944Hi | 53052 | 1946 |
| A571945Hi | 53053 | 1947 |
| A571946H | 53096 | 1948 |
| A571947H | 53098 | 1949 |
| A571948H | 53102 | 1950 |
| A571949H | 53108 | 1951 |
| A571950H | 53109 | 1952 |
| A571951H | 53110 | 1953 |
| A571952H | 53117 | 1954 |
| A571953H | 53119 | 1955 |
| A571954H | 53120 | 1956 |
| A571955H | 53157 | 1957 |
| A571956H | 53158 | 1958 |
| A571957H | 53159 | 1959 |
| A571958H | 53159 | 1960 |
| A571959H | 53160 | 1961 |
| A571960H | 53160 | 1962 |
| A571961H | 53161 | 1963 |
| A571962H | 53162 | 1964 |
| A571963H | 53162 | 1965 |
| A571964H | 53163 | 1966 |
| A571965H | 53164 | 1967 |
| A571966H | 53165 | 1968 |
| A571967H | 53165 | 1969 |
| A571968H | 53166 | 1970 |
| A571969H | 53167 | 1971 |
| A571970H | 53168 | 1972 |
| A571971H | 53168 | 1973 |
| A571972H | 53169 | 1974 |
| A571973H | 53169 | 1975 |
| A571974H | 53171 | 1976 |
| A571975H | 53178 | 1977 |
| A571976H | 53182 | 1978 |
| A571977H | 53183 | 1979 |
| A571978H | 53184 | 1980 |
| A571979H | 53185 | 1981 |
| A571980H | 53187 | 1982 |
| A571981H | 53188 | 1983 |
| A571982H | 53191 | 1984 |
| A571983H | 53192 | 1985 |
| A571984H | 53193 | 1986 |
| A571985H | 53218 | 1987 |
| A571986H | 53219 | 1988 |
| A571987H | 53220 | 1989 |
| A571988H | 53220 | 1990 |
| A571989H | 53223 | 1991 |
| A571990H | 53224 | 1992 |
| A571991H | 53224 | 1993 |
| A571992H | 53225 | 1994 |
| A571993H | 53226 | 1995 |
| A571994H | 53226 | 1996 |
| A571995H | 53229 | 1997 |
| A571996Hi | 53350 | 1998 |
| A571997Hi | 53351 | 1999 |
| A571998Hi | 53419 | 2000 |
| A571999Hi | 53458 | 2001 |
| A57256H | 53001 | 258 |
| 53501-54000 | | |
| A57122Hi | 53941 | 124 |
| A572000Hi | 53524 | 2002 |
| A572001Hi | 53530 | 2003 |
| A572002Hi | 53550 | 2004 |
| A572003Hi | 53551 | 2005 |
| A572004Hi | 53562 | 2006 |
| A572005Hi | 53564 | 2007 |
| 54001-54500 | | |
| A572006Hi | 54093 | 2008 |
| A572007Hi | 54100 | 2009 |
| A572008Hi | 54103 | 2010 |
| A572009Hi | 54109 | 2011 |
| A572010Hi | 54300 | 2012 |
| A572011Hi | 54301 | 2013 |
| A572012Hi | 54395 | 2014 |
| A572013Hi | 54493 | 2015 |
| 54501-55000 | | |
| A57123Hi | 54906 | 125 |
| A572014Hi | 54531 | 2016 |
| A572015Hi | 54533 | 2017 |
| A572016Hi | 54594 | 2018 |
| A572017Hi | 54596 | 2019 |
| A572018Hi | 54599 | 2020 |
| A572019Hi | 54601 | 2021 |
| A572020Hi | 54602 | 2022 |
| A572021Hi | 54606 | 2023 |
| A572022Hi | 54799 | 2024 |
| A572023Hi | 54871 | 2025 |
| A572024Hi | 54875 | 2026 |
| 55001-55500 | | |
| A57124Hi | 55121 | 126 |
| A57125Hi | 55140 | 127 |
| A572025Hi | 55118 | 2027 |
| A572026Hi | 55120 | 2028 |
| A572027Hi | 55316 | 2029 |
| 56001-56500 | | |
| A57126Hi | 56373 | 128 |
| A57127Hi | 56375 | 129 |
| A572028Hi | 56182 | 2030 |
| A572029Hi | 56182 | 2031 |
| A572030Hi | 56183 | 2032 |
| A572031Hi | 56183 | 2033 |
| A572032Hi | 56227 | 2034 |
| A572033Hi | 56294 | 2035 |
| A572034Hi | 56296 | 2036 |
| A572035Hi | 56299 | 2037 |
| A572036Hi | 56300 | 2038 |
| A572037Hi | 56355 | 2039 |
| A572038Hi | 56355 | 2040 |
| A572039Hi | 56357 | 2041 |
| A572040Hi | 56358 | 2042 |
| A572041Hi | 56361 | 2043 |
| A572042Hi | 56362 | 2044 |
| A572043Hi | 56364 | 2045 |
| A572044Hi | 56367 | 2046 |
| A572045Hi | 56367 | 2047 |
| A572046Hi | 56370 | 2048 |
| A572047Hi | 56373 | 2049 |
| A572048Hi | 56469 | 2050 |
| 56501-57000 | | |
| A57128Hi | 56645 | 130 |
| A57129Hi | 56742 | 131 |
| A57130Hi | 56749 | 132 |
| A572049Hi | 56623 | 2051 |
| A572050Hi | 56637 | 2052 |
| A572051Hi | 56638 | 2053 |
| A572052Hi | 56643 | 2054 |
| A572053Hi | 56645 | 2055 |
| A572054Hi | 56740 | 2056 |
| A572055Hi | 56740 | 2057 |
| A572056Hi | 56748 | 2058 |
| A572057Hi | 56750 | 2059 |
| A572058Hi | 56751 | 2060 |
| A572059Hi | 56758 | 2061 |
| A572060Hi | 56765 | 2062 |
| A572061Hi | 56765 | 2063 |
| A572062Hi | 56766 | 2064 |
| A572063Hi | 56790 | 2065 |
| A572064Hi | 56813 | 2066 |
| A572065Hi | 56907 | 2067 |
| A572066Hi | 56908 | 2068 |
| A572067Hi | 56908 | 2069 |
| A572068Hi | 56911 | 2070 |
| A572069Hi | 56912 | 2071 |
| A572070Hi | 56921 | 2072 |
| A572071Hi | 56921 | 2073 |
| 57001-57500 | | |
| A572072Hi | 57305 | 2074 |
| A572073Hi | 57306 | 2075 |
| A572074Hi | 57306 | 2076 |
| A572075Hi | 57314 | 2077 |
| A572076Hi | 57354 | 2078 |
| A572077Hi | 57390 | 2079 |
| A572078Hi | 57397 | 2080 |
| A572079Hi | 57398 | 2081 |
| A572080Hi | 57451 | 2082 |
| A572081Hi | 57451 | 2083 |
| A572082Hi | 57455 | 2084 |
| A572083Hi | 57458 | 2085 |
| A572084Hi | 57499 | 2086 |
| A572085Hi | 57500 | 2087 |
| 57501-58000 | | |
| A572086Hi | 57598 | 2088 |
| A572087Hi | 57673 | 2089 |
| A572088Hi | 57711 | 2090 |
| A572089Hi | 57738 | 2091 |
| A572090Hi | 57739 | 2092 |
| A572091Hi | 57917 | 2093 |
| 58001-58500 | | |
| A57131Hi | 58333 | 133 |
| A572092Hi | 58021 | 2094 |
| A572093Hi | 58072 | 2095 |
| A572094Hi | 58087 | 2096 |
| A572095Hi | 58089 | 2097 |
| A572096Hi | 58091 | 2098 |
| A572097Hi | 58099 | 2099 |
| A572098Hi | 58100 | 2100 |
| A572099Hi | 58130 | 2101 |
| A572100Hi | 58134 | 2102 |
| A572101Hi | 58334 | 2103 |
| A572102Hi | 58454 | 2104 |
| A572103Hi | 58497 | 2105 |
| A572104Hi | 58498 | 2106 |
| A572105Hi | 58499 | 2107 |
| 58501-59000 | | |
| A57132Hi | 58587 | 134 |
| A572106Hi | 58578 | 2108 |
| A572107Hi | 58580 | 2109 |
| A572108Hi | 58582 | 2110 |
| A572109Hi | 58583 | 2111 |
| A572110Hi | 58586 | 2112 |
| A572111Hi | 58587 | 2113 |
| A572112Hi | 58588 | 2114 |
| A572113Hi | 58597 | 2115 |
| A572114Hi | 58671 | 2116 |
| A572115Hi | 58674 | 2117 |
| A572116Hi | 58726 | 2118 |
| A572117Hi | 58770 | 2119 |
| A572118H | 58885 | 2120 |
| A572119H | 58890 | 2121 |
| A572120H | 58891 | 2122 |
| A572121H | 58891 | 2123 |
| A572122H | 58892 | 2124 |
| A572123H | 58893 | 2125 |
| A572124H | 58893 | 2126 |
| A572125H | 58895 | 2127 |
| A572126H | 58897 | 2128 |
| A572127H | 58898 | 2129 |
| A572128H | 58922 | 2130 |
| A572129H | 58922 | 2131 |
| A572130H | 58922 | 2132 |
| A572131H | 58923 | 2133 |
| A572132H | 58923 | 2134 |
| A572133H | 58924 | 2135 |
| A572134H | 58925 | 2136 |
| A572135H | 58925 | 2137 |
| A572136H | 58926 | 2138 |
| A572137H | 58926 | 2139 |
| A572138H | 58926 | 2140 |
| A572139H | 58927 | 2141 |
| A572140H | 58927 | 2142 |
| A572141H | 58928 | 2143 |
| A572142H | 58928 | 2144 |
| A572143H | 58928 | 2145 |
| A572144H | 58929 | 2146 |
| A572145H | 58930 | 2147 |
| A572146H | 58930 | 2148 |
| A572147H | 58931 | 2149 |
| A572148H | 58931 | 2150 |
| A572149H | 58932 | 2151 |
| A572150H | 58932 | 2152 |
| A572151H | 58933 | 2153 |
| A572152H | 58934 | 2154 |
| A572153H | 58934 | 2155 |
| A572154H | 58935 | 2156 |
| A572155H | 58935 | 2157 |
| A572156H | 58936 | 2158 |
| A572157H | 58939 | 2159 |
| A572158H | 58984 | 2160 |
| A572159H | 58984 | 2161 |
| A572160H | 58985 | 2162 |
| A572161H | 58985 | 2163 |
| A572162H | 58986 | 2164 |
| A572163H | 58987 | 2165 |
| A572164H | 58988 | 2166 |
| A572165H | 58992 | 2167 |
| A572166H | 58993 | 2168 |
| A572167H | 58995 | 2169 |
| A572168H | 58996 | 2170 |
| A572169H | 58996 | 2171 |
| A572170H | 58998 | 2172 |
| 59001-59500 | | |
| A57133Hi | 59248 | 135 |
| A572171H | 59004 | 2173 |
| A572172H | 59005 | 2174 |
| A572173H | 59007 | 2175 |
| A572174H | 59008 | 2176 |
| A572175H | 59008 | 2177 |
| A572176H | 59030 | 2178 |
| A572177H | 59031 | 2179 |
| A572178H | 59033 | 2180 |
| A572179H | 59060 | 2181 |
| A572180H | 59062 | 2182 |
| A572181H | 59063 | 2183 |
| A572182H | 59065 | 2184 |
| A572183H | 59066 | 2185 |
| A572184H | 59068 | 2186 |
| A572185H | 59070 | 2187 |
| A572186H | 59071 | 2188 |
| A572187H | 59078 | 2189 |
| A572188H | 59079 | 2190 |
| A572189H | 59088 | 2191 |
| A572190H | 59098 | 2192 |
| A572191H | 59099 | 2193 |
| A572192H | 59102 | 2194 |
| A572193H | 59110 | 2195 |
| A572194H | 59112 | 2196 |
| A572195Hi | 59141 | 2197 |
| A572196Hi | 59307 | 2198 |
| A572197Hi | 59308 | 2199 |
| A572198Hi | 59311 | 2200 |
| 59501-60000 | | |
| A572199Hi | 59519 | 2201 |
| A572200Hi | 59522 | 2202 |
| A572201Hi | 59523 | 2203 |
| A572202Hi | 59526 | 2204 |
| A572203Hi | 59529 | 2205 |
| A572204Hi | 59530 | 2206 |
| A572205Hi | 59604 | 2207 |
| A572206Hi | 59605 | 2208 |
| A572207Hi | 59666 | 2209 |
| A572208Hi | 59725 | 2210 |
| A572209Hi | 59913 | 2211 |
| A572210Hi | 59916 | 2212 |
| A572211Hi | 59985 | 2213 |
| A572212Hi | 59997 | 2214 |
| A572213Hi | 59998 | 2215 |
| A572214Hi | 59998 | 2216 |
| A572215Hi | 59999 | 2217 |
| A572216Hi | 60000 | 2218 |
| 60001-60500 | | |
| A572217Hi | 60001 | 2219 |
| A572218Hi | 60004 | 2220 |
| A572219Hi | 60005 | 2221 |
| A572220Hi | 60006 | 2222 |
| A572221Hi | 60064 | 2223 |
| A572222Hi | 60066 | 2224 |
| A572223Hi | 60137 | 2225 |
| A572224Hi | 60142 | 2226 |
| A572225Hi | 60245 | 2227 |
| A572226Hi | 60247 | 2228 |
| A572227Hi | 60249 | 2229 |
| A572228Hi | 60307 | 2230 |
| A572229Hi | 60310 | 2231 |
| A572230Hi | 60311 | 2232 |
| A572231Hi | 60341 | 2233 |
| A572232Hi | 60349 | 2234 |
| 60501-61000 | | |
| A57134Hi | 60574 | 136 |
| A572233Hi | 60575 | 2235 |
| A572234Hi | 60576 | 2236 |
| A572235Hi | 60599 | 2237 |
| A572236Hi | 60600 | 2238 |
| A572237Hi | 60742 | 2239 |
| A572238Hi | 60742 | 2240 |
| A572239Hi | 60743 | 2241 |
| A572240Hi | 60947 | 2242 |
| A572241Hi | 60981 | 2243 |
| A572242Hi | 60982 | 2244 |
| A572243Hi | 60983 | 2245 |
| 61001-61500 | | |
| A57135Hi | 61211 | 137 |
| A572244Hi | 61121 | 2246 |
| A572245Hi | 61205 | 2247 |
| A572246Hi | 61206 | 2248 |
| A572247Hi | 61209 | 2249 |
| A572248Hi | 61210 | 2250 |
| A572249Hi | 61211 | 2251 |
| A572250Hi | 61450 | 2252 |
| A572251Hi | 61451 | 2253 |
| A572252Hi | 61451 | 2254 |
| 61501-62000 | | |
| A57136Hi | 61793 | 138 |
| A572253Hi | 61638 | 2255 |
| A572254Hi | 61641 | 2256 |
| A572255Hi | 61647 | 2257 |
| A572256Hi | 61648 | 2258 |
| A572257Hi | 61671 | 2259 |
| A572258Hi | 61734 | 2260 |
| A572259Hi | 61788 | 2261 |
| A572260Hi | 61840 | 2262 |
| A572261Hi | 61843 | 2263 |
| A572262Hi | 61845 | 2264 |
| A572263Hi | 61847 | 2265 |
| A572264Hi | 61857 | 2266 |
| 62001-62500 | | |
| A572265Hi | 62035 | 2267 |
| A572266Hi | 62047 | 2268 |
| A572267Hi | 62308 | 2269 |
| A572268Hi | 62309 | 2270 |
| A572269H | 62355 | 2271 |
| A572270H | 62356 | 2272 |
| A572271H | 62357 | 2273 |
| A572272H | 62358 | 2274 |
| A572273H | 62359 | 2275 |
| A572274H | 62360 | 2276 |
| A572275H | 62360 | 2277 |
| A572276H | 62366 | 2278 |
| A572277H | 62367 | 2279 |
| A572278H | 62367 | 2280 |
| A572279H | 62368 | 2281 |
| A572280H | 62370 | 2282 |
| A572281H | 62371 | 2283 |
| A572282H | 62372 | 2284 |
| A572283H | 62372 | 2285 |
| A572284H | 62375 | 2286 |
| A572285H | 62376 | 2287 |
| A572286H | 62378 | 2288 |
| A572287H | 62380 | 2289 |
| A572288H | 62383 | 2290 |
| A572289H | 62386 | 2291 |
| A572290H | 62448 | 2292 |
| A572291H | 62449 | 2293 |
| A572292H | 62452 | 2294 |
| A572293H | 62453 | 2295 |
| A572294H | 62453 | 2296 |
| A572295H | 62455 | 2297 |
| A572296H | 62456 | 2298 |
| A572297H | 62456 | 2299 |
| A572298H | 62464 | 2300 |
| A572299H | 62465 | 2301 |
| A572300H | 62485 | 2302 |
| 62501-63000 | | |
| A572301Hi | 62778 | 2303 |
| A572302Hi | 62780 | 2304 |
| A572303Hi | 62781 | 2305 |
| A572304Hi | 62787 | 2306 |
| A572305Hi | 62789 | 2307 |
| A572306Hi | 62791 | 2308 |
| 63001-63500 | | |
| A57137Hi | 63162 | 139 |
| A572307Hi | 63129 | 2309 |
| A572308Hi | 63160 | 2310 |
| A572309Hi | 63161 | 2311 |
| A572310Hi | 63209 | 2312 |
| A572311Hi | 63210 | 2313 |
| A572312Hi | 63210 | 2314 |
| A572313Hi | 63211 | 2315 |
| A572314Hi | 63390 | 2316 |
| A572315Hi | 63445 | 2317 |
| 63501-64000 | | |
| A572316Hi | 63803 | 2318 |
| A572317Hi | 63807 | 2319 |
| A572318Hi | 63807 | 2320 |
| A572319Hi | 63809 | 2321 |
| A572320Hi | 63810 | 2322 |
| A572321Hi | 63990 | 2323 |
| 64001-64500 | | |
| A57138Hi | 64255 | 140 |
| A572322Hi | 64177 | 2324 |
| A572323Hi | 64179 | 2325 |
| A572324Hi | 64212 | 2326 |
| A572325Hi | 64213 | 2327 |
| A572326Hi | 64213 | 2328 |
| A572327Hi | 64215 | 2329 |
| A572328Hi | 64217 | 2330 |
| A572329Hi | 64217 | 2331 |
| A572330Hi | 64264 | 2332 |
| A572331Hi | 64345 | 2333 |
| 64501-65000 | | |
| A57139Hi | 64669 | 141 |
| A572332Hi | 64627 | 2334 |
| A572333Hi | 64661 | 2335 |
| A572334Hi | 64662 | 2336 |
| A572335Hi | 64662 | 2337 |
| A572336Hi | 64666 | 2338 |
| A572337Hi | 64822 | 2339 |
| A572338Hi | 64822 | 2340 |
| A572339Hi | 64823 | 2341 |
| A572340Hi | 64824 | 2342 |
| 65001-65500 | | |
| A572341Hi | 65006 | 2343 |
| A572342Hi | 65040 | 2344 |
| A572343Hi | 65042 | 2345 |
| A572344Hi | 65043 | 2346 |
| A572345Hi | 65164 | 2347 |
| A572346Hi | 65170 | 2348 |
| A572347Hi | 65279 | 2349 |
| A572348Hi | 65280 | 2350 |
| A572349Hi | 65280 | 2351 |
| A572350Hi | 65347 | 2352 |
| A572351Hi | 65494 | 2353 |
| A572352Hi | 65496 | 2354 |
| A572353Hi | 65500 | 2355 |
| 65501-66000 | | |
| A572354Hi | 65501 | 2356 |
| A572355Hi | 65556 | 2357 |
| A572356Hi | 65565 | 2358 |
| A572357Hi | 65566 | 2359 |
| A572358Hi | 65629 | 2360 |
| A572359Hi | 65705 | 2361 |
| A572360Hi | 65711 | 2362 |
| A572361Hi | 65714 | 2363 |
| A572362Hi | 65870 | 2364 |
| 66001-66500 | | |
| A572363Hi | 66490 | 2365 |
| 66501-67000 | | |
| A572364Hi | 66589 | 2366 |
| A572365Hi | 66709 | 2367 |
| A572366Hi | 66709 | 2368 |
| A572367Hi | 66719 | 2369 |
| A572368Hi | 66805 | 2370 |
| A572369Hi | 66806 | 2371 |
| A572370Hi | 66807 | 2372 |
| A572371Hi | 66808 | 2373 |
| A572372Hi | 66814 | 2374 |
| A572373Hi | 66848 | 2375 |
| A572374Hi | 66970 | 2376 |
| 67001-67500 | | |
| A57140Hi | 67470 | 142 |
| A575315Hi | 67470 | 142 |
| A575316Hi | 67470 | 142 |
| A575318Hi | 67470 | 142 |
| A575321Hi | 67470 | 142 |
| A575322Hi | 67470 | 142 |
| A575323Hi | 67470 | 142 |
| A575324Hi | 67470 | 142 |
| A575326Hi | 67470 | 142 |
| A575327Hi | 67470 | 142 |
| A572375Hi | 67129 | 2377 |
| A572376Hi | 67457 | 2378 |
| A572377Hi | 67471 | 2379 |
| A572378Hi | 67471 | 2380 |
| A572379Hi | 67472 | 2381 |
| 67501-68000 | | |
| A57141Hi | 67740 | 143 |
| A572380Hi | 67654 | 2382 |
| A572381Hi | 67655 | 2383 |
| A572382Hi | 67899 | 2384 |
| A572383Hi | 67902 | 2385 |
| A572384Hi | 67924 | 2386 |
| A572385Hi | 67946 | 2387 |
| 68001-68500 | | |
| A57142Hi | 68485 | 144 |
| A572386Hi | 68001 | 2388 |
| A572387Hi | 68003 | 2389 |
| A572388Hi | 68125 | 2390 |
| A572389Hi | 68234 | 2391 |
| A572390Hi | 68298 | 2392 |
| A572391Hi | 68483 | 2393 |
| 68501-69000 | | |
| A572392H | 68554 | 2394 |
| A572393H | 68555 | 2395 |
| A572394H | 68556 | 2396 |
| A572395H | 68556 | 2397 |
| A572396H | 68557 | 2398 |
| A572397H | 68558 | 2399 |
| A572398H | 68587 | 2400 |
| A572399H | 68588 | 2401 |
| A572400H | 68588 | 2402 |
| A572401H | 68594 | 2403 |
| A572402H | 68595 | 2404 |
| A572403H | 68595 | 2405 |
| A572404H | 68596 | 2406 |
| A572405H | 68596 | 2407 |
| A572406H | 68597 | 2408 |
| A572407H | 68597 | 2409 |
| A572408H | 68598 | 2410 |
| A572409H | 68602 | 2411 |
| A572410H | 68603 | 2412 |
| A572411H | 68603 | 2413 |
| A572412H | 68605 | 2414 |
| A572413H | 68606 | 2415 |
| A572414H | 68606 | 2416 |
| A572415H | 68607 | 2417 |
| A572416H | 68607 | 2418 |
| A572417H | 68607 | 2419 |
| A572418H | 68608 | 2420 |
| A572419H | 68608 | 2421 |
| A572420H | 68608 | 2422 |
| A572421H | 68608 | 2423 |
| A572422H | 68609 | 2424 |
| A572423H | 68609 | 2425 |
| A572424H | 68609 | 2426 |
| A572425H | 68610 | 2427 |
| A572426H | 68610 | 2428 |
| A572427H | 68610 | 2429 |
| A572428H | 68629 | 2430 |
| A572429H | 68629 | 2431 |
| A572430H | 68633 | 2432 |
| A572431Hi | 68710 | 2433 |
| A572432Hi | 68711 | 2434 |
| A572433Hi | 68712 | 2435 |
| A572434Hi | 68731 | 2436 |
| A572435Hi | 68737 | 2437 |
| A572436Hi | 68738 | 2438 |
| A572437Hi | 68739 | 2439 |
| A572438Hi | 68739 | 2440 |
| A572439Hi | 68743 | 2441 |
| A572440Hi | 68924 | 2442 |
| A572441Hi | 68924 | 2443 |
| 69001-69500 | | |
| A57143Hi | 69023 | 145 |
| A572442Hi | 69018 | 2444 |
| A572443Hi | 69024 | 2445 |
| A572444Hi | 69047 | 2446 |
| A572445Hi | 69048 | 2447 |
| A572446Hi | 69051 | 2448 |
| A572447Hi | 69055 | 2449 |
| A572448Hi | 69069 | 2450 |
| A572449Hi | 69082 | 2451 |
| A572450Hi | 69083 | 2452 |
| A572451Hi | 69087 | 2453 |
| A572452Hi | 69098 | 2454 |
| A572453Hi | 69100 | 2455 |
| A572454Hi | 69353 | 2456 |
| 69501-70000 | | |
| A572455Hi | 69551 | 2457 |
| A572456Hi | 69552 | 2458 |
| A572457H | 69849 | 2459 |
| A572458H | 69849 | 2460 |
| A572459H | 69850 | 2461 |
| A572460H | 69858 | 2462 |
| A572461H | 69860 | 2463 |
| A572462H | 69861 | 2464 |
| A572463H | 69894 | 2465 |
| A572464H | 69895 | 2466 |
| A572465H | 69896 | 2467 |
| A572466H | 69897 | 2468 |
| A572467H | 69898 | 2469 |
| A572468H | 69899 | 2470 |
| A572469H | 69900 | 2471 |
| A572470H | 69900 | 2472 |
| A572471Hi | 69921 | 2473 |
| A572472Hi | 69924 | 2474 |
| A572473Hi | 69925 | 2475 |
| A572474Hi | 69984 | 2476 |
| 70001-70500 | | |
| A572475Hi | 70104 | 2477 |
| A572476Hi | 70288 | 2478 |
| A572477Hi | 70289 | 2479 |
| A572478Hi | 70333 | 2480 |
| A572479Hi | 70334 | 2481 |
| A572480Hi | 70337 | 2482 |
| A572481Hi | 70339 | 2483 |
| A572482Hi | 70340 | 2484 |
| A572483Hi | 70344 | 2485 |
| A572484Hi | 70377 | 2486 |
| A572485H | 70431 | 2487 |
| A572486H | 70432 | 2488 |
| A572487H | 70432 | 2489 |
| A572488H | 70433 | 2490 |
| A572489H | 70435 | 2491 |
| A572490H | 70436 | 2492 |
| A572491H | 70437 | 2493 |
| A572492H | 70438 | 2494 |
| A572493H | 70442 | 2495 |
| A572494H | 70443 | 2496 |
| A572495H | 70443 | 2497 |
| A572496H | 70444 | 2498 |
| A572497H | 70444 | 2499 |
| A572498H | 70445 | 2500 |
| A572499H | 70446 | 2501 |
| A572500H | 70447 | 2502 |
| A572501H | 70447 | 2503 |
| A572502H | 70449 | 2504 |
| A572503H | 70450 | 2505 |
| A572504H | 70451 | 2506 |
| A572505H | 70452 | 2507 |
| A572506H | 70452 | 2508 |
| A572507H | 70453 | 2509 |
| A572508H | 70453 | 2510 |
| A572509H | 70456 | 2511 |
| A572510H | 70482 | 2512 |
| A572511H | 70492 | 2513 |
| A572512H | 70493 | 2514 |
| A572513H | 70495 | 2515 |
| A572514H | 70499 | 2516 |
| 70501-71000 | | |
| A57144Hi | 70797 | 146 |
| A57145Hi | 70984 | 147 |
| A57146Hi | 70996 | 148 |
| A572515H | 70501 | 2517 |
| A572516H | 70502 | 2518 |
| A572517H | 70503 | 2519 |
| A572518H | 70504 | 2520 |
| A572519H | 70504 | 2521 |
| A572520H | 70505 | 2522 |
| A572521H | 70507 | 2523 |
| A572522H | 70511 | 2524 |
| A572523H | 70515 | 2525 |
| A572524H | 70530 | 2526 |
| A572525H | 70531 | 2527 |
| A572526H | 70531 | 2528 |
| A572527H | 70532 | 2529 |
| A572528H | 70533 | 2530 |
| A572529H | 70534 | 2531 |
| A572530H | 70535 | 2532 |
| A572531H | 70537 | 2533 |
| A572532H | 70538 | 2534 |
| A572533H | 70538 | 2535 |
| A572534H | 70539 | 2536 |
| A572535H | 70539 | 2537 |
| A572536H | 70540 | 2538 |
| A572537H | 70540 | 2539 |
| A572538H | 70541 | 2540 |
| A572539H | 70541 | 2541 |
| A572540H | 70542 | 2542 |
| A572541H | 70543 | 2543 |
| A572542H | 70544 | 2544 |
| A572543H | 70577 | 2545 |
| A572544H | 70586 | 2546 |
| A572545H | 70587 | 2547 |
| A572546H | 70591 | 2548 |
| A572547H | 70592 | 2549 |
| A572548H | 70593 | 2550 |
| A572549H | 70593 | 2551 |
| A572550H | 70599 | 2552 |
| A572551H | 70600 | 2553 |
| A572552H | 70600 | 2554 |
| A572553H | 70601 | 2555 |
| A572554H | 70601 | 2556 |
| A572555H | 70602 | 2557 |
| A572556H | 70603 | 2558 |
| A572557H | 70603 | 2559 |
| A572558H | 70604 | 2560 |
| A572559H | 70604 | 2561 |
| A572560H | 70606 | 2562 |
| A572561H | 70607 | 2563 |
| A572562H | 70607 | 2564 |
| A572563H | 70608 | 2565 |
| A572564H | 70608 | 2566 |
| A572565H | 70609 | 2567 |
| A572566H | 70609 | 2568 |
| A572567H | 70609 | 2569 |
| A572568H | 70610 | 2570 |
| A572569H | 70610 | 2571 |
| A572570H | 70611 | 2572 |
| A572571H | 70611 | 2573 |
| A572572H | 70612 | 2574 |
| A572573H | 70612 | 2575 |
| A572574H | 70613 | 2576 |
| A572575H | 70614 | 2577 |
| A572576H | 70614 | 2578 |
| A572577H | 70616 | 2579 |
| A572578H | 70617 | 2580 |
| A572579H | 70618 | 2581 |
| A572580H | 70618 | 2582 |
| A572581H | 70619 | 2583 |
| A572582H | 70619 | 2584 |
| A572583H | 70620 | 2585 |
| A572584H | 70620 | 2586 |
| A572585H | 70621 | 2587 |
| A572586H | 70621 | 2588 |
| A572587H | 70622 | 2589 |
| A572588H | 70622 | 2590 |
| A572589H | 70622 | 2591 |
| A572590H | 70623 | 2592 |
| A572591H | 70623 | 2593 |
| A572592H | 70623 | 2594 |
| A572593H | 70624 | 2595 |
| A572594H | 70625 | 2596 |
| A572595H | 70627 | 2597 |
| A572596H | 70628 | 2598 |
| A572597Hi | 70788 | 2599 |
| A572598Hi | 70792 | 2600 |
| A572599Hi | 70846 | 2601 |
| A572600Hi | 70967 | 2602 |
| A572601Hi | 70969 | 2603 |
| A572602Hi | 70983 | 2604 |
| A572603Hi | 70985 | 2605 |
| A572604Hi | 70985 | 2606 |
| A572605Hi | 70986 | 2607 |
| A572606Hi | 70987 | 2608 |
| A572607Hi | 70993 | 2609 |
| A572608Hi | 70994 | 2610 |
| A572609Hi | 70995 | 2611 |
| A572610Hi | 70995 | 2612 |
| 71001-71500 | | |
| A57147Hi | 71257 | 149 |
| A572611Hi | 71005 | 2613 |
| A572612Hi | 71091 | 2614 |
| A572613Hi | 71208 | 2615 |
| A572614Hi | 71209 | 2616 |
| A572615Hi | 71209 | 2617 |
| A572616Hi | 71210 | 2618 |
| A572617Hi | 71210 | 2619 |
| A572618Hi | 71212 | 2620 |
| A572619Hi | 71213 | 2621 |
| A572620Hi | 71218 | 2622 |
| A572621Hi | 71283 | 2623 |
| 71501-72000 | | |
| A572622Hi | 71645 | 2624 |
| A572623Hi | 71646 | 2625 |
| A572624Hi | 71650 | 2626 |
| A572625Hi | 71865 | 2627 |
| A572626Hi | 71866 | 2628 |
| A572627Hi | 71867 | 2629 |
| A572628Hi | 71868 | 2630 |
| A572629Hi | 71911 | 2631 |
| 72001-72500 | | |
| A57148Hi | 72080 | 150 |
| A572630Hi | 72022 | 2632 |
| A572631Hi | 72093 | 2633 |
| A572632Hi | 72095 | 2634 |
| A572633Hi | 72158 | 2635 |
| A572634Hi | 72158 | 2636 |
| A572635Hi | 72304 | 2637 |
| A572636Hi | 72305 | 2638 |
| A572637Hi | 72309 | 2639 |
| A572638Hi | 72316 | 2640 |
| A572639Hi | 72318 | 2641 |
| A572640Hi | 72319 | 2642 |
| A572641Hi | 72321 | 2643 |
| A572642Hi | 72321 | 2644 |
| A572643Hi | 72322 | 2645 |
| A572644Hi | 72353 | 2646 |
| 72501-73000 | | |
| A572645Hi | 72988 | 2647 |
| A572646Hi | 72989 | 2648 |
| A572647Hi | 72990 | 2649 |
| A572648Hi | 72991 | 2650 |
| A572649Hi | 72992 | 2651 |
| A572650Hi | 72994 | 2652 |
| A572651Hi | 72994 | 2653 |
| 73001-73500 | | |
| A572652Hi | 73097 | 2654 |
| A572653Hi | 73142 | 2655 |
| A572654Hi | 73142 | 2656 |
| A572655Hi | 73147 | 2657 |
| A572656Hi | 73148 | 2658 |
| A572657Hi | 73148 | 2659 |
| A572658Hi | 73153 | 2660 |
| A572659Hi | 73160 | 2661 |
| A572660Hi | 73160 | 2662 |
| A572661Hi | 73161 | 2663 |
| A572662Hi | 73170 | 2664 |
| A572663Hi | 73177 | 2665 |
| A572664Hi | 73179 | 2666 |
| A572665Hi | 73189 | 2667 |
| A572666H | 73255 | 2668 |
| A572667H | 73256 | 2669 |
| A572668H | 73257 | 2670 |
| A572669H | 73263 | 2671 |
| A572670H | 73268 | 2672 |
| A572671H | 73271 | 2673 |
| A572672H | 73272 | 2674 |
| A572673H | 73277 | 2675 |
| A572674H | 73279 | 2676 |
| A572675H | 73281 | 2677 |
| A572676H | 73337 | 2678 |
| A572677H | 73338 | 2679 |
| A572678H | 73338 | 2680 |
| A572679H | 73339 | 2681 |
| A572680H | 73342 | 2682 |
| A572681H | 73343 | 2683 |
| A572682H | 73343 | 2684 |
| A572683H | 73345 | 2685 |
| A572684H | 73346 | 2686 |
| A572685H | 73346 | 2687 |
| A572686H | 73347 | 2688 |
| A572687H | 73347 | 2689 |
| A572688H | 73348 | 2690 |
| A572689H | 73348 | 2691 |
| A572690H | 73349 | 2692 |
| A572691H | 73353 | 2693 |
| A572692H | 73354 | 2694 |
| A572693H | 73356 | 2695 |
| A572694H | 73357 | 2696 |
| A572695H | 73358 | 2697 |
| A572696H | 73358 | 2698 |
| A572697H | 73363 | 2699 |
| A572698H | 73365 | 2700 |
| A572699H | 73388 | 2701 |
| A572700H | 73393 | 2702 |
| A572701H | 73394 | 2703 |
| A572702H | 73395 | 2704 |
| A572703H | 73397 | 2705 |
| A572704H | 73400 | 2706 |
| A572705H | 73405 | 2707 |
| A572706H | 73407 | 2708 |
| A572707H | 73408 | 2709 |
| A572708H | 73409 | 2710 |
| A572709H | 73410 | 2711 |
| A572710H | 73411 | 2712 |
| A572711H | 73414 | 2713 |
| A572712H | 73415 | 2714 |
| A572713H | 73415 | 2715 |
| A572714H | 73416 | 2716 |
| A572715H | 73416 | 2717 |
| A572716H | 73475 | 2718 |
| A572717H | 73477 | 2719 |
| A572718H | 73478 | 2720 |
| 73501-74000 | | |
| A572719Hi | 73529 | 2721 |
| A572720Hi | 73996 | 2722 |
| A572721Hi | 73998 | 2723 |
| 74001-74500 | | |
| A572722Hi | 74088 | 2724 |
| A572723Hi | 74089 | 2725 |
| A572724H | 74118 | 2726 |
| A572725H | 74119 | 2727 |
| A572726H | 74124 | 2728 |
| A572727H | 74125 | 2729 |
| A572728H | 74125 | 2730 |
| A572729H | 74126 | 2731 |
| A572730H | 74126 | 2732 |
| A572731H | 74127 | 2733 |
| A572732H | 74127 | 2734 |
| A572733H | 74128 | 2735 |
| A572734H | 74128 | 2736 |
| A572735H | 74129 | 2737 |
| A572736H | 74129 | 2738 |
| A572737H | 74130 | 2739 |
| A572738H | 74131 | 2740 |
| A572739H | 74131 | 2741 |
| A572740H | 74132 | 2742 |
| A572741H | 74132 | 2743 |
| A572742H | 74133 | 2744 |
| A572743H | 74133 | 2745 |
| A572744H | 74134 | 2746 |
| A572745H | 74135 | 2747 |
| A572746H | 74137 | 2748 |
| A572747H | 74138 | 2749 |
| A572748H | 74139 | 2750 |
| A572749H | 74140 | 2751 |
| A572750H | 74160 | 2752 |
| A572751H | 74184 | 2753 |
| A572752H | 74186 | 2754 |
| A572753H | 74193 | 2755 |
| A572754H | 74200 | 2756 |
| A572755H | 74201 | 2757 |
| A572756H | 74204 | 2758 |
| A572757H | 74205 | 2759 |
| A572758H | 74206 | 2760 |
| A572759H | 74210 | 2761 |
| A572760H | 74211 | 2762 |
| A572761H | 74212 | 2763 |
| A572762H | 74212 | 2764 |
| A572763H | 74213 | 2765 |
| A572764H | 74214 | 2766 |
| A572765H | 74215 | 2767 |
| A572766H | 74217 | 2768 |
| A572767H | 74218 | 2769 |
| A572768H | 74218 | 2770 |
| A572769H | 74219 | 2771 |
| A572770H | 74220 | 2772 |
| A572771H | 74221 | 2773 |
| A572772H | 74222 | 2774 |
| A572773H | 74223 | 2775 |
| A572774H | 74226 | 2776 |
| A572775H | 74227 | 2777 |
| A572776H | 74229 | 2778 |
| A572777H | 74230 | 2779 |
| A572778Hi | 74278 | 2780 |
| A572779Hi | 74278 | 2781 |
| A572780Hi | 74297 | 2782 |
| A572781Hi | 74297 | 2783 |
| A572782Hi | 74298 | 2784 |
| A572783Hi | 74302 | 2785 |
| A572784Hi | 74360 | 2786 |
| A572785Hi | 74361 | 2787 |
| A572786Hi | 74363 | 2788 |
| 74501-75000 | | |
| A572787Hi | 74753 | 2789 |
| A572788Hi | 74776 | 2790 |
| A572789Hi | 74777 | 2791 |
| A572790Hi | 74779 | 2792 |
| A572791Hi | 74786 | 2793 |
| A572792Hi | 74970 | 2794 |
| A572793Hi | 74976 | 2795 |
| A572794Hi | 74977 | 2796 |
| A572795Hi | 74978 | 2797 |
| A572796Hi | 74978 | 2798 |
| A572797Hi | 74979 | 2799 |
| A572798Hi | 74979 | 2800 |
| 75001-75500 | | |
| A57149Hi | 75451 | 151 |
| A572799Hi | 75088 | 2801 |
| A572800Hi | 75212 | 2802 |
| A572801Hi | 75304 | 2803 |
| A572802Hi | 75317 | 2804 |
| A572803Hi | 75318 | 2805 |
| A572804Hi | 75400 | 2806 |
| A572805Hi | 75401 | 2807 |
| A572806Hi | 75402 | 2808 |
| A572807Hi | 75407 | 2809 |
| A572808Hi | 75448 | 2810 |
| A572809Hi | 75476 | 2811 |
| A572810Hi | 75477 | 2812 |
| 75501-76000 | | |
| A57150Hi | 75543 | 152 |
| A57151Hi | 75551 | 153 |
| A57152Hi | 75722 | 154 |
| A57153Hi | 75814 | 155 |
| A572811Hi | 75546 | 2813 |
| A572812Hi | 75548 | 2814 |
| A572813Hi | 75549 | 2815 |
| A572814Hi | 75553 | 2816 |
| A572815Hi | 75559 | 2817 |
| A572816Hi | 75560 | 2818 |
| A572817Hi | 75688 | 2819 |
| A572818Hi | 75723 | 2820 |
| A572819Hi | 75724 | 2821 |
| A572820Hi | 75725 | 2822 |
| A572821Hi | 75726 | 2823 |
| A572822Hi | 75747 | 2824 |
| A572823Hi | 75752 | 2825 |
| A572824Hi | 75754 | 2826 |
| A572825Hi | 75818 | 2827 |
| A572826Hi | 75824 | 2828 |
| A572827Hi | 75849 | 2829 |
| A572828Hi | 75935 | 2830 |
| A572829Hi | 75938 | 2831 |
| A572830Hi | 75939 | 2832 |
| A572831Hi | 75941 | 2833 |
| A572832Hi | 75942 | 2834 |
| A572833Hi | 75946 | 2835 |
| A572834Hi | 75960 | 2836 |
| A572835Hi | 75968 | 2837 |
| A572836Hi | 75973 | 2838 |
| A572837Hi | 75974 | 2839 |
| A572838Hi | 75975 | 2840 |
| A572839Hi | 75975 | 2841 |
| A572840Hi | 75998 | 2842 |
| A572841Hi | 75999 | 2843 |
| 76001-76500 | | |
| A57154Hi | 76162 | 156 |
| A572842Hi | 76123 | 2844 |
| A572843Hi | 76124 | 2845 |
| A572844Hi | 76127 | 2846 |
| A572845Hi | 76168 | 2847 |
| A572846Hi | 76169 | 2848 |
| 76501-77000 | | |
| A57155Hi | 76526 | 157 |
| A57156Hi | 76645 | 158 |
| A572847Hi | 76505 | 2849 |
| A572848Hi | 76509 | 2850 |
| A572849Hi | 76510 | 2851 |
| A572850Hi | 76511 | 2852 |
| A572851Hi | 76514 | 2853 |
| A572852Hi | 76515 | 2854 |
| A572853Hi | 76516 | 2855 |
| A572854Hi | 76518 | 2856 |
| A572855Hi | 76525 | 2857 |
| A572856Hi | 76527 | 2858 |
| A572857Hi | 76528 | 2859 |
| A572858Hi | 76529 | 2860 |
| A572859Hi | 76596 | 2861 |
| A572860Hi | 76597 | 2862 |
| A572861Hi | 76597 | 2863 |
| A572862Hi | 76598 | 2864 |
| A572863Hi | 76599 | 2865 |
| A572864Hi | 76642 | 2866 |
| A572865Hi | 76644 | 2867 |
| A572866Hi | 76646 | 2868 |
| A572867Hi | 76853 | 2869 |
| A572868Hi | 76853 | 2870 |
| A572869Hi | 76854 | 2871 |
| A572870Hi | 76861 | 2872 |
| A572871Hi | 76868 | 2873 |
| 77001-77500 | | |
| A572872Hi | 77334 | 2874 |
| A572873Hi | 77343 | 2875 |
| A572874Hi | 77344 | 2876 |
| A572875Hi | 77345 | 2877 |
| A572876Hi | 77346 | 2878 |
| A572877Hi | 77346 | 2879 |
| A572878Hi | 77351 | 2880 |
| A572879Hi | 77479 | 2881 |
| 77501-78000 | | |
| A57157Hi | 77928 | 159 |
| A572880Hi | 77520 | 2882 |
| A572881Hi | 77556 | 2883 |
| A572882Hi | 77561 | 2884 |
| A572883Hi | 77613 | 2885 |
| A572884Hi | 77670 | 2886 |
| A572885H | 77793 | 2887 |
| A572886H | 77793 | 2888 |
| A572887H | 77795 | 2889 |
| A572888H | 77856 | 2890 |
| A572889Hi | 77925 | 2891 |
| A572890Hi | 77934 | 2892 |
| 78001-78500 | | |
| A57158Hi | 78143 | 160 |
| A572891Hi | 78078 | 2893 |
| A572892Hi | 78078 | 2894 |
| A572893Hi | 78144 | 2895 |
| A572894Hi | 78145 | 2896 |
| A572895Hi | 78146 | 2897 |
| A572896Hi | 78148 | 2898 |
| A572897Hi | 78148 | 2899 |
| A572898Hi | 78153 | 2900 |
| A572899Hi | 78153 | 2901 |
| A572900Hi | 78273 | 2902 |
| A572901Hi | 78275 | 2903 |
| A572902Hi | 78281 | 2904 |
| A572903Hi | 78410 | 2905 |
| A572904Hi | 78416 | 2906 |
| A572905Hi | 78469 | 2907 |
| 78501-79000 | | |
| A572906Hi | 78523 | 2908 |
| A572907Hi | 78524 | 2909 |
| A572908Hi | 78534 | 2910 |
| A572909Hi | 78538 | 2911 |
| A572910Hi | 78542 | 2912 |
| A572911Hi | 78543 | 2913 |
| A572912Hi | 78591 | 2914 |
| A572913Hi | 78593 | 2915 |
| A572914Hi | 78594 | 2916 |
| A572915Hi | 78595 | 2917 |
| A572916Hi | 78599 | 2918 |
| A572917Hi | 78605 | 2919 |
| A572918Hi | 78792 | 2920 |
| A572919Hi | 78794 | 2921 |
| A572920Hi | 78795 | 2922 |
| A572921Hi | 78809 | 2923 |
| A572922Hi | 78823 | 2924 |
| A572923Hi | 78823 | 2925 |
| A572924Hi | 78826 | 2926 |
| A572925H | 78952 | 2927 |
| A572926H | 78952 | 2928 |
| A572927H | 78953 | 2929 |
| A572928H | 78953 | 2930 |
| A572929H | 78955 | 2931 |
| A572930H | 78956 | 2932 |
| A572931H | 78956 | 2933 |
| A572932H | 78957 | 2934 |
| A572933H | 78958 | 2935 |
| A572934H | 78958 | 2936 |
| A572935H | 78959 | 2937 |
| A572936H | 78960 | 2938 |
| A572937H | 78962 | 2939 |
| A572938H | 78964 | 2940 |
| 79001-79500 | | |
| A572939H | 79005 | 2941 |
| A572940H | 79006 | 2942 |
| A572941H | 79009 | 2943 |
| A572942H | 79010 | 2944 |
| A572943H | 79011 | 2945 |
| A572944H | 79012 | 2946 |
| A572945H | 79013 | 2947 |
| A572946H | 79015 | 2948 |
| A572947H | 79017 | 2949 |
| A572948H | 79018 | 2950 |
| A572949H | 79019 | 2951 |
| A572950H | 79020 | 2952 |
| A572951H | 79020 | 2953 |
| A572952H | 79021 | 2954 |
| A572953H | 79026 | 2955 |
| A572954H | 79035 | 2956 |
| A572955H | 79036 | 2957 |
| A572956H | 79037 | 2958 |
| A572957H | 79038 | 2959 |
| A572958H | 79038 | 2960 |
| A572959H | 79039 | 2961 |
| A572960H | 79040 | 2962 |
| A572961H | 79041 | 2963 |
| A572962H | 79042 | 2964 |
| A572963H | 79042 | 2965 |
| A572964H | 79043 | 2966 |
| A572965H | 79043 | 2967 |
| A572966H | 79044 | 2968 |
| A572967H | 79044 | 2969 |
| A572968H | 79045 | 2970 |
| A572969H | 79048 | 2971 |
| A572970H | 79049 | 2972 |
| A572971H | 79050 | 2973 |
| A572972H | 79051 | 2974 |
| A572973H | 79052 | 2975 |
| A572974H | 79053 | 2976 |
| A572975H | 79054 | 2977 |
| A572976H | 79054 | 2978 |
| A572977H | 79055 | 2979 |
| A572978H | 79056 | 2980 |
| A572979H | 79060 | 2981 |
| A572980H | 79061 | 2982 |
| A572981H | 79061 | 2983 |
| A572982H | 79062 | 2984 |
| A572983H | 79065 | 2985 |
| A572984H | 79085 | 2986 |
| A572985H | 79093 | 2987 |
| A572986H | 79095 | 2988 |
| A572987H | 79096 | 2989 |
| A572988H | 79097 | 2990 |
| A572989H | 79098 | 2991 |
| A572990H | 79106 | 2992 |
| A572991H | 79107 | 2993 |
| A572992H | 79109 | 2994 |
| A572993H | 79115 | 2995 |
| A572994H | 79119 | 2996 |
| A572995Hi | 79166 | 2997 |
| A572996Hi | 79167 | 2998 |
| A572997Hi | 79168 | 2999 |
| A572998Hi | 79233 | 3000 |
| A572999Hi | 79243 | 3001 |
| A573000Hi | 79247 | 3002 |
| A573001Hi | 79249 | 3003 |
| A573002Hi | 79455 | 3004 |
| A573003Hi | 79456 | 3005 |
| A573004Hi | 79457 | 3006 |
| A573005Hi | 79459 | 3007 |
| A573006Hi | 79460 | 3008 |
| A573007Hi | 79464 | 3009 |
| A573008Hi | 79464 | 3010 |
| A573009Hi | 79466 | 3011 |
| A573010Hi | 79493 | 3012 |
| A573011Hi | 79494 | 3013 |
| 79501-80000 | | |
| A57159Hi | 79662 | 161 |
| A57160Hi | 79809 | 162 |
| A573012Hi | 79546 | 3014 |
| A573013Hi | 79589 | 3015 |
| A573014Hi | 79594 | 3016 |
| A573015Hi | 79663 | 3017 |
| A573016Hi | 79665 | 3018 |
| A573017Hi | 79666 | 3019 |
| A573018Hi | 79668 | 3020 |
| A573019Hi | 79671 | 3021 |
| A573020Hi | 79672 | 3022 |
| A573021Hi | 79685 | 3023 |
| A573022Hi | 79735 | 3024 |
| A573023Hi | 79910 | 3025 |
| A573024Hi | 79911 | 3026 |
| A573025Hi | 79936 | 3027 |
| A573026Hi | 79938 | 3028 |
| A573027Hi | 79939 | 3029 |
| A573028Hi | 79942 | 3030 |
| A573029Hi | 79943 | 3031 |
| A573030Hi | 79943 | 3032 |
| A573031Hi | 79944 | 3033 |
| 80001-80500 | | |
| A573032Hi | 80030 | 3034 |
| A573033Hi | 80200 | 3035 |
| A573034Hi | 80385 | 3036 |
| A573035Hi | 80390 | 3037 |
| A573036Hi | 80424 | 3038 |
| A573037Hi | 80427 | 3039 |
| A573038Hi | 80428 | 3040 |
| A573039Hi | 80487 | 3041 |
| A573040Hi | 80488 | 3042 |
| 80501-81000 | | |
| A57161Hi | 80509 | 163 |
| A57162Hi | 80622 | 164 |
| A57163Hi | 80632 | 165 |
| A573041Hi | 80503 | 3043 |
| A573042Hi | 80508 | 3044 |
| A573043Hi | 80509 | 3045 |
| A573044Hi | 80616 | 3046 |
| A573045Hi | 80623 | 3047 |
| A573046Hi | 80624 | 3048 |
| A573047Hi | 80625 | 3049 |
| A573048Hi | 80627 | 3050 |
| A573049Hi | 80629 | 3051 |
| A573050Hi | 80634 | 3052 |
| A573051Hi | 80679 | 3053 |
| A573052Hi | 80680 | 3054 |
| A573053Hi | 80680 | 3055 |
| A573054Hi | 80681 | 3056 |
| A573055Hi | 80681 | 3057 |
| A573056Hi | 80683 | 3058 |
| A573057Hi | 80686 | 3059 |
| A573058Hi | 80687 | 3060 |
| A573059Hi | 80687 | 3061 |
| A573060Hi | 80692 | 3062 |
| A573061Hi | 80697 | 3063 |
| A573062Hi | 80698 | 3064 |
| A573063H | 80789 | 3065 |
| A573064H | 80790 | 3066 |
| A573065H | 80794 | 3067 |
| A573066H | 80795 | 3068 |
| A573067H | 80796 | 3069 |
| A573068H | 80798 | 3070 |
| A573069H | 80801 | 3071 |
| A573070H | 80802 | 3072 |
| A573071H | 80807 | 3073 |
| A573072H | 80808 | 3074 |
| A573073H | 80810 | 3075 |
| A573074H | 80820 | 3076 |
| A573075H | 80821 | 3077 |
| A573076H | 80826 | 3078 |
| A573077H | 80827 | 3079 |
| A573078H | 80830 | 3080 |
| A573079H | 80831 | 3081 |
| A573080H | 80831 | 3082 |
| A573081H | 80832 | 3083 |
| A573082H | 80832 | 3084 |
| A573083H | 80833 | 3085 |
| A573084H | 80834 | 3086 |
| A573085H | 80837 | 3087 |
| A573086H | 80841 | 3088 |
| A573087H | 80842 | 3089 |
| A573088H | 80844 | 3090 |
| A573089H | 80848 | 3091 |
| A573090H | 80852 | 3092 |
| A573091H | 80854 | 3093 |
| A573092H | 80855 | 3094 |
| A573093H | 80856 | 3095 |
| A573094H | 80856 | 3096 |
| A573095H | 80876 | 3097 |
| A573096H | 80881 | 3098 |
| A573097H | 80886 | 3099 |
| A573098H | 80894 | 3100 |
| A573099H | 80896 | 3101 |
| A573100H | 80897 | 3102 |
| A573101H | 80900 | 3103 |
| A573102H | 80933 | 3104 |
| A573103H | 80942 | 3105 |
| A573104H | 80943 | 3106 |
| A573105H | 80946 | 3107 |
| A573106H | 80948 | 3108 |
| A573107H | 80949 | 3109 |
| 81001-81500 | | |
| A573108Hi | 81026 | 3110 |
| A573109Hi | 81134 | 3111 |
| A573110Hi | 81135 | 3112 |
| A573111Hi | 81345 | 3113 |
| A573112Hi | 81416 | 3114 |
| A573113Hi | 81427 | 3115 |
| A573114Hi | 81428 | 3116 |
| A573115Hi | 81461 | 3117 |
| A573116Hi | 81461 | 3118 |
| A573117Hi | 81462 | 3119 |
| A573118Hi | 81470 | 3120 |
| A573119Hi | 81471 | 3121 |
| A573120Hi | 81472 | 3122 |
| A573121Hi | 81474 | 3123 |
| 81501-82000 | | |
| A573122Hi | 81560 | 3124 |
| A573123Hi | 81560 | 3125 |
| A573124Hi | 81563 | 3126 |
| A573125Hi | 81643 | 3127 |
| A573126Hi | 81647 | 3128 |
| A573127Hi | 81748 | 3129 |
| A573128Hi | 81984 | 3130 |
| A573129Hi | 81990 | 3131 |
| A573130Hi | 81991 | 3132 |
| 82001-82500 | | |
| A57164Hi | 82190 | 166 |
| A57165Hi | 82232 | 167 |
| 82501-83000 | | |
| A573131Hi | 82514 | 3133 |
| A573132Hi | 82717 | 3134 |
| A573133Hi | 82720 | 3135 |
| A573134Hi | 82720 | 3136 |
| 83001-83500 | | |
| A573135Hi | 83172 | 3137 |
| A573136Hi | 83173 | 3138 |
| A573137Hi | 83174 | 3139 |
| A573138Hi | 83177 | 3140 |
| A573139Hi | 83177 | 3141 |
| A573140Hi | 83178 | 3142 |
| A573141Hi | 83178 | 3143 |
| A573142Hi | 83302 | 3144 |
| A573143Hi | 83319 | 3145 |
| A573144Hi | 83320 | 3146 |
| A573145Hi | 83324 | 3147 |
| A573146Hi | 83400 | 3148 |
| 83501-84000 | | |
| A573147H | 83505 | 3149 |
| A573148H | 83505 | 3150 |
| A573149H | 83506 | 3151 |
| A573150H | 83507 | 3152 |
| A573151H | 83507 | 3153 |
| A573152H | 83534 | 3154 |
| A573153H | 83535 | 3155 |
| A573154H | 83535 | 3156 |
| A573155H | 83536 | 3157 |
| A573156H | 83537 | 3158 |
| A573157H | 83537 | 3159 |
| A573158H | 83538 | 3160 |
| A573159H | 83539 | 3161 |
| A573160H | 83540 | 3162 |
| A573161H | 83541 | 3163 |
| A573162H | 83541 | 3164 |
| A573163H | 83542 | 3165 |
| A573164H | 83542 | 3166 |
| A573165H | 83567 | 3167 |
| A573166H | 83568 | 3168 |
| A573167H | 83580 | 3169 |
| A573168H | 83587 | 3170 |
| A573169H | 83591 | 3171 |
| A573170H | 83593 | 3172 |
| A573171H | 83594 | 3173 |
| A573172H | 83595 | 3174 |
| A573173H | 83595 | 3175 |
| A573174H | 83604 | 3176 |
| A573175H | 83605 | 3177 |
| A573176H | 83606 | 3178 |
| A573177H | 83607 | 3179 |
| A573178H | 83608 | 3180 |
| A573179H | 83608 | 3181 |
| A573180H | 83609 | 3182 |
| A573181H | 83610 | 3183 |
| A573182H | 83611 | 3184 |
| A573183H | 83611 | 3185 |
| A573184H | 83612 | 3186 |
| A573185H | 83613 | 3187 |
| A573186H | 83613 | 3188 |
| A573187H | 83614 | 3189 |
| A573188H | 83621 | 3190 |
| A573189H | 83622 | 3191 |
| A573190H | 83622 | 3192 |
| A573191H | 83623 | 3193 |
| A573192H | 83624 | 3194 |
| A573193H | 83629 | 3195 |
| A573194H | 83630 | 3196 |
| A573195H | 83631 | 3197 |
| A573196H | 83631 | 3198 |
| A573197H | 83632 | 3199 |
| A573198H | 83633 | 3200 |
| A573199H | 83633 | 3201 |
| A573200H | 83634 | 3202 |
| A573201H | 83635 | 3203 |
| A573202H | 83635 | 3204 |
| A573203H | 83636 | 3205 |
| A573204H | 83636 | 3206 |
| A573205H | 83637 | 3207 |
| A573206H | 83637 | 3208 |
| A573207H | 83638 | 3209 |
| A573208H | 83659 | 3210 |
| A573209H | 83682 | 3211 |
| A573210Hi | 83775 | 3212 |
| A573211Hi | 83778 | 3213 |
| A573212Hi | 83779 | 3214 |
| A573213Hi | 83997 | 3215 |
| 84001-84500 | | |
| A57257H | 84139 | 259 |
| A57258H | 84140 | 260 |
| A57259H | 84141 | 261 |
| A57260H | 84142 | 262 |
| A573214H | 84139 | 3216 |
| A573215H | 84139 | 3217 |
| A573216H | 84140 | 3218 |
| A573217H | 84140 | 3219 |
| A573218H | 84141 | 3220 |
| A573219H | 84141 | 3221 |
| A573220H | 84141 | 3222 |
| A573221H | 84142 | 3223 |
| A573222H | 84142 | 3224 |
| A573223H | 84142 | 3225 |
| A573224H | 84143 | 3226 |
| A573225H | 84143 | 3227 |
| A573226H | 84143 | 3228 |
| A573227H | 84143 | 3229 |
| A573228H | 84144 | 3230 |
| A573229H | 84144 | 3231 |
| A573230H | 84145 | 3232 |
| A573231H | 84145 | 3233 |
| A573232H | 84145 | 3234 |
| A573233H | 84146 | 3235 |
| A573234H | 84146 | 3236 |
| A573235H | 84146 | 3237 |
| A573236H | 84147 | 3238 |
| A573237H | 84147 | 3239 |
| A573238H | 84148 | 3240 |
| A573239H | 84148 | 3241 |
| A573240H | 84148 | 3242 |
| A573241H | 84149 | 3243 |
| A573242H | 84149 | 3244 |
| A573243H | 84149 | 3245 |
| A573244H | 84149 | 3246 |
| A573245H | 84150 | 3247 |
| A573246H | 84150 | 3248 |
| A573247H | 84151 | 3249 |
| A573248H | 84151 | 3250 |
| A573249H | 84151 | 3251 |
| A573250H | 84152 | 3252 |
| A573251H | 84152 | 3253 |
| A573252H | 84152 | 3254 |
| A573253H | 84153 | 3255 |
| A573254H | 84154 | 3256 |
| A573255H | 84154 | 3257 |
| A573256H | 84155 | 3258 |
| A573257H | 84156 | 3259 |
| A573258H | 84157 | 3260 |
| A573259H | 84158 | 3261 |
| A573260H | 84162 | 3262 |
| A573261H | 84163 | 3263 |
| A573262H | 84164 | 3264 |
| A573263H | 84164 | 3265 |
| A573264H | 84165 | 3266 |
| A573265H | 84190 | 3267 |
| A573266H | 84190 | 3268 |
| A573267H | 84191 | 3269 |
| A573268H | 84192 | 3270 |
| A573269H | 84192 | 3271 |
| A573270H | 84193 | 3272 |
| A573271H | 84193 | 3273 |
| A573272H | 84194 | 3274 |
| A573273H | 84194 | 3275 |
| A573274H | 84195 | 3276 |
| A573275H | 84196 | 3277 |
| A573276H | 84197 | 3278 |
| A573277H | 84201 | 3279 |
| A573278H | 84202 | 3280 |
| A573279H | 84206 | 3281 |
| A573280H | 84207 | 3282 |
| A573281H | 84207 | 3283 |
| A573282H | 84208 | 3284 |
| A573283H | 84209 | 3285 |
| A573284H | 84210 | 3286 |
| A573285H | 84211 | 3287 |
| A573286H | 84211 | 3288 |
| A573287H | 84214 | 3289 |
| A573288H | 84217 | 3290 |
| A573289H | 84218 | 3291 |
| A573290H | 84218 | 3292 |
| A573291H | 84219 | 3293 |
| 84501-85000 | | |
| A573292Hi | 84643 | 3294 |
| A573293Hi | 84724 | 3295 |
| A573294Hi | 84795 | 3296 |
| A573295Hi | 84796 | 3297 |
| A573296Hi | 84797 | 3298 |
| A573297Hi | 84987 | 3299 |
| 85001-85500 | | |
| A573298Hi | 85076 | 3300 |
| A573299Hi | 85077 | 3301 |
| A573300Hi | 85077 | 3302 |
| A573301Hi | 85080 | 3303 |
| A573302Hi | 85345 | 3304 |
| A573303Hi | 85346 | 3305 |
| A573304Hi | 85346 | 3306 |
| A573305Hi | 85347 | 3307 |
| A573306Hi | 85348 | 3308 |
| A573307Hi | 85354 | 3309 |
| A573308Hi | 85365 | 3310 |
| A573309Hi | 85366 | 3311 |
| A573310Hi | 85367 | 3312 |
| A573311Hi | 85371 | 3313 |
| A573312H | 85435 | 3314 |
| A573313H | 85435 | 3315 |
| A573314H | 85436 | 3316 |
| A573315H | 85436 | 3317 |
| A573316H | 85437 | 3318 |
| A573317H | 85437 | 3319 |
| A573318H | 85437 | 3320 |
| A573319H | 85439 | 3321 |
| A573320H | 85441 | 3322 |
| A573321H | 85443 | 3323 |
| A573322H | 85449 | 3324 |
| A573323H | 85454 | 3325 |
| A573324H | 85455 | 3326 |
| A573325H | 85455 | 3327 |
| A573326H | 85456 | 3328 |
| A573327H | 85457 | 3329 |
| A573328H | 85457 | 3330 |
| A573329H | 85459 | 3331 |
| A573330H | 85460 | 3332 |
| A573331H | 85461 | 3333 |
| A573332H | 85461 | 3334 |
| A573333H | 85462 | 3335 |
| A573334H | 85462 | 3336 |
| A573335H | 85463 | 3337 |
| A573336H | 85463 | 3338 |
| A573337H | 85464 | 3339 |
| A573338H | 85464 | 3340 |
| 85501-86000 | | |
| A573339H | 85517 | 3341 |
| A573340H | 85519 | 3342 |
| A573341H | 85522 | 3343 |
| A573342H | 85524 | 3344 |
| A573343H | 85528 | 3345 |
| A573344H | 85529 | 3346 |
| A573345H | 85530 | 3347 |
| A573346H | 85530 | 3348 |
| A573347H | 85532 | 3349 |
| A573348H | 85533 | 3350 |
| A573349H | 85533 | 3351 |
| A573350H | 85534 | 3352 |
| A573351H | 85535 | 3353 |
| A573352H | 85536 | 3354 |
| A573353H | 85536 | 3355 |
| A573354H | 85537 | 3356 |
| A573355H | 85537 | 3357 |
| A573356H | 85538 | 3358 |
| A573357H | 85538 | 3359 |
| A573358H | 85539 | 3360 |
| A573359H | 85539 | 3361 |
| A573360H | 85539 | 3362 |
| A573361H | 85540 | 3363 |
| A573362H | 85540 | 3364 |
| A573363H | 85540 | 3365 |
| A573364H | 85541 | 3366 |
| A573365H | 85541 | 3367 |
| A573366H | 85541 | 3368 |
| A573367H | 85541 | 3369 |
| A573368H | 85542 | 3370 |
| A573369H | 85561 | 3371 |
| A573370H | 85561 | 3372 |
| A573371H | 85562 | 3373 |
| A573372H | 85562 | 3374 |
| A573373H | 85563 | 3375 |
| A573374H | 85563 | 3376 |
| A573375H | 85564 | 3377 |
| A573376H | 85565 | 3378 |
| A573377H | 85567 | 3379 |
| A573378H | 85569 | 3380 |
| A573379H | 85570 | 3381 |
| A573380H | 85572 | 3382 |
| A573381H | 85573 | 3383 |
| A573382H | 85575 | 3384 |
| A573383H | 85576 | 3385 |
| A573384H | 85576 | 3386 |
| A573385H | 85577 | 3387 |
| A573386H | 85579 | 3388 |
| A573387H | 85581 | 3389 |
| A573388H | 85582 | 3390 |
| A573389H | 85585 | 3391 |
| A573390H | 85586 | 3392 |
| A573391H | 85586 | 3393 |
| A573392H | 85587 | 3394 |
| A573393H | 85587 | 3395 |
| A573394H | 85588 | 3396 |
| A573395H | 85589 | 3397 |
| A573396H | 85592 | 3398 |
| A573397H | 85593 | 3399 |
| A573398H | 85597 | 3400 |
| A573399H | 85598 | 3401 |
| A573400H | 85598 | 3402 |
| A573401H | 85600 | 3403 |
| A573402H | 85601 | 3404 |
| A573403H | 85602 | 3405 |
| A573404H | 85603 | 3406 |
| A573405H | 85603 | 3407 |
| A573406H | 85604 | 3408 |
| A573407H | 85605 | 3409 |
| A573408H | 85606 | 3410 |
| A573409H | 85607 | 3411 |
| A573410H | 85607 | 3412 |
| A573411H | 85608 | 3413 |
| A573412H | 85610 | 3414 |
| A573413H | 85611 | 3415 |
| A573414H | 85612 | 3416 |
| A573415H | 85613 | 3417 |
| A573416H | 85614 | 3418 |
| A573417H | 85614 | 3419 |
| A573418H | 85634 | 3420 |
| A573419H | 85634 | 3421 |
| A573420Hi | 85658 | 3422 |
| A573421Hi | 85720 | 3423 |
| A573422Hi | 85721 | 3424 |
| A573423Hi | 85992 | 3425 |
| 86001-86500 | | |
| A57166Hi | 86182 | 168 |
| A57167Hi | 86462 | 169 |
| A573424Hi | 86179 | 3426 |
| A573425Hi | 86183 | 3427 |
| A573426Hi | 86184 | 3428 |
| A573427Hi | 86223 | 3429 |
| A573428Hi | 86223 | 3430 |
| A573429Hi | 86405 | 3431 |
| A573430Hi | 86406 | 3432 |
| A573431Hi | 86407 | 3433 |
| A573432Hi | 86407 | 3434 |
| A573433Hi | 86408 | 3435 |
| A573434Hi | 86410 | 3436 |
| A573435Hi | 86435 | 3437 |
| A573436Hi | 86461 | 3438 |
| 86501-87000 | | |
| A57168Hi | 86707 | 170 |
| A573437Hi | 86525 | 3439 |
| A573438Hi | 86629 | 3440 |
| A573439Hi | 86679 | 3441 |
| A573440Hi | 86708 | 3442 |
| A573441Hi | 86878 | 3443 |
| A573442Hi | 86878 | 3444 |
| A573443Hi | 86879 | 3445 |
| 87001-87500 | | |
| A57169Hi | 87122 | 171 |
| A57170Hi | 87444 | 172 |
| A573444Hi | 87110 | 3446 |
| A573445Hi | 87111 | 3447 |
| A573446Hi | 87112 | 3448 |
| A573447Hi | 87113 | 3449 |
| A573448Hi | 87113 | 3450 |
| A573449Hi | 87114 | 3451 |
| A573450Hi | 87115 | 3452 |
| A573451Hi | 87123 | 3453 |
| A573452Hi | 87154 | 3454 |
| A573453Hi | 87180 | 3455 |
| A573454Hi | 87181 | 3456 |
| A573455Hi | 87182 | 3457 |
| A573456Hi | 87183 | 3458 |
| A573457Hi | 87312 | 3459 |
| A573458Hi | 87320 | 3460 |
| A573459Hi | 87330 | 3461 |
| A573460Hi | 87331 | 3462 |
| A573461Hi | 87332 | 3463 |
| A573462Hi | 87333 | 3464 |
| A573463Hi | 87358 | 3465 |
| A573464Hi | 87440 | 3466 |
| A573465Hi | 87443 | 3467 |
| A573466Hi | 87454 | 3468 |
| A573467Hi | 87456 | 3469 |
| 87501-88000 | | |
| A57171Hi | 87994 | 173 |
| A57172Hi | 87997 | 174 |
| A573468Hi | 87534 | 3470 |
| A573469Hi | 87556 | 3471 |
| A573470Hi | 87578 | 3472 |
| A573471Hi | 87708 | 3473 |
| A573472Hi | 87957 | 3474 |
| A573473Hi | 87961 | 3475 |
| A573474Hi | 87997 | 3476 |
| A573475Hi | 87998 | 3477 |
| 88001-88500 | | |
| A573476Hi | 88139 | 3478 |
| A573477Hi | 88171 | 3479 |
| A573478Hi | 88172 | 3480 |
| A573479Hi | 88366 | 3481 |
| A573480Hi | 88367 | 3482 |
| A573481Hi | 88370 | 3483 |
| A573482Hi | 88370 | 3484 |
| A573483Hi | 88371 | 3485 |
| A573484Hi | 88372 | 3486 |
| A573485Hi | 88372 | 3487 |
| 88501-89000 | | |
| A57173Hi | 88896 | 175 |
| A57174Hi | 88905 | 176 |
| A573486Hi | 88584 | 3488 |
| A573487Hi | 88585 | 3489 |
| A573488Hi | 88586 | 3490 |
| A573489Hi | 88587 | 3491 |
| A573490Hi | 88593 | 3492 |
| A573491Hi | 88617 | 3493 |
| A573492Hi | 88617 | 3494 |
| A573493Hi | 88619 | 3495 |
| A573494Hi | 88622 | 3496 |
| A573495Hi | 88625 | 3497 |
| A573496Hi | 88627 | 3498 |
| A573497Hi | 88628 | 3499 |
| A573498Hi | 88893 | 3500 |
| A573499Hi | 88897 | 3501 |
| A573500Hi | 88897 | 3502 |
| A573501Hi | 88898 | 3503 |
| A573502Hi | 88900 | 3504 |
| A573503Hi | 88900 | 3505 |
| A573504Hi | 88902 | 3506 |
| A573505Hi | 88947 | 3507 |
| A573506Hi | 88947 | 3508 |
| A573507Hi | 88948 | 3509 |
| A573508Hi | 88948 | 3510 |
| A573509Hi | 88949 | 3511 |
| A573510Hi | 88950 | 3512 |
| A573511Hi | 88951 | 3513 |
| A573512Hi | 88951 | 3514 |
| A573513Hi | 88952 | 3515 |
| A573514H | 88991 | 3516 |
| A573515H | 88992 | 3517 |
| A573516H | 88993 | 3518 |
| A573517H | 88993 | 3519 |
| A573518H | 88997 | 3520 |
| A573519H | 88998 | 3521 |
| A573520H | 88999 | 3522 |
| A573521H | 88999 | 3523 |
| A573522H | 89000 | 3524 |
| A573523H | 89000 | 3525 |
| 89001-89500 | | |
| A57175Hi | 89376 | 177 |
| A573524H | 89001 | 3526 |
| A573525H | 89003 | 3527 |
| A573526H | 89004 | 3528 |
| A573527H | 89009 | 3529 |
| A573528H | 89011 | 3530 |
| A573529H | 89012 | 3531 |
| A573530H | 89013 | 3532 |
| A573531H | 89034 | 3533 |
| A573532H | 89035 | 3534 |
| A573533H | 89035 | 3535 |
| A573534H | 89036 | 3536 |
| A573535H | 89036 | 3537 |
| A573536H | 89036 | 3538 |
| A573537H | 89037 | 3539 |
| A573538H | 89037 | 3540 |
| A573539H | 89037 | 3541 |
| A573540H | 89038 | 3542 |
| A573541H | 89038 | 3543 |
| A573542H | 89038 | 3544 |
| A573543H | 89040 | 3545 |
| A573544H | 89041 | 3546 |
| A573545H | 89041 | 3547 |
| A573546H | 89042 | 3548 |
| A573547H | 89042 | 3549 |
| A573548H | 89042 | 3550 |
| A573549H | 89043 | 3551 |
| A573550H | 89043 | 3552 |
| A573551H | 89043 | 3553 |
| A573552H | 89043 | 3554 |
| A573553H | 89044 | 3555 |
| A573554H | 89044 | 3556 |
| A573555H | 89044 | 3557 |
| A573556H | 89044 | 3558 |
| A573557H | 89045 | 3559 |
| A573558H | 89045 | 3560 |
| A573559H | 89045 | 3561 |
| A573560H | 89046 | 3562 |
| A573561H | 89046 | 3563 |
| A573562H | 89047 | 3564 |
| A573563H | 89047 | 3565 |
| A573564H | 89047 | 3566 |
| A573565H | 89048 | 3567 |
| A573566H | 89049 | 3568 |
| A573567H | 89049 | 3569 |
| A573568H | 89050 | 3570 |
| A573569H | 89050 | 3571 |
| A573570H | 89050 | 3572 |
| A573571H | 89051 | 3573 |
| A573572H | 89058 | 3574 |
| A573573H | 89089 | 3575 |
| A573574H | 89090 | 3576 |
| A573575H | 89091 | 3577 |
| A573576H | 89092 | 3578 |
| A573577H | 89092 | 3579 |
| A573578H | 89093 | 3580 |
| A573579H | 89093 | 3581 |
| A573580H | 89094 | 3582 |
| A573581H | 89097 | 3583 |
| A573582H | 89098 | 3584 |
| A573583H | 89100 | 3585 |
| A573584H | 89125 | 3586 |
| A573585H | 89126 | 3587 |
| A573586H | 89132 | 3588 |
| A573587H | 89134 | 3589 |
| A573588H | 89135 | 3590 |
| A573589H | 89136 | 3591 |
| A573590H | 89137 | 3592 |
| A573591H | 89138 | 3593 |
| A573592H | 89138 | 3594 |
| A573593H | 89139 | 3595 |
| A573594H | 89140 | 3596 |
| A573595H | 89141 | 3597 |
| A573596H | 89141 | 3598 |
| A573597H | 89142 | 3599 |
| A573598H | 89143 | 3600 |
| A573599H | 89145 | 3601 |
| A573600H | 89150 | 3602 |
| A573601H | 89152 | 3603 |
| A573602H | 89153 | 3604 |
| A573603Hi | 89378 | 3605 |
| A573604Hi | 89379 | 3606 |
| A573605Hi | 89458 | 3607 |
| 89501-90000 | | |
| A57176Hi | 89684 | 178 |
| A57177Hi | 89742 | 179 |
| A57178Hi | 89817 | 180 |
| A573606Hi | 89621 | 3608 |
| A573607Hi | 89622 | 3609 |
| A573608Hi | 89623 | 3610 |
| A573609Hi | 89623 | 3611 |
| A573610Hi | 89624 | 3612 |
| A573611Hi | 89682 | 3613 |
| A573612Hi | 89682 | 3614 |
| A573613Hi | 89690 | 3615 |
| A573614Hi | 89691 | 3616 |
| A573615Hi | 89692 | 3617 |
| A573616Hi | 89692 | 3618 |
| A573617Hi | 89694 | 3619 |
| A573618Hi | 89699 | 3620 |
| A573619Hi | 89729 | 3621 |
| A573620Hi | 89731 | 3622 |
| A573621Hi | 89736 | 3623 |
| A573622Hi | 89739 | 3624 |
| A573623Hi | 89740 | 3625 |
| A573624Hi | 89748 | 3626 |
| A573625Hi | 89816 | 3627 |
| A573626Hi | 89866 | 3628 |
| 90001-90500 | | |
| A57179Hi | 90335 | 181 |
| A57180Hi | 90437 | 182 |
| A57181Hi | 90482 | 183 |
| A573627Hi | 90032 | 3629 |
| A573628Hi | 90033 | 3630 |
| A573629Hi | 90034 | 3631 |
| A573630Hi | 90059 | 3632 |
| A573631Hi | 90059 | 3633 |
| A573632Hi | 90064 | 3634 |
| A573633Hi | 90067 | 3635 |
| A573634Hi | 90182 | 3636 |
| A573635H | 90223 | 3637 |
| A573636H | 90227 | 3638 |
| A573637H | 90228 | 3639 |
| A573638H | 90229 | 3640 |
| A573639H | 90230 | 3641 |
| A573640H | 90232 | 3642 |
| A573641H | 90240 | 3643 |
| A573642H | 90242 | 3644 |
| A573643H | 90266 | 3645 |
| A573644H | 90267 | 3646 |
| A573645H | 90267 | 3647 |
| A573646H | 90269 | 3648 |
| A573647H | 90270 | 3649 |
| A573648H | 90273 | 3650 |
| A573649H | 90281 | 3651 |
| A573650H | 90282 | 3652 |
| A573651Hi | 90341 | 3653 |
| A573652Hi | 90342 | 3654 |
| A573653Hi | 90343 | 3655 |
| A573654Hi | 90393 | 3656 |
| A573655Hi | 90429 | 3657 |
| A573656Hi | 90431 | 3658 |
| A573657Hi | 90438 | 3659 |
| A573658Hi | 90466 | 3660 |
| A573659Hi | 90488 | 3661 |
| A573660Hi | 90491 | 3662 |
| A573661Hi | 90492 | 3663 |
| 90501-91000 | | |
| A57182Hi | 90672 | 184 |
| A573662Hi | 90521 | 3664 |
| A573663Hi | 90550 | 3665 |
| A573664Hi | 90553 | 3666 |
| A573665Hi | 90557 | 3667 |
| A573666Hi | 90558 | 3668 |
| A573667Hi | 90561 | 3669 |
| A573668Hi | 90563 | 3670 |
| A573669Hi | 90660 | 3671 |
| A573670Hi | 90662 | 3672 |
| A573671Hi | 90667 | 3673 |
| A573672Hi | 90670 | 3674 |
| A573673Hi | 90671 | 3675 |
| A573674Hi | 90671 | 3676 |
| A573675Hi | 90672 | 3677 |
| A573676Hi | 90673 | 3678 |
| A573677Hi | 90677 | 3679 |
| A573678Hi | 90678 | 3680 |
| A573679Hi | 90678 | 3681 |
| A573680Hi | 90679 | 3682 |
| A573681Hi | 90679 | 3683 |
| A573682Hi | 90869 | 3684 |
| A573683Hi | 90958 | 3685 |
| A573684Hi | 90959 | 3686 |
| 91001-91500 | | |
| A573685Hi | 91070 | 3687 |
| A573686Hi | 91206 | 3688 |
| A573687Hi | 91207 | 3689 |
| A573688Hi | 91209 | 3690 |
| A573689Hi | 91216 | 3691 |
| A573690Hi | 91227 | 3692 |
| A573691Hi | 91261 | 3693 |
| 91501-92000 | | |
| A57183Hi | 91810 | 185 |
| A573692Hi | 91570 | 3694 |
| A573693Hi | 91574 | 3695 |
| A573694Hi | 91811 | 3696 |
| A573695Hi | 91817 | 3697 |
| A573696Hi | 91817 | 3698 |
| A573697Hi | 91820 | 3699 |
| A573698Hi | 91820 | 3700 |
| A573699Hi | 91821 | 3701 |
| A573700Hi | 91821 | 3702 |
| A573701Hi | 91827 | 3703 |
| A573702Hi | 91829 | 3704 |
| A573703Hi | 91902 | 3705 |
| 92001-92500 | | |
| A57184Hi | 92086 | 186 |
| A573704Hi | 92034 | 3706 |
| A573705Hi | 92038 | 3707 |
| A573706Hi | 92084 | 3708 |
| A573707Hi | 92085 | 3709 |
| A573708Hi | 92085 | 3710 |
| A573709Hi | 92086 | 3711 |
| A573710Hi | 92087 | 3712 |
| A573711Hi | 92121 | 3713 |
| A573712Hi | 92121 | 3714 |
| A573713Hi | 92197 | 3715 |
| A573714Hi | 92200 | 3716 |
| A573715Hi | 92208 | 3717 |
| A573716Hi | 92209 | 3718 |
| A573717Hi | 92376 | 3719 |
| A573718Hi | 92377 | 3720 |
| A573719Hi | 92380 | 3721 |
| A573720Hi | 92382 | 3722 |
| A573721Hi | 92389 | 3723 |
| A573722Hi | 92390 | 3724 |
| A573723Hi | 92391 | 3725 |
| A573724Hi | 92391 | 3726 |
| A573725Hi | 92430 | 3727 |
| A573726Hi | 92432 | 3728 |
| A573727Hi | 92434 | 3729 |
| A573728Hi | 92436 | 3730 |
| A573729Hi | 92437 | 3731 |
| A573730Hi | 92438 | 3732 |
| A573731Hi | 92465 | 3733 |
| A573732Hi | 92466 | 3734 |
| A573733Hi | 92471 | 3735 |
| A573734Hi | 92472 | 3736 |
| A573735Hi | 92474 | 3737 |
| A573736Hi | 92475 | 3738 |
| A573737Hi | 92476 | 3739 |
| 92501-93000 | | |
| A57185Hi | 92545 | 187 |
| A57186Hi | 92574 | 188 |
| A573738Hi | 92547 | 3740 |
| A573739Hi | 92572 | 3741 |
| A573740Hi | 92582 | 3742 |
| A573741Hi | 92624 | 3743 |
| A573742Hi | 92711 | 3744 |
| A573743Hi | 92713 | 3745 |
| A573744Hi | 92720 | 3746 |
| A573745Hi | 92732 | 3747 |
| A573746Hi | 92733 | 3748 |
| A573747Hi | 92734 | 3749 |
| A573748Hi | 92780 | 3750 |
| A573749Hi | 92786 | 3751 |
| A573750Hi | 92791 | 3752 |
| A573751Hi | 92795 | 3753 |
| A573752Hi | 92797 | 3754 |
| A573753Hi | 92859 | 3755 |
| A573754Hi | 92909 | 3756 |
| A573755Hi | 92912 | 3757 |
| A573756Hi | 92978 | 3758 |
| 93001-93500 | | |
| A573757Hi | 93011 | 3759 |
| A573758Hi | 93011 | 3760 |
| A573759Hi | 93011 | 3761 |
| A573760Hi | 93012 | 3762 |
| A573761Hi | 93012 | 3763 |
| A573762Hi | 93094 | 3764 |
| A573763Hi | 93103 | 3765 |
| A573764Hi | 93104 | 3766 |
| A573765Hi | 93105 | 3767 |
| A573766Hi | 93108 | 3768 |
| A573767Hi | 93282 | 3769 |
| A573768Hi | 93290 | 3770 |
| A573769Hi | 93399 | 3771 |
| A573770Hi | 93400 | 3772 |
| A573771Hi | 93407 | 3773 |
| A573772Hi | 93407 | 3774 |
| 93501-94000 | | |
| A57187Hi | 93827 | 189 |
| A57246Hi | 93533 | 248 |
| A573773Hi | 93531 | 3775 |
| A573774Hi | 93562 | 3776 |
| A573775Hi | 93563 | 3777 |
| A573776Hi | 93570 | 3778 |
| A573777Hi | 93754 | 3779 |
| A573778Hi | 93826 | 3780 |
| A573779Hi | 93827 | 3781 |
| A573780Hi | 93828 | 3782 |
| A573781Hi | 93903 | 3783 |
| 94001-94500 | | |
| A573782Hi | 94004 | 3784 |
| A573783Hi | 94005 | 3785 |
| A573784Hi | 94014 | 3786 |
| A573785Hi | 94017 | 3787 |
| A573786Hi | 94478 | 3788 |
| A573787Hi | 94482 | 3789 |
| A573788Hi | 94484 | 3790 |
| A573789Hi | 94484 | 3791 |
| A573790Hi | 94485 | 3792 |
| A573791Hi | 94485 | 3793 |
| 94501-95000 | | |
| A573792Hi | 94511 | 3794 |
| A573793Hi | 94514 | 3795 |
| A573794Hi | 94516 | 3796 |
| A573795Hi | 94519 | 3797 |
| A573796Hi | 94520 | 3798 |
| A573797Hi | 94571 | 3799 |
| A573798Hi | 94572 | 3800 |
| A573799Hi | 94600 | 3801 |
| A573800Hi | 94654 | 3802 |
| A573801Hi | 94655 | 3803 |
| A573802Hi | 94656 | 3804 |
| A573803Hi | 94902 | 3805 |
| A573804Hi | 94903 | 3806 |
| A573805Hi | 94925 | 3807 |
| A573806H | 94992 | 3808 |
| A573807H | 94995 | 3809 |
| A573808H | 94999 | 3810 |
| 95001-95500 | | |
| A573809H | 95001 | 3811 |
| A573810H | 95002 | 3812 |
| A573811H | 95004 | 3813 |
| A573812H | 95005 | 3814 |
| A573813H | 95005 | 3815 |
| A573814H | 95006 | 3816 |
| A573815H | 95007 | 3817 |
| A573816H | 95008 | 3818 |
| A573817H | 95009 | 3819 |
| A573818H | 95009 | 3820 |
| A573819H | 95012 | 3821 |
| A573820H | 95013 | 3822 |
| A573821H | 95013 | 3823 |
| A573822H | 95015 | 3824 |
| A573823H | 95016 | 3825 |
| A573824H | 95017 | 3826 |
| A573825H | 95047 | 3827 |
| A573826H | 95088 | 3828 |
| A573827H | 95093 | 3829 |
| A573828H | 95099 | 3830 |
| A573829H | 95102 | 3831 |
| A573830H | 95106 | 3832 |
| A573831H | 95107 | 3833 |
| A573832H | 95113 | 3834 |
| A573833H | 95117 | 3835 |
| A573834H | 95118 | 3836 |
| A573835H | 95119 | 3837 |
| A573836H | 95120 | 3838 |
| A573837H | 95121 | 3839 |
| A573838H | 95129 | 3840 |
| A573839H | 95132 | 3841 |
| A573840H | 95133 | 3842 |
| A573841H | 95134 | 3843 |
| A573842H | 95135 | 3844 |
| A573843H | 95136 | 3845 |
| A573844H | 95138 | 3846 |
| A573845H | 95140 | 3847 |
| A573846H | 95142 | 3848 |
| A573847H | 95144 | 3849 |
| A573848H | 95156 | 3850 |
| A573849H | 95157 | 3851 |
| A573850H | 95158 | 3852 |
| A573851H | 95161 | 3853 |
| A573852Hi | 95207 | 3854 |
| A573853Hi | 95247 | 3855 |
| A573854Hi | 95248 | 3856 |
| A573855Hi | 95340 | 3857 |
| A573856Hi | 95489 | 3858 |
| A573857Hi | 95491 | 3859 |
| A573858Hi | 95492 | 3860 |
| 95501-96000 | | |
| A57188Hi | 95512 | 190 |
| A573859Hi | 95657 | 3861 |
| A573860Hi | 95683 | 3862 |
| A573861Hi | 95827 | 3863 |
| A573862Hi | 95829 | 3864 |
| A573863Hi | 95830 | 3865 |
| A573864Hi | 95831 | 3866 |
| A573865Hi | 95832 | 3867 |
| A573866Hi | 95832 | 3868 |
| A573867Hi | 95833 | 3869 |
| A573868Hi | 95835 | 3870 |
| A573869Hi | 95835 | 3871 |
| A573870Hi | 95836 | 3872 |
| A573871Hi | 95836 | 3873 |
| A573872Hi | 95841 | 3874 |
| A573873Hi | 96000 | 3875 |
| 96001-96500 | | |
| A57189Hi | 96006 | 191 |
| A573874Hi | 96001 | 3876 |
| A573875Hi | 96004 | 3877 |
| A573876Hi | 96005 | 3878 |
| A573877Hi | 96009 | 3879 |
| A573878H | 96045 | 3880 |
| A573879H | 96052 | 3881 |
| A573880H | 96053 | 3882 |
| A573881H | 96055 | 3883 |
| A573882H | 96057 | 3884 |
| A573883H | 96061 | 3885 |
| A573884H | 96063 | 3886 |
| A573885H | 96064 | 3887 |
| A573886H | 96064 | 3888 |
| A573887H | 96069 | 3889 |
| A573888H | 96077 | 3890 |
| A573889H | 96078 | 3891 |
| A573890H | 96079 | 3892 |
| A573891H | 96082 | 3893 |
| A573892H | 96083 | 3894 |
| A573893H | 96083 | 3895 |
| A573894H | 96084 | 3896 |
| A573895H | 96084 | 3897 |
| A573896H | 96085 | 3898 |
| A573897H | 96085 | 3899 |
| A573898H | 96086 | 3900 |
| A573899H | 96087 | 3901 |
| A573900H | 96087 | 3902 |
| A573901H | 96088 | 3903 |
| A573902H | 96090 | 3904 |
| A573903H | 96091 | 3905 |
| A573904H | 96092 | 3906 |
| A573905H | 96092 | 3907 |
| A573906H | 96093 | 3908 |
| A573907H | 96094 | 3909 |
| A573908H | 96094 | 3910 |
| A573909H | 96095 | 3911 |
| A573910H | 96095 | 3912 |
| A573911H | 96095 | 3913 |
| A573912H | 96096 | 3914 |
| A573913H | 96096 | 3915 |
| A573914H | 96096 | 3916 |
| A573915H | 96097 | 3917 |
| A573916H | 96098 | 3918 |
| A573917H | 96098 | 3919 |
| A573918H | 96099 | 3920 |
| A573919H | 96099 | 3921 |
| A573920H | 96099 | 3922 |
| A573921H | 96144 | 3923 |
| A573922H | 96144 | 3924 |
| A573923H | 96145 | 3925 |
| A573924H | 96145 | 3926 |
| A573925H | 96146 | 3927 |
| A573926H | 96146 | 3928 |
| A573927H | 96147 | 3929 |
| A573928H | 96147 | 3930 |
| A573929H | 96147 | 3931 |
| A573930H | 96148 | 3932 |
| A573931H | 96148 | 3933 |
| A573932H | 96148 | 3934 |
| A573933H | 96149 | 3935 |
| A573934H | 96150 | 3936 |
| A573935H | 96150 | 3937 |
| A573936H | 96151 | 3938 |
| A573937H | 96152 | 3939 |
| A573938H | 96152 | 3940 |
| A573939H | 96153 | 3941 |
| A573940H | 96153 | 3942 |
| A573941H | 96153 | 3943 |
| A573942H | 96156 | 3944 |
| A573943H | 96157 | 3945 |
| A573944H | 96158 | 3946 |
| A573945H | 96158 | 3947 |
| A573946H | 96159 | 3948 |
| A573947H | 96162 | 3949 |
| A573948H | 96164 | 3950 |
| A573949H | 96165 | 3951 |
| A573950H | 96166 | 3952 |
| A573951H | 96169 | 3953 |
| A573952H | 96170 | 3954 |
| A573953H | 96171 | 3955 |
| A573954H | 96171 | 3956 |
| A573955H | 96173 | 3957 |
| A573956Hi | 96317 | 3958 |
| A573957Hi | 96321 | 3959 |
| A573958Hi | 96383 | 3960 |
| A573959Hi | 96383 | 3961 |
| A573960Hi | 96483 | 3962 |
| 96501-97000 | | |
| A57190Hi | 96949 | 192 |
| A57247Hi | 96890 | 249 |
| A573961Hi | 96518 | 3963 |
| A573962Hi | 96519 | 3964 |
| A573963Hi | 96615 | 3965 |
| A573964Hi | 96663 | 3966 |
| A573965Hi | 96668 | 3967 |
| A573966Hi | 96672 | 3968 |
| A573967Hi | 96800 | 3969 |
| A573968Hi | 96801 | 3970 |
| A573969Hi | 96805 | 3971 |
| A573970Hi | 96806 | 3972 |
| A573971Hi | 96807 | 3973 |
| A573972Hi | 96807 | 3974 |
| A573973Hi | 96808 | 3975 |
| A573974Hi | 96814 | 3976 |
| A573975Hi | 96815 | 3977 |
| A573976Hi | 96815 | 3978 |
| A573977Hi | 96818 | 3979 |
| A573978Hi | 96886 | 3980 |
| A573979Hi | 96889 | 3981 |
| A573980Hi | 96894 | 3982 |
| A573981Hi | 96895 | 3983 |
| A573982Hi | 96895 | 3984 |
| 97001-97500 | | |
| A57191Hi | 97276 | 193 |
| A57192Hi | 97277 | 194 |
| A573983H | 97039 | 3985 |
| A573984H | 97039 | 3986 |
| A573985H | 97039 | 3987 |
| A573986H | 97040 | 3988 |
| A573987H | 97040 | 3989 |
| A573988H | 97040 | 3990 |
| A573989H | 97041 | 3991 |
| A573990H | 97041 | 3992 |
| A573991H | 97041 | 3993 |
| A573992H | 97042 | 3994 |
| A573993H | 97042 | 3995 |
| A573994H | 97043 | 3996 |
| A573995H | 97043 | 3997 |
| A573996H | 97043 | 3998 |
| A573997H | 97044 | 3999 |
| A573998H | 97044 | 4000 |
| A573999H | 97045 | 4001 |
| A574000H | 97045 | 4002 |
| A574001H | 97046 | 4003 |
| A574002H | 97047 | 4004 |
| A574003H | 97051 | 4005 |
| A574004H | 97054 | 4006 |
| A574005H | 97055 | 4007 |
| A574006H | 97055 | 4008 |
| A574007H | 97056 | 4009 |
| A574008H | 97056 | 4010 |
| A574009H | 97057 | 4011 |
| A574010H | 97057 | 4012 |
| A574011H | 97057 | 4013 |
| A574012H | 97058 | 4014 |
| A574013H | 97058 | 4015 |
| A574014H | 97058 | 4016 |
| A574015H | 97059 | 4017 |
| A574016H | 97059 | 4018 |
| A574017H | 97059 | 4019 |
| A574018H | 97060 | 4020 |
| A574019H | 97060 | 4021 |
| A574020H | 97060 | 4022 |
| A574021H | 97061 | 4023 |
| A574022H | 97061 | 4024 |
| A574023H | 97061 | 4025 |
| A574024H | 97062 | 4026 |
| A574025H | 97062 | 4027 |
| A574026H | 97062 | 4028 |
| A574027H | 97084 | 4029 |
| A574028H | 97086 | 4030 |
| A574029H | 97087 | 4031 |
| A574030H | 97088 | 4032 |
| A574031H | 97088 | 4033 |
| A574032H | 97089 | 4034 |
| A574033H | 97089 | 4035 |
| A574034H | 97089 | 4036 |
| A574035H | 97095 | 4037 |
| A574036H | 97096 | 4038 |
| A574037H | 97096 | 4039 |
| A574038H | 97097 | 4040 |
| A574039H | 97098 | 4041 |
| A574040H | 97098 | 4042 |
| A574041H | 97099 | 4043 |
| A574042H | 97099 | 4044 |
| A574043H | 97104 | 4045 |
| A574044H | 97107 | 4046 |
| A574045H | 97112 | 4047 |
| A574046H | 97114 | 4048 |
| A574047H | 97116 | 4049 |
| A574048H | 97118 | 4050 |
| A574049H | 97119 | 4051 |
| A574050H | 97120 | 4052 |
| A574051H | 97120 | 4053 |
| A574052H | 97123 | 4054 |
| A574053H | 97161 | 4055 |
| A574054H | 97163 | 4056 |
| A574055H | 97324 | 4057 |
| A574056H | 97325 | 4058 |
| A574057H | 97326 | 4059 |
| A574058H | 97326 | 4060 |
| A574059H | 97327 | 4061 |
| A574060H | 97328 | 4062 |
| A574061H | 97329 | 4063 |
| A574062H | 97333 | 4064 |
| A574063H | 97336 | 4065 |
| A574064H | 97340 | 4066 |
| A574065H | 97342 | 4067 |
| A574066H | 97343 | 4068 |
| A574067H | 97343 | 4069 |
| A574068H | 97347 | 4070 |
| A574069H | 97349 | 4071 |
| A574070H | 97377 | 4072 |
| A574071H | 97378 | 4073 |
| A574072H | 97378 | 4074 |
| A574073H | 97380 | 4075 |
| A574074H | 97384 | 4076 |
| A574075H | 97385 | 4077 |
| A574076H | 97386 | 4078 |
| A574077H | 97386 | 4079 |
| A574078H | 97395 | 4080 |
| A574079H | 97396 | 4081 |
| A574080H | 97396 | 4082 |
| A574081H | 97400 | 4083 |
| A574082H | 97402 | 4084 |
| A574083H | 97403 | 4085 |
| A574084H | 97403 | 4086 |
| A574085H | 97409 | 4087 |
| A574086H | 97410 | 4088 |
| A574087H | 97411 | 4089 |
| A574088H | 97412 | 4090 |
| A574089H | 97420 | 4091 |
| A574090H | 97426 | 4092 |
| A574091H | 97429 | 4093 |
| A574092H | 97448 | 4094 |
| A574093H | 97471 | 4095 |
| A574094H | 97472 | 4096 |
| A574095H | 97478 | 4097 |
| A574096H | 97479 | 4098 |
| A574097H | 97480 | 4099 |
| A574098H | 97480 | 4100 |
| A574099H | 97481 | 4101 |
| A574100H | 97488 | 4102 |
| 97501-98000 | | |
| A574101Hi | 97682 | 4103 |
| A574102Hi | 97842 | 4104 |
| A574103Hi | 97864 | 4105 |
| A574104Hi | 97896 | 4106 |
| A574105Hi | 97898 | 4107 |
| A574106Hi | 97905 | 4108 |
| A574107Hi | 97908 | 4109 |
| A574108Hi | 97909 | 4110 |
| A574109Hi | 97915 | 4111 |
| A574110Hi | 97917 | 4112 |
| A574111Hi | 97918 | 4113 |
| A574112Hi | 97920 | 4114 |
| A574113Hi | 97920 | 4115 |
| A574114Hi | 97938 | 4116 |
| A574115Hi | 97940 | 4117 |
| A574116Hi | 97942 | 4118 |
| A574117Hi | 97942 | 4119 |
| A574118Hi | 97944 | 4120 |
| A574119Hi | 97945 | 4121 |
| 98001-98500 | | |
| A57193Hi | 98498 | 195 |
| A574120Hi | 98114 | 4122 |
| A574121H | 98164 | 4123 |
| A574122H | 98164 | 4124 |
| A574123H | 98173 | 4125 |
| A574124H | 98174 | 4126 |
| A574125H | 98175 | 4127 |
| A574126H | 98175 | 4128 |
| A574127H | 98177 | 4129 |
| A574128H | 98178 | 4130 |
| A574129H | 98179 | 4131 |
| A574130H | 98179 | 4132 |
| A574131H | 98180 | 4133 |
| A574132H | 98181 | 4134 |
| A574133H | 98181 | 4135 |
| A574134H | 98182 | 4136 |
| A574135H | 98183 | 4137 |
| A574136H | 98183 | 4138 |
| A574137H | 98184 | 4139 |
| A574138H | 98184 | 4140 |
| A574139H | 98186 | 4141 |
| A574140H | 98187 | 4142 |
| A574141H | 98187 | 4143 |
| A574142H | 98188 | 4144 |
| A574143H | 98188 | 4145 |
| A574144H | 98189 | 4146 |
| A574145H | 98192 | 4147 |
| A574146H | 98193 | 4148 |
| A574147H | 98193 | 4149 |
| A574148H | 98202 | 4150 |
| A574149H | 98204 | 4151 |
| A574150H | 98205 | 4152 |
| A574151H | 98205 | 4153 |
| A574152H | 98206 | 4154 |
| A574153H | 98206 | 4155 |
| A574154H | 98207 | 4156 |
| A574155H | 98207 | 4157 |
| A574156H | 98208 | 4158 |
| A574157H | 98208 | 4159 |
| A574158H | 98210 | 4160 |
| A574159H | 98213 | 4161 |
| A574160H | 98214 | 4162 |
| A574161H | 98215 | 4163 |
| A574162H | 98216 | 4164 |
| A574163H | 98216 | 4165 |
| A574164H | 98217 | 4166 |
| A574165H | 98217 | 4167 |
| A574166H | 98219 | 4168 |
| A574167H | 98220 | 4169 |
| A574168H | 98223 | 4170 |
| A574169H | 98225 | 4171 |
| A574170H | 98226 | 4172 |
| A574171H | 98227 | 4173 |
| A574172H | 98227 | 4174 |
| A574173H | 98228 | 4175 |
| A574174H | 98229 | 4176 |
| A574175H | 98230 | 4177 |
| A574176H | 98231 | 4178 |
| A574177H | 98231 | 4179 |
| A574178H | 98232 | 4180 |
| A574179H | 98233 | 4181 |
| A574180H | 98234 | 4182 |
| A574181H | 98234 | 4183 |
| A574182H | 98235 | 4184 |
| A574183H | 98236 | 4185 |
| A574184H | 98237 | 4186 |
| A574185H | 98239 | 4187 |
| A574186H | 98242 | 4188 |
| A574187H | 98246 | 4189 |
| A574188H | 98278 | 4190 |
| A574189H | 98283 | 4191 |
| A574190H | 98284 | 4192 |
| A574191H | 98285 | 4193 |
| A574192H | 98286 | 4194 |
| A574193H | 98286 | 4195 |
| A574194H | 98291 | 4196 |
| A574195H | 98292 | 4197 |
| A574196H | 98292 | 4198 |
| A574197Hi | 98355 | 4199 |
| A574198Hi | 98500 | 4200 |
| 98501-99000 | | |
| A57194Hi | 98522 | 196 |
| A574199Hi | 98501 | 4201 |
| A574200Hi | 98502 | 4202 |
| A574201Hi | 98509 | 4203 |
| A574202Hi | 98571 | 4204 |
| A574203Hi | 98571 | 4205 |
| A574204Hi | 98572 | 4206 |
| A574205Hi | 98626 | 4207 |
| A574206Hi | 98666 | 4208 |
| A574207Hi | 98666 | 4209 |
| A574208Hi | 98668 | 4210 |
| A574209Hi | 98669 | 4211 |
| A574210Hi | 98669 | 4212 |
| A574211Hi | 98670 | 4213 |
| A574212Hi | 98672 | 4214 |
| A574213Hi | 98673 | 4215 |
| A574214Hi | 98714 | 4216 |
| A574215Hi | 98721 | 4217 |
| A574216Hi | 98731 | 4218 |
| A574217Hi | 98889 | 4219 |
| A574218Hi | 98931 | 4220 |
| A574219Hi | 98968 | 4221 |
| 99001-99500 | | |
| A57195Hi | 99316 | 197 |
| A574220Hi | 99319 | 4222 |
| 99501-100000 | | |
| A57196Hi | 99612 | 198 |
| A57197Hi | 99754 | 199 |
| A57198Hi | 99941 | 200 |
| A57199Hi | 99968 | 201 |
| A57200Hi | 99969 | 202 |
| A57201Hi | 99969 | 203 |
| A57202Hi | 99980 | 204 |
| A574221Hi | 99613 | 4223 |
| A574222Hi | 99613 | 4224 |
| A574223Hi | 99833 | 4225 |
| A574224Hi | 99833 | 4226 |
| A574225Hi | 99841 | 4227 |
| A574226Hi | 99970 | 4228 |
| A574227Hi | 99971 | 4229 |
| A574228Hi | 99972 | 4230 |
| A574229Hi | 99972 | 4231 |
| A574230Hi | 99973 | 4232 |
| A574231Hi | 99974 | 4233 |
| A574232Hi | 99979 | 4234 |
| A574233Hi | 99981 | 4235 |
| A574234Hi | 99981 | 4236 |
| A574235Hi | 99984 | 4237 |
| A574236Hi | 99985 | 4238 |
| A574237Hi | 99985 | 4239 |
| A574238Hi | 99986 | 4240 |
| A574239Hi | 99994 | 4241 |
| A574240Hi | 99995 | 4242 |
| A574241Hi | 99996 | 4243 |
| 100001-100500 | | |
| A57203Hi | 100415 | 205 |
| A574242Hi | 100406 | 4244 |
| A574243Hi | 100408 | 4245 |
| A574244Hi | 100414 | 4246 |
| 100501-101000 | | |
| A57204Hi | 100594 | 206 |
| A57205Hi | 100809 | 207 |
| A574245Hi | 100590 | 4247 |
| A574246Hi | 100592 | 4248 |
| A574247Hi | 100592 | 4249 |
| A574248Hi | 100641 | 4250 |
| A574249Hi | 100680 | 4251 |
| A574250Hi | 100680 | 4252 |
| A574251Hi | 100681 | 4253 |
| A574252Hi | 100840 | 4254 |
| A574253Hi | 100842 | 4255 |
| A574254Hi | 100844 | 4256 |
| 101001-101500 | | |
| A574255Hi | 101017 | 4257 |
| A574256Hi | 101018 | 4258 |
| A574257Hi | 101103 | 4259 |
| A574258Hi | 101104 | 4260 |
| A574259Hi | 101105 | 4261 |
| A574260Hi | 101110 | 4262 |
| 101501-102000 | | |
| A574261Hi | 101643 | 4263 |
| A574262Hi | 101643 | 4264 |
| A574263Hi | 101765 | 4265 |
| A574264Hi | 101775 | 4266 |
| A574265Hi | 101824 | 4267 |
| A574266Hi | 101825 | 4268 |
| A574267Hi | 101837 | 4269 |
| A574268Hi | 101839 | 4270 |
| A574269Hi | 101844 | 4271 |
| 102001-102500 | | |
| A57206Hi | 102072 | 208 |
| A57207Hi | 102111 | 209 |
| A575329Hi | 102111 | 209 |
| A575330Hi | 102111 | 209 |
| A575332Hi | 102111 | 209 |
| A575335Hi | 102111 | 209 |
| A575336Hi | 102111 | 209 |
| A575337Hi | 102111 | 209 |
| A575338Hi | 102111 | 209 |
| A575340Hi | 102111 | 209 |
| A575341Hi | 102111 | 209 |
| A57208Hi | 102112 | 210 |
| A575343Hi | 102112 | 210 |
| A575344Hi | 102112 | 210 |
| A575346Hi | 102112 | 210 |
| A575349Hi | 102112 | 210 |
| A575350Hi | 102112 | 210 |
| A575351Hi | 102112 | 210 |
| A575352Hi | 102112 | 210 |
| A575354Hi | 102112 | 210 |
| A575355Hi | 102112 | 210 |
| A57209Hi | 102114 | 211 |
| A57210Hi | 102304 | 212 |
| A574270Hi | 102004 | 4272 |
| A574271Hi | 102007 | 4273 |
| A574272Hi | 102064 | 4274 |
| A574273Hi | 102069 | 4275 |
| A574274Hi | 102073 | 4276 |
| A574275Hi | 102081 | 4277 |
| A574276Hi | 102087 | 4278 |
| A574277Hi | 102088 | 4279 |
| A574278Hi | 102113 | 4280 |
| A574279Hi | 102126 | 4281 |
| A574280Hi | 102269 | 4282 |
| A574281Hi | 102270 | 4283 |
| A574282Hi | 102274 | 4284 |
| A574283Hi | 102304 | 4285 |
| A574284Hi | 102304 | 4286 |
| A574285Hi | 102306 | 4287 |
| 102501-103000 | | |
| A57211Hi | 102569 | 213 |
| A57212Hi | 102915 | 214 |
| A57213Hi | 102980 | 215 |
| A574286Hi | 102570 | 4288 |
| A574287Hi | 102573 | 4289 |
| A574288Hi | 102574 | 4290 |
| A574289Hi | 102643 | 4291 |
| A574290Hi | 102644 | 4292 |
| A574291Hi | 102648 | 4293 |
| A574292Hi | 102693 | 4294 |
| A574293Hi | 102697 | 4295 |
| A574294Hi | 102697 | 4296 |
| A574295Hi | 102760 | 4297 |
| A574296Hi | 102912 | 4298 |
| A574297Hi | 102920 | 4299 |
| A574298Hi | 102980 | 4300 |
| A574299Hi | 102984 | 4301 |
| 103001-103500 | | |
| A57214Hi | 103051 | 216 |
| A57215Hi | 103101 | 217 |
| A57216Hi | 103115 | 218 |
| A574300Hi | 103047 | 4302 |
| A574301Hi | 103054 | 4303 |
| A574302Hi | 103055 | 4304 |
| A574303Hi | 103055 | 4305 |
| A574304Hi | 103112 | 4306 |
| A574305Hi | 103113 | 4307 |
| A574306Hi | 103122 | 4308 |
| A574307Hi | 103218 | 4309 |
| A574308Hi | 103219 | 4310 |
| A574309Hi | 103226 | 4311 |
| A574310Hi | 103228 | 4312 |
| A574311Hi | 103229 | 4313 |
| A574312Hi | 103229 | 4314 |
| A574313Hi | 103332 | 4315 |
| A574314H | 103368 | 4316 |
| A574315H | 103369 | 4317 |
| A574316H | 103390 | 4318 |
| A574317H | 103390 | 4319 |
| A574318H | 103405 | 4320 |
| A574319H | 103410 | 4321 |
| 103501-104000 | | |
| A574320Hi | 103864 | 4322 |
| A574321Hi | 103895 | 4323 |
| A574322Hi | 103898 | 4324 |
| A574323Hi | 103908 | 4325 |
| 104001-104500 | | |
| A574324Hi | 104100 | 4326 |
| A574325Hi | 104161 | 4327 |
| A574326Hi | 104191 | 4328 |
| A574327Hi | 104327 | 4329 |
| A574328Hi | 104336 | 4330 |
| A574329Hi | 104346 | 4331 |
| 105001-105500 | | |
| A574330Hi | 105020 | 4332 |
| A574331Hi | 105034 | 4333 |
| A574332Hi | 105039 | 4334 |
| A574333Hi | 105040 | 4335 |
| A574334Hi | 105077 | 4336 |
| A574335Hi | 105078 | 4337 |
| 105501-106000 | | |
| A57217Hi | 105711 | 219 |
| 106001-106500 | | |
| A574336Hi | 106255 | 4338 |
| A574337Hi | 106256 | 4339 |
| A574338Hi | 106259 | 4340 |
| 106501-107000 | | |
| A574339Hi | 106746 | 4341 |
| 107001-107500 | | |
| A574340Hi | 107017 | 4342 |
| A574341Hi | 107025 | 4343 |
| A574342Hi | 107313 | 4344 |
| A574343Hi | 107464 | 4345 |
| A574344Hi | 107465 | 4346 |
| 108001-108500 | | |
| A574345Hi | 108224 | 4347 |
| A574346Hi | 108230 | 4348 |
| A574347Hi | 108360 | 4349 |
| A574348Hi | 108370 | 4350 |
| 108501-109000 | | |
| A574349Hi | 108700 | 4351 |
| 109001-109500 | | |
| A574350Hi | 109144 | 4352 |
| A574351Hi | 109147 | 4353 |
| A574352Hi | 109150 | 4354 |
| A574353Hi | 109154 | 4355 |
| A574354Hi | 109157 | 4356 |
| A574355Hi | 109161 | 4357 |
| 109501-110000 | | |
| A57218Hi | 109904 | 220 |
| A574356Hi | 109849 | 4358 |
| A574357H | 109977 | 4359 |
| A574358H | 109984 | 4360 |
| A574359H | 109992 | 4361 |
| A574360H | 109993 | 4362 |
| A574361H | 109994 | 4363 |
| A574362H | 109996 | 4364 |
| 110001-110500 | | |
| A574363H | 110004 | 4365 |
| A574364H | 110008 | 4366 |
| A574365H | 110009 | 4367 |
| A574366H | 110009 | 4368 |
| A574367H | 110010 | 4369 |
| A574368H | 110032 | 4370 |
| A574369H | 110033 | 4371 |
| A574370H | 110033 | 4372 |
| A574371H | 110034 | 4373 |
| A574372H | 110034 | 4374 |
| A574373H | 110034 | 4375 |
| A574374H | 110035 | 4376 |
| A574375H | 110039 | 4377 |
| A574376H | 110040 | 4378 |
| A574377H | 110040 | 4379 |
| A574378H | 110045 | 4380 |
| A574379H | 110047 | 4381 |
| A574380H | 110048 | 4382 |
| A574381H | 110049 | 4383 |
| A574382H | 110050 | 4384 |
| A574383H | 110052 | 4385 |
| A574384H | 110055 | 4386 |
| A574385H | 110056 | 4387 |
| A574386H | 110057 | 4388 |
| A574387H | 110057 | 4389 |
| A574388H | 110058 | 4390 |
| A574389H | 110058 | 4391 |
| A574390H | 110059 | 4392 |
| A574391H | 110059 | 4393 |
| A574392H | 110060 | 4394 |
| A574393H | 110061 | 4395 |
| A574394H | 110064 | 4396 |
| A574395H | 110065 | 4397 |
| A574396H | 110066 | 4398 |
| A574397H | 110066 | 4399 |
| A574398H | 110067 | 4400 |
| A574399H | 110067 | 4401 |
| A574400H | 110068 | 4402 |
| A574401H | 110069 | 4403 |
| A574402H | 110070 | 4404 |
| A574403H | 110070 | 4405 |
| A574404H | 110071 | 4406 |
| A574405H | 110071 | 4407 |
| A574406H | 110072 | 4408 |
| A574407H | 110072 | 4409 |
| A574408H | 110072 | 4410 |
| A574409H | 110073 | 4411 |
| A574410H | 110074 | 4412 |
| A574411H | 110074 | 4413 |
| A574412H | 110075 | 4414 |
| A574413H | 110075 | 4415 |
| A574414H | 110076 | 4416 |
| A574415H | 110076 | 4417 |
| A574416H | 110076 | 4418 |
| A574417H | 110077 | 4419 |
| A574418H | 110077 | 4420 |
| A574419H | 110079 | 4421 |
| A574420H | 110080 | 4422 |
| A574421H | 110081 | 4423 |
| A574422H | 110081 | 4424 |
| A574423H | 110082 | 4425 |
| A574424H | 110082 | 4426 |
| A574425H | 110083 | 4427 |
| A574426H | 110084 | 4428 |
| A574427H | 110084 | 4429 |
| A574428H | 110088 | 4430 |
| A574429H | 110089 | 4431 |
| A574430H | 110097 | 4432 |
| A574431H | 110098 | 4433 |
| A574432H | 110099 | 4434 |
| A574433H | 110100 | 4435 |
| A574434H | 110101 | 4436 |
| A574435H | 110102 | 4437 |
| A574436H | 110103 | 4438 |
| A574437H | 110103 | 4439 |
| A574438H | 110104 | 4440 |
| A574439H | 110104 | 4441 |
| A574440H | 110105 | 4442 |
| A574441H | 110109 | 4443 |
| A574442H | 110110 | 4444 |
| A574443H | 110110 | 4445 |
| A574444H | 110111 | 4446 |
| A574445H | 110114 | 4447 |
| A574446H | 110115 | 4448 |
| A574447H | 110115 | 4449 |
| A574448H | 110116 | 4450 |
| A574449H | 110116 | 4451 |
| A574450H | 110118 | 4452 |
| A574451H | 110119 | 4453 |
| A574452H | 110120 | 4454 |
| A574453H | 110121 | 4455 |
| A574454H | 110121 | 4456 |
| A574455H | 110122 | 4457 |
| A574456H | 110122 | 4458 |
| A574457H | 110124 | 4459 |
| A574458H | 110125 | 4460 |
| A574459H | 110125 | 4461 |
| A574460Hi | 110351 | 4462 |
| A574461Hi | 110416 | 4463 |
| A574462Hi | 110475 | 4464 |
| A574463Hi | 110497 | 4465 |
| 110501-111000 | | |
| A574464Hi | 110503 | 4466 |
| A574465Hi | 110504 | 4467 |
| A574466Hi | 110507 | 4468 |
| 111001-111500 | | |
| A57219Hi | 111091 | 221 |
| A574467Hi | 111419 | 4469 |
| A574468Hi | 111422 | 4470 |
| A574469Hi | 111423 | 4471 |
| 111501-112000 | | |
| A574470Hi | 111625 | 4472 |
| A574471Hi | 111838 | 4473 |
| A574472Hi | 111932 | 4474 |
| A574473Hi | 111933 | 4475 |
| 112001-112500 | | |
| A574474Hi | 112135 | 4476 |
| A574475Hi | 112136 | 4477 |
| A574476Hi | 112136 | 4478 |
| A574477Hi | 112137 | 4479 |
| A574478Hi | 112138 | 4480 |
| A574479Hi | 112139 | 4481 |
| A574480Hi | 112250 | 4482 |
| A574481Hi | 112251 | 4483 |
| A574482Hi | 112282 | 4484 |
| A574483Hi | 112448 | 4485 |
| A574484Hi | 112453 | 4486 |
| A574485Hi | 112454 | 4487 |
| A574486Hi | 112455 | 4488 |
| 112501-113000 | | |
| A574487Hi | 112993 | 4489 |
| 113001-113500 | | |
| A574488Hi | 113245 | 4490 |
| A574489Hi | 113295 | 4491 |
| A574490Hi | 113296 | 4492 |
| A574491Hi | 113316 | 4493 |
| A574492Hi | 113389 | 4494 |
| A574493Hi | 113394 | 4495 |
| 113501-114000 | | |
| A574494Hi | 113502 | 4496 |
| A574495Hi | 113619 | 4497 |
| A574496Hi | 113660 | 4498 |
| A574497Hi | 113722 | 4499 |
| A574498Hi | 113782 | 4500 |
| A574499Hi | 113927 | 4501 |
| 114001-114500 | | |
| A57220Hi | 114218 | 222 |
| A574500Hi | 114215 | 4502 |
| A574501Hi | 114216 | 4503 |
| A574502Hi | 114216 | 4504 |
| A574503Hi | 114217 | 4505 |
| A574504Hi | 114219 | 4506 |
| A574505Hi | 114321 | 4507 |
| A574506Hi | 114435 | 4508 |
| A574507Hi | 114436 | 4509 |
| 114501-115000 | | |
| A574508Hi | 114678 | 4510 |
| 115001-115500 | | |
| A574509H | 115308 | 4511 |
| A574510H | 115309 | 4512 |
| A574511H | 115309 | 4513 |
| A574512H | 115310 | 4514 |
| A574513H | 115310 | 4515 |
| A574514H | 115311 | 4516 |
| A574515H | 115311 | 4517 |
| A574516H | 115312 | 4518 |
| A574517H | 115313 | 4519 |
| A574518H | 115313 | 4520 |
| A574519H | 115339 | 4521 |
| A574520H | 115340 | 4522 |
| A574521H | 115342 | 4523 |
| A574522H | 115350 | 4524 |
| A574523H | 115353 | 4525 |
| A574524H | 115356 | 4526 |
| A574525H | 115357 | 4527 |
| A574526H | 115358 | 4528 |
| A574527H | 115358 | 4529 |
| A574528H | 115359 | 4530 |
| A574529H | 115359 | 4531 |
| A574530H | 115360 | 4532 |
| A574531H | 115361 | 4533 |
| A574532H | 115361 | 4534 |
| A574533H | 115362 | 4535 |
| A574534H | 115362 | 4536 |
| A574535H | 115363 | 4537 |
| A574536H | 115363 | 4538 |
| A574537H | 115364 | 4539 |
| A574538H | 115364 | 4540 |
| A574539H | 115364 | 4541 |
| A574540H | 115365 | 4542 |
| A574541H | 115366 | 4543 |
| A574542H | 115366 | 4544 |
| A574543H | 115367 | 4545 |
| A574544H | 115373 | 4546 |
| A574545H | 115374 | 4547 |
| A574546H | 115374 | 4548 |
| A574547H | 115377 | 4549 |
| A574548H | 115382 | 4550 |
| A574549H | 115385 | 4551 |
| A574550H | 115386 | 4552 |
| A574551H | 115387 | 4553 |
| A574552H | 115388 | 4554 |
| A574553H | 115388 | 4555 |
| A574554H | 115389 | 4556 |
| A574555H | 115390 | 4557 |
| A574556H | 115390 | 4558 |
| A574557H | 115393 | 4559 |
| A574558H | 115395 | 4560 |
| A574559H | 115396 | 4561 |
| A574560H | 115396 | 4562 |
| A574561H | 115397 | 4563 |
| A574562H | 115397 | 4564 |
| A574563H | 115397 | 4565 |
| A574564H | 115398 | 4566 |
| A574565H | 115398 | 4567 |
| A574566H | 115398 | 4568 |
| A574567H | 115401 | 4569 |
| A574568H | 115402 | 4570 |
| A574569H | 115402 | 4571 |
| A574570H | 115403 | 4572 |
| A574571H | 115403 | 4573 |
| A574572H | 115403 | 4574 |
| A574573H | 115404 | 4575 |
| A574574H | 115415 | 4576 |
| A574575H | 115416 | 4577 |
| A574576Hi | 115489 | 4578 |
| A574577Hi | 115496 | 4579 |
| A574578Hi | 115497 | 4580 |
| A574579Hi | 115499 | 4581 |
| 115501-116000 | | |
| A574580Hi | 115515 | 4582 |
| A574581Hi | 115797 | 4583 |
| 116001-116500 | | |
| A574582Hi | 116122 | 4584 |
| A574583Hi | 116278 | 4585 |
| A574584Hi | 116284 | 4586 |
| 116501-117000 | | |
| A574585Hi | 116513 | 4587 |
| A574586Hi | 116725 | 4588 |
| A574587Hi | 116773 | 4589 |
| 117001-117500 | | |
| A574588Hi | 117035 | 4590 |
| A574589Hi | 117046 | 4591 |
| A574590Hi | 117238 | 4592 |
| A574591Hi | 117323 | 4593 |
| 117501-118000 | | |
| A574592Hi | 117897 | 4594 |
| A574593Hi | 117900 | 4595 |
| 118001-118500 | | |
| A574594Hi | 118115 | 4596 |
| A574595Hi | 118227 | 4597 |
| A574596Hi | 118473 | 4598 |
| A574597Hi | 118474 | 4599 |
| A574598Hi | 118477 | 4600 |
| 118501-119000 | | |
| A574599Hi | 118849 | 4601 |
| A574600Hi | 118854 | 4602 |
| A574601Hi | 118855 | 4603 |
| 119001-119500 | | |
| A574602Hi | 119475 | 4604 |
| 119501-120000 | | |
| A57221Hi | 119686 | 223 |
| 120001-120500 | | |
| A574603Hi | 120017 | 4605 |
| A574604Hi | 120095 | 4606 |
| 120501-121000 | | |
| A57222Hi | 120997 | 224 |
| A574605Hi | 120966 | 4607 |
| A574606Hi | 120971 | 4608 |
| 121001-121500 | | |
| A574607Hi | 121272 | 4609 |
| A574608Hi | 121361 | 4610 |
| A574609Hi | 121461 | 4611 |
| 122001-122500 | | |
| A574610Hi | 122179 | 4612 |
| A574611Hi | 122235 | 4613 |
| 122501-123000 | | |
| A574612Hi | 122605 | 4614 |
| 123001-123500 | | |
| A574613Hi | 123023 | 4615 |
| A574614Hi | 123029 | 4616 |
| A574615Hi | 123036 | 4617 |
| A574616Hi | 123037 | 4618 |
| A574617Hi | 123230 | 4619 |
| 124001-124500 | | |
| A574618Hi | 124361 | 4620 |
| A574619Hi | 124363 | 4621 |
| 124501-125000 | | |
| A574620Hi | 124651 | 4622 |
| A574621Hi | 124652 | 4623 |
| 125001-125500 | | |
| A574622Hi | 125199 | 4624 |
| A574623Hi | 125261 | 4625 |
| A574624Hi | 125264 | 4626 |
| A574625Hi | 125302 | 4627 |
| A574626Hi | 125469 | 4628 |
| 125501-126000 | | |
| A57223Hi | 125919 | 225 |
| A574627Hi | 125502 | 4629 |
| A574628Hi | 125644 | 4630 |
| A574629Hi | 125924 | 4631 |
| 126001-126500 | | |
| A574630Hi | 126151 | 4632 |
| A574631Hi | 126155 | 4633 |
| A574632Hi | 126160 | 4634 |
| A574633Hi | 126163 | 4635 |
| A574634Hi | 126164 | 4636 |
| A574635Hi | 126164 | 4637 |
| A574636Hi | 126447 | 4638 |
| 127001-127500 | | |
| A574637Hi | 127160 | 4639 |
| A574638Hi | 127161 | 4640 |
| A574639Hi | 127162 | 4641 |
| A574640Hi | 127267 | 4642 |
| A574641Hi | 127268 | 4643 |
| 129001-129500 | | |
| A574642H | 129306 | 4644 |
| A574643H | 129311 | 4645 |
| A574644H | 129312 | 4646 |
| A574645H | 129312 | 4647 |
| A574646H | 129313 | 4648 |
| A574647H | 129314 | 4649 |
| A574648H | 129315 | 4650 |
| A574649H | 129316 | 4651 |
| A574650H | 129316 | 4652 |
| A574651H | 129318 | 4653 |
| A574652H | 129319 | 4654 |
| A574653H | 129319 | 4655 |
| A574654H | 129320 | 4656 |
| A574655H | 129321 | 4657 |
| A574656H | 129322 | 4658 |
| A574657H | 129323 | 4659 |
| A574658H | 129323 | 4660 |
| A574659H | 129324 | 4661 |
| A574660H | 129324 | 4662 |
| A574661H | 129325 | 4663 |
| A574662H | 129325 | 4664 |
| A574663H | 129326 | 4665 |
| A574664H | 129326 | 4666 |
| A574665H | 129327 | 4667 |
| A574666H | 129328 | 4668 |
| A574667H | 129328 | 4669 |
| A574668H | 129329 | 4670 |
| A574669H | 129329 | 4671 |
| A574670H | 129330 | 4672 |
| A574671H | 129330 | 4673 |
| A574672H | 129331 | 4674 |
| A574673H | 129333 | 4675 |
| A574674H | 129335 | 4676 |
| A574675H | 129378 | 4677 |
| A574676H | 129379 | 4678 |
| A574677H | 129380 | 4679 |
| A574678H | 129380 | 4680 |
| A574679H | 129381 | 4681 |
| A574680H | 129383 | 4682 |
| A574681H | 129384 | 4683 |
| A574682H | 129384 | 4684 |
| A574683H | 129387 | 4685 |
| A574684H | 129391 | 4686 |
| A574685H | 129393 | 4687 |
| A574686H | 129395 | 4688 |
| A574687H | 129396 | 4689 |
| A574688H | 129396 | 4690 |
| A574689H | 129397 | 4691 |
| A574690H | 129398 | 4692 |
| A574691H | 129400 | 4693 |
| A574692H | 129401 | 4694 |
| A574693H | 129401 | 4695 |
| A574694H | 129402 | 4696 |
| A574695H | 129402 | 4697 |
| A574696H | 129403 | 4698 |
| A574697H | 129403 | 4699 |
| A574698H | 129404 | 4700 |
| A574699H | 129404 | 4701 |
| A574700H | 129405 | 4702 |
| A574701H | 129405 | 4703 |
| A574702H | 129406 | 4704 |
| A574703H | 129406 | 4705 |
| A574704H | 129406 | 4706 |
| A574705H | 129407 | 4707 |
| A574706H | 129408 | 4708 |
| A574707H | 129409 | 4709 |
| A574708H | 129409 | 4710 |
| A574709H | 129410 | 4711 |
| A574710H | 129411 | 4712 |
| A574711H | 129425 | 4713 |
| A574712H | 129426 | 4714 |
| A574713H | 129431 | 4715 |
| A574714H | 129432 | 4716 |
| A574715H | 129433 | 4717 |
| A574716H | 129476 | 4718 |
| 129501-130000 | | |
| A574717Hi | 129628 | 4719 |
| A574718Hi | 129730 | 4720 |
| A574719Hi | 129733 | 4721 |
| A574720Hi | 129737 | 4722 |
| A574721Hi | 129878 | 4723 |
| 130001-130500 | | |
| A57224Hi | 130119 | 226 |
| A57225Hi | 130196 | 227 |
| A574722Hi | 130190 | 4724 |
| A574723Hi | 130195 | 4725 |
| A574724Hi | 130403 | 4726 |
| A574725Hi | 130404 | 4727 |
| A574726Hi | 130404 | 4728 |
| 131001-131500 | | |
| A574727H | 131132 | 4729 |
| A574728H | 131133 | 4730 |
| A574729H | 131133 | 4731 |
| A574730H | 131134 | 4732 |
| A574731H | 131134 | 4733 |
| A574732H | 131135 | 4734 |
| A574733H | 131135 | 4735 |
| A574734H | 131136 | 4736 |
| A574735H | 131137 | 4737 |
| A574736H | 131137 | 4738 |
| A574737H | 131138 | 4739 |
| A574738H | 131138 | 4740 |
| A574739H | 131138 | 4741 |
| A574740H | 131139 | 4742 |
| A574741H | 131139 | 4743 |
| A574742H | 131140 | 4744 |
| A574743H | 131141 | 4745 |
| A574744H | 131141 | 4746 |
| A574745H | 131142 | 4747 |
| A574746H | 131142 | 4748 |
| A574747H | 131142 | 4749 |
| A574748H | 131143 | 4750 |
| A574749H | 131143 | 4751 |
| A574750Hi | 131204 | 4752 |
| A574751Hi | 131285 | 4753 |
| A574752Hi | 131288 | 4754 |
| A574753Hi | 131359 | 4755 |
| A574754Hi | 131362 | 4756 |
| A574755Hi | 131363 | 4757 |
| A574756Hi | 131363 | 4758 |
| A574757Hi | 131364 | 4759 |
| A574758Hi | 131381 | 4760 |
| A574759Hi | 131405 | 4761 |
| A574760Hi | 131412 | 4762 |
| A574761Hi | 131472 | 4763 |
| 131501-132000 | | |
| A574762Hi | 131551 | 4764 |
| A574763Hi | 131553 | 4765 |
| A574764Hi | 131641 | 4766 |
| A574765Hi | 131642 | 4767 |
| A574766Hi | 131643 | 4768 |
| A574767Hi | 131778 | 4769 |
| 132501-133000 | | |
| A574768Hi | 132888 | 4770 |
| A574769Hi | 132888 | 4771 |
| 134001-134500 | | |
| A574770Hi | 134155 | 4772 |
| A574771Hi | 134214 | 4773 |
| A574772Hi | 134215 | 4774 |
| A574773H | 134274 | 4775 |
| A574774H | 134279 | 4776 |
| A574775H | 134284 | 4777 |
| A574776H | 134300 | 4778 |
| A574777H | 134301 | 4779 |
| A574778H | 134308 | 4780 |
| A574779H | 134309 | 4781 |
| A574780H | 134313 | 4782 |
| A574781H | 134316 | 4783 |
| A574782H | 134321 | 4784 |
| A574783H | 134322 | 4785 |
| A574784H | 134323 | 4786 |
| A574785H | 134324 | 4787 |
| A574786H | 134325 | 4788 |
| A574787H | 134325 | 4789 |
| A574788H | 134331 | 4790 |
| A574789H | 134354 | 4791 |
| A574790H | 134356 | 4792 |
| A574791H | 134357 | 4793 |
| A574792H | 134358 | 4794 |
| A574793H | 134358 | 4795 |
| A574794H | 134359 | 4796 |
| A574795H | 134360 | 4797 |
| A574796H | 134360 | 4798 |
| A574797H | 134367 | 4799 |
| A574798H | 134372 | 4800 |
| A574799H | 134373 | 4801 |
| A574800H | 134392 | 4802 |
| A574801H | 134395 | 4803 |
| A574802H | 134398 | 4804 |
| A574803H | 134399 | 4805 |
| A574804H | 134399 | 4806 |
| A574805H | 134402 | 4807 |
| A574806H | 134414 | 4808 |
| A574807H | 134415 | 4809 |
| A574808H | 134418 | 4810 |
| A574809H | 134419 | 4811 |
| A574810H | 134419 | 4812 |
| A574811H | 134420 | 4813 |
| A574812H | 134421 | 4814 |
| A574813H | 134421 | 4815 |
| A574814H | 134422 | 4816 |
| A574815H | 134422 | 4817 |
| A574816H | 134423 | 4818 |
| A574817H | 134425 | 4819 |
| A574818H | 134426 | 4820 |
| A574819H | 134429 | 4821 |
| A574820H | 134430 | 4822 |
| A574821H | 134430 | 4823 |
| A574822H | 134431 | 4824 |
| 134501-135000 | | |
| A57226Hi | 134758 | 228 |
| A57227Hi | 134850 | 229 |
| A57228Hi | 134851 | 230 |
| A574823Hi | 134519 | 4825 |
| A574824Hi | 134523 | 4826 |
| A574825Hi | 134529 | 4827 |
| A574826Hi | 134683 | 4828 |
| A574827Hi | 134684 | 4829 |
| A574828Hi | 134750 | 4830 |
| A574829Hi | 134751 | 4831 |
| A574830Hi | 134835 | 4832 |
| A574831Hi | 134836 | 4833 |
| A574832Hi | 134849 | 4834 |
| A574833Hi | 134850 | 4835 |
| A574834Hi | 134852 | 4836 |
| A574835Hi | 134853 | 4837 |
| A574836Hi | 134858 | 4838 |
| A574837Hi | 134926 | 4839 |
| A574838Hi | 134928 | 4840 |
| A574839Hi | 134928 | 4841 |
| A574840Hi | 134930 | 4842 |
| A574841Hi | 134931 | 4843 |
| A574842Hi | 134931 | 4844 |
| A574843Hi | 134972 | 4845 |
| 135001-135500 | | |
| A57229Hi | 135164 | 231 |
| A57248Hi | 135068 | 250 |
| A574844Hi | 135103 | 4846 |
| A574845Hi | 135144 | 4847 |
| A574846Hi | 135147 | 4848 |
| A574847Hi | 135158 | 4849 |
| A574848Hi | 135160 | 4850 |
| A574849Hi | 135161 | 4851 |
| A574850Hi | 135163 | 4852 |
| A574851Hi | 135171 | 4853 |
| A574852Hi | 135307 | 4854 |
| A574853Hi | 135461 | 4855 |
| 135501-136000 | | |
| A57230Hi | 135523 | 232 |
| A574854Hi | 135511 | 4856 |
| A574855Hi | 135524 | 4857 |
| A574856Hi | 135525 | 4858 |
| A574857Hi | 135527 | 4859 |
| A574858Hi | 135528 | 4860 |
| A574859Hi | 135609 | 4861 |
| A574860Hi | 135610 | 4862 |
| A574861Hi | 135612 | 4863 |
| A574862Hi | 135710 | 4864 |
| A574863Hi | 135711 | 4865 |
| A574864Hi | 135780 | 4866 |
| A574865Hi | 135783 | 4867 |
| A574866Hi | 135784 | 4868 |
| A574867Hi | 135785 | 4869 |
| A574868Hi | 135789 | 4870 |
| A574869Hi | 135903 | 4871 |
| A574870Hi | 135908 | 4872 |
| A574871Hi | 135909 | 4873 |
| A574872Hi | 135909 | 4874 |
| 136001-136500 | | |
| A574873Hi | 136044 | 4875 |
| A574874Hi | 136044 | 4876 |
| A574875Hi | 136177 | 4877 |
| A574876Hi | 136178 | 4878 |
| A574877Hi | 136260 | 4879 |
| A574878Hi | 136260 | 4880 |
| A574879Hi | 136261 | 4881 |
| 136501-137000 | | |
| A57231Hi | 136662 | 233 |
| A57232Hi | 136678 | 234 |
| A57233Hi | 136683 | 235 |
| A574880Hi | 136655 | 4882 |
| A574881Hi | 136656 | 4883 |
| A574882Hi | 136659 | 4884 |
| A574883Hi | 136663 | 4885 |
| A574884Hi | 136667 | 4886 |
| A574885Hi | 136672 | 4887 |
| A574886Hi | 136676 | 4888 |
| A574887Hi | 136678 | 4889 |
| A574888Hi | 136682 | 4890 |
| A574889Hi | 136683 | 4891 |
| A574890Hi | 136752 | 4892 |
| A574891Hi | 136759 | 4893 |
| A574892Hi | 136761 | 4894 |
| A574893Hi | 136768 | 4895 |
| A574894Hi | 136772 | 4896 |
| A574895Hi | 136869 | 4897 |
| A574896Hi | 136869 | 4898 |
| A574897Hi | 136870 | 4899 |
| A574898Hi | 136870 | 4900 |
| A574899Hi | 136871 | 4901 |
| A574900Hi | 136894 | 4902 |
| A574901Hi | 136894 | 4903 |
| A574902Hi | 136942 | 4904 |
| A574903Hi | 136944 | 4905 |
| A574904Hi | 136949 | 4906 |
| 137001-137500 | | |
| A574905Hi | 137224 | 4907 |
| A574906Hi | 137226 | 4908 |
| A574907Hi | 137226 | 4909 |
| A574908Hi | 137227 | 4910 |
| A574909Hi | 137343 | 4911 |
| A574910Hi | 137344 | 4912 |
| A574911Hi | 137344 | 4913 |
| A574912Hi | 137345 | 4914 |
| A574913Hi | 137348 | 4915 |
| 137501-138000 | | |
| A57234Hi | 137734 | 236 |
| A57249Hi | 137742 | 251 |
| A574914Hi | 137592 | 4916 |
| A574915Hi | 137593 | 4917 |
| A574916Hi | 137596 | 4918 |
| A574917Hi | 137734 | 4919 |
| A574918Hi | 137736 | 4920 |
| A574919Hi | 137743 | 4921 |
| A574920Hi | 137936 | 4922 |
| A574921Hi | 137937 | 4923 |
| A574922Hi | 137938 | 4924 |
| A574923Hi | 137939 | 4925 |
| 138001-138500 | | |
| A57235Hi | 138335 | 237 |
| A574924Hi | 138151 | 4926 |
| A574925Hi | 138219 | 4927 |
| A574926Hi | 138220 | 4928 |
| A574927Hi | 138220 | 4929 |
| A574928Hi | 138270 | 4930 |
| A574929Hi | 138270 | 4931 |
| A574930Hi | 138271 | 4932 |
| A574931Hi | 138271 | 4933 |
| A574932Hi | 138276 | 4934 |
| A574933Hi | 138277 | 4935 |
| A574934Hi | 138278 | 4936 |
| A574935Hi | 138279 | 4937 |
| A574936Hi | 138280 | 4938 |
| A574937Hi | 138280 | 4939 |
| A574938Hi | 138281 | 4940 |
| A574939Hi | 138339 | 4941 |
| A574940Hi | 138339 | 4942 |
| A574941Hi | 138366 | 4943 |
| A574942Hi | 138366 | 4944 |
| A574943H | 138418 | 4945 |
| A574944H | 138419 | 4946 |
| A574945H | 138441 | 4947 |
| A574946H | 138442 | 4948 |
| A574947H | 138442 | 4949 |
| A574948H | 138480 | 4950 |
| A574949H | 138482 | 4951 |
| A574950H | 138483 | 4952 |
| A574951H | 138484 | 4953 |
| A574952H | 138484 | 4954 |
| A574953H | 138488 | 4955 |
| A574954H | 138489 | 4956 |
| A574955H | 138491 | 4957 |
| A574956H | 138492 | 4958 |
| A574957H | 138492 | 4959 |
| A574958H | 138494 | 4960 |
| A574959H | 138498 | 4961 |
| A574960H | 138499 | 4962 |
| 138501-139000 | | |
| A57236Hi | 138834 | 238 |
| A574961Hi | 138595 | 4963 |
| A574962Hi | 138597 | 4964 |
| A574963Hi | 138686 | 4965 |
| A574964Hi | 138687 | 4966 |
| A574965Hi | 138688 | 4967 |
| A574966Hi | 138864 | 4968 |
| 139001-139500 | | |
| A574967Hi | 139017 | 4969 |
| A574968Hi | 139018 | 4970 |
| A574969Hi | 139019 | 4971 |
| A574970Hi | 139021 | 4972 |
| A574971Hi | 139023 | 4973 |
| A574972Hi | 139026 | 4974 |
| A574973Hi | 139054 | 4975 |
| A574974Hi | 139057 | 4976 |
| A574975Hi | 139058 | 4977 |
| A574976Hi | 139066 | 4978 |
| A574977Hi | 139070 | 4979 |
| A574978Hi | 139108 | 4980 |
| A574979Hi | 139111 | 4981 |
| A574980Hi | 139113 | 4982 |
| A574981Hi | 139302 | 4983 |
| A574982Hi | 139306 | 4984 |
| A574983Hi | 139348 | 4985 |
| A574984Hi | 139398 | 4986 |
| A574985Hi | 139399 | 4987 |
| A574986Hi | 139400 | 4988 |
| 139501-140000 | | |
| A574987Hi | 139600 | 4989 |
| A574988Hi | 139650 | 4990 |
| A574989H | 139867 | 4991 |
| A574990H | 139873 | 4992 |
| A574991H | 139878 | 4993 |
| A574992H | 139879 | 4994 |
| A574993H | 139881 | 4995 |
| A574994H | 139886 | 4996 |
| A574995H | 139891 | 4997 |
| A574996H | 139914 | 4998 |
| A574997H | 139915 | 4999 |
| A574998H | 139915 | 5000 |
| A574999H | 139920 | 5001 |
| A575000H | 139921 | 5002 |
| A575001H | 139922 | 5003 |
| A575002H | 139924 | 5004 |
| A575003H | 139927 | 5005 |
| A575004H | 139928 | 5006 |
| A575005H | 139931 | 5007 |
| A575006H | 139932 | 5008 |
| A575007H | 139936 | 5009 |
| A575008H | 139937 | 5010 |
| A575009H | 139939 | 5011 |
| A575010H | 139942 | 5012 |
| A575011H | 139943 | 5013 |
| A575012H | 139944 | 5014 |
| A575013H | 139944 | 5015 |
| A575014H | 139948 | 5016 |
| A575015H | 139949 | 5017 |
| A575016H | 139951 | 5018 |
| A575017H | 139952 | 5019 |
| A575018H | 139953 | 5020 |
| A575019H | 139953 | 5021 |
| A575020H | 139954 | 5022 |
| A575021H | 139974 | 5023 |
| A575022H | 139974 | 5024 |
| A575023H | 139975 | 5025 |
| A575024H | 139975 | 5026 |
| A575025H | 139975 | 5027 |
| A575026H | 139976 | 5028 |
| A575027H | 139976 | 5029 |
| 140001-140500 | | |
| A575028H | 140022 | 5030 |
| A575029H | 140022 | 5031 |
| A575030H | 140023 | 5032 |
| A575031H | 140023 | 5033 |
| A575032H | 140023 | 5034 |
| A575033H | 140024 | 5035 |
| A575034H | 140024 | 5036 |
| A575035H | 140025 | 5037 |
| A575036H | 140025 | 5038 |
| A575037H | 140026 | 5039 |
| A575038H | 140028 | 5040 |
| A575039H | 140034 | 5041 |
| A575040Hi | 140055 | 5042 |
| A575041Hi | 140083 | 5043 |
| A575042Hi | 140084 | 5044 |
| A575043Hi | 140085 | 5045 |
| A575044Hi | 140254 | 5046 |
| A575045Hi | 140254 | 5047 |
| A575046Hi | 140255 | 5048 |
| A575047Hi | 140257 | 5049 |
| A575048Hi | 140257 | 5050 |
| A575049Hi | 140259 | 5051 |
| A575050Hi | 140261 | 5052 |
| A575051Hi | 140262 | 5053 |
| A575052Hi | 140264 | 5054 |
| A575053Hi | 140393 | 5055 |
| A575054Hi | 140394 | 5056 |
| A575055Hi | 140395 | 5057 |
| A575056Hi | 140416 | 5058 |
| 140501-141000 | | |
| A57237Hi | 140893 | 239 |
| A575057Hi | 140901 | 5059 |
| A575058Hi | 140903 | 5060 |
| A575059Hi | 140904 | 5061 |
| A575060Hi | 140951 | 5062 |
| A575061Hi | 140952 | 5063 |
| A575062Hi | 140956 | 5064 |
| A575063Hi | 140959 | 5065 |
| A575064Hi | 140960 | 5066 |
| A575065Hi | 140966 | 5067 |
| A575066Hi | 140971 | 5068 |
| A575067Hi | 140972 | 5069 |
| A575068Hi | 140975 | 5070 |
| A575069Hi | 140980 | 5071 |
| A575070Hi | 140982 | 5072 |
| A575071Hi | 140983 | 5073 |
| A575072Hi | 140988 | 5074 |
| 141001-141500 | | |
| A575073Hi | 141016 | 5075 |
| A575074Hi | 141016 | 5076 |
| A575075Hi | 141017 | 5077 |
| A575076Hi | 141017 | 5078 |
| A575077Hi | 141018 | 5079 |
| A575078Hi | 141019 | 5080 |
| A575079Hi | 141025 | 5081 |
| A575080Hi | 141126 | 5082 |
| A575081Hi | 141140 | 5083 |
| A575082Hi | 141151 | 5084 |
| A575083Hi | 141181 | 5085 |
| A575084Hi | 141182 | 5086 |
| 141501-142000 | | |
| A57238Hi | 141809 | 240 |
| A575085Hi | 141604 | 5087 |
| A575086Hi | 141609 | 5088 |
| A575087Hi | 141620 | 5089 |
| A575088Hi | 141623 | 5090 |
| A575089Hi | 141624 | 5091 |
| A575090Hi | 141625 | 5092 |
| A575091Hi | 141654 | 5093 |
| A575092Hi | 141670 | 5094 |
| A575093Hi | 141778 | 5095 |
| A575094Hi | 141779 | 5096 |
| A575095Hi | 141802 | 5097 |
| A575096Hi | 141802 | 5098 |
| A575097Hi | 141803 | 5099 |
| A575098Hi | 141805 | 5100 |
| A575099Hi | 141807 | 5101 |
| A575100Hi | 141810 | 5102 |
| A575101Hi | 141898 | 5103 |
| A575102Hi | 141903 | 5104 |
| A575103Hi | 141904 | 5105 |
| A575104Hi | 141905 | 5106 |
| 142001-142500 | | |
| A57239Hi | 142174 | 241 |
| A575105H | 142021 | 5107 |
| A575106H | 142022 | 5108 |
| A575107H | 142023 | 5109 |
| A575108H | 142023 | 5110 |
| A575109H | 142025 | 5111 |
| A575110Hi | 142171 | 5112 |
| A575111Hi | 142243 | 5113 |
| A575112Hi | 142246 | 5114 |
| A575113Hi | 142283 | 5115 |
| A575114Hi | 142290 | 5116 |
| A575115Hi | 142292 | 5117 |
| A575116Hi | 142292 | 5118 |
| A575117Hi | 142293 | 5119 |
| A575118Hi | 142298 | 5120 |
| A575119Hi | 142299 | 5121 |
| A575120Hi | 142300 | 5122 |
| A575121Hi | 142300 | 5123 |
| 142501-143000 | | |
| A575122H | 142648 | 5124 |
| A575123H | 142649 | 5125 |
| A575124H | 142672 | 5126 |
| A575125H | 142673 | 5127 |
| A575126H | 142673 | 5128 |
| A575127H | 142675 | 5129 |
| A575128H | 142676 | 5130 |
| A575129H | 142676 | 5131 |
| A575130H | 142677 | 5132 |
| A575131H | 142680 | 5133 |
| A575132H | 142681 | 5134 |
| A575133H | 142681 | 5135 |
| A575134H | 142682 | 5136 |
| A575135H | 142682 | 5137 |
| A575136H | 142687 | 5138 |
| A575137H | 142689 | 5139 |
| A575138H | 142724 | 5140 |
| A575139H | 142782 | 5141 |
| A575140H | 142851 | 5142 |
| 143001-143500 | | |
| A575141H | 143036 | 5143 |
| A575142H | 143037 | 5144 |
| A575143H | 143037 | 5145 |
| A575144H | 143039 | 5146 |
| A575145H | 143040 | 5147 |
| A575146H | 143040 | 5148 |
| A575147H | 143042 | 5149 |
| A575148H | 143043 | 5150 |
| A575149H | 143044 | 5151 |
| A575150H | 143044 | 5152 |
| A575151H | 143045 | 5153 |
| A575152H | 143045 | 5154 |
| A575153H | 143046 | 5155 |
| A575154H | 143048 | 5156 |
| A575155H | 143056 | 5157 |
| A575156H | 143059 | 5158 |
| A575157H | 143060 | 5159 |
| A575158H | 143060 | 5160 |
| A575159H | 143061 | 5161 |
| A575160H | 143062 | 5162 |
| A575161H | 143062 | 5163 |
| A575162H | 143063 | 5164 |
| A575163H | 143063 | 5165 |
| A575164H | 143064 | 5166 |
| A575165H | 143064 | 5167 |
| A575166H | 143064 | 5168 |
| A575167H | 143067 | 5169 |
| A575168H | 143185 | 5170 |
| A575169H | 143186 | 5171 |
| A575170H | 143235 | 5172 |
| A575171H | 143238 | 5173 |
| A575172H | 143239 | 5174 |
| A575173H | 143289 | 5175 |
| A575174H | 143290 | 5176 |
| A575175H | 143290 | 5177 |
| A575176H | 143329 | 5178 |
| A575177H | 143332 | 5179 |
| A575178H | 143335 | 5180 |
| A575179H | 143366 | 5181 |
| A575180H | 143390 | 5182 |
| A575181H | 143393 | 5183 |
| A575182H | 143395 | 5184 |
| A575183H | 143396 | 5185 |
| A575184H | 143397 | 5186 |
| A575185H | 143398 | 5187 |
| A575186H | 143399 | 5188 |
| A575187H | 143401 | 5189 |
| A575188H | 143403 | 5190 |
| A575189H | 143404 | 5191 |
| A575190H | 143408 | 5192 |
| A575191H | 143409 | 5193 |
| A575192H | 143410 | 5194 |
| 143501-144000 | | |
| A575193H | 143531 | 5195 |
| A575194H | 143536 | 5196 |
| A575195H | 143546 | 5197 |
| A575196H | 143547 | 5198 |
| A575197H | 143547 | 5199 |
| A575198H | 143548 | 5200 |
| A575199H | 143549 | 5201 |
| A575200H | 143549 | 5202 |
| A575201H | 143552 | 5203 |
| A575202H | 143553 | 5204 |
| A575203H | 143553 | 5205 |
| A575204H | 143555 | 5206 |
| A575205H | 143556 | 5207 |
| A575206H | 143557 | 5208 |
| A575207H | 143626 | 5209 |
| A575208H | 143627 | 5210 |
| A575209H | 143631 | 5211 |
| A575210H | 143645 | 5212 |
| A575211H | 143646 | 5213 |
| A575212H | 143648 | 5214 |
| A575213H | 143649 | 5215 |
| A575214H | 143650 | 5216 |
| A575215H | 143651 | 5217 |
| A575216H | 143654 | 5218 |
| A575217H | 143656 | 5219 |
| A575218H | 143657 | 5220 |
| A575219H | 143657 | 5221 |
| A575220H | 143658 | 5222 |
| A575221H | 143658 | 5223 |
| A575222H | 143660 | 5224 |
| A575223H | 143663 | 5225 |
| A575224H | 143709 | 5226 |
| A575225H | 143712 | 5227 |
| A575226H | 143716 | 5228 |
| A575227H | 143717 | 5229 |
| A575228H | 143719 | 5230 |
| A575229H | 143720 | 5231 |
| A575230H | 143721 | 5232 |
| A575231H | 143723 | 5233 |
| A575232H | 143724 | 5234 |
| A575233H | 143725 | 5235 |
| A575234H | 143726 | 5236 |
| A575235H | 143727 | 5237 |
| A575236H | 143728 | 5238 |
| A575237H | 143728 | 5239 |
| A575238H | 143729 | 5240 |
| A575239H | 143756 | 5241 |
| A575240H | 143763 | 5242 |
| A575241H | 143785 | 5243 |
| A575242H | 143785 | 5244 |
| A575243H | 143786 | 5245 |
| A575244H | 143787 | 5246 |
| A575245H | 143788 | 5247 |
| A575246H | 143793 | 5248 |
| A575247H | 143807 | 5249 |
| A575248H | 143813 | 5250 |
| A575249H | 143839 | 5251 |
| A575250H | 143840 | 5252 |
| A575251H | 143841 | 5253 |
| A575252H | 143843 | 5254 |
| A575253H | 143844 | 5255 |
| A575254H | 143845 | 5256 |
| A575255H | 143847 | 5257 |
| A575256H | 143869 | 5258 |
| A575257H | 143873 | 5259 |
| A575258H | 143876 | 5260 |
| A575259H | 143877 | 5261 |
| A575260H | 143877 | 5262 |
| A575261H | 143878 | 5263 |
| A575262H | 143878 | 5264 |
| A575263H | 143879 | 5265 |
| A575264H | 143879 | 5266 |
| A575265H | 143880 | 5267 |
| A575266H | 143880 | 5268 |
| A575267H | 143887 | 5269 |
| A575268H | 143888 | 5270 |
| A575269H | 143935 | 5271 |
| A575270H | 143936 | 5272 |
| A575271H | 143938 | 5273 |
| A575272H | 143939 | 5274 |
| A575273H | 143940 | 5275 |
| A575274H | 143942 | 5276 |
| A575275H | 143948 | 5277 |
| A575276H | 143948 | 5278 |
| A575277H | 143976 | 5279 |
| A575278H | 143976 | 5280 |
| A575279H | 143977 | 5281 |
| A575280H | 143978 | 5282 |
| A575281H | 143979 | 5283 |
| A575282H | 143979 | 5284 |
| A575283H | 143980 | 5285 |
| A575284H | 143980 | 5286 |
| A575285H | 143982 | 5287 |
| A575286H | 143983 | 5288 |
| 144001-144173 | | |
| A575287H | 144013 | 5289 |
| A575288H | 144161 | 5290 |
| A575289H | 144163 | 5291 |
| A575290H | 144164 | 5292 |
| A575291H | 144165 | 5293 |
| A575292H | 144171 | 5294 |
| A575293H | 144173 | 5295 |

The oligonucleotides of the present invention hybridize, for example, with mRNA and/or pre-mRNA of human LRRK2 of SEQ ID NO. 1 and/or SEQ ID NO. 2. Such oligonucleotides are called LRRK2 antisense oligonucleotides. Oligonucleotides of the present invention, which are for example antisense oligonucleotides, are shown in Table 1. The present invention further refers to oligonucleotides such as antisense oligonucleotides having about 80% to 99%, about 85% to 98%, about 90% to 95%, about 90% to 99%, about 93% to about 99%, about 94% to about 99%, about 95% to about 99%, at least about 85%, 86%, 87%, 88%, 89%, 90%, 91%, 92%, 93%, 94%, 95%, 96%, 97%, 98%, or 99% sequence homology to an oligonucleotide of Table 1.

Moreover, the present invention refers to oligonucleotides such as antisense oligonucleotides comprising or consisting of at least 7, at least 8, at least 9, at least 10, at least 11, at least 12, at least 13, at least 14, at least 15, at least 16, at least 17, at least 18, at least 19, at least 20, at least 21 or at least 22 nucleotides of any one of the oligonucleotides of SEQ ID NO. 3-SEQ ID NO. 5301.

Each nucleotide of the sequence can be modified, wherein ASOs of the present invention preferably comprise a core of to 8 unmodified nucleotides. ASOs of the present invention comprise, for example, one or more modified nucleotides, e.g., 1, 2, 3, 4 or 5 nucleotides at the 5′- and/or 3′-end of the oligonucleotide, i.e., on the 5′- and/or 3′-side of the core. The 5′- and 3′-end can be modified identically or differently. If the 5′- and 3′-ends are modified identically the nucleotides are modified at the same positions counted from the 5′- and 3′-end (in each case starting the counting with 1 from the end), respectively, having the same modification for example LNA-modification. If the 5′- and 3′-ends are modified differently the position of the modified nucleotide and/or the type of modification at the 5′- and 3′-ends differ; the type of nucleotide modification is the same (e.g., LNA) or different. Modified nucleotides such as LNA-modified nucleotides need not to follow in a row, but may be separated by one or more unmodified nucleotides. In the following table exemplary modification patterns at the 5′- and 3′-end of the ASOs of the present invention are described, wherein an unmodified nucleotide is indicated by “_” and the number on either side of the “_” refers to the number of modified nucleotides such as LNA-modified nucleotides in a row. The modified nucleotide(s) is/are at any position of the 5′- and/or 3′-end of the ASO as shown in the following Table 3:

TABLE 3

| LNA modification at the | LNA modification at the | |
|---|---|---|
| 5′-side of the core | 3′-side of the core | Abbreviation |
| 3 | 3 | 3 + 3 |
| 3 | 2 | 3 + 2 |
| 2 | 3 | 2 + 3 |
| 1_1 | 3 | 1_1 + 3 |
| 1_1 | 2 | 1_1 + 2 |
| 1_1 | 1_1 | 1_1 + 1_1 |
| 3 | 1_1 | 3 + 1_1 |
| 2 | 1_1 | 2 + 1_1 |
| 2 | 2 | 2 + 2 |
| 4 | 3 | 4 + 3 |
| 4 | 2 | 4 + 2 |
| 4 | 1_1 | 4 + 1_1 |
| 2_1 | 3 | 2_1 + 3 |
| 2_1 | 1_1 | 2_1 + 1_1 |
| 2_1 | 2 | 2_1 + 2 |
| 3 | 4 | 3 + 4 |
| 2 | 4 | 2 + 4 |
| 1_1 | 4 | 1_1 + 4 |
| 3 | 1_2 | 3 + 1_2 |
| 1_1 | 1_2 | 1_1 + 1_2 |
| 2 | 1_2 | 2 + 1_2 |

which indicates specific positions of the LNA modifications at the 5′- and 3X-end of each ASO in Table 4:

TABLE 4

| Position of the | Position of the | |
|---|---|---|
| modification at the 5′-end | modification at the 3′-end | |
| (counted from the 5′-end | (counted from the 3′-end | |
| starting with 1) | starting with 1) | Abbreviation |
| nucleotides 1 to 5 | nucleotides 1 to 5 | 5 + 5 |
| nucleotides 1 to 4 | nucleotides 1 to 4 | 4 + 4 |
| nucleotides 1 to 3 | nucleotides 1 to 3 | 3 + 3 |
| nucleotides 1 and 2 | nucleotides 1 and 2 | 2 + 2 |
| nucleotide 1 | nucleotide 1 | 1 + 1 |
| nucleotides 1 to 5 | nucleotides 1 to 4 | 5 + 4 |
| nucleotides 1 to 4 | nucleotides 1 to 3 | 4 + 3 |
| nucleotides 1 to 3 | nucleotides 1 and 2 | 3 + 2 |
| nucleotides 1 and 2 | nucleotide 1 | 2 + 1 |
| nucleotide 1 | nucleotides 1 to 5 | 1 + 5 |
| nucleotides 1 to 5 | nucleotides 1 to 3 | 5 + 3 |
| nucleotides 1 to 4 | nucleotides 1 and 2 | 4 + 2 |
| nucleotides 1 to 3 | nucleotide 1 | 3 + 1 |
| nucleotides 1 and 2 | nucleotides 1 to 5 | 2 + 5 |
| nucleotide 1 | nucleotides 1 to 4 | 1 + 4 |
| nucleotides 1 to 5 | nucleotides 1 and 2 | 5 + 2 |
| nucleotides 1 to 4 | nucleotide 1 | 4 + 1 |
| nucleotides 1 to 3 | nucleotides 1 to 5 | 3 + 5 |
| nucleotides 1 and 2 | nucleotides 1 to 4 | 2 + 4 |
| nucleotide 1 | nucleotides 1 to 3 | 1 + 3 |
| nucleotides 1 to 5 | nucleotide 1 | 5 + 1 |
| nucleotides 1 to 4 | nucleotides 1 to 5 | 4 + 5 |
| nucleotides 1 to 3 | nucleotides 1 to 4 | 3 + 4 |
| nucleotides 1 and 2 | nucleotides 1 to 3 | 2 + 3 |
| nucleotide 1 | nucleotides 1 and 2 | 1 + 2 |
| nucleotides 1 and 2 | nucleotides 1 and 3 | 2 + 1_1 |
| nucleotides 1 and 3 | nucleotides 1 to 3 | 1_1 + 3 |
| nucleotides 1 and 3 | nucleotides 1 and 3 | 1_1 + 1_1 |
| nucleotides 1 to 3 | nucleotides 1 and 3 | 3 + 1_1 |
| nucleotides 1 to 4 | nucleotides 1 and 3 | 4 + 1_1 |
| nucleotides 1, 2 and 4 | nucleotides 1 to 3 | 2_1 + 3 |
| nucleotides 1, 2 and 4 | nucleotides 1 and 3 | 2_1 + 1_1 |
| nucleotides 1, 2 and 4 | nucleotides 1 and 2 | 2_1 + 2 |
| nucleotides 1 and 3 | nucleotides 1 to 4 | 1_1 + 4 |
| nucleotides 1 to 3 | nucleotides 1, 2 and 4 | 3 + 1_2 |
| nucleotides 1 and 3 | nucleotides 1, 2 and 4 | 1_1 + 1_2 |
| nucleotides 1 and 2 | nucleotides 1, 2 and 4 | 2 + 1_2 |

In some embodiments, the oligonucleotide of the present invention can reduce the amount of LRRK2 mRNA and/or the LRRK2 protein expression for example by about 30%-100%, 35%-99%, 40%-98%, 45%-97%, 50%-96%, 55%-95%, 60%-90%, 65%-85%, 70%-80% or at least about 30%, 35%, 40%, 45%, 50%, 55%, 60%, 65%, 70%, 75%, 80%, 85%, 90%, 92%, 94%, 95%, 96%, 97%, 98%, 99% or 100%. The reduction of the amount of the LRRK2 mRNA and/or LRRK2 protein expression can be determined by the comparison of the amount of the LRRK2 mRNA and/or LRRK2 protein expression in a sample treated with an oligonucleotide of the present invention and a corresponding untreated control. The untreated control is, for example, LRRK2, LRRK2 mRNA, LRRK2 pre-mRNA expression, or a combination thereof in a subject prior to administration of an oligonucleotide of the present invention is administered or an untreated sample such as a cell, blood, urine, saliva etc. The untreated sample is, for example, taken from a subject before an oligonucleotide of the present invention is administered.

The oligonucleotide of the present invention reduces the amount of LRRK2 mRNA and/or the expression of LRRK2 protein expression at a nanomolar or micromolar concentration for example at a concentration of about 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

The oligonucleotide of the present invention is, for example, used in a concentration of about 1, 3, 5, 9, 10, 15, 27, 30, 40, 50, 75, 82, 100, 250, 300, 500, or 740 nM, or about 1, 2.2, 3, 5, 6.6 or 10 μM.

The present invention also refers to a pharmaceutical composition comprising an oligonucleotide described herein and a pharmaceutically acceptable carrier, excipient, stimulant, adjuvant, and/or dilutant. Optionally, the pharmaceutical composition further comprises another oligonucleotide which is different from the present invention, an antibody and/or a small molecule such as a therapeutically active agent for use in preventing and/or treating a human disease, for example PD—a prevalent and severe neurodegenerative disease.

The oligonucleotide or the pharmaceutical composition of the present invention is, for example, for use in a method of preventing and/or treating a disorder such as a neurodegenerative disorder. The neurodegenerative disorder is, for example, Parkinson's disease, Alzheimer's disease, Huntington's disease, amyotrophic lateral sclerosis, multiple sclerosis, multiple system atrophy, dementia with Lewy bodies (DLB), pure autonomic failure (PAF), frontotemporal dementia or prion diseases.

The disorder is, for example, characterized by activity of LRRK2, such as overactivity or a LRRK2 mRNA and/or protein imbalance, i.e., the LRRK2 mRNA and/or protein level is abnormal (e.g., increased) in comparison to the level in a normal, healthy cell, tissue, organ or subject, or LRRK2 activity is abnormal. The LRRK2 level is, for example, increased by an increased amount of LRRK2 mRNA and/or LRRK2 protein expression. Overactivity of LRRK2 function is, for example, caused by activating mutations or by unknown genetic, epigenetic or environmental mechanisms.

The LRRK2 mRNA and protein level, respectively, can be measured by any standard method known to a person skilled in the art, such as immunohistochemistry, western blot, quantitative real time PCR or QUANTIGENE assay known to a person skilled in the art.

An oligonucleotide or a pharmaceutical composition described herein can be administered locally or systemically for example orally, sublingually, nasally, subcutaneously, intravenously, intraperitoneally, intramuscularly, intratumorally, intrathecally, intraventricularly, transdermal and/or rectally. The oligonucleotide is, for example, administered alone or in combination with another oligonucleotide of the present invention, and optionally in combination with another compound such as another oligonucleotide different from the present invention, an antibody, and/or a small molecule.

Further, two or more oligonucleotides of the present invention are, for example, administered together, at the same time point, e.g., in a pharmaceutical composition or separately, or on staggered intervals. Alternatively or in addition, one or more oligonucleotides described herein can be administered together with another compound such as another oligonucleotide (i.e., different from the present invention), an antibody, and/or a small molecule, at the same time point for example in a pharmaceutical composition or separately, or on staggered intervals. In some of these combinations, the oligonucleotide of the present invention reduces, for example, the amount of LRRK2 mRNA and/or LRRK2 protein expression and the other oligonucleotide (i.e., different from the present invention), the antibody and/or small molecule inhibits (antagonist) or stimulates (agonist) a further factor involved in formation or progression of neurodegenerative diseases. This factor is, for example, α-synuclein, Tau, TDO, NLRP3, PFKB3 or a combination thereof. The factor can be a factor whose level or activity is increased or decreased in a cell, tissue, organ or subject depending on the cell, tissue, organ or subject and its individual conditions.

The oligonucleotide of the present invention is, for example, combined with another therapeutically active agent. Suitable therapeutically active agents include, but are not limited to L-dopa, a dopamine agonist, monoamine oxidase inhibitor, etc. known to those skilled in the art.

An antibody in combination with the oligonucleotide or the pharmaceutical composition of the present invention is, for example, an anti-α-synuclein such as Prasinezumab (RO7046015/PRX002), Cinpanemab, ABBV-805, MEDI1341 or LU AF82422 antibody.

A subject of the present invention is for example a mammalian (e.g., a human, dog, cat horse, cow, pig), bird, or fish.

### EXAMPLES

The following examples illustrate non-limiting embodiments of the present invention, but the invention is not limited to these examples. The following experiments were performed on cells endogenously expressing LRRK2, i.e., the cells do not represent an artificial system comprising transfected reporter constructs. Such artificial systems generally show a higher degree of inhibition and lower IC₅₀ values than endogenous systems which are closer to therapeutically relevant in vivo systems. Further, in the following experiments no transfecting agent were used, i.e., gymnotic delivery was performed. Transfecting agents are known to increase the activity of an oligonucleotide which influences artificially the IC₅₀ value (see for example Zhang et al., Gene Therapy, 2011, 18, 326-333; Stanton et al., Nucleic Acid Therapeutics, Vol. 22, No. 5, 2012). Since artificial systems using a transfecting agent are hard or impossible to translate into therapeutic approaches and no transfection formulation has been approved so far for oligonucleotides, the following experiments were performed without any transfecting agent.

### Example 1: Target Knockdown Efficacy Screens of Human LRRK2-Specific ASOs in A549 and MDA-MB-231 Cells

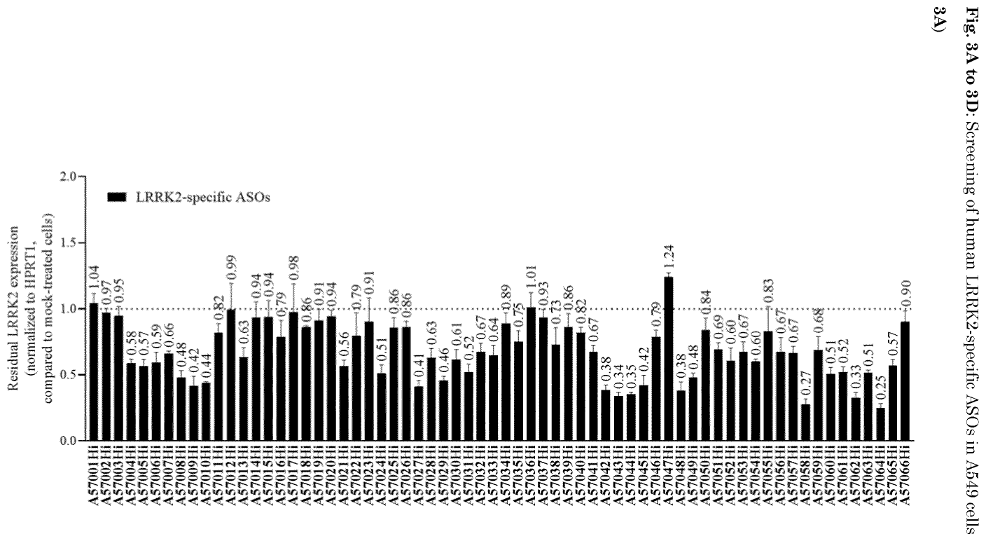

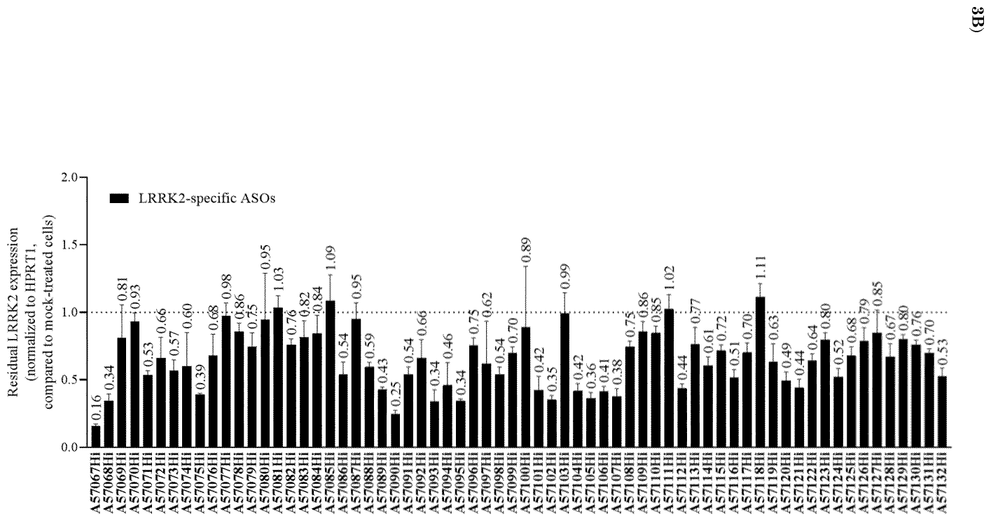

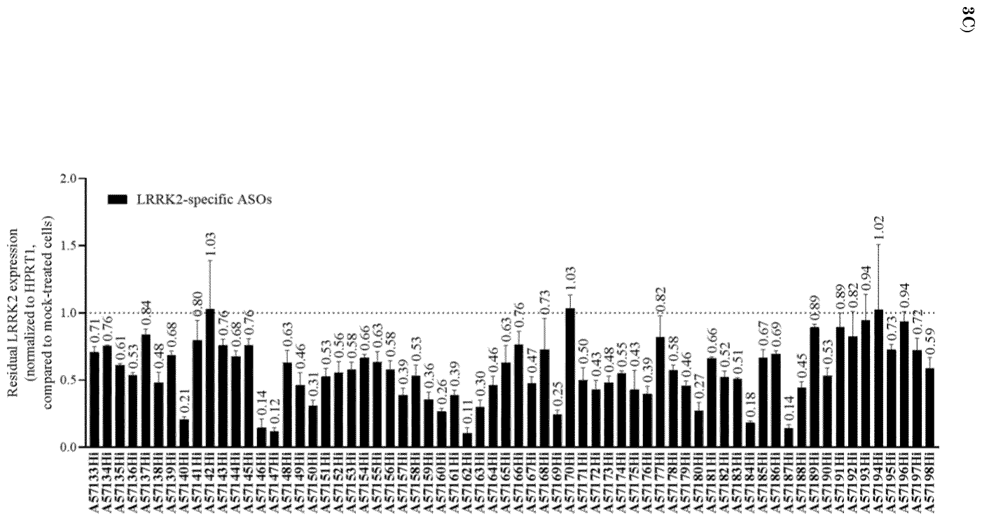

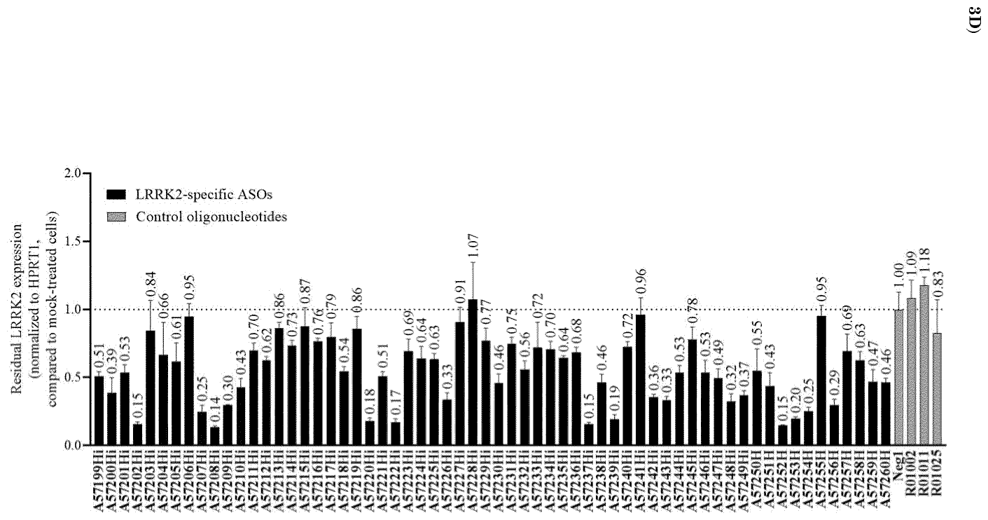

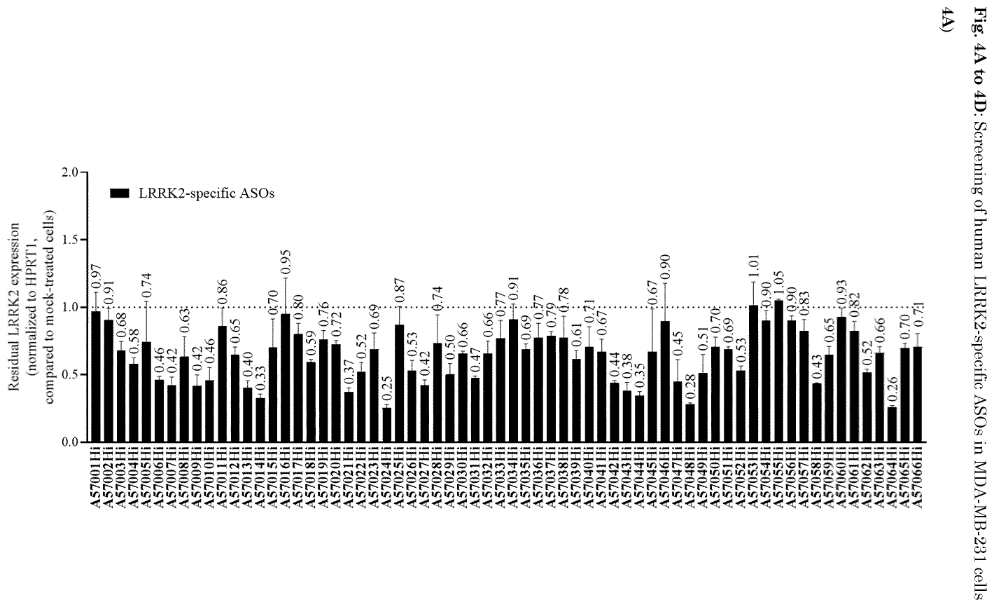

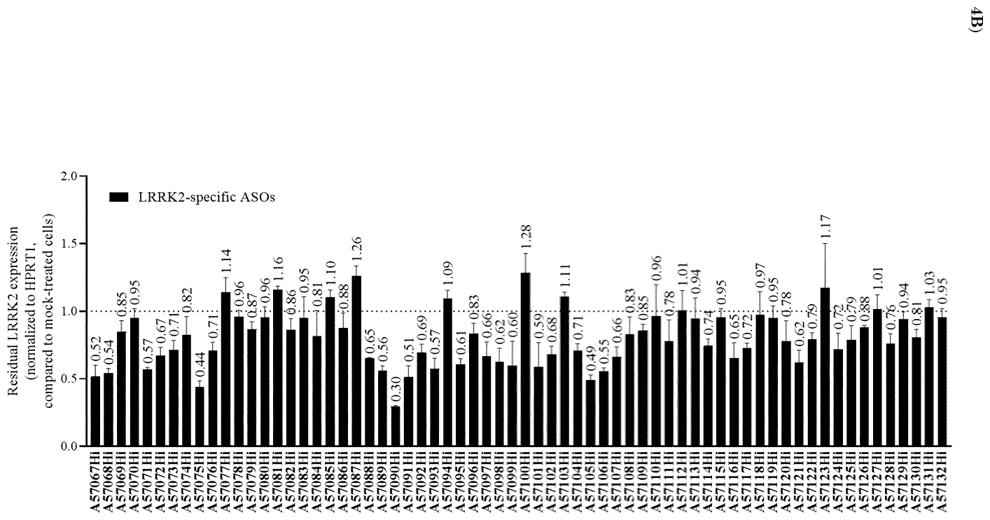

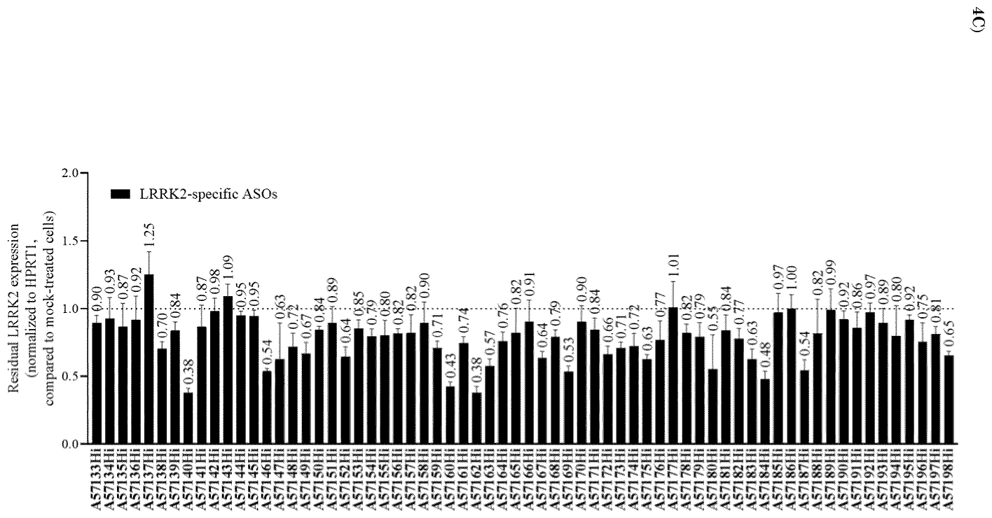

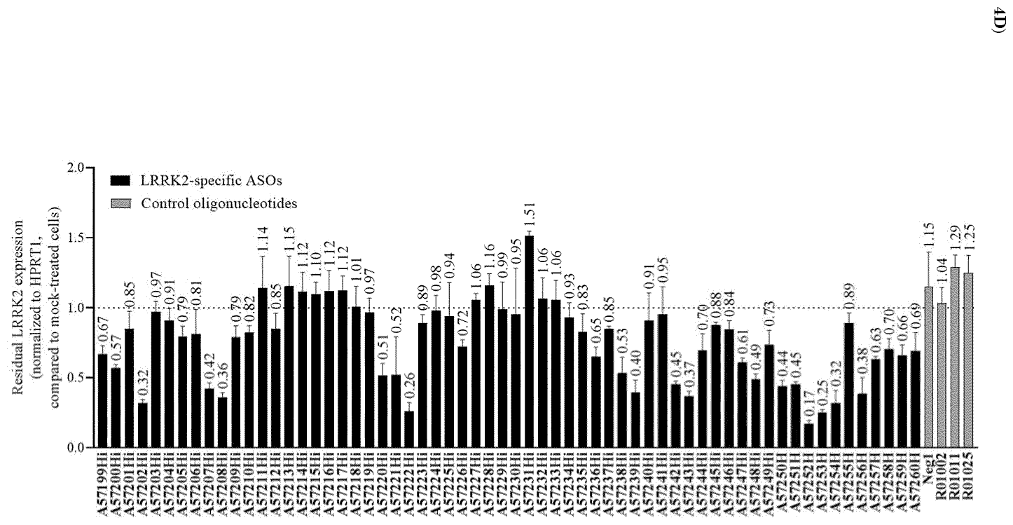

Knockdown efficacy of the LRRK2-specific ASOs was tested in human A549 cells and MDA-MB-231 cells. The cells were treated with the respective LRRK2-specific ASO or control oligonucleotide at a concentration of 5 μM without the use of a transfection reagent. After three days of treatment, cells were lyzed. LRRK2 and HPRT1 mRNA expression was analyzed using the QUANTIGENE SINGLEPLEX assay (THERMOFISHER) and the LRRK2 expression values were normalized to HPRT1 values. The results of ASOs in A549 cells and in MDA-MB-231 cells are shown as residual LRRK2 mRNA expression relative to mock-treated cells (set as 1) in FIGS. 3A to 3D, Table 5 and FIGS. 4A to 4D. Treatment of A549 cells with the ASOs A57162Hi (SEQ ID NO. 164), A57147Hi (SEQ ID NO. 149), A57208Hi (SEQ ID NO. 210), A57187Hi (SEQ ID NO. 189), A57146Hi (SEQ ID NO. 148), A575302Hi (SEQ ID NO. 69), A575307Hi (SEQ ID NO. 69), A575308Hi (SEQ ID NO. 69), A575309Hi (SEQ ID NO. 69), A575310Hi (SEQ ID NO. 69), A575313Hi (SEQ ID NO. 69), A575321Hi (SEQ ID NO. 142), A575323Hi (SEQ ID NO. 142), A575326Hi (SEQ ID NO. 142), A575327Hi (SEQ ID NO. 142), A575335Hi (SEQ ID NO. 209), A575337Hi (SEQ ID NO. 209), A575338Hi (SEQ ID NO. 209), A575341Hi (SEQ ID NO. 209), A575349Hi, A575351Hi (SEQ ID NO. 210), A575354Hi (SEQ ID NO. 210), and A575355Hi (SEQ ID NO. 209), respectively, resulted in a target inhibition of >85% (represented by a residual LRRK2 mRNA expression of <0.15 as compared to mock-treated cells) (FIGS. 3A to 3D). As shown in FIGS. 4A to 4D, treatment with the ASOs A57252H (SEQ ID NO. 254), A57253H (SEQ ID NO. 255) and A57024Hi (SEQ ID NO. 26) resulted in a target inhibition of ≥75% (represented by a residual LRRK2 mRNA expression of 0.25 as compared to mock treated cells). Table 5 shows mean residual expression of human LRRK2 (normalized to HPRT1 and compared to mock-treated cells) in A549 cells after ASO treatment:

| | Mean residual expression of human LRRK2 |
|---|---|
| | (normalized to HPRT1 and compared to |
| Name | mock-treated cells) |
| A57001Hi | 1.04 |
| A57002Hi | 0.97 |
| A57003Hi | 0.95 |
| A57004Hi | 0.58 |
| A57005Hi | 0.57 |
| A57006Hi | 0.59 |
| A57007Hi | 0.66 |
| A57008Hi | 0.48 |
| A57009Hi | 0.42 |
| A57010Hi | 0.44 |
| A57011Hi | 0.82 |
| A57012Hi | 0.99 |
| A57013Hi | 0.63 |
| A57014Hi | 0.94 |
| A57015Hi | 0.94 |
| A57016Hi | 0.79 |
| A57017Hi | 0.98 |
| A57018Hi | 0.86 |
| A57019Hi | 0.91 |
| A57020Hi | 0.94 |
| A57021Hi | 0.56 |
| A57022Hi | 0.79 |
| A57023Hi | 0.91 |
| A57024Hi | 0.51 |
| A57025Hi | 0.86 |
| A57026Hi | 0.86 |
| A57027Hi | 0.41 |
| A57028Hi | 0.63 |
| A57029Hi | 0.46 |
| A57030Hi | 0.61 |
| A57031Hi | 0.52 |
| A57032Hi | 0.67 |
| A57033Hi | 0.64 |
| A57034Hi | 0.89 |
| A57035Hi | 0.75 |
| A57036Hi | 1.01 |
| A57037Hi | 0.93 |
| A57038Hi | 0.73 |
| A57039Hi | 0.86 |
| A57040Hi | 0.82 |
| A57041Hi | 0.67 |
| A57042Hi | 0.38 |
| A57043Hi | 0.34 |
| A57044Hi | 0.35 |
| A57045Hi | 0.42 |
| A57046Hi | 0.79 |
| A57047Hi | 1.24 |
| A57048Hi | 0.38 |
| A57049Hi | 0.48 |
| A57050Hi | 0.84 |
| A57051Hi | 0.69 |
| A57052Hi | 0.60 |
| A57053Hi | 0.67 |
| A57054Hi | 0.60 |
| A57055Hi | 0.83 |
| A57056Hi | 0.67 |
| A57057Hi | 0.67 |
| A57058Hi | 0.27 |
| A57059Hi | 0.68 |
| A57060Hi | 0.51 |
| A57061Hi | 0.52 |
| A57062Hi | 0.33 |
| A57063Hi | 0.51 |
| A57064Hi | 0.25 |
| A57065Hi | 0.57 |
| A57066Hi | 0.90 |
| A57067Hi | 0.16 |
| A57068Hi | 0.34 |
| A57069Hi | 0.81 |
| A57070Hi | 0.93 |
| A57071Hi | 0.53 |
| A57072Hi | 0.66 |
| A57073Hi | 0.57 |
| A57074Hi | 0.60 |
| A57075Hi | 0.39 |
| A57076Hi | 0.68 |
| A57077Hi | 0.98 |
| A57078Hi | 0.86 |
| A57079Hi | 0.75 |
| A57080Hi | 0.95 |
| A57081Hi | 1.03 |
| A57082Hi | 0.76 |
| A57083Hi | 0.82 |
| A57084Hi | 0.84 |
| A57085Hi | 1.09 |
| A57086Hi | 0.54 |
| A57087Hi | 0.95 |
| A57088Hi | 0.59 |
| A57089Hi | 0.43 |
| A57090Hi | 0.25 |
| A57091Hi | 0.54 |
| A57092Hi | 0.66 |
| A57093Hi | 0.34 |
| A57094Hi | 0.46 |
| A57095Hi | 0.34 |
| A57096Hi | 0.75 |
| A57097Hi | 0.62 |
| A57098Hi | 0.54 |
| A57099Hi | 0.70 |
| A57100Hi | 0.89 |
| A57101Hi | 0.42 |
| A57102Hi | 0.35 |
| A57103Hi | 0.99 |
| A57104Hi | 0.42 |
| A57105Hi | 0.36 |
| A57106Hi | 0.41 |
| A57107Hi | 0.38 |
| A57108Hi | 0.75 |
| A57109Hi | 0.86 |
| A57110Hi | 0.85 |
| A57111Hi | 1.02 |
| A57112Hi | 0.44 |
| A57113Hi | 0.77 |
| A57114Hi | 0.61 |
| A57115Hi | 0.72 |
| A57116Hi | 0.51 |
| A57117Hi | 0.70 |
| A57118Hi | 1.11 |
| A57119Hi | 0.63 |
| A57120Hi | 0.49 |
| A57121Hi | 0.44 |
| A57122Hi | 0.64 |
| A57123Hi | 0.80 |
| A57124Hi | 0.52 |
| A57125Hi | 0.68 |
| A57126Hi | 0.79 |
| A57127Hi | 0.85 |
| A57128Hi | 0.67 |
| A57129Hi | 0.80 |
| A57130Hi | 0.76 |
| A57131Hi | 0.70 |
| A57132Hi | 0.52 |
| A57133Hi | 0.71 |
| A57134Hi | 0.76 |
| A57135Hi | 0.61 |
| A57136Hi | 0.53 |
| A57137Hi | 0.84 |
| A57138Hi | 0.48 |
| A57139Hi | 0.68 |
| A57140Hi | 0.21 |
| A57141Hi | 0.80 |
| A57142Hi | 1.03 |
| A57143Hi | 0.76 |
| A57144Hi | 0.68 |
| A57145Hi | 0.76 |
| A57146Hi | 0.14 |
| A57147Hi | 0.12 |
| A57148Hi | 0.63 |
| A57149Hi | 0.46 |
| A57150Hi | 0.31 |
| A57151Hi | 0.53 |
| A57152Hi | 0.56 |
| A57153Hi | 0.58 |
| A57154Hi | 0.66 |
| A57155Hi | 0.63 |
| A57156Hi | 0.58 |
| A57157Hi | 0.39 |
| A57158Hi | 0.53 |
| A57159Hi | 0.36 |
| A57160Hi | 0.26 |
| A57161Hi | 0.39 |
| A57162Hi | 0.11 |
| A57163Hi | 0.30 |
| A57164Hi | 0.46 |
| A57165Hi | 0.63 |
| A57166Hi | 0.76 |
| A57167Hi | 0.47 |
| A57168Hi | 0.73 |
| A57169Hi | 0.25 |
| A57170Hi | 1.03 |
| A57171Hi | 0.50 |
| A57172Hi | 0.43 |
| A57173Hi | 0.48 |
| A57174Hi | 0.55 |
| A57175Hi | 0.43 |
| A57176Hi | 0.39 |
| A57177Hi | 0.82 |
| A57178Hi | 0.58 |
| A57179Hi | 0.46 |
| A57180Hi | 0.27 |
| A57181Hi | 0.66 |
| A57182Hi | 0.52 |
| A57183Hi | 0.51 |
| A57184Hi | 0.18 |
| A57185Hi | 0.67 |
| A57186Hi | 0.69 |
| A57187Hi | 0.14 |
| A57188Hi | 0.45 |
| A57189Hi | 0.89 |
| A57190Hi | 0.53 |
| A57191Hi | 0.89 |
| A57192Hi | 0.82 |
| A57193Hi | 0.94 |
| A57194Hi | 1.02 |
| A57195Hi | 0.73 |
| A57196Hi | 0.94 |
| A57197Hi | 0.72 |
| A57198Hi | 0.59 |
| A57199Hi | 0.51 |
| A57200Hi | 0.39 |
| A57201Hi | 0.53 |
| A57202Hi | 0.15 |
| A57203Hi | 0.84 |
| A57204Hi | 0.66 |
| A57205Hi | 0.61 |
| A57206Hi | 0.95 |
| A57207Hi | 0.25 |
| A57208Hi | 0.14 |
| A57209Hi | 0.30 |
| A57210Hi | 0.43 |
| A57211Hi | 0.70 |
| A57212Hi | 0.62 |
| A57213Hi | 0.86 |
| A57214Hi | 0.73 |
| A57215Hi | 0.87 |
| A57216Hi | 0.76 |
| A57217Hi | 0.79 |
| A57218Hi | 0.54 |
| A57219Hi | 0.86 |
| A57220Hi | 0.18 |
| A57221Hi | 0.51 |
| A57222Hi | 0.17 |
| A57223Hi | 0.69 |
| A57224Hi | 0.64 |
| A57225Hi | 0.63 |
| A57226Hi | 0.33 |
| A57227Hi | 0.91 |
| A57228Hi | 1.07 |
| A57229Hi | 0.77 |
| A57230Hi | 0.46 |
| A57231Hi | 0.75 |
| A57232Hi | 0.56 |
| A57233Hi | 0.72 |
| A57234Hi | 0.70 |
| A57235Hi | 0.64 |
| A57236Hi | 0.68 |
| A57237Hi | 0.15 |
| A57238Hi | 0.46 |
| A57239Hi | 0.19 |
| A57240Hi | 0.72 |
| A57241Hi | 0.96 |
| A57242Hi | 0.36 |
| A57243Hi | 0.33 |
| A57244Hi | 0.53 |
| A57245Hi | 0.78 |
| A57246Hi | 0.53 |
| A57247Hi | 0.49 |
| A57248Hi | 0.32 |
| A57249Hi | 0.37 |
| A57250H | 0.55 |
| A57251H | 0.43 |
| A57252H | 0.15 |
| A57253H | 0.20 |
| A57254H | 0.25 |
| A57255H | 0.95 |
| A57256H | 0.29 |
| A57257H | 0.69 |
| A57258H | 0.63 |
| A57259H | 0.47 |
| A57260H | 0.46 |
| A575301Hi | 0.29 |
| A575302Hi | 0.14 |
| A575304Hi | 0.38 |
| A575307Hi | 0.06 |
| A575308Hi | 0.11 |
| A575309Hi | 0.07 |
| A575310Hi | 0.07 |
| A575312Hi | 0.17 |
| A575313Hi | 0.08 |
| A575315Hi | 0.24 |
| A575316Hi | 0.22 |
| A575318Hi | 0.31 |
| A575321Hi | 0.12 |
| A575322Hi | 0.20 |
| A575323Hi | 0.09 |
| A575324Hi | 0.18 |
| A575326Hi | 0.11 |
| A575327Hi | 0.10 |
| A575329Hi | 0.27 |
| A575330Hi | 0.28 |
| A575332Hi | 0.25 |
| A575335Hi | 0.07 |
| A575336Hi | 0.21 |
| A575337Hi | 0.09 |
| A575338Hi | 0.07 |
| A575340Hi | 0.17 |
| A575341Hi | 0.04 |
| A575343Hi | 0.33 |
| A575344Hi | 0.15 |
| A575346Hi | 0.22 |
| A575349Hi | 0.05 |
| A575350Hi | 0.16 |
| A575351Hi | 0.10 |
| A575352Hi | 0.19 |
| A575354Hi | 0.14 |
| A575355Hi | 0.11 |
| Neg1 | 1.00 |
| R01002 | 1.09 |
| R01011 | 1.18 |
| R01025 | 0.83 |

### Example 2: Concentration-Response Curves and Determination of IC₅₀ Values of Selected Human LRRK2-Specific ASOs in A549 Cells

Knockdown efficacy of selected ASO was tested in human A549 cells at different concentrations. Therefore, cells were treated with the respective LRRK2 ASO at the following concentrations: 10000 nM, 3333 nM, 1111 nM, 370 nM, 123 nM, 41 nM, 14 nM without the use of a transfection reagent. After three days of treatment, cells were lyzed. LRRK2 and HPRT1 mRNA expression was analyzed using the QUANTIGENE SINGLEPLEX assay (THERMOFISHER) and the LRRK2 expression values were normalized to HPRT1 values. Concentration-dependent knockdown of LRRK2 mRNA expression is shown as residual LRRK2 mRNA expression normalized to HPRT1 mRNA expression relative to mock-treated cells (set as 1) in the following Table 6:

| | ASO concentration | | | | | | | IC₅₀ value | SEQ |
|---|---|---|---|---|---|---|---|---|---|
| ASO ID | 14 nM | 41 nM | 123 nM | 370 nM | 1111 nM | 3333 nM | 10000 nM | (nM) | ID NO. |
| A57043Hi | 1.11 | 0.98 | 1.05 | 0.82 | 0.56 | 0.37 | 0.25 | 883 | 45 |
| A57044Hi | 1.07 | 0.99 | 0.79 | 0.92 | 0.57 | 0.49 | 0.30 | 1530 | 46 |
| A57058Hi | 1.01 | 0.71 | 0.79 | 0.71 | 0.52 | 0.36 | 0.28 | 1438 | 60 |
| A57064Hi | 0.95 | 0.72 | 0.85 | 0.74 | 0.44 | 0.31 | 0.19 | 1586 | 66 |
| A57067Hi | 1.01 | 0.95 | 0.77 | 0.68 | 0.39 | 0.20 | 0.12 | 622 | 69 |
| A57140Hi | 0.89 | 0.85 | 0.84 | 0.67 | 0.49 | 0.29 | 0.15 | 2207 | 142 |
| A57160Hi | 0.87 | 0.80 | 0.93 | 0.80 | 0.48 | 0.30 | 0.21 | 847 | 162 |
| A57162Hi | 0.96 | 0.86 | 0.67 | 0.61 | 0.40 | 0.15 | 0.11 | 646 | 164 |
| A57169Hi | 1.00 | 0.87 | 0.83 | 0.75 | 0.62 | 0.38 | 0.19 | 888 | 171 |
| A57184Hi | n.d. | 0.93 | 0.89 | 0.79 | 0.53 | 0.26 | 0.14 | 1169 | 186 |
| A57202Hi | 0.98 | 0.94 | 0.86 | 0.73 | 0.43 | 0.27 | 0.15 | 759 | 204 |
| A57207Hi | 0.84 | 0.90 | 0.87 | 0.62 | 0.41 | 0.21 | 0.16 | 640 | 209 |
| A57208Hi | 1.04 | 0.87 | 0.71 | 0.48 | 0.27 | 0.13 | 0.10 | 284 | 210 |
| A57220Hi | n.d. | 1.02 | 0.62 | 0.60 | 0.45 | 0.24 | 0.14 | 514 | 222 |
| A57222Hi | 1.06 | 0.68 | 0.81 | 0.59 | 0.45 | 0.26 | 0.24 | 429 | 224 |
| A57239Hi | 0.99 | 0.99 | 0.83 | 0.80 | 0.49 | 0.24 | 0.16 | 920 | 241 |
| A57248Hi | 1.11 | 0.77 | 0.74 | 0.48 | 0.55 | 0.38 | 0.40 | 113 | 250 |
| A57147Hi | n.d. | 0.68 | 0.76 | 0.50 | 0.36 | 0.23 | 0.26 | 238 | 149 |
| A57252H | 0.81 | 0.77 | 0.71 | 0.59 | 0.38 | 0.23 | 0.13 | 1852 | 254 |
| A57253H | 0.76 | 0.79 | 0.83 | 0.62 | 0.47 | 0.28 | 0.18 | 347 | 255 |
| A57254H | 0.81 | 1.03 | 0.68 | 0.89 | 0.55 | 0.33 | 0.19 | 6144 | 256 |
| A57237Hi | n.d. | 0.89 | 0.85 | 0.72 | 0.54 | 0.30 | 0.18 | 1462 | 239 |

### Example 3: Knockdown of Human LRRK2 mRNA in Transgenic Mice

The bioactivity of human LRRK2-targeting ASOs was tested in mice expressing human LRRK2 (The Jackson Laboratory: C57BL/6J-Tg(LRRK2*G2019S)2AMjff/J, Stain #: 018785). These mice express endogenous mouse LRRK2 as well as human LRRK2 which is under the control of the human promoter/enhancer regions within the bacterial artificial chromosome (BAC) transgene. The human LRRK2 expression can be detected in the central nervous system and periphery (Bieri et al., 2019; Sitzia et al., 2022).

Human LRRK2 transgenic mice were either treated with a single intracerebroventricular (ICV, n=11 mice) bolus dose of 100 g of LRRK2 antisense oligonucleotide or received PBS as the vehicle control group (n=4 mice). Fourteen days after ICV injections, mice were sacrificed, tissues were harvested, and total RNA was extracted and processed for detection of LRRK2 mRNA using real-time PCR analysis. Treatment with selected ASOs resulted in statistically significant reductions of human LRRK2 mRNA ranging from ˜40% to ˜90% in the CNS regions, compared to vehicle control.

Table 7 shows the treatment with an ASO (SEQ ID NO. 209) resulted in statistically significant reduction (˜45-50%) of human LRRK2 mRNA in the striatum and cortex areas of the central nervous system, compared to vehicle control and normalized to the expression of HPRT1, a house keeping gene (Table 7). In the study cohort no statistically significant changes of human LRRK2 mRNA levels were detected in peripheral tissues lung, liver, blood, and kidney.

Table 7 shows human LRRK2 mRNA knockdown in transgenic mice treated with SEQ ID NO. 209 ASO:

| | LRKK2 mRNA | | |
|---|---|---|---|
| Tissue | reduction [%] | SEM | p value |
| Striatum | 50.19 | 0.06 | 0.0010 |
| Cortex | 44.40 | 0.05 | 0.0101 |
| Lung | no reduction | 0.34 | 0.1288 |
| Kidney | no reduction | 0.22 | 0.9973 |
| Liver | no reduction | 0.19 | 0.5134 |
| Blood | no reduction | 0.15 | 0.8556 |

Table 7 shows reduction of human LRRK2 mRNA levels in different regions of the central nervous system and in peripheral tissues detected by real-time PCR 14 days after ICV injection of either 100 g of LRRK2 ASO1 (n=12-13) or PBS vehicle (n=4).

Gene expression was normalized to HPRT1, a house keeping gene, and compared to human LRRK2 mRNA levels detected in PBS vehicle-treated animals (n=4). Variance is displayed as standard error of the mean (SEM). One way ANOVA was used to determine statistical significance.

### EMBODIMENTS OF THE INVENTION

1. An oligonucleotide comprising 10 to 25 nucleotides, wherein the sequence of nucleotides of the oligonucleotide is at least 80 to 90% complementary to a corresponding region of a LRRK2 nucleic acid sequence and wherein at least one nucleotide is modified and the modification selected from a modified sugar and optionally a modified internucleoside linkage.
2. The oligonucleotide according to embodiment 1, wherein the LRRK2 nucleic acid is mRNA of leucine-rich repeat kinase 2 (LRRK2) of SEQ ID NO. 1 and/or pre-mRNA of LRRK2 of SEQ ID NO. 2 and/or a fragment thereof.
3. The oligonucleotide according to embodiment 1 or 2, wherein the modification is selected from the group consisting of LNA, ENA, cET, a 2′Fluoro modified nucleotide, a 2′O-Methyl modified nucleotide, a 2′O-Methoxy modified nucleotide, a FANA and a combination thereof.
4. The oligonucleotide according to any one of embodiments 1 to 3, wherein the modification is located in a sequence of at least 3 nucleotides or at least 5 nucleotides of the 5′-end and/or 3′-end of the oligonucleotide.
5. The oligonucleotide according to any one of embodiments 1 to 4, wherein the modification is located in a sequence of 5 nucleotides of the 5′-end and/or 3′-end of the oligonucleotide.
6. The oligonucleotide according to any one of embodiments 1 to 5, wherein the internucleoside linkage is selected from the group consisting of phosphodiester, phosphorothioate, methylphosphonate, methyl phosphorothioate, phosphoramidate, phosphorodiamidate, thiophosphoramidate, mesyl phosphoramidate or a combination thereof
7. An oligonucleotide comprising 10 to 25 nucleotides, at least one nucleotide having a bridged nucleic acid modification selected from the group consisting of LNA, ENA, cET, a 2′Fluoro modified nucleotide, a 2′O-Methyl modified nucleotide, a 2′O-Methoxy modified nucleotide, a FANA and a combination thereof, wherein said modification is located in a sequence of 5 nucleotides of the 5′-end and/or 3′-end of the oligonucleotide and wherein the oligonucleotide hybridizes with a transcript or an mRNA of leucine-rich repeat kinase 2 (LRRK2) of SEQ ID NO. 1 and/or with pre-mRNA of LRRK2 of SEQ ID NO. 2 and/or a fragment thereof.
8. The oligonucleotide according to any one of embodiments 1 to 7, wherein the oligonucleotide hybridizes outside a hybridizing active region or within a hybridizing active region of position 27001 to 27500, position 102001 to 102500, position 80501 to 81000, position 24501 to 25000, position 114001 to 114500, position 92001 to 92500, position 99501 to 100000, position 67001 to 67500, position 15001 to 15500, position 24001 to 24500, position 26501 to 27000, position 79501 to 80000, position 87001 to 87500, position 120501 to 121000, position 142001 to 142500, position 135001 to 135500, position 71001 to 71500, position 140501 to 141000 or a combination thereof.
9. The oligonucleotide according to and one of embodiments 1 to 8, wherein the oligonucleotide comprises SEQ ID NO. 69, SEQ ID NO. 210, SEQ ID NO. 164, SEQ ID NO. 254, SEQ ID NO. 222, SEQ ID NO. 186, SEQ ID NO. 204, SEQ ID NO. 142, SEQ ID NO. 209, SEQ ID NO. 255, SEQ ID NO. 45, SEQ ID NO. 46, SEQ ID NO. 60, SEQ ID NO. 66, SEQ ID NO. 162, SEQ ID NO. 171, SEQ ID NO. 224, SEQ ID NO. 241, SEQ ID NO. 250, SEQ ID NO. 149, SEQ ID NO. 256, SEQ ID NO. 239, or a combination thereof.
10. The oligonucleotide according to any one of embodiments 1 to 9, wherein the oligonucleotide is selected from the group consisting of

```
  (A57067Hi; SEQ ID NO. 69)
+A*+A*+T*A*C*T*A*C*T*A*T*T*T*G*T*T*+T*+C*+C,
  (A57208Hi; SEQ ID NO. 210)
+T*+A*+C*A*A*T*A*T*T*T*A*T*G*G*T*T*+C*+T*+C,
  (A57162Hi; SEQ ID NO. 164)
+G*+T*+C*A*T*A*A*G*T*T*G*T*C*A*G*+T*+A*+T,
  (A57252H; SEQ ID NO. 254)
+G*+A*+C*T*T*C*G*T*T*T*A*A*T*A*C*+C*+A*+G,
  (A57220Hi; SEQ ID NO. 222)
+C*+T*+T*T*G*C*T*T*A*T*T*C*G*T*G*+A*+C*+T,
  (A57184Hi; SEQ ID NO. 186)
+T*+T*+T*A*T*T*G*C*A*G*T*C*A*T*A*G*+T*+G*+A,
  (A57202Hi; SEQ ID NO. 204)
+C*+T*+T*T*G*T*A*A*A*T*G*A*C*G*T*T*+T*+C*+C,
  (A57140Hi, SEQ ID NO. 142)
+T*+G*+A*A*A*T*G*T*C*T*A*T*T*A*G*C*+A*+C*+A,
  (A57207Hi; SEQ ID NO. 209)
+A*+C*+A*A*T*A*T*T*T*A*T*G*G*T*T*C*+T*+C*+A,
  (A57253H; SEQ ID NO. 255)
+G*+G*+A*C*T*T*C*G*T*T*T*A*A*T*A*C*+C*+A*+G,
  (A57043Hi; SEQ ID NO. 45)
+C*+G*+G*C*T*A*A*A*T*G*T*T*T*C*A*G*+T*+T*+C,
  (A57044Hi; SEQ ID NO. 46)
+T*+C*+G*G*C*T*A*A*A*T*G*T*T*T*C*+A*+G*+T,
  (A57058Hi; SEQ ID NO. 60)
+G*+T*+G*T*A*A*C*T*A*A*G*T*G*A*T*+C*+T*+C,
  (A57064Hi; SEQ ID NO. 66)
+G*+A*+T*A*T*T*G*A*C*A*A*C*C*T*G*+C*+T*+G,
  (A57160Hi; SEQ ID NO. 162)
+T*+G*+T*G*T*T*A*C*C*A*T*A*T*C*C*A*+T*+T*+T,
  (A57169Hi; SEQ ID NO. 171)
+C*+T*+G*T*G*A*C*T*T*T*G*C*A*T*C*A*+A*+C*+T,
  (A57222Hi; SEQ ID NO. 224)
+C*+A*+A*T*G*T*A*T*A*C*C*A*T*G*C*+A*+G*+T,
  (A57239Hi; SEQ ID NO. 241)
+T*+T*+C*C*T*G*A*T*T*A*T*A*C*C*A*T*+A*+T*+G,
  (A57248Hi; SEQ ID NO. 250)
+A*+T*+G*C*T*C*T*T*A*T*C*C*A*C*A*A*+T*+C*+C,
  (A57147Hi; SEQ ID NO. 149)
+T*+T*+T*G*A*T*C*T*G*T*A*G*T*A*C*A*+T*+A*+C,
  (A57254H; SEQ ID NO. 256)
+A*+T*+G*G*A*C*T*T*C*G*T*T*T*A*A*T*+A*+C*+C,
  (A57237Hi; SEQ ID NO. 239)
+G*+G*+A*C*A*T*G*T*A*T*C*T*T*A*A*T*+A*+G*+T,
```

and a combination thereof, wherein + indicates an LNA nucleotide and * indicates a phosphorothioate (PTO) linkage between the nucleotides.
11. The oligonucleotide according to any one of embodiments 1 to 10, wherein the oligonucleotide has 80 to 99%, 85 to 98%, 90 to 95 or 93% sequence identity to any one of the oligonucleotides of SEQ ID NO. 3-SEQ ID NO. 5301.
12. The oligonucleotide according to any one of embodiments 1 to 11, wherein said oligonucleotide comprises at least 7, at least 8, at least 9, at least 10, at least 11, at least 12, at least 13, at least 14, at least 15, at least 16, at least 17, at least 18, at least 19, at least 20, at least 21 or at least 22 contiguous nucleotides of any one of the oligonucleotides of SEQ ID NO. 3-SEQ ID NO. 5301.
13. The oligonucleotide according to any one of embodiments 1 to 12, wherein the oligonucleotide inhibits the expression of LRRK2, a LRRK2 mRNA, a LRRK2 pre-mRNA or a combination thereof at a nanomolar or micromolar concentration.
14. A pharmaceutical composition comprising an oligonucleotide according to any one of embodiments 1 to 13 and a pharmaceutically acceptable carrier, excipient, adjuvant, or a combination thereof.
15. The pharmaceutical composition according to embodiment 14, further comprising a therapeutically active agent.
16. The oligonucleotide according to any one of embodiments 1 to 13, or the pharmaceutical composition according to embodiment 14 or 15 for use in a method of preventing and/or treating a human disease such as a neurodegenerative disease.
17. The oligonucleotide or the pharmaceutical composition according to embodiment 16, wherein the pharmaceutically acceptable carrier, excipient, adjuvant, or a combination thereof, or the therapeutically active agent is administrated separately before or after delivery of the oligonucleotide, or is administered simultaneously via different delivery methods.
18. The oligonucleotide according to embodiment 16 or 17, wherein the disease is selected from the group consisting of Parkinson's disease (PD), Alzheimer's disease, Huntington's disease, amyotrophic lateral sclerosis, multiple sclerosis, multiple system atrophy, dementia with Lewy bodies (DLB), pure autonomic failure (PAF), frontotemporal dementia and prion disease.
19. The oligonucleotide or pharmaceutical composition for use according to any one of embodiments 16 to 18, wherein the oligonucleotide and/or the pharmaceutical composition is for local or systemic administration.
20. A method of preventing and/or treating a human disease comprising administering a therapeutically effective amount of an oligonucleotide according to any one of claims 1 to 13 or a pharmaceutical composition according to any one of embodiments 14 or 15.
21. The method of embodiment 20, wherein said human disease is selected from the group consisting of Parkinson's disease (PD), Alzheimer's disease, Huntington's disease, amyotrophic lateral sclerosis, multiple sclerosis, multiple system atrophy, dementia with Lewy bodies (DLB), pure autonomic failure (PAF), frontotemporal dementia and prion disease.
22. The method of embodiment 20 or 21, wherein said oligonucleotide or pharmaceutical composition is administered locally or systemically.

1. An oligonucleotide comprising 10 to 25 nucleotides, wherein the sequence of nucleotides of the oligonucleotide is at least 80 to 90% complementary to a corresponding region of a LRRK2 nucleic acid sequence and wherein at least one nucleotide is modified and the modification selected from a modified sugar and optionally a modified internucleoside linkage.

2. The oligonucleotide according to claim 1, wherein the LRRK2 nucleic acid is mRNA of leucine-rich repeat kinase 2 (LRRK2) of SEQ ID NO. 1 and/or pre-mRNA of LRRK2 of SEQ ID NO. 2 and/or a fragment thereof.

3. The oligonucleotide according to claim 1, wherein the modification is selected from the group consisting of LNA, ENA, cET, a 2′Fluoro modified nucleotide, a 2′O-Methyl modified nucleotide, a 2′O-Methoxy modified nucleotide, a FANA and a combination thereof.

4. The oligonucleotide according to claim 1, wherein the modification is located in a sequence of at least 3 nucleotides or at least 5 nucleotides of the 5′-end and/or 3′-end of the oligonucleotide.

5. The oligonucleotide according to claim 1, wherein the modification is located in a sequence of 5 nucleotides of the 5′-end and/or 3′-end of the oligonucleotide.

6. The oligonucleotide comprising 10 to 25 nucleotides according to claim 1, at least one nucleotide having a bridged nucleic acid modification selected from the group consisting of LNA, ENA, cET, a 2′Fluoro modified nucleotide, a 2′O-Methyl modified nucleotide, a 2′O-Methoxy modified nucleotide, a FANA and a combination thereof, wherein said modification is located in a sequence of 5 nucleotides of the 5′-end and/or 3′-end of the oligonucleotide and wherein the oligonucleotide hybridizes with a transcript or an mRNA of leucine-rich repeat kinase 2 (LRRK2) of SEQ ID NO. 1 and/or with pre-mRNA of LRRK2 of SEQ ID NO. 2 and/or a fragment thereof.

7. The oligonucleotide according to claim 1, wherein the oligonucleotide hybridizes outside a hybridizing active region or within a hybridizing active region of position 27001 to 27500, position 102001 to 102500, position 80501 to 81000, position 24501 to 25000, position 114001 to 114500, position 92001 to 92500, position 99501 to 100000, position 67001 to 67500, position 15001 to 15500, position 24001 to 24500, position 26501 to 27000, position 79501 to 80000, position 87001 to 87500, position 120501 to 121000, position 142001 to 142500, position 135001 to 135500, position 71001 to 71500, position 140501 to 141000 or a combination thereof.

8. The oligonucleotide according to claim 1, wherein the oligonucleotide comprises SEQ ID NO. 69, SEQ ID NO. 210, SEQ ID NO. 164, SEQ ID NO. 254, SEQ ID NO. 222, SEQ ID NO. 186, SEQ ID NO. 204, SEQ ID NO. 142, SEQ ID NO. 209, SEQ ID NO. 255, SEQ ID NO. 45, SEQ ID NO. 46, SEQ ID NO. 60, SEQ ID NO. 66, SEQ ID NO. 162, SEQ ID NO. 171, SEQ ID NO. 224, SEQ ID NO. 241, SEQ ID NO. 250, SEQ ID NO. 149, SEQ ID NO. 256, SEQ ID NO. 239, or a combination thereof.

9. The oligonucleotide according to claim 1, wherein the oligonucleotide is selected from the group consisting of
```
  (A57067Hi; SEQ ID NO. 69)
+A*+A*+T*A*C*T*A*C*T*A*T*T*T*G*T*T*+T*+C*+C,
  (A57208Hi; SEQ ID NO. 210)
+T*+A*+C*A*A*T*A*T*T*T*A*T*G*G*T*T*+C*+T*+C,
  (A57162Hi; SEQ ID NO. 164)
+G*+T*+C*A*T*A*A*G*T*T*G*T*C*A*G*+T*+A*+T,
  (A57252H; SEQ ID NO. 254)
+G*+A*+C*T*T*C*G*T*T*T*A*A*T*A*C*+C*+A*+G,
  (A57220Hi; SEQ ID NO. 222)
+C*+T*+T*T*G*C*T*T*A*T*T*C*G*T*G*+A*+C*+T,
  (A57184Hi; SEQ ID NO. 186)
+T*+T*+T*A*T*T*G*C*A*G*T*C*A*T*A*G*+T*+G*+A,
  (A57202Hi; SEQ ID NO. 204)
+C*+T*+T*T*G*T*A*A*A*T*G*A*C*G*T*T*+T*+C*+C,
  (A57140Hi, SEQ ID NO. 142)
+T*+G*+A*A*A*T*G*T*C*T*A*T*T*A*G*C*+A*+C*+A,
  (A57207Hi; SEQ ID NO. 209)
+A*+C*+A*A*T*A*T*T*T*A*T*G*G*T*T*C*+T*+C*+A,
  (A57253H; SEQ ID NO. 255)
+G*+G*+A*C*T*T*C*G*T*T*T*A*A*T*A*C*+C*+A*+G,
  (A57043Hi; SEQ ID NO. 45)
+C*+G*+G*C*T*A*A*A*T*G*T*T*T*C*A*G*+T*+T*+C,
  (A57044Hi; SEQ ID NO. 46)
+T*+C*+G*G*C*T*A*A*A*T*G*T*T*T*C*+A*+G*+T,
  (A57058Hi; SEQ ID NO. 60)
+G*+T*+G*T*A*A*C*T*A*A*G*T*G*A*T*+C*+T*+C,
  (A57064Hi; SEQ ID NO. 66)
+G*+A*+T*A*T*T*G*A*C*A*A*C*C*T*G*+C*+T*+G,
  (A57160Hi; SEQ ID NO. 162)
+T*+G*+T*G*T*T*A*C*C*A*T*A*T*C*C*A*+T*+T*+T,
  (A57169Hi; SEQ ID NO. 171)
+C*+T*+G*T*G*A*C*T*T*T*G*C*A*T*C*A*+A*+C*+T,
  (A57222Hi; SEQ ID NO. 224)
+C*+A*+A*T*G*T*A*T*A*C*C*A*T*G*C*+A*+G*+T,
  (A57239Hi; SEQ ID NO. 241)
+T*+T*+C*C*T*G*A*T*T*A*T*A*C*C*A*T*+A*+T*+G,
  (A57248Hi; SEQ ID NO. 250)
+A*+T*+G*C*T*C*T*T*A*T*C*C*A*C*A*A*+T*+C*+C,
  (A57147Hi; SEQ ID NO. 149)
+T*+T*+T*G*A*T*C*T*G*T*A*G*T*A*C*A*+T*+A*+C,
  (A57254H; SEQ ID NO. 256)
+A*+T*+G*G*A*C*T*T*C*G*T*T*T*A*A*T*+A*+C*+C,
  (A57237Hi; SEQ ID NO. 239)
+G*+G*+A*C*A*T*G*T*A*T*C*T*T*A*A*T*+A*+G*+T,
```
and a combination thereof, wherein + indicates an LNA nucleotide and * indicates a phosphorothioate (PTO) linkage between the nucleotides.

10. The oligonucleotide according to claim 1, wherein the oligonucleotide has 80 to 99%, 85 to 98%, 90 to 95 or 93% sequence identity to any one of the oligonucleotides of SEQ ID NO. 3-SEQ ID NO. 5301.

11. The oligonucleotide according to claim 1, wherein said oligonucleotide comprises at least 7, at least 8, at least 9, at least 10, at least 11, at least 12, at least 13, at least 14, at least 15, at least 16, at least 17, at least 18, at least 19, at least 20, at least 21 or at least 22 contiguous nucleotides of any one of the oligonucleotides of SEQ ID NO. 3-SEQ ID NO. 5301.

12. The oligonucleotide according to claim 1, wherein the oligonucleotide inhibits the expression of LRRK2, a LRRK2 mRNA, a LRRK2 pre-mRNA or a combination thereof at a nanomolar or micromolar concentration.

13. A pharmaceutical composition comprising an oligonucleotide according to claim 1 and a pharmaceutically acceptable carrier, excipient, adjuvant, or a combination thereof.

14. The pharmaceutical composition according to claim 13, further comprising a therapeutically active agent.

15. The method according to claim 19 wherein the human disease is a neurodegenerative disease.

16. The pharmaceutical composition according to claim 14, wherein the pharmaceutically acceptable carrier, excipient, adjuvant, or a combination thereof, or the therapeutically active agent is administered separately before or after delivery of the oligonucleotide, or is administered simultaneously via different delivery methods.

17. The method according to claim 15, wherein the neurodegenerative disease is selected from the group consisting of Parkinson's disease (PD), Alzheimer's disease, Huntington's disease, amyotrophic lateral sclerosis, multiple sclerosis, multiple system atrophy, dementia with Lewy bodies (DLB), pure autonomic failure (PAF), frontotemporal dementia and prion disease.

18. The pharmaceutical composition according to claim 13, wherein the pharmaceutical composition is for local or systemic administration.

19. A method of preventing and/or treating a human disease comprising administering to a human subject in need thereof a therapeutically effective amount of an oligonucleotide according to claim 1.

20. (canceled)

21. The method of claim 19, wherein said oligonucleotide is administered locally or systemically.
